SEQUENCE LISTING (620 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 241 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERT

SEQ ID NO: 3 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHHHHHH

SEQ ID NO: 4 - protein, 475 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 249 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTDYKDDDDK

SEQ ID NO: 6 - protein, 405 aa, Homo sapiens
MGWSCIILFLVATATGVHSLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQKLENLYFQGIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 7 - protein, 8 aa, Homo sapiens
GFTFDDYA

SEQ ID NO: 8 - protein, 8 aa, Homo sapiens
ISWKSNII

SEQ ID NO: 9 - protein, 15 aa, Homo sapiens
ARDITGSGSYGWFDP

SEQ ID NO: 10 - protein, 5 aa, Homo sapiens
DYAMH

SEQ ID NO: 11 - protein, 17 aa, Homo sapiens
GISWKSNIIGYADSVKG

SEQ ID NO: 12 - protein, 13 aa, Homo sapiens
DITGSGSYGWFDP

SEQ ID NO: 13 - protein, 122 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSS

SEQ ID NO: 14 - DNA, 508 bp, Homo sapiens
  source 1..508: mol_type = unassigned DNA
caagaaaaagcttgccgccaccatggagtttgggctgagctggattttccttttggctattttaaaaggtgtccagtgtgaagtacaattggtggagtccgggggaggcttggtacagcctggcaggtccctgagactctcctgtgcagcctctggattcacctttgatgattatgccatgcactgggtccgacaaactccagggaagggcctggagtgggtctcaggtataagttggaagagtaatatcataggctatgcggactctgtgaagggccgattcaccatctccagagacaacgccaagaactccctgtatctgcaaatgaacagtctgagagctgaggacacggccttgtattattgtgcaagagatataacgggttcggggagttatggctggttcgacccctggggccagggaaccctggtcaccgtctcctcagccaaaacgacacccccatctgtctatccactggcccctgaatctgctaaaactcagcctccg

SEQ ID NO: 15 - protein, 449 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 1356 bp, Homo sapiens
  source 1..1356: mol_type = unassigned DNA
gaagtgcagctggtggaatctggcggcggactggtgcagcctggcagatccctgagactgtcttgtgccgcctccggcttcaccttcgacgactacgctatgcactgggtgcgacagacccctggcaagggcctggaatgggtgtccggcatctcctggaagtccaacatcatcggctacgccgactccgtgaagggccggttcaccatctcccgggacaacgccaagaactccctgtacctgcagatgaacagcctgcgggccgaggacaccgccctgtactactgcgccagagacatcaccggctccggctcctacggatggttcgatccttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 17 - protein, 6 aa, Homo sapiens
QSISSY

SEQ ID NO: 18 - skipped

SEQ ID NO: 19 - protein, 9 aa, Homo sapiens
QQSYSNPIT

SEQ ID NO: 20 - protein, 11 aa, Homo sapiens
RASQSISSYLN

SEQ ID NO: 21 - protein, 7 aa, Homo sapiens
VASSLQS

SEQ ID NO: 22 - protein, 9 aa, Homo sapiens
QQSYSNPIT

SEQ ID NO: 23 - protein, 107 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIK

SEQ ID NO: 24 - DNA, 321 bp, Homo sapiens
  source 1..321: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaa

SEQ ID NO: 25 - protein, 214 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, Homo sapiens
  source 1..642: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 27 - protein, 8 aa, Homo sapiens
GFTFDDYA

SEQ ID NO: 28 - protein, 8 aa, Homo sapiens
ISWIRTGI

SEQ ID NO: 29 - protein, 16 aa, Homo sapiens
AKDMKGSGTYGGWFDT

SEQ ID NO: 30 - protein, 5 aa, Homo sapiens
DYAMH

SEQ ID NO: 31 - protein, 17 aa, Homo sapiens
GISWIRTGIGYADSVKG

SEQ ID NO: 32 - protein, 14 aa, Homo sapiens
DMKGSGTYGGWFDT

SEQ ID NO: 33 - protein, 123 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSS

SEQ ID NO: 34 - DNA, 482 bp, Homo sapiens
  source 1..482: mol_type = unassigned DNA
aagcttgccgccaccatggagtttgggctgagctggattttccttttggctattttaaaaggtgtccagtgtgaagtgcagctggtggagtctgggggaggcttggtgcagcctggcaggtccctgagactctcctgtgcagcctctggattcacctttgatgattatgccatgcactgggtccggcaagttccagggaagggcctggaatgggtctcaggcattagttggattcgtactggcataggctatgcggactctgtgaagggccgattcaccattttcagagacaacgccaagaattccctgtatctgcaaatgaacagtctgagagctgaggacacggccttgtattactgtgcaaaagatatgaagggttcggggacttatggggggtggttcgacacctggggccagggaaccctggtcaccgtctcctcagccaaaacaacagccccatcggtctatccactggcccctgc

SEQ ID NO: 35 - protein, 450 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - DNA, 1359 bp, Homo sapiens
  source 1..1359: mol_type = unassigned DNA
gaagtgcagctggtggaatctggcggcggactggtgcagcctggcagatccctgagactgtcttgtgccgcctccggcttcaccttcgacgactacgctatgcactgggtgcgacaggtgccaggcaagggcctggaatgggtgtccggcatctcttggatccggaccggcatcggctacgccgactctgtgaagggccggttcaccatcttccgggacaacgccaagaactccctgtacctgcagatgaacagcctgcgggccgaggacaccgccctgtactactgcgccaaggacatgaagggctccggcacctacggcggatggttcgatacttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 37 - protein, 6 aa, Homo sapiens
QSISSY

SEQ ID NO: 38 - skipped

SEQ ID NO: 39 - protein, 9 aa, Homo sapiens
QQSYSTPIT

SEQ ID NO: 40 - protein, 11 aa, Homo sapiens
RASQSISSYLN

SEQ ID NO: 41 - protein, 7 aa, Homo sapiens
VASSLQS

SEQ ID NO: 42 - protein, 9 aa, Homo sapiens
QQSYSTPIT

SEQ ID NO: 43 - protein, 107 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIK

SEQ ID NO: 44 - DNA, 418 bp, Homo sapiens
  source 1..418: mol_type = unassigned DNA
aaagcttgccgccaccatgaggctccctgctcagcttctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaaactcctgatctatgttgcatccagtttgcaaagtggggtcccatcaaggttcagtggcagtggatctgggacagatttcactctcactatcagcagtctgcaacctgaagattttgcaacttactactgtcaacagagttacagtaccccgatcaccttcggccaagggacacgtctggagatcaaacgtacggatgctgcaccaact

SEQ ID NO: 45 - protein, 214 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 642 bp, Homo sapiens
  source 1..642: mol_type = unassigned DNA
gacatccagatgacccagtccccctccagcctgtctgcttccgtgggcgacagagtgaccatcacctgtcgggcctcccagtccatctcctcctacctgaactggtatcagcagaagcccggcaaggcccccaagctgctgatctacgtggccagctctctgcagtccggcgtgccctctagattctccggctctggctctggcaccgactttaccctgaccatcagctccctgcagcccgaggacttcgccacctactactgccagcagtcctactccacccctatcaccttcggccagggcacccggctggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 47 - protein, 450 aa, Homo sapiens
EVQLVESGGGLVQPGRSLRLSCAASGFTFDDYAMHWVRQAPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 450 aa, Homo sapiens
EVQLVESGGGLVQPGRSLRLSCAASGFTFDDYAMHWVRQVPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 448 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Homo sapiens
DIQMTQSPSSLSASVGDRVTITCRASQSISSYLNWYQQKPGKAPKLFIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 8 aa, Homo sapiens
GFTFSSYW

SEQ ID NO: 53 - protein, 8 aa, Homo sapiens
IKEDGSEK

SEQ ID NO: 54 - protein, 12 aa, Homo sapiens
ARNRLYSDFLDN

SEQ ID NO: 55 - protein, 5 aa, Homo sapiens
SYWMS

SEQ ID NO: 56 - protein, 17 aa, Homo sapiens
NIKEDGSEKYYVDSVKG

SEQ ID NO: 57 - protein, 10 aa, Homo sapiens
NRLYSDFLDN

SEQ ID NO: 58 - protein, 119 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSS

SEQ ID NO: 59 - DNA, 358 bp, Homo sapiens
  source 1..358: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcag

SEQ ID NO: 60 - protein, 449 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1347 bp, Homo sapiens
  source 1..1347: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 62 - protein, 6 aa, Homo sapiens
QGVSSW

SEQ ID NO: 63 - skipped

SEQ ID NO: 64 - protein, 9 aa, Homo sapiens
QQANSIPFT

SEQ ID NO: 65 - protein, 11 aa, Homo sapiens
RASQGVSSWLA

SEQ ID NO: 66 - protein, 7 aa, Homo sapiens
GASSLQS

SEQ ID NO: 67 - protein, 9 aa, Homo sapiens
QQANSIPFT

SEQ ID NO: 68 - protein, 107 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIK

SEQ ID NO: 69 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaac

SEQ ID NO: 70 - protein, 214 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 72 - protein, 8 aa, Homo sapiens
GFTFSSYW

SEQ ID NO: 73 - protein, 8 aa, Homo sapiens
IKEDGSEK

SEQ ID NO: 74 - protein, 12 aa, Homo sapiens
ARVRLYSDFLDY

SEQ ID NO: 75 - protein, 5 aa, Homo sapiens
SYWMS

SEQ ID NO: 76 - protein, 17 aa, Homo sapiens
NIKEDGSEKYYVDSLKG

SEQ ID NO: 77 - protein, 10 aa, Homo sapiens
VRLYSDFLDY

SEQ ID NO: 78 - protein, 119 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSS

SEQ ID NO: 79 - DNA, 358 bp, Homo sapiens
  source 1..358: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcag

SEQ ID NO: 80 - protein, 449 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1347 bp, Homo sapiens
  source 1..1347: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 82 - protein, 6 aa, Homo sapiens
QGVSSW

SEQ ID NO: 83 - skipped

SEQ ID NO: 84 - protein, 9 aa, Homo sapiens
QQANSIPFT

SEQ ID NO: 85 - protein, 11 aa, Homo sapiens
RASQGVSSWLA

SEQ ID NO: 86 - protein, 7 aa, Homo sapiens
GASSLQS

SEQ ID NO: 87 - protein, 9 aa, Homo sapiens
QQANSIPFT

SEQ ID NO: 88 - protein, 107 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIK

SEQ ID NO: 89 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaac

SEQ ID NO: 90 - protein, 214 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 92 - protein, 9 aa, Homo sapiens
GGSIISSDW

SEQ ID NO: 93 - protein, 7 aa, Homo sapiens
IFHSGRT

SEQ ID NO: 94 - protein, 8 aa, Homo sapiens
ARDGSGSY

SEQ ID NO: 95 - protein, 6 aa, Homo sapiens
SSDWWN

SEQ ID NO: 96 - protein, 16 aa, Homo sapiens
EIFHSGRTNYNPSLKS

SEQ ID NO: 97 - protein, 6 aa, Homo sapiens
DGSGSY

SEQ ID NO: 98 - protein, 115 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSS

SEQ ID NO: 99 - DNA, 346 bp, Homo sapiens
  source 1..346: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcag

SEQ ID NO: 100 - protein, 445 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1335 bp, Homo sapiens
  source 1..1335: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 102 - protein, 12 aa, Homo sapiens
QSVLYSSNNKNY

SEQ ID NO: 103 - skipped

SEQ ID NO: 104 - protein, 8 aa, Homo sapiens
QQYYSNRS

SEQ ID NO: 105 - protein, 17 aa, Homo sapiens
KSSQSVLYSSNNKNYLA

SEQ ID NO: 106 - protein, 7 aa, Homo sapiens
WASTRES

SEQ ID NO: 107 - protein, 8 aa, Homo sapiens
QQYYSNRS

SEQ ID NO: 108 - protein, 112 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIK

SEQ ID NO: 109 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaac

SEQ ID NO: 110 - protein, 219 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 112 - protein, 8 aa, Homo sapiens
GFTFSSYW

SEQ ID NO: 113 - protein, 8 aa, Homo sapiens
IKEDGSEK

SEQ ID NO: 114 - protein, 12 aa, Homo sapiens
ARNRLYSDFLDN

SEQ ID NO: 115 - protein, 5 aa, Homo sapiens
SYWMS

SEQ ID NO: 116 - protein, 17 aa, Homo sapiens
NIKEDGSEKYYVDSVKG

SEQ ID NO: 117 - protein, 10 aa, Homo sapiens
NRLYSDFLDN

SEQ ID NO: 118 - protein, 119 aa, Homo sapiens; identical to SEQ ID NO: 58

SEQ ID NO: 119 - DNA, 358 bp, Homo sapiens; identical to SEQ ID NO: 59
  source 1..358: mol_type = unassigned DNA

SEQ ID NO: 120 - protein, 449 aa, Homo sapiens; identical to SEQ ID NO: 60

SEQ ID NO: 121 - DNA, 1347 bp, Homo sapiens; identical to SEQ ID NO: 61
  source 1..1347: mol_type = unassigned DNA

SEQ ID NO: 122 - protein, 6 aa, Homo sapiens
QGVSSW

SEQ ID NO: 123 - skipped

SEQ ID NO: 124 - protein, 9 aa, Homo sapiens
QQANSIPFT

SEQ ID NO: 125 - protein, 11 aa, Homo sapiens
RASQGVSSWLA

SEQ ID NO: 126 - protein, 7 aa, Homo sapiens
GASSLQS

SEQ ID NO: 127 - protein, 9 aa, Homo sapiens
QQANSIPFT

SEQ ID NO: 128 - protein, 107 aa, Homo sapiens; identical to SEQ ID NO: 68

SEQ ID NO: 129 - DNA, 322 bp, Homo sapiens; identical to SEQ ID NO: 69
  source 1..322: mol_type = unassigned DNA

SEQ ID NO: 130 - protein, 214 aa, Homo sapiens; identical to SEQ ID NO: 70

SEQ ID NO: 131 - DNA, 642 bp, Homo sapiens; identical to SEQ ID NO: 71
  source 1..642: mol_type = unassigned DNA

SEQ ID NO: 132 - protein, 8 aa, Homo sapiens
GFTFRIYG

SEQ ID NO: 133 - protein, 8 aa, Homo sapiens
IWYDGSNK

SEQ ID NO: 134 - protein, 11 aa, Homo sapiens
ARDMDYFGMDV

SEQ ID NO: 135 - protein, 5 aa, Homo sapiens
IYGMH

SEQ ID NO: 136 - protein, 17 aa, Homo sapiens
VIWYDGSNKYYADSVKG

SEQ ID NO: 137 - protein, 9 aa, Homo sapiens
DMDYFGMDV

SEQ ID NO: 138 - protein, 118 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSS

SEQ ID NO: 139 - DNA, 355 bp, Homo sapiens
  source 1..355: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcag

SEQ ID NO: 140 - protein, 448 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1344 bp, Homo sapiens
  source 1..1344: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 142 - protein, 6 aa, Homo sapiens
QGIRND

SEQ ID NO: 143 - skipped

SEQ ID NO: 144 - protein, 9 aa, Homo sapiens
LQHNSYPRT

SEQ ID NO: 145 - protein, 11 aa, Homo sapiens
RASQGIRNDLG

SEQ ID NO: 146 - protein, 7 aa, Homo sapiens
AASSLQS

SEQ ID NO: 147 - protein, 9 aa, Homo sapiens
LQHNSYPRT

SEQ ID NO: 148 - protein, 107 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 149 - DNA, 322 bp, Homo sapiens
  source 1..322: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaac

SEQ ID NO: 150 - protein, 214 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, Homo sapiens
  source 1..642: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 152 - protein, 9 aa, Homo sapiens
GGSISSSDW

SEQ ID NO: 153 - protein, 7 aa, Homo sapiens
IFHSGNT

SEQ ID NO: 154 - protein, 8 aa, Homo sapiens
VRDGSGSY

SEQ ID NO: 155 - protein, 6 aa, Homo sapiens
SSDWWS

SEQ ID NO: 156 - protein, 16 aa, Homo sapiens
EIFHSGNTNYNPSLKS

SEQ ID NO: 157 - protein, 6 aa, Homo sapiens
DGSGSY

SEQ ID NO: 158 - protein, 115 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSS

SEQ ID NO: 159 - DNA, 346 bp, Homo sapiens
  source 1..346: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcag

SEQ ID NO: 160 - protein, 445 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 1335 bp, Homo sapiens
  source 1..1335: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 162 - protein, 12 aa, Homo sapiens
QSVLYSSNNKNY

SEQ ID NO: 163 - skipped

SEQ ID NO: 164 - protein, 8 aa, Homo sapiens
QQYYSTRS

SEQ ID NO: 165 - protein, 17 aa, Homo sapiens
KSSQSVLYSSNNKNYLA

SEQ ID NO: 166 - protein, 7 aa, Homo sapiens
WASTRES

SEQ ID NO: 167 - protein, 8 aa, Homo sapiens
QQYYSTRS

SEQ ID NO: 168 - protein, 112 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIK

SEQ ID NO: 169 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaac

SEQ ID NO: 170 - protein, 219 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 172 - protein, 10 aa, Homo sapiens
GGSISSSSYY

SEQ ID NO: 173 - protein, 7 aa, Homo sapiens
IYSTGYT

SEQ ID NO: 174 - protein, 13 aa, Homo sapiens
AISTAAGPEYFHR

SEQ ID NO: 175 - protein, 7 aa, Homo sapiens
SSSYYCG

SEQ ID NO: 176 - protein, 16 aa, Homo sapiens
SIYSTGYTYYNPSLKS

SEQ ID NO: 177 - protein, 11 aa, Homo sapiens
STAAGPEYFHR

SEQ ID NO: 178 - protein, 120 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSS

SEQ ID NO: 179 - DNA, 361 bp, Homo sapiens
  source 1..361: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcag

SEQ ID NO: 180 - protein, 450 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1350 bp, Homo sapiens
  source 1..1350: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 182 - protein, 12 aa, Homo sapiens
QSVLYSSNSKNF

SEQ ID NO: 183 - skipped

SEQ ID NO: 184 - protein, 9 aa, Homo sapiens
QQYYSTPRT

SEQ ID NO: 185 - protein, 17 aa, Homo sapiens
KSSQSVLYSSNSKNFLA

SEQ ID NO: 186 - protein, 7 aa, Homo sapiens
WASTRGS

SEQ ID NO: 187 - protein, 9 aa, Homo sapiens
QQYYSTPRT

SEQ ID NO: 188 - protein, 113 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIK

SEQ ID NO: 189 - DNA, 340 bp, Homo sapiens
  source 1..340: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaac

SEQ ID NO: 190 - protein, 220 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 660 bp, Homo sapiens
  source 1..660: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 192 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 193 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 195 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 197 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 198 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 199 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - DNA, 981 bp, Homo sapiens
  source 1..981: mol_type = unassigned DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 201 - DNA, 981 bp, Homo sapiens
  source 1..981: mol_type = unassigned DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 202 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 203 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 205 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - DNA, 321 bp, Homo sapiens
  source 1..321: mol_type = unassigned DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 207 - protein, 107 aa, Homo sapiens [sequence omitted]

SEQ ID NO: 208 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 209 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 210 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 211 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 212 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 213 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 214 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 215 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 216 - DNA, 312 bp, Homo sapiens
  source 1..312: mol_type = unassigned DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 217 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 218 - DNA, 318 bp, Homo sapiens
  source 1..318: mol_type = unassigned DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 219 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 220 - DNA, 318 bp, Homo sapiens; identical to SEQ ID NO: 218
  source 1..318: mol_type = unassigned DNA

SEQ ID NO: 221 - DNA, 318 bp, Homo sapiens
  source 1..318: mol_type = unassigned DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 222 - DNA, 318 bp, Homo sapiens
  source 1..318: mol_type = unassigned DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 223 - protein, 106 aa, Homo sapiens; identical to SEQ ID NO: 219

SEQ ID NO: 224 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 225 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 226 - DNA, 312 bp, Homo sapiens
  source 1..312: mol_type = unassigned DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 227 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 228 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 229 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 230 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 231 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 232 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 233 - protein, 106 aa, Homo sapiens; identical to SEQ ID NO: 225

SEQ ID NO: 234 - DNA, 318 bp, Homo sapiens
  source 1..318: mol_type = unassigned DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 235 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 236 - DNA, 318 bp, Homo sapiens
  source 1..318: mol_type = unassigned DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 237 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 238 - protein, 8 aa, Homo sapiens
GFTFSDYY

SEQ ID NO: 239 - protein, 8 aa, Homo sapiens
ISTSGSTI

SEQ ID NO: 240 - protein, 16 aa, Homo sapiens
ARGITGTNFYHYGLGV

SEQ ID NO: 241 - protein, 5 aa, Homo sapiens
DYYMS

SEQ ID NO: 242 - protein, 17 aa, Homo sapiens
YISTSGSTIYYADSVKG

SEQ ID NO: 243 - protein, 14 aa, Homo sapiens
GITGTNFYHYGLGV

SEQ ID NO: 244 - protein, 123 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSS

SEQ ID NO: 245 - DNA, 370 bp, Homo sapiens
  source 1..370: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcag

SEQ ID NO: 246 - protein, 453 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1359 bp, Homo sapiens
  source 1..1359: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 248 - protein, 6 aa, Homo sapiens
QGINSW

SEQ ID NO: 249 - skipped

SEQ ID NO: 250 - protein, 9 aa, Homo sapiens
QQVNSFPLT

SEQ ID NO: 251 - protein, 11 aa, Homo sapiens
RASQGINSWLA

SEQ ID NO: 252 - protein, 7 aa, Homo sapiens
AASTLQS

SEQ ID NO: 253 - protein, 9 aa, Homo sapiens
QQVNSFPLT

SEQ ID NO: 254 - protein, 107 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIK

SEQ ID NO: 255 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaac

SEQ ID NO: 256 - protein, 214 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 258 - protein, 8 aa, Homo sapiens
GFTFSYYA

SEQ ID NO: 259 - protein, 8 aa, Homo sapiens
ISGGGGNT

SEQ ID NO: 260 - protein, 16 aa, Homo sapiens
AKDRMKQLVRAYYFDY

SEQ ID NO: 261 - protein, 5 aa, Homo sapiens
YYAMS

SEQ ID NO: 262 - protein, 17 aa, Homo sapiens
TISGGGGNTHYADSVKG

SEQ ID NO: 263 - protein, 14 aa, Homo sapiens
DRMKQLVRAYYFDY

SEQ ID NO: 264 - protein, 123 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSS

SEQ ID NO: 265 - DNA, 370 bp, Homo sapiens
  source 1..370: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcag

SEQ ID NO: 266 - protein, 453 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - DNA, 1359 bp, Homo sapiens
  source 1..1359: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 268 - protein, 6 aa, Homo sapiens
QDISTY

SEQ ID NO: 269 - skipped

SEQ ID NO: 270 - protein, 9 aa, Homo sapiens
QQLHTDPIT

SEQ ID NO: 271 - protein, 11 aa, Homo sapiens
WASQDISTYLG

SEQ ID NO: 272 - protein, 7 aa, Homo sapiens
GTSSLQS

SEQ ID NO: 273 - protein, 9 aa, Homo sapiens
QQLHTDPIT

SEQ ID NO: 274 - protein, 107 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIK

SEQ ID NO: 275 - DNA, 322 bp, Homo sapiens
  source 1..322: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaac

SEQ ID NO: 276 - protein, 214 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - DNA, 642 bp, Homo sapiens
  source 1..642: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 278 - protein, 8 aa, Homo sapiens
GFTFSSYW

SEQ ID NO: 279 - protein, 8 aa, Homo sapiens
IKQDGSEK

SEQ ID NO: 280 - protein, 12 aa, Homo sapiens
ARVRQWSDYSDY

SEQ ID NO: 281 - protein, 5 aa, Homo sapiens
SYWMN

SEQ ID NO: 282 - protein, 17 aa, Homo sapiens
NIKQDGSEKYYVDSVKG

SEQ ID NO: 283 - protein, 10 aa, Homo sapiens
VRQWSDYSDY

SEQ ID NO: 284 - protein, 119 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSS

SEQ ID NO: 285 - DNA, 358 bp, Homo sapiens
  source 1..358: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcag

SEQ ID NO: 286 - protein, 449 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - DNA, 1347 bp, Homo sapiens
  source 1..1347: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 288 - protein, 6 aa, Homo sapiens
QGISSW

SEQ ID NO: 289 - skipped

SEQ ID NO: 290 - protein, 9 aa, Homo sapiens
QQANSFPFT

SEQ ID NO: 291 - protein, 11 aa, Homo sapiens
RASQGISSWLA

SEQ ID NO: 292 - protein, 7 aa, Homo sapiens
AASSLQS

SEQ ID NO: 293 - protein, 9 aa, Homo sapiens
QQANSFPFT

SEQ ID NO: 294 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIK

SEQ ID NO: 295 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaac

SEQ ID NO: 296 - protein, 214 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 298 - protein, 214 aa, Homo sapiens
DIQMTQSPSSLSASVGDRVTITCRASQSISSYLNWYQQKPGKAPKLLIYYASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 453 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 347 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 301 - protein, 133 aa, Homo sapiens [sequence omitted]

SEQ ID NO: 302 - protein, 586 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 303 - protein, 14 aa, Homo sapiens
APTSTQLQLELLLD

SEQ ID NO: 304 - protein, 11 aa, Homo sapiens
TQLQLEHLLLD

SEQ ID NO: 305 - protein, 17 aa, Homo sapiens
APTSKKTQLQLEHLLLD

SEQ ID NO: 306 - protein, 19 aa, Homo sapiens
PTSSSTKKTQLQLEHLLLD

SEQ ID NO: 307 - protein, 18 aa, Homo sapiens
TSSSTKKTQLQLEHLLLD

SEQ ID NO: 308 - protein, 17 aa, Homo sapiens
SSSTKKTQLQLEHLLLD

SEQ ID NO: 309 - protein, 16 aa, Homo sapiens
SSTKKTQLQLEHLLLD

SEQ ID NO: 310 - protein, 15 aa, Homo sapiens
STKKTQLQLEHLLLD

SEQ ID NO: 311 - protein, 14 aa, Homo sapiens
TKKTQLQLEHLLLD

SEQ ID NO: 312 - protein, 13 aa, Homo sapiens
KKTQLQLEHLLLD

SEQ ID NO: 313 - protein, 12 aa, Homo sapiens
KTQLQLEHLLLD

SEQ ID NO: 314 - protein, 19 aa, Homo sapiens
APTSSSTKTQLQLEHLLLD

SEQ ID NO: 315 - protein, 18 aa, Homo sapiens
APTSSSTTQLQLEHLLLD

SEQ ID NO: 316 - protein, 17 aa, Homo sapiens
APTSSSTQLQLEHLLLD

SEQ ID NO: 317 - protein, 16 aa, Homo sapiens
APTSSTQLQLEHLLLD

SEQ ID NO: 318 - protein, 14 aa, Homo sapiens
APTTQLQLEHLLLD

SEQ ID NO: 319 - protein, 13 aa, Homo sapiens
APTQLQLEHLLLD

SEQ ID NO: 320 - protein, 12 aa, Homo sapiens
ATQLQLEHLLLD

SEQ ID NO: 321 - protein, 15 aa, Homo sapiens
ATKKTQLQLEHLLLD

SEQ ID NO: 322 - protein, 15 aa, Homo sapiens
APKKTQLQLEHLLLD

SEQ ID NO: 323 - protein, 15 aa, Homo sapiens
APTKTQLQLEHLLLD

SEQ ID NO: 324 - protein, 113 aa, Homo sapiens
LQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 325 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 326 - protein, 226 aa, Mus musculus
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHHHHHH

SEQ ID NO: 327 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 328 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 329 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 330 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 331 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 332 - protein, 133 aa, Homo sapiens [sequence omitted]

SEQ ID NO: 333 - protein, 127 aa, Homo sapiens [sequence omitted]

SEQ ID NO: 334 - protein, 171 aa, Homo sapiens
CDLPQNHGLLSRNTLVLLHQMRRISPFLCLKDRRDFRFPQEMVKGSQLQKAHVMSVLHEMLQQIFSLFHTERSSAAWNMTLLDQLHTELHQQLQHLETCLLQVVGEGESAGAISSPALTLRRYFQGIRVYLKEKKYSDCAWEVVRMEIMKSLFLSTNMQERLRSKDRDLGS

SEQ ID NO: 335 - protein, 177 aa, Homo sapiens
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 336 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 337 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 338 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 339 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 340 - protein, 330 aa, Homo sapiens [sequence omitted]

SEQ ID NO: 341 - DNA, 996 bp, Homo sapiens
  source 1..996: mol_type = unassigned DNA
gccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 342 - protein, 450 aa, Homo sapiens
EVQLVESGGGLVQPGRSLRLSCAASGFTFDDYAMHWVRQAPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 343 - protein, 8 aa, Homo sapiens
GFTFSNYA

SEQ ID NO: 344 - protein, 8 aa, Homo sapiens
ISFSGGTT

SEQ ID NO: 345 - protein, 14 aa, Homo sapiens
AKDEAPAGATFFDS

SEQ ID NO: 346 - protein, 5 aa, Homo sapiens
NYAMS

SEQ ID NO: 347 - protein, 17 aa, Homo sapiens
AISFSGGTTYYADSVKG

SEQ ID NO: 348 - protein, 12 aa, Homo sapiens
DEAPAGATFFDS

SEQ ID NO: 349 - protein, 121 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSS

SEQ ID NO: 350 - DNA, 364 bp, Homo sapiens
  source 1..364: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcag

SEQ ID NO: 351 - protein, 448 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 352 - DNA, 1344 bp, Homo sapiens
  source 1..1344: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 353 - protein, 6 aa, Homo sapiens
QGIRRW

SEQ ID NO: 354 - skipped

SEQ ID NO: 355 - protein, 9 aa, Homo sapiens
QQANSFPIT

SEQ ID NO: 356 - protein, 11 aa, Homo sapiens
RASQGIRRWLA

SEQ ID NO: 357 - protein, 7 aa, Homo sapiens
GASSLQS

SEQ ID NO: 358 - protein, 9 aa, Homo sapiens
QQANSFPIT

SEQ ID NO: 359 - protein, 107 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIK

SEQ ID NO: 360 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaac

SEQ ID NO: 361 - protein, 214 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 363 - protein, 8 aa, Homo sapiens
GYTFSTFG

SEQ ID NO: 364 - protein, 8 aa, Homo sapiens
ISAYNGDT

SEQ ID NO: 365 - protein, 14 aa, Homo sapiens
ARSSGHYYYYGMDV

SEQ ID NO: 366 - protein, 121 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSS

SEQ ID NO: 367 - DNA, 363 bp, Homo sapiens
  source 1..363: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctca

SEQ ID NO: 368 - protein, 451 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - DNA, 1359 bp, Homo sapiens
  source 1..1359: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 370 - protein, 11 aa, Homo sapiens
QSLLHSNEYNY

SEQ ID NO: 371 - skipped

SEQ ID NO: 372 - protein, 9 aa, Homo sapiens
MQSLQTPLT

SEQ ID NO: 373 - protein, 112 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIK

SEQ ID NO: 374 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaa

SEQ ID NO: 375 - protein, 219 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 377 - protein, 8 aa, Homo sapiens
GYTFTSYG

SEQ ID NO: 378 - protein, 8 aa, Homo sapiens
ISAYNGNT

SEQ ID NO: 379 - protein, 17 aa, Homo sapiens
ARSTYFYGSGTLYGMDV

SEQ ID NO: 380 - protein, 124 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSS

SEQ ID NO: 381 - DNA, 372 bp, Homo sapiens
  source 1..372: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctca

SEQ ID NO: 382 - protein, 454 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - DNA, 1368 bp, Homo sapiens
  source 1..1368: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 384 - protein, 11 aa, Homo sapiens
QSLLHSDGYNY

SEQ ID NO: 385 - skipped

SEQ ID NO: 386 - protein, 9 aa, Homo sapiens
MQALQTPLS

SEQ ID NO: 387 - protein, 112 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIK

SEQ ID NO: 388 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaa

SEQ ID NO: 389 - protein, 219 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 390 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 391 - protein, 8 aa, Homo sapiens
GYTFTSYG

SEQ ID NO: 392 - protein, 8 aa, Homo sapiens
ISAYNGNT

SEQ ID NO: 393 - protein, 17 aa, Homo sapiens
ARSTYFYGSGTLYGMDV

SEQ ID NO: 394 - protein, 124 aa, Homo sapiens; identical to SEQ ID NO: 380

SEQ ID NO: 395 - DNA, 372 bp, Homo sapiens; identical to SEQ ID NO: 381
  source 1..372: mol_type = unassigned DNA

SEQ ID NO: 396 - protein, 454 aa, Homo sapiens; identical to SEQ ID NO: 382

SEQ ID NO: 397 - DNA, 1368 bp, Homo sapiens; identical to SEQ ID NO: 383
  source 1..1368: mol_type = unassigned DNA

SEQ ID NO: 398 - protein, 11 aa, Homo sapiens
QSLLHSDGYNC

SEQ ID NO: 399 - skipped

SEQ ID NO: 400 - protein, 9 aa, Homo sapiens
MQALQTPCS

SEQ ID NO: 401 - protein, 112 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIK

SEQ ID NO: 402 - DNA, 336 bp, Homo sapiens; identical to SEQ ID NO: 388
  source 1..336: mol_type = unassigned DNA

SEQ ID NO: 403 - protein, 219 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 404 - DNA, 657 bp, Homo sapiens; identical to SEQ ID NO: 390
  source 1..657: mol_type = unassigned DNA

SEQ ID NO: 405 - protein, 8 aa, Homo sapiens
GVTFDDYG

SEQ ID NO: 406 - protein, 8 aa, Homo sapiens
INWNGGDT

SEQ ID NO: 407 - protein, 17 aa, Homo sapiens
ARDFYGSGSYYHVPFDY

SEQ ID NO: 408 - protein, 124 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 409 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 410 - protein, 454 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 412 - protein, 7 aa, Homo sapiens
QSVSRSY

SEQ ID NO: 413 - skipped

SEQ ID NO: 414 - protein, 9 aa, Homo sapiens
HQYDMSPFT

SEQ ID NO: 415 - protein, 108 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 416 - DNA, 324 bp, Homo sapiens
  source 1..324: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaa

SEQ ID NO: 417 - protein, 215 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - DNA, 645 bp, Homo sapiens
  source 1..645: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 419 - protein, 8 aa, Homo sapiens
GLTFDDYG

SEQ ID NO: 420 - protein, 8 aa, Homo sapiens
INWNGDNT

SEQ ID NO: 421 - protein, 17 aa, Homo sapiens
ARDYYGSGSYYNVPFDY

SEQ ID NO: 422 - protein, 124 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSS

SEQ ID NO: 423 - DNA, 372 bp, Homo sapiens
  source 1..372: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctca

SEQ ID NO: 424 - protein, 454 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - DNA, 1368 bp, Homo sapiens
  source 1..1368: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 426 - protein, 7 aa, Homo sapiens
QSVSSSY

SEQ ID NO: 427 - skipped

SEQ ID NO: 428 - protein, 8 aa, Homo sapiens
QQYGSSPF

SEQ ID NO: 429 - protein, 107 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIK

SEQ ID NO: 430 - DNA, 321 bp, Homo sapiens
  source 1..321: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaa

SEQ ID NO: 431 - DNA, 323 bp, Homo sapiens
  source 1..323: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaa

SEQ ID NO: 432 - protein, 214 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 433 - DNA, 642 bp, Homo sapiens
  source 1..642: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 434 - DNA, 644 bp, Homo sapiens
  source 1..644: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 435 - protein, 8 aa, Homo sapiens
GYTFNSYG

SEQ ID NO: 436 - protein, 8 aa, Homo sapiens
ISVHNGNT

SEQ ID NO: 437 - protein, 17 aa, Homo sapiens
ARAGYDILTDFSDAFDI

SEQ ID NO: 438 - protein, 124 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSS

SEQ ID NO: 439 - DNA, 372 bp, Homo sapiens
  source 1..372: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttca

SEQ ID NO: 440 - protein, 454 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - DNA, 1368 bp, Homo sapiens
  source 1..1368: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 442 - protein, 6 aa, Homo sapiens
QNINNF

SEQ ID NO: 443 - skipped

SEQ ID NO: 444 - protein, 8 aa, Homo sapiens
QQSYGIPW

SEQ ID NO: 445 - protein, 106 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIK

SEQ ID NO: 446 - DNA, 318 bp, Homo sapiens
  source 1..318: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaa

SEQ ID NO: 447 - protein, 213 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - DNA, 639 bp, Homo sapiens
  source 1..639: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 449 - protein, 8 aa, Homo sapiens
GFTFSDYF

SEQ ID NO: 450 - protein, 8 aa, Homo sapiens
ISSSGSTI

SEQ ID NO: 451 - protein, 21 aa, Homo sapiens
ARDHYDGSGIYPLYYYYGLDV

SEQ ID NO: 452 - protein, 128 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSS

SEQ ID NO: 453 - DNA, 384 bp, Homo sapiens
  source 1..384: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctca

SEQ ID NO: 454 - protein, 458 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - DNA, 1380 bp, Homo sapiens
  source 1..1380: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 456 - protein, 11 aa, Homo sapiens
QSLLHSNGYNY

SEQ ID NO: 457 - skipped

SEQ ID NO: 458 - protein, 9 aa, Homo sapiens
MQALQTPRS

SEQ ID NO: 459 - protein, 111 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIK

SEQ ID NO: 460 - DNA, 333 bp, Homo sapiens
  source 1..333: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaa

SEQ ID NO: 461 - protein, 218 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - DNA, 654 bp, Homo sapiens
  source 1..654: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 463 - protein, 10 aa, Homo sapiens
GFSLSTTGVG

SEQ ID NO: 464 - protein, 7 aa, Homo sapiens
IYWDDDK

SEQ ID NO: 465 - protein, 16 aa, Homo sapiens
THGYGSASYYHYGMDV

SEQ ID NO: 466 - protein, 124 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 467 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 468 - protein, 454 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 470 - protein, 6 aa, Homo sapiens
QSVTNY

SEQ ID NO: 471 - skipped

SEQ ID NO: 472 - protein, 9 aa, Homo sapiens
QHRSNWPLT

SEQ ID NO: 473 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIK

SEQ ID NO: 474 - DNA, 322 bp, Homo sapiens
  source 1..322: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaac

SEQ ID NO: 475 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 476 - DNA, 643 bp, Homo sapiens
  source 1..643: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaaccgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 477 - protein, 10 aa, Homo sapiens
GFSLSTSGVG

SEQ ID NO: 478 - protein, 7 aa, Homo sapiens
IYWDDDK

SEQ ID NO: 479 - protein, 16 aa, Homo sapiens
THGYGSASYYHYGMDV

SEQ ID NO: 480 - protein, 124 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 481 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 482 - protein, 454 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 484 - protein, 6 aa, Homo sapiens
QSVTNY

SEQ ID NO: 485 - skipped

SEQ ID NO: 486 - protein, 9 aa, Homo sapiens
QQRSNWPLT

SEQ ID NO: 487 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIK

SEQ ID NO: 488 - DNA, 321 bp, Homo sapiens
  source 1..321: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 489 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - DNA, 642 bp, Homo sapiens
  source 1..642: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 491 - protein, 8 aa, Homo sapiens
GFTFSDYY

SEQ ID NO: 492 - protein, 8 aa, Homo sapiens
ISSSGSTI

SEQ ID NO: 493 - protein, 20 aa, Homo sapiens
ARDFYDILTDSPYFYYGVDV

SEQ ID NO: 494 - protein, 127 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSS

SEQ ID NO: 495 - DNA, 381 bp, Homo sapiens
  source 1..381: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctca

SEQ ID NO: 496 - protein, 457 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 497 - DNA, 1377 bp, Homo sapiens
  source 1..1377: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 498 - protein, 11 aa, Homo sapiens
QSLLHSNGYNY

SEQ ID NO: 499 - skipped

SEQ ID NO: 500 - protein, 9 aa, Homo sapiens
MQALQTPRT

SEQ ID NO: 501 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 502 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 503 - protein, 219 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 505 - protein, 239 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGRDYKDDDDKHHHHHH

SEQ ID NO: 506 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 507 - protein, 121 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKF

SEQ ID NO: 508 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 509 - protein, 33 aa, Homo sapiens
KYSSSVHDPNGEYMFMRAVNTAKKSRLTDVTLM

SEQ ID NO: 510 - protein, 128 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 511 - protein, 121 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLK

SEQ ID NO: 512 - protein, 147 aa, Mus musculus
MGWSCIILFLVATATGVHSEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 513 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 514 - protein, 120 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLK

SEQ ID NO: 515 - protein, 240 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWS

SEQ ID NO: 516 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 517 - protein, 113 aa, Homo sapiens
LQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 518 - protein, 113 aa, Homo sapiens
LQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 519 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaa

SEQ ID NO: 520 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 521 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 522 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 523 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 524 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 526 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 527 - DNA, 978 bp, Homo sapiens
  source 1..978: mol_type = unassigned DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 528 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 529 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 530 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 531 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 532 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 533 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 534 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 535 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 536 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 537 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 538 - protein, 106 aa, Homo sapiens; identical to SEQ ID NO: 225

SEQ ID NO: 539 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 215 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 541 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 542 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 217 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 544 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 545 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 546 - protein, 214 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 547 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 548 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 549 - protein, 216 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 550 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 551 - protein, 659 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 552 - protein, 215 aa, Homo sapiens; identical to SEQ ID NO: 540

SEQ ID NO: 553 - protein, 220 aa, Homo sapiens; identical to SEQ ID NO: 541

SEQ ID NO: 554 - protein, 663 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 555 - protein, 217 aa, Homo sapiens; identical to SEQ ID NO: 543

SEQ ID NO: 556 - protein, 214 aa, Homo sapiens; identical to SEQ ID NO: 544

SEQ ID NO: 557 - protein, 659 aa, Homo sapiens; identical to SEQ ID NO: 551

SEQ ID NO: 558 - protein, 215 aa, Homo sapiens; identical to SEQ ID NO: 540

SEQ ID NO: 559 - protein, 220 aa, Homo sapiens; identical to SEQ ID NO: 541

SEQ ID NO: 560 - protein, 663 aa, Homo sapiens; identical to SEQ ID NO: 554

SEQ ID NO: 561 - protein, 217 aa, Homo sapiens; identical to SEQ ID NO: 543

SEQ ID NO: 562 - protein, 214 aa, Homo sapiens; identical to SEQ ID NO: 544

SEQ ID NO: 563 - DNA, 364 bp, Homo sapiens
  source 1..364: mol_type = unassigned DNA
caggttcaactgatgcagtctggaactgaggtgaagaagcctggggcctcagtgaaggtctcctgcaagacttctggttacacctttaccacctatggtatcacttgggtgcgacaggcccctggacaagggcttgagtggatgggatggatcagcgcttacagtggtgacacagactatgcacagaagttccagggcagagtcaccgtgacaacagacacatccacgaacacagcctacatggagttgaggagcctgaaatctgacgacacggccgtgtattattgtgcgagaagtagtggctggccccaccactacggtatggacgtctggggccaagggaccacggtcaccgtctcctcag

SEQ ID NO: 564 - protein, 121 aa, Homo sapiens
QVQLMQSGTEVKKPGASVKVSCKTSGYTFTTYGITWVRQAPGQGLEWMGWISAYSGDTDYAQKFQGRVTVTTDTSTNTAYMELRSLKSDDTAVYYCARSSGWPHHYGMDVWGQGTTVTVSS

SEQ ID NO: 565 - protein, 8 aa, Homo sapiens
GYTFTTYG

SEQ ID NO: 566 - protein, 8 aa, Homo sapiens
ISAYSGDT

SEQ ID NO: 567 - protein, 14 aa, Homo sapiens
ARSSGWPHHYGMDV

SEQ ID NO: 568 - DNA, 373 bp, Homo sapiens
  source 1..373: mol_type = unassigned DNA
caggttcaactggtgcagtctggaggtgaggtgaaaaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtctccatgaccacagacacatccacgagcacagcctacatggagctgaggagcttgagatctgacgacacggccgtgtatttctgtgcgcgatctacgtcttactatggttcggggaccctatacggtatggacgtctggggccaagggaccacggtcaccgtctcctcag

SEQ ID NO: 569 - protein, 124 aa, Homo sapiens
QVQLVQSGGEVKKPGASVKVSCKASGYTFTSYGFSWVRQAPGQGLEWMGWISAYNGNTNYAQKLQGRVSMTTDTSTSTAYMELRSLRSDDTAVYFCARSTSYYGSGTLYGMDVWGQGTTVTVSS

SEQ ID NO: 570 - protein, 17 aa, Homo sapiens
ARSTSYYGSGTLYGMDV

SEQ ID NO: 571 - DNA, 373 bp, Homo sapiens
  source 1..373: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtctccatgaccacagacacatccacgagcacagcctacatggagctgaggagcttgagatctgacgacacggccgtgtattactgtgcgcgatctacgtcttactatggttcggggaccctctacggtatggacgtctggggccaagggaccacggtcaccgtctcctcag

SEQ ID NO: 572 - protein, 124 aa, Homo sapiens
QVQLVQSGGEVKKPGASVKVSCKASGYTFTSYGFSWVRQAPGQGLEWMGWISAYNGNTNYAQKLQGRVSMTTDTSTSTAYMELRSLRSDDTAVYYCARSTSYYGSGTLYGMDVWGQGTTVTVSS

SEQ ID NO: 573 - DNA, 373 bp, Homo sapiens; identical to SEQ ID NO: 568
  source 1..373: mol_type = unassigned DNA

SEQ ID NO: 574 - protein, 124 aa, Homo sapiens; identical to SEQ ID NO: 569

SEQ ID NO: 575 - DNA, 373 bp, Homo sapiens
  source 1..373: mol_type = unassigned DNA
caggttcaactggtgcagtctggaggtgaggtgaaaaagcctcgggcctcagtgaaggtctcctgcaaggcttctggttacacctttaccagctatgtgttcagctgggtgcgacatgccgctggacaaggactagagtggatgggatggatcagcggttacaatggtaacacaaactatgcacagaagctccagtgcggagtctcgatgaccgcagacacatccacgagcacagcctacatggagctgaggagcttgagatctgacgacacggccgtgtatttctgtgcgcgatctacgtcttactatggtgcggggaccctatacggtatggacgtctggggccaagggaccacggtcaccgtctcctcag

SEQ ID NO: 576 - protein, 124 aa, Homo sapiens
QVQLVQSGGEVKKPRASVKVSCKASGYTFTSYVFSWVRHAAGQGLEWMGWISGYNGNTNYAQKLQCGVSMTADTSTSTAYMELRSLRSDDTAVYFCARSTSYYGAGTLYGMDVWGQGTTVTVSS

SEQ ID NO: 577 - protein, 8 aa, Homo sapiens
GYTFTSYV

SEQ ID NO: 578 - protein, 8 aa, Homo sapiens
ISGYNGNT

SEQ ID NO: 579 - protein, 17 aa, Homo sapiens
ARSTSYYGAGTLYGMDV

SEQ ID NO: 580 - DNA, 373 bp, Homo sapiens
  source 1..373: mol_type = unassigned DNA
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcggccgattactatggttcggggagttattataacgtcccctttgactactggggccagggaaccctggtcaccgtctcctcag

SEQ ID NO: 581 - protein, 124 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYYCAADYYGSGSYYNVPFDYWGQGTLVTVSS

SEQ ID NO: 582 - protein, 8 aa, Homo sapiens
GFTFDDYG

SEQ ID NO: 583 - protein, 8 aa, Homo sapiens
INWNGGST

SEQ ID NO: 584 - protein, 17 aa, Homo sapiens
AADYYGSGSYYNVPFDY

SEQ ID NO: 585 - DNA, 373 bp, Homo sapiens
  source 1..373: mol_type = unassigned DNA
gaggtgcagctggtggagtctgggggaggtgtgatacggcctggggggtccctgagactctcctgtgcagcctctggattcacctttgatgattatggcatgagctgggtccgccaagctccagggaaggggctggagtgggtctctggtattaattggattggtgataacacagattatgcagactctgtgaagggccgattcaccatctccagagacaacgccaagaactccctatatctgcaaatgaacagtctgagagccgaggacacggccttgtattactgtgcgagagattactttggttcggggagttattataacgttccctttgactactggggccagggaaccctggtcaccgtctcctcag

SEQ ID NO: 586 - protein, 124 aa, Homo sapiens
EVQLVESGGGVIRPGGSLRLSCAASGFTFDDYGMSWVRQAPGKGLEWVSGINWIGDNTDYADSVKGRFTISRDNAKNSLYLQMNSLRAEDTALYYCARDYFGSGSYYNVPFDYWGQGTLVTVSS

SEQ ID NO: 587 - protein, 8 aa, Homo sapiens
INWIGDNT

SEQ ID NO: 588 - protein, 17 aa, Homo sapiens
ARDYFGSGSYYNVPFDY

SEQ ID NO: 589 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggattcaactatttcgattggtacctgcagaagccaggacagtctccacagctcctgatctttttggtttctaatcgggcctccggggtccctgacaggttcagtggcagtggatcaggcacagattttacactgaaaatcagcagagtggaggctgaggatgttgggatttattactgcatgcaagctctacaaactccgctcactttcggcggagggaccaaggtggagatcaaac

SEQ ID NO: 590 - protein, 112 aa, Homo sapiens
DIVMTQSPLSLPVTPGEPASISCRSSQSLLHSNGFNYFDWYLQKPGQSPQLLIFLVSNRASGVPDRFSGSGSGTDFTLKISRVEAEDVGIYYCMQALQTPLTFGGGTKVEIK

SEQ ID NO: 591 - protein, 11 aa, Homo sapiens
QSLLHSNGFNY

SEQ ID NO: 592 - skipped

SEQ ID NO: 593 - protein, 9 aa, Homo sapiens
MQALQTPLT

SEQ ID NO: 594 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaac

SEQ ID NO: 595 - protein, 112 aa, Homo sapiens
DIVMTQSPLSLPVTPGEPASISCRSSQSLLHSNGYNCLDWYLQKPGQSPQLLIYLGSTRASGFPDRFSGSGSGTDFTLKISRVEAEDVGVYYCMQALQTPCSFGQGTKLEIK

SEQ ID NO: 596 - protein, 11 aa, Homo sapiens
QSLLHSNGYNC

SEQ ID NO: 597 - DNA, 337 bp, Homo sapiens; identical to SEQ ID NO: 594
  source 1..337: mol_type = unassigned DNA

SEQ ID NO: 598 - protein, 112 aa, Homo sapiens; identical to SEQ ID NO: 595

SEQ ID NO: 599 - DNA, 337 bp, Homo sapiens
  source 1..337: mol_type = unassigned DNA
gatattgtgatgactcagtctccactctccctgcccgtcacccctggagagccggcctccatctcctgcaggtctagtcagagcctcctgcatagtaatggatacaactgtttggattggtacctgcagaagccagggcagtctccacagctcctgatctatttgggttctactcgggcctccgggttccctgacaggttcagtggcagtggatcaggcacagattctacactgaaaatcagcagagtggaggctgaggatgttggggtttattactgcatgcaagctctacaaactccgtgcagttttggccaggggaccaagctggagatcaaac

SEQ ID NO: 600 - protein, 112 aa, Homo sapiens
DIVMTQSPLSLPVTPGEPASISCRSSQSLLHSNGYNCLDWYLQKPGQSPQLLIYLGSTRASGFPDRFSGSGSGTDSTLKISRVEAEDVGVYYCMQALQTPCSFGQGTKLEIK

SEQ ID NO: 601 - DNA, 337 bp, Homo sapiens; identical to SEQ ID NO: 594
  source 1..337: mol_type = unassigned DNA

SEQ ID NO: 602 - protein, 112 aa, Homo sapiens; identical to SEQ ID NO: 595

SEQ ID NO: 603 - DNA, 325 bp, Homo sapiens
  source 1..325: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctttcactttcggccctgggaccaaagtggatatcaaac

SEQ ID NO: 604 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 605 - protein, 9 aa, Homo sapiens
QQYGSSPFT

SEQ ID NO: 606 - DNA, 325 bp, Homo sapiens
  source 1..325: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtatattactgtcaccagtatggtaattcaccattcactttcggccctgggaccaaagtggatatcaaac

SEQ ID NO: 607 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCHQYGNSPFTFGPGTKVDIK

SEQ ID NO: 608 - protein, 9 aa, Homo sapiens
HQYGNSPFT

SEQ ID NO: 609 - protein, 11 aa, Homo sapiens
  VAR_SEQ 1: note = X= any amino acid
  VAR_SEQ 5: note = X = any amino acid
  VAR_SEQ 8: note = X =any amino acid
  VAR_SEQ 9: note = X = is either present or absent, and if present, may be any amino acid.
XGSGXYGXXFD

SEQ ID NO: 610 - protein, 478 aa, synthetic construct
  REGION 1..478: note = ICOSL-Fc fusion protein
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 611 - protein, 446 aa, synthetic construct
  REGION 1..446: note = 37A10S713 Heavy Chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 612 - protein, 218 aa, synthetic construct
  REGION 1..218: note = 37A10S713 Light Chain
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 613 - protein, 453 aa, synthetic construct
  REGION 1..453: note = XMAb23104 Heavy Chain
QVQLVQSGAEVKKPGASVKVSCKASGYTFTGYYMHWVRQAPGQGLEWMGWINPHSGETIYAQKFQGRVTMTRDTSISTAYMELSSLRSEDTAVYYCARTYYYDTSGYYHDAFDVWGQGTMVTVSSASTKGPSVFPLAPSSKSTSGGTAALGCLVKDYFPEPVTVSWNSGALTSGVHTFPAVLQSSGLYSLSSVVIVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRWSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 614 - protein, 214 aa, synthetic construct
  REGION 1..214: note = XmAb23104 Light Chain
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 615 - protein, 142 aa, synthetic construct
  REGION 1..142: note = 314.8 mAb Heavy Chain Variable Region
MGWRCIILFLVSTATGVHSQVQLQQPGTELMKPGASVKLSCKASGYTFTTYWMHWVKQRPGQGLEWIGEIDPSDSYVNYNQNFKGKATLTVDKSSSTAYIQLSSLTSEDSAVYFCARSPDYYGTSLAWFDYWGQGTLVTVST

SEQ ID NO: 616 - protein, 132 aa, synthetic construct
  REGION 1..132: note = 314.8 mAb Light Chain Variable Region
MRCLAEFLGLLVLWIPGVIGDIVMTQAAPSVPVTPGESVSISCRSSKSPLHSNGNIYLYWFLQRPGQSPQLLIYRMSNLASGVPDRFSGSGSGTTFTLKISRVEAEDVGVYYCMQHLEYPYTFGGGTKLEIK

SEQ ID NO: 617 - protein, 125 aa, synthetic construct
  REGION 1..125: note = JMab-136 Heavy Chain Variable Region
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 618 - protein, 107 aa, synthetic construct
  REGION 1..107: note = JMab-136 Light Chain Variable Region
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 619 - protein, 450 aa, synthetic construct
  REGION 1..450: note = ICOS.33 IgGlf S267E heavy chain
EVQLVESGGGLVKPGGSLRLSCAASGFTFSDYFMHWVRQAPGKGLEWVGVIDTKSFNYATYYSDLVKGRFTISRDDSKNTLYLQMNSLKTEDTAVYYCTATIAVPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 620 - protein, 213 aa, synthetic construct
  REGION 1..213: note = ICOS.33 IgGlf S267E light chain
DIQMTQSPSSLSASVGDRVTITCQASQDISNYLSWYQQKPGKAPKLLIYYTNLLAEGVPSRFSGSGSGTDFTFTISSLQPEDIATYYCQQYYNYRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0184791 A1
(43) Pub. Date: Jul. 2, 2026

(54) USES OF ANTI-ICOS ANTIBODIES

(71) Applicant: KYMAB LIMITED, Cambridge (GB)

(72) Inventor: Richard Charles Alfred SAINSON, Cambridge (GB)

(21) Appl. No.: 19/395,073

(22) Filed: Nov. 20, 2025

Related U.S. Application Data
(63) Continuation of application No. 17/747,886, filed on May 18, 2022, now abandoned.
(60) Provisional application No. 63/190,016, filed on May 18, 2021.

(51) Int. Cl. C07K 16/28; A61K 39/00; A61P 35/00

(52) U.S. Cl. CPC C07K 16/2818; A61P 35/00; C07K 16/2827; A61K 2039/507; A61K 2039/545

(57) ABSTRACT

Therapeutic use and dosing regimen of anti-ICOS antibodies or antigen-binding fragments thereof for modulating the ratio between regulatory T cells and effector T cells, stimulating the immune system of patients, and/or treating tumours or cancers, as monotherapy or combination therapy, e.g., with anti-PD-L1 antibodies or antigen-binding fragments thereof.

### RELATED APPLICATIONS

This application is a continuation of U.S. patent application Ser. No. 17/747,886, filed May 18, 2022, which claims the benefit of U.S. Provisional Patent Application Ser. No. 63/190,016, filed May 18, 2021, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Mar. 9, 2026, is named 772229_SA9-642CON_ST26.xml and is 667,230 bytes in size.

### 1.2. FIELD OF THE INVENTION

This invention relates to compositions comprising an anti-ICOS antibody (which may comprise a full length antibody or an antigen-binding fragment thereof) for stimulating the mammalian immune response, especially the T cell response. The invention also relates to medical use of such compositions in immuno-oncology, including anti-tumour therapy by promotion of anti-tumour T cell response in a patient, as well as to use of the compositions in other diseases and conditions where it is of therapeutic benefit to modulate the balance between effector T cells and regulatory T cells in favour of effector T cell activity, for example through stimulation of effector T cells and/or through depletion of regulatory T cells. In some embodiments, the invention relates to an anti-ICOS antibody as monotherapy. In other embodiments, the invention relates to an anti-ICOS antibody as part of a combination therapy, e.g., further comprising an anti-PD-L1 antibody (which may comprise a full length antibody or an antigen-binding fragment thereof. The invention also relates to dosing amounts and/or frequencies of an anti-ICOS antibody (as monotherapy or as part of a combination therapy) that are surprisingly effective at stimulating a mammalian immune response, e.g., an anti-tumour T cell response, in a subject.

### 1.3. BACKGROUND

ICOS (Inducible T cell Co-Stimulator) is a member of the CD28 gene family involved in regulating immune responses, in particular humoral immune responses, first identified in 1999 [1]. It is a 55 kDa transmembrane protein, existing as a disulphide linked homodimer with two differentially glycosylated subunits. ICOS is exclusively expressed on T lymphocytes, and is found on a variety of T cell subsets. It is present at low levels on naïve T lymphocytes but its expression is rapidly induced upon immune activation, being upregulated in response to pro-inflammatory stimuli such as on engagement of TCR and co-stimulation with CD28 [2, 3]. ICOS plays a role in the late phase of T cell activation, memory T cell formation and importantly in the regulation of humoral responses through T cell dependent B cell responses [4, 5]. Intracellularly, ICOS binds PI3K and activates the kinases phophoinositide-dependent kinase 1 (PDK1) and protein kinase B (PKB). Activation of ICOS prevents cell death and upregulates cellular metabolism. In the absence of ICOS (ICOS knock-out) or in the presence of anti-ICOS neutralising antibodies there would be a suppression of pro-inflammatory responses. 1.1, REFERENCES¹ Hutloff A, et al. ICOS is an inducible T-cell co-stimulator structurally and functionally related to CD28. Nature. 1999 Jan. 21; 397(6716):263-6.² Beier K C, et al. Induction, binding specificity and function of human ICOS. Eur J Immunol. 2000 December; 30(12):3707-17.³ Coyle A J, et al. The CD28-related molecule ICOS is required for effective T cell-dependent immune responses. Immunity. 2000 July; 13(1):95-105.⁴ Dong C, et al. ICOS co-stimulatory receptor is essential for T-cell activation and function. Nature. 2001 Jan. 4; 409(6816):97-101.⁵ Mak T W, et al., Costimulation through the inducible costimulator ligand is essential for both T helper and B cell functions in T cell-dependent B cell responses. Nat Immunol. 2003 August; 4(8):765-72.

ICOS binds to ICOS ligand (ICOSL) expressed on B-cells and antigen presenting cells (APC) [6, 7]. As a co-stimulatory molecule it serves to regulate TCR mediated immune responses and antibody responses to antigen. The expression of ICOS on T regulatory cells may be important, as it has been suggested that this cell type plays a negative role in immunosurveillance of cancer cells—there is emerging evidence for this in ovarian cancer [8]. Importantly, ICOS expression has been reported to be higher on intratumoural regulatory T cells (TRegs) compared with CD4+ and CD8+ effector cells that are present in the tumour microenvironment. Depletion of TRegs using antibodies with Fc-mediated cellular effector function has demonstrated strong anti-tumour efficacy in a pre-clinical model [9]. Mounting evidence implicates ICOS in an anti-tumour effect in both animal models as well as patients treated with immune-checkpoint inhibitors. In mice deficient in ICOS or ICOSL the anti-tumor effect of anti-CTLA4 therapy is diminished [10] while in normal mice ICOS ligand increases the effectiveness of anti-CTLA4 treatment in melanoma and prostate cancer [11]. Furthermore, in humans a retrospective study of advanced melanoma patients showed increased levels of ICOS following ipilimumab (anti-CTLA4) treatment [12]. In addition, ICOS expression is upregulated in bladder cancer patients treated with anti-CTLA4 [13]. It has also been observed that in cancer patients treated with anti-CTLA4 therapy the bulk of tumour specific IFN□ producing CD4 T-cells are ICOS positive while sustained elevation of ICOS positive CD4 T cells correlates with survival [Error! Bookmark not defined., Error! Bookmark not defined., 14]. ⁶ Swallow M M, Wallin J J, Sha W C. B7h, a novel costimulatory homolog of B7.1 and B7.2, is induced by TNFalpha. Immunity. 1999 October; 11(4):423-32.⁷ Wang S, et al. Costimulation of T cells by B7-H2, a B7-like molecule that binds ICOS. Blood. 2000 Oct. 15; 96(8):2808-13.⁸ Conrad C, Gilliet M. Plasmacytoid dendritic cells and regulatory T cells in the tumor microenvironment: A dangerous liaison. Oncoimmunology. 2013 May 1; 2(5):e2388.⁹ Simpson et al., Fc-dependent depletion of tumor-infiltrating regulatory T cells co-defines the efficacy of anti-CTLA-4 therapy against melanoma. J. Exp. Med. 210(9):1695-1710 2013¹⁰ Fu T, He Q, Sharma P. The ICOS/ICOSL pathway is required for optimal antitumor responses mediated by anti-CTLA-4 therapy. Cancer Res. 2011 Aug. 15; 71(16):5445-54.¹¹ Fan X, Quezada S A, Sepulveda M A, Sharma P, Allison J P. Engagement of the ICOS pathway markedly enhances efficacy of CTLA-4 blockade in cancer immunotherapy. J Exp Med. 2014 Apr. 7; 211(4):715-25.¹² Carthon, B. C., et al. Preoperative CTLA-4 blockade: Tolerability and immune monitoring in the setting of a presurgical clinical trial. Clin. Cancer Res. 16:2861-2871.¹³ Liakou C I, et al. CTLA-4 blockade increases IFNgamma-producing CD4+ICOShi cells to shift the ratio of effector to regulatory T cells in cancer patients. Proc Natl Acad Sci USA. 2008 Sep. 30; 105(39):14987-92.¹⁴ Vonderheide, R. H., et al. 2010. Tremelimumab in combination with exemestane in patients with advanced breast cancer and treatment-associated modulation of inducible costimulator expression on patient T cells. Clin. Cancer Res. 16:3485-3494.

WO2016/120789 described anti-ICOS antibodies and proposed their use for activating T cells and for treating cancer, infectious disease and/or sepsis. A number of murine anti-ICOS antibodies were generated, of which a sub-set were reported to be agonists of the human ICOS receptor. The antibody “422.2” was selected as the lead anti-ICOS antibody and was humanised to produce a human “IgG4PE” antibody designated “H2L5”. H2L5 was reported to have an affinity of 1.34 nM for human ICOS and 0.95 nM for cynomolgus ICOS, to induce cytokine production in T cells, and to upregulate T cell activation markers in conjunction with CD3 stimulation. However, mice bearing implanted human melanoma cells were reported to show only minimal tumour growth delay or increase in survival when treated with H2L5 hIgG4PE, compared with control treated group. The antibody also failed to produce significant further inhibition of tumour growth in combination experiments with ipilimumab (anti-CTLA-4) or pembrolizumab (anti-PD-1), compared with ipilimumab or pembrolizumab monotherapy. Finally, In mice bearing implanted colon cancer cells (CT26), low doses of a mouse cross reactive surrogate of H2L5 in combination with a mouse surrogate of ipilimumab or pembrolizumab only mildly improved overall survival compared with anti-CTLA4 and anti-PD1 therapy alone. A similar lack of strong therapeutic benefit was shown in mice bearing implanted EMT6 cells.

WO2016/154177 described further examples of anti-ICOS antibodies. These antibodies were reported to be agonists of CD4+ T cells, including effector CD8+ T cells (TEff), and to deplete T regulator cells (TRegs). Selective effects of the antibodies on TEff vs TReg cells were described, whereby the antibodies could preferentially deplete TRegs while having minimal effect on TEffs that express a lower level of ICOS. The anti-ICOS antibodies were proposed for use in treating cancer, and combination therapy with anti-PD-1 or anti-PD-L1 antibodies was described.

### 1.4. SUMMARY OF THE INVENTION

An antibody to ICOS that acts to increase effector T cell activity represents a therapeutic approach in immunooncology and in other medical contexts where a CD8+ T cell response is beneficial, including various diseases and conditions and in vaccination regimens. In many diseases and conditions involving an immune component, a balance exists between effector T cells (TEff) which exert the CD8+ T cell immune response, and regulatory T cells (TReg) which suppress that immune response by downregulating TEffs. The present invention relates to antibodies that modulate this TEff/TReg balance in favour of effector T cell activity. Antibodies that trigger the depletion of ICOS highly positive regulatory T cells would relieve the suppression of TEffs, and thus have a net effect of promoting the effector T cell response. An additional or complementary mechanism for an anti-ICOS antibody is via agonistic activity at the ICOS receptor level, to stimulate the effector T cell response.

The relative expression of ICOS on effector T cells (TEff) compared with regulatory T cells (TReg), and the relative activities of these cell populations, will influence the overall effect of an anti-ICOS antibody in vivo. An envisaged mode of action combines agonism of effector T cells with depletion of ICOS positive regulatory T cells. Differential and even opposing effects on these two different T cell populations may be achievable due to their different levels of ICOS expression. Dual-engineering of the variable and constant regions respectively of an anti-ICOS antibody can provide a molecule that exerts a net positive effect on effector T cell response by affecting the CD8/TReg ratio. An antigen-binding domain of an agonist antibody, which activates the ICOS receptor, may be combined with an antibody constant (Fc) region that promotes downregulation and/or clearance of highly expressing cells to which the antibody is bound. An effector positive constant region may be used to recruit cellular effector functions against the target cells (TRegs), e.g., to promote antibody-dependent cell-mediated cytotoxicity (ADCC) or antibody dependent cell phagocytosis (ADCP). The antibody may thus act both to promote effector T cell activation and to downregulate immunosuppressive T Regulatory cells. Since ICOS is more highly expressed on TRegs than on TEffs, a therapeutic balance may be achieved whereby Teff function is promoted while TRegs are depleted, resulting in a net increase in the T cell immune response (e.g, anti-tumour response or other therapeutically beneficial T cell response).

Several pre-clinical and clinical studies have shown a strong positive correlation between high effector T-cell to T-reg cell ratio in the tumour microenvironment (TME) and overall survival. In ovarian cancer patients the ratio of CD8:T-reg cells has been reported to be an indicator of good clinical outcome [15]. A similar observation was made in metastatic melanoma patients after receiving ipilumumab [16]. In pre-clinical studies, it has also been shown that high effector cell:T-reg ratio in TME is associated with anti-tumour response [17]. ¹⁵ Preston C C, et al., The ratios of CD8+ T cells to CD4+CD25+ FOXP3+ and FOXP3− T cells correlate with poor clinical outcome in human serous ovarian cancer. PLoS One November 14; 8(11):e80063.¹⁶ Hodi F S, et al., Immunologic and clinical effects of antibody blockade of cytotoxic T lymphocyte-associated antigen 4 in previously vaccinated cancer patients. PNAS 2008 Feb. 26; 105(8):3005-10¹⁷ Selby, M. J. et al., Anti-CTLA-4 antibodies of IgG2a isotype enhance antitumor activity through reduction of intratumoral regulatory T cells. Cancer immunology research, 1(1):32-42 2013.

This invention provides antibodies that bind human ICOS, including those with efficacy at surprisingly low doses. The antibodies target the ICOS extracellular domain and thereby bind to T cells expressing ICOS. Examples are provided of antibodies that have been designed to have an agonistic effect on ICOS, thus enhancing the function of effector T cells, as indicated by an ability to increase IFNγ expression and secretion. As noted, anti-ICOS antibodies may also be engineered to deplete cells to which they bind, which should have the effect of preferentially downregulating regulatory T cells, lifting the suppressive effect of these cells on the effector T cell response and thus promoting the effector T cell response overall. Regardless of their mechanism of action, it is demonstrated empirically that anti-ICOS antibodies according to the present invention do stimulate T cell response and have anti-tumour effects in vivo, as shown in the Examples. Through selection of appropriate antibody formats such as those including constant regions with a desired level of Fc effector function, or absence of such effector function where appropriate, the anti-ICOS antibodies may be tailored for use in a variety of medical contexts including treatment of diseases and conditions in which an effector T cell response is beneficial and/or where suppression of regulatory T cells is desired.

Exemplary antibodies include STIM001, STIM002, STIM002-B, STIM003, STIM004, STIM005, STIM006, STIM007, STIM008 and STIM009, the sequences of which are set out herein.

In some embodiments, the invention provides a method of treating a disease or condition amenable to therapy by depleting regulatory T cells (Tregs) and/or increasing effector T cell (Teff) response in a subject in need thereof, the method comprising administering to the subject an anti-ICOS antibody or antigen-binding fragment thereof that binds the extracellular domain of human and/or mouse ICOS, wherein the anti-ICOS antibody or antigen-binding fragment thereof is administered to the subject at a dose of about 0.8 mg to 240 mg.

In some embodiments, the anti-ICOS antibody or antigen-binding fragment thereof used in the method of treating a disease or condition amenable to therapy by depleting regulatory T cells (Tregs) and/or increasing effector T cell (Teff) response comprises heavy chain complimentary determining regions (HCDRs) HCDR1, HCDR2, and HCDR3, and light chain complimentary determining regions (LCDRs) LCDR1, LCDR2, and LCDR3, wherein (a) HCDR1, HCDR2, and HCDR3 comprise sequences having at least 85%, 90%, or 95% sequence identity to the amino acid sequences SEQ ID NO: 363, SEQ ID NO: 364, and SEQ ID NO: 365 and LCDR1, LCDR2, and LCDR3 comprise sequences having at least 85%, 90%, or 95% sequence identity to the amino acid sequences SEQ ID NO: 370, SEQ ID NO: 371, SEQ ID NO: 372; (b) HCDR1, HCDR2, and HCDR3 comprise the sequences having at least 85%, 90%, or 95% sequence identity to the amino acid sequences SEQ ID NO: 377, SEQ ID NO: 378, and SEQ ID NO: 379 and LCDR1, LCDR2, and LCDR3 comprise the sequences having at least 85%, 90%, or 95% sequence identity to the amino acid sequences SEQ ID NO: 384, SEQ ID NO: 385, SEQ ID NO: 386; (c) HCDR1, HCDR2, and HCDR3 comprise the sequences having at least 85%, 90%, or 95% sequence identity to the amino acid sequences SEQ ID NO: 391, SEQ ID NO: 392, and SEQ ID NO: 393 and LCDR1, LCDR2, and LCDR3 comprise the sequences having at least 85%, 90%, or 95% sequence identity to the amino acid sequences SEQ ID NO: 398, SEQ ID NO: 399, SEQ ID NO: 400; (d) HCDR1, HCDR2, and HCDR3 comprise the sequences having at least 85%, 90%, or 95% sequence identity to the amino acid sequences SEQ ID NO: 405, SEQ ID NO: 406, and SEQ ID NO: 407 and LCDR1, LCDR2, and LCDR3 comprise the sequences having at least 85%, 90%, or 95% sequence identity to the amino acid sequences SEQ ID NO: 412, SEQ ID NO: 413, SEQ ID NO: 414; (e) HCDR1, HCDR2, and HCDR3 comprise the sequences having at least 85%, 90%, or 95% sequence identity to the amino acid sequences SEQ ID NO: 419, SEQ ID NO: 420, and SEQ ID NO: 421 and LCDR1, LCDR2, and LCDR3 comprise the sequences having at least 85%, 90%, or 95% sequence identity to the amino acid sequences SEQ ID NO: 426, SEQ ID NO: 427, SEQ ID NO: 428; (f) HCDR1, HCDR2, and HCDR3 comprise the sequences having at least 85%, 90%, or 95% sequence identity to the amino acid sequences SEQ ID NO: 435, SEQ ID NO: 436, and SEQ ID NO: 437 and LCDR1, LCDR2, and LCDR3 comprise the sequences having at least 85%, 90%, or 95% sequence identity to the amino acid sequences SEQ ID NO: 442, SEQ ID NO: 443, SEQ ID NO: 444; (g) HCDR1, HCDR2, and HCDR3 comprise the sequences having at least 85%, 90%, or 95% sequence identity to the amino acid sequences SEQ ID NO: 449, SEQ ID NO: 450, and SEQ ID NO: 451 and LCDR1, LCDR2, and LCDR3 comprise the sequences having at least 85%, 90%, or 95% sequence identity to the amino acid sequences SEQ ID NO: 456, SEQ ID NO: 457, SEQ ID NO: 458; (h) HCDR1, HCDR2, and HCDR3 comprise the sequences having at least 85%, 90%, or 95% sequence identity to the amino acid sequences SEQ ID NO: 463, SEQ ID NO: 464, and SEQ ID NO: 465 and LCDR1, LCDR2, and LCDR3 comprise the sequences having at least 85%, 90%, or 95% sequence identity to the amino acid sequences SEQ ID NO: 470, SEQ ID NO: 471, SEQ ID NO: 472; (i) HCDR1, HCDR2, and HCDR3 comprise the sequences having at least 85%, 90%, or 95% sequence identity to the amino acid sequences SEQ ID NO: 477, SEQ ID NO: 478, and SEQ ID NO: 479 and LCDR1, LCDR2, and LCDR3 comprise the sequences having at least 85%, 90%, or 95% sequence identity to the amino acid sequences SEQ ID NO: 484, SEQ ID NO: 485, SEQ ID NO: 486, or (j) HCDR1, HCDR2, and HCDR3 comprise the sequences having at least 85%, 90%, or 95% sequence identity to the amino acid sequences SEQ ID NO: 491, SEQ ID NO: 492, and SEQ ID NO: 493 and LCDR1, LCDR2, and LCDR3 comprise the sequences having at least 85%, 90%, or 95% sequence identity to the amino acid sequences SEQ ID NO: 498, SEQ ID NO: 499, SEQ ID NO: 500. In some embodiments, (a) HCDR1 comprises the amino acid sequence SEQ ID NO: 363, HCDR2 comprises the amino acid sequence SEQ ID NO: 364, HCDR3 comprises the amino acid sequence SEQ ID NO: 365, LCDR1 comprises the amino acid sequence SEQ ID NO: 370, LCDR2 comprises the amino acid sequence SEQ ID NO: 371, and LCDR3 comprises the amino acid sequence SEQ ID NO: 372; (b) HCDR1 comprises the amino acid sequence SEQ ID NO: 377, HCDR2 comprises the amino acid sequence SEQ ID NO: 378, HCDR3 comprises the amino acid sequence SEQ ID NO: 379, LCDR1 comprises the amino acid sequence SEQ ID NO: 384, LCDR2 comprises the amino acid sequence SEQ ID NO: 385, and LCDR3 comprises the amino acid sequence SEQ ID NO: 386; (c) HCDR1 comprises the amino acid sequence SEQ ID NO: 391, HCDR2 comprises the amino acid sequence SEQ ID NO: 392, HCDR3 comprises the amino acid sequence SEQ ID NO: 393, LCDR1 comprises the amino acid sequence SEQ ID NO: 398, LCDR2 comprises the amino acid sequence SEQ ID NO: 399, and LCDR3 comprises the amino acid sequence SEQ ID NO: 400; (d) HCDR1 comprises the amino acid sequence SEQ ID NO: 405, HCDR2 comprises the amino acid sequence SEQ ID NO: 406, HCDR3 comprises the amino acid sequence SEQ ID NO: 407, LCDR1 comprises the amino acid sequence SEQ ID NO: 412, LCDR2 comprises the amino acid sequence SEQ ID NO: 413, and LCDR3 comprises the amino acid sequence SEQ ID NO: 414; (e) HCDR1 comprises the amino acid sequence SEQ ID NO: 419, HCDR2 comprises the amino acid sequence SEQ ID NO: 420, HCDR3 comprises the amino acid sequence SEQ ID NO: 421, LCDR1 comprises the amino acid sequence SEQ ID NO: 426, LCDR2 comprises the amino acid sequence SEQ ID NO: 427, and LCDR3 comprises the amino acid sequence SEQ ID NO: 428; (f) HCDR1 comprises the amino acid sequence SEQ ID NO: 435, HCDR2 comprises the amino acid sequence SEQ ID NO: 436, HCDR3 comprises the amino acid sequence SEQ ID NO: 437, LCDR1 comprises the amino acid sequence SEQ ID NO: 442, LCDR2 comprises the amino acid sequence SEQ ID NO: 443, and LCDR3 comprises the amino acid sequence SEQ ID NO: 444; (g) HCDR1 comprises the amino acid sequence SEQ ID NO: 449, HCDR2 comprises the amino acid sequence SEQ ID NO: 450, HCDR3 comprises the amino acid sequence SEQ ID NO: 451, LCDR1 comprises the amino acid sequence SEQ ID NO: 456, LCDR2 comprises the amino acid sequence SEQ ID NO: 457, and LCDR3 comprises the amino acid sequence SEQ ID NO: 458; (h) HCDR1 comprises the amino acid sequence SEQ ID NO: 463, HCDR2 comprises the amino acid sequence SEQ ID NO: 464, HCDR3 comprises the amino acid sequence SEQ ID NO: 465, LCDR1 comprises the amino acid sequence SEQ ID NO: 470, LCDR2 comprises the amino acid sequence SEQ ID NO: 471, and LCDR3 comprises the amino acid sequence SEQ ID NO: 472; (i) HCDR1 comprises the amino acid sequence SEQ ID NO: 477, HCDR2 comprises the amino acid sequence SEQ ID NO: 478, HCDR3 comprises the amino acid sequence SEQ ID NO: 479, LCDR1 comprises the amino acid sequence SEQ ID NO: 484, LCDR2 comprises the amino acid sequence SEQ ID NO: 485, and LCDR3 comprises the amino acid sequence SEQ ID NO: 486; or (j) HCDR1 comprises the amino acid sequence SEQ ID NO: 491, HCDR2 comprises the amino acid sequence SEQ ID NO: 492, HCDR3 comprises the amino acid sequence SEQ ID NO: 493, LCDR1 comprises the amino acid sequence SEQ ID NO: 498, LCDR2 comprises the amino acid sequence SEQ ID NO: 499, and LCDR3 comprises the amino acid sequence SEQ ID NO: 500.

In another embodiment, the method of treating a disease or condition amenable to therapy by depleting regulatory T cells (Tregs) and/or increasing effector T cell (Teff) response comprises administering to the subject an anti-ICOS antibody or antigen-binding fragment thereof comprising an HCDR1 that comprises the amino acid sequence SEQ ID NO: 405, an HCDR2 that comprises the amino acid sequence SEQ ID NO: 406, an HCDR3 that comprises the amino acid sequence SEQ ID NO: 407, an LCDR1 that comprises the amino acid sequence SEQ ID NO: 412, an LCDR2 that comprises the amino acid sequence SEQ ID NO: 413, and an LCDR3 that comprises the amino acid sequence SEQ ID NO: 414.

In another embodiment, the method of treating a disease or condition amenable to therapy by depleting regulatory T cells (Tregs) and/or increasing effector T cell (Teff) response comprises administering to the subject an anti-ICOS antibody or antigen-binding fragment thereof comprising a heavy chain variable (VH) domain and a light chain variable (VL) domain, wherein (a) the VH domain comprises a sequence having at least 85%, 90%, or 95% sequence identity to the amino acid sequence SEQ ID NO: 366 and the VL domain comprises a sequence having at least 85%, 90%, or 95% sequence identity to the amino acid sequence SEQ ID NO: 373; (b) the VH domain comprises a sequence having at least 85%, 90%, or 95% sequence identity to the amino acid sequence SEQ ID NO: 380 and the VL domain comprises a sequence having at least 85%, 90%, or 95% sequence identity to the amino acid sequence SEQ ID NO: 387; (c) the VH domain comprises a sequence having at least 85%, 90%, or 95% sequence identity to the amino acid sequence SEQ ID NO: 394 and the VL domain comprises a sequence having at least 85%, 90%, or 95% sequence identity to the amino acid sequence SEQ ID NO: 401; (d) the VH domain comprises a sequence having at least 85%, 90%, or 95% sequence identity to the amino acid sequence SEQ ID NO: 408 and VL domain comprises a sequence having at least 85%, 90%, or 95% sequence identity to the amino acid sequence SEQ ID NO: 415; (e) the VH domain comprises a sequence having at least 85%, 90%, or 95% sequence identity to the amino acid sequence SEQ ID NO: 422 and the VL domain comprises a sequence having at least 85%, 90%, or 95% sequence identity to the amino acid sequence SEQ ID NO: 429; (f) the VH domain comprises a sequence having at least 85%, 90%, or 95% sequence identity to the amino acid sequence SEQ ID NO: 438 and the VL domain comprises a sequence having at least 85%, 90%, or 95% sequence identity to the amino acid sequence SEQ ID NO: 445; (g) the VH domain comprises a sequence having at least 85%, 90%, or 95% sequence identity to the amino acid sequence SEQ ID NO: 452 and VL domain comprises a sequence having at least 85%, 90%, or 95% sequence identity to the amino acid sequence SEQ ID NO: 459; (h) the VH domain comprises a sequence having at least 85%, 90%, or 95% sequence identity to the amino acid sequence SEQ ID NO: 467 and the VL domain comprises a sequence having at least 85%, 90%, or 95% sequence identity to the amino acid sequence SEQ ID NO: 473; (i) the VH domain comprises a sequence having at least 85%, 90%, or 95% sequence identity to the amino acid sequence SEQ ID NO 481; and the VL domain comprises a sequence having at least 85%, 90%, or 95% sequence identity to the amino acid sequence SEQ ID NO: 488; or (j) the VH domain comprises a sequence having at least 85%, 90%, or 95% sequence identity to the amino acid sequence SEQ ID NO: 494 and the VL domain comprises a sequence having at least 85%, 90%, or 95% sequence identity to the amino acid sequence SEQ ID NO: 501. In some embodiments, (a) the VH domain comprises the amino acid sequence SEQ ID NO: 366 and the VL domain comprises the amino acid sequence SEQ ID NO: 373; (b) the VH domain comprises the amino acid sequence SEQ ID NO: 380 and the VL domain comprises the amino acid sequence SEQ ID NO: 387; (c) the VH domain comprises the amino acid sequence SEQ ID NO: 394 and the VL domain comprises the amino acid sequence SEQ ID NO: 401; (d) the VH domain comprises the amino acid sequence SEQ ID NO: 408 and the VL domain comprises the amino acid sequence SEQ ID NO: 415; (e) the VH domain comprises the amino acid sequence SEQ ID NO: 422 and the VL domain comprises the amino acid sequence SEQ ID NO: 429; (f) the VH domain comprises the amino acid sequence SEQ ID NO: 438 and the VL domain comprises the amino acid sequence SEQ ID NO: 445; (g) the VH domain comprises the amino acid sequence SEQ ID NO: 452 and the VL domain comprises the amino acid sequence SEQ ID NO: 459; (h) the VH domain comprises the amino acid sequence SEQ ID NO: 467 and the VL domain comprises the amino acid sequence SEQ ID NO: 473; (i) the VH domain comprises the amino acid sequence SEQ ID NO: 480 and the VL domain comprises the amino acid sequence SEQ ID NO: 487; or (j) the VH domain comprises the amino acid sequence SEQ ID NO: 494 and the VL domain comprises the amino acid sequence SEQ ID NO: 501.

In another embodiment, the method of treating a disease or condition amenable to therapy by depleting regulatory T cells (Tregs) and/or increasing effector T cell (Teff) response comprises administering to the subject an anti-ICOS antibody or antigen-binding fragment thereof comprising a VH domain comprising a sequence having at least 95% sequence identity to SEQ ID NO: 408 and a VL domain comprising a sequence having at least 95% sequence identity to SEQ ID NO: 415. In some embodiments, the VH domain comprises the amino acid sequence SEQ ID NO: 408 and the VL domain comprises the amino acid sequence SEQ ID NO: 415.

In another embodiment, the method of treating a disease or condition amenable to therapy by depleting regulatory T cells (Tregs) and/or increasing effector T cell (Teff) response comprises administering to the subject an anti-ICOS antibody or antigen-binding fragment thereof comprising a heavy chain and a light chain, wherein (a) the heavy chain comprises a sequence having at least 85%, 90%, or 95% sequence identity to the amino acid sequence SEQ ID NO: 368 and the light chain comprises a sequence having at least 85%, 90%, or 95% sequence identity to the amino acid sequence SEQ ID NO: 375; (b) the heavy chain comprises a sequence having at least 85%, 90%, or 95% sequence identity to the amino acid sequence SEQ ID NO: 385 and the light chain comprises a sequence having at least 85%, 90%, or 95% sequence identity to the amino acid sequence SEQ ID NO: 389; (c) the heavy chain comprises a sequence having at least 85%, 90%, or 95% sequence identity to the amino acid sequence SEQ ID NO: 396 and the light chain comprises a sequence having at least 85%, 90%, or 95% sequence identity to the amino acid sequence SEQ ID NO: 403; (d) the heavy chain comprises a sequence having at least 85%, 90%, or 95% sequence identity to the amino acid sequence SEQ ID NO: 410 and the light chain comprises a sequence having at least 85%, 90%, or 95% sequence identity to the amino acid sequence SEQ ID NO: 417; (e) the heavy chain comprises a sequence having at least 85%, 90%, or 95% sequence identity to the amino acid sequence SEQ ID NO: 424 and the light chain comprises a sequence having at least 85%, 90%, or 95% sequence identity to the amino acid sequence SEQ ID NO: 432; (f) the heavy chain comprises a sequence having at least 85%, 90%, or 95% sequence identity to the amino acid sequence SEQ ID NO: 440 and the light chain comprises a sequence having at least 85%, 90%, or 95% sequence identity to the amino acid sequence SEQ ID NO: 447; (g) the heavy chain comprises a sequence having at least 85%, 90%, or 95% sequence identity to the amino acid sequence SEQ ID NO: 454 and the light chain comprises a sequence having at least 85%, 90%, or 95% sequence identity to the amino acid sequence SEQ ID NO: 461; (h) the heavy chain comprises a sequence having at least 85%, 90%, or 95% sequence identity to the amino acid sequence SEQ ID NO: 468 and the light chain comprises a sequence having at least 85%, 90%, or 95% sequence identity to the amino acid sequence SEQ ID NO: 475; (i) the heavy chain comprises a sequence having at least 85%, 90%, or 95% sequence identity to the amino acid sequence SEQ ID NO: 482 and the light chain comprises a sequence having at least 85%, 90%, or 95% sequence identity to the amino acid sequence SEQ ID NO: 489; or (j) the heavy chain comprises a sequence having at least 85%, 90%, or 95% sequence identity to the amino acid sequence SEQ ID NO: 496 and the light chain comprises a sequence having at least 85%, 90%, or 95% sequence identity to the amino acid sequence SEQ ID NO: 503. In some embodiments, (a) the heavy chain comprises the amino acid sequence SEQ ID NO: 368 and the light chain comprises the amino acid sequence SEQ ID NO: 375; (b) the heavy chain comprises the amino acid sequence SEQ ID NO: 382 and the light chain comprises the amino acid sequence SEQ ID NO: 389; (c) the heavy chain comprises the amino acid sequence SEQ ID NO: 396 and the light chain comprises the amino acid sequence SEQ ID NO: 403; (d) the heavy chain comprises the amino acid sequence SEQ ID NO: 410 and the light chain comprises the amino acid sequence SEQ ID NO: 417; (e) the heavy chain comprises the amino acid sequence SEQ ID NO: 424 and the light chain comprises the amino acid sequence SEQ ID NO: 432; (f) the heavy chain comprises the amino acid sequence SEQ ID NO: 440 and the light chain comprises the amino acid sequence SEQ ID NO: 447; (g) the heavy chain comprises the amino acid sequence SEQ ID NO: 454 and the light chain comprises the amino acid sequence SEQ ID NO: 461; (h) the heavy chain comprises the amino acid sequence SEQ ID NO: 468 and the light chain comprises the amino acid sequence SEQ ID NO: 475; (i) the heavy chain comprises the amino acid sequence SEQ ID NO: 482 and the light chain comprises the amino acid sequence SEQ ID NO: 489; or (j) the heavy chain comprises the amino acid sequence SEQ ID NO: 496 and the light chain comprises the amino acid sequence SEQ ID NO: 503.

In another embodiment, the method of treating a disease or condition amenable to therapy by depleting regulatory T cells (Tregs) and/or increasing effector T cell (Teff) response comprises administering to the subject an anti-ICOS antibody or antigen-binding fragment thereof comprising a heavy chain comprising a sequence having at least 95% sequence identity to SEQ ID NO: 410 and a light chain comprising a sequence having at least 95% sequence identity to SEQ ID NO: 417. In some embodiments, the heavy chain comprises the amino acid sequence SEQ ID NO: 410 and the light chain comprises the amino acid sequence SEQ ID NO: 417.

In another embodiment, the method comprises administering an anti-ICOS antibody that is a human IgG1 antibody.

In another embodiment, the method comprises administering KY1044.

In another embodiment, the method comprises administering the anti-ICOS antibody or antigen-binding fragment thereof (e.g., KY1044) to the subject at a dose of about 0.5 mg to about 10 mg. In some embodiments, the method comprises administering the anti-ICOS antibody or antigen-binding fragment thereof (e.g., KY1044) to the subject at a dose of about 0.8 mg to about 8 mg. In some embodiments, the method comprises administering the anti-ICOS antibody or antigen-binding fragment thereof (e.g., KY1044) to the subject at a dose of less than about 8 mg (e.g., at a dose of 7.5 mg or less, at a dose of 7 mg or less). In some embodiments, the method comprises administering the anti-ICOS antibody or antigen-binding fragment thereof (e.g., KY1044) to the subject at a dose of about 0.8 mg to about 2.4 mg. In some embodiments, the method comprises administering the anti-ICOS antibody or antigen-binding fragment thereof (e.g., KY1044) to the subject at a dose of about 2.4 mg to about 8 mg.

In another embodiment, the method comprises administering the anti-ICOS antibody or antigen-binding fragment thereof (e.g., KY1044) to the subject at a dose of about 0.8 mg. In some embodiments, the method comprises administering the anti-ICOS antibody or antigen-binding fragment thereof (e.g., KY1044) to the subject at a dose of about 2.4 mg. In some embodiments, the method comprises administering the anti-ICOS antibody or antigen-binding fragment thereof (e.g., KY1044) to the subject at a dose of about 8 mg.

In another embodiment, the anti-ICOS antibody or antigen-binding fragment thereof (e.g., KY1044) is administered to the subject every 2-6 weeks, e.g., every 2 weeks, every 3 weeks, every 4 weeks, every 5 weeks, or every 6 weeks. In some embodiments, the anti-ICOS antibody or antigen-binding fragment thereof (e.g., KY1044) is administered every 3 weeks. In some embodiment the anti-ICOS antibody or antigen-binding fragment thereof (e.g., KY1044) is administered every 6 weeks. In some embodiment the anti-ICOS antibody or antigen-binding fragment thereof (e.g., KY1044) is administered monthly.

In another embodiment, the anti-ICOS antibody or antigen-binding fragment thereof (e.g., KY1044) is administered once. In some embodiments, the anti-ICOS antibody or antigen-binding fragment thereof (e.g., KY1044) is administered more than once. In some embodiments, the anti-ICOS antibody or antigen-binding fragment thereof (e.g., KY1044) is administered for at least 6 months, e.g., for 6 months, 12 months, or more than 12 months.

In another embodiment, the anti-ICOS antibody or antigen-binding fragment thereof (e.g., KY1044) is administered as a monotherapy. In some embodiments, the anti-ICOS antibody or antigen-binding fragment thereof (e.g., KY1044) is administered in a combination therapy. For instance, in some embodiments the method of treating a disease or condition amenable to therapy by depleting regulatory T cells (Tregs) and/or increasing effector T cell (Teff) response further comprises administering to the subject a second therapeutic agent.

In another embodiment, the second therapeutic comprises an anti-PD-L1 antibody or antigen-binding fragment thereof. In some embodiments, the anti-PD-L1 antibody is atezolizumab. In some embodiments, the anti-PD-L1 antibody or antigen-binding fragment thereof (e.g., atezolizumab) is administered to the subject at a dose of about 1200 mg.

In another embodiment, the anti-PD-L1 antibody or antigen-binding fragment thereof (e.g., atezolizumab) is administered to the subject every 2-6 weeks, e.g., every 2 weeks, every 3 weeks, every 4 weeks, every 5 weeks, or every 6 weeks. In some embodiments, the anti-PD-L1 antibody or antigen-binding fragment thereof (e.g., atezolizumab) is administered every 3 weeks. In some embodiments, the anti-PD-L1 antibody or antigen-binding fragment thereof (e.g., atezolizumab) is administered every 6 weeks. In some embodiments, the anti-PD-L1 antibody or antigen-binding fragment thereof (e.g., atezolizumab) is administered monthly.

In another embodiment, the anti-PD-L1 antibody or antigen-binding fragment thereof (e.g., atezolizumab) is administered once. In some embodiments, the anti-PD-L1 antibody or antigen-binding fragment thereof (e.g., atezolizumab) is administered more than once. In some embodiments, the anti-PD-L1 antibody or antigen-binding fragment thereof (e.g., atezolizumab) is administered for at least 6 months, e.g., for 6 months, 12 months, or more than 12 months. In another embodiment, the anti-PD-L1 antibody or antigen-binding fragment thereof is co-administered to the subject with the anti-ICOS antibody or antigen-binding fragment thereof every 3 weeks.

In another embodiment, the anti-PD-L1 antibody or antigen-binding fragment thereof (e.g., atezolizumab) is administered to the subject in alternating doses with the anti-ICOS antibody or antigen-binding fragment thereof (e.g., KY1044), e.g., wherein the anti-PD-L1 antibody or antigen-binding fragment thereof is administered every 3 weeks and the anti-ICOS antibody or antigen-binding fragment thereof is administered every 6 weeks.

In another embodiment, the method comprises treating a tumour. In some embodiments, the method comprises treating a cancer. In some embodiments, the cancer comprises an advanced and/or metastatic cancer. In some embodiments, the cancer comprises triple negative breast cancer, head and neck squamous cell carcinoma, penile cancer, pancreatic cancer, non-small cell lung cancer, hepatocellular carcinoma, esophageal cancer, gastric cancer, melanoma, renal cell carcinoma, and/or cervical cancer.

Pharmaceutical compositions comprising the antibodies are also provided.

An ICOS knock out animal was used for generating cross-reactive antibodies. Notably, strong titres were obtained in ICOS knock out mice, and highly functional antibodies were isolated from among the antibody repertoire, including desirable cross-reactive antibodies. See WO 2018/029474 A2 (hereby incorporated by reference in its entirety).

Exemplary embodiments of the invention are set out in the drawings, the description below, and in the appended claims.

### 1.5. BRIEF DESCRIPTION OF THE DRAWINGS

Certain aspects and embodiments of the invention will now be described in more detail with reference to the accompanying drawings.

FIG. 1, FIG. 2, FIG. 3, FIG. 4: Graphs showing volumes of A20 tumours over time in mice for the study described in Example 1. Each treatment group is represented by a spider plot showing tumour size in individual animals, n=8 per group. For each group, the number of animals with no sign of tumour (indicating cured of disease) is indicated on the bottom left of the graph. Dosing was performed on days 8, 11, 15, 18, 22, 25 and 29 post tumour cell implantation and the dosing time is indicated by the grey shaded area. Compared with the control group (FIG. 1) and the anti-PD-L1 treatment group (FIG. 2), the STIM001 mIgG2a (FIG. 3) and STIM003 mIgG2a (FIG. 4) treatment groups showed significant inhibition of A20 tumour growth.

FIG. 5: STIM002 VH (top) (SEQ ID NO: 380) and VL (bottom) (SEQ ID NO: 387) domain amino acid sequences, showing residues that differ in the corresponding sequences of STIM001 (VH: SEQ ID NO: 366; VL: SEQ ID NO: 373), STIM002B (VH: SEQ ID NO: 394; VL: SEQ ID NO: 401) and related antibodies CL-61091 (VH: SEQ ID NO: 564; VL: SEQ ID NO: 590), CL-64536 (VH: SEQ ID NO: 569; VL: SEQ ID NO: 595), CL-64837 (VH: SEQ ID NO: 572; VL: SEQ ID NO: 598), CL-64841 (VH: SEQ ID NO: 574; VL: SEQ ID NO: 600) and CL-64912 (VH: SEQ ID NO: 576; VL: SEQ ID NO: 602) and/or in the human germline. Sequence numbering is according to IMGT.

FIG. 6: STIM003 VH (top) (SEQ ID NO: 408) and VL (bottom) (SEQ ID NO: 415) domain amino acid sequences, showing residues that differ in the corresponding sequences of related antibodies CL-71642 (VH: SEQ ID NO: 581; VL: SEQ ID NO: 604) and CL-74570 (VH: SEQ ID NO: 586; VL: SEQ ID NO: 607) and/or in the human germline. Sequence numbering is according to IMGT. The VL domain of antibody CL-71642 obtained from sequencing is shown here without the N terminal residue. From the alignment it can be seen that the full VH domain sequence would comprise an N terminal glutamic acid.

FIG. 7: STIM007 VH (top) (SEQ ID NO: 466) and VL (bottom) (SEQ ID NO: 473) domain amino acid sequences, showing residues that differ in the corresponding sequences of STIM008 (VH: SEQ ID NO: 480; VL: SEQ ID NO: 487) and/or in the human germline. Sequence numbering is according to IMGT.

FIG. 8: Effect of STIM003 (anti-ICOS) and AbW (anti-PD-L1) mIgG2a antibodies in the J558 syngeneic model. Each treatment group is represented by a “spider plot” showing the tumour size of individual animals (n=10 or n=8 per group). STIM003 monotherapy demonstrated some efficacy with 3 of 8 animals cured from their disease. Similarly anti-PDL1 was effective in this model with 6 out of 8 animals cured from their disease by day 37. When combined with anti-PDL1 antibodies, STIM003 mIgG2 fully inhibited tumour growth and improved the survival of treated animals. For each group, the number of animals cured of their disease is indicated on the bottom right of the respective graph. Dosing days are indicated by dotted lines (day 11, 15, 18, 22, 25 and 29).

FIGS. 9A-9B: Quantification of ICOS expression (percentage of positive cells and relative expression/dMFI) on the different TILS cell subtypes in the tumour tissue. (FIG. 9A) The % of immune cell subtypes that are positive for ICOS expression and (FIG. 9B) the ICOS dMFI (relative ICOS expression on ICOS positive cell) of immune cell subtypes of animals treated with saline or anti-PD-L1 or anti-PD-1 surrogate antibodies. The mice were implanted with 100 μl of 1×10⁶ viable cells/ml on day 0 (n=7 or n=8). The animals were dosed i.p with 130 μg of antibody on day 13 and day 15. The tissue samples were isolated and analysed on day 16. CD4+/FOXP3+ cells were only included for the TReg population (right end side graphs) and were excluded from the “effector” CD4 cells (left end side graphs) which are all Foxp3 negative. See Example 3.

FIGS. 10A-10C: Data from A20 in vivo efficacy study. Each treatment group is represented by a “spider plot” showing the tumour size of individual animals (n=10 per group). For each group, the number of animals cured of their disease is indicated on the respective graph. For the multiple dose, dosing was on days 8, 11, 15, 18, 22 and 25, indicated by dotted lines. For the single dose, animals received injection IP only on day 8. (FIG. 10A) Saline; (FIG. 10B) STIM003 mIgG2a multiple dose; (FIG. 10C) STIM003 mIgG2a single dose. See Example 4.

FIG. 11: Kaplan-Meier curves for study reported in Example 4 with STIM003 mIgG2a 60 μg fixed dose. SD=single dose, day 8. MD=multiple doses BIW from day 8.

FIGS. 12A-12H: ICOS expression on major T cells subsets (T-reg [CD4+/FoxP3+], CD4 Eff [CD4+/FoxP3−]cells and CD8+) from CT26 tumour bearing animals (n=4 per time point) dosed with saline. Immune cells phenotyping were conducted on day 1, 2, 3, 4 and 8 post treatment and stained for ICOS expression in all the tissues at all time points. FIGS. 12A-12D showing the percentage of ICOS positive cells at all the time points in four different tissues. FIGS. 12E-12H show the ICOS dMFI (relative expression) all the time points in all the four different tissues. See Example 5.

FIG. 13: FACS analysis demonstrating T-reg depletion in the TME in response to STIM003 mIgG2a antibody. CT-26 tumour bearing animals were treated with a single dose (6, 60 or 200 μg) of STIM003 on day 12 post tumour cell implantation. Tissues (n=4 per time point) where harvested for FACS analysis on day 1, 2, 3, 4 and 8 post treatment. The percentage of T-reg cells (CD4⁺CD25⁺Foxp3⁺) in total tumour (A) and the percentage of T-reg cells in the blood (B) are shown at the different time points. See Example 5.

FIGS. 14A-14D: Increase in CD8:T-reg and CD4 eff:T-reg ratio in response to STIM003 mIgG2a. CT-26 tumour bearing animals received a single dose (6, 60 or 200 μg) of STIM003 mIgG2a on day 12 post tumour cell implantation. Tissues (n=4 per time point) were harvested for FACS analysis on day 1, 2, 3, 4 and 8 post treatment and T eff to T-reg ratios were calculated. (FIG. 14A) & (FIG. 14B), CD8:T-reg ratio in tumour and blood, (FIG. 14C) & (FIG. 14D) CD4-eff:T-reg ratio in tumour and blood. See Example 5.

FIGS. 15A-15H: STIM003 treatment correlates with increased degranulation and Th1 cytokine production by TILs. On day 8 post treatment TILs were isolated and FACS analysis were performed to detect CD107a expression on CD4 and CD8 T cells (FIG. 15A-15B). In parallel, cells from dissociated tumours were rested for 4 hrs in the presence of Brefeldin-A, cells were stained for T cells markers and permeabilised for intracellular staining to detect IFN-7 and TNF-α (FIG. 15C-15H). See Example 5.

FIG. 16A: Evidence of KY1044 target engagement on ICOS positive CD4 memory cells (defined as ICOS+CD3+CD4+FoxP3−CD45RA−). Y-axis measures percentage occupancy on the CD4 memory cells as a function of sample collection date. Blood samples were collected on cycle 1 day 1 (C1D1), cycle 1 day 8 (C1D8), cycle 2 day 1 (C2D1) and cycle 2 day 8 (C2D8). Dose level 1=0.8 mg. Dose level 2=2.4 mg. Lines connect data points for the same patient.

FIG. 16B: Evidence of KY1044 target engagement on ICOS positive CD4 memory cells (defined as ICOS+CD3+CD4+FoxP3−CD45RA−). Y-axis measures percentage occupancy on the CD4 memory cells as a function of sample collection date. Blood samples were collected on cycle 1 day 1 (C1D1), cycle 1 day 8 (C1D8), cycle 2 day 1 (C2D1) and cycle 2 day 8 (C2D8). Dose level 3=8 mg. Dose level 4=24 mg. Dose level 5=80 mg. Dose level 6=240 mg. Lines connect data points for the same patient.

FIG. 17A: KY1044-dependent agonism assessed by measuring circulating cytokine levels. Solid line plot represents mean, shaded area represents 95% confidence interval of the ratio between visits and baseline measurements of GM-CSF for patients treated with KY1044. Light grey data points are from patients (n=27) receiving the lower KY1044 dose levels (0.8 mg and 2.4 mg), which resulted in incomplete receptor occupancy. Dark grey data points are from patients (n=14) receiving the higher KY1044 dose levels (8 mg or above), which resulted in complete receptor occupancy.

FIG. 17B: KY1044-dependent agonism assessed by measuring circulating cytokine levels. Solid line plot represents mean, shaded area represents 95% confidence interval of the ratio between visits and baseline measurements of TNFα for patients treated with KY1044. Light grey data points are from patients (n=27) receiving the lower KY1044 dose levels (0.8 mg and 2.4 mg), which resulted in incomplete receptor occupancy. Dark grey data points are from patients (n=14) receiving the higher KY1044 dose levels (8 mg or above), which resulted in complete receptor occupancy.

FIG. 18A: Interim results of phase I/II clinical trial regarding treatment duration. Median duration of treatment for all enrolled patients was 9 weeks.

FIG. 18B: Interim results of phase I/II clinical trial, showing treatment duration in relation to the therapy regimen and partial or complete receptor occupancy.

FIG. 18C: Interim results of phase I/II clinical trial, showing treatment duration in relation to ICOS receptor occupancy.

### 1.6. DETAILED DESCRIPTION

### 1.6.1. ICOS

Antibodies according to the present invention bind the extracellular domain of human ICOS. Thus, the antibodies bind ICOS-expressing T lymphocytes. “ICOS” or “the ICOS receptor” referred to herein may be human ICOS, unless the context dictates otherwise. Sequences of human, cynomolgus and mouse ICOS are shown in the appended sequence listing, and are available from NCBI as human NCBI ID: NP_036224.1, mouse NCBI ID: NP_059508.2 and cynomolgus GenBank ID: EHH55098.1.

### 1.6.2. Cross-Reactivity

Antibodies according to the present invention are preferably cross-reactive, and may for example bind the extracellular domain of mouse ICOS as well as human ICOS. The antibodies may bind other non-human ICOS, including ICOS of primates such as cynomolgus. An anti-ICOS antibody intended for therapeutic use in humans must bind human ICOS, whereas binding to ICOS of other species would not have direct therapeutic relevance in the human clinical context. Nevertheless, the data herein indicate that antibodies that bind both human and mouse ICOS have properties that render them particularly suitable as agonist and depleting molecules. This may result from one or more particular epitopes being targeted by the cross-reactive antibodies. Regardless of the underlying theory, however, cross-reactive antibodies are of high value and are excellent candidates as therapeutic molecules for pre-clinical and clinical studies.

As explained in the experimental Examples, the STIM antibodies described here were generated using Kymouse™ technology where the mouse had been engineered to lack expression of mouse ICOS (an ICOS knock-out). ICOS knock-out transgenic animals and their use for generating cross-reactive antibodies are further aspects of the present invention.

One way to quantify the extent of species cross-reactivity of an antibody is as the fold-difference in its affinity for antigen or one species compared with antigen of another species, e.g., fold difference in affinity for human ICOS vs mouse ICOS. Affinity may be quantified as K_{D}, referring to the equilibrium dissociation constant of the antibody-antigen reaction as determined by SPR with the antibody in Fab format as described elsewhere herein. A species cross-reactive anti-ICOS antibody may have a fold-difference in affinity for binding human and mouse ICOS that is 30-fold or less, 25-fold or less, 20-fold or less, 15-fold or less, 10-fold or less or 5-fold or less. To put it another way, the K_{D} of binding the extracellular domain of human ICOS may be within 30-fold, 25-fold, 20-fold, 15-fold, 10-fold or 5-fold of the K_{D} of binding the extracellular domain of mouse ICOS. Antibodies can also be considered cross-reactive if the K_{D} for binding antigen of both species meets a threshold value, e.g., if the K_{D} of binding human ICOS and the K_{D} of binding mouse ICOS are both 10 mM or less, preferably 5 mM or less, more preferably 1 mM or less. The K_{D} may be 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

An alternative measure of cross-reactivity for binding human ICOS and mouse ICOS is the ability of an antibody to neutralise ICOS ligand binding to ICOS receptor, such as in an HTRF assay (see Example 8 of U.S. Pat. No. 9,957,323). Examples of species cross-reactive antibodies are provided herein, including STIM001, STIM002, STIM002-B, STIM003, STIM005 and STIM006, each of which was confirmed as neutralising binding of human B7-H2 (ICOS ligand) to human ICOS and neutralising binding of mouse B7-H2 to mouse ICOS in an HTRF assay. Any of these antibodies or their variants may be selected when an antibody cross-reactive for human and mouse ICOS is desired. A species cross-reactive anti-ICOS antibody may have an IC50 for inhibiting binding of human ICOS to human ICOS receptor that is within 25-fold, 20-fold, 15-fold, 10-fold or 5-fold of the IC50 for inhibiting mouse ICOS to mouse ICOS receptor as determined in an HTRF assay. Antibodies can also be considered cross-reactive if the IC50 for inhibiting binding of human ICOS to human ICOS receptor and the IC50 for inhibiting binding of mouse ICOS to mouse ICOS receptor are both 1 mM or less, preferably 0.5 mM or less, e.g., 30 nM or less, 20 nM or less, 10 nM or less. The IC50s may be 5 nM or less, 4 nM or less, 3 nM or less or 2 nM or less. In some cases the IC50s will be at least 0.1 nM, at least 0.5 nM or at least 1 nM.

### 1.6.3. Specificity

Antibodies according to the present invention are preferably specific for ICOS. That is, the antibody binds its epitope on the target protein, ICOS (human ICOS, and preferably mouse and/or cynomolgus ICOS as noted above), but does not show significant binding to molecules that do not present that epitope, including other molecules in the CD28 gene family. An antibody according to the present invention preferably does not bind human CD28. The antibody preferably also does not bind mouse or cynomolgus CD28.

CD28 co-stimulates T cell responses when engaged by its ligands CD80 and CD86 on professional antigen presenting cells in the context of antigen recognition via the TCR. For various in vivo uses of the antibodies described herein, the avoidance of binding to CD28 is considered advantageous. Non-binding of the anti-ICOS antibody to CD28 should allow CD28 to interact with its native ligands and to generate appropriate co-stimulatory signal for T cell activation. Additionally, non-binding of the anti-ICOS antibody to CD28 avoids the risk of superagonism. Over-stimulation of CD28 can induce proliferation in resting T cells without the normal requirement for recognition of a cognate antigen via the TCR, potentially leading to runaway activation of T cells and consequent cytokine-release syndrome, especially in human subjects. The non-recognition of CD28 by antibodies according to the present invention therefore represents an advantage in terms of their safe clinical use in humans.

As discussed elsewhere herein, the present invention extends to multispecific antibodies (e.g., bispecifics). A multispecific (e.g., bispecific) antibody may comprise (i) an antibody antigen binding site for ICOS and (ii) a further antigen binding site (optionally an antibody antigen binding site, as described herein) which recognises another antigen (e.g., PD-L1). Specific binding of individual antigen binding sites may be determined. Thus, antibodies that specifically bind ICOS include antibodies comprising an antigen binding site that specifically binds ICOS, wherein optionally the antigen binding site for ICOS is comprised within an antigen-binding molecule that further includes one or more additional binding sites for one or more other antigens, e.g., a bispecific antibody that binds ICOS and PD-L1.

### 1.6.4. Affinity

The affinity of binding of an antibody to ICOS may be determined. Affinity of an antibody for its antigen may be quantified in terms of the equilibrium dissociation constant K_{D}, the ratio Ka/Kd of the association or on-rate (Ka) and the dissociation or off-rate (kd) of the antibody-antigen interaction. Kd, Ka and Kd for antibody-antigen binding can be measured using surface plasmon resonance (SPR).

An antibody according to the present invention may bind the EC domain of human ICOS with a K_{D} of 10 mM or less, preferably 5 mM or less, more preferably 1 mM or less. The K_{D} may be 50 nM or less, 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less. The K_{D} may be at least 0.001 nM, for example at least 0.01 nM or at least 0.1 nM.

Quantification of affinity may be performed using SPR with the antibody in Fab format. A suitable protocol is as follows:
1. Coupling anti-human (or other antibody constant region species-matched) IgG to a biosensor chip (e.g., GLM chip) such as by primary amine coupling;
2. Exposing the anti-human IgG (or other matched species antibody) to a test antibody, e.g., in Fab format, to capture test antibody on the chip;
3. Passing the test antigen over the chip's capture surface at a range of concentrations, e.g., at 5000 nM, 1000 nM, 200 nM, 40 nM, 8 nM and 2 nM, and at 0 nM (i.e., buffer alone); and
4. Determining the affinity of binding of test antibody to test antigen using SPR at 25° C. Buffer may be at pH 7.6, 150 mM NaCl, 0.05% detergent (e.g., P20) and 3 mM EDTA. Buffer may optionally contain 10 mM HEPES. HBS-EP can be used as running buffer. HBS-EP is available from Teknova Inc (California; catalogue number H8022).

Regeneration of the capture surface can be carried out with 10 mM glycine at pH 1.7. This removes the captured antibody and allows the surface to be used for another interaction. The binding data can be fitted to 1:1 model inherent using standard techniques, e.g., using a model inherent to the ProteOn XPR36™ analysis software.

A variety of SPR instruments are known, such as Biacore™, ProteOn XPR36™ (Bio-Rad®), and KinExA® (Sapidyne Instruments, Inc).

As described, affinity may be determined by SPR with the antibody in Fab format, with the antigen coupled to the chip surface and the test antibody passed over the chip in Fab format in solution, to determine affinity of the monomeric antibody-antigen interaction. Affinity can be determined at any desired pH, e.g., pH 5.5 or pH 7.6, and any desired temperature e.g., 25° C. or 37° C.

Other ways to measure binding of an antibody to ICOS include fluorescence activated cell sorting (FACS), e.g., using cells (e.g., CHO cells) with exogenous surface expression of ICOS or activated primary T cells expressing endogenous levels of ICOS. Antibody binding to ICOS-expressing cells as measured by FACS indicates that the antibody is able to bind the extracellular (EC) domain of ICOS.

### 1.6.5. ICOS Receptor Agonism

The ICOS ligand (ICOSL, also known as B7-H2) is a cell surface expressed molecule that binds to the ICOS receptor [18]. This intercellular ligand-receptor interaction promotes multimerisation of ICOS on the T cell surface, activating the receptor and stimulating downstream signalling in the T cell. In effector T cells, this receptor activation stimulates the effector T cell response. ¹⁸ Chattopadhyay et al., Structural Basis of Inducible Costimulatory Ligand Function: Determination of the Cell Surface Oligomeric State and Functional Mapping of the Receptor Binding Site of the Protein, J. Immunol. 177(6):3920-3929 2006

Anti-ICOS antibodies may act as agonists of ICOS, mimicking and even surpassing this stimulatory effect of the native ICOS ligand on the receptor. Such agonism may result from ability of the antibody to promote multimerisation of ICOS on the T cell. One mechanism for this is where the antibodies form intercellular bridges between ICOS on the T cell surface and receptors on an adjacent cell (e.g., B cell, antigen-presenting cell, or other immune cell), such as Fc receptors. Another mechanism is where antibodies having multiple (e.g., two) antigen-binding sites (e.g., two VH-VL domain pairs) bridge multiple ICOS receptor molecules and so promote multimerisation. A combination of these mechanisms may occur.

Agonism can be tested for in in vitro T cell activation assays, using antibody in soluble form (e.g., in immunoglobulin format or other antibody format comprising two spatially separated antigen-binding sites, e.g., two VH-VL pairs), either including or excluding a cross-linking agent, or using antibody bound to a solid surface to provide a tethered array of antigen-binding sites. Agonism assays may use a human ICOS positive T lymphocyte cell line such as MJ cells (ATCC CRL-8294) as the target T cell for activation in such assays. One or more measures of T cell activation can be determined for a test antibody and compared with a reference molecule or a negative control to determine whether there is a statistically significant (p<0.05) difference in T cell activation effected by the test antibody compared with the reference molecule or the control. One suitable measure of T cell activation is production of cytokines, e.g., IFNγ, TNFα or IL-2. The skilled person will include suitable controls as appropriate, standardising assay conditions between test antibody and control. A suitable negative control is an antibody in the same format (e.g., isotype control) that does not bind ICOS, e.g., an antibody specific for an antigen that is not present in the assay system. A significant difference is observed for test antibody relative to a cognate isotype control within the dynamic range of the assay is indicative that the antibody acts as an agonist of the ICOS receptor in that assay.

An agonist antibody may be defined as one which, when tested in a T cell activation assay:
has a significantly lower EC50 for induction of IFNγ production compared with control antibody;
induces significantly higher maximal IFNγ production compared with control antibody;
has a significantly lower EC50 for induction of IFNγ production compared with ICOSL-Fc;
induces significantly higher maximal IFNγ production compared with ICOSL-Fc;
has a significantly lower EC50 for induction of IFNγ production compared with reference antibody C398.4A; and/or
induces significantly higher maximal IFNγ production compared with reference antibody C398.4A.

Exemplary in vitro T cell assays include the bead-bound assay, the plate-bound assay, and the soluble form assay, as disclosed in Examples 13-15 of U.S. Pat. No. 9,957,323.

A significantly lower or significantly higher value may for example be up to 0.5-fold different, up to 0.75-fold different, up to 2-fold different, up to 3-fold different, up to 4-fold different or up to 5-fold different, compared with the reference or control value.

Thus, in one example, an antibody according to the present invention has a significantly lower, e.g., at least 2-fold lower, EC50 for induction of IFNγ in an MJ cell activation assay using the antibody in bead-bound format, compared with control.

The bead-bound assay uses the antibody (and, for control or reference experiments, the control antibody, reference antibody or ICOSL-Fc) bound to the surface of beads. Magnetic beads may be used, and various kinds are commercially available, e.g., Tosyl-activated DYNABEADS M-450 (DYNAL Inc, 5 Delaware Drive, Lake Success, N.Y. 11042 Prod No. 140.03, 140.04). Beads may be coated, or generally by dissolving the coating material in carbonate buffer (pH 9.6, 0.2 M) or other method known in the art. Use of beads conveniently allows the quantity of protein bound to the bead surface to be determined with a good degree of accuracy. Standard Fc-protein quantification methods can be used for coupled protein quantification on beads. Any suitable method can be used, with reference to a relevant standard within the dynamic range of the assay, including DELFIA, ELISA, or other methods.

Agonism activity of an antibody can also be measured in primary human T lymphocytes ex vivo. The ability of an antibody to induce expression of IFNγ in such T cells is indicative of ICOS agonism. Preferably, an antibody will show significant (p<0.05) induction of IFNγ at 5 μg/ml compared with control antibody in T cell activation assay 1 and/or T cell activation assay 2. As noted above, an anti-ICOS antibody may stimulate T cell activation to a greater degree than ICOS-L or C398.4 in such an assay. Thus, the antibody may show significantly (p<0.05) greater induction of IFNγ at 5 μg/ml compared with the control or reference antibody in T cell activation assay 1 or 2. TNFα or IL-2 induction may be measured as an alternative assay readout.

Agonism of an anti-ICOS antibody may contribute to its ability to change the balance between populations of TReg and TEff cells in vivo, e.g., in a site of pathology such as a tumour microenvironment, in favour of TEff cells. The ability of an antibody to enhance tumour cell killing by activated ICOS-positive effector T cells may be determined, as discussed elsewhere herein.

### ICOS Receptor Agonism and Therapeutic Efficacy at Lower Doses

The present invention is based in part on the discovery that a lower anti-ICOS antibody concentration, resulting from administration of a lower dose to subjects, may improve clinical efficacy compared with a higher anti-ICOS concentration resulting from a higher dose. Surprisingly, as indicated by data presented herein, an anti-ICOS antibody dose that only results in partial receptor/transient occupancy may induce a stronger GM-CSF and TNFα signal after treatment, compared with an anti-ICOS antibody dose that results in full receptor occupancy.

Without being limited by theory, anti-ICOS antibodies such as KY1044 may act as agonists of ICOS by promoting multimerization of ICOS on the T cell. ICOS receptors have a propensity to configure as homodimers. Thus, antibodies having multiple antigen-binding sites to ICOS can bridge multiple ICOS receptor molecules and result in ligand-induced clustering or multimerization. Such ligand bridging is proposed to mediate the avidity effect through increased stability of ligand-receptor interactions.

The multimerization of the ICOS receptor may be dependent in part on the stoichiometric ratio of the antibody concentration and the receptors. For instance, without being limited by theory, if the concentration of antibody is significantly greater than the number of available receptors, then this would favour the formation of isolated receptors bound to two different antibodies and reduce FcγR-dependent stimulation, but if the number of receptors greatly exceeds the number of antibodies present, then ligand bridging would be unlikely to occur and subsequently also lead to reduced FcγR-dependent stimulation. In some embodiments, equal concentration of antibody and receptor are present and promote the formation of multimeric complexes and maximally induce FcγR-dependent stimulation, resulting in a greater release of pro-inflammatory cytokines. Also without being limited by theory, high anti-ICOS antibody opsonization may result in no clustering and/or poor immunological synapse and no co-stimulation, while low anti-ICOS antibody opsonization may improve clustering, resulting in FcγR-dependent co-stimulation.

In some embodiments, the anti-ICOS antibody that is administered in a dose effective to yield partial ICOS receptor/transient occupancy, improve clustering, and/or improve co-stimulation comprises the CDRs of KY1044. In another embodiment, the anti-ICOS antibody comprises heavy and light chain variable domains having at least 85%, 90%, or 95% sequence identity to the heavy and light chain variable domains of KY1044. In some such embodiments, the heavy and light chain variable domains having at least 85%, 90%, or 95% sequence identity to the heavy and light chain variable domains of KY1044 comprise the CDRs of KY1044. In another embodiment, the anti-ICOS antibody comprises the heavy and light chain variable domains of KY1044. In some embodiments, an anti-ICOS antibody dose of about 8 mg yields full ICOS receptor occupancy. Thus, in some embodiments, the anti-ICOS antibody dose effective to yield partial ICOS receptor/transient occupancy, improve clustering, and/or improve co-stimulation is less than about 8 mg, e.g., is about 7 mg, about 6 mg, about 5 mg, about 4 mg, about 3 mg, about 2 mg, about 1 mg, or less than about 1 mg. In one embodiment, the dose of the anti-ICOS antibody is about 2.4 mg. In another embodiment, the dose of the anti-ICOS antibody is about 0.8 mg.

In another embodiment, the anti-ICOS antibody that is administered in a dose effective to yield partial ICOS receptor/transient occupancy, improve clustering, and/or improve co-stimulation comprises heavy and light chains having at least 85%, 90%, or 95% sequence identity to the heavy and light chains of KY1044. In some such embodiments, the heavy and light chains having at least 85%, 90%, or 95% sequence identity to the heavy and light chains of KY1044 comprise the CDRs of KY1044. In another embodiment, the anti-ICOS antibody comprises the heavy and light chains of KY1044. In another embodiment, the anti-ICOS antibody is KY1044. In some embodiments, an anti-ICOS antibody dose of about 8 mg yields full ICOS receptor occupancy. Thus, in some embodiments, the anti-ICOS antibody dose effective to yield partial ICOS receptor/transient occupancy, improve clustering, and/or improve co-stimulation is less than about 8 mg, e.g., is about 7 mg, about 6 mg, about 5 mg, about 4 mg, about 3 mg, about 2 mg, about 1 mg, or less than about 1 mg. In one embodiment, the dose of the anti-ICOS antibody is about 2.4 mg. In another embodiment, the dose of the anti-ICOS antibody is about 0.8 mg.

In some embodiments, intratumoural ICOS+ Treg depletion (decrease in ICOS+FOXP3+ cells) is highest at about 8 mg of the anti-ICOS antibody (e.g., KY1044). In some embodiments, the improvement of the CD8/ICOS+FOXP3+ Treg ratio in the tumour microenvironment yielded by the anti-ICOS antibody plateaus at doses of about 8 mg or higher of the anti-ICOS antibody (e.g., KY1044).

In some embodiments, ICOS agonism is most evident at a dose of an anti-ICOS antibody (e.g., KY1044) lower than about 8 mg. In some embodiments, the agonistic activity of the anti-ICOS antibody (e.g., KY1044) is effective at about 2.4-8 mg. In some embodiments, the agonistic activity of the anti-ICOS antibody (e.g., KY1044) is effective at about 0.8-2.4 mg. In some embodiments, the agonistic activity of the anti-ICOS antibody (e.g., KY1044) is effective at about 2.4 mg. In other embodiments, the agonistic activity of the anti-ICOS antibody (e.g., KY1044) is effective at about 0.8 mg.

### 1.6.6. T Cell Dependent Killing

Effector T cell function can be determined in a biologically relevant context using an in vitro co-culture assay where tumour cells are incubated with relevant immune cells to trigger immune cell-dependent killing, in which the effect of an anti-ICOS antibody on tumour cell killing by TEffs is observed.

The ability of an antibody to enhance tumour cell killing by activated ICOS-positive effector T cells may be determined. An anti-ICOS antibody may stimulate significantly greater (p<0.05) tumour cell killing compared with a control antibody. An anti-ICOS antibody may stimulate similar or greater tumour cell killing in such an assay as compared with a reference molecule such as the ICOS ligand or the C398.4 antibody. A similar degree of tumour cell killing can be represented as the assay readout for the test antibody being less than two-fold different from that for the reference molecule.

### 1.6.7. ICOS Ligand-Receptor Neutralisation Potency

An antibody according to the present invention may be one which inhibits binding of ICOS to its ligand ICOSL.

The degree to which an antibody inhibits binding of the ICOS receptor to its ligand is referred to as its ligand-receptor neutralising potency. Potency is normally expressed as an IC50 value, in pM unless otherwise stated. In ligand-binding studies, IC50 is the concentration that reduces receptor binding by 50% of maximal specific binding level. IC50 may be calculated by plotting % specific receptor binding as a function of the log of the antibody concentration, and using a software program such as Prism (GraphPad) to fit a sigmoidal function to the data to generate IC50 values. Neutralising potency may be determined in an HTRF assay, as disclosed in Example 8 of U.S. Pat. No. 9,957,323.

An IC50 value may represent the mean of a plurality of measurements. Thus, for example, IC50 values may be obtained from the results of triplicate experiments, and a mean IC50 value can then be calculated.

An antibody may have an IC50 of 1 mM or less in a ligand-receptor neutralisation assay, e.g., 0.5 mM or less. The IC50 may be, 30 nM or less, 20 nM or less, 10 nM or less, 5 nM or less, 4 nM or less, 3 nM or less or 2 nM or less. The IC50 may be at least 0.1 nM, at least 0.5 nM or at least 1 nM.

### 1.6.8. Antibodies

As described in the Examples of U.S. Pat. No. 9,957,323, we isolated and characterised antibodies of particular interest, designated STIM001, STIM002, STIM002-B, STIM003, STIM004, STIM005, STIM006, STIM007, STIM008 and STIM009. In various aspects of the invention, unless context dictates otherwise, antibodies may be selected from any of these antibodies, or from the sub-set of STIM001, STIM002, STIM003, STIM004 and STIM005. Sequences of each of these antibodies are provided in the appended sequence listing, wherein for each antibody the following sequences are shown: nucleotide sequence encoding VH domain; amino acid sequence of VH domain; VH CDR1 amino acid sequence, VH CDR2 amino acid sequence; VH CDR3 amino acid sequence; nucleotide sequence encoding VL domain; amino acid sequence of VL domain; VL CDR1 amino acid sequence; VL CDR2 amino acid sequence; and VL CDR3 amino acid sequence, respectively. The present invention encompasses anti-ICOS antibodies having the VH and/or VL domain sequences of all antibodies shown in the appended sequence listing and/or in the drawings, as well as antibodies comprising the HCDRs and/or LCDRs of those antibodies, and optionally having the full heavy chain and/or full light chain amino acid sequence.

STIM001 has a heavy chain variable region (V_{H}) amino acid sequence of Seq ID No:366, comprising the CDRH1 amino acid sequence of Seq ID No:363, the CDRH2 amino acid sequence of Seq ID No:364, and the CDRH3 amino acid sequence of Seq ID No:365. The heavy chain nucleic acid sequence of the VH domain is Seq ID No:367. STIM001 has a light chain variable region (V_{L}) amino acid sequence of Seq ID No:373, comprising the CDRL1 amino acid sequence of Seq ID No:370, the CDRL2 amino acid sequence of Seq ID No:371, and the CDRL3 amino acid sequence of Seq ID No:372. The light chain nucleic acid sequence of the V_{L} domain is Seq ID No:374. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:368 (heavy chain nucleic acid sequence Seq ID No:369). A full length light chain amino acid sequence is Seq ID No:375 (light chain nucleic acid sequence Seq ID No:376).

STIM002 has a heavy chain variable region (V_{H}) amino acid sequence of Seq ID No:380, comprising the CDRH1 amino acid sequence of Seq ID No:377, the CDRH2 amino acid sequence of Seq ID No:378, and the CDRH3 amino acid sequence of Seq ID No:379. The heavy chain nucleic acid sequence of the VH domain is Seq ID No:381. STIM002 has a light chain variable region (V_{L}) amino acid sequence of Seq ID No:387, comprising the CDRL1 amino acid sequence of Seq ID No:384, the CDRL2 amino acid sequence of Seq ID No:385, and the CDRL3 amino acid sequence of Seq ID No:386. The light chain nucleic acid sequence of the V_{L} domain is Seq ID No:388 or Seq ID No:519. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:382 (heavy chain nucleic acid sequence Seq ID No:383). A full length light chain amino acid sequence is Seq ID No:389 (light chain nucleic acid sequence Seq ID No:390 or Seq ID NO:520).

STIM002-B has a heavy chain variable region (VH) amino acid sequence of Seq ID No:394, comprising the CDRH1 amino acid sequence of Seq ID No:391, the CDRH2 amino acid sequence of Seq ID No:392, and the CDRH3 amino acid sequence of Seq ID No:393. The heavy chain nucleic acid sequence of the V_{H} domain is Seq ID No:395. STIM002-B has a light chain variable region (V_{L}) amino acid sequence of Seq ID No:401, comprising the CDRL1 amino acid sequence of Seq ID No:398, the CDRL2 amino acid sequence of Seq ID No:399, and the CDRL3 amino acid sequence of Seq ID No:400. The light chain nucleic acid sequence of the V_{L} domain is Seq ID No:402. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:396 (heavy chain nucleic acid sequence Seq ID No:397). A full length light chain amino acid sequence is Seq ID No:403 (light chain nucleic acid sequence Seq ID No:404).

STIM003, interchangeably referred to herein as KY1044, has a heavy chain variable region (V_{H}) amino acid sequence of Seq ID No:408, comprising the CDRH1 amino acid sequence of Seq ID No:405, the CDRH2 amino acid sequence of Seq ID No:406, and the CDRH3 amino acid sequence of Seq ID No:407. The heavy chain nucleic acid sequence of the V_{H} domain is Seq ID No:409 or Seq ID No:521. STIM003 has a light chain variable region (V_{L}) amino acid sequence of Seq ID No:415, comprising the CDRL1 amino acid sequence of Seq ID No:412, the CDRL2 amino acid sequence of Seq ID No:413, and the CDRL3 amino acid sequence of Seq ID No:414. The light chain nucleic acid sequence of the V_{L} domain is Seq ID No:4416. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:410 (heavy chain nucleic acid sequence Seq ID No:411 or Seq ID No:522). A full length light chain amino acid sequence is Seq ID No:417 (light chain nucleic acid sequence Seq ID No:418).

STIM004 has a heavy chain variable region (V_{H}) amino acid sequence of Seq ID No:422, comprising the CDRH1 amino acid sequence of Seq ID No:419, the CDRH2 amino acid sequence of Seq ID No:420, and the CDRH3 amino acid sequence of Seq ID No:421. The heavy chain nucleic acid sequence of the VH domain is Seq ID No:423. STIM004 has a light chain variable region (V_{L}) amino acid sequence of Seq ID No:429, comprising the CDRL1 amino acid sequence of Seq ID No:426, the CDRL2 amino acid sequence of Seq ID No:427, and the CDRL3 amino acid sequence of Seq ID No:428. The light chain nucleic acid sequence of the V_{L} domain is Seq ID No:430 or Seq ID No:431. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:424 (heavy chain nucleic acid sequence Seq ID No:425). A full length light chain amino acid sequence is Seq ID No:432 (light chain nucleic acid sequence Seq ID No:433 or Seq ID no: 434).

STIM005 has a heavy chain variable region (V_{H}) amino acid sequence of Seq ID No:438, comprising the CDRH1 amino acid sequence of Seq ID No:435, the CDRH2 amino acid sequence of Seq ID No:436, and the CDRH3 amino acid sequence of Seq ID No:437. The heavy chain nucleic acid sequence of the VH domain is Seq ID No:439. STIM005 has a light chain variable region (V_{L}) amino acid sequence of Seq ID No:445, comprising the CDRL1 amino acid sequence of Seq ID No:442, the CDRL2 amino acid sequence of Seq ID No:443, and the CDRL3 amino acid sequence of Seq ID No:444. The light chain nucleic acid sequence of the V_{L} domain is Seq ID No:446. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:440 (heavy chain nucleic acid sequence Seq ID No:441). A full length light chain amino acid sequence is Seq ID No:447 (light chain nucleic acid sequence Seq ID No:448).

STIM006 has a heavy chain variable region (V_{H}) amino acid sequence of Seq ID No:452, comprising the CDRH1 amino acid sequence of Seq ID No:449, the CDRH2 amino acid sequence of Seq ID No:450, and the CDRH3 amino acid sequence of Seq ID No:451. The heavy chain nucleic acid sequence of the VH domain is Seq ID No:453. STIM006 has a light chain variable region (V_{L}) amino acid sequence of Seq ID No:459, comprising the CDRL1 amino acid sequence of Seq ID No:456, the CDRL2 amino acid sequence of Seq ID No:457, and the CDRL3 amino acid sequence of Seq ID No:458. The light chain nucleic acid sequence of the V_{L} domain is Seq ID No:460. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:454 (heavy chain nucleic acid sequence Seq ID No:455). A full length light chain amino acid sequence is Seq ID No:461 (light chain nucleic acid sequence Seq ID No:462).

STIM007 has a heavy chain variable region (V_{H}) amino acid sequence of Seq ID No:466, comprising the CDRH1 amino acid sequence of Seq ID No:463, the CDRH2 amino acid sequence of Seq ID No:464, and the CDRH3 amino acid sequence of Seq ID No:465. The heavy chain nucleic acid sequence of the VH domain is Seq ID No:467. STIM007 has a light chain variable region (V_{L}) amino acid sequence of Seq ID No:473, comprising the CDRL1 amino acid sequence of Seq ID No:470, the CDRL2 amino acid sequence of Seq ID No:471, and the CDRL3 amino acid sequence of Seq ID No:472. The light chain nucleic acid sequence of the V_{L} domain is Seq ID No:474. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:468 (heavy chain nucleic acid sequence Seq ID No:469). A full length light chain amino acid sequence is Seq ID No:475 (light chain nucleic acid sequence Seq ID No:476).

STIM008 has a heavy chain variable region (V_{H}) amino acid sequence of Seq ID No:480, comprising the CDRH1 amino acid sequence of Seq ID No:477, the CDRH2 amino acid sequence of Seq ID No:478, and the CDRH3 amino acid sequence of Seq ID No:479. The heavy chain nucleic acid sequence of the VH domain is Seq ID No:481. STIM008 has a light chain variable region (V_{L}) amino acid sequence of Seq ID No:487, comprising the CDRL1 amino acid sequence of Seq ID No:484, the CDRL2 amino acid sequence of Seq ID No:485, and the CDRL3 amino acid sequence of Seq ID No:486. The light chain nucleic acid sequence of the V_{L} domain is Seq ID No:488. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:482 (heavy chain nucleic acid sequence Seq ID No:483). A full length light chain amino acid sequence is Seq ID No:489 (light chain nucleic acid sequence Seq ID No:490).

STIM009 has a heavy chain variable region (V_{H}) amino acid sequence of Seq ID No:494, comprising the CDRH1 amino acid sequence of Seq ID No:491, the CDRH2 amino acid sequence of Seq ID No:492, and the CDRH3 amino acid sequence of Seq ID No:493. The heavy chain nucleic acid sequence of the VH domain is Seq ID No:495. STIM009 has a light chain variable region (V_{L}) amino acid sequence of Seq ID No:501, comprising the CDRL1 amino acid sequence of Seq ID No:498, the CDRL2 amino acid sequence of Seq ID No:499, and the CDRL3 amino acid sequence of Seq ID No:500. The light chain nucleic acid sequence of the V_{L} domain is Seq ID No:502. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:496 (heavy chain nucleic acid sequence Seq ID No:497). A full length light chain amino acid sequence is Seq ID No:503 (light chain nucleic acid sequence Seq ID No:504).

Additional exemplary anti-ICOS antibodies include, but are not limited to: 37A10S713 (also referred to as vopratelimab or JTX-2011) (see, e.g., U.S. Pat. Nos. 10,023,635 and 11,292,840; WO2017070423; WO 2016154177); XMAb23104 (also referred to as XmAb104) (see U.S. patent Ser. No. 10/981,992); 314.8 mAb (also referred to as Icos 314-8) (WO2014033327A1, WO2012131004A2; U.S. patent Ser. No. 11/180,556), JMab-136 (also referred to as IC009) (see, e.g., WO2008137915; U.S. Pat. No. 9,193,789, US20110243929A1) and ICOS.33 IgGlf S267E (U.S. patent Ser. No. 10/898,556). Antibodies to ICOS and methods of use in the treatment of disease are also described in WO2019222188A1 and U.S. patent Ser. No. 11/292,840 Antibodies to ICOS are also disclosed in EP1374902, EP1374901, and EP1125585. Agonist antibodies to ICOS are also disclosed in US20210340250A1; WO2018222711A2; WO2021209356A1; WO2016120789; US20160215059A1; and WO2012131004A2.

Sequences of heavy and light chains of 37A10S713 are disclosed as SEQ ID NOs:611-612.

37A10S713 Heavy Chain:
(SEQ ID NO: 611)
EVQLVESGGGLVQPGGSLRLSCAASGFTFSDYWMD
WVRQAPGKGLVWVSNIDEDGSITEYSPFVKGRFTI
SRDNAKNTLYLQMNSLRAEDTAVYYCTRWGRFGFD
SWGQGTLVTVSSASTKGPSVFPLAPSSKSTSGGTA
ALGCLVKDYFPEPVTVSWNSGALTSGVHTFPAVLQ
SSGLYSLSSVVTVPSSSLGTQTYICNVNHKPSNTK
VDKKVEPKSCDKTHTCPPCPAPELLGGPSVFLFPP
KPKDTLMISRTPEVTCVVVDVSHEDPEVKFNWYVD
GVEVHNAKTKPREEQYNSTYRVVSVLTVLHQDWLN
GKEYKCKVSNKALPAPIEKTISKAKGQPREPQVYT
LPPSREEMTKNQVSLTCLVKGFYPSDIAVEWESNG
QPENNYKTTPPVLDSDGSFFLYSKLTVDKSRWQQG
NVFSCSVMHEALHNHYTQKSLSLSPG
37A10S713 Light Chain:
(SEQ ID NO: 612)
DIVMTQSPDSLAVSLGERATINCKSSQSLLSGSFN
YLTWYQQKPGQPPKLLIFYASTRHTGVPDRFSGSG
SGTDFTLTISSLQAEDVAVYYCHHHYNAPPTFGPG
TKVDIKRTVAAPSVFIFPPSDEQLKSGTASVVCLL
NNFYPREAKVQWKVDNALQSGNSQESVTEQDSKDS
TYSLSSTLTLSKADYEKHKVYACEVTHQGLSSPVT
KSFNRGEC

In one embodiment, the ICOS binding protein is vopratelimab. In one embodiment, the ICOS binding protein is JTX-2011.

Sequences of heavy and light chains of XMAb23104 are disclosed as SEQ ID NOS:613-614.

XmAb23104 Heavy Chain:
(SEQ ID NO: 613)
QVQLVQSGAEVKKPGASVKVSCKASGYTFTGYYMH
WVRQAPGQGLEWMGWINPHSGETIYAQKFQGRVTM
TRDTSISTAYMELSSLRSEDTAVYYCARTYYYDTS
GYYHDAFDVWGQGTMVTVSSASTKGPSVFPLAPSS
KSTSGGTAALGCLVKDYFPEPVTVSWNSGALTSGV
HTFPAVLQSSGLYSLSSVVIVPSSSLGTQTYICNV
NHKPSDTKVDKKVEPKSCDKTHTCPPCPAPPVAGP
SVFLFPPKPKDTLMISRTPEVTCVVVDVKHEDPEV
KFNWYVDGVEVHNAKTKPREEEYNSTYRWSVLTVL
HQDWLNGKEYKCKVSNKALPAPIEKTISKAKGQPR
EPQVYTLPPSREEMTKNQVSLTCDVSGFYPSDIAV
EWESDGQPENNYKTTPPVLDSDGSFFLYSKLTVDK
SRWEQGDVFSCSVLHEALHSHYTQKSLSLSPGK
XmAb23104 Light Chain:
(SEQ ID NO: 614)
DIQMTQSPSSVSASVGDRVTITCRASQGISRLLAW
YQQKPGKAPKLLIYVASSLQSGVPSRFSGSGSGTD
FTLTISSLQPEDFATYYCQQANSFPWTFGQGTKVE
IK/RTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC

Sequences of heavy and light chain variable regions of 314.8 mAb are disclosed as SEQ ID NOS:615-616.

314.8 mAb Heavy Chain Variable Region:
(SEQ ID NO: 615)
MGWRCIILFLVSTATGVHSQVQLQQPGTELMKPGA
SVKLSCKASGYTFTTYWMHWVKQRPGQGLEWIGEI
DPSDSYVNYNQNFKGKATLTVDKSSSTAYIQLSSL
TSEDSAVYFCARSPDYYGTSLAWFDYWGQGTLVTV
ST
314.8 mAb Light Chain Variable Region:
(SEQ ID NO: 616)
MRCLAEFLGLLVLWIPGVIGDIVMTQAAPSVPVTP
GESVSISCRSSKSPLHSNGNIYLYWFLQRPGQSPQ
LLIYRMSNLASGVPDRFSGSGSGTTFTLKISRVEA
EDVGVYYCMQHLEYPYTFGGGTKLEIK

Sequences of heavy and light chain variable regions of JMab-136 are disclosed as SEQ ID Nos:617-618.

JMab-136 Heavy Chain Variable Region:
(SEQ ID NO: 617)
QVQLVQSGAEVKKPGASVKVSCKASGYTFTGYYMH
WVRQAPGQGLEWMGWINPHSGGTNYAQKFQGRVTM
TRDTSISTAYMELSRLRSDDTAVYYCARTYYYDSS
GYYHDAFDIWGQGTMVTVSS
JMab-136 Light Chain Variable Region:
(SEQ ID NO: 618)
DIQMTQSPSSVSASVGDRVTITCRASQGISRLLAW
YQQKPGKAPKLLIYVASSLQSGVPSRFSGSGSGTD
FTLTISSLQPEDFATYYCQQANSFPWTFGQGTKVE
IK

Sequences of heavy and light chains of ICOS.33 IgGlf S267E are disclosed as SEQ ID NOs:619-620.

ICOS.33 IgG1f S267E heavy chain:
SEQ ID NO: 619
EVQLVESGGGLVKPGGSLRLSCAASGFTFSDYFMH
WVRQAPGKGLEWVGVIDTKSFNYATYYSDLVKGRF
TISRDDSKNTLYLQMNSLKTEDTAVYYCTATIAVP
YYFDYWGQGTLVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKRVEPKSCDKTHTCPPCPAPELLGGPSVF
LFPPKPKDTLMISRTPEVTCVVVDVEHEDPEVKFN
WYVDGVEVHNAKTKPREEQYNSTYRVVSVLTVLHQ
DWLNGKEYKCKVSNKALPAPIEKTISKAKGQPREP
QVYTLPPSREEMTKNQVSLTCLVKGFYPSDIAVEW
ESNGQPENNYKTTPPVLDSDGSFFLYSKLTVDKSR
WQQGNVFSCSVMHEALHNHYTQKSLSLSPG.
ICOS.33 IgG1f S267E light chain:
SEQ ID NO: 620
DIQMTQSPSSLSASVGDRVTITCQASQDISNYLSW
YQQKPGKAPKLLIYYTNLLAEGVPSRFSGSGSGTD
FTFTISSLQPEDIATYYCQQYYNYRTFGPGTKVDI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC.

The term “antibody” refers to a (full length) antibody as well as to an antigen-binding fragment thereof. Antibodies according to the present invention are immunoglobulins or molecules comprising immunoglobulin domains, whether natural or partly or wholly synthetically produced. Antibodies may be IgG, IgM, IgA, IgD or IgE molecules or antigen-specific (antigen-binding) antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. Antibodies can be humanised using routine technology. The term antibody covers any polypeptide or protein comprising an antibody antigen-binding site. An antigen-binding site (paratope) is the part of an antibody that binds to and is complementary to the epitope of its target antigen (ICOS).

The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The antigen binding site is a polypeptide or domain that comprises one or more CDRs of an antibody and is capable of binding the antigen. For example, the polypeptide comprises a CDR3 (e.g., HCDR3). For example the polypeptide comprises CDRs 1 and 2 (e.g., HCDR1 and 2) or CDRs 1-3 of a variable domain of an antibody (e.g., HCDRs1-3).

An antibody antigen-binding site may be provided by one or more antibody variable domains. In an example, the antibody binding site is provided by a single variable domain, e.g., a heavy chain variable domain (VH domain) or a light chain variable domain (VL domain). In another example, the binding site comprises a VH/VL pair or two or more of such pairs. Thus, an antibody antigen-binding site may comprise a VH and a VL.

The antibody may be a whole immunoglobulin, including constant regions, or may be an antibody fragment, e.g., antigen-binding fragment of an antibody. An antibody fragment is a portion of an intact antibody, for example comprising the antigen binding and/or variable region of the intact antibody. Examples of antibody fragments include:
(i) a Fab fragment, a monovalent fragment consisting of the VL, VH, CL and CH1 domains;
(ii) a F(ab′)2 fragment, a bivalent fragment including two Fab fragments linked by a disulfide bridge at the hinge region;
(iii) an Fd fragment consisting of the VH and CH1 domains;
(iv) an Fv fragment consisting of the VL and VH domains of a single arm of an antibody,
(v) a dAb fragment (Ward et al., (1989) Nature 341:544-546; which is incorporated by reference herein in its entirety), which consists of a VH or VL domain; and
(vi) an isolated complementarity determining region (CDR) that retains specific antigen-binding functionality.

Further examples of antibodies are H2 antibodies that comprise a dimer of a heavy chain (5′-VH-(optional hinge)-CH2-CH3-3′) and are devoid of a light chain.

Single-chain antibodies (e.g., scFv) are a commonly used fragment. Multispecific antibodies may be formed from antibody fragments. An antibody of the invention may employ any such format, as appropriate.

Optionally, the antibody immunoglobulin domains may be fused or conjugated to additional polypeptide sequences and/or to labels, tags, toxins or other molecules. Antibody immunoglobulin domains may be fused or conjugated to one or more different antigen binding regions, providing a molecule that is able to bind a second antigen in addition to ICOS. An antibody of the present invention may be a multispecific antibody, e.g., a bispecific antibody, comprising (i) an antibody antigen binding site for ICOS and (ii) a further antigen binding site (optionally an antibody antigen binding site, as described herein) which recognises another antigen (e.g., PD-L1).

An antibody normally comprises an antibody VH and/or VL domain. Isolated VH and VL domains of antibodies are also part of the invention. The antibody variable domains are the portions of the light and heavy chains of antibodies that include amino acid sequences of complementarity determining regions (CDRs; ie., CDR1, CDR2, and CDR3), and framework regions (FRs). Thus, within each of the VH and VL domains are CDRs and FRs. A VH domain comprises a set of HCDRs, and a VL domain comprises a set of LCDRs. VH refers to the variable domain of the heavy chain. VL refers to the variable domain of the light chain. Each VH and VL is typically composed of three CDRs and four FRs, arranged from amino-terminus to carboxy-terminus in the following order: FR1, CDR1, FR2, CDR2, FR3, CDR3, FR4. According to the methods used in this invention, the amino acid positions assigned to CDRs and FRs may be defined according to Kabat (Sequences of Proteins of Immunological Interest (National Institutes of Health, Bethesda, Md., 1987 and 1991)) or according to IMGT nomenclature. An antibody may comprise an antibody VH domain comprising a VH CDR1, CDR2 and CDR3 and a framework. It may alternatively or also comprise an antibody VL domain comprising a VL CDR1, CDR2 and CDR3 and a framework. Examples of antibody VH and VL domains and CDRs according to the present invention are as listed in the appended sequence listing that forms part of the present disclosure. The CDRs shown in the sequence listing are defined according to the IMGT system [19]. All VH and VL sequences, CDR sequences, sets of CDRs and sets of HCDRs and sets of LCDRs disclosed herein represent aspects and embodiments of the invention. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs. ¹⁹ Lefranc M P, IMGT unique numbering for immunoglobulin and T cell receptor variable domains and Ig superfamily V-like domains, Dev Comp Immunol. 27(1):55-77 2003

An antibody the invention may comprise one or more CDRs as described herein, e.g. a CDR3, and optionally also a CDR1 and CDR2 to form a set of CDRs. The CDR or set of CDRs may be a CDR or set of CDRs of any of STIM001, STIM002, STIM002-B, STIM003, STIM004, STIM005, STIM006, STIM007, STIM008 and STIM009, or may be a variant thereof as described herein.

The invention provides antibodies comprising an HCDR1, HCDR2 and/or HCDR3 of any of antibodies STIM001, STIM002, STIM002-B, STIM003, STIM004, STIM005, STIM006, STIM007, STIM008 and STIM009 and/or an LCDR1, LCDR2 and/or LCDR3 of any of these antibodies, e.g. a set of CDRs. The antibody may comprise a set of VH CDRs of one of these antibodies. Optionally it may also comprise a set of VL CDRs of one of these antibodies, and the VL CDRs may be from the same or a different antibody as the VH CDRs.

A VH domain comprising a disclosed set of HCDRs, and/or a VL domain comprising a disclosed set of LCDRs, are also provided by the invention.

Typically, a VH domain is paired with a VL domain to provide an antibody antigen-binding site, although as discussed further below a VH or VL domain alone may be used to bind antigen. The STIM003 VH domain may be paired with the STIM003 VL domain, so that an antibody antigen-binding site is formed comprising both the STIM003 VH and VL domains. Analogous embodiments are provided for the other VH and VL domains disclosed herein. In other embodiments, the STIM003 VH is paired with a VL domain other than the STIM003 VL. Light-chain promiscuity is well established in the art. Again, analogous embodiments are provided by the invention for the other VH and VL domains disclosed herein.

Thus, the VH of any of antibodies STIM001, STIM002, STIM003, STIM004 and STIM005 may be paired with the VL of any of antibodies STIM001, STIM002, STIM003, STIM004 and STIM005. Further, the VH of any of antibodies STIM001, STIM002, STIM002-B, STIM003, STIM004, STIM005, STIM006, STIM007, STIM008 and STIM009 may be paired with the VL of any of antibodies STIM001, STIM002, STIM002-B, STIM003, STIM004, STIM005, STIM006, STIM007, STIM008 or STIM009.

An antibody may comprise one or more CDRs, e.g. a set of CDRs, within an antibody framework. The framework regions may be of human germline gene segment sequences. Thus, the antibody may be a human antibody having a VH domain comprising a set of HCDRs in a human germline framework. Normally the antibody also has a VL domain comprising a set of LCDRs, e.g. in a human germline framework. An antibody “gene segment”, e.g., a VH gene segment, D gene segment, or JH gene segment refers to oligonucleotide having a nucleic acid sequence from which that portion of an antibody is derived, e.g., a VH gene segment is an oligonucleotide comprising a nucleic acid sequence that corresponds to a polypeptide VH domain from FR1 to part of CDR3. Human V, D and J gene segments recombine to generate the VH domain, and human V and J segments recombine to generate the VL domain. The D domain or region refers to the diversity domain or region of an antibody chain. J domain or region refers to the joining domain or region of an antibody chain. Somatic hypermutation may result in an antibody VH or VL domain having framework regions that do not exactly match or align with the corresponding gene segments, but sequence alignment can be used to identify the closest gene segments and thus identify from which particular combination of gene segments a particular VH or VL domain is derived. When aligning antibody sequences with gene segments, the antibody amino acid sequence may be aligned with the amino acid sequence encoded by the gene segment, or the antibody nucleotide sequence may be aligned directly with the nucleotide sequence of the gene segment.

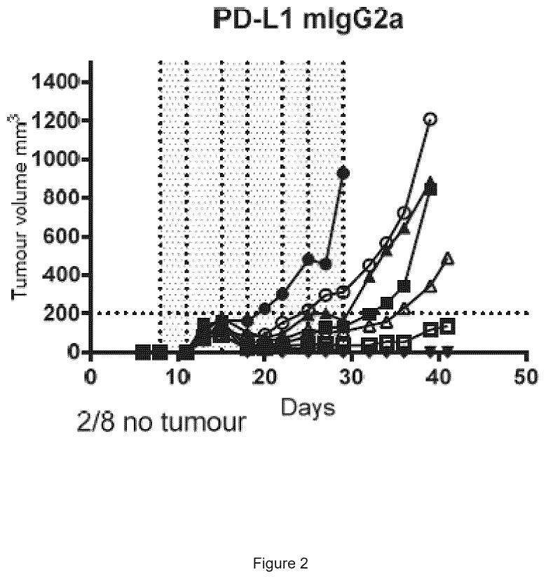

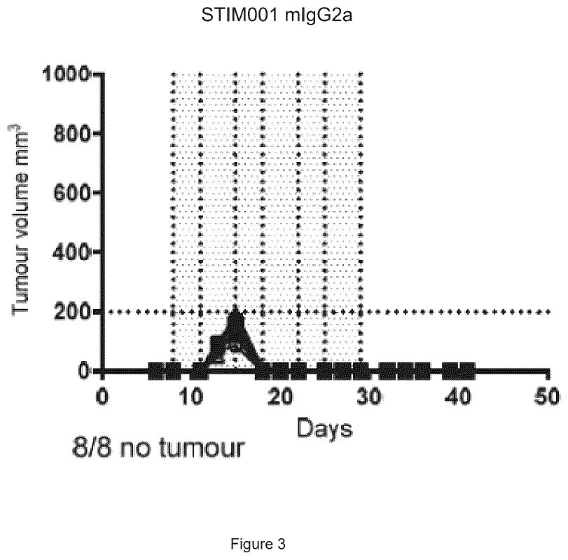

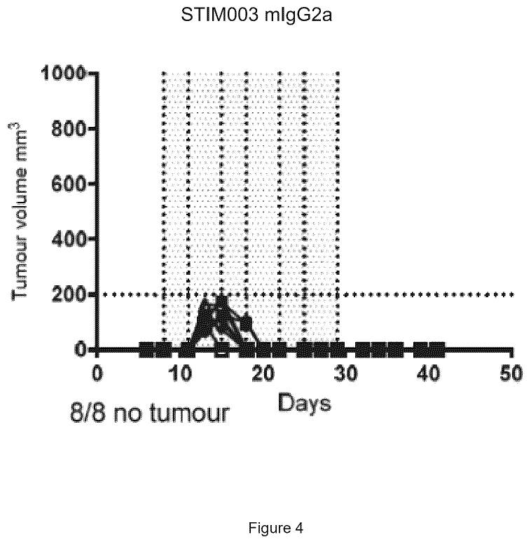

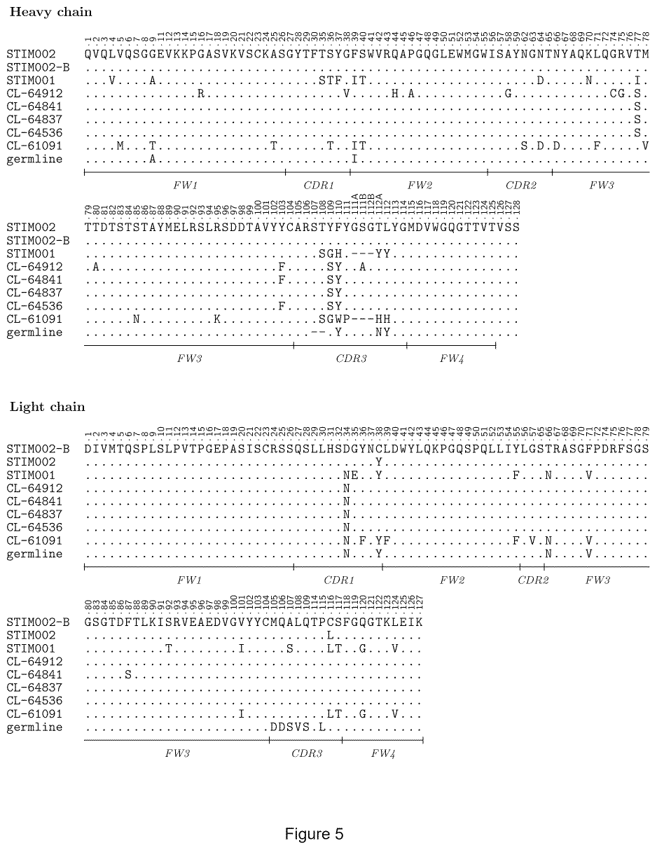

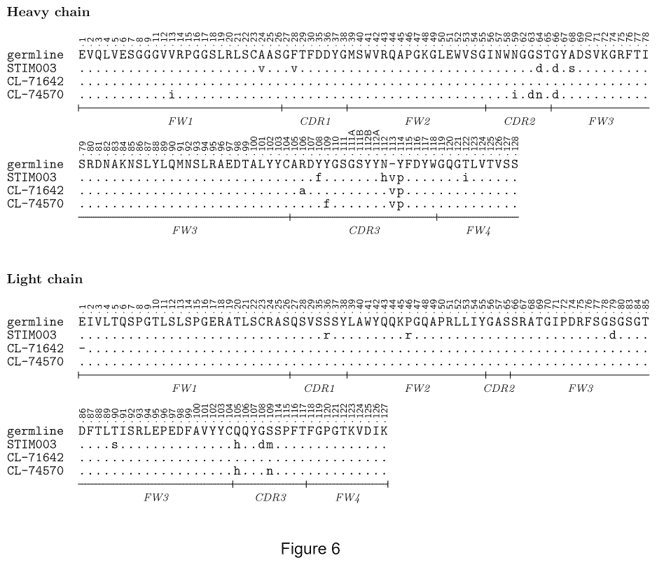

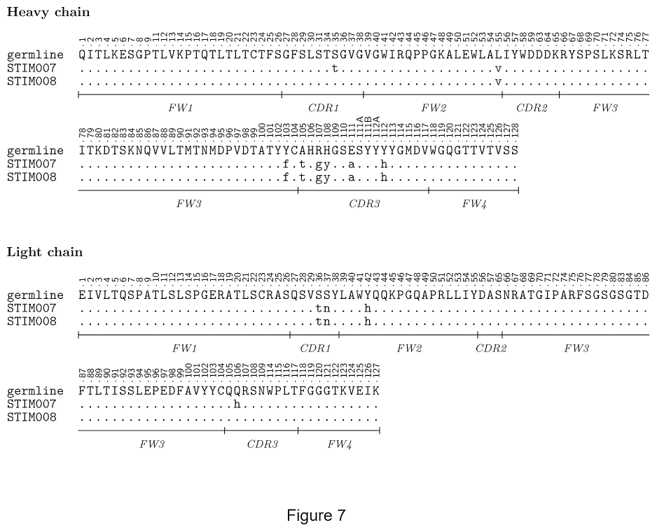

Alignments of STIM antibody VH and VL domain sequences against related antibodies and against human germline sequences are shown in FIG. 5, FIG. 6 and FIG. 7.

An antibody of the invention may be a human antibody or a chimaeric antibody comprising human variable regions and non-human (e.g., mouse) constant regions. The antibody of the invention for example has human variable regions, and optionally also has human constant regions.

Thus, antibodies optionally include constant regions or parts thereof, e.g., human antibody constant regions or parts thereof. For example, a VL domain may be attached at its C-terminal end to antibody light chain kappa or lambda constant domains. Similarly, an antibody VH domain may be attached at its C-terminal end to all or part (e.g. a CH1 domain or Fc region) of an immunoglobulin heavy chain constant region derived from any antibody isotype, e.g. IgG, IgA, IgE and IgM and any of the isotype sub-classes, such as IgG1 or IgG4.

Examples of human heavy chain constant regions are shown in Table S1.

Constant regions of antibodies of the invention may alternatively be non-human constant regions. For example, when antibodies are generated in transgenic animals (examples of which are described elsewhere herein), chimaeric antibodies may be produced comprising human variable regions and non-human (host animal) constant regions. Some transgenic animals generate fully human antibodies. Others have been engineered to generate antibodies comprising chimaeric heavy chains and fully human light chains. Where antibodies comprise one or more non-human constant regions, these may be replaced with human constant regions to provide antibodies more suitable for administration to humans as therapeutic compositions, as their immunogenicity is thereby reduced.

Digestion of antibodies with the enzyme papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognised by Fc receptors (FcR) found on certain types of cells. Digestion of antibodies with the enzyme pepsin, results in the a F(ab′)2 fragment in which the two arms of the antibody molecule remain linked and comprise two-antigen binding sites. The F(ab′)2 fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognise and bind antigen, although at a lower affinity than the entire binding site.

Antibodies disclosed herein may be modified to increase or decrease serum half-life. In one embodiment, one or more of the following mutations: T252L, T254S or T256F are introduced to increase biological half-life of the antibody. Biological half-life can also be increased by altering the heavy chain constant region CH₁ domain or CL region to contain a salvage receptor binding epitope taken from two loops of a CH₂ domain of an Fc region of an IgG, as described in U.S. Pat. Nos. 5,869,046 and 6,121,022, the modifications described therein are incorporated herein by reference. In another embodiment, the Fc hinge region of an antibody or antigen-binding fragment of the invention is mutated to decrease the biological half-life of the antibody or fragment. One or more amino acid mutations are introduced into the CH₂—CH₃ domain interface region of the Fc-hinge fragment such that the antibody or fragment has impaired Staphylococcyl protein A (SpA) binding relative to native Fc-hinge domain SpA binding. Other methods of increasing serum half-life are known to those skilled in the art. Thus, in one embodiment, the antibody or fragment is PEGylated. In another embodiment, the antibody or fragment is fused to an albumin-biding domain, e.g. an albumin binding single domain antibody (dAb). In another embodiment, the antibody or fragment is PASylated (i.e. genetic fusion of polypeptide sequences composed of PAS (XL-Protein GmbH) which forms uncharged random coil structures with large hydrodynamic volume). In another embodiment, the antibody or fragment is XTENylated®/rPEGylated (i.e. genetic fusion of non-exact repeat peptide sequence (Amunix, Versartis) to the therapeutic peptide). In another embodiment, the antibody or fragment is ELPylated (i.e. genetic fusion to ELP repeat sequence (PhaseBio)). These various half-life extending fusions are described in more detail in Strohl, BioDrugs (2015) 29:215-239, which fusions, e.g. in Tables 2 and 6, are incorporated herein by reference.

The antibody may have a modified constant region which increases stability. Thus, in one embodiment, the heavy chain constant region comprises a Ser228Pro mutation. In another embodiment, the antibodies and fragments disclosed herein comprise a heavy chain hinge region that has been modified to alter the number of cysteine residues. This modification can be used to facilitate assembly of the light and heavy chains or to increase or decrease the stability of the antibody.

### 1.6.9. Fc Effector Functions, ADCC, ADCP and CDC

As discussed above, anti-ICOS antibodies can be provided in various isotypes and with different constant regions. Examples of human IgG antibody heavy chain constant region sequences are shown in Table S1. The Fc region of the antibody primarily determines its effector function in terms of Fc binding, antibody-dependent cell-mediated cytotoxicity (ADCC) activity, complement dependent cytotoxicity (CDC) activity and antibody-dependent cell phagocytosis (ADCP) activity. These “cellular effector functions”, as distinct from effector T cell function, involve recruitment of cells bearing Fc receptors to the site of the target cells, resulting in killing of the antibody-bound cell. In addition to ADCC and CDC, the ADCP mechanism [20] represents a means of depleting antibody-bound T cells, and thus targeting high ICOS expressing TRegs for deletion. ²⁰ Gui et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015

Cellular effector functions ADCC, ADCP and/or CDC may also be exhibited by antibodies lacking Fc regions. Antibodies may comprise multiple different antigen-binding sites, one directed to ICOS and another directed to a target molecule where engagement of that target molecule induces ADCC, ADCP and/or CDC, e.g., an antibody comprising two scFv regions joined by a linker, where one scFv can engage an effector cell.

An antibody according to the present invention may be one that exhibits ADCC, ADCP and/or CDC. Alternatively, an antibody according to the present invention may lack ADCC, ADCP and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

A suitable antibody format for some therapeutic applications employs a wild-type human IgG1 constant region. A constant region may be an effector-enabled IgG1 constant region, optionally having ADCC and/or CDC and/or ADCP activity. A suitable wild type human IgG1 constant region sequence is SEQ ID NO: 340 (IGHG1*01). Further examples of human IgG1 constant regions are shown in Table S1.

For testing of candidate therapeutic antibodies in mouse models of human disease, an effector positive mouse constant region, such as mouse IgG2a (mIgG2a), may be included instead of an effector positive human constant region.

A constant region may be engineered for enhanced ADCC and/or CDC and/or ADCP.

The potency of Fc-mediated effects may be enhanced by engineering the Fc domain by various established techniques. Such methods increase the affinity for certain Fc-receptors, thus creating potential diverse profiles of activation enhancement. This can achieved by modification of one or several amino acid residues [21]. Human IgG1 constant regions containing specific mutations or altered glycosylation on residue Asn297 (e.g., N297Q, EU index numbering) have been shown to enhance binding to Fc receptors. Example mutations are one or more of the residues selected from 239, 332 and 330 for human IgG1 constant regions (or the equivalent positions in other IgG isotypes). An antibody may thus comprise a human IgG1 constant region having one or more mutations independently selected from N297Q, S239D, I332E and A330L (EU index numbering). A triple mutation (M252Y/S254T/T256E) may be used to enhance binding to FcRn, and other mutations affecting FcRn binding are discussed in Table 2 of [22], any of which may be employed in the present invention. ²¹ Lazar et al., 2006, Proc. Natl. Acad. Sci. U.S.A., March 14; 103(11):4005-10²² Dall et al., Immunol 2002; 169:5171-5180

Increased affinity for Fc receptors can also be achieved by altering the natural glycosylation profile of the Fc domain by, for example, generating under fucosylated or de-fucosylated variants [23]. Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. For example, to increase ADCC, residues in the hinge region can be altered to increase binding to Fc-gamma RIII [24]. Thus, an antibody may comprise a human IgG heavy chain constant region that is a variant of a wild-type human IgG heavy chain constant region, wherein the variant human IgG heavy chain constant region binds to human Fcγ receptors selected from the group consisting of FcγRIIB and FcγRIIA with higher affinity than the wild type human IgG heavy chain constant region binds to the human Fcγ receptors. The antibody may comprise a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. The variant human IgG heavy chain constant region can be a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). The enhancement of CDC may be achieved by amino acid changes that increase affinity for C1q, the first component of the classic complement activation cascade [25]. Another approach is to create a chimeric Fc domain created from human IgG1 and human IgG3 segments that exploit the higher affinity of IgG3 for C1q [26]. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system). ²³ Natsume et al., 2009, Drug Des. Devel. Ther., 3:7-16 or by Zhou Q., Biotechnol. Bioeng., 2008 Feb. 15, 99(3):652-65)²⁴ Shields et al., 2001, J. Biol. Chem., March 2; 276(9):6591-604)²⁵ Idusogie et al., J. Immunol., 2001, 166:2571-2575²⁶ Natsume et al., 2008, Cancer Res., 68: 3863-3872

WO2008/137915 described anti-ICOS antibodies with modified Fc regions having enhanced effector function. The antibodies were reported to mediate enhanced ADCC activity as compared to the level of ADCC activity mediated by a parent antibody comprising the VH and VK domains and a wild type Fc region. Antibodies according to the present invention may employ such variant Fc regions having effector function as described therein.

ADCC activity of an antibody may be determined in an assay, such as the assays disclosed in WO2008/137915. ADCC activity of an anti-ICOS antibody may be determined in vitro using an ICOS positive T cell line as described in Example 10 of U.S. Pat. No. 9,957,323. ADCC activity of an anti-PD-L1 antibody may be determined in vitro in an ADCC assay using PD-L1 expressing cells.

For certain applications (such as in the context of vaccination) it may be preferred to use antibodies without Fc effector function. Antibodies may be provided without a constant region, or without an Fc region—examples of such antibody formats are described elsewhere herein. Alternatively, an antibody may have a constant region which is effector null. An antibody may have a heavy chain constant region that does not bind Fcγ receptors, for example the constant region may comprise a Leu235Glu mutation (i.e., where the wild type leucine residue is mutated to a glutamic acid residue). Another optional mutation for a heavy chain constant region is Ser228Pro, which increases stability. A heavy chain constant region may be an IgG4 comprising both the Leu235Glu mutation and the Ser228Pro mutation. This “IgG4-PE” heavy chain constant region is effector null.

An alternative effector null human constant region is a disabled IgG1. A disabled IgG1 heavy chain constant region may contain alanine at position 235 and/or 237 (EU index numbering), e.g., it may be a IgG1*01 sequence comprising the L235A and/or G237A mutations (“LAGA”).

A variant human IgG heavy chain constant region may comprise one or more amino acid mutations that reduce the affinity of the IgG for human FcγRIIIA, human FcγRIIA, or human FcγRI. In one embodiment, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V305I, A330L, I332E, E333A, K334A, A339T, and P396L (EU index numbering system). In one embodiment, the variant human IgG heavy chain constant region comprises a set of amino acid mutations selected from the group consisting of: S239D; T256A; K290A; S298A; I332E; E333A; K334A; A339T; S239D and I332E; S239D, A330L, and I332E; S298A, E333A, and K334A; G236A, S239D, and I332E; and F243L, R292P, Y300L, V305I, and P396L (EU index numbering system). In one embodiment, the variant human IgG heavy chain constant region comprises a S239D, A330L, or I332E amino acid mutations (EU index numbering system). In one embodiment, the variant human IgG heavy chain constant region comprises an S239D and I332E amino acid mutations (EU index numbering system). In one embodiment, the variant human IgG heavy chain constant region is a variant human IgG1 heavy chain constant region comprising the S239D and I332E amino acid mutations (EU index numbering system). In one embodiment, the antibody or fragment comprises an afucosylated Fc region. In another embodiment, the antibody or fragment thereof is defucosylated. In another embodiment, the antibody or fragment is under fucosylated.

An antibody may have a heavy chain constant region that binds one or more types of Fc receptor but does not induce cellular effector functions, i.e., does not mediate ADCC, CDC or ADCP activity. Such a constant region may be unable to bind the particular Fc receptor(s) responsible for triggering ADCC, CDC or ADCP activity.

### 1.6.10. Generating and Modifying Antibodies

Methods for identifying and preparing antibodies are well known. Antibodies may be generated using transgenic mice (eg, the Kymouse™, Velocimouse®, Omnimouse®, Xenomouse®, HuMab Mouse® or MeMo Mouse®), rats (e.g., the Omnirat®), camelids, sharks, rabbits, chickens or other non-human animals immunised with ICOS or a fragment thereof or a synthetic peptide comprising an ICOS sequence motif of interest, followed optionally by humanisation of the constant regions and/or variable regions to produce human or humanised antibodies. In an example, display technologies can be used, such as yeast, phage or ribosome display, as will be apparent to the skilled person. Standard affinity maturation, e.g., using a display technology, can be performed in a further step after isolation of an antibody lead from a transgenic animal, phage display library or other library. Representative examples of suitable technologies are described in US20120093818 (Amgen, Inc), which is incorporated by reference herein in its entirety, eg, the methods set out in paragraphs [0309] to [0346].

Immunisation of an ICOS knock out non-human animal with human ICOS antigen facilitates the generation of antibodies that recognise both human and non-human ICOS. As described herein and illustrated in the Examples, an ICOS knock out mouse can be immunised with cells expressing human ICOS to stimulate production of antibodies to human and mouse ICOS in the mouse, which can be recovered and tested for binding to human ICOS and to mouse ICOS. Cross-reactive antibodies can thus be selected, which may be screened for other desirable properties as described herein. Methods of generating antibodies to an antigen (e.g., a human antigen), through immunisation of animals with the antigen where expression of the endogenous antigen (e.g, endogenous mouse antigen) has been knocked-out in the animal, may be performed in animals capable of generating antibodies comprising human variable domains. The genomes of such animals can be engineered to comprise a human or humanised immunoglobulin locus encoding human variable region gene segments, and optionally an endogenous constant region or a human constant region. Recombination of the human variable region gene segments generates human antibodies, which may have either a non-human or human constant region. Non-human constant regions may subsequently be replaced by human constant regions where the antibody is intended for in vivo use in humans. Such methods and knock-out transgenic animals are described in WO2013/061078.

Generally, a Kymouse™, VELOCIMMUNE® or other mouse or rat (optionally an ICOS knock out mouse or rat, as noted) can be challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimaeric antibodies or the variable domains of the light and heavy chains may be isolated directly from antigen-specific lymphocytes.

Initially, high affinity chimaeric antibodies are isolated having a human variable region and a mouse constant region. The antibodies are characterised and selected for desirable characteristics, including affinity, selectivity, agonism, T-cell dependent killing, neutralising potency, epitope, etc. The mouse constant regions are optionally replaced with a desired human constant region to generate the fully human antibody of the invention, for example wild-type or modified IgG1 or IgG4 (for example, SEQ ID NO: 751, 752, 753 in US2011/0065902 (which is incorporated by reference herein in its entirety). While the constant region selected may vary according to specific use, high affinity antigen-binding and target specificity characteristics reside in the variable region.

Thus, in a further aspect, the present invention provides a transgenic non-human mammal having a genome comprising a human or humanised immunoglobulin locus, wherein the mammal does not express ICOS. The mammal may for instance be a knock-out mouse or rat, or other laboratory animal species. Transgenic mice such as the Kymouse™ contain human heavy and light chain immunoglobulin loci inserted at the corresponding endogenous mouse immunoglobulin loci. A transgenic mammal according to the present invention may be one that contains such targeted insertions, or it may contain human heavy and light chain immunoglobulin loci or immunoglobulin genes that are randomly inserted in its genome, inserted at a locus other than the endogenous Ig locus, or provided on an additional chromosome or chromosomal fragment.

Further aspects of the invention are the use of such non-human mammals for producing antibodies to ICOS, and methods of producing antibodies or antibody heavy and/or light chain variable domains in such mammals.

A method of producing an antibody that binds the extracellular domain of human and non-human ICOS may comprise providing a transgenic non-human mammal having a genome comprising a human or humanised immunoglobulin locus, wherein the mammal does not express ICOS, and
(a) immunising the mammal with human ICOS antigen (e.g., with cells expressing human ICOS or with purified recombinant ICOS protein);
(b) isolating antibodies generated by the mammal;
(c) testing the antibodies for ability to bind human ICOS and non-human ICOS; and
(d) selecting one or more antibodies that binds both human and non-human ICOS.

Testing for ability to bind human ICOS and non-human ICOS may be done using surface plasmon resonance, HTRF, FACS or any other method described herein. Optionally, binding affinities for human and mouse ICOS are determined. The affinity, or fold-difference in affinity, of binding to human ICOS and mouse ICOS may be determined, and antibodies displaying species cross-reactivity may thus be selected (affinity thresholds and fold-differences that may be used as selection criteria are exemplified elsewhere herein). Neutralising potency, or fold difference in neutralising potency, of the antibody for inhibiting human and mouse ICOS ligand binding to the human and mouse ICOS receptor respectively may also or alternatively be determined as a way to screen for cross-reactive antibodies, e.g., in an HTRF assay. Again, possible thresholds and fold-differences that may be used as selection criteria are exemplified elsewhere herein.

The method may comprise testing the antibodies for ability to bind non-human ICOS from the same species or from a different species as the immunised mammal. Thus, where the transgenic mammal is a mouse (e.g., a Kymouse™), antibodies may be tested for ability to bind mouse ICOS. Where the transgenic mammal is a rat, antibodies may be tested for ability to bind rat ICOS. However, it may be equally useful to determine cross-reactivity of an isolated antibody for non-human ICOS of another species. Thus, antibodies generated in goats may be tested for binding to rat or mouse ICOS. Optionally, binding to goat ICOS may be determined instead or additionally.

In other embodiments, the transgenic non-human mammal may be immunised with non-human ICOS, optionally ICOS of the same mammalian species (e.g., an ICOS knock-out mouse may be immunised with mouse ICOS) instead of human ICOS. Affinity of isolated antibodies for binding to human ICOS and non-human ICOS is then determined in the same way, and antibodies that bind both human and non-human ICOS are selected.

Nucleic acid encoding an antibody heavy chain variable domain and/or an antibody light chain variable domain of a selected antibody may be isolated. Such nucleic acid may encode the full antibody heavy chain and/or light chain, or the variable domain(s) without associated constant region(s). As noted, encoding nucleotide sequences may be obtained directly from antibody-producing cells of a mouse, or B cells may be immortalised or fused to generate hybridomas expressing the antibody, and encoding nucleic acid obtained from such cells. Optionally, nucleic acid encoding the variable domain(s) is then conjugated to a nucleotide sequence encoding a human heavy chain constant region and/or human light chain constant region, to provide nucleic acid encoding a human antibody heavy chain and/or human antibody light chain, e.g., encoding an antibody comprising both the heavy and light chain. As described elsewhere herein, this step is particularly useful where the immunised mammal produces chimaeric antibodies with non-human constant regions, which are preferably replaced with human constant regions to generate an antibody that will be less immunogenic when administered to humans as a medicament. Provision of particular human isotype constant regions is also significant for determining the effector function of the antibody, and a number of suitable heavy chain constant regions are discussed herein.

Other alterations to nucleic acid encoding the antibody heavy and/or light chain variable domain may be performed, such as mutation of residues and generation of variants, as described herein.

The isolated (optionally mutated) nucleic acid may be introduced into host cells, e.g., CHO cells as discussed. Host cells are then cultured under conditions for expression of the antibody, or of the antibody heavy and/or light chain variable domain, in any desired antibody format. Some possible antibody formats are described herein, e.g., whole immunoglobulins, antigen-binding fragments, and other designs.

Variable domain amino acid sequence variants of any of the VH and VL domains or CDRs whose sequences are specifically disclosed herein may be employed in accordance with the present invention, as discussed.

There are many reasons why it may be desirable to create variants, which include improving the antibody sequence for large-scale manufacturing, facilitating purification, enhancing stability or improving suitability for inclusion in a desired pharmaceutical formulation. Protein engineering work can be performed at one or more target residues in the antibody sequence, e.g., to substituting one amino acid with an alternative amino acid (optionally, generating variants containing all naturally occurring amino acids at this position, with the possible exception of Cys and Met), and monitoring the impact on function and expression to determine the best substitution. It is in some instances undesirable to substitute a residue with Cys or Met, or to introduce these residues into a sequence, as to do so may generate difficulties in manufacturing—for instance through the formation of new intramolecular or intermolecular cysteine-cysteine bonds. Where a lead candidate has been selected and is being altered for manufacturing and clinical development, it will generally be desirable to change its antigen-binding properties as little as possible, or at least to retain the affinity and potency of the parent molecule. However, variants may also be generated in order to modulate key antibody characteristics such as affinity, cross-reactivity or neutralising potency.

An antibody may comprise a set of H and/or L CDRs of any of the disclosed antibodies with one or more amino acid mutations within the disclosed set of H and/or L CDRs. The mutation may be an amino acid substitution, deletion or insertion. Thus for example there may be one or more amino acid substitutions within the disclosed set of H and/or L CDRs. For example, there may be up to 12, 11, 10, 9, 8, 7, 6, 5, 4, 3 or 2 mutations e.g. substitutions, within the set of H and/or L CDRs. For example, there may be up to 6, 5, 4, 3 or 2 mutations, e.g. substitutions, in HCDR3 and/or there may be up to 6, 5, 4, 3, or 2 mutations, e.g. substitutions, in LCDR3. An antibody may comprise the set of HCDRs, LCDRs or a set of 6 (H and L) CDRs shown for any STIM antibody herein or may comprise that set of CDRs with one or two conservative substitutions.

One or more amino acid mutations may optionally be made in framework regions of an antibody VH or VL domain disclosed herein. For example, one or more residues that differ from the corresponding human germline segment sequence may be reverted to germline. Human germline gene segment sequences corresponding to VH and VL domains of example anti-ICOS antibodies are indicated in Table E12-1, Table E12-2 and Table E12-3, and alignments of antibody VH and VL domains to corresponding germline sequences are shown in the drawings.

An antibody may comprise a VH domain that has at least 60, 70, 80, 85, 90, 95, 98 or 99% amino acid sequence identity with a VH domain of any of the antibodies shown in the appended sequence listing, and/or comprising a VL domain that has at least 60, 70, 80, 85, 90, 95, 98 or 99% amino acid sequence identity with a VL domain of any of those antibodies. Algorithms that can be used to calculate % identity of two amino acid sequences include e.g. BLAST, FASTA, or the Smith-Waterman algorithm, e.g. employing default parameters. Particular variants may include one or more amino acid sequence alterations (addition, deletion, substitution and/or insertion of an amino acid residue).

Alterations may be made in one or more framework regions and/or one or more CDRs. Variants are optionally provided by CDR mutagenesis. The alterations normally do not result in loss of function, so an antibody comprising a thus-altered amino acid sequence may retain an ability to bind ICOS. It may retain the same quantitative binding ability as an antibody in which the alteration is not made, e.g. as measured in an assay described herein. The antibody comprising a thus-altered amino acid sequence may have an improved ability to bind ICOS.

Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the invention are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring.

The term “variant” as used herein refers to a peptide or nucleic acid that differs from a parent polypeptide or nucleic acid by one or more amino acid or nucleic acid deletions, substitutions or additions, yet retains one or more specific functions or biological activities of the parent molecule. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide).

In some aspects, one can use “synthetic variants”, “recombinant variants”, or “chemically modified” polynucleotide variants or polypeptide variants isolated or generated using methods well known in the art. “Modified variants” can include conservative or non-conservative amino acid changes, as described below. Polynucleotide changes can result in amino acid substitutions, additions, deletions, fusions and truncations in the polypeptide encoded by the reference sequence. Some aspects use include insertion variants, deletion variants or substituted variants with substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties (e.g., acidic, basic, positively or negatively charged, polar or nonpolar, etc.). Conservative amino acid substitutions include replacement of a leucine with an isoleucine or valine, an aspartate with a glutamate, or a threonine with a serine. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

One can select the amino acid that will substitute an existing amino acid based on the location of the existing amino acid, including its exposure to solvents (i.e., if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. MoI Biol, 1999, 217, 721-739 and Taylor et al, J. Theor. Biol. 119(1986); 205-218 and S. French and B. Robson, J. Mol. Evol. 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

The invention includes methods of producing antibodies containing VH and/or VL domain variants of the antibody VH and/or VL domains shown in the appended sequence listing. Such antibodies may be produced by a method comprising
(i) providing, by way of addition, deletion, substitution or insertion of one or more amino acids in the amino acid sequence of a parent antibody VH domain, an antibody VH domain that is an amino acid sequence variant of the parent antibody VH domain,
   wherein the parent antibody VH domain is the VH domain of any of antibodies STIM001, STIM002, STIM002-B, STIM003, STIM004, STIM005, STIM006, STIM007, STIM008 and STIM009 or a VH domain comprising the heavy chain complementarity determining regions of any of those antibodies,
(ii) optionally combining the VH domain thus provided with a VL domain, to provide a VH/VL combination, and
(iii) testing the VH domain or VH/VL domain combination thus provided to identify an antibody with one or more desired characteristics.

Desired characteristics include binding to human ICOS, binding to mouse ICOS, and binding to other non-human ICOS such as cynomolgus ICOS. Antibodies with comparable or higher affinity for human and/or mouse ICOS may be identified. Other desired characteristics include increasing effector T cell function indirectly, via depletion of immunosuppressive TRegs, or directly, via ICOS signalling activation on T effector cells. Identifying an antibody with a desired characteristic may comprise identifying an antibody with a functional attribute described herein, such as its affinity, cross-reactivity, specificity, ICOS receptor agonism, neutralising potency and/or promotion of T cell dependent killing, any of which may be determined in assays as described herein.

When VL domains are included in the method, the VL domain may be a VL domain of any of STIM001, STIM002, STIM002-B, STIM003, STIM004, STIM005, STIM006, STIM007, STIM008 or STIM009, or may be a variant provided by way of addition, deletion, substitution or insertion of one or more amino acids in the amino acid sequence of a parent VL domain, wherein the parent VL domain is the VL domain of any of STIM001, STIM002, STIM002-B, STIM003, STIM004, STIM005, STIM006, STIM007, STIM008 and STIM009 or a VL domain comprising the light chain complementarity determining regions of any of those antibodies.

Methods of generating variant antibodies may optionally comprise producing copies of the antibody or VH/VL domain combination. Methods may further comprise expressing the resultant antibody. It is possible to produce nucleotide sequences corresponding to a desired antibody VH and/or VL domain, optionally in one or more expression vectors. Suitable methods of expression, including recombinant expression in host cells, are set out in detail herein.

### 1.6.11. Encoding Nucleic Acids and Methods of Expression

Isolated nucleic acid may be provided, encoding antibodies according to the present invention. Nucleic acid may be DNA and/or RNA. Genomic DNA, cDNA, mRNA or other RNA, of synthetic origin, or any combination thereof can encode an antibody.

The present invention provides constructs in the form of plasmids, vectors, transcription or expression cassettes which comprise at least one polynucleotide as above. Exemplary nucleotide sequences are included in the sequence listing. Reference to a nucleotide sequence as set out herein encompasses a DNA molecule with the specified sequence, and encompasses a RNA molecule with the specified sequence in which U is substituted for T, unless context requires otherwise.

The present invention also provides a recombinant host cell that comprises one or more nucleic acids encoding the antibody. Methods of producing the encoded antibody may comprise expression from the nucleic acid, e.g., by culturing recombinant host cells containing the nucleic acid. The antibody may thus be obtained, and may be isolated and/or purified using any suitable technique, then used as appropriate. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals.

The expression of antibodies and antibody fragments in prokaryotic cells is well established in the art. A common bacterial host is E. coli. Expression in eukaryotic cells in culture is also available to those skilled in the art as an option for production. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells, human embryonic retina cells and many others.

Vectors may contain appropriate regulatory sequences, including promoter sequences, terminator sequences, polyadenylation sequences, enhancer sequences, marker genes and other sequences as appropriate. Nucleic acid encoding an antibody can be introduced into a host cell. Nucleic acid can be introduced to eukaryotic cells by various methods, including calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques include calcium chloride transformation, electroporation and transfection using bacteriophage. The introduction may be followed by expressing the nucleic acid, e.g., by culturing host cells under conditions for expression of the gene, then optionally isolating or purifying the antibody.

Nucleic acid of the invention may be integrated into the genome (e.g. chromosome) of the host cell. Integration may be promoted by inclusion of sequences that promote recombination with the genome, in accordance with standard techniques.

The present invention also provides a method that comprises using nucleic acid described herein in an expression system in order to express an antibody.

### 1.6.12. Therapeutic Use

An antibody (e.g., a full length antibody or an antigen-binding fragment thereof) described herein may be used in a method of treatment of the human or animal body by therapy. The antibodies find use in increasing effector T cell response, which is of benefit for a range of diseases or conditions, including treating cancers or solid tumours and in the context of vaccination. Increased Teff response may be achieved using an antibody that modulates the balance or ratio between Teffs and Tregs in favour of Teff activity.

Anti-ICOS antibodies may be used for depleting regulatory T cells and/or increasing effector T cell response in a patient, and may be administered to a patient to treat a disease or condition amenable to therapy by depleting regulatory T cells and/or increasing effector T cell response.

An antibody of the present invention, or a composition comprising such an antibody molecule or its encoding nucleic acid, may be used or provided for use in any such method. Use of the antibody, or of a composition comprising it or its encoding nucleic acid, for the manufacture of a medicament for use in any such method is also envisaged. The method typically comprises administering the antibody or composition to a mammal. Suitable formulations and methods of administration are described elsewhere herein.

One envisaged therapeutic use of the antibodies is treatment of cancer. The cancer may be a solid tumour, e.g., renal cell cancer (optionally renal cell carcinoma, e.g., clear cell renal cell carcinoma), head and neck cancer, melanoma (optionally malignant melanoma), non-small cell lung cancer (e.g., adenocarcinoma), bladder cancer, ovarian cancer, cervical cancer, gastric cancer, liver cancer, pancreatic cancer, breast cancer, testicular germ cell carcinoma, or the metastases of a solid tumour such as those listed, or it may be a liquid haematological tumour e.g., lymphoma (such as Hodgkin's lymphoma or Non-Hodgkin's lymphoma, e.g., diffuse large B-cell lymphoma, DLBCL) or leukaemia (e.g., acute myeloid leukaemia). An anti-ICOS antibody may enhance tumour clearance in melanoma, head and neck cancer and non-small cell lung cancer and other cancers with a moderate to high mutational load [27]. In some embodiments, the cancer is breast cancer. In some embodiments, the cancer is triple negative breast cancer. In some embodiments, the cancer is head and neck squamous cell carcinoma. In some embodiments, the cancer is penile cancer. In some embodiments, the cancer is pancreatic cancer. In some embodiments, the cancer is non-small cell lung cancer. In some embodiments, the cancer is hepatocellular carcinoma. In some embodiments, the cancer is esophageal cancer. In some embodiments, the cancer is gastric cancer. In some embodiments, the cancer is melanoma. In some embodiments, the cancer is renal cell carcinoma. In some embodiments, the cancer is cervical cancer. In some embodiments, the cancer is an advanced cancer. In some embodiments, the cancer is a metastatic cancer. ²⁷ Alexandrov L B, et al. Signatures of mutational processes in human cancer. Nature. 2013 Aug. 22; 500(7463):415-21

In some embodiments, treatment with one or more doses of an anti-ICOS antibody or antigen-binding fragment thereof results in a partial anti-tumour response. In some embodiments, treatment with one or more doses of an anti-ICOS antibody or antigen-binding fragment thereof results in a complete anti-tumour response. In some embodiments, treatment with one or more doses of an anti-ICOS antibody or antigen-binding fragment thereof and one or more doses of an anti-PD-L1 antibody or antigen-binding fragment thereof results in a partial anti-tumour response. In some embodiments, treatment with one or more doses of an anti-ICOS antibody or antigen-binding fragment thereof and one or more doses of an anti-PD-L1 antibody or antigen-binding fragment thereof results in a complete anti-tumour response.

In some embodiments, the anti-ICOS antibody or antigen-binding fragment thereof comprises the CDR sequences of KY1044. In some embodiments, the anti-ICOS antibody or antigen-binding fragment thereof comprises the CDR sequences of KY1044. In some embodiments, the anti-ICOS antibody or antigen-binding fragment thereof comprises the heavy and light chain variable domain sequences of KY1044. In some embodiments, the anti-ICOS antibody or antigen-binding fragment thereof comprises the heavy and light chain sequences of KY1044. In some embodiments, the anti-ICOS antibody is KY1044. In some embodiments, the anti-PD-L1 antibody is atezolizumab. In some embodiments, treatment with KY1044 promotes the efficacy of the anti-PD-L1 antibody (e.g., atezolizumab).

By enhancing patients' immune response to their neoplastic lesions, immunotherapy using an anti-ICOS antibody offers the prospect of durable cures or long-term remissions, potentially even in the context of late stage disease.

Cancers are a diverse group of diseases, but anti-ICOS antibodies offer the possibility of treating a range of different cancers by exploiting the patient's own immune system, which has the potential to kill any cancer cell through recognition of mutant or overexpressed epitopes that distinguish cancer cells from normal tissue. By modulating the Teff/Treg balance, anti-ICOS antibodies can enable and/or promote immune recognition and killing of cancer cells. While anti-ICOS antibodies are therefore useful therapeutic agents for a wide variety of cancers, there are particular categories of cancers for which anti-ICOS therapy is especially suited and/or where anti-ICOS therapy can be effective when other therapeutic agents are not.

One such group is cancer that is positive for expression of ICOS ligand. Cancer cells may acquire expression of ICOS ligand, as has been described for melanoma [28]. Expression of ICOS ligand may provide the cells with a selective advantage as the surface-expressed ligand binds ICOS on Tregs, promoting the expansion and activation of the Tregs and thereby suppressing the immune response against the cancer. Cancer cells expressing ICOS ligand may depend for their survival on this suppression of the immune system by Tregs, and would thus be vulnerable to treatment with anti-ICOS antibodies that target the Tregs. This applies also to cancers derived from cells that naturally express ICOS ligand. Continued expression of ICOS ligand by these cells again provides a survival advantage through immune suppression. A cancer expressing ICOS ligand may be derived from antigen-presenting cells such as B cells, dendritic cells and monocytes and may be a liquid haematological tumour such as those mentioned herein. Interestingly it has been shown that these types of cancer are also high in ICOS and FOXP3 expression (TCGA data)—see Example 6. Example 1 herein demonstrates efficacy of exemplary anti-ICOS antibodies in treating tumours derived from cancerous B cells (A20 syngeneic cells) that express ICOS ligand. ²⁸ Martin-Orozco et al., Melanoma Cells Express ICOS Ligand to Promote the Activation and Expansion of T-Regulatory Cells, Cancer Research 70(23):9581-9590 2010

Accordingly, anti-ICOS antibodies can be used in methods of treating cancers that are positive for expression of ICOS ligand. Further, a cancer to be treated with anti-ICOS antibody according to the present invention may be one that is positive for expression of ICOS and/or FOXP3, and optionally also expresses ICOS ligand.

Patients may undergo testing to determine whether their cancer is positive for expression of the protein of interest (e.g., ICOS ligand, ICOS and/or FOXP3), for example by taking a test sample (e.g., tumour biopsy) from the patient and determining expression of the protein of interest. Patients whose cancer has been characterised as positive for expression of one, two or all such proteins of interest are selected for treatment with anti-ICOS antibody. As discussed elsewhere herein, anti-ICOS antibody may be used as a monotherapy or in combination with one or more other therapeutic agents.

Anti-ICOS antibodies also offer hope to patients whose cancers are refractory to treatment with antibodies or other drugs directed to immune checkpoint molecules such as CTLA-4, PD-1, PD-L1, CD137, GITR or CD73. These immunotherapies are effective against some cancers but in some cases a cancer may not respond, or it may become unresponsive to continued treatment with the antibody. In common with antibodies to immune checkpoint inhibitors, anti-ICOS antibodies modulate the patient's immune system—nevertheless an anti-ICOS antibody may succeed where such other antibodies fail. It is shown herein that animals carrying A20 B cell lymphomas could be treated with anti-ICOS antibodies to reduce growth of the tumour, shrink the tumour and indeed clear the tumour from the body, whereas treatment with an anti-PD-L1 antibody was no better than control. The A20 cell line has also been reported to be resistant to anti-CTLA-4 [29]. ²⁹ Houot et al., Therapeutic effect of CD137 immunomodulation in lymphoma and its enhancement by Treg depletion, Blood 114:3431-3438 2009

Accordingly, anti-ICOS antibodies can be used in methods of treating cancers that are refractory to treatment with one or more immunotherapies, such as (any or all of) an anti-CTLA-4 antibody, anti-PD1 antibody, anti-PD-L1 antibody, anti-CD137 antibody, anti-GITR antibody, or anti-CD73 antibody. A cancer may be characterised as being refractory to treatment with an antibody or other drug if treatment with that antibody or drug does not significantly reduce growth of the cancer, e.g., if a tumour continues to grow or does not reduce in size or if after a response period the tumour re-initiates its growth. Non-response to a therapeutic agent may be determined ex vivo by testing a sample (e.g., tumour biopsy sample) for cancer cell killing or growth inhibition, and/or in the clinical setting by observing (e.g., using an imaging technology, including MRI) that a patient treated with the therapy is not responding to treatment. Patients whose cancer has been characterised as refractory to treatment with such an immunotherapy are selected for treatment with anti-ICOS antibody.

Further, anti-ICOS antibodies may be used to treat B-cell derived cancer that is resistant to treatment with an anti-CD20 antibody. Anti-ICOS antibodies represent a treatment for cancers that fail to respond to, or become resistant to, therapy with anti-CD20 antibodies like rituximab. Anti-ICOS antibody may be used as a second-line (or further, or additional) treatment for such cancers. The anti-CD20 antibody resistant cancer may be a B cell cancer, e.g., B cell lymphoma, such as diffuse large B cell lymphoma. Resistance of a cancer to anti-CD20 may be determined ex vivo by testing a sample (e.g., tumour biopsy sample) for cancer cell killing or growth inhibition by anti-CD20 antibody, and/or in the clinical setting by observing that a patient treated with the anti-CD20 antibody is not responding to treatment. Alternatively, or additionally, the cancer (e.g., a tumour biopsy sample) may be tested to assess expression of CD20, where an absence or low level of CD20 expression indicates loss of sensitivity to anti-CD20 antibody.

Samples obtained from patients may thus be tested to determine surface expression of a protein of interest, for example ICOS ligand, ICOS, FOXP3 and/or a target receptor to which another therapeutic agent (e.g., anti-receptor antibody) is directed. The target receptor may be CD20 (to which anti-CD20 antibody therapy such as rituximab is directed), or another receptor such as PD1, EGFR, HER2 or HER3. Surface expression of ICOS ligand, ICOS, FOXP3 and/or lack or loss of surface expression of the target receptor is an indication that the cancer is susceptible to anti-ICOS antibody therapy. Anti-ICOS antibodies can be provided for administration to a patient whose cancer is characterised by surface expression of ICOS ligand, ICOS, FOXP3 and/or lack or loss of surface expression of a target receptor, optionally where the patient has been previously treated with anti-CTLA4, anti-PD1, anti-PD-L1 or with an antibody to the target receptor and has not responded or has stopped responding to treatment with that antibody, as measured for example by continued or renewed cancer cell growth, e.g., increase in tumour size.

Any suitable method may be employed to determine whether cancer cells test positive for surface expression of a protein such as ICOS ligand, CD20 or other target receptors mentioned herein. A typical method is immunohistochemistry, where a sample of the cells (e.g., a tumour biopsy sample) is contacted with an antibody for the protein of interest, and binding of antibody is detected using a labelled reagent—typically a second antibody that recognises the Fc region of the first antibody and carries a detectable label such as a fluorescent marker. A sample may be declared to test positive where at least 5% of cells are labelled, as visualised by cell staining or other detection of the label. Optionally a higher cut-off such as 10% or 25% may be used. The antibody will generally be used in excess. Reagent antibodies to the molecules of interest are available or may be generated by straightforward methods. To test for ICOS ligand, the antibody MAB1651 is currently available from R&D systems as a mouse IgG that recognises human ICOS ligand. To test for CD20 expression, rituximab may be used. Detection of mRNA levels of the ICOS ligand or target receptor of interest is an alternative technique [27].

A further indication that a tumour will respond to treatment with anti-ICOS antibody is the presence of Tregs in the tumour microenvironment. Activated Tregs are characterised by ICOS-high and Foxp3-high surface expression. The presence of Tregs in a tumour, especially in elevated numbers, provides a further basis on which a patient may be selected for treatment with anti-ICOS antibody. Tregs may be detected in a tumour biopsy sample ex vivo, for example by immunohistochemistry (assaying for co-expression of both Foxp3 and ICOS, using antibodies to the target protein followed by detection of labels, as described above) or by single cell dispersion of the sample for use in FACS with labelled antibodies to ICOS and Foxp3.

The anti-ICOS antibodies may be used for treating cancers associated with infectious agents, such as virally-induced cancers. In this category are head and neck squamous cell carcinoma, cervical cancer, Merkel cell carcinoma and many others. Viruses associated with cancer include HBV, HCV, HPV (cervical cancer, oropharyngeal cancer), and EBV (Burkitts lymphomas, gastric cancer, Hodgkin's lymphoma, other EBV positive B cell lymphomas, nasopharyngeal carcinoma and post transplant lymphoproliferative disease). The International Agency for Research on Cancer (Monograph 100B) identified the following major cancer sites associated with infectious agents:
Stomach/Gastric: Helicobacter pylori
Liver: Hepatitis B virus, hepatitis C virus (HCV), Opisthorchis viverrini, Clonorchis sinensis
Cervix uteri: Human papillomavirus (HPV) with or without HIV
Anogenital (penile, vulva, vagina, anus): HPV with or without HIV
Nasopharynx: Epstein-Barr virus (EBV)
Oropharynx: HPV with or without tobacco or alcohol consumption
Kaposi's sarcoma: Human herpes virus type 8 with or without HIV
Non-Hodgkin lymphoma: H. pylori, EBV with or without HIV, HCV, human T-cell lymphotropic virus type 1
Hodgkin's lymphoma: EBV with or without HIV
Bladder: Schistosoma haematobium.

Antibodies according to the present invention may be used for treating cancer associated with or induced by any of these infectious agents, such as the cancers specified above.

Stimulation of effector T cell response can also contribute to immunity against infectious disease and/or to recovery from infectious disease in a patient. Thus, an anti-ICOS antibody may be used for treating infectious disease by administering the antibody to a patient.

Infectious diseases include those caused by pathogens, e.g., bacterial, fungal, viral or protozoal pathogens, and treatment may be to promote immune response in a patient against the pathogen infection. An example of a bacterial pathogen is tuberculosis. Examples of viral pathogens are hepatitis B and HIV. Examples of protozoal pathogens are Plasmodium species, which cause malaria, such as P. falciparum.

The antibody may be used for treating infections, e.g., infection by any pathogen mentioned herein. Infection may be persistent or chronic infection. Infection may be localised or systemic. Extended contact between a pathogen and the immune system may lead to exhaustion of the immune system or development of tolerance (manifested for example through increased levels of Tregs, and tipping of the Treg:Teff balance in favour of Tregs) and/or to immune evasion by the pathogen, through evolution and modification of displayed pathogen antigens. These features reflect similar processes that are believed to occur in cancer. Anti-ICOS antibodies present a therapeutic approach to treating infection by a pathogen, e.g., chronic infection, through modulation of the Treg:Teff ratio in favour of Teff and/or other effects described herein.

Treatment may be of patients who have been diagnosed as having an infectious disease or an infection. Alternatively, treatment may be preventative, and administered to a patient to guard against contracting a disease, e.g., as a vaccine, as described elsewhere herein.

It has also been proposed that an immune response, particularly an IFNγ-dependent systemic immune response, could be beneficial for treatment of Alzheimer's disease and other CNS pathologies that share a neuroinflammatory component as part [30]. WO2015/136541 proposed treatment of Alzheimer's disease using an anti-PD-1 antibody. Anti-ICOS antibodies may be used in the treatment of Alzheimer's disease or other neurodegenerative diseases, optionally in combination with one or more other immunomodulators (e.g., antibody to PD-1). ³⁰ Baruch K. et al. PD-1 immune checkpoint blockade reduces pathology and improves memory in mouse models of Alzheimer's disease. Nat Med 22(2):137-137 2016

### 1.6.13. Combination Therapy

Treatment with an immunomodulatory antibody such as anti-CTLA4, anti-PD1 or anti-PDL1, especially one with Fc effector function, may create an environment in which further depletion of ICOS highly expressing immune-suppressive cells is beneficial. It may be advantageous to combine an anti-ICOS antibody with such an immunomodulator to enhance its therapeutic effects.

A patient who has been treated with an immunomodulatory antibody (e.g., anti-PDL-1, anti-PD-1, anti-CTLA-4) may particularly benefit from treatment with an anti-ICOS antibody. One reason for this is that an immunomodulatory antibody may increase the number of ICOS-positive Tregs (e.g., intratumoural Tregs) in the patient. This effect is also observed with certain other therapeutic agents, such as recombinant IL-2. Anti-ICOS antibody may reduce and/or reverse a surge or rise in ICOS+ Tregs (e.g., intratumoural Tregs) resulting from treatment of the patient with another therapeutic agent. A patient selected for treatment with an anti-ICOS antibody may thus be one who has already received treatment with a first therapeutic agent, the first therapeutic agent being an antibody (e.g., immunomodulator antibody) or other agent (e.g., IL-2) that increases the number of ICOS+ Tregs in the patient.

Immunomodulators with which an anti-ICOS antibody may be combined include antibodies to any of: PDL1 (e.g., avelumab), PD-1 (e.g., pembrolizumab or nivolumab) or CTLA-4 (e.g., ipilimumab or tremelimumab). An anti-ICOS antibody may be combined with pidilizumab. In other embodiments, an anti-ICOS antibody is not administered in combination with anti-CTLA-4 antibody, and/or optionally is administered in combination with a therapeutic antibody that is not an anti-CTLA-4 antibody.

For example, an anti-ICOS antibody may be used in combination therapy with an anti-PDL1 antibody. Preferably, the anti-ICOS antibody is one that mediates ADCC, ADCP and/or CDC. Preferably, the anti-PDL1 antibody is one that mediates ADCC, ADCP and/or CDC. An example of such combination therapy is administration of an anti-ICOS antibody with an anti-PDL1 antibody wherein both antibodies have effector positive constant regions. Thus, the anti-ICOS antibody and the anti-PDL1 antibody may both be able to mediate ADCC, CDC and/or ADCP. Fc effector function and selection of constant regions is described in detail elsewhere herein, but as one example an anti-ICOS human IgG1 may be combined with an anti-PD-L1 human IgG1. The anti-ICOS antibody and/or the anti-PD-L1 antibody may comprise a wild type human IgG1 constant region. Alternatively, the effector positive constant region of an antibody may be one that is engineered for enhanced effector function, e.g., enhanced CDC, ADCC and/or ADCP. Example antibody constant regions, including wild type human IgG1 sequences and mutations that alter effector function, are discussed in detail elsewhere herein.

Anti-PDL1 antibodies with which an anti-ICOS antibody may be combined include:
Anti-PDL1 antibody that inhibits binding of PD-1 to PDL1 and/or inhibits PDL1, optionally as effector positive human IgG1;
Anti-PD-1 antibody that inhibits binding of PD-1 to PDL1 and/or PDL2;
Avelumab, a human IgG1 antibody which inhibits PD-1 binding to PDL-1. See WO2013/079174;
Durvalumab (or “MEDI4736”), a variant human IgG1 antibody having mutations L234A, L235A and 331. See WO2011/066389;
Atezolizumab, a variant human IgG1 antibody having mutations N297A, D356E and L358M. See US2010/0203056;
BMS-936559, a human IgG4 antibody comprising mutation S228P. See WO2007/005874.

In some embodiments, the anti-PD-L1 antibody comprises atezolizumab. In some embodiments, the anti-PD-L1 antibody is atezolizumab.

Numerous further examples of anti-PD-L1 antibodies are disclosed herein and others are known in the art. Characterisation data for many of the anti-PD-L1 antibodies mentioned here has been published in U.S. Pat. Nos. 9,567,399 and 9,617,338, both incorporated by reference herein. Example anti-PD-L1 antibodies have VH and/or VL domains comprising the HCDRs and/or LCDRs of any of 1D05, 84G09, 1D05 HC mutant 1, 1D05 HC mutant 2, 1D05 HC mutant 3, 1D05 HC mutant 4, 1D05 LC mutant 1, 1D05 LC mutant 2, 1D05 LC mutant 3, 411B08, 411C04, 411D07, 385F01, 386H03, 389A03, 413D08, 413G05, 413F09, 414B06 or 416E01 as set out in U.S. Pat. No. 9,567,399 or U.S. Pat. No. 9,617,338. The antibody may comprise the VH and VL domain of any of these antibodies, and may optionally comprise a heavy and/or light chain having the heavy and/or light chain amino acid sequence of any of these antibodies. VH and VL domains of these anti-PD-L1 antibodies are further described elsewhere herein.

Further example anti-PD-L1 antibodies have VH and/or VL domains comprising the HCDRs and/or LCDRs of KN-035, CA-170, FAZ-053, M7824, ABBV-368, LY-3300054, GNS-1480, YW243.55.S70, REGN3504, or of an anti-PD-L1 antibody disclosed in any of WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 and WO2007/005874. The antibody may comprise the VH and VL domain of any of these antibodies, and may optionally comprise a heavy and/or light chain having the heavy and/or light chain amino acid sequence of any of these antibodies. The anti-ICOS antibody which is used in combination therapy with anti-PD-L1 may be an antibody of the present invention as disclosed herein. Alternatively, the anti-ICOS antibody may comprise the CDRs of, or a VH and/or VL domain of, an anti-ICOS antibody disclosed in any of the following publications: WO2016154177, US2016304610—for example any of antibodies 7F12, 37A10, 35A9, 36E10, 16G10, 37A10S713, 37A10S714, 37A10S715, 37A10S716, 37A10S717, 37A10S718, 16G10S71, 16G10S72, 16G10S73, 16G10S83, 35A9S79, 35A9S710, or 35A9S89;
WO16120789, US2016215059—for example the antibody known as 422.2 and/or H2L5; WO14033327, EP2892928, US2015239978—for example the antibody known as 314-8 and/or produced from hybridoma CNCM I-4180;
WO12131004, EP2691419, U.S. Pat. No. 9,376,493, US20160264666—for example the antibody Icos145-1 and/or antibody produced by hybridoma CNCM I-4179;
WO10056804—for example the antibody JMAb 136 or “136”; WO9915553, EP1017723B1, U.S. Pat. Nos. 7,259,247, 7,132,099, 7,125,551, 7,306,800, 7,722,872, WO05103086, EP1740617, U.S. Pat. Nos. 8,318,905, 8,916,155—for example the antibody MIC-944 or 9F3;
WO983821, U.S. Pat. No. 7,932,358B2, US2002156242, EP0984023, EP1502920, U.S. Pat. Nos. 7,030,225, 7,045,615, 7,279,560, 7,226,909, 7,196,175, 7,932,358, 8,389,690, WO02070010, EP1286668, EP1374901, U.S. Pat. Nos. 7,438,905, 7,438,905, WO0187981, EP1158004, U.S. Pat. No. 6,803,039, U.S. Pat. Nos. 7,166,283, 7,988,965, WO0115732, EP1125585, U.S. Pat. Nos. 7,465,445, 7,998,478—for example any JMAb antibody, e.g., any of JMAb-124, JMAb-126, JMAb-127, JMAb-128, JMAb-135, JMAb-136, JMAb-137, JMAb-138, JMAb-139, JMAb-140, JMAb-141, e.g., JMAb136;
WO2014/089113—for example antibody 17G9;
WO12174338;
US2016145344;
WO11020024, EP2464661, US2016002336, US2016024211, U.S. Pat. No. 8,840,889;
U.S. Pat. No. 8,497,244.

The anti-ICOS antibody optionally comprises the CDRs of 37A10S713 as disclosed in WO2016154177. It may comprise the VH and VL domains of 37A10S713, and may optionally have the antibody heavy and light chains of 37A10S713.

Combination of an anti-ICOS antibody with an immunomodulator may provide an increased therapeutic effect compared with monotherapy, and may allow therapeutic benefit to be achieved with a lower dose of the immunomodulator(s). Thus, for example, an antibody (e.g., anti-PD-L1 antibody, optionally ipilimumab or atezolizumab) that is used in combination with anti-ICOS antibody may be dosed at 3 mg/kg rather than a more usual dose of 10 mg/kg. The administration regimen of the anti-PD-L1 antibody or other antibody may involve intravenous administration over a 90 minute period every 3 weeks for a total of 4 doses.

An anti-ICOS antibody may be used to increase the sensitivity of a tumour to treatment with an anti-PD-L1 antibody, which may be recognised as a reduction in the dose at which the anti-PD-L1 antibody exerts a therapeutic benefit. Thus, anti-ICOS antibody may be administered to a patient to reduce the dose of anti-PD-L1 antibody effective to treat cancer or a tumour in the patient. Administration of anti-ICOS antibody may reduce the recommended or required dosage of anti-PD-L1 antibody administration to that patient to, for example, 75%, 50%, 25%, 20%, 10% or less, compared with the dosage when anti-PD-L1 antibody is administered without anti-ICOS. The patient may be treated by administration of anti-ICOS antibody and anti-PD-L1 antibody in a combination therapy as described herein.

The benefit of combining anti-PD-L1 with anti-ICOS may extend to a reduction in dosage of each agent when compared with its use as a monotherapy. Anti-PD-L1 antibody may be used to reduce the dose at which anti-ICOS antibody exerts a therapeutic benefit, and thus may be administered to a patient to reduce the dose of anti-ICOS antibody effective to treat cancer or a tumour in the patient. Thus, an anti-PD-L1 antibody may reduce the recommended or required dosage of anti-ICOS antibody administration to that patient to, for example, 75%, 50%, 25%, 20%, 10% or less, compared with the dosage when anti-ICOS antibody is administered without anti-PD-L1. The patient may be treated by administration of anti-ICOS antibody and anti-PD-L1 antibody in a combination therapy as described herein.

As discussed elsewhere herein, treatment with anti-PD-L1 antibody, especially antibody with effector positive Fc, appears not to increase the expression of ICOS on Teff cells. This is advantageous when administering such antibodies in combination with effector positive anti-ICOS antibodies, where an increase in ICOS expression on Teffs would undesirably render these cells more sensitive to depletion by the anti-ICOS antibody. In a combination with anti-PD-L1, anti-ICOS therapy may thus exploit a differential expression of ICOS on Teffs compared with Tregs, preferentially targeting the ICOS-high Tregs for depletion. This in turn relieves the suppression of TEffs and has a net effect of promoting the effector T cell response in a patient. The effect of targeting immune checkpoint molecules on expression of ICOS on T cells has also been studied previously—see Figure S6C in ref. [31](supplementary materials), where treatment with CTLA-4 antibody and/or anti-PD-1 antibody was reported to increase the percentage of CD4+ Tregs expressing ICOS. The effect of a therapeutic agent on ICOS expression in Tregs and Teffs may be a factor in selection of appropriate agents for use in combination with anti-ICOS antibodies, noting that effect of the anti-ICOS antibody may be enhanced under conditions where there is high differential expression of ICOS on Tregs versus Teffs. ³¹ Curran et al., PD01 and CTLA-4 combination blockade expands infiltrating T cells and reduces regulatory T and myeloid cells within B16 melanoma tumours, PNAS 107(9):4275-4280 2010

As described herein, a single dose of anti-ICOS antibody may be sufficient to provide therapeutic effect, especially in combination with other therapeutic agents such as anti-PD-L1 antibody. In tumour therapy, the underlying rationale for this single dose benefit may be that the anti-ICOS antibody mediates its effect, at least in part, by resetting or altering the microenvironment of the tumour sufficiently to render the tumour more sensitive to immune attack and/or to the effects of other immunomodulators such as those mentioned. Tumour microenvironment resetting is triggered through for example depletion of ICOS positive tumour infiltrating T-regs. So, for example, a patient may be treated with a single dose of an anti-ICOS antibody followed by one or multiple doses of anti-PD-L1 antibody. Over a period of treatment, for example six months or a year, the anti-ICOS antibody may be administered in a single dose while other agents, e.g., anti-PD-L1 antibody, are optionally administered multiple times over that treatment period, preferably with at least one such dose being administered subsequent to treatment with the anti-ICOS antibody.

Further examples of combination therapy include combination of anti-ICOS antibody with:
an antagonist of an adenosine A2A receptor (“A2AR inhibitor”);
a CD137 agonist (e.g., agonist antibody);
an antagonist of the enzyme indoleamine-2,3 dioxygenase, which catalyses the breakdown of tryptophan (“IDO inhibitor”). IDO is an immune checkpoint, activated in dendritic cells and macrophages, which contributes to immune suppression/tolerance.

Anti-ICOS antibodies may be used in combination therapy with IL-2 (e.g., recombinant IL-2 such as aldesleukin). The IL-2 may be administered at high dose (HD). Typical HD IL-2 therapy involves bolus infusion of over 500,000 IU/kg, e.g., bolus infusions of 600,000 or 720,000 IU/kg, per cycle of therapy, where 10-15 such bolus infusions are given at intervals of between 5-10 hours, e.g., up to 15 bolus infusions every 8 hours, and repeating the therapy cycle approximately every 14 to 21 days for up to 6 to 8 cycles. HD IL-2 therapy has been successful in treating tumours, especially melanoma (e.g., metastatic melanoma) and renal cell carcinoma, but its use is limited to the high toxicity of IL-2 which can cause severe adverse effects.

Treatment with high dose IL-2 has been shown to increase the population of ICOS-positive Tregs in cancer patients [32]. This increase in ICOS+ TRegs following the first cycle of HD IL-2 therapy was reported to correlate with worse clinical outcome—the higher the number of ICOS+ Tregs, the worse the prognosis. An IL-2 variant F42K has been proposed as an alternative therapy to avoid this undesirable increase in ICOS+ Treg cells [33]. However, another approach would be to exploit the increase in ICOS+ T regs by using an antibody in accordance with the present invention as a second-line therapeutic agent. ³² Sim et al., IL-2 therapy promotes suppressive ICOS+ Treg expansion in melanoma patients, J Clin Invest 2014³³ Sim et al., IL-2 variant circumvents ICOS+ regulatory T cell expansion and promotes NK cell activation, Cancer Immunol Res 2016

It may be beneficial to combine IL-2 therapy with anti-ICOS antibodies, capitalising on the ability of anti-ICOS antibodies to target TRegs that highly express ICOS, inhibiting these cells and improving the prognosis for patients undergoing IL-2 therapy. Concomitant administration of IL-2 and anti-ICOS antibody may increase the response rate while avoiding or reducing adverse events in the treated patient population. The combination may permit IL-2 to be used at lower dose compared with IL-2 monotherapy, reducing the risk or level of adverse events arising from the IL-2 therapy, while retaining or enhancing clinical benefit (e.g., reduction of tumour growth, clearance of solid tumour and/or reduction of metastasis). In this way, addition of anti-ICOS can improve treatment of patients who are receiving IL-2, whether high-dose (HD) or low-dose (LD) IL-2.

Accordingly, one aspect of the invention provides a method of treating a patient by administering an anti-ICOS antibody to the patient, wherein the patient is also treated with IL-2, e.g., HD IL-2. Another aspect of the invention is an anti-ICOS antibody for use in treating a patient, wherein the patient is also treated with IL-2, e.g., HD IL-2. The anti-ICOS antibody may be used as a second-line therapy. Thus, the patient may be one who has been treated with IL-2, e.g., having received at least one cycle of HD IL-2 therapy, and who has an increased level of ICOS+ Tregs. Assays may be performed on samples of cancer cells, e.g., tumour biopsy samples, using immunohistochemistry or FACS as described elsewhere herein to detect cells positive for ICOS, Foxp3, ICOSL and optionally one or more further markers of interest. Methods may comprise determining that the patient has an increased level of ICOS+ Tregs (e.g., in peripheral blood, or in a tumour biopsy) following IL-2 treatment, where an increased level is indicative that the patient would benefit from treatment with the anti-ICOS antibody. The increase in Tregs may be relative to control (untreated) individuals or to the patient prior to IL-2 therapy. Such patients with elevated Tregs represent a group who may not benefit from continued IL-2 treatment alone, but for whom a combination of anti-ICOS antibody and IL-2 therapy, or treatment with anti-ICOS antibody alone, offers therapeutic benefit. Thus, following a positive determination that the patient has an increased level of ICOS+ Tregs, anti-ICOS antibody and/or further IL-2 therapy may be administered. Treatment with the anti-ICOS antibody may selectively target and deplete the ICOS+ Tregs relative to other T cell populations in such patients. This provides a therapeutic effect by relieving the immunosuppression mediated by these cells and thereby enhancing activity of Teffs against the target cells, e.g., tumour cells or infected cells.

Combination therapy with anti-ICOS antibodies and IL-2 may be used for any therapeutic indication described herein, and particularly for treating a tumour, e.g., melanoma such as metastatic melanoma, or renal cell carcinoma. Thus, in one example, the patient treated with an anti-ICOS antibody is one who presents with metastatic melanoma and has been treated with IL-2, e.g., HD IL-2 therapy or LD IL-2 therapy.

In general, where an anti-ICOS antibody is administered to a patient who has received treatment with a first therapeutic agent (e.g., immunomodulator antibody) or other agent (e.g., IL-2), the anti-ICOS antibody may be administered after a minimum period of, for example, 24 hours, 48 hours, 72 hours, 1 week or 2 weeks following administration of the first therapeutic agent. The anti-ICOS antibody may be administered within 2, 3, 4 or 5 weeks after administration of the first therapeutic agent. This does not exclude additional administrations of either agent at any time, although it may be desirable to minimise the number of treatments administered, for ease of compliance for patients and to reduce costs. Rather, the relative timing of the administrations will be selected to enhance their combined effect, the first therapeutic agent creating an immunological environment (e.g., elevated ICOS+ Tregs, or antigen release as discussed below) in which the effect of the anti-ICOS antibody is especially advantageous. Thus, sequential administration of the first therapeutic agent and then the anti-ICOS antibody may allow time for the first agent to act, creating in vivo conditions in which the anti-ICOS antibody can exhibit its enhanced effect. Various administration regimens, including simultaneous or sequential combination treatments, are described herein and can be utilised as appropriate. Where the first therapeutic agent is one that increases the number of ICOS+ Tregs in the patient, the treatment regimen for the patient may comprise determining that the patient has an increased number of ICOS+ Tregs, and then administering the anti-ICOS antibody.

As noted, use of anti-ICOS antibodies in combination therapy may provide advantages of reducing the effective dose of the therapeutic agents and/or countering adverse effects of therapeutic agents that increase ICOS+ Tregs in patients. Yet further therapeutic benefits may be achieved through selecting a first therapeutic agent that causes release of antigens from target cells through “immunological cell death”, and administering the first therapeutic agent in combination with an anti-ICOS antibody. As noted, administration of the anti-ICOS antibody may sequentially follow administration of the first therapeutic agent, administration of the two agents being separated by a certain time window as discussed above.

Immunological cell death is a recognised mode of cell death, contrasting with apoptosis. It is characterised by release of ATP and HMGB1 from the cell and exposure of calreticulin on the plasma membrane [34, 35]. ³⁴ Kroemer et al. Immunologic Cell Death in Cancer Therapy, Ann Rev Immunol. 31:51-72 2013³⁵ Galluzzi, Zitvogel & Kroemer Canc. Imm. Res. 4:895-902 2016

Immunological cell death in a target tissue or in target cells promotes engulfment of the cell by an antigen-presenting cell, resulting in display of antigens from the target cell, which in turn induces antigen-specific Teff cells. Anti-ICOS antibody may increase the magnitude and/or duration of the Teff response by acting as an agonist of ICOS on the Teff cells. In addition, where the anti-ICOS antibody is Fc effector function enabled (e.g., a human IgG1 antibody), the anti-ICOS antibody may cause depletion of antigen-specific Tregs. Thus, through a combination of either or both of these effects, the balance between Teff and Treg cells is modulated in favour of enhancing Teff activity. Combination of an anti-ICOS antibody with a treatment that induces immunological cell death in a target tissue or cell type, such as in a tumour or in cancer cells, thereby promotes an immune response in the patient against the target tissue or cells, representing a form of vaccination in which the vaccine antigen is generated in vivo.

Accordingly, one aspect of the invention is a method of treating cancer in a patient by in vivo vaccination of the patient against their cancer cells. Another aspect of the invention is an anti-ICOS antibody for use in such a method. Anti-ICOS antibodies may be used in a method comprising:
treating the patient with a therapy that causes immunological cell death of the cancer cells, resulting in presentation of antigen to antigen-specific effector T cells, and
administering an anti-ICOS antibody to the patient, wherein the anti-ICOS antibody enhances the antigen-specific effector T cell response against the cancer cells.

Treatments that induce immunological cell death include radiation (e.g., ionising irradiation of cells using UVC light or γ rays), chemotherapeutic agents (e.g., oxaliplatin, anthracyclines such as doxorubicin, idarubicin or mitoxantrone, BK channel agonists such as phloretin or pimaric acid, bortezomib, cardiac glycosides, cyclophosphamide, GADD34/PP1 inhibitors with mitomycin, PDT with hypericin, polyinosinic-polycytidylic acid, 5-fluorouracil, gemcitabine, gefitnib, erlotinib, or thapsigargin with cisplatin) and antibodies to tumour-associated antigens. The tumour-associated antigen can be any antigen that is over-expressed by tumour cells relative to non-tumour cells of the same tissue, e.g., HER2, CD20, EGFR. Suitable antibodies include herceptin (anti-HER2), rituximab (anti-CD20), or cetuximab (anti-EGFR).

Thus, in some embodiments, it is advantageous to combine an anti-ICOS antibody with one or more such treatments. Optionally, the anti-ICOS antibody is adminstered to a patient who has already received such treatment. The anti-ICOS antibody may be administered after a period of, for example, 24 hours, 48 hours, 72 hours, 1 week or 2 weeks following the treatment that induces immunological cell death, e.g., between 24 to 72 hours after the treatment. The anti-ICOS antibody may be administered within 2, 3, 4 or 5 weeks after the treatment. Other regimens for combination therapy are discussed elsewhere herein.

While “in vivo vaccination” has been described above, it is also possible to treat tumour cells to induce immunological cell death ex vivo, after which the cells may be reintroduced to the patient. Rather than administering the agent or treatment that induces immunological cell death directly to the patient, the treated tumour cells are administered to the patient. Treatment of the patient may be in accordance with administration regimens described above.

As already noted, a single dose of an anti-ICOS antibody may be sufficient to provide therapeutic benefit. Thus, in the methods of treatment described herein, the anti-ICOS antibody is optionally administered as a single dose. A single dose of anti-ICOS antibody may deplete Tregs in a patient, with consequent beneficial effects in diseases such as cancer. It has previously been reported that transient ablation of Tregs has anti-tumour effects, including reducing tumour progression, treating established tumours and metastases and extending survival, and that it can enhance the therapeutic effect of tumour irradiation [36]. Administration of a single dose of anti-ICOS may provide such Treg depletion, and may be used to enhance the effects of other therapeutic approaches used in combination, such as radiotherapy. ³⁶ Bos et al., Transient regulatory T cell ablation deters oncogene-driven breast cancer and enhances radiotherapy, J Exp Med 210(11):2434-2446 2013

### 1.6.14. Antibodies to PD-L1

An antibody to PD-L1 for use in combination with an anti-ICOS antibody, whether as a separate therapeutic agent or in a multispecific antibody as described herein, may comprise the antigen-binding site of any anti-PD-L1 antibody. Numerous examples of anti-PD-L1 antibodies are disclosed herein and others are known in the art. Characterisation data for many of the anti-PD-L1 antibodies mentioned here has been published in U.S. Pat. Nos. 9,567,399 and 9,617,338, both incorporated by reference herein.

1D05 has a heavy chain variable region (V_{H}) amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No: 526, Seq ID No:528, Seq ID No: 530, Seq ID No: 532 or Seq ID No: 534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No:46).

84G09 has a heavy chain variable (V_{H}) region amino acid sequence of Seq ID No:13, comprising the CDRH1 amino acid sequence of Seq ID No:7 (IMGT) or Seq ID No:10 (Kabat), the CDRH2 amino acid sequence of Seq ID No:8 (IMGT) or Seq ID No:11 (Kabat), and the CDRH3 amino acid sequence of Seq ID No:9 (IMGT) or Seq ID No:12 (Kabat). The heavy chain nucleic acid sequence of the VH domain is Seq ID No:14. 84G09 has a light chain variable region (V_{L}) amino acid sequence of Seq ID No:23, comprising the CDRL1 amino acid sequence of Seq ID No:17 (IMGT) or Seq ID No:20 (Kabat), the CDRL2 amino acid sequence of Seq ID No:18 (IMGT) or Seq ID No:21 (Kabat), and the CDRL3 amino acid sequence of Seq ID No:19 (IMGT) or Seq ID No:22 (Kabat). The light chain nucleic acid sequence of the V_{L} domain is Seq ID No:24. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:15 (heavy chain nucleic acid sequence Seq ID No:16). A full length light chain amino acid sequence is Seq ID No:25 (light chain nucleic acid sequence Seq ID No:26).

1D05 HC mutant 1 has a heavy chain variable (V_{H}) region amino acid sequence of Seq ID No:47, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 1 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 2 has a heavy chain variable (V_{H}) region amino acid sequence of Seq ID No:48, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 2 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 3 has a heavy chain variable (V_{H}) region amino acid sequence of Seq ID No:49, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 3 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 4 has a heavy chain variable (V_{H}) region amino acid sequence of Seq ID No:342, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 4 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 LC mutant 1 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 1 has a light chain variable region (V_{L}) amino acid sequence of Seq ID No:50, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 1 is as defined by the Kabat or IMGT systems from the V_{L} sequence of Seq ID No:50. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 2 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 2 has a light chain variable region (V_{L}) amino acid sequence of Seq ID No:51, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 3 has a heavy chain variable (V_{H}) region amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 3 has a light chain variable region (V_{L}) amino acid sequence of Seq ID No:298, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 3 is as defined by the Kabat or IMGT systems from the V_{L} sequence of Seq ID No:298. The light chain nucleic acid sequence of the V_{L} domain is Seq ID No:44. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No:46).

411B08 has a heavy chain variable (V_{H}) region amino acid sequence of Seq ID No:58, comprising the CDRH1 amino acid sequence of Seq ID No:52 (IMGT) or Seq ID No:55 (Kabat), the CDRH2 amino acid sequence of Seq ID No:53 (IMGT) or Seq ID No:56 (Kabat), and the CDRH3 amino acid sequence of Seq ID No:54 (IMGT) or Seq ID No:57 (Kabat). The heavy chain nucleic acid sequence of the V_{H} domain is Seq ID No:59. 411B08 has a light chain variable region (V_{L}) amino acid sequence of Seq ID No:68, comprising the CDRL1 amino acid sequence of Seq ID No:62 (IMGT) or Seq ID No:65 (Kabat), the CDRL2 amino acid sequence of Seq ID No:63 (IMGT) or Seq ID No:66 (Kabat), and the CDRL3 amino acid sequence of Seq ID No:64 (IMGT) or Seq ID No:67 (Kabat). The light chain nucleic acid sequence of the V_{L} domain is Seq ID No:69. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:60 (heavy chain nucleic acid sequence Seq ID No:61). A full length light chain amino acid sequence is Seq ID No:70 (light chain nucleic acid sequence Seq ID No:71).

411C04 has a heavy chain variable (V_{H}) region amino acid sequence of Seq ID No:78, comprising the CDRH1 amino acid sequence of Seq ID No:72 (IMGT) or Seq ID No:75 (Kabat), the CDRH2 amino acid sequence of Seq ID No:73 (IMGT) or Seq ID No:76 (Kabat), and the CDRH3 amino acid sequence of Seq ID No:74 (IMGT) or Seq ID No:77 (Kabat). The heavy chain nucleic acid sequence of the V_{H} domain is Seq ID No:79. 411C04 has a light chain variable region (V_{L}) amino acid sequence of Seq ID No:88, comprising the CDRL1 amino acid sequence of Seq ID No:82 (IMGT) or Seq ID No:85 (Kabat), the CDRL2 amino acid sequence of Seq ID No:83 (IMGT) or Seq ID No:86 (Kabat), and the CDRL3 amino acid sequence of Seq ID No:84 (IMGT) or Seq ID No:87 (Kabat). The light chain nucleic acid sequence of the V_{L} domain is Seq ID No:89. The VH domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:80 (heavy chain nucleic acid sequence Seq ID No:81). A full length light chain amino acid sequence is Seq ID No:90 (light chain nucleic acid sequence Seq ID No:91).

411D07 has a heavy chain variable (V_{H}) region amino acid sequence of Seq ID No:98, comprising the CDRH1 amino acid sequence of Seq ID No:92 (IMGT) or Seq ID No:95 (Kabat), the CDRH2 amino acid sequence of Seq ID No:93 (IMGT) or Seq ID No:96 (Kabat), and the CDRH3 amino acid sequence of Seq ID No:94 (IMGT) or Seq ID No:97 (Kabat). The heavy chain nucleic acid sequence of the V_{H} domain is Seq ID No:99. 411D07 has a light chain variable region (V_{L}) amino acid sequence of Seq ID No:108, comprising the CDRL1 amino acid sequence of Seq ID No:102 (IMGT) or Seq ID No:105 (Kabat), the CDRL2 amino acid sequence of Seq ID No:103 (IMGT) or Seq ID No:106 (Kabat), and the CDRL3 amino acid sequence of Seq ID No:104 (IMGT) or Seq ID No:107 (Kabat). The light chain nucleic acid sequence of the V_{L} domain is Seq ID No:109. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:100 (heavy chain nucleic acid sequence Seq ID No:101). A full length light chain amino acid sequence is Seq ID No: 110 (light chain nucleic acid sequence Seq ID No:111).

385F01 has a heavy chain variable (V_{H}) region amino acid sequence of Seq ID No:118, comprising the CDRH1 amino acid sequence of Seq ID No:112 (IMGT) or Seq ID No:115 (Kabat), the CDRH2 amino acid sequence of Seq ID No:113 (IMGT) or Seq ID No:116 (Kabat), and the CDRH3 amino acid sequence of Seq ID No:114 (IMGT) or Seq ID No:117 (Kabat). The heavy chain nucleic acid sequence of the V_{H} domain is Seq ID No:119. 385F01 has a light chain variable region (V_{L}) amino acid sequence of Seq ID No:128, comprising the CDRL1 amino acid sequence of Seq ID No:122 (IMGT) or Seq ID No:125 (Kabat), the CDRL2 amino acid sequence of Seq ID No:123 (IMGT) or Seq ID No:126 (Kabat), and the CDRL3 amino acid sequence of Seq ID No:124 (IMGT) or Seq ID No:127 (Kabat). The light chain nucleic acid sequence of the V_{L} domain is Seq ID No:129. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:120 (heavy chain nucleic acid sequence Seq ID No:121). A full length light chain amino acid sequence is Seq ID No:130 (light chain nucleic acid sequence Seq ID No:131).

386H03 has a heavy chain variable (V_{H}) region amino acid sequence of Seq ID No:158, comprising the CDRH1 amino acid sequence of Seq ID No:152 (IMGT) or Seq ID No:155 (Kabat), the CDRH2 amino acid sequence of Seq ID No:153 (IMGT) or Seq ID No:156 (Kabat), and the CDRH3 amino acid sequence of Seq ID No:154 (IMGT) or Seq ID No:157 (Kabat). The heavy chain nucleic acid sequence of the V_{H} domain is Seq ID No:159. 386H03 has a light chain variable region (V_{L}) amino acid sequence of Seq ID No:168, comprising the CDRL1 amino acid sequence of Seq ID No:162 (IMGT) or Seq ID No:165 (Kabat), the CDRL2 amino acid sequence of Seq ID No:163 (IMGT) or Seq ID No:166 (Kabat), and the CDRL3 amino acid sequence of Seq ID No:164 (IMGT) or Seq ID No:167 (Kabat). The light chain nucleic acid sequence of the V_{L} domain is Seq ID No:169. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:160 (heavy chain nucleic acid sequence Seq ID No:161). A full length light chain amino acid sequence is Seq ID No:170 (light chain nucleic acid sequence Seq ID No:171).

389A03 has a heavy chain variable (VH) region amino acid sequence of Seq ID No:178, comprising the CDRH1 amino acid sequence of Seq ID No:172 (IMGT) or Seq ID No:175 (Kabat), the CDRH2 amino acid sequence of Seq ID No:173 (IMGT) or Seq ID No:176 (Kabat), and the CDRH3 amino acid sequence of Seq ID No:174 (IMGT) or Seq ID No:177 (Kabat). The heavy chain nucleic acid sequence of the V_{H} domain is Seq ID No:179. 389A03 has a light chain variable region (V_{L}) amino acid sequence of Seq ID No:188, comprising the CDRL1 amino acid sequence of Seq ID No:182 (IMGT) or Seq ID No:185 (Kabat), the CDRL2 amino acid sequence of Seq ID No:183 (IMGT) or Seq ID No:186 (Kabat), and the CDRL3 amino acid sequence of Seq ID No:184 (IMGT) or Seq ID No: 187 (Kabat). The light chain nucleic acid sequence of the V_{L} domain is Seq ID No:189. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:180 (heavy chain nucleic acid sequence Seq ID No:181). A full length light chain amino acid sequence is Seq ID No:190 (light chain nucleic acid sequence Seq ID No:191).

413D08 has a heavy chain variable (V_{H}) region amino acid sequence of Seq ID No:138, comprising the CDRH1 amino acid sequence of Seq ID No:132 (IMGT) or Seq ID No:135 (Kabat), the CDRH2 amino acid sequence of Seq ID No:133 (IMGT) or Seq ID No:136 (Kabat), and the CDRH3 amino acid sequence of Seq ID No:134 (IMGT) or Seq ID No:137 (Kabat). The heavy chain nucleic acid sequence of the V_{H} domain is Seq ID No:139. 413D08 has a light chain variable region (V_{L}) amino acid sequence of Seq ID No:148, comprising the CDRL1 amino acid sequence of Seq ID No:142 (IMGT) or Seq ID No:145 (Kabat), the CDRL2 amino acid sequence of Seq ID No:143 (IMGT) or Seq ID No:146 (Kabat), and the CDRL3 amino acid sequence of Seq ID No:144 (IMGT) or Seq ID No:147 (Kabat). The light chain nucleic acid sequence of the V_{L} domain is Seq ID No:149. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No: 140 (heavy chain nucleic acid sequence Seq ID No:141). A full length light chain amino acid sequence is Seq ID No:150 (light chain nucleic acid sequence Seq ID No:151).

413G05 has a heavy chain variable (V_{H}) region amino acid sequence of Seq ID No:244, comprising the CDRH1 amino acid sequence of Seq ID No:238 (IMGT) or Seq ID No:241 (Kabat), the CDRH2 amino acid sequence of Seq ID No:239 (IMGT) or Seq ID No:242 (Kabat), and the CDRH3 amino acid sequence of Seq ID No:240 (IMGT) or Seq ID No:243 (Kabat). The heavy chain nucleic acid sequence of the V_{H} domain is Seq ID No:245. 413G05 has a light chain variable region (V_{L}) amino acid sequence of Seq ID No:254, comprising the CDRL1 amino acid sequence of Seq ID No:248 (IMGT) or Seq ID No:251 (Kabat), the CDRL2 amino acid sequence of Seq ID No:249 (IMGT) or Seq ID No:252 (Kabat), and the CDRL3 amino acid sequence of Seq ID No:250 (IMGT) or Seq ID No:253 (Kabat). The light chain nucleic acid sequence of the V_{L} domain is Seq ID No:255. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:246 (heavy chain nucleic acid sequence Seq ID No:247). A full length light chain amino acid sequence is Seq ID No:256 (light chain nucleic acid sequence Seq ID No:257).

413F09 has a heavy chain variable (V_{H}) region amino acid sequence of Seq ID No:264, comprising the CDRH1 amino acid sequence of Seq ID No:258 (IMGT) or Seq ID No:261 (Kabat), the CDRH2 amino acid sequence of Seq ID No:259 (IMGT) or Seq ID No:262 (Kabat), and the CDRH3 amino acid sequence of Seq ID No:260 (IMGT) or Seq ID No:263 (Kabat). The heavy chain nucleic acid sequence of the V_{H} domain is Seq ID No:265. 413F09 has a light chain variable region (V_{L}) amino acid sequence of Seq ID No:274, comprising the CDRL1 amino acid sequence of Seq ID No:268 (IMGT) or Seq ID No:271 (Kabat), the CDRL2 amino acid sequence of Seq ID No:269 (IMGT) or Seq ID No:272 (Kabat), and the CDRL3 amino acid sequence of Seq ID No:270 (IMGT) or Seq ID No:273 (Kabat). The light chain nucleic acid sequence of the V_{L} domain is Seq ID No:275. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:266 (heavy chain nucleic acid sequence Seq ID No:267). A full length light chain amino acid sequence is Seq ID No:276 (light chain nucleic acid sequence Seq ID No:277).

414B06 has a heavy chain variable (V_{H}) region amino acid sequence of Seq ID No:284, comprising the CDRH1 amino acid sequence of Seq ID No:278 (IMGT) or Seq ID No:281 (Kabat), the CDRH2 amino acid sequence of Seq ID No:279 (IMGT) or Seq ID No:282 (Kabat), and the CDRH3 amino acid sequence of Seq ID No:280 (IMGT) or Seq ID No:283 (Kabat). The heavy chain nucleic acid sequence of the V_{H} domain is Seq ID No:285. 414B06 has a light chain variable region (V_{L}) amino acid sequence of Seq ID No:294, comprising the CDRL1 amino acid sequence of Seq ID No:288 (IMGT) or Seq ID No:291(Kabat), the CDRL2 amino acid sequence of Seq ID No:289 (IMGT) or Seq ID No:292 (Kabat), and the CDRL3 amino acid sequence of Seq ID No:290 (IMGT) or Seq ID No:293 (Kabat). The light chain nucleic acid sequence of the V_{L} domain is Seq ID No:295. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:286 (heavy chain nucleic acid sequence Seq ID No:287). A full length light chain amino acid sequence is Seq ID No:296 (light chain nucleic acid sequence Seq ID No:297).

416E01 has a heavy chain variable region (V_{H}) amino acid sequence of Seq ID No:349, comprising the CDRH1 amino acid sequence of Seq ID No:343 (IMGT) or Seq ID No:346 (Kabat), the CDRH2 amino acid sequence of Seq ID No:344 (IMGT) or Seq ID No:347 (Kabat), and the CDRH3 amino acid sequence of Seq ID No:345 (IMGT) or Seq ID No:348 (Kabat). The heavy chain nucleic acid sequence of the V_{H} domain is Seq ID No:350. 416E01 has a light chain variable region (V_{L}) amino acid sequence of Seq ID No:359, comprising the CDRL1 amino acid sequence of Seq ID No:353 (IMGT) or Seq ID No:356 (Kabat), the CDRL2 amino acid sequence of Seq ID No:354 (IMGT) or Seq ID No:357 (Kabat), and the CDRL3 amino acid sequence of Seq ID No:355 (IMGT) or Seq ID No:358 (Kabat). The light chain nucleic acid sequence of the V_{L} domain is Seq ID No:360. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos:207, 209, 211, 213, 215, 217, 219, 221, 223, 225, 227, 229, 231, 233, 235, 237, 536 and 538. A full length heavy chain amino acid sequence is Seq ID No:351 (heavy chain nucleic acid sequence Seq ID No:352). A full length light chain amino acid sequence is Seq ID No:361 (light chain nucleic acid sequence Seq ID No:362).

In some embodiments, the anti-PD-L1 antibody comprises atezolizumab. In some embodiments, the anti-PD-L1 antibody is atezolizumab.

### 1.6.15. Antibody-Drug Conjugates

Anti-ICOS antibodies can be used as carriers of cytotoxic agents, to target Tregs. Tregs located in the tumour microenvironment (TME) strongly express ICOS (see U.S. Pat. No. 9,957,323). ICOS is more strongly expressed on intratumoural Tregs than on intratumoural Teffs or peripheral Tregs. Thus, anti-ICOS antibodies labelled with a toxic drug or pro-drug may preferentially target Tregs in the TME to deliver the toxic payload, selectively inhibiting those cells. Such targeting of cytotoxic agents provides an additional route to removing the immune suppressive effect of Tregs, thereby altering the Treg:Teff balance in favour of Teff activity and may be used as an alternative to, or in combination with, any one or more of the other therapeutic approaches discussed herein (e.g., Fc effector-mediated inhibition of Tregs, agonism of effector T cells).

Accordingly, the invention provides an anti-ICOS antibody that is conjugated to a cytotoxic drug or pro-drug. In the case of a pro-drug, the pro-drug is activatable in the TME or other target site of therapeutic activity to generate the cytotoxic agent. Activation may be in response to a trigger such as photoactivation, e.g., using near-infrared light to activate a photoabsorber conjugate [37]. Spatially-selective activation of a pro-drug further enhances the cytotoxic effect of the antibody-drug conjugate, combining with the high ICOS expression on intratumoural Tregs to provide a cytotoxic effect that is highly selective for these cells. ³⁷ Sato et al., Spatially selective depletion of tumor-associated regulatory T cells with near-infrared photoimmunotherapy, Science Translational Medicine 8(352) 2016

For use in an antibody-drug conjugate, the cytotoxic drug or pro-drug is preferably non-immunogenic and non-toxic (dormant or inactive) during circulation of the antibody-drug conjugate in the blood. Preferably the cytotoxic drug (or the pro-drug, when activated) is potent—e.g., two to four molecules of the drug may be sufficient to kill the target cell. A photoactivatable pro-drug is silicapthalocyanine dye (IRDye 700 DX), which induces lethal damage to the cell membrane after near-infrared light exposure. Cytotoxic drugs include anti-mitotic agents such as monomethyl auristatin E and microtubule inhibitors such as maytansine derivatives, e.g., mertansine, DM1, emtansine.

Conjugation of the drug (or pro-drug) to the antibody will usually be via a linker. The linker may be a cleavable linker, e.g., disulphide, hydrazone or peptide link. Cathepsin-cleavable linkers may be used, so that the drug is released by cathepsin in tumour cells. Alternatively, non-cleavable linkers can be used, e.g., thioether linkage. Additional attachment groups and/or spacers may also be included.

The antibody in the antibody-drug conjugate may be an antibody fragment, such as Fab′2 or other antigen-binding fragment as described herein, as the small size of such fragments may assist penetration to the tissue site (e.g., solid tumour).

An anti-ICOS antibody according to the present invention may be provided as an immunocytokine. Anti-ICOS antibodies may also be administered with immunocytokines in combination therapy. A number of examples of antibodies are described herein for use in combination therapy with anti-ICOS, and any of these (e.g., an anti-PD-L1 antibody) may be provided as immunocytokines for use in the present invention. An immunocytokine comprises an antibody molecule conjugated to a cytokine, such as IL-2. Anti-ICOS:IL-2 conjugates and anti-PD-L1:IL-2 conjugates are thus further aspects of the present invention.

An IL-2 cytokine may have activity at the high (αβγ) affinity IL-2 receptor and/or the intermediate affinity (αβ) IL-2 receptor. IL-2 as used in an immunocytokine may be human wild type IL-2 or a variant IL-2 cytokine having one or more amino acid deletions, substitutions or additions, e.g., IL-2 having a 1 to 10 amino acid deletion at the N-terminus. Other IL-2 variants include mutations R38A or R38Q.

An example anti-PD-L1 immunocytokine comprises an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
wherein the V_{H} domain and V_{L} domain are comprised by an antigen-binding site that specifically binds to human PD-L1; and
wherein the immunocytokine comprises a V_{H} domain which comprises a CDRH3 comprising the motif X₁GSGX₂YGX₃X₄FD (SEQ ID NO: 609), wherein X₁, X₂ and X₃ are independently any amino acid, and X₄ is either present or absent, and if present, may be any amino acid.

The VH and VL domain may be the VH and VL domain of any anti-PD-L1 antibody mentioned herein, e.g., the 1D05 VH and VL domains.

The IL-2 may be human wild type or variant IL-2.

### 1.6.16. Vaccination

Anti-ICOS antibodies may be provided in vaccine compositions or co-administered with vaccines preparations. ICOS is involved in T follicular helper cell formation and the germinal centre reaction [38]. Agonist ICOS antibodies thus have potential clinical utility as molecular adjuvants to enhance vaccine efficacy. The antibodies may be used to increase protective efficacy of numerous vaccines, such as those against hepatitis B, malaria, HIV. ³⁸ Crotty S. T follicular helper cell differentiation, function, and roles in disease. Immunity. 2014 Oct. 16; 41(4):529-42.

In the context of vaccination, the anti-ICOS antibody will generally be one that lacks Fc effector function, and thus does not mediate ADCC, CDC or ADCP. The antibody may be provided in a format lacking an Fc region, or having an effector null constant region. Optionally, an anti-ICOS antibody may have a heavy chain constant region that binds one or more types of Fc receptor but does not induce ADCC, CDC or ADCP activity, or that exhibits lower ADCC, CDC and ADCP activity compared with wild type human IgG1. Such a constant region may be unable to bind, or may bind with lower affinity, the particular Fc receptor(s) responsible for triggering ADCC, CDC or ADCP activity. Alternatively, where cellular effector functions are acceptable or desirable in the context of the vaccination, the anti-ICOS antibody may comprise a heavy chain constant region that is Fc effector function positive. Any of IgG1, IgG4 and IgG4.PE formats may for instance be used for anti-ICOS antibodies in vaccination regimens, and other examples of suitable isotypes and antibody constant regions are set out in more detail elsewhere herein.

### 1.6.17. Formulations and Administration

Antibodies may be monoclonal or polyclonal, but are preferably provided as monoclonal antibodies for therapeutic use. They may be provided as part of a mixture of other antibodies, optionally including antibodies of different binding specificity.

Antibodies according to the invention, and encoding nucleic acid, will usually be provided in isolated form. Thus, the antibodies, VH and/or VL domains, and nucleic acids may be provided purified from their natural environment or their production environment. Isolated antibodies and isolated nucleic acid will be free or substantially free of material with which they are naturally associated, such as other polypeptides or nucleic acids with which they are found in vivo, or the environment in which they are prepared (e.g., cell culture) when such preparation is by recombinant DNA technology in vitro. Optionally an isolated antibody or nucleic acid (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature.

Antibodies or nucleic acids may be formulated with diluents or adjuvants and still for practical purposes be isolated—for example they may be mixed with carriers if used to coat microtitre plates for use in immunoassays, and may be mixed with pharmaceutically acceptable carriers or diluents when used in therapy. As described elsewhere herein, other active ingredients may also be included in therapeutic preparations. Antibodies may be glycosylated, either naturally in vivo or by systems of heterologous eukaryotic cells such as CHO cells, or they may be (for example if produced by expression in a prokaryotic cell) unglycosylated. The invention encompasses antibodies having a modified glycosylation pattern. In some applications, modification to remove undesirable glycosylation sites may be useful, or e.g., removal of a fucose moiety to increase ADCC function [39]. In other applications, modification of galactosylation can be made in order to modify CDC. ³⁹ Shields et al. (2002) JBC 277:26733

Typically, an isolated product constitutes at least about 5%, at least about 10%, at least about 25%, or at least about 50% of a given sample. An antibody may be substantially free from proteins or polypeptides or other contaminants that are found in its natural or production environment that would interfere with its therapeutic, diagnostic, prophylactic, research or other use.

An antibody may have been identified, separated and/or recovered from a component of its production environment (eg, naturally or recombinantly). The isolated antibody may be free of association with all other components from its production environment, eg, so that the antibody has been isolated to an FDA-approvable or approved standard. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated antibody or its encoding nucleic acid will be prepared by at least one purification step.

The invention provides therapeutic compositions comprising the antibodies described herein. Therapeutic compositions comprising nucleic acid encoding such antibodies are also provided. Encoding nucleic acids are described in more detail elsewhere herein and include DNA and RNA, e.g., mRNA. In therapeutic methods described herein, use of nucleic acid encoding the antibody, and/or of cells containing such nucleic acid, may be used as alternatives (or in addition) to compositions comprising the antibody itself. Cells containing nucleic acid encoding the antibody, optionally wherein the nucleic acid is stably integrated into the genome, thus represent medicaments for therapeutic use in a patient. Nucleic acid encoding the anti-ICOS antibody may be introduced into human B lymphocytes, optionally B lymphocytes derived from the intended patient and modified ex vivo. Optionally, memory B cells are used. Administration of cells containing the encoding nucleic acid to the patient provides a reservoir of cells capable of expressing the anti-ICOS antibody, which may provide therapeutic benefit over a longer term compared with administration of isolated nucleic acid or isolated antibody.

Compositions may contain suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTINT™) DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311. Compositions may comprise the antibody or nucleic acid in combination with medical injection buffer and/or with adjuvant.

Antibodies, or their encoding nucleic acids, may be formulated for the desired route of administration to a patient, e.g., in liquid (optionally aqueous solution) for injection. Various delivery systems are known and can be used to administer the pharmaceutical composition of the invention. Methods of introduction include, but are not limited to, intradermal, intramuscular, intraperitoneal, intravenous, subcutaneous, intranasal, epidural, and oral routes. Formulating antibodies for subcutaneous administration typically requires concentrating them into a smaller volume compared with intravenous preparations. The high potency of antibodies according to the present invention may lend them to use at sufficiently low doses to make subcutaneous formulation practical, representing an advantage compared with less potent anti-ICOS antibodies.

The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule. A pharmaceutical composition of the present invention can be delivered subcutaneously or intravenously with a standard needle and syringe. It is envisaged that treatment will not be restricted to use in the clinic. Therefore, subcutaneous injection using a needle-free device is also advantageous. With respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPENT™, OPTIPEN PRO™, OPTIPEN STARLET™ and OPTICLIKT™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, the aforesaid antibody may be contained in about 5 to about 100 mg and in about 10 to about 250 mg for the other dosage forms.

The antibody, nucleic acid, or composition comprising it, may be contained in a medical container such as a phial, syringe, IV container or an injection device. In an example, the antibody, nucleic acid or composition is in vitro, and may be in a sterile container. In an example, a kit is provided comprising the antibody, packaging and instructions for use in a therapeutic method as described herein.

One aspect of the invention is a composition comprising an antibody or nucleic acid of the invention and one or more pharmaceutically acceptable excipients, examples of which are listed above. “Pharmaceutically acceptable” refers to approved or approvable by a regulatory agency of the USA Federal or a state government or listed in the U.S. Pharmacopeia or other generally recognized pharmacopeia for use in animals, including humans. A pharmaceutically acceptable carrier, excipient, or adjuvant can be administered to a patient, together with an agent, e.g., any antibody or antibody chain described herein, and does not destroy the pharmacological activity thereof and is nontoxic when administered in doses sufficient to deliver a therapeutic amount of the agent.

In some embodiments, an anti-ICOS antibody will be the sole active ingredient in a composition according to the present invention. Thus, a composition may consist of the antibody or it may consist of the antibody with one or more pharmaceutically acceptable excipients. However, compositions according to the present invention optionally include one or more additional active ingredients. Detailed description of agents with which the anti-ICOS antibodies may be combined is provided elsewhere herein. Optionally, compositions contain multiple antibodies (or encoding nucleic acids) in a combined preparation, e.g., a single formulation comprising the anti-ICOS antibody and one or more other antibodies. Other therapeutic agents that it may be desirable to administer with antibodies or nucleic acids according to the present invention include analgesic agents. Any such agent or combination of agents may be administered in combination with, or provided in compositions with antibodies or nucleic acids according to the present invention, whether as a combined or separate preparation. The antibody or nucleic acid according to the present invention may be administered separately and sequentially, or concurrently and optionally as a combined preparation, with another therapeutic agent or agents such as those mentioned.

Anti-ICOS antibodies for use in a particular therapeutic indication may be combined with the accepted standard of care. Thus, for anti-cancer treatment, the antibody therapy may be employed in a treatment regimen that also includes chemotherapy, surgery and/or radiation therapy for example. Radiotherapy may be single dose or in fractionated doses, either delivered to affected tissues directly or to the whole body.

Multiple compositions can be administered separately or simultaneously. Separate administration refers to the two compositions being administered at different times, e.g. at least 10, 20, 30, or 10-60 minutes apart, or 1, 2, 3, 4, 5, 6, 7, 8, 9, 10, 12 hours apart. One can also administer compositions at 24 hours apart, or even longer apart. Alternatively, two or more compositions can be administered simultaneously, e.g. less than 10 or less than 5 minutes apart. Compositions administered simultaneously can, in some aspects, be administered as a mixture, with or without similar or different time release mechanism for each of the components.

Antibodies, and their encoding nucleic acids, can be used as therapeutic agents. Patients herein are generally mammals, typically humans. An antibody or nucleic acid may be administered to a mammal, e.g., by any route of administration mentioned herein.

Administration is normally in a “therapeutically effective amount”, this being an amount that produces the desired effect for which it is administered, sufficient to show benefit to a patient. The exact amount will depend on the purpose of the treatment, and will be ascertainable by one skilled in the art using known techniques (see, for example, Lloyd (1999) The Art, Science and Technology of Pharmaceutical Compounding). Prescription of treatment, e.g. decisions on dosage etc, is within the responsibility of general practitioners and other medical doctors and may depend on the severity of the symptoms and/or progression of a disease being treated. A therapeutically effective amount or suitable dose of antibody or nucleic acid can be determined by comparing its in vitro activity and in vivo activity in an animal model. Methods for extrapolation of effective dosages in mice and other test animals to humans are known.

As indicated by the in vivo studies described in the Examples herein, anti-ICOS antibody may be effective at a range of doses, including surprisingly low doses. Surprisingly, in view of these pre-clinical studies, low doses per body weight of anti-ICOS antibodies (e.g., KY1044) or low fixed doses of anti-ICOS antibodies (e.g., KY1044) were effective at yielding partial or complete anti-tumour activity in human patients across various cancers.

Anti-ICOS antibodies (e.g., full length antibodies or antigen-binding fragments thereof) may be administered to a subject in an amount in one of the following values or ranges per dose:
about 10 μg/kg body weight to about 3 mg/kg body weight,
about 10 μg/kg body weight to about 1 mg/kg body weight,
about 10 μg/kg body weight to about 0.3 mg/kg body weight,
about 10 μg/kg body weight to about 0.1 mg/kg body weight, or
about 10 μg/kg body weight to about 30 μg/kg body weight.

For fixed dosing in adult humans, a suitable dose may be about 10 mg or lower, 9 mg or lower, or about 8 mg or lower, e.g., about 8 mg, about 7 mg, about 6 mg, about 5 mg, about 4 mg, about 3 mg, about 2.4 mg, about 2 mg, about 1 mg, about 0.8 mg, or about 0.5 mg, or any value in between. In some embodiments, the subject is administered an anti-ICOS antibody (e.g., KY1044) at about 0.5-10 mg per dose. In some embodiments, the subject is administered an anti-ICOS antibody (e.g., KY1044) at about 0.5-8 mg per dose. In some embodiments, the subject is administered an anti-ICOS antibody (e.g., KY1044) at about 0.8-8 mg per dose. In some embodiments, the subject is administered an anti-ICOS antibody (e.g., KY1044) at about 0.8-2.4 mg per dose.

In some embodiments, the subject is administered an anti-ICOS antibody (e.g., KY1044) at about 8 mg per dose. In some embodiments, the subject is administered an anti-ICOS antibody (e.g., KY1044) at about 2.4 mg per dose. In some embodiments, the subject is administered an anti-ICOS antibody (e.g., KY1044) at about 0.8 mg per dose.

In methods of treatment described herein, one or more doses may be administered. In some cases, a single dose may be effective to achieve a long-term benefit. Thus, the method may comprise administering a single dose of the antibody, its encoding nucleic acid, or the composition. Alternatively, multiple doses may be administered, usually sequentially and separated by a period of days, weeks or months. An anti-ICOS antibody may be repeatedly administered to a subject at intervals of 2 to 6 weeks, e.g., every 2 weeks, every 3 weeks, every 4 weeks, every 5 weeks, or every 6 weeks. In some embodiments, the anti-ICOS antibody is administered to a subject every 3 weeks. In some embodiments, the anti-ICOS antibody is administered to a subject every 6 weeks. In some embodiments, KY1044 is administered to a subject every 3 weeks. In some embodiments, KY1044 is administered to a subject every 6 weeks. Optionally, the anti-ICOS antibody may be administered to a subject once a month, or less frequently, e.g., every two months or every three months. Accordingly, a method of treating a patient may comprise administering a single dose of the anti-ICOS antibody to the subject, and not repeating the administration for at least one month, at least two months, at least three months, and optionally not repeating the administration for at least 12 months.

In some embodiments the anti-ICOS antibody, e.g., KY1044, is administered to the subject for at least 6 months. In some embodiments the anti-ICOS antibody, e.g., KY1044, is administered to the subject for 6 months. In some embodiments the anti-ICOS antibody, e.g., KY1044, is administered to the subject for at least 12 months. In some embodiments the anti-ICOS antibody, e.g., KY1044, is administered to the subject for 12 months. In some embodiments the anti-ICOS antibody, e.g., KY1044, is administered to the subject for longer than 12 months.

In some embodiments, KY1044 is administered to a subject every 3 weeks for at least 6 months, e.g., for 6 months, for 12 months, or for longer than 12 months.

Comparable therapeutic effects may be obtained using either one or multiple doses of anti-ICOS antibody, which may be a result of a single dose of antibody being effective to reset the tumour microenvironment. Physicians can tailor the administration regimen of the anti-ICOS antibody to the disease and the patient undergoing therapy, taking into account the disease status and any other therapeutic agents or therapeutic measures (e.g., surgery, radiotherapy etc) with which the anti-ICOS antibody is being combined. In some embodiments, an effective dose of an anti-ICOS antibody is administered more frequently than once a month, such as, for example, once every three weeks, once every two weeks, or once every week. Treatment with anti-ICOS antibody may include multiple doses administered over a period of at least a month, at least six months, or at least a year. The multiple doses may be the same or may be different.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilised (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well. In the context of the invention, treatment may be preventative treatment.

In some embodiments, “treating” comprises treating a disease or condition amenable to therapy by depleting regulatory T cells (Tregs) and/or increasing effector T cell (Teff) response. As used herein, such a disease or condition includes but is not limited to a tumour and/or a cancer. In some embodiments, the cancer is an advanced and/or metastatic cancer.

As used herein, “about” in reference to a dosing amount in mg refers to plus or minus 0.1 mg of the stated value if the stated value is less than 1.5 mg, and refers to plus or minus 0.5 mg of the stated value if the stated value is at least 1.5 mg.

### 1.6.18. T Cell Therapy

WO2011/097477 described use of anti-ICOS antibodies for generating and expanding T cells, by contacting a population of T cells with a first agent that provides a primary activation signal (e.g., an anti-CD3 antibody) and a second agent that activates ICOS (e.g., an anti-ICOS antibody), optionally in the presence of a Th17 polarising agent such as IL-1β, IL-6, neutralising anti-IFNγ and/or anti-IL-4. Anti-ICOS antibodies described herein may be used in such methods to provide T cell populations. Populations of cultured expanded T cells having therapeutic activity (e.g., anti-tumour activity) may be generated. As described in WO2011/097477, such T cells may be used therapeutically in methods of treating patients by immunotherapy.

### 1.6.19. Morphological Assay for Anti-ICOS Antibodies as Therapeutic Candidates

It was observed that when candidate therapeutic anti-ICOS antibodies were coupled to a solid surface and brought into contact with ICOS-expressing T cells, they were able to induce morphological change in the cells. On addition of ICOS+ T cells to wells that were internally coated with anti-ICOS antibodies, cells were seen to change from their initial rounded shape, adopting a spindle-shape, spreading and adhering to the antibody-coated surface. This morphological change was not observed with control antibody. Moreover, the effect was found to be dose-dependent, with faster and/or more pronounced shape change occurring as the concentration of antibody on the surface increased. The shape change provides a surrogate indicator of T cell binding to ICOS, and/or of agonism by anti-ICOS antibody. The assay may be used to identify an antibody that promotes multimerisation of ICOS on the T cell surface. Such antibodies represent therapeutic candidate agonist antibodies. Conveniently, the visual indicator provided by this assay is a simple method of screening antibodies or cells, particularly in large numbers. The assay may be automated to run in a high-throughput system.

Accordingly, one aspect of the invention is an assay for selecting an antibody that binds ICOS, optionally for selecting an ICOS agonist antibody, the assay comprising: providing an array of antibodies immobilised (attached or adhered) to a substrate in a test well;
adding ICOS-expressing cells (e.g., activated primary T cells, or MJ cells) to the test well;
observing morphology of the cells;
detecting shape change in the cells from rounded to flattened against the substrate within the well; wherein the shape change indicates that the antibody is an antibody that binds ICOS, optionally an ICOS agonist antibody, and
selecting the antibody from the test well.

The assay may be run with multiple test wells, each containing a different antibody for testing, optionally in parallel, e.g., in a 96 well plate format. The substrate is preferably an inner surface of the well. Thus, a two-dimensional surface is provided against which flattening of the cells may be observed. For example, the bottom and/or wall of a well may be coated with antibody. Tethering of antibody to the substrate may be via a constant region of the antibody.

A negative control may be included, such an antibody known not to bind ICOS, preferably an antibody that does not bind an antigen on the surface of the ICOS-expressing cells to be used. The assay may comprise quantifying the degree of morphological change and, where multiple antibodies are tested, selecting an antibody that induces greater morphological change than one or more other test antibodies.

Selection of antibody may comprise expressing nucleic acid encoding the antibody present in the test well of interest, or expressing an antibody comprising the CDRs or antigen binding domain of that antibody. The antibody may optionally be reformatted, for example to provide an antibody comprising the antigen binding domain of the selected antibody, e.g., an antibody fragment, or an antibody comprising a different constant region. A selected antibody is preferably provided with a human IgG1 constant region or other constant region as described herein. A selected antibody may further be formulated in a composition comprising one or more additional ingredients—suitable pharmaceutical formations are discussed elsewhere herein.

Various further aspects and embodiments of the present invention will be apparent to those skilled in the art in view of the present disclosure. All documents mentioned in this specification, including published US counterparts of any patents or patent applications referred to, are incorporated herein by reference in their entirety.

### 1.7. EXPERIMENTAL EXAMPLES

The generation, characterisation, and performance of anti-ICOS antibodies were previously disclosed in U.S. Pat. No. 9,957,323.

### 1.8. Example 1: Monotherapeutic Efficacy of Anti-ICOS Ab Against A20 Tumour Growth in Mouse

Anti-ICOS antibodies STIM001 mIgG2a and STIM003 mIgG2a each showed strong anti-tumour efficacy when used as monotherapies in vivo in a mouse A20 syngeneic model.

### 1.8.1. Materials and Methods

The efficacy study was performed in BALB/c mice using the sub-cutaneous A20 reticulum cell sarcoma model (ATCC, TIB-208). The A20 cell line is a BALB/c B cell lymphoma line derived from a spontaneous reticulum cell neoplasm found in an old BALB/cAnN mouse. This cell line has been reported to be positive for ICOSL.

BALB/c mice were supplied by Charles River UK>18 gram and housed under specific pathogen-free conditions. A total of 5×10e5 A20 cells (passage number below P20) were subcutaneously injected into the right flanks of mice. The A20 cells were passaged in vitro washed twice in PBS and re-suspended in RPMI supplemented with 10% foetal calf serum. Cell viability was confirmed to be above 85% at the time of tumour cell injection. Unless stated otherwise, antibody or isotype administration was initiated from day 8 post tumour cells injection.

STIM001 and STIM003 anti-ICOS antibodies were generated in mouse IgG2a isotype format. The mouse cross reactive anti-PD-L1 antibody (AbW) was also generated in the same isotype format (mouse IgG2a). STIM001, STIM003 and anti-PD-L1 antibodies were dosed intraperitoneally (IP) at 200 μg of each antibody twice a week starting from day 8 (dosing for 3 weeks between day 8-29) post tumour cell implantation. Animal weights and tumour volume were measured 3 times a week from the day of tumour cell injection. Tumour volume was calculated by use of the modified ellipsoid formula ½(Length×Width2). Mice were kept on study until their tumour reached an average diameter of 12 mm. The experiment was stopped at day 43 post tumour cell implantation. Tumour growth was monitored and compared with tumours of animals treated with isotype control (mIgG2a) antibody. Treatment groups are shown in Table E20 below.

TABLE E20
Treatment groups for A20 study.

| | Number | |
|---|---|---|
| | of | Treatment regimen |
| Group | animals | (twice per week for 3 weeks 7 doses) |
| 1 | 8 | mIgG2a isotype control 200 μg/mouse/ each dose |
| 2 | 8 | Anti-PD-Ll mIGg2a (AbW) 200 μg/mouse/ each dose |
| 3 | 8 | Anti-ICOS mIgG2a STIM001 200 μg/mouse each dose |
| 4 | 8 | Anti-ICOS mIgG2a STIM003 200 μg/mouse/ each dose |

### 1.8.2. Results

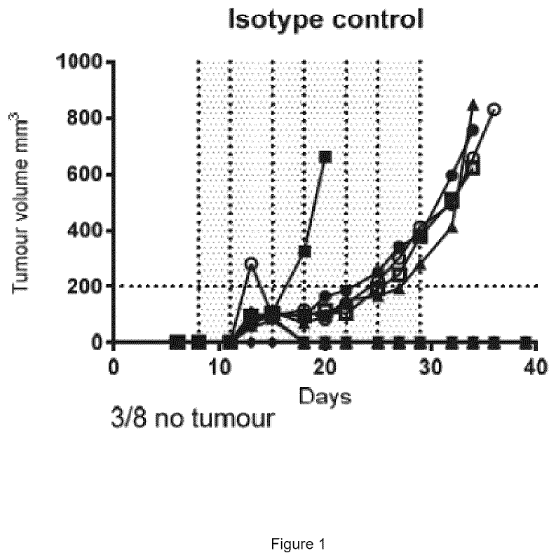

Monotherapy administration of either STIM001 or STIM003 (mIgG2a) in the A20 tumour model produced a complete anti-tumour response (FIG. 3, FIG. 4). All the animals administered with either STIM001 or STIM003 were cured of the disease. This contrasts with the results in the isotype control and PD-L1 mIgG2a groups (FIG. 1, FIG. 2). In rare cases, regression of tumours was observed for some animals in the isotype control (spontaneous regression) and anti-PDL-1 groups, but treatment with anti-ICOS antibody produced significantly greater efficacy. At the end of the study, 3 of 8 control animals and 2 of 8 anti-PDL-1 treated animals had no tumour. However, all animals treated with either STIM001 or STIM003 were tumour free at the end of the study (8 of 8 mice in both groups), representing 100% cure using the anti-ICOS antibodies.

### 1.9. Example 2: Strong Anti-Tumour Efficacy In Vivo in the J558 Myeloma Syngeneic Model for Combination of Anti-ICOS Antibody and Anti-PD-L1 Antibody

Anti-ICOS antibody STIM003 mIgG2a and anti-PD-L1 antibody AbW mIgG2a were administered individually and in combination in the J558 tumour model. This is a syngeneic mouse model of myeloma. The anti-ICOS antibody was found to inhibit tumour growth when dosed as monotherapy or in combination with anti-PD-L1.

### 1.9.1. Materials & Methods

Anti-tumour efficacy studies were performed in Balb/c mice using the sub-cutaneous J558 plasmacytoma:myeloma cell line (ATCC, TIB-6). Balb/c mice were supplied by Charles River UK at 6-8 weeks of age and >18 g and housed under specific pathogen-free conditions. A total of 5×10⁶ cells (passage number below P15) were subcutaneously injected (in 100 μl) into the right flanks of mice. Unless stated otherwise, on day 11 post tumour cells injection, the animals were randomised based on tumour size and treatments were initiated. The J558 cells were passaged in vitro by using TrypLE™ Express Enzyme (Thermofisher), washed twice in PBS and resuspended in DMEM supplemented with 10% foetal calf serum. Cell viability was confirmed to be above 90% at the time of tumour cell injection.

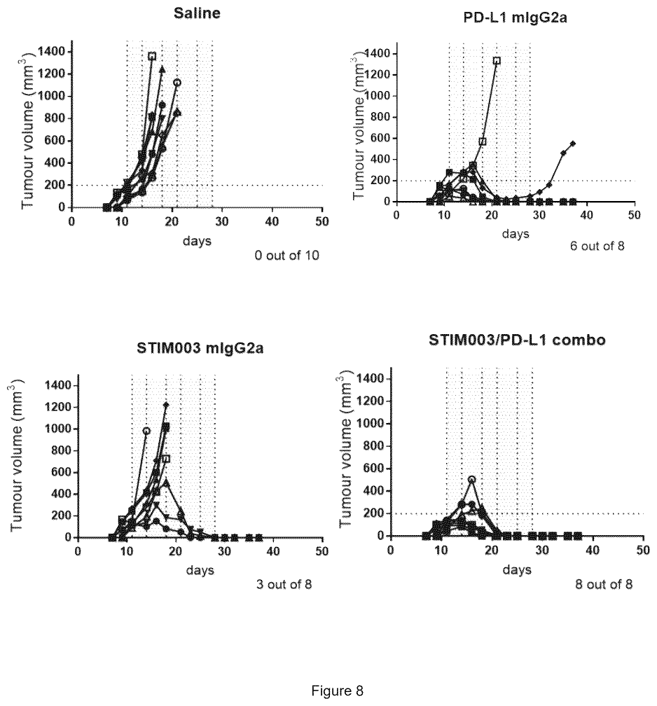

Treatment was initiated when the tumours reached an average volume of ˜140 mm\hat{ }3. Animals were then allocated to 4 groups with similar average tumour size (see Table E-21 for the dosing groups). Both antibodies, which are mouse cross-reactive, were dosed IP from day 11 (post tumour cell implantation) twice a week for 3 weeks (FIG. 8) unless the animals had to be removed from study due to welfare (rare) or tumour size. As a control, a group of animals (n=10) was dosed at the same time using a saline solution. For the combination group, both STIM003 and anti-PDL1 antibodies were dosed concurrently IP at 60 μg and 200 μg respectively (in 0.9% saline). Tumour growth was monitored over 37 days and compared to tumours of animals treated with saline. Animal weight and tumour volume were measured 3 time a week from the day of tumour cell injection. Tumour volume was calculated by use of the modified ellipsoid formula ½(Length×Width²). Mice were kept on studies until their tumour reached an average diameter of 12 mm³ or, in rare cases, when incidence of tumour ulceration was observed (welfare).

TABLE E21
Treatment groups for J558 efficacy study.

| | Number | |
|---|---|---|
| | of | |
| Groups | animals | Treatment regimen twice per week from day 11 |
| 1 | 10 | Saline |
| 2 | 8 | Anti-PD-L1 mIgG2a 200 μg (AbW) |
| 3 | 8 | Anti-ICOS STIM003 mIgG2a/anti-PD-L1 mIgG2a |
| | | (AbW) combination 60 μg/200 μg (respectively) |
| 4 | 8 | Anti-ICOS STIM003 mIgG2a 60 μg |

### 1.9.2. Results

J558 syngeneic tumours were highly aggressive and all the animals in the saline control group (n=10) had to be removed from studies by day 21 due to tumour size. The anti-STIM003 mIgG2a and the anti-PDL1 mIgG2a both demonstrated good efficacy as monotherapies in this model with 37.5% and 75% of the animals cured of disease, respectively. Importantly, combination of the two antibodies resulted in 100% of the animals having rejected the plasmacytoma tumours by day 37. Data are shown in FIG. 8.

### 1.10. Example 3: Administration of Anti-PD1 Increases ICOS Expression on TILs Significantly More than Anti-PD-L1 Antibody

A pharmacodynamic study was performed in animals harbouring established CT26 tumours to evaluate the effect of treatment with anti-PD-L1 or anti-PD-1 antibodies on ICOS expression on subsets of tumour infiltrating lymphocytes (TILs). The following antibodies were compared:
anti-PD-L1 AbW mIgG1 [limited effector function]
anti-PD-L1 AbW mIgG2a [with effector function]
anti-PD-L1 10F9.G2 rat IgG2b [with effector function]
anti-PD1 antibody RMT1-14 rat IgG2a [effector null].

Tumours of treated mice were isolated, dissociated to single cells and stained for CD45, CD3, CD4, CD8, FOXP3 and ICOS.

### 1.10.1. Materials & Methods

Rat anti-PD-1 RMP1-14 IgG2a (BioXCell; Catalog number: BE0146), rat anti-PD-L1 10F9.G2 IgG2b (Bio-Legend; Catalog number: 124325) and anti-PD-L1 AbW mIgG1 and mIgG2a were tested in the CT26 tumour model by dosing i.p. with 130 μg on days 13 and 15 post tumour cell implantation. On day 16, animals were culled and the mouse tumours were harvested for FACS analysis. Tumours were dissociated using a mouse tumour dissociation kit (Miltenyi Biotec) and homogenised. The resulting cell suspensions were clarified through 70 μM filters, pelleted and resuspended in FACS buffer at 2 million cells/well in a 96 well plate. The cell suspensions were incubated with anti-16/32 mAb (eBioscience) and stained with FACS antibodies specific for CD3 (17A2), CD45 (30-F11), CD4 (RM4-5), CD8 (53-6.7) and ICOS (7E.17G9) all obtained from eBioscience Ltd. Cells were also stained with LiveDead Yellow fixable viability dye (Life technologies). For the Foxp3 intracellular staining, samples were fixed, permeabilised, and stained with antibody specific for Foxp3 (eBioscience, FJK-16s). The samples were resuspended in PBS and data acquired on the Attune flow cytometer (Invitrogen) and analysed using FlowJo V10 software (Treestar).

### 1.10.2. Results

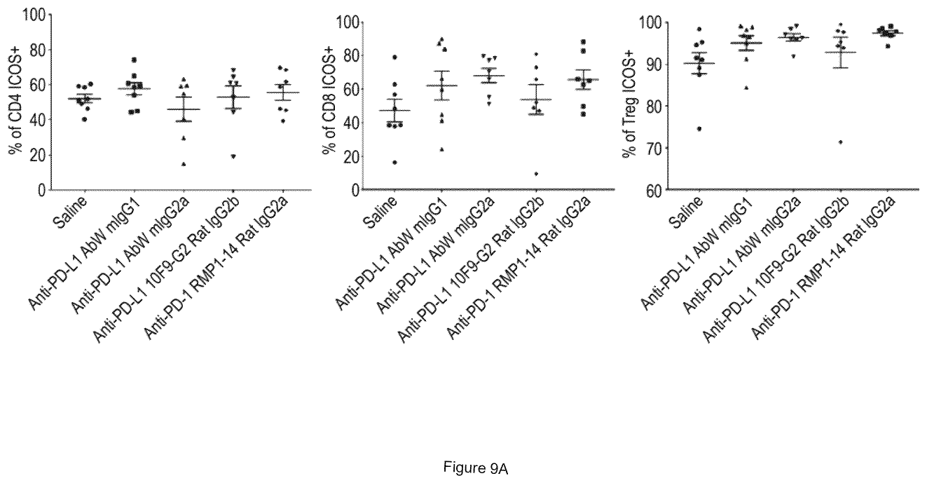

Treatment with anti-PD1 and anti-PD-L1 antibodies only resulted in a marginal increase in the percentage on CD8 cells and T Regs expressing ICOS at the measured timepoint. However, in response to anti-PD1 rat IgG2a, a clear and significant (over the saline treated group) increase in ICOS expression (increased dMFI) was observed on the surface of ICOS+ve CD8 cells. ICOS expression was also noted to be upregulated on CD4 effector and CD4 T Reg cells although this did not reach statistical significance. This anti-PD1 antibody induced a marked increase in ICOS expression on CD8 effector cells that was barely seen with the anti-PD-L1 mIgG2a. Similarly, when comparing the different formats of anti-PD-L1 antibodies, in some of the animals treated it was observed that the antibody having the lowest effector function (mIgG1) was associated with higher ICOS expression on effector CD8 and CD4 cells when compared with antibody having effector function (mIgG2a and ratIgG2b), which rarely showed this. See FIG. 9.

An increase in ICOS expression on effector CD8/CD4 T cells may have the effect of rendering these cells more sensitive to depletion by anti-ICOS antibody (e.g., on treatment of mice with STIM003 mIgG2a). An antibody that exhibits lower ICOS induction in effector CD8 and CD4 T cells may be preferable for use in combination with anti-ICOS antibody. The data from this study indicate that anti-PD-L1 effector positive antibody may be especially suitable for combination with anti-ICOS effector positive antibody, reflecting the anti-tumour efficacy observed when combining anti-PDL1 mIgG2a with STIM003 mIgG2a reported in other Examples herein.

### 1.11. Example 4: Strong Anti-Tumour Efficacy of Single Dose Anti-ICOS Antibody Monotherapy In Vivo in a B Cell Lymphoma Syngeneic Model

This experiment confirms the anti-tumour efficacy of STIM003 mIgG2a as monotherapy. Strong anti-tumour efficacy was demonstrated after short exposure of STIM003 mIgG2a.

### 1.11.1. Materials & Methods

Efficacy studies were performed in BALB/c mice using the sub-cutaneous A20 Reticulum Cell Sarcoma model (ATCC number CRL-TIB-208). BALB/c mice were supplied by Charles River UK at 6-8 weeks of age and >18 g and housed under specific pathogen-free conditions. A total of 5×10E5 A20 cells (passage number below P20) were subcutaneously injected into the right flanks of mice. Treatments were initiated at day 8 post tumour cells injection as shown in the table below. The A20 cells were passaged in vitro by using TrypLE™ Express Enzyme (Thermofisher), washed twice in PBS and resuspended in RPMI supplemented with 10% foetal calf serum. Cell viability was confirmed to be above 85% at the time of tumour cell injection. STIM003 mIgG2a was used either as a single dose (SD) of 60 μg (equivalent to 3 mg/kg for a 20 g animal) or as multiple doses (MD, twice a week for 3 weeks) of 60 μg. Anti-tumour efficacy observed in response to the two schedules was compared to that of animals “treated” with saline (MD, twice a week for 3 weeks). The antibodies were dosed intraperitoneal (IP) as 1 mg/ml in 0.9% saline. Animal weight and tumour volume were measured 3 times a week from the day of tumour cell injection. Tumour volume was calculated by use of the modified ellipsoid formula ½(Length×Width²). Mice were kept on study until their tumour reached an average diameter of 12 mm or, rarely, when incidence of tumour ulceration was observed (welfare).

TABLE E23-1
Treatment groups.

| | Number | |
|---|---|---|
| | of | |
| Group | animals | Treatment regimen (IP injection) |
| 1 | 10 | Saline (multiple dose from day 8, twice a week for 3 |
| | | weeks) |
| 2 | 10 | STIM003 mIgG2A (multiple dose from day 8, |
| | | twice a week for 3 weeks) |
| 3 | 10 | STIM003 mIgG2A (Single dose on day 8) |

### 1.11.2. Results

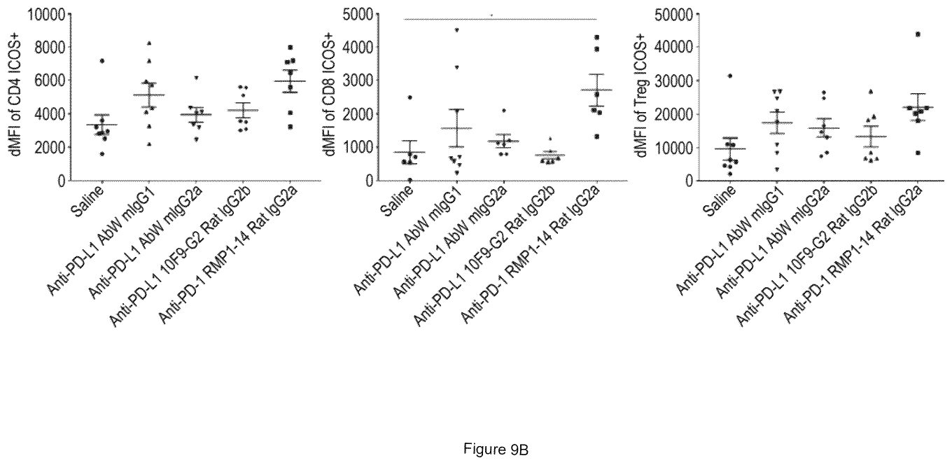

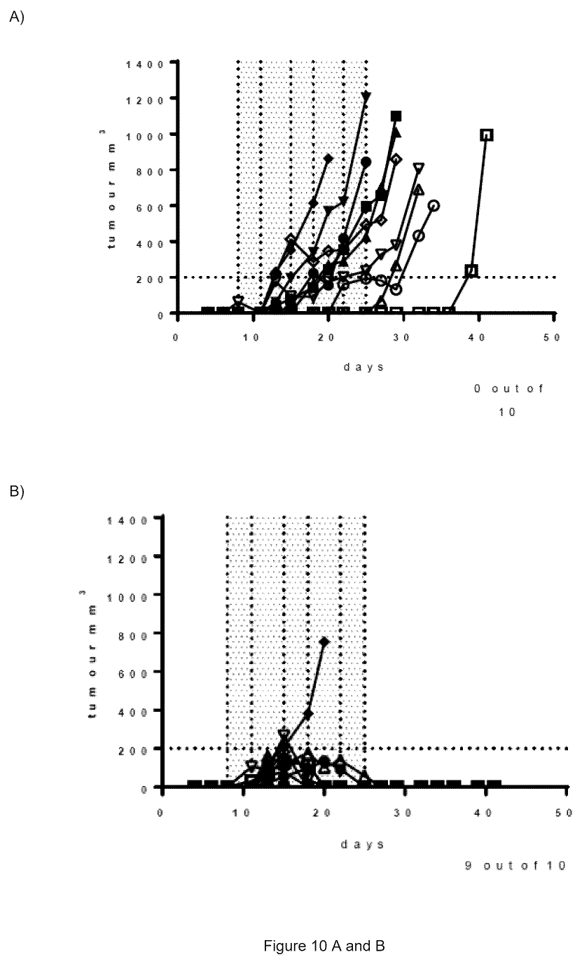

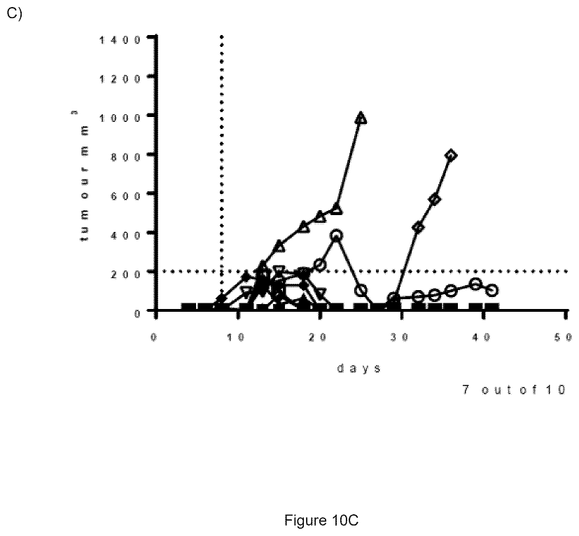

Both multiple and single dose of STIM003 mIgG2a resulted in strong and significant monotherapy anti-tumour efficacy as shown by the number of animals with no signs of tumour growth at endpoint (Day 41). SD resulted in 7 our 10 animals cured from the disease whereas the multiple dose cured 9 out of 10 animals injected with A20 B cell lymphoblast. All animals in the saline treated group had to be removed from the study by day 40 due to tumour size. See FIG. 10.

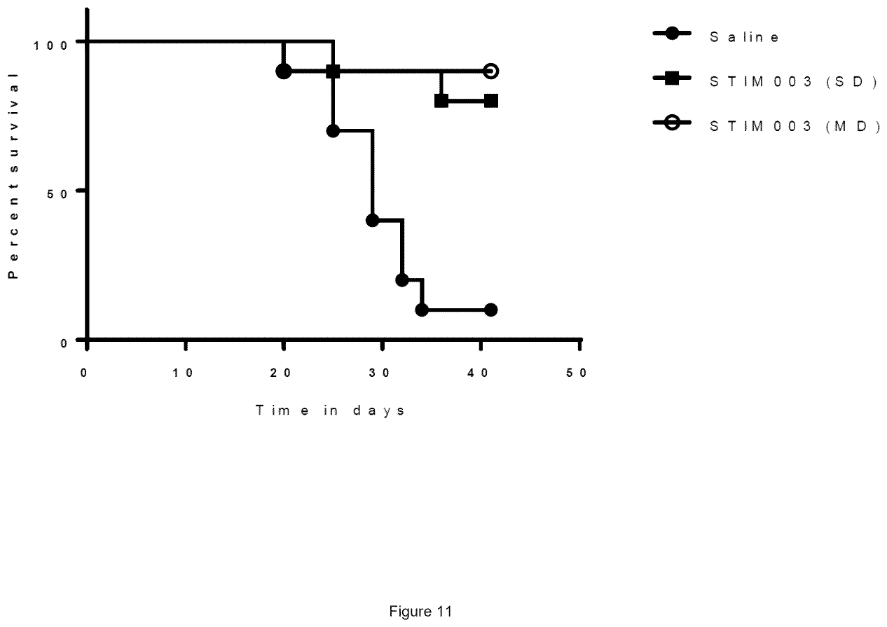

Humane endpoint survival statistics were calculated from the Kaplan-Meier curves (FIG. 11) using GraphPad Prism V7.0. This approach was used to determine if the treatments were associated with improved survival. The Hazard Ratio (Mantel-Haenszel) values and their associated P values (Log-Rank Mantel-Cox) are shown in the table below.

TABLE E23-1
Hazard Ratio (Mantel-Haenszel) values and their associated P values
(Log-Rank Mantel-Cox) corresponding to FIG. 11 Kaplan-Meier curves.

| Hazard Ratio (Mantel-Haenszel) | MD vs Saline | SD vs Saline | MD vs SD |
|---|---|---|---|
| Ratio (and its reciprocal) | 0.09995 | 0.1076 | 0.5314 |
| 95% CI of ratio | 0.02604 to 0.3837 | 0.02856 to 0.4052 | 0.05522 to 5.115 |
| P Value | 0.0008 | 0.001 | 0.5842 |

### 1.12. Example 5: Time and Dose Dependent Effects of Anti-ICOS Antibody in CT-26 Tumour Bearing Animals

This Example presents the results of a pharmacodynamic study evaluating the effects of anti-ICOS antibody on immune cells in mice bearing CT-26 tumours. T and B cell subtypes from different tissues were analysed by FACS after a single dose of STIM003 mIgG2a.

### 1.12.1. Methods

CT-26 tumour bearing animals were dosed i.p. with either saline or STIM003 at 200 μg, 60 μg or 6 μg on day 12 post tumour cell implantation. Tumour tissues, blood, tumour draining lymph node (TDLN) and spleen were harvested on day 1, 2, 3, 4, and day 8 post treatment. The tumours were dissociated to make single cell suspension using mouse tumour dissociation kit (Miltenyi Biotec). Spleen tissue was dissociated using gentle MACS dissociation, red blood cells were lysed using RBC lysis buffer. Tumour draining lymph nodes were mechanically disaggregated to make single cells suspensions. The resulting cell suspensions were clarified through either 70 μM or 40 μM filters depending on the tissue, cells were then washed twice in RMPI complete media and finally resuspended in ice cold FACS buffer. Total blood was collected into plasma tubes and red blood cells were lysed using RBC lysis buffer, cells were washed twice in RMPI complete media and finally resuspended in ice cold FACS buffer. The single cell suspension from all the tissues were distributed into 96 deep well plates for FACS analysis. Cells were stained with Live Dead Fixable Yellow viability dye (Life technologies). The cell suspensions were incubated with anti-CD16/CD32 mAb (eBioscience) and stained with FACS antibodies specific for CD3 (17A2), CD45 (30-F11), CD4 (RM4-5), CD8 (53-6.7), CD25 (PC61.5), ICOSL (HK5.3), B220 (RA3-6B2), Ki-67 (SolA15), CD107a (eBiolD4B), IFN-7 (XMG1.2), TNF-α (MP6-XT22), Foxp3 (FJK-16s) and ICOS (7E.17G9) all obtained from eBioscience Ltd. For cytokine readout by FACS, single cells suspensions from the tumours were plated in 24 well plate for 4 hours in the presence of Brefeldin-A. For the intracellular staining, samples were fixed, permeabilised, and stained with specific antibodies. The samples were finally resuspended in PBS and data acquired on the Attune flow cytometer (Invitrogen) and analysed using FlowJo V10 software (Treestar).

Results are presented and discussed below.

### 1.12.2. ICOS Expression is High on Intra-Tumoral T-Regs in the CT26 Model

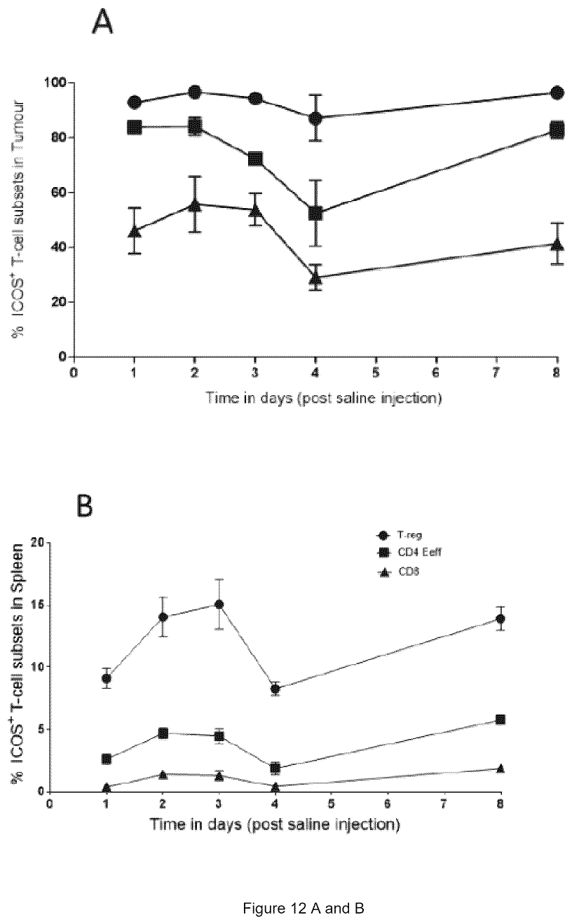

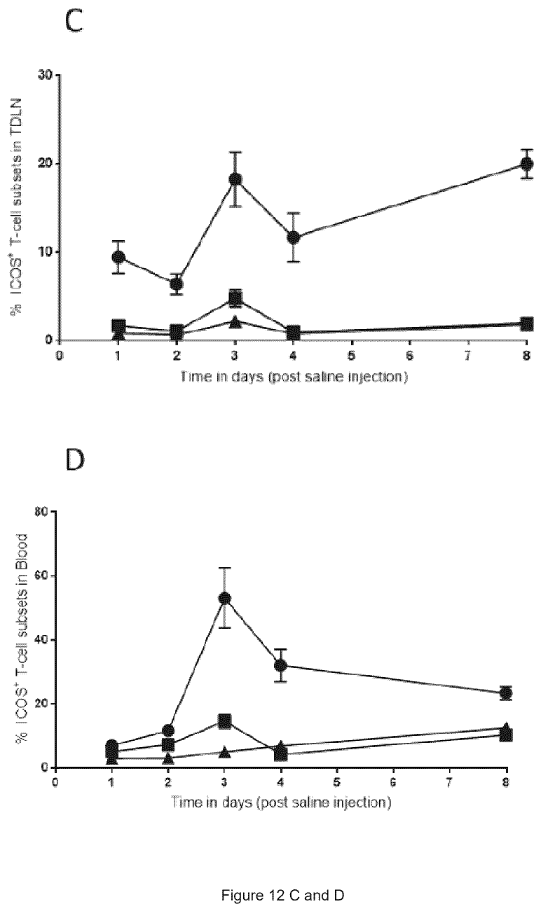

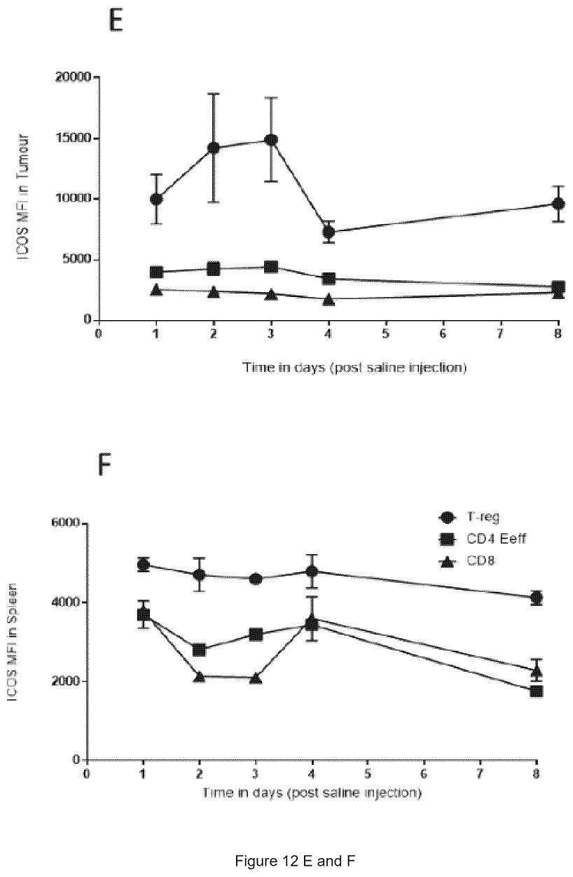

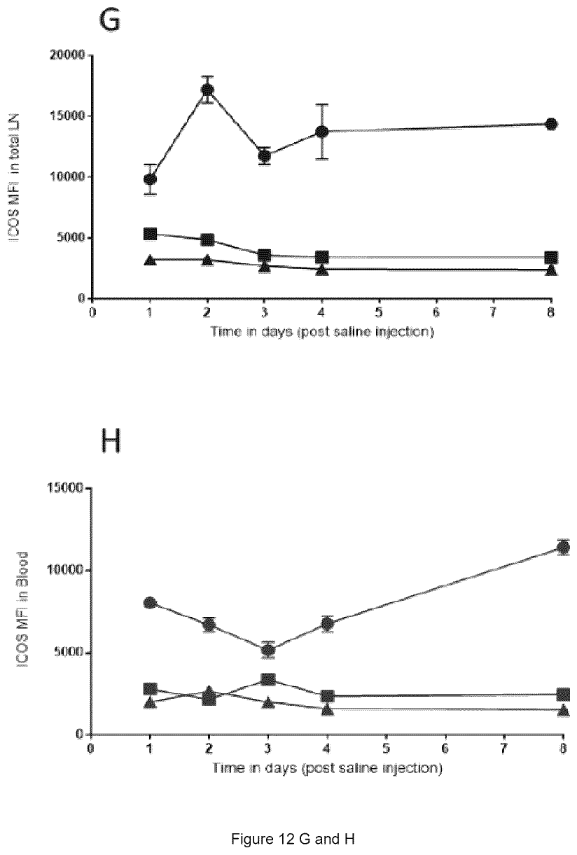

When the percentage of tumour infiltrating lymphocytes (TILs) expressing ICOS was compared to the percentage of immune cells in the spleen, blood, and TDLN, we demonstrated that more immune cells in the microenvironment of CT-26 tumours expressed ICOS vs other tissues. More importantly, the percentage of ICOS positive T-reg cells in all the tissues and at all the time points was higher than the percentage of CD4 or CD8 effector T cells positive for ICOS. Importantly, the dMFI (relative expression) for ICOS also followed the similar ranking in expression with intra-tumoural T-reg being highly positive for ICOS expression vs other TILs subtypes. Interestingly, there was no striking change in the percentage of ICOS⁺ TILs within the time frame of this experiment. Similar results were also seen in spleen and TDLN. On the other hand, in the blood, ICOS expression is relatively stable on T effector cells but increased on T-regs during the course of the experiment. Altogether the data demonstrated that more cells expressed ICOS in the tumour microenvironment and these positive cells also expressed more ICOS molecules on their surface. More importantly, T regs in TILs are highly positive for ICOS. See FIG. 12.

### 1.12.3. Strong Depletion of Intra-Tumoural T-Reg Cells in Response to STIM003 Administration

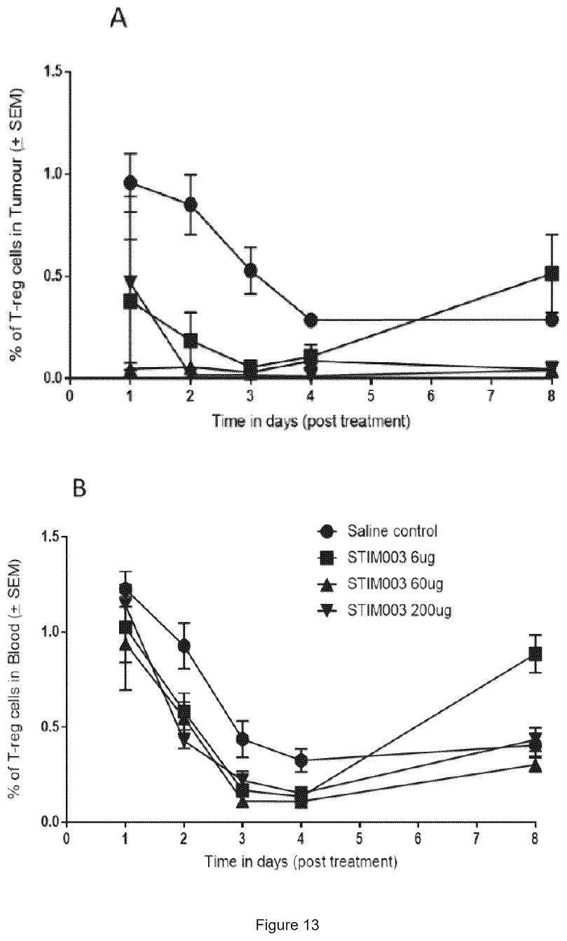

In response to the STIM003 mIgG2a antibody, there was strong and rapid depletion of T-reg cells (CD4+CD25+Foxp3) in TME. As T-regs have high ICOS expression compared with the other T cells subsets, it is expected that an anti-ICOS antibody with effector function would preferentially deplete these cells. At the lower dose of STIM003 (6 μg corresponding to a 0.3 mg/kg for a 20 g animal) there was a continuous depletion of T-reg and by day 3 most of the T-reg were depleted from TME. Interestingly, by day 8, T-reg cells repopulate the TME then reach a level slightly above that observed in the saline treated animals. The repopulation of T-reg cells at lower dose can be attributed to the increase in the proliferating CD4 T cells in TME as evidenced by an observed increase in Ki-67+CD4 T-cells. At a dose higher than 6 μg there was a long-term depletion of T-reg cells in TME as shown by full T Reg depletion until the last time point analysed in this study (day 8). Whereas in the blood there was a transient depletion of T-reg cells at all doses. Importantly, by day 8, all the treated animals had similar (or higher for the 6 μg dose) level of T-reg cells in the blood when compared to the saline treated animals. Data are shown in FIG. 13. Notably, and similarly to data previously published for depleting CTLA-4 antibodies, there was no significant change in the percentage of T-reg cells in the spleen or TDLN tissues, suggesting that T-reg cells may be protected from depletion in these organs.

In summary, strong depletion of T-reg cells in TME was achieved in CT-26 model at a dose as low as 6 μg per animal. However, a dose of 60 μg resulted in long term depletion up to 8 days post STIM003 mIgG2a injection. This was not improved by using higher dose (200 μg).

1.12.4. STIM003 mIgG2a Increased CD8:T Reg and CD4:T Reg Ratios

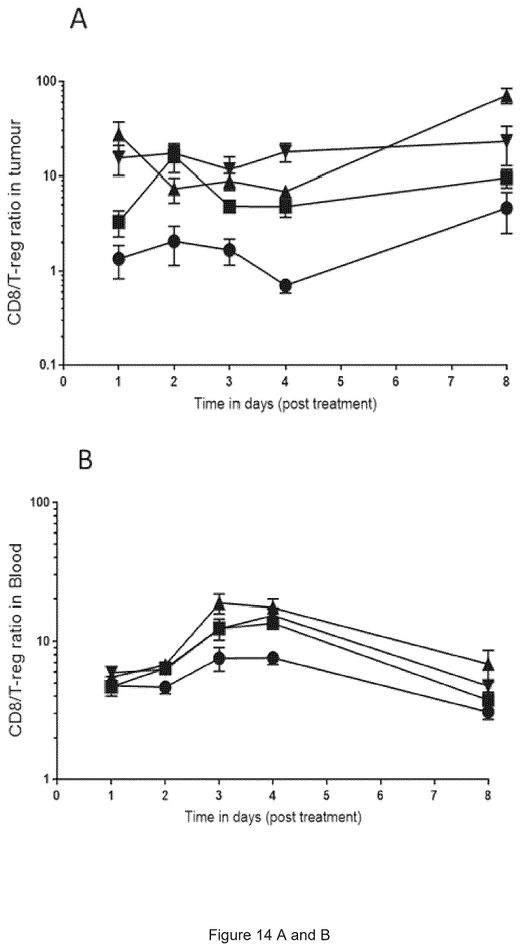

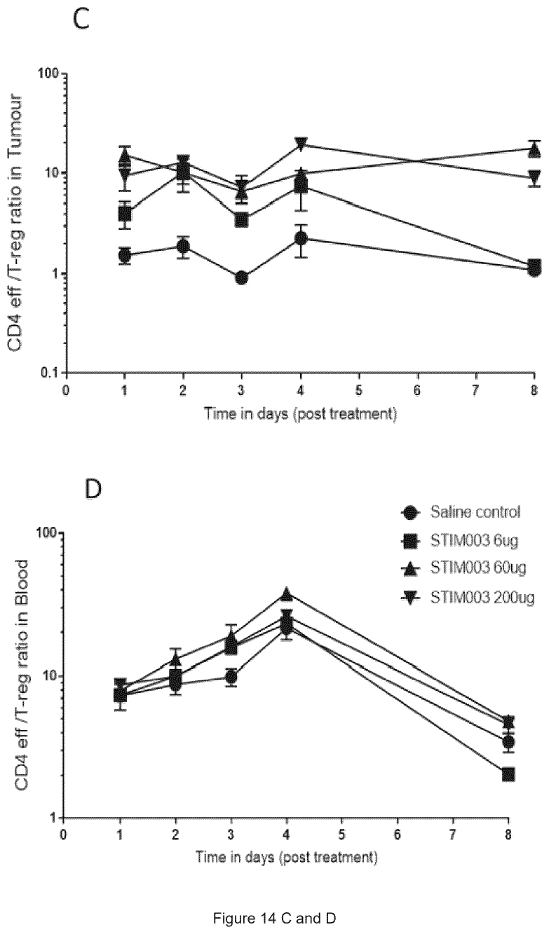

Effects of STIM003 on T-eff:T-reg ratios are shown in FIG. 14.

STIM003 mIgG2a increased the CD8:T-reg ratio as well as the CD4 eff:T-reg ratio. Although all the treatment doses were associated with an increase in T-eff to T-reg ratio, the intermediate dose of 60 μg (the equivalent of 3 mg/kg for a 20 g animals) was associated with the highest ratio by day 8 post treatment.

Interestingly, at the 6 μg dose, the ratios were high until day 4 but by day 8 post treatment they were matching that of the saline treated animals. This can be explained by the repopulation of TRegs observed for this dose by day 8 post treatment. On the other hand, at a dose of 60 or 200 μg, the Teff to T-reg ratios remained high at all time points. This is explained by a long term depletion of Tregs at these doses. Notably, at higher dose (200 μg), despite the long term Treg depletion there was only a moderate improvement in the ratio by day 8. This can be explained by some depletion of ICOS^{INT} effector cells at high concentration of STIM003.

Altogether, the data demonstrated TReg depletion and increased Effector:T reg ratio at all doses tested. However, a dose of 60 μg (˜3 mg/kg) achieved both a long-term depletion of T-reg, as well as the highest T-eff to T-reg ratios which would be associated with the most favourable immune context to initiate an anti-tumour immune response. Interestingly a similar pattern was observed in the blood, with the intermediate dose of 60 μg associated with the highest T-eff to T-reg ratio. Importantly, in the blood, improvement of the ratio was observed at an earlier time point (between day 3 and day 4).

### 1.12.5. Activation of Effector Cells in Response to STIM003

Surface expression of CD107a on the tumour infiltrating T effector cells was previously identified as a reliable marker for cells that have been activated and exert cytotoxic activity [40]. In the present study employed this marker to confirm that STIM003, in addition to depleting T-regs, can stimulate the cytotoxic activity of effector T cells in the TME. Interestingly, on day 8 post treatment, there was an increase in surface expression of CD107a on both the CD4 and CD8 effector T cell compartments at all doses of STIM003. Furthermore, this upregulation of CD107a expression on the surface on both CD4 and CD8 T cells appeared to plateau when animals were dosed at 60 μg as no improvement was seen at 200 μg dosing. ⁴⁰ Rubio V., et al. Ex vivo identification, isolation and analysis of tumor-cytolytic T cells. Nat Med. 2003 November; 9(11):1377-82.

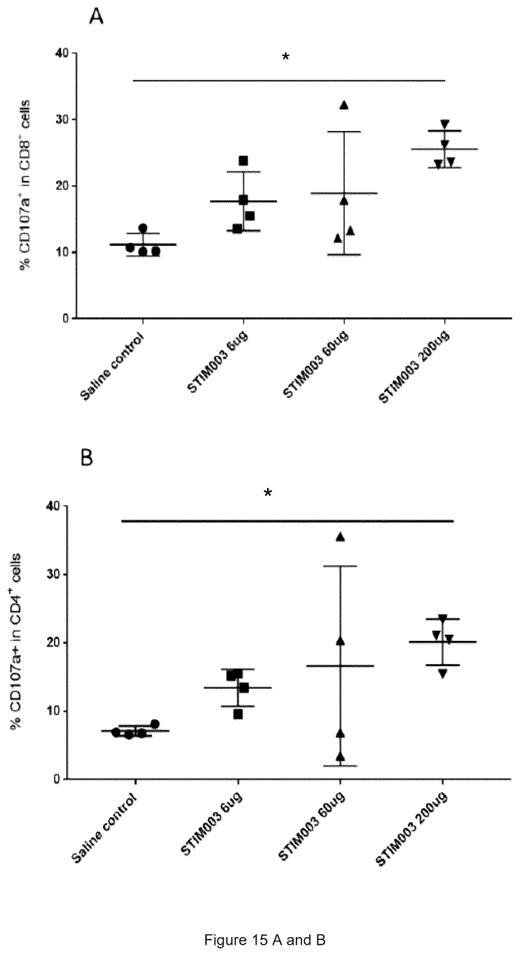

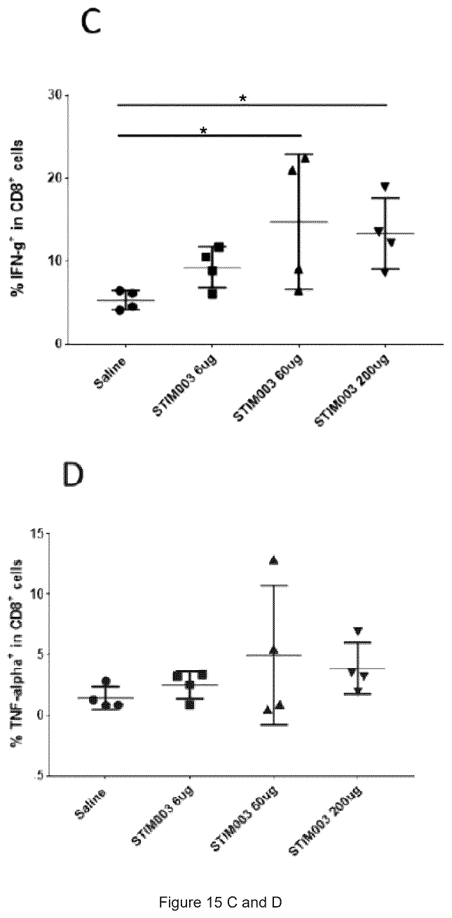

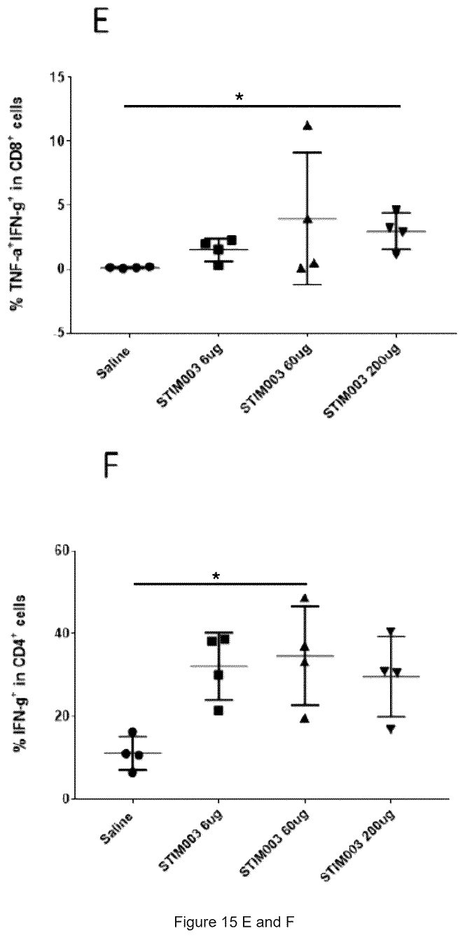

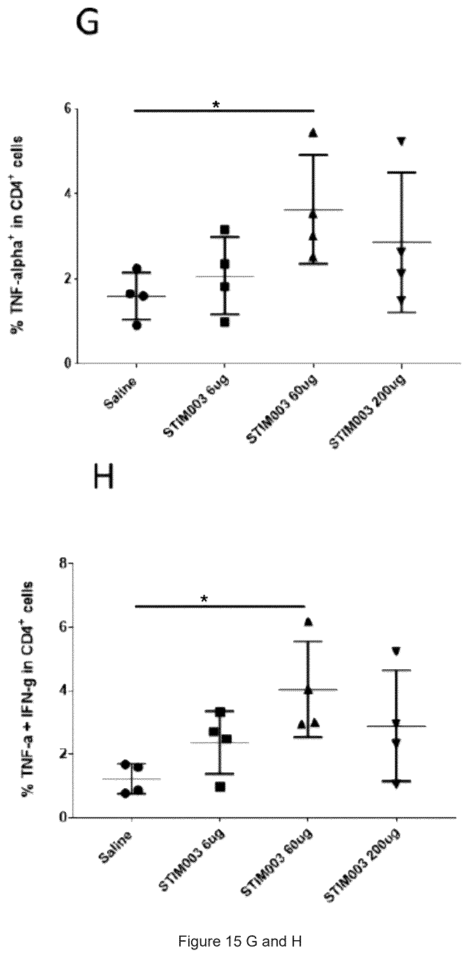

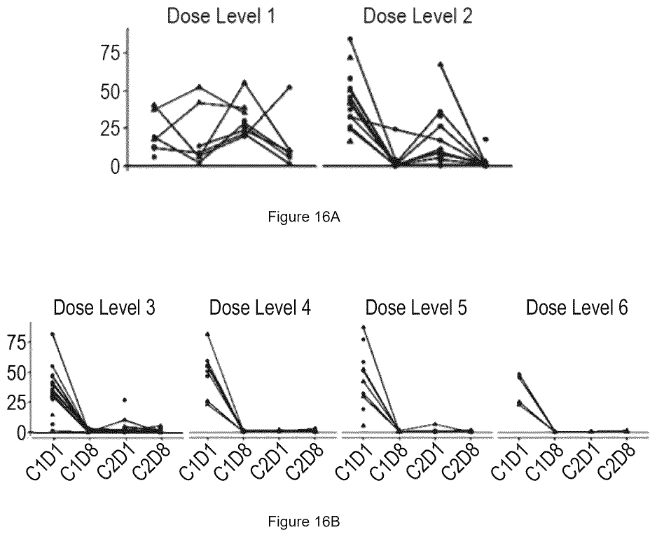

To further demonstrate activation of effector cells in the TME, the cytokine release by CD4 and CD8 TILs was analysed by FACS. As expected and consistent with the in-vitro agonism data presented in earlier Examples herein, STIM003 mIgG2a at all doses promoted pro-inflammatory cytokine IFN-γ and TNF-α production by effector CD4 and CD8 T cells. The induction of pro-inflammatory cytokine production appeared to be high at the dose of 60 μg. Indeed, 60 μg of STIM003 significantly increased cytokine production by CD4 T cells. A similar trend was seen for the proinflammatory cytokine IFN-γ and TNF-α production by effector CD8 T cells in TME. Data are shown in FIG. 15.

In summary, STIM003 at all the doses resulted in T cells activation in the TME as shown by (1) the presence of the degranulation marker CD107a on their surface and (2) by the production of Th1 cytokines (IFNγ and TNFα) by T cells. This indicates that STIM003 strongly affects the immune context in the TME and plays the dual role of depleting Treg cells and stimulate the killing activity of T effector cells.

### 1.12.6. Human Dose Estimations

Based on the pre-clinical efficacy data seen in mice, initial predictions can be made of the clinical dose appropriate for human patients, based on corresponding biological surface area (BSA) [41]. ⁴¹ Nair & Jacob., A simple practice guide for dose conversion between animals and human. J Basic Clin Pharma 2016; 7:27-31

For example, taking the anti-ICOS IgG dose in mouse to be 3 mg/kg (60 μg), and following the methods of ref [40], the corresponding dose for a human is 0.25 mg/kg.

Using the Mosteller formulae, for an individual of 60 kg and 1.70 m the BSA 1.68 m². Multiplying the dose in mg/kg by a factor of 35.7 (60/1.68) gives a fixed dose of 15 mg. For an individual of 80 kg the corresponding fixed dose would be 20 mg.

Doses may be adjusted for human therapy in clinical trials to determine safe and effective treatment regimens.

### 1.13. Example 6: Bioinformatic Analysis of Data from Tumour Samples

One target group of cancers according to the present invention is those cancers that are associated with a relatively high level of ICOS+ immunosuppressive Tregs.

To identify cancer types associated with a high content of Tregs, transcriptome data was obtained from The Cancer Genome Atlas (TCGA) public dataset and analysed for ICOS and FOXP3 expression levels. TCGA is a large-scale study that has catalogued genomic and transcriptomic data accumulated for many different types of cancers, and includes mutations, copy number variation, mRNA and miRNA gene expression, and DNA methylation along with substantial sample metadata.

Gene Set enrichment analysis (GSEA) was conducted as follows. Gene expression RNA seq data collected as part of the TCGA consortium was downloaded from the UCSC Xena Functional Genomics Browser as log 2(normalized_count+1). Non-tumour tissue samples were removed from the dataset, leaving data for 20530 genes from 9732 samples. An algorithm from [42] and its implementation in [43] that calculates enrichment scores for genes within a specified gene set was used to transpose gene level counts to gene set scores for each sample. The gene set of interest was defined as containing both ICOS and FOXP3. Samples were grouped by primary disease and the ssGSEA scores for each group were compared across the 33 primary disease groups. The disease groups that showed the highest median scores were found to be lymphoid neoplasm diffuse large b-cell lymphoma, thymoma, head and neck squamous cell carcinoma, although diffuse large b-cell lymphoma showed a multimodal distribution of scores with a subset scoring highly and the rest scoring below the group median. ⁴² D. A. Barbie, et al., “Systematic RNA interference reveals that oncogenic KRAS-driven cancers require TBK1.,” Nature, vol. 462, no. 7269, pp. 108-12, 2009⁴³ S. Hanzelmann, R. Castelo, and J. Guinney, “GSVA: gene set variation analysis for microarray and RNA-Seq data,” BMC Bioinformatics, vol. 14, no. 1, p. 7, 2013

In rank order of highest to lowest ssGSEA score for ICOS and FOXP3 expression, the top 15 cancer types were:

| | |
|---|---|
| DLBC (n = 48) | lymphoid neoplasm diffuse large b-cell lymphoma |
| THYM (n = 120) | thymoma |
| HNSC (n = 522) | head and neck squamous cell carcinoma |
| TGCT (n = 156) | testicular germ cell tumour |
| STAD (n = 415) | stomach adenocarcinoma |
| SKCM (n = 473) | skin cutaneous melanoma |
| CESC (n = 305) | cervical squamous cell carcinoma and |
| | endocervical adenocarcinoma |
| LUAD (n = 517) | lung adenocarcinoma |
| LAML (n = 173) | acute myeloid leukemia |
| ESCA (n = 185) | esophageal carcinoma |
| LUSC (n = 502) | lung squamous cell carcinoma |
| READ (n = 95) | rectum adenocarcinoma |
| COAD (n = 288) | colon adenocarcinoma |
| BRCA (n = 1104) | breast invasive carcinoma |
| LIHC (n = 373) | liver hepatocellular carcinoma |

In which n is the number of patient samples for that cancer type in TCGA dataset. Anti-ICOS antibodies described herein may be used for treatment of these and other cancers.

Cancers that are associated with a relatively high level of ICOS+ immunosuppressive Tregs and which further express PD-L1 may respond especially well to treatment with a combination of anti-ICOS antibody and anti-PD-L1 antibody. Appropriate treatment regiments and antibodies for this purpose have already been detailed in the foregoing description.

Using the TCGA dataset as before, enrichment scores for ICOS and FOXP3 were correlated with expression levels of PD-L1 using Spearman's rank correlation and grouped by primary disease indication. P-values were calculated for each group and a p-value of 0.05 (with Bonferroni's multiple comparison correction) was taken as statistically significant. The disease groups with the highest correlations between ICOS/FOXP3 and PD-L1 expression were:

| | |
|---|---|
| TGCT (n = 156) | testicular germ cell tumour |
| COAD (n = 288) | colon adenocarcinoma |
| READ (n = 95) | rectum adenocarcinoma |
| BLCA (n = 407) | bladder urothelial carcinoma |
| OV (n = 308) | ovarian serous cystadenocarcinoma |
| BRCA (n = 1104) | breast invasive carcinoma |
| SKCM (n = 473) | skin cutaneous melanoma |
| CESC (n = 305) | cervical squamous cell carcinoma and |
| | endocervical adenocarcinoma |
| STAD (n = 415) | stomach adenocarcinoma |
| LUAD (n = 517) | lung adenocarcinoma |

Patients may be selected for treatment following an assay determining that their cancer is associated with ICOS+ immunosuppressive Tregs and expression of PD-L1. For cancer types in which, as above, there is a high correlation score, it may suffice to determine that one of ICOS+ immunosuppressive Tregs and expression of PD-L1 is present (e.g., above a threshold value). PD-L1 immunohistochemistry assays may be used in this context.

### 1.14. Example 7: Assessment of Further Anti-ICOS Antibodies

CL-74570 and CL-61091 antibody sequences were synthesised and expressed in IgG1 format in HEK cells.

Functional characterisation of these antibodies was performed using an HTRF assay as described previously (see, e.g. Example 6 of U.S. Pat. No. 9,957,323), with modifications to adapt the assay to use of purified IgG1 rather than BCT supernatant. 5 μL of supernatant containing human IgG1 antibodies expressed from HEK cells was used in place of the BCT supernatant, and the total volume made up to 20 μl per well using HTRF buffer as before. A human IgG1 antibody was used as a negative control. Both antibodies exhibited greater than 5% effect for binding to human and mouse ICOS as calculated using Equation 1 and were therefore confirmed to test positive in this assay.

Ability of these antibodies to bind human and mouse ICOS expressed on the surface of CHO-S cells was further confirmed using a Mirrorball assay. In this assay, 5 μl supernatant containing the anti-ICOS IgG1 was transferred to each well of 384 mirrorball black plates (Corning). Binding of anti-ICOS antibodies was detected by adding 10 μl of goat anti-human 488 (Jackson Immunoresearch) diluted in assay buffer (PBS+1% BSA+0.1% Sodium Azide) at a concentration of 0.8 mg/ml to all wells.

For positive control wells, 5 μL reference antibody diluted in assay media to 2.2 μg/mL was added to the plates. For negative control wells, 5 μl of Hybrid control IgG1 diluted in assay media to 2.2 μg/mL was added to the plates. 10 μM of DRAQ5 (Thermoscientific) was added to 0.4×10⁶/ml cells resuspended in assay buffer and 5 μl was added to all wells. Plates were incubated for 2 hr at 4 degrees.

Fluorescence intensity was measured using Mirrorball plate reader (TTP Labtech), measuring Alexafluor 488 (excitation 493 nm, emission 519 nm) from a population of 500-700 single cells. Assay signal was measured as Median (FL2) Mean Intensity.

Total binding was defined using reference antibody at an assay concentration of 2.2 μg/mL. Non-specific binding was defined using Hybrid control hIgG1 at an assay concentration of 2.22 μg/mL. Both antibodies exhibited greater than 1 percent effect and were therefore confirmed to test positive in this assay.

$$Percent effect = \frac{( sample well - non - specific binding )}{( total binding - non - specific binding )} \times 100$$

Each of CL-74570 and CL-61091 also demonstrated binding to human and mouse ICOS expressed on CHO-S cells as determined by flow cytometry. FACS screening was performed using purified IgG1 rather than BCT supernatant. Both antibodies exhibited binding>10 fold above the average of geomean of the negative control binding to hICOS, mICOS and WT CHO cells.

TABLE E26-1
Functional characterisation of CL-74570 and CL-61091.

| Primary Screen | | | | | | |
|---|---|---|---|---|---|---|
| | | Mirrorball | | Secondary | | |
| HTRF (Protein) | | (ICOS CHO Cell) | | screen FACS | | |
| Human | Mouse | Human | Mouse | Human | Mouse | |
| 1:100 dil | 1:100 dil | 1:100 dil | 1:100 dil | ICOS CHO | ICOS CHO | |
| Percent | Percent | Percent | Percent | (1:10 dil) | (1:10 dil) | |
| Effect | Effect | Effect | Effect | % Binding- | % Binding- | Clone |
| [%] | [%] | [%] | [%] | APC | APC | ID |
| 94.42 | 60.86 | 107.02 | 127.03 | 122.97 | 96.41 | CL- |
| | | | | | | 74570 |
| 83.43 | 76.65 | 54.14 | 113.10 | 19.08 | 62.94 | CL- |
| | | | | | | 61091 |

### 1.15. Example 8: Clinical Trial Phase I/II Open-Label Study of KY1044

A phase I/II open-label study of KY1044, an anti-ICOS antibody with dual mechanism of action, as single agent and in combination with atezolizumab, was performed on adult patients with advanced malignancies. Participants included patients with advanced/metastatic malignancies who have had measurable disease (non-measurable disease was allowed only in Phase I) as determined by Response Evaluation Criteria in Solid Tumours version 1.1 (RECIST 1.1) and were eligible if, according to the National Comprehensive Cancer Network (NCCN) guidelines, there were no available therapies known to confer a clinical benefit for their disease, or they had exhausted all such available options.

### 1.15.1. Methods

### Study Arms:

KY1044 monotherapy phase I: KY1044 monotherapy dose escalation
KY1044 and atezolizumab phase I: KY1044 and atezolizumab combination dose escalation
KY1044 monotherapy phase II: KY1044 monotherapy
KY1044 and atezolizumab phase II: KY1044 and atezolizumab combination

Phase I: Participants with advanced/metastatic malignancies, and preferred indications (non-small cell lung cancer (NSCLC), head and neck squamous cell carcinoma (HNSCC), hepatocellular carcinoma (HCC), melanoma, cervical cancer, esophageal cancer, gastric cancer, renal cell carcinoma, pancreatic cancer, and triple negative breast cancer).

Phase II KY1044 single agent: Participants with advanced/metastatic malignancies in indications in which signs of anti-tumour activity (Complete Response (CR), Partial Response (PR) or durable stable disease (SD) with tumour shrinkage that does not qualify for PR) were seen during the dose escalation of KY1044 as single agent.

Phase II KY1044 in combination with atezolizumab: Participants with advanced/metastatic malignancies in the selected indications below, and/or indications which have shown promising activity in Phase I:
NSCLC (anti-PD-(L)1 therapy naïve and pre-treated)
Gastric (anti-PD-(L)1 therapy naïve and pre-treated)
HNSCC (anti-PD-(L)1 therapy naïve and pre-treated)
Esophageal (anti-PD-(L)1 therapy naïve and pre-treated)
Cervical (anti-PD-(L)1 therapy naïve and pre-treated)
Indications, in which signs of anti-tumor activity has been observed in Phase I with
KY1044 in combination with atezolizumab.

Patients with advanced/metastatic malignancies received escalating doses of KY1044 as a single agent or in combination with 1200 mg of anti-PD-L1 antibody, atezolizumab, by IV infusion every 3 weeks until disease progression or unacceptable toxicity. Dose escalation was guided by a modified toxicity probability interval design. The primary objective was to determine safety, tolerability, and maximum tolerated dose. Cohorts that were tolerated were later enriched with more subjects. Adverse events (AEs) were classified according to Common Terminology Criteria for Adverse Events version 5 (CTCAE v5) and efficacy measures performed according to Response Evaluation Criteria in Solid Tumours version 1.1 (RECIST v1.1) every 8 weeks for the first 16 weeks and then every 12 weeks.

### 1.15.2. Patient Inclusion Criteria:

Participants must have met all of the following additional inclusion criteria:
1. Prior therapy with anti-PD-(L)1 and/or anti-PD-L1 inhibitors was allowed provided any toxicity attributed to prior anti-PD-(L)1 and/or anti-PD-L1-directed therapy did not lead to discontinuation of therapy;
2. Eastern Cooperative Oncology Group (ECOG) Performance Status 0-1;
3. Life expectancy longer than 12 weeks; and
4. A site of disease amenable to biopsy and be a candidate for tumour biopsy according to the treating institution's guidelines.

### 1.15.3. Patient Exclusion Criteria:

Patients must not have had any of the following exclusion criteria:
1. Presence of symptomatic central nervous system (CNS) metastases, or CNS metastases that require local CNS-directed therapy, or increasing doses of corticosteroids within the prior 2 weeks of first dose of study treatment;
2. History of severe hypersensitivity reactions to other monoclonal antibodies and/or their excipients;
3. Known presence of neutralizing anti-atezolizumab antibodies (for patients previously treated with atezolizumab);
4. Having out of range laboratory values: creatinine, bilirubin, alanine aminotransferase (ALT), aspartate aminotransferase (AST), absolute neutrophil count (ANC), platelet count, hemoglobin;
5. Impaired cardiac function or clinically significant cardiac disease;
6. Known human immunodeficiency virus (HIV), active hepatitis B virus (HBV) or active hepatitis C virus (HCV) infection;
7. Malignant disease, other than that being treated in this study;
8. Any medical condition that would, in the Investigator's judgment, prevent participation in the clinical study due to safety concerns, compliance with clinical study procedures or interpretation of study results;
9. Active autoimmune disease or a documented history of autoimmune disease;
10. Participants previously exposed to anti-PD-(L)1 treatment who are not adequately treated for skin rash or had no replacement therapy for endocrinopathies should be excluded;
11. Participants with a history of drug-induced pneumonitis or current pneumonitis;
12. Systemic steroid therapy or any immunosuppressive therapy. Topical, inhaled, nasal, and ophthalmic steroids are not prohibited;
13. Use of life attenuated vaccines against infectious diseases within 4 weeks of the first dose of study treatment;
14. Anti-CTLA4, anti-PD-(L)1 treatment within 4 weeks of the first dose of study treatment;
15. Pre-treatment with anti-CTLA4 antibodies in combination with any other antibody or drug specifically targeting T-cell co-stimulation or checkpoint pathway;
16. Presence of Common Terminology Criteria for Adverse Events version 5 (CTCAE v5)≥Grade 2 toxicity (except alopecia, peripheral neuropathy and ototoxicity, which are excluded if CTCAE v5≥Grade 3) due to prior cancer therapy;
17. Radiotherapy within 2 weeks of the first dose of study treatment, except for palliative radiotherapy to a limited field, such as for the treatment of bone pain or a focally painful tumour mass. To allow evaluation for response to treatment, participants enrolled in the Phase II part must have remaining measurable disease that has not been irradiated; and
18. Pregnant or lactating women.

### 1.15.4. Dosing

KY1044 was administered at a dose of 0.8 mg, 2.4 mg, 8 mg, 24 mg, 80 mg, or 240 mg, every three weeks, as a single agent or in combination with 1200 mg of atezolizumab.

### 1.15.5. Interim Results

103 patients were enrolled in the study (38 patients as monotherapy in 6 cohorts at doses ranging from 0.8 to 240 mg and 65 in combination with atezolizumab in 5 cohorts at doses 0.8-80 mg). 63% and 55% of patients received≥4 prior anti-cancer therapies in the single agent and combination cohorts, respectively.

All cohorts were completed without dose limiting toxicities (DLTs) during the first 21 days of treatment. In the KY1044 single agent cohorts, 47.4% of patients experienced treatment-related AEs (TRAEs), all were Grades 1 or 2. In the combination cohorts, TRAEs were observed in 58% of patients. Most of the TRAEs were Grade 1 or 2 apart from 8 TRAEs that were ≥Grade 3 occurring in <8% of patients. Infusion-related reactions, pyrexia and lymphopenia were the most commonly occurring TRAEs in ≥10% of patients. TRAE leading to dose interruptions occurred in 1 patient in the single agent cohort and in 4 patients in the combination cohort. Only 1 patient discontinued treatment due to myositis that was considered related to the combination.

Preliminary KY1044 data from 69 patients agreed with the pharmacokinetic (PK) model predictions.

### 1.15.6. Conclusion:

These results indicate that KY1044 was well tolerated as a single agent and in combination with atezolizumab.

### 1.16. Example 9: Clinical Trial Preliminary Pharmacodynamic Markers from Phase I/II Multicenter Trial

Longitudinal blood samples were used to correlate KY1044 target engagement levels with pharmacodynamic (PD) properties in the circulation.

### 1.16.1. Methods:

Phase I/II subjects, described in Example 8, were enrolled in dose escalation and enrichment cohorts to evaluate the effect of KY1044 as monotherapy (0.8-240 mg) every three weeks and in combination (0.8-80 mg) with atezolizumab (1200 mg) every three weeks. Peripheral blood mononuclear cells (PBMCs), plasma, and tumour biopsies were collected over the first 3 cycles to confirm target engagement and KY1044 method of action (MoA). The sample analysis included: circulating T cell receptor occupancy by chip-cytometry; PBMC and tumour sample pre- and post-treatment transcriptomic analysis; and the assessment of circulating cytokines (e.g., GM-CSF and TNFα).

### 1.16.2. Interim Results:

As assessed in PBMCs, full/prolonged ICOS target engagement on T cells was confirmed in subjects that received higher flat doses of 8 to 240 mg of KY1044, while partial/transient saturation was observed in subjects that received lower flat doses (0.8-2.4 mg). ICOS target engagement was quantified as the percentage occupancy on CD4 memory cells as measured by chip cytometry from patient blood plasma samples. The target engagement was not affected by atezolizumab. FIGS. 16A and 16B show percentage occupancy on CD4 memory for patients receiving different dose levels.

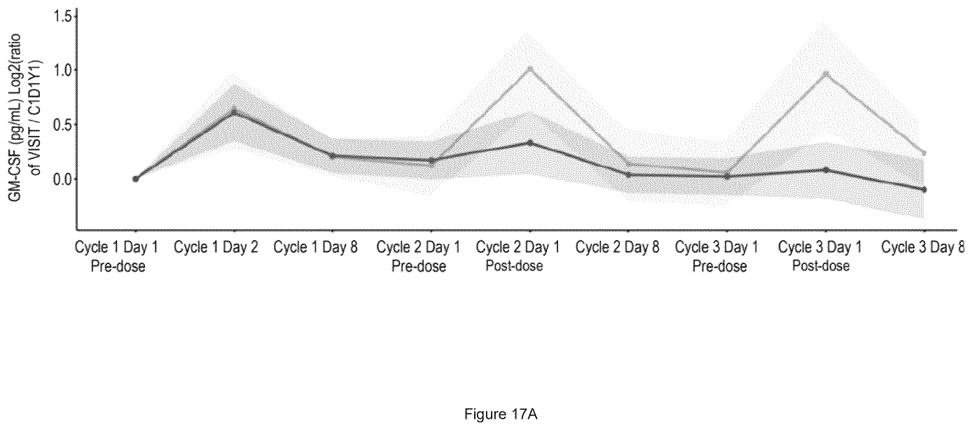

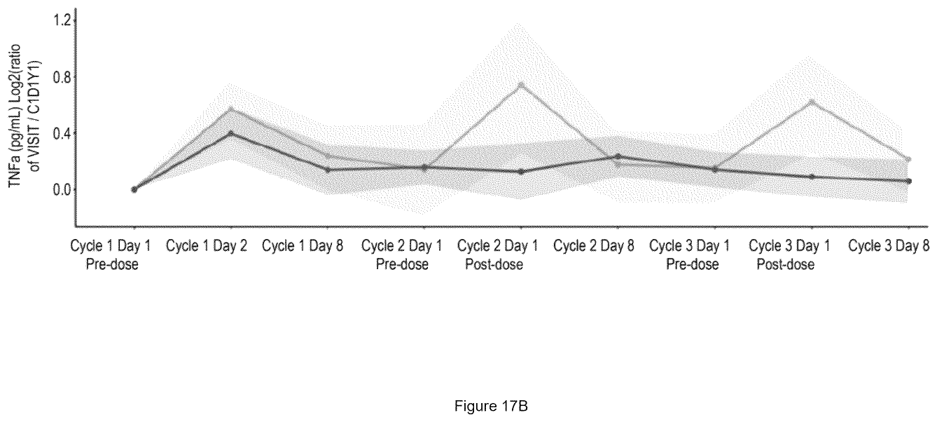

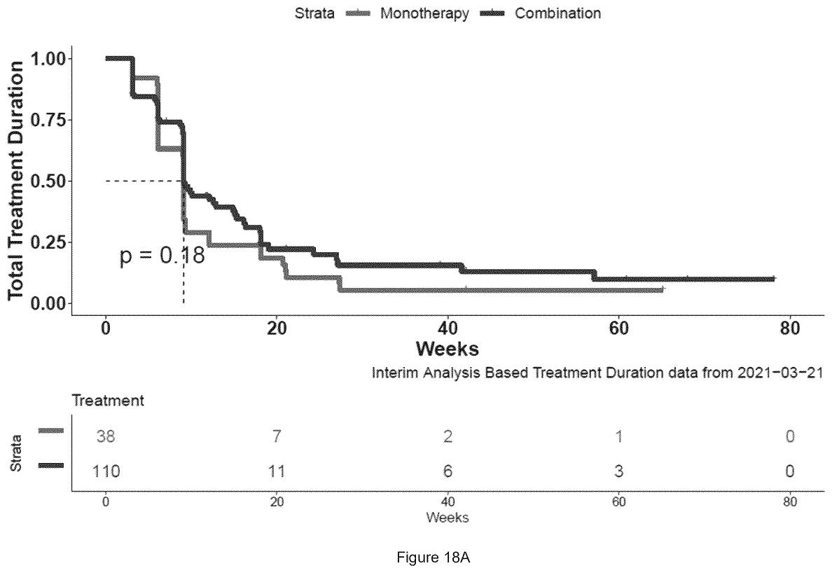

KY1044-dependent agonism was indirectly assessed by measuring circulating cytokine levels. GM-CSF and TNFα levels were assessed over the first 3 cycles and compared to values at baseline. A post-dosing transient induction of GM-CSF was evident in subjects dosed with 0.8 mg and 2.4 mg KY1044, whereas minimal induction was observed at a dose of 8 mg and higher. See FIG. 17A. A post-dosing transient induction of TNFα was also evident in subjects dosed with KY1044 at the 0.8 and 2.4 mg dose, whereas minimal induction was observed at dose of 8 mg and higher. See FIG. 17B. No association was observed between treatment and IFNγ levels.

### 1.16.3. Conclusion:

Lower doses of KY1044 (0.8 mg and 2.4 mg), which resulted in partial receptor occupancy, induced a stronger GM-SCF and TNFα signal after treatment. Dosing KY1044 in an amount that achieves less than complete receptor occupancy may therefore be advantageous insofar as it generates a pulsing cytokine response, with higher post-dosing peaks of cytokine levels on repeat administration of the lower dose levels as compared with higher dose levels.

### 1.17. Example 10: Longitudinal Pharmacodynamic Data Confirms Expected KY1044 Method of Action

Longitudinal samples were used to correlate KY1044 target engagement levels with pharmacodynamic (PD) properties (e.g., dual method of action) in the tumour microenvironment (TME).

### 1.17.1. Methods

Phase I/II subjects, described in Example 8, were enrolled in dose escalation and enrichment cohorts to evaluate the effect of KY1044 as monotherapy (0.8-240 mg) every three weeks and in combination (0.8-80 mg) with atezolizumab (1200 mg) every three weeks. Peripheral blood mononuclear cells (PBMCs), plasma, and tumour biopsies were collected over the first 3 cycles to confirm target engagement and KY1044 method of action (MoA). The sample analysis included: immunohistochemistry (IHC) of tumour samples (ICOS, FOXP3 and CD8) and circulating T cell immunoprofiling.

### 1.17.2. Interim Results:

The immune cell profiling showed changes in some populations, but there was no significant depletion of peripheral ICOS+ cells. In contrast, pre- and post-treatment IHC analysis of ICOS+/FOXP3+ cells in tumour biopsies confirmed a KY1044-dose dependent reduction of ICOS+ Tregs and maintenance of CD8+ T cells in the TME, with the highest intratumoral ICOS+ Treg depletion observed with doses of 8 mg and above. KY1044 reduced ICOS+ Tregs and improved the ratio of CD8 to ICOS+ Tregs at all tested doses in the TME, plateauing from subjects receiving a KY1044 dose of 8 mg or higher. These results indicate that KY1044 directed agonism is most evident at lower doses (0.8 mg and 2.4 mg), which are the doses that achieved partial ICOS receptor occupancy.

### 1.17.3. Conclusion:

Longitudinal PD data confirmed the KY1044 method of action, namely ICOS+ Treg depletion and increase CD8+/ICOS+ Treg ratio in the TME as well as T cell co-stimulation. These results, together with those reported in Example 9, support a dual method-of-action of KY1044. Without being bound by theory, lower doses of KY1044 (e.g., <8 mg, e.g., 2.4 mg or 0.8 mg) may stimulate an increase in cytokine response (increase in pro-inflammatory cytokines GM-CSF and TNFα) and simultaneously mediate an intra-tumoural reduction in ICOS+ Tregs and improve the ratio of CD8 to ICOS+ Tregs.

### 1.18. Example 11: Interim Results from Phase I/II Trial: Partial and Complete Responses to Combination Therapy

### 1.18.1. Methods

Methods used are recited in Example 8.

### 1.18.2. Patient Inclusion Criteria:

Patients were enrolled as described in Example 8.

### 1.18.3. Interim Results:

Interim results from the Phase I/II show signs of anti-tumour activity. Partial responses (PR) or complete responses (CR) were observed in the trial. Objective responses documented include:

| | |
|---|---|
| CR in triple negative | 2.4 mg KY1044 + 1200 mg atezo |
| breast cancer (TNBC) | |
| PR in TNBC | 2.4 mg KY1044 + 1200 mg atezo |
| PR in head and neck | 8 mg KY1044 + 1200 mg atezo |
| squamous cell carcinoma | |
| PR in penile cancer | 24 mg KY1044 + 1200 mg atezo |
| PR in pancreatic cancer | 0.8 mg KY1044 + 1200 mg atezo |

The KY1044 dose administered to these patients (with 1200 mg atezolizumab) is indicated.

Complete response was defined according to RECIST 1.1 and irRESIST as follows: Complete response (CR): disappearance of all target lesions. Any pathological lymph nodes (whether target or nontarget) must have reduction in short axis to <10 mm. CR: disappearance of all nontarget lesions and (if applicable) normalization of tumor marker level). All lymph nodes must be non-pathological in size (<10 mm short axis).

Partial response was defined according to RECIST 1.1 and iRESIST as follows: Partial response (PR): at least a 30% decrease in the sum of diameters of target lesions, taking as reference the baseline sum of diameters. Non-CR/Non-PD: persistence of one or more nontarget lesion(s) and/or (if applicable) maintenance of tumor marker level above the normal limits.

### 1.18.4. Conclusion:

The anti-ICOS antibody KY1044 promotes the efficacy of anti-PD-L1 antibody therapy.

### 1.19. Example 12: Interim Results from Phase I/II Trial: Treatment Duration

### 1.19.1. Methods

Methods used are recited in Example 8. Briefly, patients with advanced/metastatic malignancies received doses of KY1044 as a single agent or in combination with 1200 mg of the anti-PD-L1 antibody atezolizumab, by IV infusion every 3 weeks until disease progression or unacceptable toxicity.

### 1.19.2. Patient Inclusion Criteria:

Patients were enrolled as described in Example 8.

### 1.19.3. Interim Results:

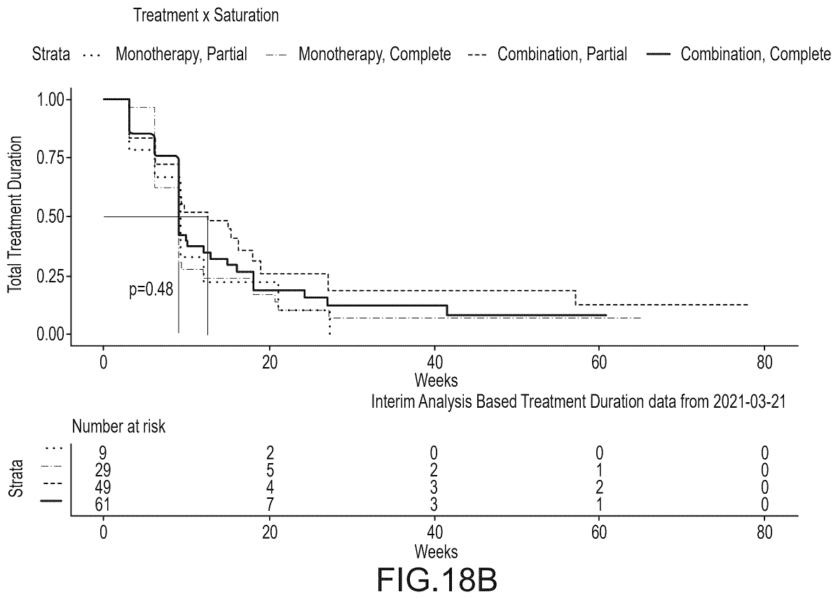

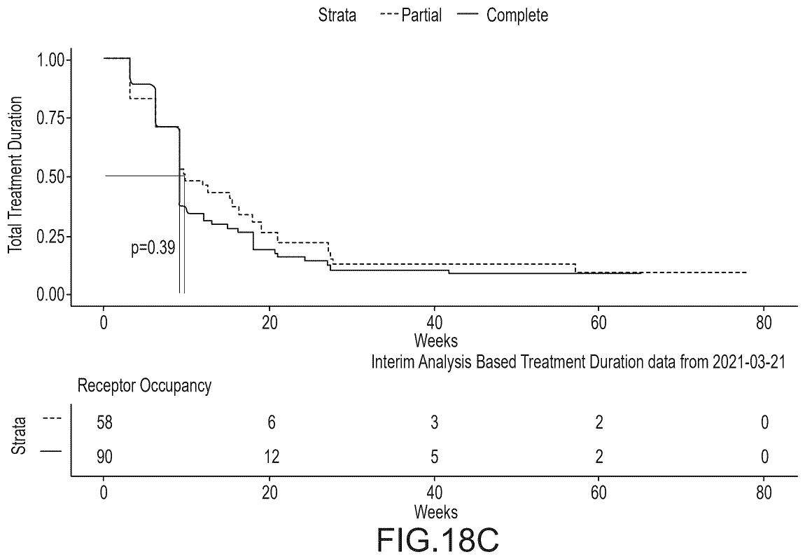

Median duration of treatment for all enrolled patients was 9 weeks. Treatment duration≥16 weeks was observed in 24% (9/38) and 27% (17/64) patients in the single agent and combination cohorts, respectively. Further data on treatment duration for monotherapy and combination therapy are provided in FIG. 18A. For example, FIG. 18A shows that a treatment duration of ≥20 weeks was observed in 18% (7/38) of patients treated with KY1044 as a single agent and in 10% (11/110) of patients treated with combination therapy.

In FIG. 18B, the data in FIG. 18A are further stratified according to partial or complete saturation (receptor occupancy), which were obtained by lower (0.8 or 2.4 mg) or higher (≥8 mg) doses of KY1044, respectively. A treatment duration of ≥20 weeks was observed in 22% (2/9) of patients that received a lower dose (0.8 mg or 2.4 mg) of KY1044 as a single agent, which resulted in partial receptor occupancy. A treatment duration of ≥20 weeks was observed in 17% (5/29) of patients that received a higher dose (≥8 mg) of KY1044 as a single agent. Treatment duration of ≥20 weeks was observed in 8% (4/49) of patients that received a lower dose (0.8 mg or 2.4 mg) of KY1044 in combination with atezolizumab (1200 mg). Treatment duration of ≥20 weeks was observed in 11% (7/61) of patients that received a higher dose (≥8 mg) of KY1044 in combination with atezolizumab (1200 mg). See FIG. 18B. Further, a treatment duration of ≥20 weeks was observed in 10% (6/58) of patients that received KY1044 at a dose that resulted in partial receptor occupancy (0.8 mg or 2.4 mg KY1044). Treatment duration of 20 weeks was observed in 13% (12/90) of patients that received KY1044 at a dose that resulted in complete receptor occupancy (≥8 mg). See FIG. 18C.

### 1.19.4. Conclusion:

These data support the surprising efficacy of lower doses (e.g., 0.8 mg, 2.4 mg) of the anti-ICOS antibody KY1044, especially in combination with an anti-PD-L1 antibody therapy.

### 1.20. Example 13: KY1044 in Combination with Atezolizumab in HNSCC Patients

### 1.20.1. Methods

In this stage of the study, Phase 2 cohorts (2 cohorts, PD-L1 naïve and pretreated) are being initiated in pts with head and neck squamous cell carcinoma (HNSCC). Approximately 40 pts will be enrolled in each cohort. Methods used are recited in Example 8. Efficacy measures will be performed as per Response Evaluation Criteria in Solid Tumours version 1.1 (RECIST 1.1) every 8 weeks for the first 16 weeks and then every 12 weeks, while adverse events (AEs) will be classified according to Common Terminology Criteria for Adverse Events version 5 (CTCAE v5).

### 1.20.2. Inclusion/Exclusion Criteria

Key inclusion criteria: anti-PD-L1 therapy naïve and pre-treated, 1-2 prior lines of systemic therapy for advanced disease, histologically documented advanced/metastatic malignancies, measurable disease by RECIST 1.1, site of disease amenable to biopsy.

Key exclusion criteria: CNS metastases, active autoimmune disease, significant heart disease and/or QT prolongation, steroid therapy, or any immunosuppressive therapy.

### 1.20.3. Interim Results:

KY1044 was well tolerated and showed initial signs of activity for HNSCC treatment. In the Phase 1 stage of the study, a 59-year-old male patient with HPV+HNSCC who had progressed on 5 prior lines of therapy (including nivolumab), experienced a partial response (42% tumor shrinkage), which was still holding as of cycle 26 day 1 (C26D1), and on treatment for >20 months (as of Feb. 10, 2022).

### 1.20.4. Conclusion:

The strong expression of ICOS on intratumoral Tregs in head and neck cancer (Sainson R et al. Cancer Immunol Res. 8, 2020:1568-82) as well as the promising clinical activity in one HNSCC patient (Patel M R et al. J Clinical Oncology 39, 2021 (suppl 15; abstract 2624) with a treatment duration of >20 months suggest HNSCC to be a favorable indication for KY1044 (SAR445256).

### 1.21. Sequences

1.21.1.1. Antibody STIM001
VH domain nucleotide sequence:
SEQ ID NO: 367
CAGGTTCAGGTGGTGCAGTCTGGAGCTGAGGTGAA
GAAGCCTGGGGCCTCAGTGAAGGTCTCCTGCAAGG
CTTCTGGTTACACCTTTTCCACCTTTGGTATCACC
TGGGTGCGACAGGCCCCTGGACAAGGGCTTGAATG
GATGGGATGGATCAGCGCTTACAATGGTGACACAA
ACTATGCACAGAATCTCCAGGGCAGAGTCATCATG
ACCACAGACACATCCACGAGCACAGCCTACATGGA
GCTGAGGAGCCTGAGATCTGACGACACGGCCGTTT
ATTACTGTGCGAGGAGCAGTGGCCACTACTACTAC
TACGGTATGGACGTCTGGGGCCAAGGGACCACGGT
CACCGTCTCCTCA
VH domain amino acid sequence:
SEQ ID NO: 366
QVQVVQSGAEVKKPGASVKVSCKASGYTFSTFGIT
WVRQAPGQGLEWMGWISAYNGDTNYAQNLQGRVIM
TTDTSTSTAYMELRSLRSDDTAVYYCARSSGHYYY
YGMDVWGQGTTVTVSS
VH CDR1 amino acid sequence:
SEQ ID NO: 363
GYTFSTFG
VH CDR2 amino acid sequence:
SEQ ID NO: 364
ISAYNGDT
VH CDR3 amino acid sequence:
SEQ ID NO: 365
ARSSGHYYYYGMDV
VL domain nucleotide sequence:
SEQ ID NO: 374
GATATTGTGATGACTCAGTCTCCACTCTCCCTGCC
CGTCACCCCTGGAGAGCCGGCCTCCATCTCCTGCA
GGTCTAGTCAGAGCCTCCTGCATAGTAATGAATAC
AACTATTTGGATTGGTACCTGCAGAAGCCAGGGCA
GTCTCCACAGCTCCTGATCTTTTTGGGTTCTAATC
GGGCCTCCGGGGTCCCTGACAGGTTCAGTGGCAGT
GGATCAGGCACAGATTTTACACTGAAAATCACCAG
AGTGGAGGCTGAGGATGTTGGAATTTATTACTGCA
TGCAATCTCTACAAACTCCGCTCACTTTCGGCGGA
GGGACCAAGGTGGAGATCAAA
VL domain amino acid sequence:
SEQ ID NO: 373
DIVMTQSPLSLPVTPGEPASISCRSSQSLLHSNEY
NYLDWYLQKPGQSPQLLIFLGSNRASGVPDRFSGS
GSGTDFTLKITRVEAEDVGIYYCMQSLQTPLTFGG
GTKVEIK
VL CDR1 amino acid sequence:
SEQ ID NO: 370
QSLLHSNEYNY
VL CDR2 amino acid sequence:
SEQ ID NO: 371
LGS
VL CDR3 amino acid sequence:
SEQ ID NO: 372
MQSLQTPLT
1.21.1.2. Antibody STIM002
VH domain nucleotide sequence:
SEQ ID NO: 381
CAGGTTCAACTGGTGCAGTCTGGAGGTGAGGTGAA
GAAGCCTGGGGCCTCAGTGAAGGTCTCCTGCAAGG
CTTCTGGTTACACCTTTACCAGCTATGGTTTCAGC
TGGGTGCGACAGGCCCCTGGACAAGGACTAGAGTG
GATGGGATGGATCAGCGCTTACAATGGTAACACAA
ACTATGCACAGAAGCTCCAGGGCAGAGTCACCATG
ACCACAGACACATCCACGAGCACAGCCTACATGGA
GCTGAGGAGCTTGAGATCTGACGACACGGCCGTGT
ATTACTGTGCGAGATCTACGTATTTCTATGGTTCG
GGGACCCTCTACGGTATGGACGTCTGGGGCCAAGG
GACCACGGTCACCGTCTCCTCA
VH domain amino acid sequence:
SEQ ID NO: 380
QVQLVQSGGEVKKPGASVKVSCKASGYTFTSYGFS
WVRQAPGQGLEWMGWISAYNGNTNYAQKLQGRVTM
TTDTSTSTAYMELRSLRSDDTAVYYCARSTYFYGS
GTLYGMDVWGQGTTVTVSS
VH CDR1 amino acid sequence:
SEQ ID NO: 377
GYTFTSYG
VH CDR2 amino acid sequence:
SEQ ID NO: 378
ISAYNGNT
VH CDR3 amino acid sequence:
SEQ ID NO: 379
ARSTYFYGSGTLYGMDV
VL domain nucleotide sequence: 388
GATATTGTGATGACTCAGTCTCCACTCTCCCTGCC
CGTCACCCCTGGAGAGCCGGCCTCCATCTCCTGCA
GGTCTAGTCAGAGCCTCCTGCATAGTGATGGATAC
AACTGTTTGGATTGGTACCTGCAGAAGCCAGGGCA
GTCTCCACAGCTCCTGATCTATTTGGGTTCTACTC
GGGCCTCCGGGTTCCCTGACAGGTTCAGTGGCAGT
GGATCAGGCACAGATTTTACACTGAAAATCAGCAG
AGTGGAGGCTGAGGATGTTGGGGTTTATTACTGCA
TGCAAGCTCTACAAACTCCGTGCAGTTTTGGCCAG
GGGACCAAGCTGGAGATCAAA
Corrected STIM002 VL domain nucleotide
sequence:
SEQ ID NO: 519
GATATTGTGATGACTCAGTCTCCACTCTCCCTGCC
CGTCACCCCTGGAGAGCCGGCCTCCATCTCCTGCA
GGTCTAGTCAGAGCCTCCTGCATAGTGATGGATAC
AACTATTTGGATTGGTACCTGCAGAAGCCAGGGCA
GTCTCCACAGCTCCTGATCTATTTGGGTTCTACTC
GGGCCTCCGGGTTCCCTGACAGGTTCAGTGGCAGT
GGATCAGGCACAGATTTTACACTGAAAATCAGCAG
AGTGGAGGCTGAGGATGTTGGGGTTTATTACTGCA
TGCAAGCTCTACAAACTCCGCTCAGTTTTGGCCAG
GGGACCAAGCTGGAGATCAAA
VL domain amino acid sequence:
SEQ ID NO: 387
DIVMTQSPLSLPVTPGEPASISCRSSQSLLHSDGY
NYLDWYLQKPGQSPQLLIYLGSTRASGFPDRFSGS
GSGTDFTLKISRVEAEDVGVYYCMQALQTPLSFGQ
GTKLEIK
VL CDR1 amino acid sequence:
SEQ ID NO: 384
QSLLHSDGYNY
VL CDR2 amino acid sequence:
SEQ ID NO: 385
LGS
VL CDR3 amino acid sequence:
SEQ ID NO: 386
MQALQTPLS
1.21.1.3. Antibody STIM002-B
VH domain nucleotide sequence:
SEQ ID NO: 395
CAGGTTCAACTGGTGCAGTCTGGAGGTGAGGTGAA
GAAGCCTGGGGCCTCAGTGAAGGTCTCCTGCAAGG
CTTCTGGTTACACCTTTACCAGCTATGGTTTCAGC
TGGGTGCGACAGGCCCCTGGACAAGGACTAGAGTG
GATGGGATGGATCAGCGCTTACAATGGTAACACAA
ACTATGCACAGAAGCTCCAGGGCAGAGTCACCATG
ACCACAGACACATCCACGAGCACAGCCTACATGGA
GCTGAGGAGCTTGAGATCTGACGACACGGCCGTGT
ATTACTGTGCGAGATCTACGTATTTCTATGGTTCG
GGGACCCTCTACGGTATGGACGTCTGGGGCCAAGG
GACCACGGTCACCGTCTCCTCA
VH domain amino acid sequence:
SEQ ID NO: 394
QVQLVQSGGEVKKPGASVKVSCKASGYTFTSYGFS
WVRQAPGQGLEWMGWISAYNGNTNYAQKLQGRVTM
TTDTSTSTAYMELRSLRSDDTAVYYCARSTYFYGS
GTLYGMDVWGQGTTVTVSS
VH CDR1 amino acid sequence:
SEQ ID NO: 391
GYTFTSYG
VH CDR2 amino acid sequence:
SEQ ID NO: 392
ISAYNGNT
VH CDR3 amino acid sequence:
SEQ ID NO: 393
ARSTYFYGSGTLYGMDV
VL domain nucleotide sequence:
SEQ ID NO: 402
GATATTGTGATGACTCAGTCTCCACTCTCCCTGCC
CGTCACCCCTGGAGAGCCGGCCTCCATCTCCTGCA
GGTCTAGTCAGAGCCTCCTGCATAGTGATGGATAC
AACTGTTTGGATTGGTACCTGCAGAAGCCAGGGCA
GTCTCCACAGCTCCTGATCTATTTGGGTTCTACTC
GGGCCTCCGGGTTCCCTGACAGGTTCAGTGGCAGT
GGATCAGGCACAGATTTTACACTGAAAATCAGCAG
AGTGGAGGCTGAGGATGTTGGGGTTTATTACTGCA
TGCAAGCTCTACAAACTCCGTGCAGTTTTGGCCAG
GGGACCAAGCTGGAGATCAAA
VL domain amino acid sequence:
SEQ ID NO: 401
DIVMTQSPLSLPVTPGEPASISCRSSQSLLHSDGY
NCLDWYLQKPGQSPQLLIYLGSTRASGFPDRFSGS
GSGTDFTLKISRVEAEDVGVYYCMQALQTPCSFGQ
GTKLEIK
VL CDR1 amino acid sequence:
SEQ ID NO: 398
QSLLHSDGYNC
VL CDR2 amino acid sequence:
SEQ ID NO: 399
LGS
VL CDR3 amino acid sequence:
SEQ ID NO: 400
MQALQTPCS
1.21.1.4. Antibody STIM003
VH domain nucleotide sequence:
SEQ ID NO: 409
GAGGTGCAGCTGGTGGAGTCTGGGGGAGGTGTGGT
ACGGCCTGGGGGGTCCCTGAGACTCTCCTGTGTAG
CCTCTGGAGTCACCTTTGATGATTATGGCATGAGC
TGGGTCCGCCAAGCTCCAGGGAAGGGGCTGGARTG
GGTCTCTGGTATTAATTGGAATGGTGGCGACACAG
ATTATTCAGACTCTGTGAAGGGCCGATTCACCATC
TCCAGAGACAACGCCAAGAACTCCCTGTATCTACA
AATGAATAGTCTGAGAGCCGAGGACACGGCCTTGT
ATTACTGTGCGAGGGATTTCTATGGTTCGGGGAGT
TATTATCACGTTCCTTTTGACTACTGGGGCCAGGG
AATCCTGGTCACCGTCTCCTCA
Corrected STIM003 VH domain nucleotide
sequence:
SEQ ID NO: 521
GAGGTGCAGCTGGTGGAGTCTGGGGGAGGTGTGGT
ACGGCCTGGGGGGTCCCTGAGACTCTCCTGTGTAG
CCTCTGGAGTCACCTTTGATGATTATGGCATGAGC
TGGGTCCGCCAAGCTCCAGGGAAGGGGCTGGAGTG
GGTCTCTGGTATTAATTGGAATGGTGGCGACACAG
ATTATTCAGACTCTGTGAAGGGCCGATTCACCATC
TCCAGAGACAACGCCAAGAACTCCCTGTATCTACA
AATGAATAGTCTGAGAGCCGAGGACACGGCCTTGT
ATTACTGTGCGAGGGATTTCTATGGTTCGGGGAGT
TATTATCACGTTCCTTTTGACTACTGGGGCCAGGG
AATCCTGGTCACCGTCTCCTCA
VH domain amino acid sequence:
SEQ ID NO: 408
EVQLVESGGGVVRPGGSLRLSCVASGVTFDDYGMS
WVRQAPGKGLEWVSGINWNGGDTDYSDSVKGRFTI
SRDNAKNSLYLQMNSLRAEDTALYYCARDFYGSGS
YYHVPFDYWGQGILVTVSS
VH CDR1 amino acid sequence:
SEQ ID NO: 405
GVTFDDYG
VH CDR2 amino acid sequence:
SEQ ID NO: 406
INWNGGDT
VH CDR3 amino acid sequence:
SEQ ID NO: 407
ARDFYGSGSYYHVPFDY
VL domain nucleotide sequence:
SEQ ID NO: 416
GAAATTGTGTTGACGCAGTCTCCAGGGACCCTGTC
TTTGTCTCCAGGGGAAAGAGCCACCCTCTCCTGCA
GGGCCAGTCAGAGTGTTAGCAGAAGCTACTTAGCC
TGGTACCAGCAGAAACGTGGCCAGGCTCCCAGGCT
CCTCATCTATGGTGCATCCAGCAGGGCCACTGGCA
TCCCAGACAGGTTCAGTGGCGATGGGTCTGGGACA
GACTTCACTCTCTCCATCAGCAGACTGGAGCCTGA
AGATTTTGCAGTGTATTACTGTCACCAGTATGATA
TGTCACCATTCACTTTCGGCCCTGGGACCAAAGTG
GATATCAAA
VL domain amino acid sequence:
SEQ ID NO: 415
EIVLTQSPGTLSLSPGERATLSCRASQSVSRSYLA
WYQQKRGQAPRLLIYGASSRATGIPDRFSGDGSGT
DFTLSISRLEPEDFAVYYCHQYDMSPFTFGPGTKV
DIK
VL CDR1 amino acid sequence:
SEQ ID NO: 412
QSVSRSY
VL CDR2 amino acid sequence:
SEQ ID NO: 413
GAS
VL CDR3 amino acid sequence:
SEQ ID NO: 414
HQYDMSPFT
1.21.1.5. Antibody STIM004
VH domain nucleotide sequence:
SEQ ID NO: 423
GAGGTGCAGCTGGTGGAGTCTGGGGGAGGTGTGGT
ACGGCCTGGGGGGTCCCTGAGACTCTCCTGTGCAG
CCTCTGGACTCACCTTTGATGATTATGGCATGAGC
TGGGTCCGCCAAGTTCCAGGGAAGGGGCTGGAGTG
GGTCTCTGGTATTAATTGGAATGGTGATAACACAG
ATTATGCAGACTCTGTGAAGGGCCGATTCACCATC
TCCAGAGACAACGCCAAGAACTCCCTGTATCTGCA
AATGAACAGTCTGAGAGCCGAGGACACGGCCTTGT
ATTACTGTGCGAGGGATTACTATGGTTCGGGGAGT
TATTATAACGTTCCTTTTGACTACTGGGGCCAGGG
AACCCTGGTCACCGTCTCCTCA
VH domain amino acid sequence:
SEQ ID NO: 422
EVQLVESGGGVVRPGGSLRLSCAASGLTFDDYGMS
WVRQVPGKGLEWVSGINWNGDNTDYADSVKGRFTI
SRDNAKNSLYLQMNSLRAEDTALYYCARDYYGSGS
YYNVPFDYWGQGTLVTVSS
VH CDR1 amino acid sequence:
SEQ ID NO: 419
GLTFDDYG
VH CDR2 amino acid sequence:
SEQ ID NO: 420
INWNGDNT
VH CDR3 amino acid sequence:
SEQ ID NO: 421
ARDYYGSGSYYNVPFDY
VL domain nucleotide sequence:
SEQ ID NO: 431
GAAATTGTGTTGACGCAGTCTCCAGGCACCCTGTC
TTTGTCTCCAGGGGAAAGAGCCACCCTCTCCTGCA
GGGCCAGTCAGAGTGTTAGCAGCAGCTACTTAGCC
TGGTACCAGCAGAAACCTGGCCAGGCTCCCAGGCT
CCTCATATATGGTGCATCCAGCAGGGCCACTGGCA
TCCCAGACAGGTTCAGTGGCAGTGGGTCTGGGACA
GACTTCACTCTCACCATCAGAAGACTGGAGCCTGA
AGATTTTGCAGTGTATTACTGTCAGCAGTATGGTA
GTTCACCATTCACTTCGGCCCTGGGACCAAAGTGG
ATATCAAA
VL domain amino acid sequence as encoded by
the above VL domain nucleotide sequence.
Corrected VL domain nucleotide sequence:
SEQ ID NO: 430
GAAATTGTGTTGACGCAGTCTCCAGGCACCCTGTC
TTTGTCTCCAGGGGAAAGAGCCACCCTCTCCTGCA
GGGCCAGTCAGAGTGTTAGCAGCAGCTACTTAGCC
TGGTACCAGCAGAAACCTGGCCAGGCTCCCAGGCT
CCTCATATATGGTGCATCCAGCAGGGCCACTGGCA
TCCCAGACAGGTTCAGTGGCAGTGGGTCTGGGACA
GACTTCACTCTCACCATCAGAAGACTGGAGCCTGA
AGATTTTGCAGTGTATTACTGTCAGCAGTATGGTA
GTTCACCATTCTTCGGCCCTGGGACCAAAGTGGAT
ATCAAA
Corrected VL domain amino acid sequence:
SEQ ID NO: 432
EIVLTQSPGTLSLSPGERATLSCRASQSVSSSYLA
WYQQKPGQAPRLLIYGASSRATGIPDRFSGSGSGT
DFTLTIRRLEPEDFAVYYCQQYGSSPFFGPGTKVD
IK
VL CDR1 amino acid sequence:
SEQ ID NO: 426
QSVSSSY
VL CDR2 amino acid sequence:
SEQ ID NO: 427
GAS
VL CDR3 amino acid sequence:
SEQ ID NO: 428
QQYGSSPF
1.21.1.6. Antibody STIM005
VH domain nucleotide sequence:
SEQ ID NO: 439
CAGGTTCAGTTGGTGCAGTCTGGAGCTGAGGTGAA
GAAGCCTGGGGCCTCAGTGAAGGTCTCCTGCAAGG
CTTCTGGTTACACCTTTAATAGTTATGGTATCATC
TGGGTGCGACAGGCCCCTGGACAAGGGCTTGAGTG
GATGGGATGGATCAGCGTTCACAATGGTAACACAA
ACTGTGCACAGAAGCTCCAGGGTAGAGTCACCATG
ACCACAGACACATCCACGAGCACAGCCTACATGGA
GCTGAGGAGCCTGAGAACTGACGACACGGCCGTGT
ATTACTGTGCGAGAGCGGGTTACGATATTTTGACT
GATTTTTCCGATGCTTTTGATATCTGGGGCCACGG
GACAATGGTCACCGTCTCTTCA
VH domain amino acid sequence:
SEQ ID NO: 438
QVQLVQSGAEVKKPGASVKVSCKASGYTFNSYGII
WVRQAPGQGLEWMGWISVHNGNTNCAQKLQGRVTM
TTDTSTSTAYMELRSLRTDDTAVYYCARAGYDILT
DFSDAFDIWGHGTMVTVSS
VH CDR1 amino acid sequence:
SEQ ID NO: 435
GYTFNSYG
VH CDR2 amino acid sequence:
SEQ ID NO: 436
ISVHNGNT
VH CDR3 amino acid sequence:
SEQ ID NO: 437
ARAGYDILTDFSDAFDI
VL domain nucleotide sequence:
SEQ ID NO: 446
GACATCCAGATGACCCAGTCTCCATCCTCCCTGTC
TGCATCTGTAGGAGACAGAGTCACCATCACTTGCC
GGGCAAGTCAGAACATTAATAACTTTTTAAATTGG
TATCAGCAGAAAGAAGGGAAAGGCCCTAAGCTCCT
GATCTATGCAGCATCCAGTTTGCAAAGAGGGATAC
CATCAACGTTCAGTGGCAGTGGATCTGGGACAGAC
TTCACTCTCACCATCAGCAGTCTGCAACCTGAAGA
TTTTGCAACTTACATCTGTCAACAGAGCTACGGTA
TCCCGTGGGTCGGCCAAGGGACCAAGGTGGAAATC
AAA
VL domain amino acid sequence:
SEQ ID NO: 445
DIQMTQSPSSLSASVGDRVTITCRASQNINNFLNW
YQQKEGKGPKLLIYAASSLQRGIPSTFSGSGSGTD
FTLTISSLQPEDFATYICQQSYGIPWVGQGTKVEI
K
VL CDR1 amino acid sequence:
SEQ ID NO: 442
QNINNF
VL CDR2 amino acid sequence:
SEQ ID NO: 443
AAS
VL CDR3 amino acid sequence:
SEQ ID NO: 444
QQSYGIPW
1.21.1.7. Antibody STIM006
VH domain nucleotide sequence:
SEQ ID NO: 453
CAGGTGCAGCTGGTGGAGTCTGGGGGAGGCTTGGT
CAAGCCTGGAGGGTCCCTGAGACTCTCCTGTGCAG
CCTCTGGATTCACCTTCAGTGACTACTTCATGAGC
TGGATCCGCCAGGCGCCAGGGAAGGGGCTGGAGTG
GATTTCATACATTAGTTCTAGTGGTAGTACCATAT
ACTACGCAGACTCTGTGAGGGGCCGATTCACCATC
TCCAGGGACAACGCCAAGTACTCACTGTATCTGCA
AATGAACAGCCTGAGATCCGAGGACACGGCCGTGT
ATTACTGTGCGAGAGATCACTACGATGGTTCGGGG
ATTTATCCCCTCTACTACTATTACGGTTTGGACGT
CTGGGGCCAGGGGACCACGGTCACCGTCTCCTCA
VH domain amino acid sequence:
SEQ ID NO: 454
QVQLVESGGGLVKPGGSLRLSCAASGFTFSDYFMS
WIRQAPGKGLEWISYISSSGSTIYYADSVRGRFTI
SRDNAKYSLYLQMNSLRSEDTAVYYCARDHYDGSG
IYPLYYYYGLDVWGQGTTVTVSS
VH CDR1 amino acid sequence:
SEQ ID NO: 449
GFTFSDYF
VH CDR2 amino acid sequence:
SEQ ID NO: 450
ISSSGSTI
VH CDR3 amino acid sequence:
SEQ ID NO: 451
ARDHYDGSGIYPLYYYYGLDV
VL domain nucleotide sequence:
SEQ ID NO: 460
ATTGTGATGACTCAGTCTCCACTCTCCCTACCCGT
CACCCCTGGAGAGCCGGCCTCCATCTCCTGCAGGT
CTAGTCAGAGCCTCCTGCATAGTAATGGATACAAC
TATTTGGATTATTACCTGCAGAAGCCAGGGCAGTC
TCCACAGCTCCTGATCTATTTGGGTTCTTATCGGG
CCTCCGGGGTCCCTGACAGGTTCAGTGGCAGTGGA
TCAGGCACAGATTTTACACTGAAAATCAGCAGAGT
GGAGGCTGAGGATGTTGGGGTTTATTACTGCATGC
AAGCTCTACAAACTCCTCGCAGTTTTGGCCAGGGG
ACCACGCTGGAGATCAAA
VL domain amino acid sequence:
SEQ ID NO: 459
IVMTQSPLSLPVTPGEPASISCRSSQSLLHSNGYN
YLDYYLQKPGQSPQLLIYLGSYRASGVPDRFSGSG
SGTDFTLKISRVEAEDVGVYYCMQALQTPRSFGQG
TTLEIK
VL CDR1 amino acid sequence:
SEQ ID NO: 456
QSLLHSNGYNY
VL CDR2 amino acid sequence:
SEQ ID NO: 457
LGS
VL CDR3 amino acid sequence:
SEQ ID NO: 458
MQALQTPRS
1.21.1.8. Antibody STIM007
VH domain nucleotide sequence:
SEQ ID NO: 467
CAGATCACCTTGAAGGAGTCTGGTCCTACGCTGGT
GAAACCCACACAGACCCTCACGCTGACCTGCACCT
TCTCTGGGTTCTCACTCAGCACTACTGGAGTGGGT
GTGGGCTGGATCCGTCAGCCCCCAGGAAAGGCCCT
GGAGTGGCTTGCAGTCATTTATTGGGATGATGATA
AGCGCTACAGCCCATCTCTGAAGAGCAGACTCACC
ATCACCAAGGACACCTCCAAAAACCAGGTGGTCCT
TACAATGACCAACATGGACCCTGTGGACACAGCCA
CATATTTCTGTACACACGGATATGGTTCGGCGAGT
TATTACCACTACGGTATGGACGTCTGGGGCCAAGG
GACCACGGTCACCGTCTCCTCA
VH domain amino acid sequence:
SEQ ID NO: 466
QITLKESGPTLVKPTQTLTLTCTFSGFSLSTTGVG
VGWIRQPPGKALEWLAVIYWDDDKRYSPSLKSRLT
ITKDTSKNQVVLTMTNMDPVDTATYFCTHGYGSAS
YYHYGMDVWGQGTTVTVSS
VH CDR1 amino acid sequence:
SEQ ID NO: 463
GFSLSTTGVG
VH CDR2 amino acid sequence:
SEQ ID NO: 464
IYWDDDK
VH CDR3 amino acid sequence:
SEQ ID NO: 465
THGYGSASYYHYGMDV
VL domain nucleotide sequence:
SEQ ID NO: 474
GAAATTGTATTGACACAGTCTCCAGCCACCCTGTC
TTTGTCTCCAGGGGAAAGAGCCACCCTCTCCTGCA
GGGCCAGTCAGAGTGTTACCAACTACTTAGCCTGG
CACCAACAGAAACCTGGCCAGGCTCCCAGGCTCCT
CATCTATGATGCATCCAACAGGGCCACTGGCATCC
CAGCCAGGTTCAGTGGCAGTGGGTCTGGGACAGAC
TTCACTCTCACCATCAGCAGCCTAGAGCCTGAAGA
TTTTGCAGTTTATTACTGTCAGCACCGTAGCAACT
GGCCTCTCACTTTCGGCGGAGGGACCAAGGTGGAG
ATCAAAC
VL domain amino acid sequence:
SEQ ID NO: 473
EIVLTQSPATLSLSPGERATLSCRASQSVTNYLAW
HQQKPGQAPRLLIYDASNRATGIPARFSGSGSGTD
FTLTISSLEPEDFAVYYCQHRSNWPLTFGGGTKVE
IK
VL CDR1 amino acid sequence:
SEQ ID NO: 470
QSVTNY
VL CDR2 amino acid sequence:
SEQ ID NO: 471
DAS
VL CDR3 amino acid sequence:
SEQ ID NO: 472
QHRSNWPLT
1.21.1.9. Antibody STIM008
VH domain nucleotide sequence:
SEQ ID NO: 481
CAGATCACCTTGAAGGAGTCTGGTCCTACGCTGGT
GAAACCCACACAGACCCTCACGCTGACCTGCACCT
TCTCTGGGTTCTCACTCAGCACTAGTGGAGTGGGT
GTGGGCTGGATCCGTCAGCCCCCAGGAAAGGCCCT
GGAGTGGCTTGCAGTCATTTATTGGGATGATGATA
AGCGCTACAGCCCATCTCTGAAGAGCAGGCTCACC
ATCACCAAGGACACCTCCAAAAACCAGGTGGTCCT
TACAATGACCAACATGGACCCTGTGGACACAGCCA
CATATTTCTGTACACACGGATATGGTTCGGCGAGT
TATTACCACTACGGTATGGACGTCTGGGGCCAAGG
GACCACGGTCACCGTCTCCTCA
VH domain amino acid sequence:
SEQ ID NO: 480
QITLKESGPTLVKPTQTLTLTCTFSGFSLSTSGVG
VGWIRQPPGKALEWLAVIYWDDDKRYSPSLKSRLT
ITKDTSKNQVVLTMTNMDPVDTATYFCTHGYGSAS
YYHYGMDVWGQGTTVTVSS
VH CDR1 amino acid sequence:
SEQ ID NO: 477
GFSLSTSGVG
VH CDR2 amino acid sequence:
SEQ ID NO: 478
IYWDDDK
VH CDR3 amino acid sequence:
SEQ ID NO: 479
THGYGSASYYHYGMDV
VL domain nucleotide sequence:
SEQ ID NO: 488
GAAATTGTGTTGACACAGTCTCCAGCCACCCTGTC
TTTGTCTCCAGGGGAAAGAGCCACCCTCTCCTGCA
GGGCCAGTCAGAGTGTTACCAACTACTTAGCCTGG
CACCAACAGAAACCTGGCCAGGCTCCCAGGCTCCT
CATCTATGATGCATCCAACAGGGCCACTGGCATCC
CAGCCAGGTTCAGTGGCAGTGGGTCTGGGACAGAC
TTCACTCTCACCATCAGCAGCCTAGAGCCTGAAGA
TTTTGCAGTTTATTACTGTCAGCAGCGTAGCAACT
GGCCTCTCACTTTCGGCGGAGGGACCAAGGTGGAG
ATCAAA
VL domain amino acid sequence:
SEQ ID NO: 489
EIVLTQSPATLSLSPGERATLSCRASQSVTNYLAW
HQQKPGQAPRLLIYDASNRATGIPARFSGSGSGTD
FTLTISSLEPEDFAVYYCQQRSNWPLTFGGGTKVE
IK
VL CDR1 amino acid sequence:
SEQ ID NO: 484
QSVTNY
VL CDR2 amino acid sequence:
SEQ ID NO: 485
DAS
VL CDR3 amino acid sequence:
SEQ ID NO: 486
QQRSNWPLT
1.21.1.10. Antibody STIM009
VH domain nucleotide sequence:
SEQ ID NO: 495
CAGGTGCAGCTGGTGGAGTCTGGGGGAGGCTTGGT
CAAGCCTGGAGGGTCCCTGAGACTCTCCTGTGCAG
CCTCTGGATTCACCTTCAGTGACTACTACATGAGC
TGGATCCGCCAGGCTCCAGGGAAGGGGCTGGAGTG
GGTTTCATACATTAGTAGTAGTGGTAGTACCATAT
ACTACGCAGACTCTGTGAAGGGCCGATTCACCATC
TCCAGGGACAACGCCAAGAACTCACTGTATCTGCA
AATTAACAGCCTGAGAGCCGAGGACACGGCCGTGT
ATTACTGTGCGAGAGATTTTTACGATATTTTGACT
GATAGTCCGTACTTCTACTACGGTGTGGACGTCTG
GGGCCAAGGGACCACGGTCACCGTCTCCTCA
VH domain amino acid sequence:
SEQ ID NO: 494
QVQLVESGGGLVKPGGSLRLSCAASGFTFSDYYMS
WIRQAPGKGLEWVSYISSSGSTIYYADSVKGRFTI
SRDNAKNSLYLQINSLRAEDTAVYYCARDFYDILT
DSPYFYYGVDVWGQGTTVTVSS
VH CDR1 amino acid sequence:
SEQ ID NO: 491
GFTFSDYY
VH CDR2 amino acid sequence:
SEQ ID NO: 492
ISSSGSTI
VH CDR3 amino acid sequence:
SEQ ID NO: 493
ARDFYDILTDSPYFYYGVDV
VL domain nucleotide sequence:
SEQ ID NO: 502
GATATTGTGATGACTCAGTCTCCACTCTCCCTGCC
CGTCACCCCTGGAGAGCCGGCCTCCATCTCCTGCA
GGTCTAGTCAGAGCCTCCTGCATAGTAATGGATAC
AACTATTTGGATTGGTACCTGCAGAAGCCAGGGCA
GTCTCCACAGCTCCTGATCTATTTGGGTTCTAATC
GGGCCTCCGGGGTCCCTGACAGGTTCAGTGGCAGT
GGATCAGGCACAGATTTTACACTGAAAATCAGCAG
AGTGGAGGCTGAGGATGTTGGGGTTTATTACTGCA
TGCAAGCTCTACAAACTCCTCGGACGTTCGGCCAA
GGGACCAAGGTGGAAATCAAA
VL domain amino acid sequence:
SEQ ID NO: 501
DIVMTQSPLSLPVTPGEPASISCRSSQSLLHSNGY
NYLDWYLQKPGQSPQLLIYLGSNRASGVPDRFSGS
GSGTDFTLKISRVEAEDVGVYYCMQALQTPRTFGQ
GTKVEIK
VL CDR1 amino acid sequence:
SEQ ID NO: 498
QSLLHSNGYNY
VL CDR2 amino acid sequence:
SEQ ID NO: 499
LGS
VL CDR3 amino acid sequence:
SEQ ID NO: 500
MQALQTPRT

TABLE S1
SEQ ID NOS: 1-342

| SEQ | | | | |
|---|---|---|---|---|
| ID | | | | |
| NO: | Name | Description | | Sequence |
| 1 | Human | NCBI number: | | MRIFAVFIFMTYWHLLNAFTVTVPKDLYVV |
| | PD-L1 | NP_054862.1 | | EYGSNMTIECKFPVEKQLDLAALIVYWEM |
| | | (ECD highlighted in | | EDKNIIQFVHGEEDLKVQHSSYRQRARLLK |
| | | BOLD, cytoplasmic | | DQLSLGNAALQITDVKLQDAGVYRCMISY |
| | | domain underlined) | | GGADYKRITVKVNAPYNKINQRILVVDPVT |
| | | | | SEHELTCQAEGYPKAEVIWTSSDHQVLSGK |
| | | | | TTTTNSKREEKLFNVTSTLRINTTTNEIFYC |
| | | | | TFRRLDPEENHTAELVIPELPLAHPPNERTH |
| | | | | LVILGAILLCLGVALTFIFRLRKGRMMDVKKC |
| | | | | GIQDTNSKKQSDTHLEET |
| 2 | Cyno | NCBI number: | | MGWSCIILFLVATATGVHSMFTVTVPKDLYV |
| | PD-L1 | XP_014973154.1 | | VEYGSNMTIECKFPVEKQLDLTSLIVYWE |
| | | (ECD highlighted in | | MEDKNIIQFVHGEEDLKVQHSNYRQRAQL |
| | | BOLD) | | LKDQLSLGNAALRITDVKLQDAGVYRCMI |
| | | | | SYGGADYKRITVKVNAPYNKINQRILVVDP |
| | | | | VTSEHELTCQAEGYPKAEVIWTSSDHQVLS |
| | | | | GKTTTTNSKREEKLLNVTSTLRINTTANEIF |
| | | | | YCIFRRLDPEENHTAELVIPELPLALPPNER |
| | | | | T |
| 3 | Human | Human PD-L1 ECD | | MRIFAVFIFMTYWHLLNAFTVTVPKDLYVVE |
| | PD-L1 | with C-terminal His | | YGSNMTIECKFPVEKQLDLAALIVYWEMEDK |
| | His | tag | | NIIQFVHGEEDLKVQHSSYRQRARLLKDQLSL |
| | | | | GNAALQITDVKLQDAGVYRCMISYGGADYK |
| | | | | RITVKVNAPYNKINQRILVVDPVTSEHELTCQ |
| | | | | AEGYPKAEVIWTSSDHQVLSGKTTTTNSKRE |
| | | | | EKLFNVTSTLRINTTTNEIFYCTFRRLDPEENH |
| | | | | TAELVIPELPLAHPPNERTHHHHHH |
| 4 | Human | Human PD-L1 ECD | | MRIFAVFIFMTYWHLLNAFTVTVPKDLYVVE |
| | PD-L1 | with C-term Fc | | YGSNMTIECKFPVEKQLDLAALIVYWEMEDK |
| | Fc | fusion (in bold) | | NIIQFVHGEEDLKVQHSSYRQRARLLKDQLSL |
| | | | | GNAALQITDVKLQDAGVYRCMISYGGADYK |
| | | | | RITVKVNAPYNKINQRILVVDPVTSEHELTCQ |
| | | | | AEGYPKAEVIWTSSDHQVLSGKTTTTNSKRE |
| | | | | EKLFNVTSTLRINTTTNEIFYCTFRRLDPEENH |
| | | | | TAELVIPELPLAHPPNERTIEGREPKSCDKTH |
| | | | | TCPPCPAPELLGGPSVFLFPPKPKDTLMISR |
| | | | | TPEVTCVVVDVSHEDPEVKFNWYVDGVEV |
| | | | | HNAKTKPREEQYNSTYRVVSVLTVLHQDW |
| | | | | LNGKEYKCKVSNKALPAPIEKTISKAKGQP |
| | | | | REPQVYTLPPSRDELTKNQVSLTCLVKGFY |
| | | | | PSDIAVEWESNGQPENNYKTTPPVLDSDGS |
| | | | | FFLYSKLTVDKSRWQQGNVFSCSVMHEAL |
| | | | | HNHYTQKSLSLSPGK |
| 5 | Cyno | Cynomolgus PD-L1 | | MGWSCIILFLVATATGVHSMFTVTVPKDLYV |
| | PD-L1 | ECD with N-term | | VEYGSNMTIECKFPVEKQLDLTSLIVYWEME |
| | FLAG | FLAG tag | | DKNIIQFVHGEEDLKVQHSNYRQRAQLLKDQ |
| | | | | LSLGNAALRITDVKLQDAGVYRCMISYGGAD |
| | | | | YKRITVKVNAPYNKINQRILVVDPVTSEHELT |
| | | | | CQAEGYPKAEVIWTSSDHQVLSGKTTTTNSK |
| | | | | REEKLLNVTSTLRINTTANEIFYCIFRRLDPEE |
| | | | | NHTAELVIPELPLALPPNERTDYKDDDDK |
| 6 | Human | Human PD-1 full | | MGWSCIILFLVATATGVHSLDSPDRPWNPPTF |
| | PD-1 Fc | length sequence | | SPALLVVTEGDNATFTCSFSNTSESFVLNWYR |
| | | derived from cDNA | | MSPSNQTDKLAAFPEDRSQPGQDCRFRVTQL |
| | | as human Fc fusion | | PNGRDFHMSVVRARRNDSGTYLCGAISLAPK |
| | | | | AQIKESLRAELRVTERRAEVPTAHPSPSPRPA |
| | | | | GQKLENLYFQGIEGRMDEPKSCDKTHTCP |
| | | | | PCPAPELLGGPSVFLFPPKPKDTLMISRTPE |
| | | | | VTCVVVDVSHEDPEVKFNWYVDGVEVHNA |
| | | | | KTKPREEQYNSTYRVVSVLTVLHQDWLNG |
| | | | | KEYKCKVSNKALPAPIEKTISKAKGQPREP |
| | | | | QVYTLPPSRDELTKNQVSLTCLVKGFYPSD |
| | | | | IAVEWESNGQPENNYKTTPPVLDSDGSFFL |
| | | | | YSKLTVDKSRWQQGNVFSCSVMHEALHN |
| | | | | HYTQKSLSLSP |
| 7 | 84G09- | Amino acid | | GFTFDDYA |
| | CDRH1 | sequence of CDRH1 | | |
| | (IMGT) | of 84G09 using | | |
| | | IMGT | | |
| 8 | 84G09- | Amino acid | | ISWKSNII |
| | CDRH2 | sequence of CDRH2 | | |
| | (IMGT) | of 84G09 using | | |
| | | IMGT | | |
| 9 | 84G09- | Amino acid | | ARDITGSGSYGWFDP |
| | CDRH3 | sequence of CDRH3 | | |
| | (IMGT) | of 84G09 using | | |
| | | IMGT | | |
| 10 | 84G09- | Amino acid | | DYAMH |
| | CDRH1 | sequence of CDRH1 | | |
| | (Kabat) | of 84G09 using | | |
| | | Kabat | | |
| 11 | 84G09- | Amino acid | | GISWKSNIIGYADSVKG |
| | CDRH2 | sequence of CDRH2 | | |
| | (Kabat) | of 84G09 using | | |
| | | Kabat | | |
| 12 | 84G09- | Amino acid | | DITGSGSYGWFDP |
| | CDRH3 | sequence of CDRH3 | | |
| | (Kabat) | of 84G09 using | | |
| | | Kabat | | |
| 13 | 84G09- | Amino acid | | EVQLVESGGGLVQPGRSLRLSCAASGFTFDD |
| | Heavy | sequence of V_{H} of | | YAMHWVRQTPGKGLEWVSGISWKSNIIGYA |
| | chain | 84G09(mutations | | DSVKGRFTISRDNAKNSLYLQMNSLRAEDTA |
| | variable | from germline are | | LYYCARDITGSGSYGWFDPWGQGTLVTVSS |
| | region | shown in bold | | |
| | | letters) | | |
| 14 | 84G09- | Nucleic acid | | CAaGAAAAAGCTTGCCGCCACCATGGAGTT |
| | Heavy | sequence of V_{H} of | | TGGGCTGAGCTGGATTTTCCTTTTGGCTATT |
| | chain | 84G09 | | TTAAAAGGTGTCCAGTGTGAAGTACAATTG |
| | variable | | | GTGGAGTCCGGGGGAGGCTTGGTACAGCCT |
| | region | | | GGCAGGTCCCTGAGACTCTCCTGTGCAGCC |
| | | | | TCTGGATTCACCTTTGATGATTATGCCATGC |
| | | | | ACTGGGTCCGACAAACTCCAGGGAAGGGCC |
| | | | | TGGAGTGGGTCTCAGGTATAAGTTGGAAGA |
| | | | | GTAATATCATAGGCTATGCGGACTCTGTGA |
| | | | | AGGGCCGATTCACCATCTCCAGAGACAACG |
| | | | | CCAAGAACTCCCTGTATCTGCAAATGAACA |
| | | | | GTCTGAGAGCTGAGGACACGGCCTTGTATT |
| | | | | ATTGTGCAAGAGATATAACGGGTTCGGGGA |
| | | | | GTTATGGCTGGTTCGACCCCTGGGGCCAGG |
| | | | | GAACCCTGGTCACCGTCTCCTCAGCCAAAA |
| | | | | CGACACCCCCATCTGTCTATCCACTGGCCCC |
| | | | | TGAATCTGCTAAAACTCAGCCTCCG |
| 15 | 84G09- | Amino acid | | EVQLVESGGGLVQPGRSLRLSCAASGFTFDD |
| | full | sequence of 84G09 | | YAMHWVRQTPGKGLEWVSGISWKSNIIGYA |
| | heavy | heavy chain | | DSVKGRFTISRDNAKNSLYLQMNSLRAEDTA |
| | chain | (mutations from | | LYYCARDITGSGSYGWFDPWGQGTLVTVSSA |
| | sequence | germline are shown | | STKGPSVFPLAPCSRSTSESTAALGCLVKDYF |
| | | in bold letters) | | PEPVTVSWNSGALTSGVHTFPAVLQSSGLYSL |
| | | | | SSVVTVPSSSLGTKTYTCNVDHKPSNTKVDK |
| | | | | RVESKYGPPCPPCPAPEFEGGPSVFLFPPKPK |
| | | | | DTLMISRTPEVTCVVVDVSQEDPEVQFNWYVD |
| | | | | GVEVHNAKTKPREEQFNSTYRVVSVLTVLHQ |
| | | | | DWLNGKEYKCKVSNKGLPSSIEKTISKAKGQ |
| | | | | PREPQVYTLPPSQEEMTKNQVSLTCLVKGFYP |
| | | | | SDIAVEWESNGQPENNYKTTPPVLDSDGSFFL |
| | | | | YSRLTVDKSRWQEGNVFSCSVMHEALHNHY |
| | | | | TQKSLSLSLGK |
| 16 | 84G09- | Nucleic acid | | GAAGTGCAGCTGGTGGAATCTGGCGGCGGA |
| | full | sequence of 84G09 | | CTGGTGCAGCCTGGCAGATCCCTGAGACTG |
| | heavy | heavy chain | | TCTTGTGCCGCCTCCGGCTTCACCTTCGACG |
| | chain | | | ACTACGCTATGCACTGGGTGCGACAGACCC |
| | sequence | | | CTGGCAAGGGCCTGGAATGGGTGTCCGGCA |
| | | | | TCTCCTGGAAGTCCAACATCATCGGCTACG |
| | | | | CCGACTCCGTGAAGGGCCGGTTCACCATCT |
| | | | | CCCGGGACAACGCCAAGAACTCCCTGTACC |
| | | | | TGCAGATGAACAGCCTGCGGGCCGAGGAC |
| | | | | ACCGCCCTGTACTACTGCGCCAGAGACATC |
| | | | | ACCGGCTCCGGCTCCTACGGATGGTTCGAT |
| | | | | CCTTGGGGCCAGGGCACCCTCGTGACCGTG |
| | | | | TCCTCTGCCAGCACCAAGGGCCCCTCTGTG |
| | | | | TTCCCTCTGGCCCCTTCCAGCAAGTCCACCT |
| | | | | CTGGCGGAACAGCCGCTCTGGGCTGCCTCG |
| | | | | TGAAGGACTACTTCCCCGAGCCTGTGACCG |
| | | | | TGTCCTGGAACTCTGGCGCTCTGACCAGCG |
| | | | | GAGTGCACACCTTCCCTGCTGTGCTGCAGT |
| | | | | CCTCCGGCCTGTACTCCCTGTCCTCCGTCGT |
| | | | | GACCGTGCCTTCCAGCTCTCTGGGCACCCA |
| | | | | GACCTACATCTGCAACGTGAACCACAAGCC |
| | | | | CTCCAACACCAAGGTGGACAAGAAGGTGG |
| | | | | AACCCAAGTCCTGCGACAAGACCCACACCT |
| | | | | GTCCCCCTTGTCCTGCCCCTGAACTGCTGGG |
| | | | | CGGACCTTCCGTGTTCCTGTTCCCCCCAAAG |
| | | | | CCCAAGGACACCCTGATGATCTCCCGGACC |
| | | | | CCCGAAGTGACCTGCGTGGTGGTGGATGTG |
| | | | | TCCCACGAGGACCCTGAAGTGAAGTTCAAT |
| | | | | TGGTACGTGGACGGCGTGGAAGTGCACAAC |
| | | | | GCCAAGACCAAGCCTAGAGAGGAACAGTA |
| | | | | CAACTCCACCTACCGGGTGGTGTCCGTGCT |
| | | | | GACCGTGCTGCACCAGGATTGGCTGAACGG |
| | | | | CAAAGAGTACAAGTGCAAGGTGTCCAACA |
| | | | | AGGCCCTGCCTGCCCCCATCGAAAAGACCA |
| | | | | TCTCCAAGGCCAAGGGCCAGCCCCGGGAAC |
| | | | | CCCAGGTGTACACACTGCCCCCTAGCAGGG |
| | | | | ACGAGCTGACCAAGAACCAGGTGTCCCTGA |
| | | | | CCTGTCTCGTGAAAGGCTTCTACCCCTCCGA |
| | | | | TATCGCCGTGGAATGGGAGTCCAACGGCCA |
| | | | | GCCTGAGAACAACTACAAGACCACCCCCCC |
| | | | | TGTGCTGGACTCCGACGGCTCATTCTTCCTG |
| | | | | TACAGCAAGCTGACAGTGGACAAGTCCCGG |
| | | | | TGGCAGCAGGGCAACGTGTTCTCCTGCTCC |
| | | | | GTGATGCACGAGGCCCTGCACAACCACTAC |
| | | | | ACCCAGAAGTCCCTGTCCCTGAGCCCCGGC |
| | | | | AAG |
| 17 | 84G09- | Amino acid | | QSISSY |
| | CDRL1 | sequence of CDRL1 | | |
| | (IMGT) | of 84G09 using | | |
| | | IMGT | | |
| 18 | 84G09- | Amino acid | | VAS |
| | CDRL2 | sequence of CDRL2 | | |
| | (IMGT) | of 84G09 using | | |
| | | IMGT | | |
| 19 | 84G09- | Amino acid | | QQSYSNPIT |
| | CDRL3 | sequence of CDRL3 | | |
| | (IMGT) | of 84G09 using | | |
| | | IMGT | | |
| 20 | 84G09- | Amino acid | | RASQSISSYLN |
| | CDRL1 | sequence of CDRL1 | | |
| | (Kabat) | of 84G09 using | | |
| | | Kabat | | |
| 21 | 84G09- | Amino acid | | VASSLQS |
| | CDRL2 | sequence of CDRL2 | | |
| | (Kabat) | of 84G09 using | | |
| | | Kabat | | |
| 22 | 84G09- | Amino acid | | QQSYSNPIT |
| | CDRL3 | sequence of CDRL3 | | |
| | (Kabat) | of 84G09 using | | |
| | | Kabat | | |
| 23 | 84G09- | Amino acid | | DIQMTQSPSSLSASVGDRVTITCRASQSISSYL |
| | Light | sequence of V_{L} of | | NWYQQKPGKAPKPLIYVASSLQSGVPSSFSGS |
| | chain | 84G09 | | GSGTDFTLTISSLQPEDFATYYCQQSYSNPITF |
| | variable | | | GQGTRLEIK |
| | region | | | |
| 24 | 84G09- | Nucleic acid | | GACATCCAGATGACCCAGTCTCCATCCTCC |
| | Light | sequence of V_{L} of | | CTGTCTGCATCTGTAGGAGACAGAGTCACC |
| | chain | 84G09 | | ATCACTTGCCGGGCAAGTCAGAGCATTAGC |
| | variable | | | AGCTATTTAAATTGGTATCAGCAGAAACCA |
| | region | | | GGGAAAGCCCCTAAGCCCCTGATCTATGTT |
| | | | | GCATCCAGTTTGCAAAGTGGGGTCCCATCA |
| | | | | AGTTTCAGTGGCAGTGGATCTGGGACAGAT |
| | | | | TTCACTCTCACCATCAGCAGTCTGCAACCTG |
| | | | | AAGATTTTGCAACTTACTACTGTCAACAGA |
| | | | | GTTACAGTAATCCGATCACCTTCGGCCAAG |
| | | | | GGACACGACTGGAGATCAAA |
| 25 | 84G09- | Amino acid | | DIQMTQSPSSLSASVGDRVTITCRASQSISSYL |
| | full light | sequence of 84G09 | | NWYQQKPGKAPKPLIYVASSLQSGVPSSFSGS |
| | chain | light chain | | GSGTDFTLTISSLQPEDFATYYCQQSYSNPITF |
| | sequence | | | GQGTRLEIKRTVAAPSVFIFPPSDEQLKSGTAS |
| | | | | VVCLLNNFYPREAKVQWKVDNALQSGNSQE |
| | | | | SVTEQDSKDSTYSLSSTLTLSKADYEKHKVY |
| | | | | ACEVTHQGLSSPVTKSFNRGEC |
| 26 | 84G09- | Nucleic acid | | GACATCCAGATGACCCAGTCTCCATCCTCC |
| | full light | sequence of 84G09 | | CTGTCTGCATCTGTAGGAGACAGAGTCACC |
| | chain | light chain | | ATCACTTGCCGGGCAAGTCAGAGCATTAGC |
| | sequence | | | AGCTATTTAAATTGGTATCAGCAGAAACCA |
| | | | | GGGAAAGCCCCTAAGCCCCTGATCTATGTT |
| | | | | GCATCCAGTTTGCAAAGTGGGGTCCCATCA |
| | | | | AGTTTCAGTGGCAGTGGATCTGGGACAGAT |
| | | | | TTCACTCTCACCATCAGCAGTCTGCAACCTG |
| | | | | AAGATTTTGCAACTTACTACTGTCAACAGA |
| | | | | GTTACAGTAATCCGATCACCTTCGGCCAAG |
| | | | | GGACACGACTGGAGATCAAACGTACGGTG |
| | | | | GCCGCTCCCTCCGTGTTCATCTTCCCACCTT |
| | | | | CCGACGAGCAGCTGAAGTCCGGCACCGCTT |
| | | | | CTGTCGTGTGCCTGCTGAACAACTTCTACCC |
| | | | | CCGCGAGGCCAAGGTGCAGTGGAAGGTGG |
| | | | | ACAACGCCCTGCAGTCCGGCAACTCCCAGG |
| | | | | AATCCGTGACCGAGCAGGACTCCAAGGACA |
| | | | | GCACCTACTCCCTGTCCTCCACCCTGACCCT |
| | | | | GTCCAAGGCCGACTACGAGAAGCACAAGG |
| | | | | TGTACGCCTGCGAAGTGACCCACCAGGGCC |
| | | | | TGTCTAGCCCCGTGACCAAGTCTTTCAACC |
| | | | | GGGGCGAGTGT |
| 27 | 1D05- | Amino acid | | GFTFDDYA |
| | CDRH1 | sequence of CDRH1 | | |
| | (IMGT) | of 1D05 using | | |
| | | IMGT | | |
| 28 | 1D05- | Amino acid | | ISWIRTGI |
| | CDRH2 | sequence of CDRH2 | | |
| | (IMGT) | of 1D05 using | | |
| | | IMGT | | |
| 29 | 1D05- | Amino acid | | AKDMKGSGTYGGWFDT |
| | CDRH3 | sequence of CDRH3 | | |
| | (IMGT) | of 1D05 using | | |
| | | IMGT | | |
| 30 | 1D05- | Amino acid | | DYAMH |
| | CDRH1 | sequence of CDRH1 | | |
| | (Kabat) | of 1D05 using Kabat | | |
| 31 | 1D05- | Amino acid | | GISWIRTGIGYADSVKG |
| | CDRH2 | sequence of CDRH2 | | |
| | (Kabat) | of 1D05 using Kabat | | |
| 32 | 1D05- | Amino acid | | DMKGSGTYGGWFDT |
| | CDRH3 | sequence of CDRH3 | | |
| | (Kabat) | of 1D05 using Kabat | | |
| 33 | 1D05- | Amino acid | | EVQLVESGGGLVQPGRSLRLSCAASGFTFDD |
| | Heavy | sequence of V_{H} of | | YAMHWVRQVPGKGLEWVSGISWIRTGIGYA |
| | chain | 1D05 (mutations | | DSVKGRFTIFRDNAKNSLYLQMNSLRAEDTA |
| | variable | from germline are | | LYYCAKDMKGSGTYGGWFDTWGQGTLVTV |
| | region | shown in bold | | SS |
| | | letters) | | |
| 34 | 1D05- | Nucleic acid | | AAGCTTGCCGCCACCATGGAGTTTGGGCTG |
| | Heavy | sequence of V_{H} of | | AGCTGGATTTTCCTTTTGGCTATTTTAAAAG |
| | chain | 1D05 | | GTGTCCAGTGTGAAGTGCAGCTGGTGGAGT |
| | variable | | | CTGGGGGAGGCTTGGTGCAGCCTGGCAGGT |
| | region | | | CCCTGAGACTCTCCTGTGCAGCCTCTGGATT |
| | | | | CACCTTTGATGATTATGCCATGCACTGGGTC |
| | | | | CGGCAAGTTCCAGGGAAGGGCCTGGAATG |
| | | | | GGTCTCAGGCATTAGTTGGATTCGTACTGG |
| | | | | CATAGGCTATGCGGACTCTGTGAAGGGCCG |
| | | | | ATTCACCATTTTCAGAGACAACGCCAAGAA |
| | | | | TTCCCTGTATCTGCAAATGAACAGTCTGAG |
| | | | | AGCTGAGGACACGGCCTTGTATTACTGTGC |
| | | | | AAAAGATATGAAGGGTTCGGGGACTTATGG |
| | | | | GGGGTGGTTCGACACCTGGGGCCAGGGAAC |
| | | | | CCTGGTCACCGTCTCCTCAGCCAAAACAAC |
| | | | | AGCCCCATCGGTCTATCCACTGGCCCCTGC |
| 35 | 1D05- | Amino acid | | EVQLVESGGGLVQPGRSLRLSCAASGFTFDD |
| | full | sequence of 1D05 | | YAMHWVRQVPGKGLEWVSGISWIRTGIGYA |
| | heavy | heavy chain | | DSVKGRFTIFRDNAKNSLYLQMNSLRAEDTA |
| | chain | | | LYYCAKDMKGSGTYGGWFDTWGQGTLVTV |
| | sequence | | | SSASTKGPSVFPLAPCSRSTSESTAALGCLVK |
| | | | | DYFPEPVTVSWNSGALTSGVHTFPAVLQSSG |
| | | | | LYSLSSVVTVPSSSLGTKTYTCNVDHKPSNTK |
| | | | | VDKRVESKYGPPCPPCPAPEFEGGPSVFLFPP |
| | | | | KPKDTLMISRTPEVTCVVVDVSQEDPEVQFN |
| | | | | WYVDGVEVHNAKTKPREEQFNSTYRVVSVL |
| | | | | TVLHQDWLNGKEYKCKVSNKGLPSSIEKTIS |
| | | | | KAKGQPREPQVYTLPPSQEEMTKNQVSLTCL |
| | | | | VKGFYPSDIAVEWESNGQPENNYKTTPPVLD |
| | | | | SDGSFFLYSRLTVDKSRWQEGNVFSCSVMHE |
| | | | | ALHNHYTQKSLSLSLGK |
| 36 | 1D05- | Nucleic acid | | GAAGTGCAGCTGGTGGAATCTGGCGGCGGA |
| | full | sequence of 1D05 | | CTGGTGCAGCCTGGCAGATCCCTGAGACTG |
| | heavy | heavy chain | | TCTTGTGCCGCCTCCGGCTTCACCTTCGACG |
| | chain | | | ACTACGCTATGCACTGGGTGCGACAGGTGC |
| | sequence | | | CAGGCAAGGGCCTGGAATGGGTGTCCGGCA |
| | | | | TCTCTTGGATCCGGACCGGCATCGGCTACG |
| | | | | CCGACTCTGTGAAGGGCCGGTTCACCATCT |
| | | | | TCCGGGACAACGCCAAGAACTCCCTGTACC |
| | | | | TGCAGATGAACAGCCTGCGGGCCGAGGAC |
| | | | | ACCGCCCTGTACTACTGCGCCAAGGACATG |
| | | | | AAGGGCTCCGGCACCTACGGCGGATGGTTC |
| | | | | GATACTTGGGGCCAGGGCACCCTCGTGACC |
| | | | | GTGTCCTCTGCCAGCACCAAGGGCCCCTCT |
| | | | | GTGTTCCCTCTGGCCCCTTCCAGCAAGTCCA |
| | | | | CCTCTGGCGGAACAGCCGCTCTGGGCTGCC |
| | | | | TCGTGAAGGACTACTTCCCCGAGCCTGTGA |
| | | | | CCGTGTCCTGGAACTCTGGCGCTCTGACCA |
| | | | | GCGGAGTGCACACCTTCCCTGCTGTGCTGC |
| | | | | AGTCCTCCGGCCTGTACTCCCTGTCCTCCGT |
| | | | | CGTGACCGTGCCTTCCAGCTCTCTGGGCAC |
| | | | | CCAGACCTACATCTGCAACGTGAACCACAA |
| | | | | GCCCTCCAACACCAAGGTGGACAAGAAGGT |
| | | | | GGAACCCAAGTCCTGCGACAAGACCCACAC |
| | | | | CTGTCCCCCTTGTCCTGCCCCTGAACTGCTG |
| | | | | GGCGGACCTTCCGTGTTCCTGTTCCCCCCAA |
| | | | | AGCCCAAGGACACCCTGATGATCTCCCGGA |
| | | | | CCCCCGAAGTGACCTGCGTGGTGGTGGATG |
| | | | | TGTCCCACGAGGACCCTGAAGTGAAGTTCA |
| | | | | ATTGGTACGTGGACGGCGTGGAAGTGCACA |
| | | | | ACGCCAAGACCAAGCCTAGAGAGGAACAG |
| | | | | TACAACTCCACCTACCGGGTGGTGTCCGTG |
| | | | | CTGACCGTGCTGCACCAGGATTGGCTGAAC |
| | | | | GGCAAAGAGTACAAGTGCAAGGTGTCCAA |
| | | | | CAAGGCCCTGCCTGCCCCCATCGAAAAGAC |
| | | | | CATCTCCAAGGCCAAGGGCCAGCCCCGGGA |
| | | | | ACCCCAGGTGTACACACTGCCCCCTAGCAG |
| | | | | GGACGAGCTGACCAAGAACCAGGTGTCCCT |
| | | | | GACCTGTCTCGTGAAAGGCTTCTACCCCTCC |
| | | | | GATATCGCCGTGGAATGGGAGTCCAACGGC |
| | | | | CAGCCTGAGAACAACTACAAGACCACCCCC |
| | | | | CCTGTGCTGGACTCCGACGGCTCATTCTTCC |
| | | | | TGTACAGCAAGCTGACAGTGGACAAGTCCC |
| | | | | GGTGGCAGCAGGGCAACGTGTTCTCCTGCT |
| | | | | CCGTGATGCACGAGGCCCTGCACAACCACT |
| | | | | ACACCCAGAAGTCCCTGTCCCTGAGCCCCG |
| | | | | GCAAG |
| 37 | 1D05- | Amino acid | | QSISSY |
| | CDRL1 | sequence of CDRL1 | | |
| | (IMGT) | of 1D05 using | | |
| | | IMGT | | |
| 38 | 1D05- | Amino acid | | VAS |
| | CDRL2 | sequence of CDRL2 | | |
| | (IMGT) | of 1D05 using | | |
| | | IMGT | | |
| 39 | 1D05- | Amino acid | | QQSYSTPIT |
| | CDRL3 | sequence of CDRL3 | | |
| | (IMGT) | of 1D05 using | | |
| | | IMGT | | |
| 40 | 1D05- | Amino acid | | RASQSISSYLN |
| | CDRL1 | sequence of CDRL1 | | |
| | (Kabat) | of 1D05 using Kabat | | |
| 41 | 1D05- | Amino acid | | VASSLQS |
| | CDRL2 | sequence of CDRL2 | | |
| | (Kabat) | of 1D05 using Kabat | | |
| 42 | 1D05- | Amino acid | | QQSYSTPIT |
| | CDRL3 | sequence of CDRL3 | | |
| | (Kabat) | of 1D05 using Kabat | | |
| 43 | 1D05- | Amino acid | | DIQMTQSPSSLSASVGDRVTITCRASQSISSYL |
| | Light | sequence of V_{L} of | | NWYQQKPGKAPKLLIYVASSLQSGVPSRFSG |
| | chain | 1D05 (mutations | | SGSGTDFTLTISSLQPEDFATYYCQQSYSTPIT |
| | variable | from germline are | | FGQGTRLEIK |
| | region | shown in bold | | |
| | | letters) | | |
| 44 | 1D05- | Nucleic acid | | AAAGCTTGCCGCCACCATGAGGCTCCCTGC |
| | Light | sequence of V_{L} of | | TCAGCTTCTGGGGCTCCTGCTACTCTGGCTC |
| | chain | 1D05 | | CGAGGTGCCAGATGTGACATCCAGATGACC |
| | variable | | | CAGTCTCCATCCTCCCTGTCTGCATCTGTAG |
| | region | | | GAGACAGAGTCACCATCACTTGCCGGGCAA |
| | | | | GTCAGAGCATTAGCAGCTATTTAAATTGGT |
| | | | | ATCAGCAGAAACCAGGGAAAGCCCCTAAA |
| | | | | CTCCTGATCTATGTTGCATCCAGTTTGCAAA |
| | | | | GTGGGGTCCCATCAAGGTTCAGTGGCAGTG |
| | | | | GATCTGGGACAGATTTCACTCTCACTATCA |
| | | | | GCAGTCTGCAACCTGAAGATTTTGCAACTT |
| | | | | ACTACTGTCAACAGAGTTACAGTACCCCGA |
| | | | | TCACCTTCGGCCAAGGGACACGTCTGGAGA |
| | | | | TCAAACGTACGGATGCTGCACCAACT |
| 45 | 1D05- | Amino acid | | DIQMTQSPSSLSASVGDRVTITCRASQSISSYL |
| | full light | sequence of 1D05 | | NWYQQKPGKAPKLLIYVASSLQSGVPSRFSG |
| | chain | light chain | | SGSGTDFTLTISSLQPEDFATYYCQQSYSTPIT |
| | sequence | | | FGQGTRLEIKRTVAAPSVFIFPPSDEQLKSGTA |
| | | | | SVVCLLNNFYPREAKVQWKVDNALQSGNSQ |
| | | | | ESVTEQDSKDSTYSLSSTLTLSKADYEKHKVY |
| | | | | ACEVTHQGLSSPVTKSFNRGEC |
| 46 | 1D05- | Nucleic acid | | GACATCCAGATGACCCAGTCCCCCTCCAGC |
| | full light | sequence of 1D05 | | CTGTCTGCTTCCGTGGGCGACAGAGTGACC |
| | chain | light chain | | ATCACCTGTCGGGCCTCCCAGTCCATCTCCT |
| | sequence | | | CCTACCTGAACTGGTATCAGCAGAAGCCCG |
| | | | | GCAAGGCCCCCAAGCTGCTGATCTACGTGG |
| | | | | CCAGCTCTCTGCAGTCCGGCGTGCCCTCTA |
| | | | | GATTCTCCGGCTCTGGCTCTGGCACCGACTT |
| | | | | TACCCTGACCATCAGCTCCCTGCAGCCCGA |
| | | | | GGACTTCGCCACCTACTACTGCCAGCAGTC |
| | | | | CTACTCCACCCCTATCACCTTCGGCCAGGG |
| | | | | CACCCGGCTGGAAATCAAACGTACGGTGGC |
| | | | | CGCTCCCTCCGTGTTCATCTTCCCACCTTCC |
| | | | | GACGAGCAGCTGAAGTCCGGCACCGCTTCT |
| | | | | GTCGTGTGCCTGCTGAACAACTTCTACCCCC |
| | | | | GCGAGGCCAAGGTGCAGTGGAAGGTGGAC |
| | | | | AACGCCCTGCAGTCCGGCAACTCCCAGGAA |
| | | | | TCCGTGACCGAGCAGGACTCCAAGGACAGC |
| | | | | ACCTACTCCCTGTCCTCCACCCTGACCCTGT |
| | | | | CCAAGGCCGACTACGAGAAGCACAAGGTG |
| | | | | TACGCCTGCGAAGTGACCCACCAGGGCCTG |
| | | | | TCTAGCCCCGTGACCAAGTCTTTCAACCGG |
| | | | | GGCGAGTGT |
| 47 | Mutated | Amino acid | | EVQLVESGGGLVQPGRSLRLSCAASGFTFDD |
| | 1D05- | sequence of 1D05 | | YAMHWVRQAPGKGLEWVSGISWIRTGIGYA |
| | HC | heavy chain with V | | DSVKGRFTIFRDNAKNSLYLQMNSLRAEDTA |
| | mutant 1 | to A back-mutation | | LYYCAKDMKGSGTYGGWFDTWGQGTLVTV |
| | | in framework region | | SSASTKGPSVFPLAPCSRSTSESTAALGCLVK |
| | | to germline | | DYFPEPVTVSWNSGALTSGVHTFPAVLQSSG |
| | | highlighted with | | LYSLSSVVTVPSSSLGTKTYTCNVDHKPSNTK |
| | | IgG1 disabled | | VDKRVESKYGPPCPPCPAPELAGAPSVFLFPP |
| | | (LAGA) constant | | KPKDTLMISRTPEVTCVVVDVSQEDPEVQFN |
| | | region | | WYVDGVEVHNAKTKPREEQFNSTYRVVSVL |
| | | | | TVLHQDWLNGKEYKCKVSNKGLPSSIEKTIS |
| | | | | KAKGQPREPQVYTLPPSQEEMTKNQVSLTCL |
| | | | | VKGFYPSDIAVEWESNGQPENNYKTTPPVLD |
| | | | | SDGSFFLYSRLTVDKSRWQEGNVFSCSVMHE |
| | | | | ALHNHYTQKSLSLSLGK |
| 48 | Mutated | Amino acid | | EVQLVESGGGLVQPGRSLRLSCAASGFTFDD |
| | 1D05- | sequence of 1D05 | | YAMHWVRQVPGKGLEWVSGISWIRTGIGYA |
| | HC | heavy chain with F | | DSVKGRFTISRDNAKNSLYLQMNSLRAEDTA |
| | mutant 2 | to S back-mutation | | LYYCAKDMKGSGTYGGWFDTWGQGTLVTV |
| | | in framework region | | SSASTKGPSVFPLAPCSRSTSESTAALGCLVK |
| | | to germline | | DYFPEPVTVSWNSGALTSGVHTFPAVLQSSG |
| | | highlighted with | | LYSLSSVVTVPSSSLGTKTYTCNVDHKPSNTK |
| | | IgG1 disabled | | VDKRVESKYGPPCPPCPAPELAGAPSVFLFPP |
| | | (LAGA) constant | | KPKDTLMISRTPEVTCVVVDVSQEDPEVQFN |
| | | region | | WYVDGVEVHNAKTKPREEQFNSTYRVVSVL |
| | | | | TVLHQDWLNGKEYKCKVSNKGLPSSIEKTIS |
| | | | | KAKGQPREPQVYTLPPSQEEMTKNQVSLTCL |
| | | | | VKGFYPSDIAVEWESNGQPENNYKTTPPVLD |
| | | | | SDGSFFLYSRLTVDKSRWQEGNVFSCSVMHE |
| | | | | ALHNHYTQKSLSLSLGK |
| 49 | Mutated | Amino acid | | EVQLVESGGGLVQPGRSLRLSCAASGFTFDD |
| | 1D05- | sequence of 1D05 | | YAMHWVRQVPGKGLEWVSGISWIRTGIGYA |
| | HC | heavy chain with | | DSVKGRFTIFRDNAKNSLYLQMNSLRAEDTA |
| | mutant 3 | ELLG to-PVA | | LYYCAKDMKGSGTYGGWFDTWGQGTLVTV |
| | | back-mutation in | | SSASTKGPSVFPLAPCSRSTSESTAALGCLVK |
| | | constant region to | | DYFPEPVTVSWNSGALTSGVHTFPAVLQSSG |
| | | germline highlighted | | LYSLSSVVTVPSSSLGTKTYTCNVDHKPSNTK |
| | | | | VDKRVESKYGPPCPPCPAP- |
| | | | | PVAGPSVFLFPPKPKDTLMISRTPEVTCVVVD |
| | | | | VSQEDPEVQFNWYVDGVEVHNAKTKPREEQ |
| | | | | FNSTYRVVSVLTVLHQDWLNGKEYKCKVSN |
| | | | | KGLPSSIEKTISKAKGQPREPQVYTLPPSQEEM |
| | | | | TKNQVSLTCLVKGFYPSDIAVEWESNGQPEN |
| | | | | NYKTTPPVLDSDGSFFLYSRLTVDKSRWQEG |
| | | | | NVFSCSVMHEALHNHYTQKSLSLSLGK |
| 50 | Mutated | Amino acid | | DIQMTQSPSSLSASVGDRVTITCRASQSISSYL |
| | 1D05- | sequence of 1D05 | | NWYQQKPGKAPKLLIYAASSLQSGVPSRFSG |
| | LC | kappa light chain | | SGSGTDFTLTISSLQPEDFATYYCQQSYSTPIT |
| | mutant 1 | with V to A back- | | FGQGTRLEIKRTVAAPSVFIFPPSDEQLKSGTA |
| | | mutation in CDRL2 | | SVVCLLNNFYPREAKVQWKVDNALQSGNSQ |
| | | to germline | | ESVTEQDSKDSTYSLSSTLTLSKADYEKHKVY |
| | | highlighted | | ACEVTHQGLSSPVTKSFNRGEC |
| 51 | Mutated | Amino acid | | DIQMTQSPSSLSASVGDRVTITCRASQSISSYL |
| | 1D05- | sequence of 1D05 | | NWYQQKPGKAPKLFIYVASSLQSGVPSRFSG |
| | LC | kappa light chain | | SGSGTDFTLTISSLQPEDFATYYCQQSYSTPIT |
| | mutant 2 | with L to F back- | | FGQGTRLEIKRTVAAPSVFIFPPSDEQLKSGTA |
| | | mutation in | | SVVCLLNNFYPREAKVQWKVDNALQSGNSQ |
| | | framework to | | ESVTEQDSKDSTYSLSSTLTLSKADYEKHKVY |
| | | germline highlighted | | ACEVTHQGLSSPVTKSFNRGEC |
| 52 | 411B08 | Amino acid | | GFTFSSYW |
| | CDRH1 | sequence of CDRH1 | | |
| | (IMGT) | of 411B08 using | | |
| | | IMGT | | |
| 53 | 411B08- | Amino acid | | IKEDGSEK |
| | CDRH2 | sequence of CDRH2 | | |
| | (IMGT) | of 411B08 using | | |
| | | IMGT | | |
| 54 | 411B08- | Amino acid | | ARNRLYSDFLDN |
| | CDRH3 | sequence of CDRH3 | | |
| | (IMGT) | of 411B08 using | | |
| | | IMGT | | |
| 55 | 411B08- | Amino acid | | SYWMS |
| | CDRH1 | sequence of CDRH1 | | |
| | (Kabat) | of 411B08 using | | |
| | | Kabat | | |
| 56 | 411B08- | Amino acid | | NIKEDGSEKYYVDSVKG |
| | CDRH2 | sequence of CDRH2 | | |
| | (Kabat) | of 411B08 using | | |
| | | Kabat | | |
| 57 | 411B08- | Amino acid | | NRLYSDFLDN |
| | CDRH3 | sequence of CDRH3 | | |
| | (Kabat) | of 411B08 using | | |
| | | Kabat | | |
| 58 | 411B08- | Amino acid | | EVQLVESGGGLVQPGGSLRLSCAASGFTFSSY |
| | Heavy | sequence of V_{H} of | | WMSWVRQAPGKGLEWVANIKEDGSEKYYV |
| | chain | 411B08 | | DSVKGRFTISRDNAKNSLYLQMNSLRAEDTS |
| | variable | | | VYYCARNRLYSDFLDNWGQGTLVTVSS |
| | region | | | |
| 59 | 411B08- | Nucleic acid | | GAGGTGCAGCTGGTGGAGTCTGGGGGAGG |
| | Heavy | sequence of V_{H} of | | CTTGGTCCAGCCTGGGGGGTCCCTGAGACT |
| | chain | 411B08 | | CTCCTGTGCAGCCTCTGGATTCACGTTTAGT |
| | variable | | | AGCTATTGGATGAGTTGGGTCCGCCAGGCT |
| | region | | | CCAGGGAAGGGGCTGGAGTGGGTGGCCAA |
| | | | | CATCAAAGAAGATGGAAGTGAGAAATACT |
| | | | | ATGTCGACTCTGTGAAGGGCCGATTCACCA |
| | | | | TCTCCAGAGACAACGCCAAGAACTCACTGT |
| | | | | ATCTGCAAATGAACAGCCTGAGAGCCGAGG |
| | | | | ACACGTCTGTGTATTACTGTGCGAGAAATC |
| | | | | GACTCTACAGTGACTTCCTTGACAACTGGG |
| | | | | GCCAGGGAACCCTGGTCACCGTCTCCTCAG |
| 60 | 411B08- | Amino acid | | EVQLVESGGGLVQPGGSLRLSCAASGFTFSSY |
| | full | sequence of 411B08 | | WMSWVRQAPGKGLEWVANIKEDGSEKYYV |
| | heavy | heavy chain | | DSVKGRFTISRDNAKNSLYLQMNSLRAEDTS |
| | chain | | | VYYCARNRLYSDFLDNWGQGTLVTVSSAST |
| | sequence | | | KGPSVFPLAPSSKSTSGGTAALGCLVKDYFPE |
| | | | | PVTVSWNSGALTSGVHTFPAVLQSSGLYSLSS |
| | | | | VVTVPSSSLGTQTYICNVNHKPSNTKVDKKV |
| | | | | EPKSCDKTHTCPPCPAPELLGGPSVFLFPPKPK |
| | | | | DTLMISRTPEVTCVVVDVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYTLPPSRDELTKNQVSLTCLVKGF |
| | | | | YPSDIAVEWESNGQPENNYKTTPPVLDSDGSF |
| | | | | FLYSKLTVDKSRWQQGNVFSCSVMHEALHN |
| | | | | HYTQKSLSLSPGK |
| 61 | 411B08- | Nucleic acid | | GAGGTGCAGCTGGTGGAGTCTGGGGGAGG |
| | full | sequence of 411B08 | | CTTGGTCCAGCCTGGGGGGTCCCTGAGACT |
| | heavy | heavy chain | | CTCCTGTGCAGCCTCTGGATTCACGTTTAGT |
| | chain | | | AGCTATTGGATGAGTTGGGTCCGCCAGGCT |
| | sequence | | | CCAGGGAAGGGGCTGGAGTGGGTGGCCAA |
| | | | | CATCAAAGAAGATGGAAGTGAGAAATACT |
| | | | | ATGTCGACTCTGTGAAGGGCCGATTCACCA |
| | | | | TCTCCAGAGACAACGCCAAGAACTCACTGT |
| | | | | ATCTGCAAATGAACAGCCTGAGAGCCGAGG |
| | | | | ACACGTCTGTGTATTACTGTGCGAGAAATC |
| | | | | GACTCTACAGTGACTTCCTTGACAACTGGG |
| | | | | GCCAGGGAACCCTGGTCACCGTCTCCTCAG |
| | | | | CCAGCACCAAGGGCCCCTCTGTGTTCCCTCT |
| | | | | GGCCCCTTCCAGCAAGTCCACCTCTGGCGG |
| | | | | AACAGCCGCTCTGGGCTGCCTCGTGAAGGA |
| | | | | CTACTTCCCCGAGCCTGTGACCGTGTCCTGG |
| | | | | AACTCTGGCGCTCTGACCAGCGGAGTGCAC |
| | | | | ACCTTCCCTGCTGTGCTGCAGTCCTCCGGCC |
| | | | | TGTACTCCCTGTCCTCCGTCGTGACCGTGCC |
| | | | | TTCCAGCTCTCTGGGCACCCAGACCTACAT |
| | | | | CTGCAACGTGAACCACAAGCCCTCCAACAC |
| | | | | CAAGGTGGACAAGAAGGTGGAACCCAAGT |
| | | | | CCTGCGACAAGACCCACACCTGTCCCCCTT |
| | | | | GTCCTGCCCCTGAACTGCTGGGCGGACCTT |
| | | | | CCGTGTTCCTGTTCCCCCCAAAGCCCAAGG |
| | | | | ACACCCTGATGATCTCCCGGACCCCCGAAG |
| | | | | TGACCTGCGTGGTGGTGGATGTGTCCCACG |
| | | | | AGGACCCTGAAGTGAAGTTCAATTGGTACG |
| | | | | TGGACGGCGTGGAAGTGCACAACGCCAAG |
| | | | | ACCAAGCCTAGAGAGGAACAGTACAACTCC |
| | | | | ACCTACCGGGTGGTGTCCGTGCTGACCGTG |
| | | | | CTGCACCAGGATTGGCTGAACGGCAAAGAG |
| | | | | TACAAGTGCAAGGTGTCCAACAAGGCCCTG |
| | | | | CCTGCCCCCATCGAAAAGACCATCTCCAAG |
| | | | | GCCAAGGGCCAGCCCCGGGAACCCCAGGT |
| | | | | GTACACACTGCCCCCTAGCAGGGACGAGCT |
| | | | | GACCAAGAACCAGGTGTCCCTGACCTGTCT |
| | | | | CGTGAAAGGCTTCTACCCCTCCGATATCGC |
| | | | | CGTGGAATGGGAGTCCAACGGCCAGCCTGA |
| | | | | GAACAACTACAAGACCACCCCCCCTGTGCT |
| | | | | GGACTCCGACGGCTCATTCTTCCTGTACAG |
| | | | | CAAGCTGACAGTGGACAAGTCCCGGTGGCA |
| | | | | GCAGGGCAACGTGTTCTCCTGCTCCGTGAT |
| | | | | GCACGAGGCCCTGCACAACCACTACACCCA |
| | | | | GAAGTCCCTGTCCCTGAGCCCCGGCAAG |
| 62 | 411B08- | Amino acid | | QGVSSW |
| | CDRL1 | sequence of CDRL1 | | |
| | (IMGT) | of 411B08 using | | |
| | | IMGT | | |
| 63 | 411B08- | Amino acid | | GAS |
| | CDRL2 | sequence of CDRL2 | | |
| | (IMGT) | of 411B08 using | | |
| | | IMGT | | |
| 64 | 411B08- | Amino acid | | QQANSIPFT |
| | CDRL3 | sequence of CDRL3 | | |
| | (IMGT) | of 411B08 using | | |
| | | IMGT | | |
| 65 | 411B08- | Amino acid | | RASQGVSSWLA |
| | CDRL1 | sequence of CDRL1 | | |
| | (Kabat) | of 411B08 using | | |
| | | Kabat | | |
| 66 | 411B08- | Amino acid | | GASSLQS |
| | CDRL2 | sequence of CDRL2 | | |
| | (Kabat) | of 411B08 using | | |
| | | Kabat | | |
| 67 | 411B08- | Amino acid | | QQANSIPFT |
| | CDRL3 | sequence of CDRL3 | | |
| | (Kabat) | of 411B08 using | | |
| | | Kabat | | |
| 68 | 411B08- | Amino acid | | DIQMTQSPSSVSASVGDRVTITCRASQGVSSW |
| | Light | sequence of V_{L} of | | LAWYQQKSGKAPKLLIYGASSLQSGVPSRFS |
| | chain | 411B08 | | GSGSGTEFILTISSLQPEDFATYYCQQANSIPFT |
| | variable | | | FGPGTKVDIK |
| | region | | | |
| 69 | 411B08- | Nucleic acid | | GACATCCAGATGACCCAGTCTCCATCTTCC |
| | Light | sequence of V_{L} of | | GTGTCTGCATCTGTCGGAGACAGAGTCACC |
| | chain | 411B08 | | ATCACTTGTCGGGCGAGTCAGGGTGTTAGC |
| | variable | | | AGCTGGTTAGCCTGGTATCAGCAGAAATCA |
| | region | | | GGGAAAGCCCCTAAGCTCCTGATCTATGGT |
| | | | | GCATCCAGTTTGCAAAGTGGGGTCCCATCA |
| | | | | AGATTCAGCGGCAGTGGATCTGGGACAGAG |
| | | | | TTCATTCTCACCATCAGCAGCCTGCAGCCTG |
| | | | | AAGATTTTGCAACTTACTATTGTCAACAGG |
| | | | | CTAACAGTATCCCATTCACTTTCGGCCCTGG |
| | | | | GACCAAAGTGGATATCAAAC |
| 70 | 411B08- | Amino acid | | DIQMTQSPSSVSASVGDRVTITCRASQGVSSW |
| | full | sequence of 411B08 | | LAWYQQKSGKAPKLLIYGASSLQSGVPSRFS |
| | light | light chain | | GSGSGTEFILTISSLQPEDFATYYCQQANSIPFT |
| | chain | | | FGPGTKVDIKRTVAAPSVFIFPPSDEQLKSGTA |
| | sequence | | | SVVCLLNNFYPREAKVQWKVDNALQSGNSQ |
| | | | | ESVTEQDSKDSTYSLSSTLTLSKADYEKHKVY |
| | | | | ACEVTHQGLSSPVTKSFNRGEC |
| 71 | 411B08- | Nucleic acid | | GACATCCAGATGACCCAGTCTCCATCTTCC |
| | full | sequence of 411B08 | | GTGTCTGCATCTGTCGGAGACAGAGTCACC |
| | light | light chain | | ATCACTTGTCGGGCGAGTCAGGGTGTTAGC |
| | chain | | | AGCTGGTTAGCCTGGTATCAGCAGAAATCA |
| | sequence | | | GGGAAAGCCCCTAAGCTCCTGATCTATGGT |
| | | | | GCATCCAGTTTGCAAAGTGGGGTCCCATCA |
| | | | | AGATTCAGCGGCAGTGGATCTGGGACAGAG |
| | | | | TTCATTCTCACCATCAGCAGCCTGCAGCCTG |
| | | | | AAGATTTTGCAACTTACTATTGTCAACAGG |
| | | | | CTAACAGTATCCCATTCACTTTCGGCCCTGG |
| | | | | GACCAAAGTGGATATCAAACGTACGGTGGC |
| | | | | CGCTCCCTCCGTGTTCATCTTCCCACCTTCC |
| | | | | GACGAGCAGCTGAAGTCCGGCACCGCTTCT |
| | | | | GTCGTGTGCCTGCTGAACAACTTCTACCCCC |
| | | | | GCGAGGCCAAGGTGCAGTGGAAGGTGGAC |
| | | | | AACGCCCTGCAGTCCGGCAACTCCCAGGAA |
| | | | | TCCGTGACCGAGCAGGACTCCAAGGACAGC |
| | | | | ACCTACTCCCTGTCCTCCACCCTGACCCTGT |
| | | | | CCAAGGCCGACTACGAGAAGCACAAGGTG |
| | | | | TACGCCTGCGAAGTGACCCACCAGGGCCTG |
| | | | | TCTAGCCCCGTGACCAAGTCTTTCAACCGG |
| | | | | GGCGAGTGT |
| 72 | 411C04- | Amino acid | | GFTFSSYW |
| | CDRH1 | sequence of CDRH1 | | |
| | (IMGT) | of 411C04 using | | |
| | | IMGT | | |
| 73 | 411C04- | Amino acid | | IKEDGSEK |
| | CDRH2 | sequence of CDRH2 | | |
| | (IMGT) | of 411C04 using | | |
| | | IMGT | | |
| 74 | 411C04- | Amino acid | | ARVRLYSDFLDY |
| | CDRH3 | sequence of CDRH3 | | |
| | (IMGT) | of 411C04 using | | |
| | | IMGT | | |
| 75 | 411C04- | Amino acid | | SYWMS |
| | CDRH1 | sequence of CDRH1 | | |
| | (Kabat) | of 411C04 using | | |
| | | Kabat | | |
| 76 | 411C04- | Amino acid | | NIKEDGSEKYYVDSLKG |
| | CDRH2 | sequence of CDRH2 | | |
| | (Kabat) | of 411C04 using | | |
| | | Kabat | | |
| 77 | 411C04- | Amino acid | | VRLYSDFLDY |
| | CDRH3 | sequence of CDRH3 | | |
| | (Kabat) | of 411C04 using | | |
| | | Kabat | | |
| 78 | 411C04- | Amino acid | | EVQLVDSGGGLVQPGGSLRLSCAASGFTFSSY |
| | Heavy | sequence of V_{H} of | | WMSWVRQAPGKGLEWVANIKEDGSEKYYV |
| | chain | 411C04 | | DSLKGRFTISRDNAKNSLYLQMNSLRAEDTS |
| | variable | | | VYYCARVRLYSDFLDYWGQGTLVTVSS |
| | region | | | |
| 79 | 411C04- | Nucleic acid | | GAGGTGCAGCTGGTGGACTCTGGGGGAGGC |
| | Heavy | sequence of V_{H} of | | TTGGTCCAGCCTGGGGGGTCCCTGAGACTC |
| | chain | 411C04 | | TCCTGTGCAGCCTCTGGATTCACGTTTAGTA |
| | variable | | | GCTATTGGATGAGTTGGGTCCGCCAGGCTC |
| | region | | | CAGGAAAGGGGCTGGAGTGGGTGGCCAAC |
| | | | | ATAAAAGAAGATGGAAGTGAGAAATACTA |
| | | | | TGTAGACTCTTTGAAGGGCCGATTCACCAT |
| | | | | CTCCAGAGACAACGCCAAGAACTCACTGTA |
| | | | | TCTGCAAATGAACAGCCTGAGAGCCGAGGA |
| | | | | CACGTCTGTGTATTACTGTGCGAGAGTTCG |
| | | | | ACTCTACAGTGACTTCCTTGACTACTGGGG |
| | | | | CCAGGGAACCCTGGTCACCGTCTCCTCAG |
| 80 | 411C04- | Amino acid | | EVQLVDSGGGLVQPGGSLRLSCAASGFTFSSY |
| | full | sequence of 411C04 | | WMSWVRQAPGKGLEWVANIKEDGSEKYYV |
| | heavy | heavy chain | | DSLKGRFTISRDNAKNSLYLQMNSLRAEDTS |
| | chain | | | VYYCARVRLYSDFLDYWGQGTLVTVSSAST |
| | sequence | | | KGPSVFPLAPSSKSTSGGTAALGCLVKDYFPE |
| | | | | PVTVSWNSGALTSGVHTFPAVLQSSGLYSLSS |
| | | | | VVTVPSSSLGTQTYICNVNHKPSNTKVDKKV |
| | | | | EPKSCDKTHTCPPCPAPELLGGPSVFLFPPKPK |
| | | | | DTLMISRTPEVTCVVVDVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYTLPPSRDELTKNQVSLTCLVKGF |
| | | | | YPSDIAVEWESNGQPENNYKTTPPVLDSDGSF |
| | | | | FLYSKLTVDKSRWQQGNVFSCSVMHEALHN |
| | | | | HYTQKSLSLSPGK |
| 81 | 411C04- | Nucleic acid | | GAGGTGCAGCTGGTGGACTCTGGGGGAGGC |
| | full | sequence of 411C04 | | TTGGTCCAGCCTGGGGGGTCCCTGAGACTC |
| | heavy | heavy chain | | TCCTGTGCAGCCTCTGGATTCACGTTTAGTA |
| | chain | | | GCTATTGGATGAGTTGGGTCCGCCAGGCTC |
| | sequence | | | CAGGAAAGGGGCTGGAGTGGGTGGCCAAC |
| | | | | ATAAAAGAAGATGGAAGTGAGAAATACTA |
| | | | | TGTAGACTCTTTGAAGGGCCGATTCACCAT |
| | | | | CTCCAGAGACAACGCCAAGAACTCACTGTA |
| | | | | TCTGCAAATGAACAGCCTGAGAGCCGAGGA |
| | | | | CACGTCTGTGTATTACTGTGCGAGAGTTCG |
| | | | | ACTCTACAGTGACTTCCTTGACTACTGGGG |
| | | | | CCAGGGAACCCTGGTCACCGTCTCCTCAGC |
| | | | | CAGCACCAAGGGCCCCTCTGTGTTCCCTCT |
| | | | | GGCCCCTTCCAGCAAGTCCACCTCTGGCGG |
| | | | | AACAGCCGCTCTGGGCTGCCTCGTGAAGGA |
| | | | | CTACTTCCCCGAGCCTGTGACCGTGTCCTGG |
| | | | | AACTCTGGCGCTCTGACCAGCGGAGTGCAC |
| | | | | ACCTTCCCTGCTGTGCTGCAGTCCTCCGGCC |
| | | | | TGTACTCCCTGTCCTCCGTCGTGACCGTGCC |
| | | | | TTCCAGCTCTCTGGGCACCCAGACCTACAT |
| | | | | CTGCAACGTGAACCACAAGCCCTCCAACAC |
| | | | | CAAGGTGGACAAGAAGGTGGAACCCAAGT |
| | | | | CCTGCGACAAGACCCACACCTGTCCCCCTT |
| | | | | GTCCTGCCCCTGAACTGCTGGGCGGACCTT |
| | | | | CCGTGTTCCTGTTCCCCCCAAAGCCCAAGG |
| | | | | ACACCCTGATGATCTCCCGGACCCCCGAAG |
| | | | | TGACCTGCGTGGTGGTGGATGTGTCCCACG |
| | | | | AGGACCCTGAAGTGAAGTTCAATTGGTACG |
| | | | | TGGACGGCGTGGAAGTGCACAACGCCAAG |
| | | | | ACCAAGCCTAGAGAGGAACAGTACAACTCC |
| | | | | ACCTACCGGGTGGTGTCCGTGCTGACCGTG |
| | | | | CTGCACCAGGATTGGCTGAACGGCAAAGAG |
| | | | | TACAAGTGCAAGGTGTCCAACAAGGCCCTG |
| | | | | CCTGCCCCCATCGAAAAGACCATCTCCAAG |
| | | | | GCCAAGGGCCAGCCCCGGGAACCCCAGGT |
| | | | | GTACACACTGCCCCCTAGCAGGGACGAGCT |
| | | | | GACCAAGAACCAGGTGTCCCTGACCTGTCT |
| | | | | CGTGAAAGGCTTCTACCCCTCCGATATCGC |
| | | | | CGTGGAATGGGAGTCCAACGGCCAGCCTGA |
| | | | | GAACAACTACAAGACCACCCCCCCTGTGCT |
| | | | | GGACTCCGACGGCTCATTCTTCCTGTACAG |
| | | | | CAAGCTGACAGTGGACAAGTCCCGGTGGCA |
| | | | | GCAGGGCAACGTGTTCTCCTGCTCCGTGAT |
| | | | | GCACGAGGCCCTGCACAACCACTACACCCA |
| | | | | GAAGTCCCTGTCCCTGAGCCCCGGCAAG |
| 82 | 411C04- | Amino acid | | QGVSSW |
| | CDRL1 | sequence of CDRL1 | | |
| | (IMGT) | of 411C04 using | | |
| | | IMGT | | |
| 83 | 411C04- | Amino acid | | GAS |
| | CDRL2 | sequence of CDRL2 | | |
| | (IMGT) | of 411C04 using | | |
| | | IMGT | | |
| 84 | 411C04- | Amino acid | | QQANSIPFT |
| | CDRL3 | sequence of CDRL3 | | |
| | (IMGT) | of 411C04 using | | |
| | | IMGT | | |
| 85 | 411C04- | Amino acid | | RASQGVSSWLA |
| | CDRL1 | sequence of CDRL1 | | |
| | (Kabat) | of 411C04 using | | |
| | | Kabat | | |
| 86 | 411C04- | Amino acid | | GASSLQS |
| | CDRL2 | sequence of CDRL2 | | |
| | (Kabat) | of 411C04 using | | |
| | | Kabat | | |
| 87 | 411C04- | Amino acid | | QQANSIPFT |
| | CDRL3 | sequence of CDRL3 | | |
| | (Kabat) | of 411C04 using | | |
| | | Kabat | | |
| 88 | 411C04- | Amino acid | | DIQMTQSPSSVSASVGDRVTITCRASQGVSSW |
| | Light | sequence of V_{L} of | | LAWYQQKSGKAPKLLIYGASSLQSGVPSRFS |
| | chain | 411C04 | | GSGSGTEFILSISSLQPEDFATYYCQQANSIPFT |
| | variable | | | FGPGTKVDIK |
| | region | | | |
| 89 | 411C04- | Nucleic acid | | GACATCCAGATGACCCAGTCTCCATCTTCC |
| | Light | sequence of V_{L} of | | GTGTCTGCATCTGTCGGAGACAGAGTCACC |
| | chain | 411C04 | | ATCACTTGTCGGGCGAGTCAGGGTGTTAGC |
| | variable | | | AGTTGGTTAGCCTGGTATCAGCAGAAATCA |
| | region | | | GGGAAAGCCCCTAAGCTCCTGATCTATGGT |
| | | | | GCCTCCAGTTTGCAAAGTGGGGTCCCATCA |
| | | | | AGATTCAGCGGCAGTGGATCTGGGACAGAG |
| | | | | TTCATTCTCAGCATCAGCAGCCTGCAGCCT |
| | | | | GAAGATTTTGCAACTTACTATTGTCAACAG |
| | | | | GCTAACAGTATCCCATTCACTTTCGGCCCTG |
| | | | | GGACCAAAGTGGATATCAAAC |
| 90 | 411C04- | Amino acid | | DIQMTQSPSSVSASVGDRVTITCRASQGVSSW |
| | full | sequence of 411C04 | | LAWYQQKSGKAPKLLIYGASSLQSGVPSRFS |
| | light | light chain | | GSGSGTEFILSISSLQPEDFATYYCQQANSIPFT |
| | chain | | | FGPGTKVDIKRTVAAPSVFIFPPSDEQLKSGTA |
| | sequence | | | SVVCLLNNFYPREAKVQWKVDNALQSGNSQ |
| | | | | ESVTEQDSKDSTYSLSSTLTLSKADYEKHKVY |
| | | | | ACEVTHQGLSSPVTKSFNRGEC |
| 91 | 411C04- | Nucleic acid | | GACATCCAGATGACCCAGTCTCCATCTTCC |
| | full | sequence of 411C04 | | GTGTCTGCATCTGTCGGAGACAGAGTCACC |
| | light | light chain | | ATCACTTGTCGGGCGAGTCAGGGTGTTAGC |
| | chain | | | AGTTGGTTAGCCTGGTATCAGCAGAAATCA |
| | sequence | | | GGGAAAGCCCCTAAGCTCCTGATCTATGGT |
| | | | | GCCTCCAGTTTGCAAAGTGGGGTCCCATCA |
| | | | | AGATTCAGCGGCAGTGGATCTGGGACAGAG |
| | | | | TTCATTCTCAGCATCAGCAGCCTGCAGCCT |
| | | | | GAAGATTTTGCAACTTACTATTGTCAACAG |
| | | | | GCTAACAGTATCCCATTCACTTTCGGCCCTG |
| | | | | GGACCAAAGTGGATATCAAACGTACGGTGG |
| | | | | CCGCTCCCTCCGTGTTCATCTTCCCACCTTC |
| | | | | CGACGAGCAGCTGAAGTCCGGCACCGCTTC |
| | | | | TGTCGTGTGCCTGCTGAACAACTTCTACCCC |
| | | | | CGCGAGGCCAAGGTGCAGTGGAAGGTGGA |
| | | | | CAACGCCCTGCAGTCCGGCAACTCCCAGGA |
| | | | | ATCCGTGACCGAGCAGGACTCCAAGGACAG |
| | | | | CACCTACTCCCTGTCCTCCACCCTGACCCTG |
| | | | | TCCAAGGCCGACTACGAGAAGCACAAGGT |
| | | | | GTACGCCTGCGAAGTGACCCACCAGGGCCT |
| | | | | GTCTAGCCCCGTGACCAAGTCTTTCAACCG |
| | | | | GGGCGAGTGT |
| 92 | 411D07- | Amino acid | | GGSIISSDW |
| | CDRH1 | sequence of CDRH1 | | |
| | (IMGT) | of 411D07 using | | |
| | | IMGT | | |
| 93 | 411D07- | Amino acid | | IFHSGRT |
| | CDRH2 | sequence of CDRH2 | | |
| | (IMGT) | of 411D07 using | | |
| | | IMGT | | |
| 94 | 411D07- | Amino acid | | ARDGSGSY |
| | CDRH3 | sequence of CDRH3 | | |
| | (IMGT) | of 411D07 using | | |
| | | IMGT | | |
| 95 | 411D07- | Amino acid | | SSDWWN |
| | CDRH1 | sequence of CDRH1 | | |
| | (Kabat) | of 411D07 using | | |
| | | Kabat | | |
| 96 | 411D07- | Amino acid | | EIFHSGRTNYNPSLKS |
| | CDRH2 | sequence of CDRH2 | | |
| | (Kabat) | of 411D07 using | | |
| | | Kabat | | |
| 97 | 411D07- | Amino acid | | DGSGSY |
| | CDRH3 | sequence of CDRH3 | | |
| | (Kabat) | of 411D07 using | | |
| | | Kabat | | |
| 98 | 411D07- | Amino acid | | QVQLQESGPGLVKPSGTLSLTCIVSGGSIISSD |
| | Heavy | sequence of V_{H} of | | WWNWVRQPPGKGLEWIGEIFHSGRTNYNPSL |
| | chain | 411D07 | | KSRVTISIDKSKNQFSLRLSSVTAADTAVYYC |
| | variable | | | ARDGSGSYWGQGTLVTVSS |
| | region | | | |
| 99 | 411D07- | Nucleic acid | | CAGGTGCAGCTGCAGGAGTCGGGCCCAGG |
| | Heavy | sequence of V_{H} of | | ACTGGTGAAGCCTTCGGGGACCCTGTCCCT |
| | chain | 411D07 | | CACCTGCATTGTCTCTGGTGGCTCCATCATC |
| | variable | | | AGTAGTGACTGGTGGAATTGGGTCCGCCAG |
| | region | | | CCCCCAGGGAAGGGGCTGGAGTGGATTGG |
| | | | | AGAAATCTTTCATAGTGGGAGGACCAACTA |
| | | | | CAACCCGTCCCTCAAGAGTCGAGTCACCAT |
| | | | | ATCAATAGACAAGTCCAAGAATCAGTTCTC |
| | | | | CCTGAGGCTGAGCTCTGTGACCGCCGCGGA |
| | | | | CACGGCCGTGTATTACTGTGCGAGAGATGG |
| | | | | TTCGGGGAGTTACTGGGGCCAGGGAACCCT |
| | | | | GGTCACCGTCTCCTCAG |
| 100 | 411D07- | Amino acid | | QVQLQESGPGLVKPSGTLSLTCIVSGGSIISSD |
| | full | sequence of 411D07 | | WWNWVRQPPGKGLEWIGEIFHSGRTNYNPSL |
| | heavy | heavy chain | | KSRVTISIDKSKNQFSLRLSSVTAADTAVYYC |
| | chain | | | ARDGSGSYWGQGTLVTVSSASTKGPSVFPLA |
| | sequence | | | PSSKSTSGGTAALGCLVKDYFPEPVTVSWNS |
| | | | | GALTSGVHTFPAVLQSSGLYSLSSVVTVPSSS |
| | | | | LGTQTYICNVNHKPSNTKVDKKVEPKSCDKT |
| | | | | HTCPPCPAPELLGGPSVFLFPPKPKDTLMISRT |
| | | | | PEVTCVVVDVSHEDPEVKFNWYVDGVEVHN |
| | | | | AKTKPREEQYNSTYRVVSVLTVLHQDWLNG |
| | | | | KEYKCKVSNKALPAPIEKTISKAKGQPREPQV |
| | | | | YTLPPSRDELTKNQVSLTCLVKGFYPSDIAVE |
| | | | | WESNGQPENNYKTTPPVLDSDGSFFLYSKLT |
| | | | | VDKSRWQQGNVFSCSVMHEALHNHYTQKSL |
| | | | | SLSPGK |
| 101 | 411D07- | Nucleic acid | | CAGGTGCAGCTGCAGGAGTCGGGCCCAGG |
| | full | sequence of 411D07 | | ACTGGTGAAGCCTTCGGGGACCCTGTCCCT |
| | heavy | heavy chain | | CACCTGCATTGTCTCTGGTGGCTCCATCATC |
| | chain | | | AGTAGTGACTGGTGGAATTGGGTCCGCCAG |
| | sequence | | | CCCCCAGGGAAGGGGCTGGAGTGGATTGG |
| | | | | AGAAATCTTTCATAGTGGGAGGACCAACTA |
| | | | | CAACCCGTCCCTCAAGAGTCGAGTCACCAT |
| | | | | ATCAATAGACAAGTCCAAGAATCAGTTCTC |
| | | | | CCTGAGGCTGAGCTCTGTGACCGCCGCGGA |
| | | | | CACGGCCGTGTATTACTGTGCGAGAGATGG |
| | | | | TTCGGGGAGTTACTGGGGCCAGGGAACCCT |
| | | | | GGTCACCGTCTCCTCAGCCAGCACCAAGGG |
| | | | | CCCCTCTGTGTTCCCTCTGGCCCCTTCCAGC |
| | | | | AAGTCCACCTCTGGCGGAACAGCCGCTCTG |
| | | | | GGCTGCCTCGTGAAGGACTACTTCCCCGAG |
| | | | | CCTGTGACCGTGTCCTGGAACTCTGGCGCT |
| | | | | CTGACCAGCGGAGTGCACACCTTCCCTGCT |
| | | | | GTGCTGCAGTCCTCCGGCCTGTACTCCCTGT |
| | | | | CCTCCGTCGTGACCGTGCCTTCCAGCTCTCT |
| | | | | GGGCACCCAGACCTACATCTGCAACGTGAA |
| | | | | CCACAAGCCCTCCAACACCAAGGTGGACAA |
| | | | | GAAGGTGGAACCCAAGTCCTGCGACAAGA |
| | | | | CCCACACCTGTCCCCCTTGTCCTGCCCCTGA |
| | | | | ACTGCTGGGCGGACCTTCCGTGTTCCTGTTC |
| | | | | CCCCCAAAGCCCAAGGACACCCTGATGATC |
| | | | | TCCCGGACCCCCGAAGTGACCTGCGTGGTG |
| | | | | GTGGATGTGTCCCACGAGGACCCTGAAGTG |
| | | | | AAGTTCAATTGGTACGTGGACGGCGTGGAA |
| | | | | GTGCACAACGCCAAGACCAAGCCTAGAGA |
| | | | | GGAACAGTACAACTCCACCTACCGGGTGGT |
| | | | | GTCCGTGCTGACCGTGCTGCACCAGGATTG |
| | | | | GCTGAACGGCAAAGAGTACAAGTGCAAGG |
| | | | | TGTCCAACAAGGCCCTGCCTGCCCCCATCG |
| | | | | AAAAGACCATCTCCAAGGCCAAGGGCCAG |
| | | | | CCCCGGGAACCCCAGGTGTACACACTGCCC |
| | | | | CCTAGCAGGGACGAGCTGACCAAGAACCA |
| | | | | GGTGTCCCTGACCTGTCTCGTGAAAGGCTT |
| | | | | CTACCCCTCCGATATCGCCGTGGAATGGGA |
| | | | | GTCCAACGGCCAGCCTGAGAACAACTACAA |
| | | | | GACCACCCCCCCTGTGCTGGACTCCGACGG |
| | | | | CTCATTCTTCCTGTACAGCAAGCTGACAGT |
| | | | | GGACAAGTCCCGGTGGCAGCAGGGCAACG |
| | | | | TGTTCTCCTGCTCCGTGATGCACGAGGCCCT |
| | | | | GCACAACCACTACACCCAGAAGTCCCTGTC |
| | | | | CCTGAGCCCCGGCAAG |
| 102 | 411D07- | Amino acid | | QSVLYSSNNKNY |
| | CDRL1 | sequence of CDRL1 | | |
| | (IMGT) | of 411D07 using | | |
| | | IMGT | | |
| 103 | 411D07- | Amino acid | | WAS |
| | CDRL2 | sequence of CDRL2 | | |
| | (IMGT) | of 411D07 using | | |
| | | IMGT | | |
| 104 | 411D07- | Amino acid | | QQYYSNRS |
| | CDRL3 | sequence of CDRL3 | | |
| | (IMGT) | of 411D07 using | | |
| | | IMGT | | |
| 105 | 411D07- | Amino acid | | KSSQSVLYSSNNKNYLA |
| | CDRL1 | sequence of CDRL1 | | |
| | (Kabat) | of 411D07 using | | |
| | | Kabat | | |
| 106 | 411D07- | Amino acid | | WASTRES |
| | CDRL2 | sequence of CDRL2 | | |
| | (Kabat) | of 411D07 using | | |
| | | Kabat | | |
| 107 | 411D07- | Amino acid | | QQYYSNRS |
| | CDRL3 | sequence of CDRL3 | | |
| | (Kabat) | of 411D07 using | | |
| | | Kabat | | |
| 108 | 411D07- | Amino acid | | DIVMTQSPDSLAVSLGERATINCKSSQSVLYS |
| | Light | sequence of V_{L} of | | SNNKNYLAWYQQKSGQPPKLLIYWASTRESG |
| | chain | 411D07 | | VPDRFSGSGSGTDFTLTISSLQTEDVAVYYCQ |
| | variable | | | QYYSNRSFGQGTKLEIK |
| | region | | | |
| 109 | 411D07- | Nucleic acid | | GACATCGTGATGACCCAGTCTCCAGACTCC |
| | Light | sequence of V_{L} of | | CTGGCTGTGTCTCTGGGCGAGAGGGCCACC |
| | chain | 411D07 | | ATCAACTGCAAGTCCAGCCAGAGTGTTTTA |
| | variable | | | TACAGCTCCAACAATAAGAATTACTTAGCT |
| | region | | | TGGTACCAGCAGAAATCAGGACAGCCTCCT |
| | | | | AAGTTGCTCATTTACTGGGCATCTACCCGG |
| | | | | GAATCCGGGGTCCCTGACCGATTCAGTGGC |
| | | | | AGCGGGTCTGGGACAGATTTCACTCTCACC |
| | | | | ATCAGCAGCCTGCAGACTGAAGATGTGGCA |
| | | | | gtttattactgtcagcaatattatagtaatc |
| | | | | GCAGTTTTGGCCAGGGGACCAAGCTGGAGA |
| | | | | TCAAAC |
| 110 | 411D07- | Amino acid | | DIVMTQSPDSLAVSLGERATINCKSSQSVLYS |
| | full | sequence of 411D07 | | SNNKNYLAWYQQKSGQPPKLLIYWASTRESG |
| | light | light chain | | VPDRFSGSGSGTDFTLTISSLQTEDVAVYYCQ |
| | chain | | | QYYSNRSFGQGTKLEIKRTVAAPSVFIFPPSDE |
| | sequence | | | QLKSGTASVVCLLNNFYPREAKVQWKVDNA |
| | | | | LQSGNSQESVTEQDSKDSTYSLSSTLTLSKAD |
| | | | | YEKHKVYACEVTHQGLSSPVTKSFNRGEC |
| 111 | 411D07- | Nucleic acid | | GACATCGTGATGACCCAGTCTCCAGACTCC |
| | full | sequence of 411D07 | | CTGGCTGTGTCTCTGGGCGAGAGGGCCACC |
| | light | light chain | | ATCAACTGCAAGTCCAGCCAGAGTGTTTTA |
| | chain | | | TACAGCTCCAACAATAAGAATTACTTAGCT |
| | sequence | | | TGGTACCAGCAGAAATCAGGACAGCCTCCT |
| | | | | AAGTTGCTCATTTACTGGGCATCTACCCGG |
| | | | | GAATCCGGGGTCCCTGACCGATTCAGTGGC |
| | | | | AGCGGGTCTGGGACAGATTTCACTCTCACC |
| | | | | ATCAGCAGCCTGCAGACTGAAGATGTGGCA |
| | | | | gtttattactgtcagcaatattatagtaatc |
| | | | | GCAGTTTTGGCCAGGGGACCAAGCTGGAGA |
| | | | | TCAAACGTACGGTGGCCGCTCCCTCCGTGT |
| | | | | TCATCTTCCCACCTTCCGACGAGCAGCTGA |
| | | | | AGTCCGGCACCGCTTCTGTCGTGTGCCTGCT |
| | | | | GAACAACTTCTACCCCCGCGAGGCCAAGGT |
| | | | | GCAGTGGAAGGTGGACAACGCCCTGCAGTC |
| | | | | CGGCAACTCCCAGGAATCCGTGACCGAGCA |
| | | | | GGACTCCAAGGACAGCACCTACTCCCTGTC |
| | | | | CTCCACCCTGACCCTGTCCAAGGCCGACTA |
| | | | | CGAGAAGCACAAGGTGTACGCCTGCGAAGT |
| | | | | GACCCACCAGGGCCTGTCTAGCCCCGTGAC |
| | | | | CAAGTCTTTCAACCGGGGCGAGTGT |
| 112 | 385F01- | Amino acid | | GFTFSSYW |
| | CDRH1 | sequence of CDRH1 | | |
| | (IMGT) | of 385F01 using | | |
| | | IMGT | | |
| 113 | 385F01- | Amino acid | | IKEDGSEK |
| | CDRH2 | sequence of CDRH2 | | |
| | (IMGT) | of 385F01 using | | |
| | | IMGT | | |
| 114 | 385F01- | Amino acid | | ARNRLYSDFLDN |
| | CDRH3 | sequence of CDRH3 | | |
| | (IMGT) | of 385F01 using | | |
| | | IMGT | | |
| 115 | 385F01- | Amino acid | | SYWMS |
| | CDRH1 | sequence of CDRH1 | | |
| | (Kabat) | of 385F01 using | | |
| | | Kabat | | |
| 116 | 385F01- | Amino acid | | NIKEDGSEKYYVDSVKG |
| | CDRH2 | sequence of CDRH2 | | |
| | (Kabat) | of 385F01 using | | |
| | | Kabat | | |
| 117 | 385F01- | Amino acid | | NRLYSDFLDN |
| | CDRH3 | sequence of CDRH3 | | |
| | (Kabat) | of 385F01 using | | |
| | | Kabat | | |
| 118 | 385F01- | Amino acid | | EVQLVESGGGLVQPGGSLRLSCAASGFTFSSY |
| | Heavy | sequence of V_{H} of | | WMSWVRQAPGKGLEWVANIKEDGSEKYYV |
| | chain | 385F01 | | DSVKGRFTISRDNAKNSLYLQMNSLRAEDTS |
| | variable | | | VYYCARNRLYSDFLDNWGQGTLVTVSS |
| | region | | | |
| 119 | 385F01- | Nucleic acid | | GAGGTGCAGCTGGTGGAGTCTGGGGGAGG |
| | Heavy | sequence of V_{H} of | | CTTGGTCCAGCCTGGGGGGTCCCTGAGACT |
| | chain | 385F01 | | CTCCTGTGCAGCCTCTGGATTCACGTTTAGT |
| | variable | | | AGCTATTGGATGAGTTGGGTCCGCCAGGCT |
| | region | | | CCAGGGAAGGGGCTGGAGTGGGTGGCCAA |
| | | | | CATCAAAGAAGATGGAAGTGAGAAATACT |
| | | | | ATGTCGACTCTGTGAAGGGCCGATTCACCA |
| | | | | TCTCCAGAGACAACGCCAAGAACTCACTGT |
| | | | | ATCTGCAAATGAACAGCCTGAGAGCCGAGG |
| | | | | ACACGTCTGTGTATTACTGTGCGAGAAATC |
| | | | | GACTCTACAGTGACTTCCTTGACAACTGGG |
| | | | | GCCAGGGAACCCTGGTCACCGTCTCCTCAG |
| 120 | 385F01- | Amino acid | | EVQLVESGGGLVQPGGSLRLSCAASGFTFSSY |
| | full | sequence of385F01 | | WMSWVRQAPGKGLEWVANIKEDGSEKYYV |
| | heavy | heavy chain | | DSVKGRFTISRDNAKNSLYLQMNSLRAEDTS |
| | chain | | | VYYCARNRLYSDFLDNWGQGTLVTVSSAST |
| | sequence | | | KGPSVFPLAPSSKSTSGGTAALGCLVKDYFPE |
| | | | | PVTVSWNSGALTSGVHTFPAVLQSSGLYSLSS |
| | | | | VVTVPSSSLGTQTYICNVNHKPSNTKVDKKV |
| | | | | EPKSCDKTHTCPPCPAPELLGGPSVFLFPPKPK |
| | | | | DTLMISRTPEVTCVVVDVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYTLPPSRDELTKNQVSLTCLVKGF |
| | | | | YPSDIAVEWESNGQPENNYKTTPPVLDSDGSF |
| | | | | FLYSKLTVDKSRWQQGNVFSCSVMHEALHN |
| | | | | HYTQKSLSLSPGK |
| 121 | 385F01- | Nucleic acid | | GAGGTGCAGCTGGTGGAGTCTGGGGGAGG |
| | full | sequence of385F01 | | CTTGGTCCAGCCTGGGGGGTCCCTGAGACT |
| | heavy | heavy chain | | CTCCTGTGCAGCCTCTGGATTCACGTTTAGT |
| | chain | | | AGCTATTGGATGAGTTGGGTCCGCCAGGCT |
| | sequence | | | CCAGGGAAGGGGCTGGAGTGGGTGGCCAA |
| | | | | CATCAAAGAAGATGGAAGTGAGAAATACT |
| | | | | ATGTCGACTCTGTGAAGGGCCGATTCACCA |
| | | | | TCTCCAGAGACAACGCCAAGAACTCACTGT |
| | | | | ATCTGCAAATGAACAGCCTGAGAGCCGAGG |
| | | | | ACACGTCTGTGTATTACTGTGCGAGAAATC |
| | | | | GACTCTACAGTGACTTCCTTGACAACTGGG |
| | | | | GCCAGGGAACCCTGGTCACCGTCTCCTCAG |
| | | | | CCAGCACCAAGGGCCCCTCTGTGTTCCCTCT |
| | | | | GGCCCCTTCCAGCAAGTCCACCTCTGGCGG |
| | | | | AACAGCCGCTCTGGGCTGCCTCGTGAAGGA |
| | | | | CTACTTCCCCGAGCCTGTGACCGTGTCCTGG |
| | | | | AACTCTGGCGCTCTGACCAGCGGAGTGCAC |
| | | | | ACCTTCCCTGCTGTGCTGCAGTCCTCCGGCC |
| | | | | TGTACTCCCTGTCCTCCGTCGTGACCGTGCC |
| | | | | TTCCAGCTCTCTGGGCACCCAGACCTACAT |
| | | | | CTGCAACGTGAACCACAAGCCCTCCAACAC |
| | | | | CAAGGTGGACAAGAAGGTGGAACCCAAGT |
| | | | | CCTGCGACAAGACCCACACCTGTCCCCCTT |
| | | | | GTCCTGCCCCTGAACTGCTGGGCGGACCTT |
| | | | | CCGTGTTCCTGTTCCCCCCAAAGCCCAAGG |
| | | | | ACACCCTGATGATCTCCCGGACCCCCGAAG |
| | | | | TGACCTGCGTGGTGGTGGATGTGTCCCACG |
| | | | | AGGACCCTGAAGTGAAGTTCAATTGGTACG |
| | | | | TGGACGGCGTGGAAGTGCACAACGCCAAG |
| | | | | ACCAAGCCTAGAGAGGAACAGTACAACTCC |
| | | | | ACCTACCGGGTGGTGTCCGTGCTGACCGTG |
| | | | | CTGCACCAGGATTGGCTGAACGGCAAAGAG |
| | | | | TACAAGTGCAAGGTGTCCAACAAGGCCCTG |
| | | | | CCTGCCCCCATCGAAAAGACCATCTCCAAG |
| | | | | GCCAAGGGCCAGCCCCGGGAACCCCAGGT |
| | | | | GTACACACTGCCCCCTAGCAGGGACGAGCT |
| | | | | GACCAAGAACCAGGTGTCCCTGACCTGTCT |
| | | | | CGTGAAAGGCTTCTACCCCTCCGATATCGC |
| | | | | CGTGGAATGGGAGTCCAACGGCCAGCCTGA |
| | | | | GAACAACTACAAGACCACCCCCCCTGTGCT |
| | | | | GGACTCCGACGGCTCATTCTTCCTGTACAG |
| | | | | CAAGCTGACAGTGGACAAGTCCCGGTGGCA |
| | | | | GCAGGGCAACGTGTTCTCCTGCTCCGTGAT |
| | | | | GCACGAGGCCCTGCACAACCACTACACCCA |
| | | | | GAAGTCCCTGTCCCTGAGCCCCGGCAAG |
| 122 | 385F01- | Amino acid | | QGVSSW |
| | CDRL1 | sequence of CDRL1 | | |
| | (IMGT) | of 385F01 using | | |
| | | IMGT | | |
| 123 | 385F01- | Amino acid | | GAS |
| | CDRL2 | sequence of CDRL2 | | |
| | (IMGT) | of 385F01 using | | |
| | | IMGT | | |
| 124 | 385F01- | Amino acid | | QQANSIPFT |
| | CDRL3 | sequence of CDRL3 | | |
| | (IMGT) | of 385F01 using | | |
| | | IMGT | | |
| 125 | 385F01- | Amino acid | | RASQGVSSWLA |
| | CDRL1 | sequence of CDRL1 | | |
| | (Kabat) | of 385F01 using | | |
| | | Kabat | | |
| 126 | 385F01- | Amino acid | | GASSLQS |
| | CDRL2 | sequence of CDRL2 | | |
| | (Kabat) | of 385F01 using | | |
| | | Kabat | | |
| 127 | 385F01- | Amino acid | | QQANSIPFT |
| | CDRL3 | sequence of CDRL3 | | |
| | (Kabat) | of 385F01 using | | |
| | | Kabat | | |
| 128 | 385F01- | Amino acid | | DIQMTQSPSSVSASVGDRVTITCRASQGVSSW |
| | Light | sequence of V_{L} of | | LAWYQQKSGKAPKLLIYGASSLQSGVPSRFS |
| | chain | 385F01 | | GSGSGTEFILTISSLQPEDFATYYCQQANSIPFT |
| | variable | | | FGPGTKVDIK |
| | region | | | |
| 129 | 385F01- | Nucleic acid | | GACATCCAGATGACCCAGTCTCCATCTTCC |
| | Light | sequence of V_{L} of | | GTGTCTGCATCTGTCGGAGACAGAGTCACC |
| | chain | 385F01 | | ATCACTTGTCGGGCGAGTCAGGGTGTTAGC |
| | variable | | | AGCTGGTTAGCCTGGTATCAGCAGAAATCA |
| | region | | | GGGAAAGCCCCTAAGCTCCTGATCTATGGT |
| | | | | GCATCCAGTTTGCAAAGTGGGGTCCCATCA |
| | | | | AGATTCAGCGGCAGTGGATCTGGGACAGAG |
| | | | | TTCATTCTCACCATCAGCAGCCTGCAGCCTG |
| | | | | AAGATTTTGCAACTTACTATTGTCAACAGG |
| | | | | CTAACAGTATCCCATTCACTTTCGGCCCTGG |
| | | | | GACCAAAGTGGATATCAAAC |
| 130 | 385F01- | Amino acid | | DIQMTQSPSSVSASVGDRVTITCRASQGVSSW |
| | full light | sequence of 385F01 | | LAWYQQKSGKAPKLLIYGASSLQSGVPSRFS |
| | chain | light chain | | GSGSGTEFILTISSLQPEDFATYYCQQANSIPFT |
| | sequence | | | FGPGTKVDIKRTVAAPSVFIFPPSDEQLKSGTA |
| | | | | SVVCLLNNFYPREAKVQWKVDNALQSGNSQ |
| | | | | ESVTEQDSKDSTYSLSSTLTLSKADYEKHKVY |
| | | | | ACEVTHQGLSSPVTKSFNRGEC |
| 131 | 385F01- | Nucleic acid | | GACATCCAGATGACCCAGTCTCCATCTTCC |
| | full light | sequence of 385F01 | | GTGTCTGCATCTGTCGGAGACAGAGTCACC |
| | chain | light chain | | ATCACTTGTCGGGCGAGTCAGGGTGTTAGC |
| | sequence | | | AGCTGGTTAGCCTGGTATCAGCAGAAATCA |
| | | | | GGGAAAGCCCCTAAGCTCCTGATCTATGGT |
| | | | | GCATCCAGTTTGCAAAGTGGGGTCCCATCA |
| | | | | AGATTCAGCGGCAGTGGATCTGGGACAGAG |
| | | | | TTCATTCTCACCATCAGCAGCCTGCAGCCTG |
| | | | | AAGATTTTGCAACTTACTATTGTCAACAGG |
| | | | | CTAACAGTATCCCATTCACTTTCGGCCCTGG |
| | | | | GACCAAAGTGGATATCAAACGTACGGTGGC |
| | | | | CGCTCCCTCCGTGTTCATCTTCCCACCTTCC |
| | | | | GACGAGCAGCTGAAGTCCGGCACCGCTTCT |
| | | | | GTCGTGTGCCTGCTGAACAACTTCTACCCCC |
| | | | | GCGAGGCCAAGGTGCAGTGGAAGGTGGAC |
| | | | | AACGCCCTGCAGTCCGGCAACTCCCAGGAA |
| | | | | TCCGTGACCGAGCAGGACTCCAAGGACAGC |
| | | | | ACCTACTCCCTGTCCTCCACCCTGACCCTGT |
| | | | | CCAAGGCCGACTACGAGAAGCACAAGGTG |
| | | | | TACGCCTGCGAAGTGACCCACCAGGGCCTG |
| | | | | TCTAGCCCCGTGACCAAGTCTTTCAACCGG |
| | | | | GGCGAGTGT |
| 132 | 413D08- | Amino acid | | GFTFRIYG |
| | CDRH1 | sequence of CDRH1 | | |
| | (IMGT) | of 413D08 using | | |
| | | IMGT | | |
| 133 | 413D08- | Amino acid | | IWYDGSNK |
| | CDRH2 | sequence of CDRH2 | | |
| | (IMGT) | of 413D08 using | | |
| | | IMGT | | |
| 134 | 413D08- | Amino acid | | ARDMDYFGMDV |
| | CDRH3 | sequence of CDRH3 | | |
| | (IMGT) | of 413D08 using | | |
| | | IMGT | | |
| 135 | 413D08- | Amino acid | | IYGMH |
| | CDRH1 | sequence of CDRH1 | | |
| | (Kabat) | of 413D08 using | | |
| | | Kabat | | |
| 136 | 413D08- | Amino acid | | VIWYDGSNKYYADSVKG |
| | CDRH2 | sequence of CDRH2 | | |
| | (Kabat) | of 413D08 using | | |
| | | Kabat | | |
| 137 | 413D08- | Amino acid | | DMDYFGMDV |
| | CDRH3 | sequence of CDRH3 | | |
| | (Kabat) | of 413D08 using | | |
| | | Kabat | | |
| 138 | 413D08- | Amino acid | | QVQLVESGGGVVQPGRSLRLSCAASGFTFRIY |
| | Heavy | sequence of V_{H} of | | GMHWVRQAPGKGLEWVAVIWYDGSNKYYA |
| | chain | 413D08 | | DSVKGRFTISRDNSDNTLYLQMNSLRAEDTA |
| | variable | | | VYYCARDMDYFGMDVWGQGTTVTVSS |
| | region | | | |
| 139 | 413D08- | Nucleic acid | | CAGGTGCAGCTGGTGGAGTCTGGGGGAGGC |
| | Heavy | sequence of V_{H} of | | GTGGTCCAGCCTGGGAGGTCCCTGAGACTC |
| | chain | 413D08 | | TCCTGTGCAGCGTCTGGATTCACCTTCCGTA |
| | variable | | | TTTATGGCATGCACTGGGTCCGCCAGGCTC |
| | region | | | CAGGCAAGGGGCTGGAGTGGGTGGCAGTT |
| | | | | ATATGGTATGATGGAAGTAATAAATACTAT |
| | | | | GCTGACTCCGTGAAGGGCCGATTCACCATC |
| | | | | TCCAGAGACAATTCCGACAACACGCTGTAT |
| | | | | CTGCAAATGAACAGCCTGAGAGCCGAGGA |
| | | | | CACGGCTGTGTATTACTGTGCGAGAGATAT |
| | | | | GGACTACTTCGGTATGGACGTCTGGGGCCA |
| | | | | AGGGACCACGGTCACCGTCTCCTCAG |
| 140 | 413D08- | Amino acid | | QVQLVESGGGVVQPGRSLRLSCAASGFTFRIY |
| | full | sequence of 413D08 | | GMHWVRQAPGKGLEWVAVIWYDGSNKYYA |
| | heavy | heavy chain | | DSVKGRFTISRDNSDNTLYLQMNSLRAEDTA |
| | chain | | | VYYCARDMDYFGMDVWGQGTTVTVSSAST |
| | sequence | | | KGPSVFPLAPSSKSTSGGTAALGCLVKDYFPE |
| | | | | PVTVSWNSGALTSGVHTFPAVLQSSGLYSLSS |
| | | | | VVTVPSSSLGTQTYICNVNHKPSNTKVDKKV |
| | | | | EPKSCDKTHTCPPCPAPELLGGPSVFLFPPKPK |
| | | | | DTLMISRTPEVTCVVVDVSHEDPEVKFNWYV |
| | | | | DGVEVHNAKTKPREEQYNSTYRVVSVLTVL |
| | | | | HQDWLNGKEYKCKVSNKALPAPIEKTISKAK |
| | | | | GQPREPQVYTLPPSRDELTKNQVSLTCLVKGF |
| | | | | YPSDIAVEWESNGQPENNYKTTPPVLDSDGSF |
| | | | | FLYSKLTVDKSRWQQGNVFSCSVMHEALHN |
| | | | | HYTQKSLSLSPGK |
| 141 | 413D08- | Nucleic acid | | CAGGTGCAGCTGGTGGAGTCTGGGGGAGGC |
| | full | sequence of 413D08 | | GTGGTCCAGCCTGGGAGGTCCCTGAGACTC |
| | heavy | heavy chain | | TCCTGTGCAGCGTCTGGATTCACCTTCCGTA |
| | chain | | | TTTATGGCATGCACTGGGTCCGCCAGGCTC |
| | sequence | | | CAGGCAAGGGGCTGGAGTGGGTGGCAGTT |
| | | | | ATATGGTATGATGGAAGTAATAAATACTAT |
| | | | | GCTGACTCCGTGAAGGGCCGATTCACCATC |
| | | | | TCCAGAGACAATTCCGACAACACGCTGTAT |
| | | | | CTGCAAATGAACAGCCTGAGAGCCGAGGA |
| | | | | CACGGCTGTGTATTACTGTGCGAGAGATAT |
| | | | | GGACTACTTCGGTATGGACGTCTGGGGCCA |
| | | | | AGGGACCACGGTCACCGTCTCCTCAGCCAG |
| | | | | CACCAAGGGCCCCTCTGTGTTCCCTCTGGCC |
| | | | | CCTTCCAGCAAGTCCACCTCTGGCGGAACA |
| | | | | GCCGCTCTGGGCTGCCTCGTGAAGGACTAC |
| | | | | TTCCCCGAGCCTGTGACCGTGTCCTGGAAC |
| | | | | TCTGGCGCTCTGACCAGCGGAGTGCACACC |
| | | | | TTCCCTGCTGTGCTGCAGTCCTCCGGCCTGT |
| | | | | ACTCCCTGTCCTCCGTCGTGACCGTGCCTTC |
| | | | | CAGCTCTCTGGGCACCCAGACCTACATCTG |
| | | | | CAACGTGAACCACAAGCCCTCCAACACCAA |
| | | | | GGTGGACAAGAAGGTGGAACCCAAGTCCT |
| | | | | GCGACAAGACCCACACCTGTCCCCCTTGTC |
| | | | | CTGCCCCTGAACTGCTGGGCGGACCTTCCG |
| | | | | TGTTCCTGTTCCCCCCAAAGCCCAAGGACA |
| | | | | CCCTGATGATCTCCCGGACCCCCGAAGTGA |
| | | | | CCTGCGTGGTGGTGGATGTGTCCCACGAGG |
| | | | | ACCCTGAAGTGAAGTTCAATTGGTACGTGG |
| | | | | ACGGCGTGGAAGTGCACAACGCCAAGACC |
| | | | | AAGCCTAGAGAGGAACAGTACAACTCCACC |
| | | | | TACCGGGTGGTGTCCGTGCTGACCGTGCTG |
| | | | | CACCAGGATTGGCTGAACGGCAAAGAGTAC |
| | | | | AAGTGCAAGGTGTCCAACAAGGCCCTGCCT |
| | | | | GCCCCCATCGAAAAGACCATCTCCAAGGCC |
| | | | | AAGGGCCAGCCCCGGGAACCCCAGGTGTAC |
| | | | | ACACTGCCCCCTAGCAGGGACGAGCTGACC |
| | | | | AAGAACCAGGTGTCCCTGACCTGTCTCGTG |
| | | | | AAAGGCTTCTACCCCTCCGATATCGCCGTG |
| | | | | GAATGGGAGTCCAACGGCCAGCCTGAGAA |
| | | | | CAACTACAAGACCACCCCCCCTGTGCTGGA |
| | | | | CTCCGACGGCTCATTCTTCCTGTACAGCAA |
| | | | | GCTGACAGTGGACAAGTCCCGGTGGCAGCA |
| | | | | GGGCAACGTGTTCTCCTGCTCCGTGATGCA |
| | | | | CGAGGCCCTGCACAACCACTACACCCAGAA |
| | | | | GTCCCTGTCCCTGAGCCCCGGCAAG |
| 142 | 413D08- | Amino acid | | QGIRND |
| | CDRL1 | sequence of CDRL1 | | |
| | (IMGT) | of 413D08 using | | |
| | | IMGT | | |
| 143 | 413D08- | Amino acid | | AAS |
| | CDRL2 | sequence of CDRL2 | | |
| | (IMGT) | of 413D08 using | | |
| | | IMGT | | |
| 144 | 413D08- | Amino acid | | LQHNSYPRT |
| | CDRL3 | sequence of CDRL3 | | |
| | (IMGT) | of 413D08 using | | |
| | | IMGT | | |
| 145 | 413D08- | Amino acid | | RASQGIRNDLG |
| | CDRL1 | sequence of CDRL1 | | |
| | (Kabat) | of 413D08 using | | |
| | | Kabat | | |
| 146 | 413D08- | Amino acid | | AASSLQS |
| | CDRL2 | sequence of CDRL2 | | |
| | (Kabat) | of 413D08 using | | |
| | | Kabat | | |
| 147 | 413D08- | Amino acid | | LQHNSYPRT |
| | CDRL3 | sequence of CDRL3 | | |
| | (Kabat) | of 413D08 using | | |
| | | Kabat | | |
| 148 | 413D08- | Amino acid | | DLQMTQSPSSLSASVGDRVTITCRASQGIRND |
| | Light | sequence of V_{L} of | | LGWYQQKPGKAPKRLIYAASSLQSGVPSRFS |
| | chain | 413D08 | | GSGSGTEFTLTISSLQPEDFATYYCLQHNSYPR |
| | variable | | | TFGQGTKVEIK |
| | region | | | |
| 149 | 413D08- | Nucleic acid | | GACCTCCAGATGACCCAGTCTCCATCCTCC |
| | Light | sequence of V_{L} of | | CTGTCTGCATCTGTAGGAGACAGAGTCACC |
| | chain | 413D08 | | ATCACTTGCCGGGCAAGTCAGGGCATTAGA |
| | variable | | | AATGATTTAGGCTGGTATCAGCAGAAACCA |
| | region | | | GGGAAAGCCCCTAAGCGCCTGATCTATGCT |
| | | | | GCATCCAGTTTGCAAAGTGGGGTCCCATCA |
| | | | | AGGTTCAGCGGCAGTGGATCTGGGACAGAA |
| | | | | TTCACTCTCACAATCAGCAGCCTGCAGCCT |
| | | | | GAAGATTTTGCAACTTATTACTGTCTACAGC |
| | | | | ATAATAGTTACCCTCGGACGTTCGGCCAAG |
| | | | | GGACCAAGGTGGAAATCAAAC |
| 150 | 413D08- | Amino acid | | DLQMTQSPSSLSASVGDRVTITCRASQGIRND |
| | full | sequence of 413D08 | | LGWYQQKPGKAPKRLIYAASSLQSGVPSRFS |
| | light | light chain | | GSGSGTEFTLTISSLQPEDFATYYCLQHNSYPR |
| | chain | | | TFGQGTKVEIKRTVAAPSVFIFPPSDEQLKSGT |
| | sequence | | | ASVVCLLNNFYPREAKVQWKVDNALQSGNS |
| | | | | QESVTEQDSKDSTYSLSSTLTLSKADYEKHKV |
| | | | | YACEVTHQGLSSPVTKSFNRGEC |
| 151 | 413D08- | Nucleic acid | | GACCTCCAGATGACCCAGTCTCCATCCTCC |
| | full | sequence of 413D08 | | CTGTCTGCATCTGTAGGAGACAGAGTCACC |
| | light | light chain | | ATCACTTGCCGGGCAAGTCAGGGCATTAGA |
| | chain | | | AATGATTTAGGCTGGTATCAGCAGAAACCA |
| | sequence | | | GGGAAAGCCCCTAAGCGCCTGATCTATGCT |
| | | | | GCATCCAGTTTGCAAAGTGGGGTCCCATCA |
| | | | | AGGTTCAGCGGCAGTGGATCTGGGACAGAA |
| | | | | TTCACTCTCACAATCAGCAGCCTGCAGCCT |
| | | | | GAAGATTTTGCAACTTATTACTGTCTACAGC |
| | | | | ATAATAGTTACCCTCGGACGTTCGGCCAAG |
| | | | | GGACCAAGGTGGAAATCAAACGTACGGTG |
| | | | | GCCGCTCCCTCCGTGTTCATCTTCCCACCTT |
| | | | | CCGACGAGCAGCTGAAGTCCGGCACCGCTT |
| | | | | CTGTCGTGTGCCTGCTGAACAACTTCTACCC |
| | | | | CCGCGAGGCCAAGGTGCAGTGGAAGGTGG |
| | | | | ACAACGCCCTGCAGTCCGGCAACTCCCAGG |
| | | | | AATCCGTGACCGAGCAGGACTCCAAGGACA |
| | | | | GCACCTACTCCCTGTCCTCCACCCTGACCCT |
| | | | | GTCCAAGGCCGACTACGAGAAGCACAAGG |
| | | | | TGTACGCCTGCGAAGTGACCCACCAGGGCC |
| | | | | TGTCTAGCCCCGTGACCAAGTCTTTCAACC |
| | | | | GGGGCGAGTGT |
| 152 | 386H03- | Amino acid | | GGSISSSDW |
| | CDRH1 | sequence of CDRH1 | | |
| | (IMGT) | of 386H03 using | | |
| | | IMGT | | |
| 153 | 386H03- | Amino acid | | IFHSGNT |
| | CDRH2 | sequence of CDRH2 | | |
| | (IMGT) | of 386H03 using | | |
| | | IMGT | | |
| 154 | 386H03- | Amino acid | | VRDGSGSY |
| | CDRH3 | sequence of CDRH3 | | |
| | (IMGT) | of 386H03 using | | |
| | | IMGT | | |
| 155 | 386H03- | Amino acid | | SSDWWS |
| | CDRH1 | sequence of CDRH1 | | |
| | (Kabat) | of 386H03 using | | |
| | | Kabat | | |
| 156 | 386H03- | Amino acid | | EIFHSGNTNYNPSLKS |
| | CDRH2 | sequence of CDRH2 | | |
| | (Kabat) | of 386H03 using | | |
| | | Kabat | | |
| 157 | 386H03- | Amino acid | | DGSGSY |
| | CDRH3 | sequence of CDRH3 | | |
| | (Kabat) | of 386H03 using | | |
| | | Kabat | | |
| 158 | 386H03- | Amino acid | | QVQLQESGPGLVKPSGTLSLTCAVSGGSISSS |
| | Heavy | sequence of V_{H} of | | DWWSWVRQPPGKGLEWIGEIFHSGNTNYNPS |
| | chain | 386H03 | | LKSRVTISVDKSKNQISLRLNSVTAADTAVYY |
| | variable | | | CVRDGSGSYWGQGTLVTVSS |
| | region | | | |
| 159 | 386H03- | Nucleic acid | | CAGGTGCAGCTGCAGGAGTCGGGCCCAGG |
| | Heavy | sequence of V_{H} of | | ACTGGTGAAGCCTTCGGGGACCCTGTCCCT |
| | chain | 386H03 | | CACCTGCGCTGTCTCTGGTGGCTCCATCAGC |
| | variable | | | AGTAGTGACTGGTGGAGTTGGGTCCGCCAG |
| | region | | | CCCCCAGGGAAGGGGCTGGAGTGGATTGG |
| | | | | GGAAATCTTTCATAGTGGGAACACCAACTA |
| | | | | CAACCCGTCCCTCAAGAGTCGAGTCACCAT |
| | | | | ATCAGTAGACAAGTCCAAGAACCAGATCTC |
| | | | | CCTGAGGCTGAACTCTGTGACCGCCGCGGA |
| | | | | CACGGCCGTGTATTACTGTGTGAGAGATGG |
| | | | | TTCGGGGAGTTACTGGGGCCAGGGAACCCT |
| | | | | GGTCACCGTCTCCTCAG |
| 160 | 386H03- | Amino acid | | QVQLQESGPGLVKPSGTLSLTCAVSGGSISSS |
| | full | sequence of 386H03 | | DWWSWVRQPPGKGLEWIGEIFHSGNTNYNPS |
| | heavy | heavy chain | | LKSRVTISVDKSKNQISLRLNSVTAADTAVYY |
| | chain | | | CVRDGSGSYWGQGTLVTVSSASTKGPSVFPL |
| | sequence | | | APSSKSTSGGTAALGCLVKDYFPEPVTVSWN |
| | | | | SGALTSGVHTFPAVLQSSGLYSLSSVVTVPSS |
| | | | | SLGTQTYICNVNHKPSNTKVDKKVEPKSCDK |
| | | | | THTCPPCPAPELLGGPSVFLFPPKPKDTLMISR |
| | | | | TPEVTCVVVDVSHEDPEVKFNWYVDGVEVH |
| | | | | NAKTKPREEQYNSTYRVVSVLTVLHQDWLN |
| | | | | GKEYKCKVSNKALPAPIEKTISKAKGQPREPQ |
| | | | | VYTLPPSRDELTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSL |
| | | | | SLSPGK |
| 161 | 386H03- | Nucleic acid | | CAGGTGCAGCTGCAGGAGTCGGGCCCAGG |
| | full | sequence of 386H03 | | ACTGGTGAAGCCTTCGGGGACCCTGTCCCT |
| | heavy | heavy chain | | CACCTGCGCTGTCTCTGGTGGCTCCATCAGC |
| | chain | | | AGTAGTGACTGGTGGAGTTGGGTCCGCCAG |
| | sequence | | | CCCCCAGGGAAGGGGCTGGAGTGGATTGG |
| | | | | GGAAATCTTTCATAGTGGGAACACCAACTA |
| | | | | CAACCCGTCCCTCAAGAGTCGAGTCACCAT |
| | | | | ATCAGTAGACAAGTCCAAGAACCAGATCTC |
| | | | | CCTGAGGCTGAACTCTGTGACCGCCGCGGA |
| | | | | CACGGCCGTGTATTACTGTGTGAGAGATGG |
| | | | | TTCGGGGAGTTACTGGGGCCAGGGAACCCT |
| | | | | GGTCACCGTCTCCTCAGCCAGCACCAAGGG |
| | | | | CCCCTCTGTGTTCCCTCTGGCCCCTTCCAGC |
| | | | | AAGTCCACCTCTGGCGGAACAGCCGCTCTG |
| | | | | GGCTGCCTCGTGAAGGACTACTTCCCCGAG |
| | | | | CCTGTGACCGTGTCCTGGAACTCTGGCGCT |
| | | | | CTGACCAGCGGAGTGCACACCTTCCCTGCT |
| | | | | GTGCTGCAGTCCTCCGGCCTGTACTCCCTGT |
| | | | | CCTCCGTCGTGACCGTGCCTTCCAGCTCTCT |
| | | | | GGGCACCCAGACCTACATCTGCAACGTGAA |
| | | | | CCACAAGCCCTCCAACACCAAGGTGGACAA |
| | | | | GAAGGTGGAACCCAAGTCCTGCGACAAGA |
| | | | | CCCACACCTGTCCCCCTTGTCCTGCCCCTGA |
| | | | | ACTGCTGGGCGGACCTTCCGTGTTCCTGTTC |
| | | | | CCCCCAAAGCCCAAGGACACCCTGATGATC |
| | | | | TCCCGGACCCCCGAAGTGACCTGCGTGGTG |
| | | | | GTGGATGTGTCCCACGAGGACCCTGAAGTG |
| | | | | AAGTTCAATTGGTACGTGGACGGCGTGGAA |
| | | | | GTGCACAACGCCAAGACCAAGCCTAGAGA |
| | | | | GGAACAGTACAACTCCACCTACCGGGTGGT |
| | | | | GTCCGTGCTGACCGTGCTGCACCAGGATTG |
| | | | | GCTGAACGGCAAAGAGTACAAGTGCAAGG |
| | | | | TGTCCAACAAGGCCCTGCCTGCCCCCATCG |
| | | | | AAAAGACCATCTCCAAGGCCAAGGGCCAG |
| | | | | CCCCGGGAACCCCAGGTGTACACACTGCCC |
| | | | | CCTAGCAGGGACGAGCTGACCAAGAACCA |
| | | | | GGTGTCCCTGACCTGTCTCGTGAAAGGCTT |
| | | | | CTACCCCTCCGATATCGCCGTGGAATGGGA |
| | | | | GTCCAACGGCCAGCCTGAGAACAACTACAA |
| | | | | GACCACCCCCCCTGTGCTGGACTCCGACGG |
| | | | | CTCATTCTTCCTGTACAGCAAGCTGACAGT |
| | | | | GGACAAGTCCCGGTGGCAGCAGGGCAACG |
| | | | | TGTTCTCCTGCTCCGTGATGCACGAGGCCCT |
| | | | | GCACAACCACTACACCCAGAAGTCCCTGTC |
| | | | | CCTGAGCCCCGGCAAG |
| 162 | 386H03- | Amino acid | | QSVLYSSNNKNY |
| | CDRL1 | sequence of CDRL1 | | |
| | (IMGT) | of 386H03 using | | |
| | | IMGT | | |
| 163 | 386H03- | Amino acid | | WAS |
| | CDRL2 | sequence of CDRL2 | | |
| | (IMGT) | of 386H03 using | | |
| | | IMGT | | |
| 164 | 386H03- | Amino acid | | QQYYSTRS |
| | CDRL3 | sequence of CDRL3 | | |
| | (IMGT) | of 386H03 using | | |
| | | IMGT | | |
| 165 | 386H03- | Amino acid | | KSSQSVLYSSNNKNYLA |
| | CDRL1 | sequence of CDRL1 | | |
| | (Kabat) | of 386H03 using | | |
| | | Kabat | | |
| 166 | 386H03- | Amino acid | | WASTRES |
| | CDRL2 | sequence of CDRL2 | | |
| | (Kabat) | of 386H03 using | | |
| | | Kabat | | |
| 167 | 386H03- | Amino acid | | QQYYSTRS |
| | CDRL3 | sequence of CDRL3 | | |
| | (Kabat) | of 386H03 using | | |
| | | Kabat | | |
| 168 | 386H03- | Amino acid | | DIVMTQSPDSLAVSLGERATINCKSSQSVLYS |
| | Light | sequence of V_{L} of | | SNNKNYLAWYQQKPGQPPKLLIYWASTRESG |
| | chain | 386H03 | | VPDRFSGSGSGTDFTLTISSLQAEDVAVYYCQ |
| | variable | | | QYYSTRSFGQGTKLEIK |
| | region | | | |
| 169 | 386H03- | Nucleic acid | | GACATCGTGATGACCCAGTCTCCAGACTCC |
| | Light | sequence of V_{L} of | | CTGGCTGTGTCTCTGGGCGAGAGGGCCACC |
| | chain | 386H03 | | ATCAACTGCAAGTCCAGCCAGAGTGTTTTA |
| | variable | | | TACAGCTCCAACAATAAGAACTACTTAGCT |
| | region | | | TGGTACCAGCAGAAACCAGGACAGCCTCCT |
| | | | | AAACTGCTCATTTACTGGGCATCTACCCGG |
| | | | | GAATCCGGGGTCCCTGACCGATTCAGTGGC |
| | | | | AGCGGGTCTGGGACAGATTTCACTCTCACC |
| | | | | ATCAGCAGCCTGCAGGCTGAAGATGTGGCA |
| | | | | gtttattactgtcagcaatattatagtactc |
| | | | | GCAGTTTTGGCCAGGGGACCAAGCTGGAGA |
| | | | | TCAAAC |
| 170 | 386H03- | Amino acid | | DIVMTQSPDSLAVSLGERATINCKSSQSVLYS |
| | full | sequence of 386H03 | | SNNKNYLAWYQQKPGQPPKLLIYWASTRESG |
| | light | light chain | | VPDRFSGSGSGTDFTLTISSLQAEDVAVYYCQ |
| | chain | | | QYYSTRSFGQGTKLEIKRTVAAPSVFIFPPSDE |
| | sequence | | | QLKSGTASVVCLLNNFYPREAKVQWKVDNA |
| | | | | LQSGNSQESVTEQDSKDSTYSLSSTLTLSKAD |
| | | | | YEKHKVYACEVTHQGLSSPVTKSFNRGEC |
| 171 | 386H03- | Nucleic acid | | GACATCGTGATGACCCAGTCTCCAGACTCC |
| | full | sequence of 386H03 | | CTGGCTGTGTCTCTGGGCGAGAGGGCCACC |
| | light | light chain | | ATCAACTGCAAGTCCAGCCAGAGTGTTTTA |
| | chain | | | TACAGCTCCAACAATAAGAACTACTTAGCT |
| | sequence | | | TGGTACCAGCAGAAACCAGGACAGCCTCCT |
| | | | | AAACTGCTCATTTACTGGGCATCTACCCGG |
| | | | | GAATCCGGGGTCCCTGACCGATTCAGTGGC |
| | | | | AGCGGGTCTGGGACAGATTTCACTCTCACC |
| | | | | ATCAGCAGCCTGCAGGCTGAAGATGTGGCA |
| | | | | gtttattactgtcagcaatattatagtactc |
| | | | | GCAGTTTTGGCCAGGGGACCAAGCTGGAGA |
| | | | | TCAAACGTACGGTGGCCGCTCCCTCCGTGT |
| | | | | TCATCTTCCCACCTTCCGACGAGCAGCTGA |
| | | | | AGTCCGGCACCGCTTCTGTCGTGTGCCTGCT |
| | | | | GAACAACTTCTACCCCCGCGAGGCCAAGGT |
| | | | | GCAGTGGAAGGTGGACAACGCCCTGCAGTC |
| | | | | CGGCAACTCCCAGGAATCCGTGACCGAGCA |
| | | | | GGACTCCAAGGACAGCACCTACTCCCTGTC |
| | | | | CTCCACCCTGACCCTGTCCAAGGCCGACTA |
| | | | | CGAGAAGCACAAGGTGTACGCCTGCGAAGT |
| | | | | GACCCACCAGGGCCTGTCTAGCCCCGTGAC |
| | | | | CAAGTCTTTCAACCGGGGCGAGTGT |
| 172 | 389A03- | Amino acid | | GGSISSSSYY |
| | CDRH1 | sequence of CDRH1 | | |
| | (IMGT) | of 389A03 using | | |
| | | IMGT | | |
| 173 | 389A03- | Amino acid | | IYSTGYT |
| | CDRH2 | sequence of CDRH2 | | |
| | (IMGT) | of 389A03 using | | |
| | | IMGT | | |
| 174 | 389A03- | Amino acid | | AISTAAGPEYFHR |
| | CDRH3 | sequence of CDRH3 | | |
| | (IMGT) | of 389A03 using | | |
| | | IMGT | | |
| 175 | 389A03- | Amino acid | | SSSYYCG |
| | CDRH1 | sequence of CDRH1 | | |
| | (Kabat) | of 389A03 using | | |
| | | Kabat | | |
| 176 | 389A03- | Amino acid | | SIYSTGYTYYNPSLKS |
| | CDRH2 | sequence of CDRH2 | | |
| | (Kabat) | of 389A03 using | | |
| | | Kabat | | |
| 177 | 389A03- | Amino acid | | STAAGPEYFHR |
| | CDRH3 | sequence of CDRH3 | | |
| | (Kabat) | of 389A03 using | | |
| | | Kabat | | |
| 178 | 389A03- | Amino acid | | QLQESGPGLVKPSETLSLTCTVSGGSISSSSYY |
| | Heavy | sequence of V_{H} of | | CGWIRQPPGKGLDWIGSIYSTGYTYYNPSLKS |
| | chain | 389A03 | | RVTISIDTSKNQFSCLILTSVTAADTAVYYCAI |
| | variable | | | STAAGPEYFHRWGQGTLVTVSS |
| | region | | | |
| 179 | 389A03- | Nucleic acid | | CAGCTGCAGGAGTCGGGCCCAGGCCTGGTG |
| | Heavy | sequence of V_{H} of | | AAGCCTTCGGAGACCCTGTCCCTCACCTGC |
| | chain | 389A03 | | ACTGTCTCTGGTGGCTCCATCAGCAGTAGT |
| | variable | | | AGTTATTACTGCGGCTGGATCCGCCAGCCC |
| | region | | | CCTGGGAAGGGGCTGGACTGGATTGGGAGT |
| | | | | ATCTATTCTACTGGGTACACCTACTACAACC |
| | | | | CGTCCCTCAAGAGTCGAGTCACCATTTCCA |
| | | | | TAGACACGTCCAAGAACCAGTTCTCATGCC |
| | | | | TGATACTGACCTCTGTGACCGCCGCAGACA |
| | | | | CGGCTGTGTATTACTGTGCGATAAGTACAG |
| | | | | CAGCTGGCCCTGAATACTTCCATCGCTGGG |
| | | | | GCCAGGGCACCCTGGTCACCGTCTCCTCAG |
| 180 | 389A03- | Amino acid | | QLQESGPGLVKPSETLSLTCTVSGGSISSSSYY |
| | full | sequence of 389A03 | | CGWIRQPPGKGLDWIGSIYSTGYTYYNPSLKS |
| | heavy | heavy chain | | RVTISIDTSKNQFSCLILTSVTAADTAVYYCAI |
| | chain | | | STAAGPEYFHRWGQGTLVTVSSASTKGPSVF |
| | sequence | | | PLAPSSKSTSGGTAALGCLVKDYFPEPVTVSW |
| | | | | NSGALTSGVHTFPAVLQSSGLYSLSSVVTVPS |
| | | | | SSLGTQTYICNVNHKPSNTKVDKKVEPKSCD |
| | | | | KTHTCPPCPAPELLGGPSVFLFPPKPKDTLMIS |
| | | | | RTPEVTCVVVDVSHEDPEVKFNWYVDGVEV |
| | | | | HNAKTKPREEQYNSTYRVVSVLTVLHQDWL |
| | | | | NGKEYKCKVSNKALPAPIEKTISKAKGQPREP |
| | | | | QVYTLPPSRDELTKNQVSLTCLVKGFYPSDIA |
| | | | | VEWESNGQPENNYKTTPPVLDSDGSFFLYSK |
| | | | | LTVDKSRWQQGNVFSCSVMHEALHNHYTQK |
| | | | | SLSLSPGK |
| 181 | 389A03- | Nucleic acid | | CAGCTGCAGGAGTCGGGCCCAGGCCTGGTG |
| | full | sequence of 389A03 | | AAGCCTTCGGAGACCCTGTCCCTCACCTGC |
| | heavy | heavy chain | | ACTGTCTCTGGTGGCTCCATCAGCAGTAGT |
| | chain | | | AGTTATTACTGCGGCTGGATCCGCCAGCCC |
| | sequence | | | CCTGGGAAGGGGCTGGACTGGATTGGGAGT |
| | | | | ATCTATTCTACTGGGTACACCTACTACAACC |
| | | | | CGTCCCTCAAGAGTCGAGTCACCATTTCCA |
| | | | | TAGACACGTCCAAGAACCAGTTCTCATGCC |
| | | | | TGATACTGACCTCTGTGACCGCCGCAGACA |
| | | | | CGGCTGTGTATTACTGTGCGATAAGTACAG |
| | | | | CAGCTGGCCCTGAATACTTCCATCGCTGGG |
| | | | | GCCAGGGCACCCTGGTCACCGTCTCCTCAG |
| | | | | CCAGCACCAAGGGCCCCTCTGTGTTCCCTCT |
| | | | | GGCCCCTTCCAGCAAGTCCACCTCTGGCGG |
| | | | | AACAGCCGCTCTGGGCTGCCTCGTGAAGGA |
| | | | | CTACTTCCCCGAGCCTGTGACCGTGTCCTGG |
| | | | | AACTCTGGCGCTCTGACCAGCGGAGTGCAC |
| | | | | ACCTTCCCTGCTGTGCTGCAGTCCTCCGGCC |
| | | | | TGTACTCCCTGTCCTCCGTCGTGACCGTGCC |
| | | | | TTCCAGCTCTCTGGGCACCCAGACCTACAT |
| | | | | CTGCAACGTGAACCACAAGCCCTCCAACAC |
| | | | | CAAGGTGGACAAGAAGGTGGAACCCAAGT |
| | | | | CCTGCGACAAGACCCACACCTGTCCCCCTT |
| | | | | GTCCTGCCCCTGAACTGCTGGGCGGACCTT |
| | | | | CCGTGTTCCTGTTCCCCCCAAAGCCCAAGG |
| | | | | ACACCCTGATGATCTCCCGGACCCCCGAAG |
| | | | | TGACCTGCGTGGTGGTGGATGTGTCCCACG |
| | | | | AGGACCCTGAAGTGAAGTTCAATTGGTACG |
| | | | | TGGACGGCGTGGAAGTGCACAACGCCAAG |
| | | | | ACCAAGCCTAGAGAGGAACAGTACAACTCC |
| | | | | ACCTACCGGGTGGTGTCCGTGCTGACCGTG |
| | | | | CTGCACCAGGATTGGCTGAACGGCAAAGAG |
| | | | | TACAAGTGCAAGGTGTCCAACAAGGCCCTG |
| | | | | CCTGCCCCCATCGAAAAGACCATCTCCAAG |
| | | | | GCCAAGGGCCAGCCCCGGGAACCCCAGGT |
| | | | | GTACACACTGCCCCCTAGCAGGGACGAGCT |
| | | | | GACCAAGAACCAGGTGTCCCTGACCTGTCT |
| | | | | CGTGAAAGGCTTCTACCCCTCCGATATCGC |
| | | | | CGTGGAATGGGAGTCCAACGGCCAGCCTGA |
| | | | | GAACAACTACAAGACCACCCCCCCTGTGCT |
| | | | | GGACTCCGACGGCTCATTCTTCCTGTACAG |
| | | | | CAAGCTGACAGTGGACAAGTCCCGGTGGCA |
| | | | | GCAGGGCAACGTGTTCTCCTGCTCCGTGAT |
| | | | | GCACGAGGCCCTGCACAACCACTACACCCA |
| | | | | GAAGTCCCTGTCCCTGAGCCCCGGCAAG |
| 182 | 389A03- | Amino acid | | QSVLYSSNSKNF |
| | CDRL1 | sequence of CDRL1 | | |
| | (IMGT) | of 389A03 using | | |
| | | IMGT | | |
| 183 | 389A03- | Amino acid | | WAS |
| | CDRL2 | sequence of CDRL2 | | |
| | (IMGT) | of 389A03 using | | |
| | | IMGT | | |
| 184 | 389A03- | Amino acid | | QQYYSTPRT |
| | CDRL3 | sequence of CDRL3 | | |
| | (IMGT) | of 389A03 using | | |
| | | IMGT | | |
| 185 | 389A03- | Amino acid | | KSSQSVLYSSNSKNFLA |
| | CDRL1 | sequence of CDRL1 | | |
| | (Kabat) | of 389A03 using | | |
| | | Kabat | | |
| 186 | 389A03- | Amino acid | | WASTRGS |
| | CDRL2 | sequence of CDRL2 | | |
| | (Kabat) | of 389A03 using | | |
| | | Kabat | | |
| 187 | 389A03- | Amino acid | | QQYYSTPRT |
| | CDRL3 | sequence of CDRL3 | | |
| | (Kabat) | of 389A03 using | | |
| | | Kabat | | |
| 188 | 389A03- | Amino acid | | DIVMTQSPDSLAVSLGERATINCKSSQSVLYS |
| | Light | sequence of V_{L} of | | SNSKNFLAWYQQKPGQPPKLFIYWASTRGSG |
| | chain | 389A03 | | VPDRISGSGSGTDFNLTISSLQAEDVAVYYCQ |
| | variable | | | QYYSTPRTFGQGTKVEIK |
| | region | | | |
| 189 | 389A03- | Nucleic acid | | GACATCGTGATGACCCAGTCTCCAGACTCC |
| | Light | sequence of V_{L} of | | CTGGCTGTGTCTCTGGGCGAGAGGGCCACC |
| | chain | 389A03 | | ATCAACTGCAAGTCCAGCCAGAGTGTTTTA |
| | variable | | | TACAGCTCCAACAGTAAGAACTTCTTAGCT |
| | region | | | TGGTACCAGCAGAAACCGGGACAGCCTCCT |
| | | | | AAGCTGTTCATTTACTGGGCATCTACCCGG |
| | | | | GGATCCGGGGTCCCTGACCGAATCAGTGGC |
| | | | | AGCGGGTCTGGGACAGATTTCAATCTCACC |
| | | | | ATCAGCAGCCTGCAGGCTGAAGATGTGGCA |
| | | | | GTTTATTACTGTCAACAATATTATAGTACTC |
| | | | | CTCGGACGTTCGGCCAAGGGACCAAGGTGG |
| | | | | AGATCAAAC |
| 190 | 389A03- | Amino acid | | DIVMTQSPDSLAVSLGERATINCKSSQSVLYS |
| | full | sequence of 389A03 | | SNSKNFLAWYQQKPGQPPKLFIYWASTRGSG |
| | light | light chain | | VPDRISGSGSGTDFNLTISSLQAEDVAVYYCQ |
| | chain | | | QYYSTPRTFGQGTKVEIKRTVAAPSVFIFPPSD |
| | sequence | | | EQLKSGTASVVCLLNNFYPREAKVQWKVDN |
| | | | | ALQSGNSQESVTEQDSKDSTYSLSSTLTLSKA |
| | | | | DYEKHKVYACEVTHQGLSSPVTKSFNRGEC |
| 191 | 389A03- | Nucleic acid | | GACATCGTGATGACCCAGTCTCCAGACTCC |
| | full | sequence of 389A03 | | CTGGCTGTGTCTCTGGGCGAGAGGGCCACC |
| | light | light chain | | ATCAACTGCAAGTCCAGCCAGAGTGTTTTA |
| | chain | | | TACAGCTCCAACAGTAAGAACTTCTTAGCT |
| | sequence | | | TGGTACCAGCAGAAACCGGGACAGCCTCCT |
| | | | | AAGCTGTTCATTTACTGGGCATCTACCCGG |
| | | | | GGATCCGGGGTCCCTGACCGAATCAGTGGC |
| | | | | AGCGGGTCTGGGACAGATTTCAATCTCACC |
| | | | | ATCAGCAGCCTGCAGGCTGAAGATGTGGCA |
| | | | | GTTTATTACTGTCAACAATATTATAGTACTC |
| | | | | CTCGGACGTTCGGCCAAGGGACCAAGGTGG |
| | | | | AGATCAAACGTACGGTGGCCGCTCCCTCCG |
| | | | | TGTTCATCTTCCCACCTTCCGACGAGCAGCT |
| | | | | GAAGTCCGGCACCGCTTCTGTCGTGTGCCT |
| | | | | GCTGAACAACTTCTACCCCCGCGAGGCCAA |
| | | | | GGTGCAGTGGAAGGTGGACAACGCCCTGCA |
| | | | | GTCCGGCAACTCCCAGGAATCCGTGACCGA |
| | | | | GCAGGACTCCAAGGACAGCACCTACTCCCT |
| | | | | GTCCTCCACCCTGACCCTGTCCAAGGCCGA |
| | | | | CTACGAGAAGCACAAGGTGTACGCCTGCGA |
| | | | | AGTGACCCACCAGGGCCTGTCTAGCCCCGT |
| | | | | GACCAAGTCTTTCAACCGGGGCGAGTGT |
| 192 | Human | IGHG | Heavy | |
| | IgG4 | *01 & | Chain | agcacctccgagagcacagccgccctgggctgcctggtcaaggacta |
| | heavy | IGHG | Constant | cttccccgaaccggtgacggtgtcgtggaactcaggcgccctgaccag |
| | chain | 4*04 | Region | cggcgtgcacaccttcccggctgtcctacagtcctcaggactctactcc |
| | constant | | Nucleotide | ctcagcagcgtggtgaccgtgccctccagcagcttgggcacgaagac |
| | region #1 | | Sequence | ctacacctgcaacgtagatcacaagcccagcaacaccaaggtggaca |
| | | | | agagagttgagtccaaatatggtcccccatgcccatcatgcccagcacc |
| | | | | tgagttcctggggggaccatcagtcttcctgttccccccaaaacccaag |
| | | | | gacactctcatgatctcccggacccctgaggtcacgtgcgtggtggtgg |
| | | | | acgtgagccaggaagaccccgaggtccagttcaactggtacgtggat |
| | | | | ggcgtggaggtgcataatgccaagacaaagccgcgggaggagcagt |
| | | | | tcaacagcacgtaccgtgtggtcagcgtcctcaccgtcctgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 193 | IgG4 | | Heavy | ASTKGPSVFPLAPCSRSTSESTAALGCLVKDY |
| | | | Chain | FPEPVTVSWNSGALTSGVHTFPAVLQSSGLYS |
| | | | Constant | LSSVVTVPSSSLGTKTYTCNVDHKPSNTKVD |
| | | | Region | KRVESKYGPPCPSCPAPEFLGGPSVFLFPPKPK |
| | | | Amino Acid | DTLMISRTPEVTCVVVDVSQEDPEVQFNWYV |
| | | | Sequence | DGVEVHNAKTKPREEQFNSTYRVVSVLTVLH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSRLTVDKSRWQEGNVFSCSVMHEALHN |
| | | | | HYTQKSLSLSLGK |
| 194 | Human | IGHG | Heavy | gcttccaccaagggcccatccgtcttccccctggcgccctgctccagg |
| | IgG4 | *02 | Chain | agcacctccgagagcacagccgccctgggctgcctggtcaaggacta |
| | heavy | | Constant | cttccccgaaccggtgacggtgtcgtggaactcaggcgccctgaccag |
| | chain | | Region | cggcgtgcacaccttcccggctgtcctacagtcctcaggactctactcc |
| | constant | | Nucleotide | ctcagcagcgtggtgaccgtgccctccagcagcttgggcacgaagac |
| | region #2 | | Sequence | ctacacctgcaacgtagatcacaagcccagcaacaccaaggtggaca |
| | | | | agagagttgagtccaaatatggtcccccgtgcccatcatgcccagcac |
| | | | | ctgagttcctggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcgtgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaggctaaccgtggacaagagcaggtggcaggaggggaatgtc |
| | | | | ttctcatgctccgtgatgcatgaggctctgcacaaccactacacgcaga |
| | | | | agagcctctccctgtctctgggtaaa |
| 195 | | | Heavy | ASTKGPSVFPLAPCSRSTSESTAALGCLVKDY |
| | | | Chain | FPEPVTVSWNSGALTSGVHTFPAVLQSSGLYS |
| | | | Constant | LSSVVTVPSSSLGTKTYTCNVDHKPSNTKVD |
| | | | Region | KRVESKYGPPCPSCPAPEFLGGPSVFLFPPKPK |
| | | | Amino Acid | DTLMISRTPEVTCVVVDVSQEDPEVQFNWYV |
| | | | Sequence | DGVEVHNAKTKPREEQFNSTYRVVSVLTVVH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSRLTVDKSRWQEGNVFSCSVMHEALHN |
| | | | | HYTQKSLSLSLGK |
| 196 | Human | IGHG | Heavy | gcttccaccaagggcccatccgtcttccccctggcgccctgctccagg |
| | IgG4 | *03 | Chain | agcacctccgagagcacagccgccctgggctgcctggtcaaggacta |
| | heavy | | Constant | cttccccgaaccggtgacggtgtcgtggaactcaggcgccctgaccag |
| | chain | | Region | cggcgtgcacaccttcccggctgtcctacagtcctcaggactctactcc |
| | constant | | Nucleotide | ctcagcagcgtggtgaccgtgccctccagcagcttgggcacgaagac |
| | region #3 | | Sequence | ctacacctgcaacgtagatcacaagcccagcaacaccaaggtggaca |
| | | | | agagagttgagtccaaatatggtcccccatgcccatcatgcccagcacc |
| | | | | tgagttcctggggggaccatcagtcttcctgttccccccaaaacccaag |
| | | | | gacactctcatgatctcccggacccctgaggtcacgtgcgtggtggtgg |
| | | | | acgtgagccaggaagaccccgaggtccagttcaactggtacgtggat |
| | | | | ggcgtggaggtgcataatgccaagacaaagccgcgggaggagcagt |
| | | | | tcaacagcacgtaccgtgtggtcagcgtcctcaccgtcctgcaccagg |
| | | | | actggctgaacggcaaggagtacaagtgcaaggtctccaacaaaggc |
| | | | | ctcccgtcctccatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggctccttcttcctcta |
| | | | | cagcaagctcaccgtggacaagagcaggtggcaggaggggaacgtc |
| | | | | ttctcatgctccgtgatgcatgaggctctgcacaaccactacacgcaga |
| | | | | agagcctctccctgtctctgggtaaa |
| 197 | IgG4 | | Heavy | ASTKGPSVFPLAPCSRSTSESTAALGCLVKDY |
| | | | Chain | FPEPVTVSWNSGALTSGVHTFPAVLQSSGLYS |
| | | | Constant | LSSVVTVPSSSLGTKTYTCNVDHKPSNTKVD |
| | | | Region | KRVESKYGPPCPSCPAPEFLGGPSVFLFPPKPK |
| | | | Amino Acid | DTLMISRTPEVTCVVVDVSQEDPEVQFNWYV |
| | | | Sequence | DGVEVHNAKTKPREEQFNSTYRVVSVLTVLH |
| | | | | QDWLNGKEYKCKVSNKGLPSSIEKTISKAKG |
| | | | | QPREPQVYTLPPSQEEMTKNQVSLTCLVKGF |
| | | | | YPSDIAVEWESNGQPENNYKTTPPVLDSDGSF |
| | | | | FLYSKLTVDKSRWQEGNVFSCSVMHEALHN |
| | | | | HYTQKSLSLSLGK |
| 198 | IgG4 | - | Heavy | gcctccaccaagggcccatccgtcttccccctggcgccctgctccagg |
| | heavy | IgG4- | Chain | agcacctccgagagcacggccgccctgggctgcctggtcaaggacta |
| | chain | PE | Constant | cttccccgaaccagtgacggtgtcgtggaactcaggcgccctgaccag |
| | constant | | Region | cggcgtgcacaccttcccggctgtcctacagtcctcaggactctactcc |
| | region- | | Nucleotide | ctcagcagcgtggtgaccgtgccctccagcagcttgggcacgaagac |
| | IgG4-PE | | Sequence- | ctacacctgcaacgtagatcacaagcccagcaacaccaaggtggaca |
| | | | Synthetic | agagagttgagtccaaatatggtcccccatgcccaccatgcccagcgc |
| | | | Version A | ctgaatttgaggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcctgcaccagg |
| | | | | actggctgaacggcaaggagtacaagtgcaaggtctccaacaaaggc |
| | | | | ctcccgtcatcgatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggatccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 199 | IgG4 | | Heavy | ASTKGPSVFPLAPCSRSTSESTAALGCLVKDY |
| | heavy | | Chain | FPEPVTVSWNSGALTSGVHTFPAVLQSSGLYS |
| | chain | | Constant | LSSVVTVPSSSLGTKTYTCNVDHKPSNTKVD |
| | constant | | Region | KRVESKYGPPCPPCPAPEFEGGPSVFLFPPKPK |
| | region- | | Amino Acid | DTLMISRTPEVTCVVVDVSQEDPEVQFNWYV |
| | IgG4-PE | | Sequence- | DGVEVHNAKTKPREEQFNSTYRVVSVLTVLH |
| | | | Encoded by | QDWLNGKEYKCKVSNKGLPSSIEKTISKAKG |
| | | | Synthetic | QPREPQVYTLPPSQEEMTKNQVSLTCLVKGF |
| | | | Version A, | YPSDIAVEWESNGQPENNYKTTPPVLDSDGSF |
| | | | B & C(Two | FLYSRLTVDKSRWQEGNVFSCSVMHEALHN |
| | | | residues | HYTQKSLSLSLGK |
| | | | that differ | |
| | | | from the | |
| | | | wild-type | |
| | | | sequence | |
| | | | are | |
| | | | identified in | |
| | | | bold) | |
| 200 | IgG4 | | Heavy | Gcctccaccaagggacctagcgtgttccctctcgccccctgttccaggt |
| | heavy | | Chain | ccacaagcgagtccaccgctgccctcggctgtctggtgaaagactactt |
| | chain | | Constant | tcccgagcccgtgaccgtctcctggaatagcggagccctgacctccgg |
| | constant | | Region | cgtgcacacatttcccgccgtgctgcagagcagcggactgtatagcct |
| | region- | | Nucleotide | gagcagcgtggtgaccgtgcccagctccagcctcggcaccaaaacct |
| | IgG4-PE | | Sequence- | acacctgcaacgtggaccacaagccctccaacaccaaggtggacaag |
| | | | Synthetic | cgggtggagagcaagtacggccccccttgccctccttgtcctgcccct |
| | | | Version B | gagttcgagggaggaccctccgtgttcctgtttccccccaaacccaagg |
| | | | | acaccctgatgatctcccggacacccgaggtgacctgtgtggtcgtgg |
| | | | | acgtcagccaggaggaccccgaggtgcagttcaactggtatgtggac |
| | | | | ggcgtggaggtgcacaatgccaaaaccaagcccagggaggagcagt |
| | | | | tcaattccacctacagggtggtgagcgtgctgaccgtcctgcatcagga |
| | | | | ttggctgaacggcaaggagtacaagtgcaaggtgtccaacaagggac |
| | | | | tgcccagctccatcgagaagaccatcagcaaggctaagggccagccg |
| | | | | agggagccccaggtgtataccctgcctcctagccaggaagagatgac |
| | | | | caagaaccaagtgtccctgacctgcctggtgaagggattctacccctcc |
| | | | | gacatcgccgtggagtgggagagcaatggccagcccgagaacaact |
| | | | | acaaaacaacccctcccgtgctcgatagcgacggcagcttctttctctac |
| | | | | agccggctgacagtggacaagagcaggtggcaggagggcaacgtgt |
| | | | | tctcctgttccgtgatgcacgaggccctgcacaatcactacacccagaa |
| | | | | gagcctctccctgtccctgggcaag |
| 201 | IgG4 | | Heavy | gccagcaccaagggcccttccgtgttccccctggccccttgcagcagg |
| | heavy | | Chain | agcacctccgaatccacagctgccctgggctgtctggtgaaggactact |
| | chain | | Constant | ttcccgagcccgtgaccgtgagctggaacagcggcgctctgacatccg |
| | constant | | Region | gcgtccacacctttcctgccgtcctgcagtcctccggcctctactccctgt |
| | region- | | Nucleotide | cctccgtggtgaccgtgcctagctcctccctcggcaccaagacctacac |
| | IgG4-PE | | Sequence- | ctgtaacgtggaccacaaaccctccaacaccaaggtggacaaacggg |
| | | | Synthetic | tcgagagcaagtacggccctccctgccctccttgtcctgcccccgagtt |
| | | | Version C | cgaaggcggacccagcgtgttcctgttccctcctaagcccaaggacac |
| | | | | cctcatgatcagccggacacccgaggtgacctgcgtggtggtggatgt |
| | | | | gagccaggaggaccctgaggtccagttcaactggtatgtggatggcgt |
| | | | | ggaggtgcacaacgccaagacaaagccccgggaagagcagttcaac |
| | | | | tccacctacagggtggtcagcgtgctgaccgtgctgcatcaggactgg |
| | | | | ctgaacggcaaggagtacaagtgcaaggtcagcaataagggactgcc |
| | | | | cagcagcatcgagaagaccatctccaaggctaaaggccagccccgg |
| | | | | gaacctcaggtgtacaccctgcctcccagccaggaggagatgaccaa |
| | | | | gaaccaggtgagcctgacctgcctggtgaagggattctacccttccga |
| | | | | catcgccgtggagtgggagtccaacggccagcccgagaacaattata |
| | | | | agaccacccctcccgtcctcgacagcgacggatccttctttctgtactcc |
| | | | | aggctgaccgtggataagtccaggtggcaggaaggcaacgtgttcag |
| | | | | ctgctccgtgatgcacgaggccctgcacaatcactacacccagaagtc |
| | | | | cctgagcctgtccctgggaaag |
| 202 | IgG4 | | Heavy | gcctccaccaagggcccatccgtcttccccctggcgccctgctccagg |
| | heavy | | Chain | agcacctccgagagcacggccgccctgggctgcctggtcaaggacta |
| | chain | | Constant | cttccccgaaccagtgacggtgtcgtggaactcaggcgccctgaccag |
| | constant | | Region | cggcgtgcacaccttcccggctgtcctacagtcctcaggactctactcc |
| | region | | Nucleotide | ctcagcagcgtggtgaccgtgccctccagcagcttgggcacgaagac |
| | | | Sequence- | ctacacctgcaacgtagatcacaagcccagcaacaccaaggtggaca |
| | | | Synthetic | agagagttgagtccaaatatggtcccccatgcccaccatgcccagcgc |
| | | | Version D | ctccagttgcggggggaccatcagtcttcctgttccccccaaaacccaa |
| | | | | ggacactctcatgatctcccggacccctgaggtcacgtgcgtggtggtg |
| | | | | gacgtgagccaggaagaccccgaggtccagttcaactggtacgtgga |
| | | | | tggcgtggaggtgcataatgccaagacaaagccgcgggaggagcag |
| | | | | ttcaacagcacgtaccgtgtggtcagcgtcctcaccgtcctgcaccagg |
| | | | | actggctgaacggcaaggagtacaagtgcaaggtctccaacaaaggc |
| | | | | ctcccgtcatcgatcgagaaaaccatctccaaagccaaagggcagccc |
| | | | | cgagagccacaggtgtacaccctgcccccatcccaggaggagatgac |
| | | | | caagaaccaggtcagcctgacctgcctggtcaaaggcttctaccccag |
| | | | | cgacatcgccgtggagtgggagagcaatgggcagccggagaacaac |
| | | | | tacaagaccacgcctcccgtgctggactccgacggatccttcttcctcta |
| | | | | cagcaggctaaccgtggacaagagcaggtggcaggaggggaatgtc |
| | | | | ttctcatgctccgtgatgcatgaggctctgcacaaccactacacacaga |
| | | | | agagcctctccctgtctctgggtaaa |
| 203 | | | Heavy | ASTKGPSVFPLAPCSRSTSESTAALGCLVKDY |
| | | | Chain | FPEPVTVSWNSGALTSGVHTFPAVLQSSGLYS |
| | | | Constant | LSSVVTVPSSSLGTKTYTCNVDHKPSNTKVD |
| | | | Region | KRVESKYGPPCPPCPAPPVAGGPSVFLFPPKP |
| | | | Amino Acid | KDTLMISRTPEVTCVVVDVSQEDPEVQFNWY |
| | | | Sequence- | VDGVEVHNAKTKPREEQFNSTYRVVSVLTVL |
| | | | Encoded by | HQDWLNGKEYKCKVSNKGLPSSIEKTISKAK |
| | | | Synthetic | GQPREPQVYTLPPSQEEMTKNQVSLTCLVKG |
| | | | Version D | FYPSDIAVEWESNGQPENNYKTTPPVLDSDGS |
| | | | | FFLYSRLTVDKSRWQEGNVFSCSVMHEALHN |
| | | | | HYTQKSLSLSLGK |
| 204 | Disabled | Dis- | Heavy | gcctccaccaagggcccatcggtcttccccctggcaccctcctccaag |
| | Human | abled | Chain | agcacctctgggggcacagcggccctgggctgcctggtcaaggacta |
| | IgG1 | IGHG | Constant | cttccccgaaccggtgacggtgtcgtggaactcaggcgccctgaccag |
| | heavy | 1 | Region | cggcgtgcacaccttcccggctgtcctacagtcctcaggactctactcc |
| | chain | | Nucleotide | ctcagcagcgtggtgaccgtgccctccagcagcttgggcacccagac |
| | constant | | Sequence | ctacatctgcaacgtgaatcacaagcccagcaacaccaaggtggacaa |
| | region | | | gaaagtggagcccaaatcttgtgacaaaactcacacatgcccaccgtg |
| | | | | cccagcacctgaactcgcgggggcaccgtcagtcttcctcttcccccca |
| | | | | aaacccaaggacaccctcatgatctcccggacccctgaggtcacatgc |
| | | | | gtggtggtggacgtgagccacgaagaccctgaggtcaagttcaactgg |
| | | | | tacgtggacggcgtggaggtgcataatgccaagacaaagccgcggg |
| | | | | aggagcagtacaacagcacgtaccgtgtggtcagcgtcctcaccgtcc |
| | | | | tgcaccaggactggctgaatggcaaggagtacaagtgcaaggtctcca |
| | | | | acaaagccctcccagcccccatcgagaaaaccatctccaaagccaaa |
| | | | | gggcagccccgagaaccacaggtgtacaccctgcccccatcccggg |
| | | | | atgagctgaccaagaaccaggtcagcctgacctgcctggtcaaaggct |
| | | | | tctatcccagcgacatcgccgtggagtgggagagcaatgggcagccg |
| | | | | gagaacaactacaagaccacgcctcccgtgctggactccgacggctc |
| | | | | cttcttcctctacagcaagctcaccgtggacaagagcaggtggcagca |
| | | | | ggggaacgtcttctcatgctccgtgatgcatgaggctctgcacaaccac |
| | | | | tacacgcagaagagcctctccctgtctccgggtaaa |
| 205 | | | Heavy | ASTKGPSVFPLAPSSKSTSGGTAALGCLVKDY |
| | | | Chain | FPEPVTVSWNSGALTSGVHTFPAVLQSSGLYS |
| | | | Constant | LSSVVTVPSSSLGTQTYICNVNHKPSNTKVDK |
| | | | Region | KVEPKSCDKTHTCPPCPAPELAGAPSVFLFPP |
| | | | Amino Acid | KPKDTLMISRTPEVTCVVVDVSHEDPEVKFN |
| | | | Sequence | WYVDGVEVHNAKTKPREEQYNSTYRVVSVL |
| | | | (Two | TVLHQDWLNGKEYKCKVSNKALPAPIEKTIS |
| | | | residues | KAKGQPREPQVYTLPPSRDELTKNQVSLTCL |
| | | | that differ | VKGFYPSDIAVEWESNGQPENNYKTTPPVLD |
| | | | from the | SDGSFFLYSKLTVDKSRWQQGNVFSCSVMHE |
| | | | wild-type | ALHNHYTQKSLSLSPGK |
| | | | sequence | |
| | | | are | |
| | | | identified in | |
| | | | bold) | |
| 206 | Human | IGKC | Cκ Light | cgtacggtggccgctccctccgtgttcatcttcccaccttccgacgagca |
| | Cκ | *01 | Chain | gctgaagtccggcaccgcttctgtcgtgtgcctgctgaacaacttctacc |
| | constant | | Constant | cccgcgaggccaaggtgcagtggaaggtggacaacgccctgcagtc |
| | region | | Region | cggcaactcccaggaatccgtgaccgagcaggactccaaggacagc |
| | | | Nucleotide | acctactccctgtcctccaccctgaccctgtccaaggccgactacgaga |
| | | | Sequence | agcacaaggtgtacgcctgcgaagtgacccaccagggcctgtctagc |
| | | | | cccgtgaccaagtctttcaaccggggcgagtgt |
| 207 | | | Cκ Light | RTVAAPSVFIFPPSDEQLKSGTASVVCLLNNF |
| | | | Chain | YPREAKVQWKVDNALQSGNSQESVTEQDSK |
| | | | Constant | DSTYSLSSTLTLSKADYEKHKVYACEVTHQG |
| | | | Region | LSSPVTKSFNRGEC |
| | | | Amino Acid | |
| | | | Sequence | |
| 208 | Human Cκ | IGKC | Cκ Light | cgaactgtggctgcaccatctgtcttcatcttcccgccatctgatgagca |
| | constant | *02 | Chain | gttgaaatctggaactgcctctgttgtgtgcctgctgaataacttctatccc |
| | region | | Constant | agagaggccaaagtacagtggaaggtggataacgccctccaatcggg |
| | | | Region | taactcccaggagagtgtcacagagcaggagagcaaggacagcacct |
| | | | Nucleotide | acagcctcagcagcaccctgacgctgagcaaagcagactacgagaaa |
| | | | Sequence | cacaaagtctacgccggcgaagtcacccatcagggcctgagctcgcc |
| | | | | cgtcacaaagagcttcaacaggggagagtgt |
| 209 | | | Cκ Light | RTVAAPSVFIFPPSDEQLKSGTASVVCLLNNF |
| | | | Chain | YPREAKVQWKVDNALQSGNSQESVTEQESK |
| | | | Constant | DSTYSLSSTLTLSKADYEKHKVYAGEVTHQG |
| | | | Region | LSSPVTKSFNRGEC |
| | | | Amino Acid | |
| | | | Sequence | |
| 210 | Human Cκ | IGKC | Cκ Light | cgaactgtggctgcaccatctgtcttcatcttcccgccatctgatgagca |
| | constant | *03 | Chain | gttgaaatctggaactgcctctgttgtgtgcctgctgaataacttctatccc |
| | region | | Constant | agagaggccaaagtacagcggaaggtggataacgccctccaatcgg |
| | | | Region | gtaactcccaggagagtgtcacagagcaggagagcaaggacagcac |
| | | | Nucleotide | ctacagcctcagcagcaccctgacgctgagcaaagcagactacgaga |
| | | | Sequence | aacacaaagtctacgcctgcgaagtcacccatcagggcctgagctcgc |
| | | | | ccgtcacaaagagcttcaacaggggagagtgt |
| 211 | | | Cκ Light | RTVAAPSVFIFPPSDEQLKSGTASVVCLLNNF |
| | | | Chain | YPREAKVQRKVDNALQSGNSQESVTEQESKD |
| | | | Constant | STYSLSSTLTLSKADYEKHKVYACEVTHQGL |
| | | | Region | SSPVTKSFNRGEC |
| | | | Amino Acid | |
| | | | Sequence | |
| 212 | Human Cκ | IGKC | Cκ Light | cgaactgtggctgcaccatctgtcttcatcttcccgccatctgatgagca |
| | constant | *04 | Chain | gttgaaatctggaactgcctctgttgtgtgcctgctgaataacttctatccc |
| | region | | Constant | agagaggccaaagtacagtggaaggtggataacgccctccaatcggg |
| | | | Region | taactcccaggagagtgtcacagagcaggacagcaaggacagcacct |
| | | | Nucleotide | acagcctcagcagcaccctgacgctgagcaaagcagactacgagaaa |
| | | | Sequence | cacaaactctacgcctgcgaagtcacccatcagggcctgagctcgccc |
| | | | | gtcacaaagagcttcaacaggggagagtgt |
| 213 | | | Cκ Light | RTVAAPSVFIFPPSDEQLKSGTASVVCLLNNF |
| | | | Chain | YPREAKVQWKVDNALQSGNSQESVTEQDSK |
| | | | Constant | DSTYSLSSTLTLSKADYEKHKLYACEVTHQG |
| | | | Region | LSSPVTKSFNRGEC |
| | | | Amino Acid | |
| | | | Sequence | |
| 214 | Human Cκ | IGKC | Cκ Light | cgaactgtggctgcaccatctgtcttcatcttcccgccatctgatgagca |
| | constant | *05 | Chain | gttgaaatctggaactgcctctgttgtgtgcctgctgaataacttctatccc |
| | region | | Constant | agagaggccaaagtacagtggaaggtggataacgccctccaatcggg |
| | | | Region | taactcccaggagagtgtcacagagcaggacagcaaggacagcacct |
| | | | Nucleotide | acagcctcagcaacaccctgacgctgagcaaagcagactacgagaaa |
| | | | Sequence | cacaaagtctacgcctgcgaagtcacccatcagggcctgagctcgccc |
| | | | | gtcacaaagagcttcaacaggggagagtgc |
| 215 | | | Cκ Light | RTVAAPSVFIFPPSDEQLKSGTASVVCLLNNF |
| | | | Chain | YPREAKVQWKVDNALQSGNSQESVTEQDSK |
| | | | Constant | DSTYSLSNTLTLSKADYEKHKVYACEVTHQG |
| | | | Region | LSSPVTKSFNRGEC |
| | | | Amino Acid | |
| | | | Sequence | |
| 216 | Human Cλ | IGCλ | Cλ Light | cccaaggccaaccccacggtcactctgttcccgccctcctctgaggag |
| | constant | 1*01 | Chain | ctccaagccaacaaggccacactagtgtgtctgatcagtgacttctacc |
| | region | | Constant | cgggagctgtgacagtggcttggaaggcagatggcagccccgtcaag |
| | | | Region | gcgggagtggagacgaccaaaccctccaaacagagcaacaacaagt |
| | | | Nucleotide | acgcggccagcagctacctgagcctgacgcccgagcagtggaagtc |
| | | | Sequence | ccacagaagctacagctgccaggtcacgcatgaagggagcaccgtg |
| | | | | gagaagacagtggcccctacagaatgttca |
| 217 | | | Cλ Light | PKANPTVTLFPPSSEELQANKATLVCLISDFYP |
| | | | Chain | GAVTVAWKADGSPVKAGVETTKPSKQSNNK |
| | | | Constant | YAASSYLSLTPEQWKSHRSYSCQVTHEGSTV |
| | | | Region | EKTVAPTECS |
| | | | Amino Acid | |
| | | | Sequence | |
| 218 | Human Cλ | IGCλ | Cλ Light | ggtcagcccaaggccaaccccactgtcactctgttcccgccctcctctg |
| | constant | 1*02 | Chain | aggagctccaagccaacaaggccacactagtgtgtctgatcagtgactt |
| | region | | Constant | ctacccgggagctgtgacagtggcctggaaggcagatggcagcccc |
| | | | Region | gtcaaggcgggagtggagaccaccaaaccctccaaacagagcaaca |
| | | | Nucleotide | acaagtacgcggccagcagctacctgagcctgacgcccgagcagtg |
| | | | Sequence | gaagtcccacagaagctacagctgccaggtcacgcatgaagggagca |
| | | | | ccgtggagaagacagtggcccctacagaatgttca |
| 219 | | | Cλ Light | GQPKANPTVTLFPPSSEELQANKATLVCLISD |
| | | | Chain | FYPGAVTVAWKADGSPVKAGVETTKPSKQS |
| | | | Constant | NNKYAASSYLSLTPEQWKSHRSYSCQVTHEG |
| | | | Region | STVEKTVAPTECS |
| | | | Amino Acid | |
| | | | Sequence | |
| 220 | Human Cλ | IGCλ | Cλ Light | ggtcagcccaaggccaaccccactgtcactctgttcccgccctcctctg |
| | constant | 2*01 | Chain | aggagctccaagccaacaaggccacactagtgtgtctgatcagtgactt |
| | region | | Constant | ctacccgggagctgtgacagtggcctggaaggcagatggcagcccc |
| | | | Region | gtcaaggcgggagtggagaccaccaaaccctccaaacagagcaaca |
| | | | Nucleotide | acaagtacgcggccagcagctacctgagcctgacgcccgagcagtg |
| | | | Sequence- | gaagtcccacagaagctacagctgccaggtcacgcatgaagggagca |
| | | | Version A | ccgtggagaagacagtggcccctacagaatgttca |
| 221 | | | Cλ Light | ggccagcctaaggccgctccttctgtgaccctgttccccccatcctccg |
| | | | Chain | aggaactgcaggctaacaaggccaccctcgtgtgcctgatcagcgact |
| | | | Constant | tctaccctggcgccgtgaccgtggcctggaaggctgatagctctcctgt |
| | | | Region | gaaggccggcgtggaaaccaccaccccttccaagcagtccaacaaca |
| | | | Nucleotide | aatacgccgcctcctcctacctgtccctgacccctgagcagtggaagtc |
| | | | Sequence- | ccaccggtcctacagctgccaagtgacccacgagggctccaccgtgg |
| | | | Version B | aaaagaccgtggctcctaccgagtgctcc |
| 222 | | | Cλ Light | ggccagcctaaagctgcccccagcgtcaccctgtttcctccctccagcg |
| | | | Chain | aggagctccaggccaacaaggccaccctcgtgtgcctgatctccgact |
| | | | Constant | tctatcccggcgctgtgaccgtggcttggaaagccgactccagccctgt |
| | | | Region | caaagccggcgtggagaccaccacaccctccaagcagtccaacaac |
| | | | Nucleotide | aagtacgccgcctccagctatctctccctgacccctgagcagtggaagt |
| | | | Sequence- | cccaccggtcctactcctgtcaggtgacccacgagggctccaccgtgg |
| | | | Version C | aaaagaccgtcgcccccaccgagtgctcc |
| 223 | | | Cλ Light | GQPKANPTVTLFPPSSEELQANKATLVCLISD |
| | | | Chain | FYPGAVTVAWKADGSPVKAGVETTKPSKQS |
| | | | Constant | NNKYAASSYLSLTPEQWKSHRSYSCQVTHEG |
| | | | Region | STVEKTVAPTECS |
| | | | Amino Acid | |
| | | | Sequence- | |
| | | | Encoded by | |
| | | | Version A, | |
| | | | B&C | |
| 224 | Human Cλ | IGCλ | Cλ Light | ggtcagcccaaggctgccccctcggtcactctgttcccgccctcctctg |
| | constant | 2*02 & | Chain | aggagcttcaagccaacaaggccacactggtgtgtctcataagtgactt |
| | region | IGLC | Constant | ctacccgggagccgtgacagtggcctggaaggcagatagcagcccc |
| | | 2*03 | Region | gtcaaggcgggagtggagaccaccacaccctccaaacaaagcaaca |
| | | | Nucleotide | acaagtacgcggccagcagctatctgagcctgacgcctgagcagtgg |
| | | | Sequence | aagtcccacagaagctacagctgccaggtcacgcatgaagggagcac |
| | | | | cgtggagaagacagtggcccctacagaatgttca |
| 225 | | | Cλ Light | GQPKAAPSVTLFPPSSEELQANKATLVCLISDF |
| | | | Chain | YPGAVTVAWKADSSPVKAGVETTTPSKQSN |
| | | | Constant | NKYAASSYLSLTPEQWKSHRSYSCQVTHEGS |
| | | | Region | TVEKTVAPTECS |
| | | | Amino Acid | |
| | | | Sequence | |
| 226 | Human Cλ | IGCλ | Cλ Light | cccaaggctgccccctcggtcactctgttcccaccctcctctgaggagc |
| | constant | 3*01 | Chain | ttcaagccaacaaggccacactggtgtgtctcataagtgacttctacccg |
| | region | | Constant | ggagccgtgacagttgcctggaaggcagatagcagccccgtcaaggc |
| | | | Region | gggggtggagaccaccacaccctccaaacaaagcaacaacaagtac |
| | | | Nucleotide | gcggccagcagctacctgagcctgacgcctgagcagtggaagtccca |
| | | | Sequence | caaaagctacagctgccaggtcacgcatgaagggagcaccgtggag |
| | | | | aagacagttgcccctacggaatgttca |
| 227 | | | Cλ Light | PKAAPSVTLFPPSSEELQANKATLVCLISDFYP |
| | | | Chain | GAVTVAWKADSSPVKAGVETTTPSKQSNNK |
| | | | Constant | YAASSYLSLTPEQWKSHKSYSCQVTHEGSTV |
| | | | Region | EKTVAPTECS |
| | | | Amino Acid | |
| | | | Sequence | |
| 228 | Human Cλ | IGCλ | Cλ Light | ggtcagcccaaggctgccccctcggtcactctgttcccaccctcctctg |
| | constant | 3*02 | Chain | aggagcttcaagccaacaaggccacactggtgtgtctcataagtgactt |
| | region | | Constant | ctacccggggccagtgacagttgcctggaaggcagatagcagccccg |
| | | | Region | tcaaggcgggggtggagaccaccacaccctccaaacaaagcaacaa |
| | | | Nucleotide | caagtacgcggccagcagctacctgagcctgacgcctgagcagtgga |
| | | | Sequence | agtcccacaaaagctacagctgccaggtcacgcatgaagggagcacc |
| | | | | gtggagaagacagtggcccctacggaatgttca |
| 229 | | | Cλ Light | GQPKAAPSVTLFPPSSEELQANKATLVCLISDF |
| | | | Chain | YPGPVTVAWKADSSPVKAGVETTTPSKQSNN |
| | | | Constant | KYAASSYLSLTPEQWKSHKSYSCQVTHEGST |
| | | | Region | VEKTVAPTECS |
| | | | Amino Acid | |
| | | | Sequence | |
| 230 | Human Cλ | IGCλ | Cλ Light | ggtcagcccaaggctgccccctcggtcactctgttcccaccctcctctg |
| | constant | 3*03 | Chain | aggagcttcaagccaacaaggccacactggtgtgtctcataagtgactt |
| | region | | Constant | ctacccgggagccgtgacagtggcctggaaggcagatagcagcccc |
| | | | Region | gtcaaggcgggagtggagaccaccacaccctccaaacaaagcaaca |
| | | | Nucleotide | acaagtacgcggccagcagctacctgagcctgacgcctgagcagtgg |
| | | | Sequence | aagtcccacaaaagctacagctgccaggtcacgcatgaagggagcac |
| | | | | cgtggagaagacagtggcccctacagaatgttca |
| 231 | | | Cλ Light | GQPKAAPSVTLFPPSSEELQANKATLVCLISDF |
| | | | Chain | YPGAVTVAWKADSSPVKAGVETTTPSKQSN |
| | | | Constant | NKYAASSYLSLTPEQWKSHKSYSCQVTHEGS |
| | | | Region | TVEKTVAPTECS |
| | | | Amino Acid | |
| | | | Sequence | |
| 232 | Human Cλ | IGCλ | Cλ Light | ggtcagcccaaggctgccccctcggtcactctgttcccgccctcctctg |
| | constant | 3*04 | Chain | aggagcttcaagccaacaaggccacactggtgtgtctcataagtgactt |
| | region | | Constant | ctacccgggagccgtgacagtggcctggaaggcagatagcagcccc |
| | | | Region | gtcaaggcgggagtggagaccaccacaccctccaaacaaagcaaca |
| | | | Nucleotide | acaagtacgcggccagcagctacctgagcctgacgcctgagcagtgg |
| | | | Sequence | aagtcccacagaagctacagctgccaggtcacgcatgaagggagcac |
| | | | | cgtggagaagacagtggcccctacagaatgttca |
| 233 | | | Cλ Light | GQPKAAPSVTLFPPSSEELQANKATLVCLISDF |
| | | | Chain | YPGAVTVAWKADSSPVKAGVETTTPSKQSN |
| | | | Constant | NKYAASSYLSLTPEQWKSHRSYSCQVTHEGS |
| | | | Region | TVEKTVAPTECS |
| | | | Amino Acid | |
| | | | Sequence | |
| 234 | Human Cλ | IGCλ | Cλ Light | ggtcagcccaaggctgccccatcggtcactctgttcccgccctcctctg |
| | constant | 6*01 | Chain | aggagcttcaagccaacaaggccacactggtgtgcctgatcagtgactt |
| | region | | Constant | ctacccgggagctgtgaaagtggcctggaaggcagatggcagcccc |
| | | | Region | gtcaacacgggagtggagaccaccacaccctccaaacagagcaaca |
| | | | Nucleotide | acaagtacgcggccagcagctacctgagcctgacgcctgagcagtgg |
| | | | Sequence | aagtcccacagaagctacagctgccaggtcacgcatgaagggagcac |
| | | | | cgtggagaagacagtggcccctgcagaatgttca |
| 235 | | | Cλ Light | GQPKAAPSVTLFPPSSEELQANKATLVCLISDF |
| | | | Chain | YPGAVKVAWKADGSPVNTGVETTTPSKQSN |
| | | | Constant | NKYAASSYLSLTPEQWKSHRSYSCQVTHEGS |
| | | | Region | TVEKTVAPAECS |
| | | | Amino Acid | |
| | | | Sequence | |
| 236 | Human Cλ | IGLC | Cλ Light | ggtcagcccaaggctgccccatcggtcactctgttcccaccctcctctg |
| | constant | 7*01 & | Chain | aggagcttcaagccaacaaggccacactggtgtgtctcgtaagtgactt |
| | region | IGCλ | Constant | ctacccgggagccgtgacagtggcctggaaggcagatggcagcccc |
| | | 7*02 | Region | gtcaaggtgggagtggagaccaccaaaccctccaaacaaagcaacaa |
| | | | Nucleotide | caagtatgcggccagcagctacctgagcctgacgcccgagcagtgga |
| | | | Sequence | agtcccacagaagctacagctgccgggtcacgcatgaagggagcacc |
| | | | | gtggagaagacagtggcccctgcagaatgctct |
| 237 | | | Cλ Light | GQPKAAPSVTLFPPSSEELQANKATLVCLVSD |
| | | | Chain | FYPGAVTVAWKADGSPVKVGVETTKPSKQS |
| | | | Constant | NNKYAASSYLSLTPEQWKSHRSYSCRVTHEG |
| | | | Region | STVEKTVAPAECS |
| | | | Amino Acid | |
| | | | Sequence | |
| 238 | 413G05- | Amino acid | | GFTFSDYY |
| | CDRH1 | sequence of CDRH1 | | |
| | (IMGT) | of 413G05 using | | |
| | | IMGT | | |
| 239 | 413G05- | Amino acid | | ISTSGSTI |
| | CDRH2 | sequence of CDRH2 | | |
| | (IMGT) | of 413G05 using | | |
| | | IMGT | | |
| 240 | 413G05- | Amino acid | | ARGITGTNFYHYGLGV |
| | CDRH3 | sequence of CDRH3 | | |
| | (IMGT) | of 413G05 using | | |
| | | IMGT | | |
| 241 | 413G05- | Amino acid | | DYYMS |
| | CDRH1 | sequence of CDRH1 | | |
| | (Kabat) | of 413G05 using | | |
| | | Kabat | | |
| 242 | 413G05- | Amino acid | | YISTSGSTIYYADSVKG |
| | CDRH2 | sequence of CDRH2 | | |
| | (Kabat) | of 413G05 using | | |
| | | Kabat | | |
| 243 | 413G05- | Amino acid | | GITGTNFYHYGLGV |
| | CDRH3 | sequence of CDRH3 | | |
| | (Kabat) | of 413G05 using | | |
| | | Kabat | | |
| 244 | 413G05- | Amino acid | | QVQLVESGGGLVKPGGSLRLSCAASGFTFSD |
| | Heavy | sequence of V_{H} of | | YYMSWIRQVPGKGLEWVSYISTSGSTIYYAD |
| | chain | 413G05 | | SVKGRFTISRDNAKNSLYLQMNSLRAEDAAV |
| | variable | | | YHCARGITGTNFYHYGLGVWGQGTTVTVSS |
| | region | | | |
| 245 | 413G05- | Nucleic acid | | CAGGTGCAGCTGGTGGAGTCTGGGGGAGGC |
| | Heavy | sequence of V_{H} of | | TTGGTCAAGCCTGGAGGGTCCCTGAGACTC |
| | chain | 413G05 | | TCCTGTGCAGCCTCTGGATTCACCTTCAGTG |
| | variable | | | ACTACTACATGAGCTGGATCCGCCAGGTTC |
| | region | | | CAGGGAAGGGGCTGGAGTGGGTTTCATACA |
| | | | | TTAGTACTAGTGGTAGTACCATATACTACG |
| | | | | CAGACTCTGTGAAGGGCCGATTCACCATCT |
| | | | | CCAGGGACAACGCCAAGAACTCACTGTATC |
| | | | | TACAAATGAACAGCCTGAGAGCCGAGGAC |
| | | | | GCGGCCGTGTATCACTGTGCGAGAGGTATA |
| | | | | ACTGGAACTAACTTCTACCACTACGGTTTG |
| | | | | GGCGTCTGGGGCCAAGGGACCACGGTCACC |
| | | | | GTCTCCTCAG |
| 246 | 413G05- | Amino acid | | QVQLVESGGGLVKPGGSLRLSCAASGFTFSD |
| | full | sequence of 413G05 | | YYMSWIRQVPGKGLEWVSYISTSGSTIYYAD |
| | heavy | heavy chain | | SVKGRFTISRDNAKNSLYLQMNSLRAEDAAV |
| | chain | | | YHCARGITGTNFYHYGLGVWGQGTTVTVSS |
| | sequence | | | ASTKGPSVFPLAPSSKSTSGGTAALGCLVKDY |
| | | | | FPEPVTVSWNSGALTSGVHTFPAVLQSSGLYS |
| | | | | LSSVVTVPSSSLGTQTYICNVNHKPSNTKVDK |
| | | | | KVEPKSCDKTHTCPPCPAPELLGGPSVFLFPP |
| | | | | KPKDTLMISRTPEVTCVVVDVSHEDPEVKFN |
| | | | | WYVDGVEVHNAKTKPREEQYNSTYRVVSVL |
| | | | | TVLHQDWLNGKEYKCKVSNKALPAPIEKTIS |
| | | | | KAKGQPREPQVYTLPPSRDELTKNQVSLTCL |
| | | | | VKGFYPSDIAVEWESNGQPENNYKTTPPVLD |
| | | | | SDGSFFLYSKLTVDKSRWQQGNVFSCSVMHE |
| | | | | ALHNHYTQKSLSLSPGK |
| 247 | 413G05- | Nucleic acid | | CAGGTGCAGCTGGTGGAGTCTGGGGGAGGC |
| | full | sequence of 413G05 | | TTGGTCAAGCCTGGAGGGTCCCTGAGACTC |
| | heavy | heavy chain | | TCCTGTGCAGCCTCTGGATTCACCTTCAGTG |
| | chain | | | ACTACTACATGAGCTGGATCCGCCAGGTTC |
| | sequence | | | CAGGGAAGGGGCTGGAGTGGGTTTCATACA |
| | | | | TTAGTACTAGTGGTAGTACCATATACTACG |
| | | | | CAGACTCTGTGAAGGGCCGATTCACCATCT |
| | | | | CCAGGGACAACGCCAAGAACTCACTGTATC |
| | | | | TACAAATGAACAGCCTGAGAGCCGAGGAC |
| | | | | GCGGCCGTGTATCACTGTGCGAGAGGTATA |
| | | | | ACTGGAACTAACTTCTACCACTACGGTTTG |
| | | | | GGCGTCTGGGGCCAAGGGACCACGGTCACC |
| | | | | GTCTCCTCAGCCAGCACCAAGGGCCCCTCT |
| | | | | GTGTTCCCTCTGGCCCCTTCCAGCAAGTCCA |
| | | | | CCTCTGGCGGAACAGCCGCTCTGGGCTGCC |
| | | | | TCGTGAAGGACTACTTCCCCGAGCCTGTGA |
| | | | | CCGTGTCCTGGAACTCTGGCGCTCTGACCA |
| | | | | GCGGAGTGCACACCTTCCCTGCTGTGCTGC |
| | | | | AGTCCTCCGGCCTGTACTCCCTGTCCTCCGT |
| | | | | CGTGACCGTGCCTTCCAGCTCTCTGGGCAC |
| | | | | CCAGACCTACATCTGCAACGTGAACCACAA |
| | | | | GCCCTCCAACACCAAGGTGGACAAGAAGGT |
| | | | | GGAACCCAAGTCCTGCGACAAGACCCACAC |
| | | | | CTGTCCCCCTTGTCCTGCCCCTGAACTGCTG |
| | | | | GGCGGACCTTCCGTGTTCCTGTTCCCCCCAA |
| | | | | AGCCCAAGGACACCCTGATGATCTCCCGGA |
| | | | | CCCCCGAAGTGACCTGCGTGGTGGTGGATG |
| | | | | TGTCCCACGAGGACCCTGAAGTGAAGTTCA |
| | | | | ATTGGTACGTGGACGGCGTGGAAGTGCACA |
| | | | | ACGCCAAGACCAAGCCTAGAGAGGAACAG |
| | | | | TACAACTCCACCTACCGGGTGGTGTCCGTG |
| | | | | CTGACCGTGCTGCACCAGGATTGGCTGAAC |
| | | | | GGCAAAGAGTACAAGTGCAAGGTGTCCAA |
| | | | | CAAGGCCCTGCCTGCCCCCATCGAAAAGAC |
| | | | | CATCTCCAAGGCCAAGGGCCAGCCCCGGGA |
| | | | | ACCCCAGGTGTACACACTGCCCCCTAGCAG |
| | | | | GGACGAGCTGACCAAGAACCAGGTGTCCCT |
| | | | | GACCTGTCTCGTGAAAGGCTTCTACCCCTCC |
| | | | | GATATCGCCGTGGAATGGGAGTCCAACGGC |
| | | | | CAGCCTGAGAACAACTACAAGACCACCCCC |
| | | | | CCTGTGCTGGACTCCGACGGCTCATTCTTCC |
| | | | | TGTACAGCAAGCTGACAGTGGACAAGTCCC |
| | | | | GGTGGCAGCAGGGCAACGTGTTCTCCTGCT |
| | | | | CCGTGATGCACGAGGCCCTGCACAACCACT |
| | | | | ACACCCAGAAGTCCCTGTCCCTGAGCCCCG |
| | | | | GCAAG |
| 248 | 413G05- | Amino acid | | QGINSW |
| | CDRL1 | sequence of CDRL1 | | |
| | (IMGT) | of 413G05 using | | |
| | | IMGT | | |
| 249 | 413G05- | Amino acid | | AAS |
| | CDRL2 | sequence of CDRL2 | | |
| | (IMGT) | of 413G05 using | | |
| | | IMGT | | |
| 250 | 413G05- | Amino acid | | QQVNSFPLT |
| | CDRL3 | sequence of CDRL3 | | |
| | (IMGT) | of 413G05 using | | |
| | | IMGT | | |
| 251 | 413G05- | Amino acid | | RASQGINSWLA |
| | CDRL1 | sequence of CDRL1 | | |
| | (Kabat) | of 413G05 using | | |
| | | Kabat | | |
| 252 | 413G05- | Amino acid | | AASTLQS |
| | CDRL2 | sequence of CDRL2 | | |
| | (Kabat) | of 413G05 using | | |
| | | Kabat | | |
| 253 | 413G05- | Amino acid | | QQVNSFPLT |
| | CDRL3 | sequence of CDRL3 | | |
| | (Kabat) | of 413G05 using | | |
| | | Kabat | | |
| 254 | 413G05- | Amino acid | | DIQMTQSPSSVSASVGDRVTITCRASQGINSW |
| | Light | sequence of V_{L} of | | LAWYQQKPGKAPKLLIYAASTLQSGVPSRFS |
| | chain | 413G05 | | GSGSGADFTLTISSLQPEDFATYYCQQVNSFP |
| | variable | | | LTFGGGTKVEIK |
| | region | | | |
| 255 | 413G05- | Nucleic acid | | GACATCCAGATGACCCAGTCTCCATCTTCC |
| | Light | sequence of V_{L} of | | GTGTCTGCATCTGTAGGAGACAGAGTCACC |
| | chain | 413G05 | | ATCACTTGTCGGGCGAGTCAGGGTATTAAC |
| | variable | | | AGCTGGTTAGCCTGGTATCAGCAGAAACCA |
| | region | | | GGGAAAGCCCCTAAGCTCCTGATCTATGCT |
| | | | | GCATCCACTTTGCAAAGTGGGGTCCCATCA |
| | | | | AGGTTCAGCGGCAGTGGGTCTGGGGCAGAT |
| | | | | TTCACTCTCACCATCAGCAGCCTGCAGCCT |
| | | | | GAAGATTTTGCAACTTACTATTGTCAACAG |
| | | | | GTTAACAGTTTCCCGCTCACTTTCGGCGGA |
| | | | | GGGACCAAGGTGGAGATCAAAC |
| 256 | 413G05- | Amino acid | | DIQMTQSPSSVSASVGDRVTITCRASQGINSW |
| | full | sequence of 413G05 | | LAWYQQKPGKAPKLLIYAASTLQSGVPSRFS |
| | light | light chain | | GSGSGADFTLTISSLQPEDFATYYCQQVNSFP |
| | chain | | | LTFGGGTKVEIKRTVAAPSVFIFPPSDEQLKSG |
| | sequence | | | TASVVCLLNNFYPREAKVQWKVDNALQSGN |
| | | | | SQESVTEQDSKDSTYSLSSTLTLSKADYEKHK |
| | | | | VYACEVTHQGLSSPVTKSFNRGEC |
| 257 | 413G05- | Nucleic acid | | GACATCCAGATGACCCAGTCTCCATCTTCC |
| | full | sequence of 413G05 | | GTGTCTGCATCTGTAGGAGACAGAGTCACC |
| | light | light chain | | ATCACTTGTCGGGCGAGTCAGGGTATTAAC |
| | chain | | | AGCTGGTTAGCCTGGTATCAGCAGAAACCA |
| | sequence | | | GGGAAAGCCCCTAAGCTCCTGATCTATGCT |
| | | | | GCATCCACTTTGCAAAGTGGGGTCCCATCA |
| | | | | AGGTTCAGCGGCAGTGGGTCTGGGGCAGAT |
| | | | | TTCACTCTCACCATCAGCAGCCTGCAGCCT |
| | | | | GAAGATTTTGCAACTTACTATTGTCAACAG |
| | | | | GTTAACAGTTTCCCGCTCACTTTCGGCGGA |
| | | | | GGGACCAAGGTGGAGATCAAACGTACGGT |
| | | | | GGCCGCTCCCTCCGTGTTCATCTTCCCACCT |
| | | | | TCCGACGAGCAGCTGAAGTCCGGCACCGCT |
| | | | | TCTGTCGTGTGCCTGCTGAACAACTTCTACC |
| | | | | CCCGCGAGGCCAAGGTGCAGTGGAAGGTG |
| | | | | GACAACGCCCTGCAGTCCGGCAACTCCCAG |
| | | | | GAATCCGTGACCGAGCAGGACTCCAAGGAC |
| | | | | AGCACCTACTCCCTGTCCTCCACCCTGACCC |
| | | | | TGTCCAAGGCCGACTACGAGAAGCACAAG |
| | | | | GTGTACGCCTGCGAAGTGACCCACCAGGGC |
| | | | | CTGTCTAGCCCCGTGACCAAGTCTTTCAACC |
| | | | | GGGGCGAGTGT |
| 258 | 413F09- | Amino acid | | GFTFSYYA |
| | CDRH1 | sequence of CDRH1 | | |
| | (IMGT) | of 413F09 using | | |
| | | IMGT | | |
| 259 | 413F09- | Amino acid | | ISGGGGNT |
| | CDRH2 | sequence of CDRH2 | | |
| | (IMGT) | of 413F09 using | | |
| | | IMGT | | |
| 260 | 413F09- | Amino acid | | AKDRMKQLVRAYYFDY |
| | CDRH3 | sequence of CDRH3 | | |
| | (IMGT) | of 413F09 using | | |
| | | IMGT | | |
| 261 | 413F09- | Amino acid | | YYAMS |
| | CDRH1 | sequence of CDRH1 | | |
| | (Kabat) | of 413F09 using | | |
| | | Kabat | | |
| 262 | 413F09- | Amino acid | | TISGGGGNTHYADSVKG |
| | CDRH2 | sequence of CDRH2 | | |
| | (Kabat) | of 413F09 using | | |
| | | Kabat | | |
| 263 | 413F09- | Amino acid | | DRMKQLVRAYYFDY |
| | CDRH3 | sequence of CDRH3 | | |
| | (Kabat) | of 413F09 using | | |
| | | Kabat | | |
| 264 | 413F09- | Amino acid | | EVPLVESGGGLVQPGGSLRLSCAASGFTFSYY |
| | Heavy | sequence of V_{H} of | | AMSWVRQAPGKGLDWVSTISGGGGNTHYAD |
| | chain | 413F09 | | SVKGRFTISRDNSKNTLYLHMNSLRAEDTAV |
| | variable | | | YYCAKDRMKQLVRAYYFDYWGQGTLVTVS |
| | region | | | S |
| 265 | 413F09- | Nucleic acid | | GAGGTGCCGCTGGTGGAGTCTGGGGGAGGC |
| | Heavy | sequence of V_{H} of | | TTGGTACAGCCTGGGGGGTCCCTGAGACTC |
| | chain | 413F09 | | TCCTGTGCAGCCTCTGGATTCACGTTTAGCT |
| | variable | | | ACTATGCCATGAGCTGGGTCCGTCAGGCTC |
| | region | | | CAGGGAAGGGGCTGGACTGGGTCTCAACTA |
| | | | | TTAGTGGTGGTGGTGGTAACACACACTACG |
| | | | | CAGACTCCGTGAAGGGCCGATTCACTATAT |
| | | | | CCAGAGACAATTCCAAGAACACGCTGTATC |
| | | | | TGCACATGAACAGCCTGAGAGCCGAAGAC |
| | | | | ACGGCCGTCTATTACTGTGCGAAGGATCGG |
| | | | | ATGAAACAGCTCGTCCGGGCCTACTACTTT |
| | | | | GACTACTGGGGCCAGGGAACCCTGGTCACC |
| | | | | GTCTCCTCAG |
| 266 | 413F09- | Amino acid | | EVPLVESGGGLVQPGGSLRLSCAASGFTFSYY |
| | full | sequence of 413F09 | | AMSWVRQAPGKGLDWVSTISGGGGNTHYAD |
| | heavy | heavy chain | | SVKGRFTISRDNSKNTLYLHMNSLRAEDTAV |
| | chain | | | YYCAKDRMKQLVRAYYFDYWGQGTLVTVS |
| | sequence | | | SASTKGPSVFPLAPSSKSTSGGTAALGCLVKD |
| | | | | YFPEPVTVSWNSGALTSGVHTFPAVLQSSGL |
| | | | | YSLSSWTVPSSSLGTQTYICNVNHKPSNTKV |
| | | | | DKKVEPKSCDKTHTCPPCPAPELLGGPSVFLF |
| | | | | PPKPKDTLMISRTPEVTCVVVDVSHEDPEVKF |
| | | | | NWYVDGVEVHNAKTKPREEQYNSTYRVVSV |
| | | | | LTVLHQDWLNGKEYKCKVSNKALPAPIEKTI |
| | | | | SKAKGQPREPQVYTLPPSRDELTKNQVSLTCL |
| | | | | VKGFYPSDIAVEWESNGQPENNYKTTPPVLD |
| | | | | SDGSFFLYSKLTVDKSRWQQGNVFSCSVMHE |
| | | | | ALHNHYTQKSLSLSPGK |
| 267 | 413F09- | Nucleic acid | | GAGGTGCCGCTGGTGGAGTCTGGGGGAGGC |
| | full | sequence of 413F09 | | TTGGTACAGCCTGGGGGGTCCCTGAGACTC |
| | heavy | heavy chain | | TCCTGTGCAGCCTCTGGATTCACGTTTAGCT |
| | chain | | | ACTATGCCATGAGCTGGGTCCGTCAGGCTC |
| | sequence | | | CAGGGAAGGGGCTGGACTGGGTCTCAACTA |
| | | | | TTAGTGGTGGTGGTGGTAACACACACTACG |
| | | | | CAGACTCCGTGAAGGGCCGATTCACTATAT |
| | | | | CCAGAGACAATTCCAAGAACACGCTGTATC |
| | | | | TGCACATGAACAGCCTGAGAGCCGAAGAC |
| | | | | ACGGCCGTCTATTACTGTGCGAAGGATCGG |
| | | | | ATGAAACAGCTCGTCCGGGCCTACTACTTT |
| | | | | GACTACTGGGGCCAGGGAACCCTGGTCACC |
| | | | | GTCTCCTCAGCCAGCACCAAGGGCCCCTCT |
| | | | | GTGTTCCCTCTGGCCCCTTCCAGCAAGTCCA |
| | | | | CCTCTGGCGGAACAGCCGCTCTGGGCTGCC |
| | | | | TCGTGAAGGACTACTTCCCCGAGCCTGTGA |
| | | | | CCGTGTCCTGGAACTCTGGCGCTCTGACCA |
| | | | | GCGGAGTGCACACCTTCCCTGCTGTGCTGC |
| | | | | AGTCCTCCGGCCTGTACTCCCTGTCCTCCGT |
| | | | | CGTGACCGTGCCTTCCAGCTCTCTGGGCAC |
| | | | | CCAGACCTACATCTGCAACGTGAACCACAA |
| | | | | GCCCTCCAACACCAAGGTGGACAAGAAGGT |
| | | | | GGAACCCAAGTCCTGCGACAAGACCCACAC |
| | | | | CTGTCCCCCTTGTCCTGCCCCTGAACTGCTG |
| | | | | GGCGGACCTTCCGTGTTCCTGTTCCCCCCAA |
| | | | | AGCCCAAGGACACCCTGATGATCTCCCGGA |
| | | | | CCCCCGAAGTGACCTGCGTGGTGGTGGATG |
| | | | | TGTCCCACGAGGACCCTGAAGTGAAGTTCA |
| | | | | ATTGGTACGTGGACGGCGTGGAAGTGCACA |
| | | | | ACGCCAAGACCAAGCCTAGAGAGGAACAG |
| | | | | TACAACTCCACCTACCGGGTGGTGTCCGTG |
| | | | | CTGACCGTGCTGCACCAGGATTGGCTGAAC |
| | | | | GGCAAAGAGTACAAGTGCAAGGTGTCCAA |
| | | | | CAAGGCCCTGCCTGCCCCCATCGAAAAGAC |
| | | | | CATCTCCAAGGCCAAGGGCCAGCCCCGGGA |
| | | | | ACCCCAGGTGTACACACTGCCCCCTAGCAG |
| | | | | GGACGAGCTGACCAAGAACCAGGTGTCCCT |
| | | | | GACCTGTCTCGTGAAAGGCTTCTACCCCTCC |
| | | | | GATATCGCCGTGGAATGGGAGTCCAACGGC |
| | | | | CAGCCTGAGAACAACTACAAGACCACCCCC |
| | | | | CCTGTGCTGGACTCCGACGGCTCATTCTTCC |
| | | | | TGTACAGCAAGCTGACAGTGGACAAGTCCC |
| | | | | GGTGGCAGCAGGGCAACGTGTTCTCCTGCT |
| | | | | CCGTGATGCACGAGGCCCTGCACAACCACT |
| | | | | ACACCCAGAAGTCCCTGTCCCTGAGCCCCG |
| | | | | GCAAG |
| 268 | 413F09- | Amino acid | | QDISTY |
| | CDRL1 | sequence of CDRL1 | | |
| | (IMGT) | of 413F09 using | | |
| | | IMGT | | |
| 269 | 413F09- | Amino acid | | GTS |
| | CDRL2 | sequence of CDRL2 | | |
| | (IMGT) | of 413F09 using | | |
| | | IMGT | | |
| 270 | 413F09- | Amino acid | | QQLHTDPIT |
| | CDRL3 | sequence of CDRL3 | | |
| | (IMGT) | of 413F09 using | | |
| | | IMGT | | |
| 271 | 413F09- | Amino acid | | WASQDISTYLG |
| | CDRL1 | sequence of CDRL1 | | |
| | (Kabat) | of 413F09 using | | |
| | | Kabat | | |
| 272 | 413F09- | Amino acid | | GTSSLQS |
| | CDRL2 | sequence of CDRL2 | | |
| | (Kabat) | of 413F09 using | | |
| | | Kabat | | |
| 273 | 413F09- | Amino acid | | QQLHTDPIT |
| | CDRL3 | sequence of CDRL3 | | |
| | (Kabat) | of 413F09 using | | |
| | | Kabat | | |
| 274 | 413F09- | Amino acid | | DIQLTQSPSFLSASVGDRVTITCWASQDISTYL |
| | Light | sequence of V_{L} of | | GWYQQKPGKAPKLLIYGTSSLQSGVPSRFSGS |
| | chain | 413F09 | | GSGTEFTLTISSLQPEDFATYYCQQLHTDPITF |
| | variable | | | GQGTRLEIK |
| | region | | | |
| 275 | 413F09- | Nucleic acid | | GACATCCAGTTGACCCAGTCTCCATCCTTCC |
| | Light | sequence of V_{L} of | | TGTCTGCATCTGTAGGAGACAGAGTCACCA |
| | chain | 413F09 | | TCACTTGCTGGGCCAGTCAGGACATTAGCA |
| | variable | | | CTTATTTAGGCTGGTATCAGCAAAAACCAG |
| | region | | | GGAAAGCCCCTAAGCTCCTGATCTATGGTA |
| | | | | CATCCAGTTTGCAAAGTGGGGTCCCATCAA |
| | | | | GGTTCAGCGGCAGTGGATCTGGGACAGAAT |
| | | | | TCACTCTCACAATCAGCAGCCTGCAGCCTG |
| | | | | AAGATTTTGCAACTTATTACTGTCAACAGCT |
| | | | | TCATACTGACCCGATCACCTTCGGCCAAGG |
| | | | | GACACGACTGGAGATCAAAC |
| 276 | 413F09- | Amino acid | | DIQLTQSPSFLSASVGDRVTITCWASQDISTYL |
| | full light | sequence of 413F09 | | GWYQQKPGKAPKLLIYGTSSLQSGVPSRFSGS |
| | chain | light chain | | GSGTEFTLTISSLQPEDFATYYCQQLHTDPITF |
| | sequence | | | GQGTRLEIKRTVAAPSVFIFPPSDEQLKSGTAS |
| | | | | VVCLLNNFYPREAKVQWKVDNALQSGNSQE |
| | | | | SVTEQDSKDSTYSLSSTLTLSKADYEKHKVY |
| | | | | ACEVTHQGLSSPVTKSFNRGEC |
| 277 | 413F09- | Nucleic acid | | GACATCCAGTTGACCCAGTCTCCATCCTTCC |
| | full light | sequence of 413F09 | | TGTCTGCATCTGTAGGAGACAGAGTCACCA |
| | chain | light chain | | TCACTTGCTGGGCCAGTCAGGACATTAGCA |
| | sequence | | | CTTATTTAGGCTGGTATCAGCAAAAACCAG |
| | | | | GGAAAGCCCCTAAGCTCCTGATCTATGGTA |
| | | | | CATCCAGTTTGCAAAGTGGGGTCCCATCAA |
| | | | | GGTTCAGCGGCAGTGGATCTGGGACAGAAT |
| | | | | TCACTCTCACAATCAGCAGCCTGCAGCCTG |
| | | | | AAGATTTTGCAACTTATTACTGTCAACAGCT |
| | | | | TCATACTGACCCGATCACCTTCGGCCAAGG |
| | | | | GACACGACTGGAGATCAAACGTACGGTGGC |
| | | | | CGCTCCCTCCGTGTTCATCTTCCCACCTTCC |
| | | | | GACGAGCAGCTGAAGTCCGGCACCGCTTCT |
| | | | | GTCGTGTGCCTGCTGAACAACTTCTACCCCC |
| | | | | GCGAGGCCAAGGTGCAGTGGAAGGTGGAC |
| | | | | AACGCCCTGCAGTCCGGCAACTCCCAGGAA |
| | | | | TCCGTGACCGAGCAGGACTCCAAGGACAGC |
| | | | | ACCTACTCCCTGTCCTCCACCCTGACCCTGT |
| | | | | CCAAGGCCGACTACGAGAAGCACAAGGTG |
| | | | | TACGCCTGCGAAGTGACCCACCAGGGCCTG |
| | | | | TCTAGCCCCGTGACCAAGTCTTTCAACCGG |
| | | | | GGCGAGTGT |
| 278 | 414B06- | Amino acid | | GFTFSSYW |
| | CDRH1 | sequence of CDRH1 | | |
| | (IMGT) | of 414B06 using | | |
| | | IMGT | | |
| 279 | 414B06- | Amino acid | | IKQDGSEK |
| | CDRH2 | sequence of CDRH2 | | |
| | (IMGT) | of 414B06 using | | |
| | | IMGT | | |
| 280 | 414B06- | Amino acid | | ARVRQWSDYSDY |
| | CDRH3 | sequence of CDRH3 | | |
| | (IMGT) | of 414B06 using | | |
| | | IMGT | | |
| 281 | 414B06- | Amino acid | | SYWMN |
| | CDRH1 | sequence of CDRH1 | | |
| | (Kabat) | of 414B06 using | | |
| | | Kabat | | |
| 282 | 414B06- | Amino acid | | NIKQDGSEKYYVDSVKG |
| | CDRH2 | sequence of CDRH2 | | |
| | (Kabat) | of 414B06 using | | |
| | | Kabat | | |
| 283 | 414B06- | Amino acid | | VRQWSDYSDY |
| | CDRH3 | sequence of CDRH3 | | |
| | (Kabat) | of 414B06 using | | |
| | | Kabat | | |
| 284 | 414B06- | Amino acid | | EVHLVESGGGLVQPGGSLRLSCAASGFTFSSY |
| | Heavy | sequence of V_{H} of | | WMNWVRQAPGKGLEWVANIKQDGSEKYYV |
| | chain | 414B06 | | DSVKGRFTVSRDNAKNSLYLQMNSLRAEDT |
| | variable | | | AVYYCARVRQWSDYSDYWGQGTPVTVSS |
| | region | | | |
| 285 | 414B06- | Nucleic acid | | GAGGTGCACCTGGTGGAGTCTGGGGGAGGC |
| | Heavy | sequence of V_{H} of | | TTGGTCCAGCCTGGGGGGTCCCTGAGACTC |
| | chain | 414B06 | | TCCTGTGCAGCCTCTGGATTCACCTTTAGTA |
| | variable | | | GCTATTGGATGAACTGGGTCCGCCAGGCTC |
| | region | | | CAGGGAAGGGGCTGGAGTGGGTGGCCAAC |
| | | | | ATAAAGCAAGATGGAAGTGAGAAATACTA |
| | | | | TGTGGACTCTGTGAAGGGCCGCTTCACCGT |
| | | | | CTCCAGAGACAACGCCAAGAACTCACTGTA |
| | | | | TCTGCAAATGAACAGCCTGAGAGCCGAGGA |
| | | | | CACGGCTGTGTATTACTGTGCGAGAGTTCG |
| | | | | ACAATGGTCCGACTACTCTGACTACTGGGG |
| | | | | CCAGGGAACCCCGGTCACCGTCTCCTCAG |
| 286 | 414B06- | Amino acid | | EVHLVESGGGLVQPGGSLRLSCAASGFTFSSY |
| | full | sequence of 414B06 | | WMNWVRQAPGKGLEWVANIKQDGSEKYYV |
| | heavy | heavy chain | | DSVKGRFTVSRDNAKNSLYLQMNSLRAEDT |
| | chain | | | AVYYCARVRQWSDYSDYWGQGTPVTVSSAS |
| | sequence | | | TKGPSVFPLAPSSKSTSGGTAALGCLVKDYFP |
| | | | | EPVTVSWNSGALTSGVHTFPAVLQSSGLYSLS |
| | | | | SWTVPSSSLGTQTYICNVNHKPSNTKVDKK |
| | | | | VEPKSCDKTHTCPPCPAPELLGGPSVFLFPPKP |
| | | | | KDTLMISRTPEVTCVVVDVSHEDPEVKFNWY |
| | | | | VDGVEVHNAKTKPREEQYNSTYRVVSVLTV |
| | | | | LHQDWLNGKEYKCKVSNKALPAPIEKTISKA |
| | | | | KGQPREPQVYTLPPSRDELTKNQVSLTCLVK |
| | | | | GFYPSDIAVEWESNGQPENNYKTTPPVLDSD |
| | | | | GSFFLYSKLTVDKSRWQQGNVFSCSVMHEAL |
| | | | | HNHYTQKSLSLSPGK |
| 287 | 414B06- | Nucleic acid | | GAGGTGCACCTGGTGGAGTCTGGGGGAGGC |
| | full | sequence of 414B06 | | TTGGTCCAGCCTGGGGGGTCCCTGAGACTC |
| | heavy | heavy chain | | TCCTGTGCAGCCTCTGGATTCACCTTTAGTA |
| | chain | | | GCTATTGGATGAACTGGGTCCGCCAGGCTC |
| | sequence | | | CAGGGAAGGGGCTGGAGTGGGTGGCCAAC |
| | | | | ATAAAGCAAGATGGAAGTGAGAAATACTA |
| | | | | TGTGGACTCTGTGAAGGGCCGCTTCACCGT |
| | | | | CTCCAGAGACAACGCCAAGAACTCACTGTA |
| | | | | TCTGCAAATGAACAGCCTGAGAGCCGAGGA |
| | | | | CACGGCTGTGTATTACTGTGCGAGAGTTCG |
| | | | | ACAATGGTCCGACTACTCTGACTACTGGGG |
| | | | | CCAGGGAACCCCGGTCACCGTCTCCTCAGC |
| | | | | CAGCACCAAGGGCCCCTCTGTGTTCCCTCT |
| | | | | GGCCCCTTCCAGCAAGTCCACCTCTGGCGG |
| | | | | AACAGCCGCTCTGGGCTGCCTCGTGAAGGA |
| | | | | CTACTTCCCCGAGCCTGTGACCGTGTCCTGG |
| | | | | AACTCTGGCGCTCTGACCAGCGGAGTGCAC |
| | | | | ACCTTCCCTGCTGTGCTGCAGTCCTCCGGCC |
| | | | | TGTACTCCCTGTCCTCCGTCGTGACCGTGCC |
| | | | | TTCCAGCTCTCTGGGCACCCAGACCTACAT |
| | | | | CTGCAACGTGAACCACAAGCCCTCCAACAC |
| | | | | CAAGGTGGACAAGAAGGTGGAACCCAAGT |
| | | | | CCTGCGACAAGACCCACACCTGTCCCCCTT |
| | | | | GTCCTGCCCCTGAACTGCTGGGCGGACCTT |
| | | | | CCGTGTTCCTGTTCCCCCCAAAGCCCAAGG |
| | | | | ACACCCTGATGATCTCCCGGACCCCCGAAG |
| | | | | TGACCTGCGTGGTGGTGGATGTGTCCCACG |
| | | | | AGGACCCTGAAGTGAAGTTCAATTGGTACG |
| | | | | TGGACGGCGTGGAAGTGCACAACGCCAAG |
| | | | | ACCAAGCCTAGAGAGGAACAGTACAACTCC |
| | | | | ACCTACCGGGTGGTGTCCGTGCTGACCGTG |
| | | | | CTGCACCAGGATTGGCTGAACGGCAAAGAG |
| | | | | TACAAGTGCAAGGTGTCCAACAAGGCCCTG |
| | | | | CCTGCCCCCATCGAAAAGACCATCTCCAAG |
| | | | | GCCAAGGGCCAGCCCCGGGAACCCCAGGT |
| | | | | GTACACACTGCCCCCTAGCAGGGACGAGCT |
| | | | | GACCAAGAACCAGGTGTCCCTGACCTGTCT |
| | | | | CGTGAAAGGCTTCTACCCCTCCGATATCGC |
| | | | | CGTGGAATGGGAGTCCAACGGCCAGCCTGA |
| | | | | GAACAACTACAAGACCACCCCCCCTGTGCT |
| | | | | GGACTCCGACGGCTCATTCTTCCTGTACAG |
| | | | | CAAGCTGACAGTGGACAAGTCCCGGTGGCA |
| | | | | GCAGGGCAACGTGTTCTCCTGCTCCGTGAT |
| | | | | GCACGAGGCCCTGCACAACCACTACACCCA |
| | | | | GAAGTCCCTGTCCCTGAGCCCCGGCAAG |
| 288 | 414B06- | Amino acid | | QGISSW |
| | CDRL1 | sequence of CDRL1 | | |
| | (IMGT) | of 414B06 using | | |
| | | IMGT | | |
| 289 | 414B06- | Amino acid | | AAS |
| | CDRL2 | sequence of CDRL2 | | |
| | (IMGT) | of 414B06 using | | |
| | | IMGT | | |
| 290 | 414B06- | Amino acid | | QQANSFPFT |
| | CDRL3 | sequence of CDRL3 | | |
| | (IMGT) | of 414B06 using | | |
| | | IMGT | | |
| 291 | 414B06- | Amino acid | | RASQGISSWLA |
| | CDRL1 | sequence of CDRL1 | | |
| | (Kabat) | of 414B06 using | | |
| | | Kabat | | |
| 292 | 414B06- | Amino acid | | AASSLQS |
| | CDRL2 | sequence of CDRL2 | | |
| | (Kabat) | of 414B06 using | | |
| | | Kabat | | |
| 293 | 414B06- | Amino acid | | QQANSFPFT |
| | CDRL3 | sequence of CDRL3 | | |
| | (Kabat) | of 414B06 using | | |
| | | Kabat | | |
| 294 | 414B06- | Amino acid | | DIQMTQSPSSVSASVGDRVTITCRASQGISSW |
| | Light | sequence of V_{L} of | | LAWYQQKPGKAPKLLIYAASSLQSGVPSRFS |
| | chain | 414B06 | | GSGSGTDFTLTISSLQPEDFATYYCQQANSFPF |
| | variable | | | TFGPGTKVDIK |
| | region | | | |
| 295 | 414B06- | Nucleic acid | | GACATCCAGATGACCCAGTCTCCATCTTCC |
| | Light | sequence of V_{L} of | | GTGTCTGCATCTGTAGGAGACAGAGTCACC |
| | chain | 414B06 | | ATCACTTGTCGGGCGAGTCAGGGTATTAGC |
| | variable | | | AGCTGGTTAGCCTGGTATCAGCAGAAACCA |
| | region | | | GGGAAAGCCCCTAAGCTCCTGATCTATGCT |
| | | | | GCATCCAGTTTGCAAAGTGGGGTCCCATCA |
| | | | | AGGTTCAGCGGCAGTGGATCTGGGACAGAT |
| | | | | TTCACTCTCACCATCAGCAGCCTGCAGCCT |
| | | | | GAAGATTTTGCAACTTACTATTGTCAACAG |
| | | | | GCTAACAGTTTCCCATTCACTTTCGGCCCTG |
| | | | | GGACCAAAGTGGATATCAAAC |
| 296 | 414B06- | Amino acid | | DIQMTQSPSSVSASVGDRVTITCRASQGISSW |
| | full | sequence of 414B06 | | LAWYQQKPGKAPKLLIYAASSLQSGVPSRFS |
| | light | light chain | | GSGSGTDFTLTISSLQPEDFATYYCQQANSFPF |
| | chain | | | TFGPGTKVDIKRTVAAPSVFIFPPSDEQLKSGT |
| | sequence | | | ASVVCLLNNFYPREAKVQWKVDNALQSGNS |
| | | | | QESVTEQDSKDSTYSLSSTLTLSKADYEKHKV |
| | | | | YACEVTHQGLSSPVTKSFNRGEC |
| 297 | 414B06- | Nucleic acid | | GACATCCAGATGACCCAGTCTCCATCTTCC |
| | full | sequence of 414B06 | | GTGTCTGCATCTGTAGGAGACAGAGTCACC |
| | light | light chain | | ATCACTTGTCGGGCGAGTCAGGGTATTAGC |
| | chain | | | AGCTGGTTAGCCTGGTATCAGCAGAAACCA |
| | sequence | | | GGGAAAGCCCCTAAGCTCCTGATCTATGCT |
| | | | | GCATCCAGTTTGCAAAGTGGGGTCCCATCA |
| | | | | AGGTTCAGCGGCAGTGGATCTGGGACAGAT |
| | | | | TTCACTCTCACCATCAGCAGCCTGCAGCCT |
| | | | | GAAGATTTTGCAACTTACTATTGTCAACAG |
| | | | | GCTAACAGTTTCCCATTCACTTTCGGCCCTG |
| | | | | GGACCAAAGTGGATATCAAACGTACGGTGG |
| | | | | CCGCTCCCTCCGTGTTCATCTTCCCACCTTC |
| | | | | CGACGAGCAGCTGAAGTCCGGCACCGCTTC |
| | | | | TGTCGTGTGCCTGCTGAACAACTTCTACCCC |
| | | | | CGCGAGGCCAAGGTGCAGTGGAAGGTGGA |
| | | | | CAACGCCCTGCAGTCCGGCAACTCCCAGGA |
| | | | | ATCCGTGACCGAGCAGGACTCCAAGGACAG |
| | | | | CACCTACTCCCTGTCCTCCACCCTGACCCTG |
| | | | | TCCAAGGCCGACTACGAGAAGCACAAGGT |
| | | | | GTACGCCTGCGAAGTGACCCACCAGGGCCT |
| | | | | GTCTAGCCCCGTGACCAAGTCTTTCAACCG |
| | | | | GGGCGAGTGT |
| 298 | Mutated | Amino acid | | DIQMTQSPSSLSASVGDRVTITCRASQSISSYL |
| | 1D05- | sequence of 1D05 | | NWYQQKPGKAPKLLIYYASSLQSGVPSRFSG |
| | LC | kappa light chain | | SGSGTDFTLTISSLQPEDFATYYCQQSYSTPIT |
| | mutant 3 | with V to Y | | FGQGTRLEIKRTVAAPSVFIFPPSDEQLKSGTA |
| | | mutation in CDRL2 | | SVVCLLNNFYPREAKVQWKVDNALQSGNSQ |
| | | highlighted | | ESVTEQDSKDSTYSLSSTLTLSKADYEKHKVY |
| | | | | ACEVTHQGLSSPVTKSFNRGEC |
| 299 | 1D05- | Amino acid | | EVQLVESGGGLVQPGRSLRLSCAASGFTFDD |
| | heavy | sequence of IgG1 | | YAMHWVRQVPGKGLEWVSGISWIRTGIGYA |
| | chain | disabled variant of | | DSVKGRFTIFRDNAKNSLYLQMNSLRAEDTA |
| | disabled | 1D05 | | LYYCAKDMKGSGTYGGWFDTWGQGTLVTV |
| | IgG1 Fc | | | SSASTKGPSVFPLAPSSKSTSGGTAALGCLVK |
| | | | | DYFPEPVTVSWNSGALTSGVHTFPAVLQSSG |
| | | | | LYSLSSVVTVPSSSLGTQTYICNVNHKPSNTK |
| | | | | VDKKVEPKSCDKTHTCPPCPAPELAGAPSVF |
| | | | | LFPPKPKDTLMISRTPEVTCVVVDVSHEDPEV |
| | | | | KFNWYVDGVEVHNAKTKPREEQYNSTYRVV |
| | | | | SVLTVLHQDWLNGKEYKCKVSNKALPAPIEK |
| | | | | TISKAKGQPREPQVYTLPPSRDELTKNQVSLT |
| | | | | CLVKGFYPSDIAVEWESNGQPENNYKTTPPV |
| | | | | LDSDGSFFLYSKLTVDKSRWQQGNVFSCSVM |
| | | | | HEALHNHYTQKSLSLSPGK |
| 300 | 1D05- | 1D05 Light chain | | DIQMTQSPSSLSASVGDRVTITCRASQSISSYL |
| | light | amino acid sequence | | NWYQQKPGKAPKLLIYVASSLQSGVPSRFSG |
| | chain IL- | sequence fused to | | SGSGTDFTLTISSLQPEDFATYYCQQSYSTPIT |
| | 2 fusion | wild-type human IL- | | FGQGTRLEIKRTVAAPSVFIFPPSDEQLKSGTA |
| | | 2 sequence (IL-2 | | SVVCLLNNFYPREAKVQWKVDNALQSGNSQ |
| | | is underlined and | | ESVTEQDSKDSTYSLSSTLTLSKADYEKHKVY |
| | | region to be varied is | | ACEVTHQGLSSPVTKSFNRGECAPTSSSTKK |
| | | shown in bold) | | TQLQLEHLLLDLQMILNGINNYKNPKLTRM |
| | | | | LTFKFYMPKKATELKHLQCLEEELKPLEEVL |
| | | | | NLAQSKNFHLRPRDLISNINVIVLELKGSETTF |
| | | | | MCEYADETATIVEFLNRWITFCQSIISTLT |
| 301 | Human | Uniprot number: | | APTSSSTKKTQLQLEHLLLDLQMILNGINNY |
| | IL-2 | P60568 | | KNPKLTRMLTFKFYMPKKATELKHLQCLEEE |
| | | Full length amino | | LKPLEEVLNLAQSKNFHLRPRDLISNINVIVLE |
| | | acid sequence of | | LKGSETTFMCEYADETATIVEFLNRWITFCQSI |
| | | human IL-2 (minus | | ISTLT |
| | | signal sequence) | | |
| 302 | Control | Heavy chain 1D05 | | EVQLVESGGGLVQPGRSLRLSCAASGFTFDD |
| | 1D05 | IgG1 variant fused at | | YAMHWVRQVPGKGLEWVSGISWIRTGIGYA |
| | immuno- | the N-terminus to | | DSVKGRFTIFRDNAKNSLYLQMNSLRAEDTA |
| | cytokine | wild-type human IL2 | | LYYCAKDMKGSGTYGGWFDTWGQGTLVTV |
| | HC C- | sequence (control) | | SSASTKGPSVFPLAPSSKSTSGGTAALGCLVK |
| | terminal | | | DYFPEPVTVSWNSGALTSGVHTFPAVLQSSG |
| | fusion | | | LYSLSSVVTVPSSSLGTQTYICNVNHKPSNTK |
| | | | | VDKKVEPKSCDKTHTCPPCPAPELAGAPSVF |
| | | | | LFPPKPKDTLMISRTPEVTCVVVDVSHEDPEV |
| | | | | KFNWYVDGVEVHNAKTKPREEQYNSTYRVV |
| | | | | SVLTVLHQDWLNGKEYKCKVSNKALPAPIEK |
| | | | | TISKAKGQPREPQVYTLPPSRDELTKNQVSLT |
| | | | | CLVKGFYPSDIAVEWESNGQPENNYKTTPPV |
| | | | | LDSDGSFFLYSKLTVDKSRWQQGNVFSCSVM |
| | | | | HEALHNHYTQKSLSLSPGKAPTSSSTKKTQLQ |
| | | | | LEHLLLDLQMILNGINNYKNPKLTRMLTFKF |
| | | | | YMPKKATELKHLQCLEEELKPLEEVLNLAQS |
| | | | | KNFHLRPRDLISNINVIVLELKGSETTFMCEYA |
| | | | | DETATIVEFLNRWITFCQSIISTLT |
| 303 | IL-2 D5- | IL-2 IC45 (Del 5-9) | | APTSTQLQLELLLD |
| | 9 | N terminal IL-2 | | |
| | | sequence | | |
| 304 | IL-2D1- | IL-2 IC46 (Del 1-9) | | TQLQLEHLLLD |
| | 9 | N terminal IL-2 | | |
| | | sequence | | |
| 305 | IL-2 D5- | IL-2 IC64 (Del 5-7) | | APTSKKTQLQLEHLLLD |
| | 7 | N terminal IL-2 | | |
| | | sequence | | |
| 306 | IL-2 D1 | IL-2 DIN terminal | | PTSSSTKKTQLQLEHLLLD |
| | | IL-2 sequence | | |
| 307 | IL-2D1- | IL-2D1-2N | | TSSSTKKTQLQLEHLLLD |
| | 2 | terminal IL-2 | | |
| | | sequence | | |
| 308 | IL-2D1- | IL-2 DI-3 N | | SSSTKKTQLQLEHLLLD |
| | 3 | terminal IL-2 | | |
| | | sequence | | |
| 309 | IL-2D1- | IL-2D1-4N | | SSTKKTQLQLEHLLLD |
| | 4 | terminal IL-2 | | |
| | | sequence | | |
| 310 | IL-2D1- | IL-2 DI-5 N | | STKKTQLQLEHLLLD |
| | 5 | terminal IL-2 | | |
| | | sequence | | |
| 311 | IL-2D1- | IL-2D1-6N | | TKKTQLQLEHLLLD |
| | 6 | terminal IL-2 | | |
| | | sequence | | |
| 312 | IL-2D1- | IL-2D1-7N | | KKTQLQLEHLLLD |
| | 7 | terminal IL-2 | | |
| | | sequence | | |
| 313 | IL-2D1- | IL-2D1-8N | | KTQLQLEHLLLD |
| | 8 | terminal IL-2 | | |
| | | sequence | | |
| 314 | IL-2 D9 | IL-2 D9 N terminal | | APTSSSTKTQLQLEHLLLD |
| | | IL-2 sequence | | |
| 315 | IL-2 D9- | IL-2 D9-8 N | | APTSSSTTQLQLEHLLLD |
| | 8 | terminal IL-2 | | |
| | | sequence | | |
| 316 | IL-2 D9- | IL-2 D9-7 N | | APTSSSTQLQLEHLLLD |
| | 7 | terminal IL-2 | | |
| | | sequence | | |
| 317 | IL-2 D9- | IL-2 D9-6 N | | APTSSTQLQLEHLLLD |
| | 6 | terminal IL-2 | | |
| | | sequence | | |
| 318 | IL-2 D9- | IL-2 D9-4 N | | APTTQLQLEHLLLD |
| | 4 | terminal IL-2 | | |
| | | sequence | | |
| 319 | IL-2 D9- | IL-2 D9-3 N | | APTQLQLEHLLLD |
| | 3 | terminal IL-2 | | |
| | | sequence | | |
| 320 | IL-2 D9- | IL-2 D9-2 N | | ATQLQLEHLLLD |
| | 2 | terminal IL-2 | | |
| | | sequence | | |
| 321 | IL-2 D2- | IL-2 D2-6 N | | ATKKTQLQLEHLLLD |
| | 6 | terminal IL-2 | | |
| | | sequence | | |
| 322 | IL-2 D3- | IL-2 D3-7 N | | APKKTQLQLEHLLLD |
| | 7 | terminal IL-2 | | |
| | | sequence | | |
| 323 | IL-2 D4- | IL-2 D4-8 N | | APTKTQLQLEHLLLD |
| | 8 | terminal IL-2 | | |
| | | sequence | | |
| 324 | C- | Amino acids 21 to | | LQMILNGINNYKNPKLTRMLTFKFYMPKKAT |
| | terminal | 133 of hIL-2 | | ELKHLQCLEEELKPLEEVLNLAQSKNFHLRPR |
| | amino | | | DLISNINVIVLELKGSETTFMCEYADETATIVE |
| | acid | | | FLNRWITFCQSIISTLT |
| | sequence | | | |
| | of hIL-2 | | | |
| 325 | Mouse | Uniprot number: | | MRIFAGIIFTACCHLLRAFTITAPKDLYVVEY |
| | PD-L1 | (ECD highlighted in | | GSNVTMECRFPVERELDLLALVVYWEKED |
| | | BOLD, and | | EQVIQFVAGEEDLKPQHSNFRGRASLPKDQ |
| | | cytoplasmic domain | | LLKGNAALQITDVKLQDAGVYCCIISYGGA |
| | | underlined) | | DYKRITLKVNAPYRKINQRISVDPATSEHEL |
| | | Q9EP73 | | ICQAEGYPEAEVIWTNSDHQPVSGKRSVTT |
| | | | | SRTEGMLLNVTSSLRVNATANDVFYCTFW |
| | | | | RSQPGQNHTAELIIPELPATHPPQNRTHWV |
| | | | | LLGSILLFLIVVSTVLLFLRKQVRMLDVEKCG |
| | | | | VEDTSSKNRNDTQFEET |
| 326 | Mouse | Mouse PD-L1 | | FTITAPKDLYVVEYGSNVTMECRFPVERELDL |
| | PD-L1 | extracellular domain | | LALVVYWEKEDEQVIQFVAGEEDLKPQHSNF |
| | ECD His | with his tag | | RGRASLPKDQLLKGNAALQITDVKLQDAGV |
| | | | | YCCIISYGGADYKRITLKVNAPYRKINQRISV |
| | | | | DPATSEHELICQAEGYPEAEVIWTNSDHQPVS |
| | | | | GKRSVTTSRTEGMLLNVTSSLRVNATANDVF |
| | | | | YCTFWRSQPGQNHTAELIIPELPATHPPQNRT |
| | | | | HHHHHH |
| 327 | Human | Human IL-2 receptor | | ELCDDDPPEIPHATFKAMAYKEGTMLNCECK |
| | IL-2Rα | alpha chain | | RGFRRIKSGSLYMLCTGNSSHSSWDNQCQCT |
| | chain | | | SSATRNTTKQVTPQPEEQKERKTTEMQSPMQ |
| | | | | PVDQASLPGHCREPPPWENEATERIYHFVVG |
| | | | | QMVYYQCVQGYRALHRGPAESVCKMTHGK |
| | | | | TRWTQPQLICTGEMETSQFPGEEKPQASPEGR |
| | | | | PESETSCLVTTTDFQIQTEMAATMETSIFTTEY |
| | | | | QVAVAGCVFLLISVLLLSGLTWQRRQRKSRR |
| | | | | TI |
| 328 | Human | Human IL-2 receptor | | AVNGTSQFTCFYNSRANISCVWSQDGALQDT |
| | IL-2Rβ | beta chain | | SCQVHAWPDRRRWNQTCELLPVSQASWACN |
| | chain | | | LILGAPDSQKLTTVDIVTLRVLCREGVRWRV |
| | | | | MAIQDFKPFENLRLMAPISLQVVHVETHRCNI |
| | | | | SWEISQASHYFERHLEFEARTLSPGHTWEEAP |
| | | | | LLTLKQKQEWICLETLTPDTQYEFQVRVKPL |
| | | | | QGEFTTWSPWSQPLAFRTKPAALGKDTIPWL |
| | | | | GHLLVGLSGAFGFIILVYLLINCRNTGPWLKK |
| | | | | VLKCNTPDPSKFFSQLSSEHGGDVQKWLSSPF |
| | | | | PSSSFSPGGLAPEISPLEVLERDKVTQLLLQQD |
| | | | | KVPEPASLSSNHSLTSCFTNQGYFFFHLPDAL |
| | | | | EIEACQVYFTYDPYSEEDPDEGVAGAPTGSSP |
| | | | | QPLQPLSGEDDAYCTFPSRDDLLLFSPSLLGG |
| | | | | PSPPSTAPGGSGAGEERMPPSLQERVPRDWDP |
| | | | | QPLGPPTPGVPDLVDFQPPPELVLREAGEEVP |
| | | | | DAGPREGVSFPWSRPPGQGEFRALNARLPLN |
| | | | | TDAYLSLQELQGQDPTHLV |
| 329 | Human | Human IL-2 receptor | | LNTTILTPNGNEDTTADFFLTTMPTDSLSVSTL |
| | IL-2Rγ | common gamma | | PLPEVQCFVFNVEYMNCTWNSSSEPQPTNLT |
| | chain | chain | | LHYWYKNSDNDKVQKCSHYLFSEEITSGCQL |
| | | | | QKKEIHLYQTFVVQLQDPREPRRQATQMLKL |
| | | | | QNLVIPWAPENLTLHKLSESQLELNWNNRFL |
| | | | | NHCLEHLVQYRTDWDHSWTEQSVDYRHKFS |
| | | | | LPSVDGQKRYTFRVRSRFNPLCGSAQHWSEW |
| | | | | SHPIHWGSNTSKENPFLFALEAVVISVGSMGL |
| | | | | IISLLCVYFWLERTMPRIPTLKNLEDLVTEYH |
| | | | | GNFSAWSGVSKGLAESLQPDYSERLCLVSEIP |
| | | | | PKGGALGEGPGASPCNQHSPYWAPPCYTLKP |
| | | | | ET |
| 330 | IL-7 | Human IL-7 amino | | DCDIEGKDGKQYESVLMVSIDQLLDSMKEIG |
| | | acid sequence | | SNCLNNEFNFFKRHICDANKEGMFLFRAARK |
| | | | | LRQFLKMNSTGDFDLHLLKVSEGTTILLNCTG |
| | | | | QVKGRKPAALGEAQPTKSLEENKSLKEQKKL |
| | | | | NDLCFLKRLLQEIKTCWNKILMGTKEH |
| 331 | IL-15 | Human IL-15 amino | | GIHVFILGCFSAGLPKTEANWVNVISDLKKIE |
| | | acid sequence | | DLIQSMHIDATLYTESDVHPSCKVTAMKCFLL |
| | | | | ELQVISLESGDASIHDTVENLIILANNSLSSNG |
| | | | | NVTESGCKECEELEEKNIKEFLQSFVHIVQMFI |
| | | | | NTS |
| 332 | IL-21 | Human IL-21 amino | | QGQDRHMIRMRQLIDIVDQLKNYVNDLVPEF |
| | | acid sequence | | LPAPEDVETNCEWSAFSCFQKAQLKSANTGN |
| | | | | NERIINVSIKKLKRKPPSTNAGRRQKHRLTCPS |
| | | | | CDSYEKKPPKEFLERFKSLLQKMIHQHLSSRT |
| | | | | HGSEDS |
| 333 | GM-CSF | Human GM-CSF | | APARSPSPSTQPWEHVNAIQEARRLLNLSRDT |
| | | amino acid sequence | | AAEMNETVEVISEMFDLQEPTCLQTRLELYK |
| | | | | QGLRGSLTKLKGPLTMMASHYKQHCPPTPET |
| | | | | SCATQIITFESFKENLKDFLLVIPFDCWEPVQE |
| 334 | IFNα | Human IFN-α | | CDLPQNHGLLSRNTLVLLHQMRRISPFLCLKD |
| | | amino acid sequence | | RRDFRFPQEMVKGSQLQKAHVMSVLHEMLQ |
| | | | | QIFSLFHTERSSAAWNMTLLDQLHTELHQQL |
| | | | | QHLETCLLQVVGEGESAGAISSPALTLRRYFQ |
| | | | | GIRVYLKEKKYSDCAWEVVRMEIMKSLFLST |
| | | | | NMQERLRSKDRDLGS |
| 335 | TNFα | Extracellular portion | | GPQREEFPRDLSLISPLAQAVRSSSRTPSDKPV |
| | | of human TNF-a | | AHVVANPQAEGQLQWLNRRANALLANGVEL |
| | | amino acid sequence | | RDNQLVVPSEGLYLIYSQVLFKGQGCPSTHVL |
| | | | | LTHTISRIAVSYQTKVNLLSAIKSPCQRETPEG |
| | | | | AEAKPWYEPIYLGGVFQLEKGDRLSAEINRPD |
| | | | | YLDFAESGQVYFGIIAL |
| 336 | IL-12α | Alpha chain of | | RNLPVATPDPGMFPCLHHSQNLLRAVSNMLQ |
| | | human IL-12 amino | | KARQTLEFYPCTSEEIDHEDITKDKTSTVEAC |
| | | acid sequence | | LPLELTKNESCLNSRETSFITNGSCLASRKTSF |
| | | | | MMALCLSSIYEDLKMYQVEFKTMNAKLLMD |
| | | | | PKRQIFLDQNMLAVIDELMQALNFNSETVPQ |
| | | | | KSSLEEPDFYKTKIKLCILLHAFRIRAVTIDRV |
| | | | | MSYLNAS |
| 337 | IL-12β | Beta chain of human | | IWELKKDVYVVELDWYPDAPGEMVVLTCDT |
| | | IL-12 amino acid | | PEEDGITWTLDQSSEVLGSGKTLTIQVKEFGD |
| | | sequence | | AGQYTCHKGGEVLSHSLLLLHKKEDGIWSTD |
| | | | | ILKDQKEPKNKTFLRCEAKNYSGRFTCWWLT |
| | | | | TISTDLTFSVKSSRGSSDPQGVTCGAATLSAE |
| | | | | RVRGDNKEYEYSVECQEDSACPAAEESLPIEV |
| | | | | MVDAVHKLKYENYTSSFFIRDIIKPDPPKNLQ |
| | | | | LKPLKNSRQVEVSWEYPDTWSTPHSYFSLTF |
| | | | | CVQVQGKSKREKKDRVFTDKTSATVICRKNA |
| | | | | SISVRAQDRYYSSSWSEWASVPCS |
| 338 | CXCL9 | Human CXCL-9 | | TPVVRKGRCSCISTNQGTIHLQSLKDLKQFAP |
| | | amino acid sequence | | SPSCEKIEIIATLKNGVQTCLNPDSADVKELIK |
| | | | | KWEKQVSQKKKQKNGKKHQKKKVLKVRKS |
| | | | | QRSRQKKTT |
| 339 | CXCL10 | Human CXCL-10 | | VPLSRTVRCTCISISNQPVNPRSLEKLEIIPASQ |
| | | amino acid sequence | | FCPRVEIIATMKKKGEKRCLNPESKAIKNLLK |
| | | | | AVSKERSKRSP |
| 340 | Human | IGHG | WT human | ASTKGPSVFPLAPSSKSTSGGTAALGCLVKDY |
| | WT | 1*01 | IgG1 amino | FPEPVTVSWNSGALTSGVHTFPAVLQSSGLYS |
| | IgG1 | & | acid | LSSVVTVPSSSLGTQTYICNVNHKPSNTKVDK |
| | constant | IGHG | sequence | KVEPKSCDKTHTCPPCPAPELLGGPSVFLFPP |
| | region | 1*02 | | KPKDTLMISRTPEVTCVVVDVSHEDPEVKFN |
| | | & | | WYVDGVEVHNAKTKPREEQYNSTYRVVSVL |
| | | IGHG | | TVLHQDWLNGKEYKCKVSNKALPAPIEKTIS |
| | | 1*05 | | KAKGQPREPQVYTLPPSRDELTKNQVSLTCL |
| | | (IgG1) | | VKGFYPSDIAVEWESNGQPENNYKTTPPVLD |
| | | | | SDGSFFLYSKLTVDKSRWQQGNVFSCSVMHE |
| | | | | ALHNHYTQKSLSLSPGK |
| 341 | | | WT human | GCCAGCACCAAGGGCCCCTCTGTGTTCCCT |
| | | | IgG1 | CTGGCCCCTTCCAGCAAGTCCACCTCTGGC |
| | | | nucleic acid | GGAACAGCCGCTCTGGGCTGCCTCGTGAAG |
| | | | sequence | GACTACTTCCCCGAGCCTGTGACCGTGTCCT |
| | | | | GGAACTCTGGCGCTCTGACCAGCGGAGTGC |
| | | | | ACACCTTCCCTGCTGTGCTGCAGTCCTCCGG |
| | | | | CCTGTACTCCCTGTCCTCCGTCGTGACCGTG |
| | | | | CCTTCCAGCTCTCTGGGCACCCAGACCTAC |
| | | | | ATCTGCAACGTGAACCACAAGCCCTCCAAC |
| | | | | ACCAAGGTGGACAAGAAGGTGGAACCCAA |
| | | | | GTCCTGCGACAAGACCCACACCTGTCCCCC |
| | | | | TTGTCCTGCCCCTGAACTGCTGGGCGGACC |
| | | | | TTCCGTGTTCCTGTTCCCCCCAAAGCCCAAG |
| | | | | GACACCCTGATGATCTCCCGGACCCCCGAA |
| | | | | GTGACCTGCGTGGTGGTGGATGTGTCCCAC |
| | | | | GAGGACCCTGAAGTGAAGTTCAATTGGTAC |
| | | | | GTGGACGGCGTGGAAGTGCACAACGCCAA |
| | | | | GACCAAGCCTAGAGAGGAACAGTACAACT |
| | | | | CCACCTACCGGGTGGTGTCCGTGCTGACCG |
| | | | | TGCTGCACCAGGATTGGCTGAACGGCAAAG |
| | | | | AGTACAAGTGCAAGGTGTCCAACAAGGCCC |
| | | | | TGCCTGCCCCCATCGAAAAGACCATCTCCA |
| | | | | AGGCCAAGGGCCAGCCCCGGGAACCCCAG |
| | | | | GTGTACACACTGCCCCCTAGCAGGGACGAG |
| | | | | CTGACCAAGAACCAGGTGTCCCTGACCTGT |
| | | | | CTCGTGAAAGGCTTCTACCCCTCCGATATC |
| | | | | GCCGTGGAATGGGAGTCCAACGGCCAGCCT |
| | | | | GAGAACAACTACAAGACCACCCCCCCTGTG |
| | | | | CTGGACTCCGACGGCTCATTCTTCCTGTACA |
| | | | | GCAAGCTGACAGTGGACAAGTCCCGGTGGC |
| | | | | AGCAGGGCAACGTGTTCTCCTGCTCCGTGA |
| | | | | TGCACGAGGCCCTGCACAACCACTACACCC |
| | | | | AGAAGTCCCTGTCCCTGAGCCCCGGCAAGT |
| | | | | GATGA |
| 342 | Mutated | | Amino Acid | EVQLVESGGGLVQPGRSLRLSCAASGFTFDD |
| | 1D05- | | sequence of 1D05 | YAMHWVRQAPGKGLEWVSGISWIRTGIGYA |
| | HC | | heavy chain | DSVKGRFTISRDNAKNSLYLQMNSLRAEDTA |
| | mutant 2 | | with V to A | LYYCAKDMKGSGTYGGWFDTWGQGTLVTV |
| | | | and F to S | SSASTKGPSVFPLAPCSRSTSESTAALGCLVK |
| | | | back-mutation | DYFPEPVTVSWNSGALTSGVHTFPAVLQSSG |
| | | | in framework | LYSLSSVVTVPSSSLGTKTYTCNVDHKPSNTK |
| | | | region to | VDKRVESKYGPPCPPCPAPELAGAPSVFLFPP |
| | | | germline | KPKDTLMISRTPEVTCVVVDVSQEDPEVQFN |
| | | | highlighted | WYVDGVEVHNAKTKPREEQFNSTYRVVSVL |
| | | | with IgG1 | TVLHQDWLNGKEYKCKVSNKGLPSSIEKTIS |
| | | | disabled | KAKGQPREPQVYTLPPSQEEMTKNQVSLTCL |
| | | | (LAGA) | VKGFYPSDIAVEWESNGQPENNYKTTPPVLD |
| | | | constant | SDGSFFLYSRLTVDKSRWQEGNVFSCSVMHE |
| | | | region | ALHNHYTQKSLSLSLGK |

TABLE S2
1.21.1.12. SEQ ID NOS: 343-538

| SEQ | | | |
|---|---|---|---|
| ID | | | |
| NO: | Name | Description | Sequence |
| 343 | 416E01- | Amino acid | GFTFSNYA |
| | CDRH1 | sequence of CDRH1 | |
| | (IMGT) | of 416E01 using | |
| | | IMGT | |
| 344 | 416E01- | Amino acid | ISFSGGTT |
| | CDRH2 | sequence of CDRH2 | |
| | (IMGT) | of 416E01 using | |
| | | IMGT | |
| 345 | 416E01- | Amino acid | AKDEAPAGATFFDS |
| | CDRH3 | sequence of CDRH3 | |
| | (IMGT) | of 416E01 using | |
| | | IMGT | |
| 346 | 416E01- | Amino acid | NYAMS |
| | CDRH1 | sequence of CDRH1 | |
| | (Kabat) | of 416E01 using | |
| | | Kabat | |
| 347 | 416E01- | Amino acid | AISFSGGTTYYADSVKG |
| | CDRH2 | sequence of CDRH2 | |
| | (Kabat) | of 416E01 using | |
| | | Kabat | |
| 348 | 416E01- | Amino acid | DEAPAGATFFDS |
| | CDRH3 | sequence of CDRH3 | |
| | (Kabat) | of 416E01 using | |
| | | Kabat | |
| 349 | 416E01- | Amino acid | EVQLAESGGGLVQPGGSLRLSCAASGFTFSN |
| | Heavy | sequence of V_{H} of | YAMSWVRQTPGKGLEWVSAISFSGGTTYY |
| | chain | 416E01 (mutations | ADSVKGRFTISRDNSKNTLYLHMNSLRADD |
| | variable | from germline are | TAVYYCAKDEAPAGATFFDSWGQGTLVTV |
| | region | shown in bold | SS |
| | | letters) | |
| 350 | 416E01- | Nucleic acid | GAAGTGCAACTGGCGGAGTCTGGGGGAG |
| | Heavy | sequence of V_{H} of | GCTTGGTACAGCCGGGGGGGTCCCTGAGA |
| | chain | 416E01 | CTCTCCTGTGCAGCCTCTGGATTCACCTTT |
| | variable | | AGCAACTATGCCATGAGTTGGGTCCGCCA |
| | region | | GACTCCAGGAAAGGGGCTGGAGTGGGTCT |
| | | | CAGCTATTAGTTTTAGTGGTGGTACTACAT |
| | | | ACTACGCTGACTCCGTGAAGGGCCGGTTC |
| | | | ACCATCTCCAGAGACAATTCCAAGAACAC |
| | | | GCTGTATTTGCACATGAACAGCCTGAGAG |
| | | | CCGATGACACGGCCGTATATTACTGTGCG |
| | | | AAAGATGAGGCACCAGCTGGCGCAACCTT |
| | | | CTTTGACTCCTGGGGCCAGGGAACGCTGG |
| | | | TCACCGTCTCCTCAG |
| 351 | 416E01- | Amino acid | EVQLAESGGGLVQPGGSLRLSCAASGFTFSN |
| | full | sequence of 416E01 | YAMSWVRQTPGKGLEWVSAISFSGGTTYY |
| | heavy | heavy chain | ADSVKGRFTISRDNSKNTLYLHMNSLRADD |
| | chain | | TAVYYCAKDEAPAGATFFDSWGQGTLVTV |
| | sequence | | SSASTKGPSVFPLAPCSRSTSESTAALGCLVK |
| | | | DYFPEPVTVSWNSGALTSGVHTFPAVLQSS |
| | | | GLYSLSSWTVPSSSLGTKTYTCNVDHKPSN |
| | | | TKVDKRVESKYGPPCPPCPAPEFEGGPSVFL |
| | | | FPPKPKDTLMISRTPEVTCVVVDVSQEDPEV |
| | | | QFNWYVDGVEVHNAKTKPREEQFNSTYRV |
| | | | VSVLTVLHQDWLNGKEYKCKVSNKGLPSSI |
| | | | EKTISKAKGQPREPQVYTLPPSQEEMTKNQV |
| | | | SLTCLVKGFYPSDIAVEWESNGQPENNYKT |
| | | | TPPVLDSDGSFFLYSRLTVDKSRWQEGNVFS |
| | | | CSVMHEALHNHYTQKSLSLSLGK |
| 352 | 416E01- | Nucleic acid | GAAGTGCAACTGGCGGAGTCTGGGGGAG |
| | full | sequence of 416E01 | GCTTGGTACAGCCGGGGGGGTCCCTGAGA |
| | heavy | heavy chain | CTCTCCTGTGCAGCCTCTGGATTCACCTTT |
| | chain | | AGCAACTATGCCATGAGTTGGGTCCGCCA |
| | sequence | | GACTCCAGGAAAGGGGCTGGAGTGGGTCT |
| | | | CAGCTATTAGTTTTAGTGGTGGTACTACAT |
| | | | ACTACGCTGACTCCGTGAAGGGCCGGTTC |
| | | | ACCATCTCCAGAGACAATTCCAAGAACAC |
| | | | GCTGTATTTGCACATGAACAGCCTGAGAG |
| | | | CCGATGACACGGCCGTATATTACTGTGCG |
| | | | AAAGATGAGGCACCAGCTGGCGCAACCTT |
| | | | CTTTGACTCCTGGGGCCAGGGAACGCTGG |
| | | | TCACCGTCTCCTCAGCCAGCACCAAGGGC |
| | | | CCTTCCGTGTTCCCCCTGGCCCCTTGCAGC |
| | | | AGGAGCACCTCCGAATCCACAGCTGCCCT |
| | | | GGGCTGTCTGGTGAAGGACTACTTTCCCG |
| | | | AGCCCGTGACCGTGAGCTGGAACAGCGGC |
| | | | GCTCTGACATCCGGCGTCCACACCTTTCCT |
| | | | GCCGTCCTGCAGTCCTCCGGCCTCTACTCC |
| | | | CTGTCCTCCGTGGTGACCGTGCCTAGCTCC |
| | | | TCCCTCGGCACCAAGACCTACACCTGTAA |
| | | | CGTGGACCACAAACCCTCCAACACCAAGG |
| | | | TGGACAAACGGGTCGAGAGCAAGTACGG |
| | | | CCCTCCCTGCCCTCCTTGTCCTGCCCCCGA |
| | | | GTTCGAAGGCGGACCCAGCGTGTTCCTGT |
| | | | TCCCTCCTAAGCCCAAGGACACCCTCATG |
| | | | ATCAGCCGGACACCCGAGGTGACCTGCGT |
| | | | GGTGGTGGATGTGAGCCAGGAGGACCCTG |
| | | | AGGTCCAGTTCAACTGGTATGTGGATGGC |
| | | | GTGGAGGTGCACAACGCCAAGACAAAGC |
| | | | CCCGGGAAGAGCAGTTCAACTCCACCTAC |
| | | | AGGGTGGTCAGCGTGCTGACCGTGCTGCA |
| | | | TCAGGACTGGCTGAACGGCAAGGAGTACA |
| | | | AGTGCAAGGTCAGCAATAAGGGACTGCCC |
| | | | AGCAGCATCGAGAAGACCATCTCCAAGGC |
| | | | TAAAGGCCAGCCCCGGGAACCTCAGGTGT |
| | | | ACACCCTGCCTCCCAGCCAGGAGGAGATG |
| | | | ACCAAGAACCAGGTGAGCCTGACCTGCCT |
| | | | GGTGAAGGGATTCTACCCTTCCGACATCG |
| | | | CCGTGGAGTGGGAGTCCAACGGCCAGCCC |
| | | | GAGAACAATTATAAGACCACCCCTCCCGT |
| | | | CCTCGACAGCGACGGATCCTTCTTTCTGTA |
| | | | CTCCAGGCTGACCGTGGATAAGTCCAGGT |
| | | | GGCAGGAAGGCAACGTGTTCAGCTGCTCC |
| | | | GTGATGCACGAGGCCCTGCACAATCACTA |
| | | | CACCCAGAAGTCCCTGAGCCTGTCCCTGG |
| | | | GAAAG |
| 353 | 416E01- | Amino acid | QGIRRW |
| | CDRL1 | sequence of CDRL1 | |
| | (IMGT) | of 416E01 using | |
| | | IMGT | |
| 354 | 416E01- | Amino acid | GAS |
| | CDRL2 | sequence of CDRL2 | |
| | (IMGT) | of 416E01 using | |
| | | IMGT | |
| 355 | 416E01- | Amino acid | QQANSFPIT |
| | CDRL3 | sequence of CDRL3 | |
| | (IMGT) | of 416E01 using | |
| | | IMGT | |
| 356 | 416E01- | Amino acid | RASQGIRRWLA |
| | CDRL1 | sequence of CDRL1 | |
| | (Kabat) | of 416E01 using | |
| | | Kabat | |
| 357 | 416E01- | Amino acid | GASSLQS |
| | CDRL2 | sequence of CDRL2 | |
| | (Kabat) | of 416E01 using | |
| | | Kabat | |
| 358 | 416E01- | Amino acid | QQANSFPIT |
| | CDRL3 | sequence of CDRL3 | |
| | (Kabat) | of 416E01 using | |
| | | Kabat | |
| 359 | 416E01- | Amino acid | DIQMTQSPSSVSASVGDRVTITCRASQGIRR |
| | Light | sequence of V_{L} of | WLAWYQQKPGKAPKLLISGASSLQSGVPSR |
| | chain | 416E01 (mutations | FSGSGSGTDFTLIITSLQPEDFATYYCQQA |
| | variable | from germline are | NSFPITFGQGTRLEIK |
| | region | shown in bold | |
| | | letters) | |
| 360 | 416E01- | Nucleic acid | GACATCCAGATGACCCAGTCTCCATCTTCC |
| | Light | sequence of V_{L} of | GTGTCTGCATCTGTAGGAGACAGAGTCAC |
| | chain | 416E01 | CATCACTTGTCGGGCGAGTCAGGGTATTA |
| | variable | | GGAGGTGGTTAGCCTGGTATCAGCAGAAA |
| | region | | CCAGGGAAAGCCCCTAAACTCCTGATCTC |
| | | | TGGTGCATCCAGTTTGCAAAGTGGGGTCC |
| | | | CATCAAGGTTCAGCGGCAGTGGATCTGGG |
| | | | ACAGATTTCACTCTCATCATTACCAGTCTG |
| | | | CAGCCTGAAGATTTTGCAACTTACTATTGT |
| | | | CAACAGGCTAACAGTTTCCCGATCACCTT |
| | | | CGGCCAAGGGACACGACTGGAGATCAAA |
| | | | C |
| 361 | 416E01- | Amino acid | DIQMTQSPSSVSASVGDRVTITCRASQGIRR |
| | full | sequence of 416E01 | WLAWYQQKPGKAPKLLISGASSLQSGVPSRF |
| | light | light chain | SGSGSGTDFTLIITSLQPEDFATYYCQQANS |
| | chain | | FPITFGQGTRLEIKRTVAAPSVFIFPPSDEQ |
| | sequence | | LKSGTASVVCLLNNFYPREAKVQWKVDNALQ |
| | | | SGNSQESVTEQDSKDSTYSLSSTLTLSKADY |
| | | | EKHKVYACEVTHQGLSSPVTKSFNRGEC |
| 362 | 416E01- | Nucleic acid | GACATCCAGATGACCCAGTCTCCATCTTCC |
| | full | sequence of 416E01 | GTGTCTGCATCTGTAGGAGACAGAGTCAC |
| | light | light chain | CATCACTTGTCGGGCGAGTCAGGGTATTA |
| | chain | | GGAGGTGGTTAGCCTGGTATCAGCAGAAA |
| | sequence | | CCAGGGAAAGCCCCTAAACTCCTGATCTC |
| | | | TGGTGCATCCAGTTTGCAAAGTGGGGTCC |
| | | | CATCAAGGTTCAGCGGCAGTGGATCTGGG |
| | | | ACAGATTTCACTCTCATCATTACCAGTCTG |
| | | | CAGCCTGAAGATTTTGCAACTTACTATTGT |
| | | | CAACAGGCTAACAGTTTCCCGATCACCTT |
| | | | CGGCCAAGGGACACGACTGGAGATCAAA |
| | | | CGTACGGTGGCCGCTCCCTCCGTGTTCATC |
| | | | TTCCCACCTTCCGACGAGCAGCTGAAGTC |
| | | | CGGCACCGCTTCTGTCGTGTGCCTGCTGAA |
| | | | CAACTTCTACCCCCGCGAGGCCAAGGTGC |
| | | | AGTGGAAGGTGGACAACGCCCTGCAGTCC |
| | | | GGCAACTCCCAGGAATCCGTGACCGAGCA |
| | | | GGACTCCAAGGACAGCACCTACTCCCTGT |
| | | | CCTCCACCCTGACCCTGTCCAAGGCCGAC |
| | | | TACGAGAAGCACAAGGTGTACGCCTGCGA |
| | | | AGTGACCCACCAGGGCCTGTCTAGCCCCG |
| | | | TGACCAAGTCTTTCAACCGGGGCGAGTGT |
| 363 | STIM001- | Amino acid | GYTFSTFG |
| | CDRH1 | sequence of CDRH1 | |
| | | of STIM001 using | |
| | | IMGT | |
| 364 | STIM001- | Amino acid | ISAYNGDT |
| | CDRH2 | sequence of CDRH2 | |
| | | of STIM001 using | |
| | | IMGT | |
| 365 | STIM001- | Amino acid | ARSSGHYYYYGMDV |
| | CDRH3 | sequence of CDRH3 | |
| | | of STIM001 using | |
| | | IMGT | |
| 366 | STIM001- | Amino acid | QVQVVQSGAEVKKPGASVKVSCKASGYTFS |
| | Heavy | sequence of V_{H} of | TFGITWVRQAPGQGLEWMGWISAYNGDTN |
| | chain | STIM001 | YAQNLQGRVIMTTDTSTSTAYMELRSLRSD |
| | variable | | DTAVYYCARSSGHYYYYGMDVWGQGTTV |
| | region | | TVSS |
| 367 | STIM001- | Nucleic acid | CAGGTTCAGGTGGTGCAGTCTGGAGCTGA |
| | Heavy | sequence of V_{H} of | GGTGAAGAAGCCTGGGGCCTCAGTGAAGG |
| | chain | STIM001 | TCTCCTGCAAGGCTTCTGGTTACACCTTTT |
| | variable | | CCACCTTTGGTATCACCTGGGTGCGACAG |
| | region | | GCCCCTGGACAAGGGCTTGAATGGATGGG |
| | | | ATGGATCAGCGCTTACAATGGTGACACAA |
| | | | ACTATGCACAGAATCTCCAGGGCAGAGTC |
| | | | ATCATGACCACAGACACATCCACGAGCAC |
| | | | AGCCTACATGGAGCTGAGGAGCCTGAGAT |
| | | | CTGACGACACGGCCGTTTATTACTGTGCG |
| | | | AGGAGCAGTGGCCACTACTACTACTACGG |
| | | | TATGGACGTCTGGGGCCAAGGGACCACGG |
| | | | TCACCGTCTCCTCA |
| 368 | STIM001- | Amino acid | QVQVVQSGAEVKKPGASVKVSCKASGYTFS |
| | full | sequence of | TFGITWVRQAPGQGLEWMGWISAYNGDTN |
| | heavy | STIM001 heavy | YAQNLQGRVIMTTDTSTSTAYMELRSLRSD |
| | chain | chain | DTAVYYCARSSGHYYYYGMDVWGQGTTVTV |
| | sequence | | SSASTKGPSVFPLAPSSKSTSGGTAALGC |
| | | | LVKDYFPEPVTVSWNSGALTSGVHTFPAVL |
| | | | QSSGLYSLSSVVTVPSSSLGTQTYICNVNHK |
| | | | PSNTKVDKKVEPKSCDKTHTCPPCPAPELLG |
| | | | GPSVFLFPPKPKDTLMISRTPEVTCVVVDVS |
| | | | HEDPEVKFNWYVDGVEVHNAKTKPREEQY |
| | | | NSTYRVVSVLTVLHQDWLNGKEYKCKVSNKA |
| | | | LPAPIEKTISKAKGQPREPQVYTLPPSRDE |
| | | | LTKNQVSLTCLVKGFYPSDIAVEWESNGQP |
| | | | ENNYKTTPPVLDSDGSFFLYSKLTVDKSRW |
| | | | QQGNVFSCSVMHEALHNHYTQKSLSLSPGK |
| 369 | STIM001- | Nucleic acid | CAGGTTCAGGTGGTGCAGTCTGGAGCTGA |
| | full | sequence of | GGTGAAGAAGCCTGGGGCCTCAGTGAAGG |
| | heavy | STIM001 heavy | TCTCCTGCAAGGCTTCTGGTTACACCTTTT |
| | chain | chain | CCACCTTTGGTATCACCTGGGTGCGACAG |
| | sequence | | GCCCCTGGACAAGGGCTTGAATGGATGGG |
| | | | ATGGATCAGCGCTTACAATGGTGACACAA |
| | | | ACTATGCACAGAATCTCCAGGGCAGAGTC |
| | | | ATCATGACCACAGACACATCCACGAGCAC |
| | | | AGCCTACATGGAGCTGAGGAGCCTGAGAT |
| | | | CTGACGACACGGCCGTTTATTACTGTGCG |
| | | | AGGAGCAGTGGCCACTACTACTACTACGG |
| | | | TATGGACGTCTGGGGCCAAGGGACCACGG |
| | | | TCACCGTCTCCTCAGCCAGCACCAAGGGC |
| | | | CCCTCTGTGTTCCCTCTGGCCCCTTCCAGC |
| | | | AAGTCCACCTCTGGCGGAACAGCCGCTCT |
| | | | GGGCTGCCTCGTGAAGGACTACTTCCCCG |
| | | | AGCCTGTGACCGTGTCCTGGAACTCTGGC |
| | | | GCTCTGACCAGCGGAGTGCACACCTTCCC |
| | | | TGCTGTGCTGCAGTCCTCCGGCCTGTACTC |
| | | | CCTGTCCTCCGTCGTGACCGTGCCTTCCAG |
| | | | CTCTCTGGGCACCCAGACCTACATCTGCA |
| | | | ACGTGAACCACAAGCCCTCCAACACCAAG |
| | | | GTGGACAAGAAGGTGGAACCCAAGTCCTG |
| | | | CGACAAGACCCACACCTGTCCCCCTTGTC |
| | | | CTGCCCCTGAACTGCTGGGCGGACCTTCC |
| | | | GTGTTCCTGTTCCCCCCAAAGCCCAAGGA |
| | | | CACCCTGATGATCTCCCGGACCCCCGAAG |
| | | | TGACCTGCGTGGTGGTGGATGTGTCCCAC |
| | | | GAGGACCCTGAAGTGAAGTTCAATTGGTA |
| | | | CGTGGACGGCGTGGAAGTGCACAACGCCA |
| | | | AGACCAAGCCTAGAGAGGAACAGTACAA |
| | | | CTCCACCTACCGGGTGGTGTCCGTGCTGA |
| | | | CCGTGCTGCACCAGGATTGGCTGAACGGC |
| | | | AAAGAGTACAAGTGCAAGGTGTCCAACAA |
| | | | GGCCCTGCCTGCCCCCATCGAAAAGACCA |
| | | | TCTCCAAGGCCAAGGGCCAGCCCCGGGAA |
| | | | CCCCAGGTGTACACACTGCCCCCTAGCAG |
| | | | GGACGAGCTGACCAAGAACCAGGTGTCCC |
| | | | TGACCTGTCTCGTGAAAGGCTTCTACCCCT |
| | | | CCGATATCGCCGTGGAATGGGAGTCCAAC |
| | | | GGCCAGCCTGAGAACAACTACAAGACCAC |
| | | | CCCCCCTGTGCTGGACTCCGACGGCTCATT |
| | | | CTTCCTGTACAGCAAGCTGACAGTGGACA |
| | | | AGTCCCGGTGGCAGCAGGGCAACGTGTTC |
| | | | TCCTGCTCCGTGATGCACGAGGCCCTGCA |
| | | | CAACCACTACACCCAGAAGTCCCTGTCCC |
| | | | TGAGCCCCGGCAAGTGATGA |
| 370 | STIM001- | Amino acid | QSLLHSNEYNY |
| | CDRL1 | sequence of CDRL1 | |
| | | of STIM001 using | |
| | | IMGT | |
| 371 | STIM001- | Amino acid | LGS |
| | CDRL2 | sequence of CDRL2 | |
| | | of STIM001 using | |
| | | IMGT | |
| 372 | STIM001- | Amino acid | MQSLQTPLT |
| | CDRL3 | sequence of CDRL3 | |
| | | of STIM001 using | |
| | | IMGT | |
| 373 | STIM001- | Amino acid | DIVMTQSPLSLPVTPGEPASISCRSSQSLLH |
| | Light | sequence of V_{L} of | SNEYNYLDWYLQKPGQSPQLLIFLGSNRASG |
| | chain | STIM001 | VPDRFSGSGSGTDFTLKITRVEAEDVGIYYC |
| | variable | | MQSLQTPLTFGGGTKVEIK |
| | region | | |
| 374 | STIM001- | Nucleic acid | GATATTGTGATGACTCAGTCTCCACTCTCC |
| | Light | sequence of V_{L} of | CTGCCCGTCACCCCTGGAGAGCCGGCCTC |
| | chain | STIM001 | CATCTCCTGCAGGTCTAGTCAGAGCCTCCT |
| | variable | | GCATAGTAATGAATACAACTATTTGGATT |
| | region | | GGTACCTGCAGAAGCCAGGGCAGTCTCCA |
| | | | CAGCTCCTGATCTTTTTGGGTTCTAATCGG |
| | | | GCCTCCGGGGTCCCTGACAGGTTCAGTGG |
| | | | CAGTGGATCAGGCACAGATTTTACACTGA |
| | | | AAATCACCAGAGTGGAGGCTGAGGATGTT |
| | | | GGAATTTATTACTGCATGCAATCTCTACAA |
| | | | ACTCCGCTCACTTTCGGCGGAGGGACCAA |
| | | | GGTGGAGATCAAA |
| 375 | STIM001- | Amino acid | DIVMTQSPLSLPVTPGEPASISCRSSQSLLH |
| | full | sequence of | SNEYNYLDWYLQKPGQSPQLLIFLGSNRASG |
| | light | STIM001 light chain | VPDRFSGSGSGTDFTLKITRVEAEDVGIYYC |
| | chain | | MQSLQTPLTFGGGTKVEIK |
| | sequence | | RTVAAPSVFIFPPSDEQLKSGTASVVCLLNN |
| | | | FYPREAKVQWKVDNALQSGNSQESVTEQD |
| | | | SKDSTYSLSSTLTLSKADYEKHKVYACEVT |
| | | | HQGLSSPVTKSFNRGEC |
| 376 | STIM001- | Nucleic acid | GATATTGTGATGACTCAGTCTCCACTCTCC |
| | full | sequence of | CTGCCCGTCACCCCTGGAGAGCCGGCCTC |
| | light | STIM001 light chain | CATCTCCTGCAGGTCTAGTCAGAGCCTCCT |
| | chain | | GCATAGTAATGAATACAACTATTTGGATT |
| | sequence | | GGTACCTGCAGAAGCCAGGGCAGTCTCCA |
| | | | CAGCTCCTGATCTTTTTGGGTTCTAATCGG |
| | | | GCCTCCGGGGTCCCTGACAGGTTCAGTGG |
| | | | CAGTGGATCAGGCACAGATTTTACACTGA |
| | | | AAATCACCAGAGTGGAGGCTGAGGATGTT |
| | | | GGAATTTATTACTGCATGCAATCTCTACAA |
| | | | ACTCCGCTCACTTTCGGCGGAGGGACCAA |
| | | | GGTGGAGATCAAAcgtacggtggccgctcc |
| | | | cctccgtgttatcttcccaccttccgacga |
| | | | gcagctgaagtccggcaccgcttctgtcgt |
| | | | gtgcctgctgaacaacttctacccccgcga |
| | | | ggccaaggtgcagtggaaggtggacaacgc |
| | | | cctgcagtccggcaactcccaggaatccgt |
| | | | gaccgagcaggactccaaggacagcaccta |
| | | | ctccctgtcctccaccctgaccctgtccaa |
| | | | ggccgactacgagaagcacaaggtgtacgc |
| | | | ctgcgaagtgacccaccagggcctgtctag |
| | | | ccccgtgaccaagtctttcaaccggggcga |
| | | | gtgt |
| 377 | STIM002- | Amino acid | GYTFTSYG |
| | CDRH1 | sequence of CDRH1 | |
| | | of STIM002 using | |
| | | IMGT | |
| 378 | STIM002- | Amino acid | ISAYNGNT |
| | CDRH2 | sequence of CDRH2 | |
| | | of STIM002 using | |
| | | IMGT | |
| 379 | STIM002- | Amino acid | ARSTYFYGSGTLYGMDV |
| | CDRH3 | sequence of CDRH3 | |
| | | of STIM002 using | |
| | | IMGT | |
| 380 | STIM002- | Amino acid | QVQLVQSGGEVKKPGASVKVSCKASGYTFT |
| | Heavy | sequence of V_{H} of | SYGFSWVRQAPGQGLEWMGWISAYNGNTN |
| | chain | STIM002 | YAQKLQGRVTMTTDTSTSTAYMELRSLRSD |
| | variable | | DTAVYYCARSTYFYGSGTLYGMDVWGQGT |
| | region | | TVTVSS |
| 381 | STIM002- | Nucleic acid | CAGGTTCAACTGGTGCAGTCTGGAGGTGA |
| | Heavy | sequence of V_{H} of | GGTGAAGAAGCCTGGGGCCTCAGTGAAGG |
| | chain | STIM002 | TCTCCTGCAAGGCTTCTGGTTACACCTTTA |
| | variable | | CCAGCTATGGTTTCAGCTGGGTGCGACAG |
| | region | | GCCCCTGGACAAGGACTAGAGTGGATGGG |
| | | | ATGGATCAGCGCTTACAATGGTAACACAA |
| | | | ACTATGCACAGAAGCTCCAGGGCAGAGTC |
| | | | ACCATGACCACAGACACATCCACGAGCAC |
| | | | AGCCTACATGGAGCTGAGGAGCTTGAGAT |
| | | | CTGACGACACGGCCGTGTATTACTGTGCG |
| | | | AGATCTACGTATTTCTATGGTTCGGGGACC |
| | | | CTCTACGGTATGGACGTCTGGGGCCAAGG |
| | | | GACCACGGTCACCGTCTCCTCA |
| 382 | STIM002- | Amino acid | QVQLVQSGGEVKKPGASVKVSCKASGYTFT |
| | full | sequence of | SYGFSWVRQAPGQGLEWMGWISAYNGNTN |
| | heavy | STIM002 heavy | YAQKLQGRVTMTTDTSTSTAYMELRSLRSD |
| | chain | chain | DTAVYYCARSTYFYGSGTLYGMDVWGQGT |
| | sequence | | TVTVSSASTKGPSVFPLAPSSKSTSGGTAAL |
| | | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 383 | STIM002- | Nucleic acid | CAGGTTCAACTGGTGCAGTCTGGAGGTGA |
| | full | sequence of | GGTGAAGAAGCCTGGGGCCTCAGTGAAGG |
| | heavy | STIM002 heavy | TCTCCTGCAAGGCTTCTGGTTACACCTTTA |
| | chain | chain | CCAGCTATGGTTTCAGCTGGGTGCGACAG |
| | sequence | | GCCCCTGGACAAGGACTAGAGTGGATGGG |
| | | | ATGGATCAGCGCTTACAATGGTAACACAA |
| | | | ACTATGCACAGAAGCTCCAGGGCAGAGTC |
| | | | ACCATGACCACAGACACATCCACGAGCAC |
| | | | AGCCTACATGGAGCTGAGGAGCTTGAGAT |
| | | | CTGACGACACGGCCGTGTATTACTGTGCG |
| | | | AGATCTACGTATTTCTATGGTTCGGGGACC |
| | | | CTCTACGGTATGGACGTCTGGGGCCAAGG |
| | | | GACCACGGTCACCGTCTCCTCA |
| | | | GCCAGCACCAAGGGCCCCTCTGTGTTCCC |
| | | | TCTGGCCCCTTCCAGCAAGTCCACCTCTGG |
| | | | CGGAACAGCCGCTCTGGGCTGCCTCGTGA |
| | | | AGGACTACTTCCCCGAGCCTGTGACCGTG |
| | | | TCCTGGAACTCTGGCGCTCTGACCAGCGG |
| | | | AGTGCACACCTTCCCTGCTGTGCTGCAGTC |
| | | | CTCCGGCCTGTACTCCCTGTCCTCCGTCGT |
| | | | GACCGTGCCTTCCAGCTCTCTGGGCACCC |
| | | | AGACCTACATCTGCAACGTGAACCACAAG |
| | | | CCCTCCAACACCAAGGTGGACAAGAAGGT |
| | | | GGAACCCAAGTCCTGCGACAAGACCCACA |
| | | | CCTGTCCCCCTTGTCCTGCCCCTGAACTGC |
| | | | TGGGCGGACCTTCCGTGTTCCTGTTCCCCC |
| | | | CAAAGCCCAAGGACACCCTGATGATCTCC |
| | | | CGGACCCCCGAAGTGACCTGCGTGGTGGT |
| | | | GGATGTGTCCCACGAGGACCCTGAAGTGA |
| | | | AGTTCAATTGGTACGTGGACGGCGTGGAA |
| | | | GTGCACAACGCCAAGACCAAGCCTAGAGA |
| | | | GGAACAGTACAACTCCACCTACCGGGTGG |
| | | | TGTCCGTGCTGACCGTGCTGCACCAGGAT |
| | | | TGGCTGAACGGCAAAGAGTACAAGTGCAA |
| | | | GGTGTCCAACAAGGCCCTGCCTGCCCCCA |
| | | | TCGAAAAGACCATCTCCAAGGCCAAGGGC |
| | | | CAGCCCCGGGAACCCCAGGTGTACACACT |
| | | | GCCCCCTAGCAGGGACGAGCTGACCAAGA |
| | | | ACCAGGTGTCCCTGACCTGTCTCGTGAAA |
| | | | GGCTTCTACCCCTCCGATATCGCCGTGGA |
| | | | ATGGGAGTCCAACGGCCAGCCTGAGAACA |
| | | | ACTACAAGACCACCCCCCCTGTGCTGGAC |
| | | | TCCGACGGCTCATTCTTCCTGTACAGCAAG |
| | | | CTGACAGTGGACAAGTCCCGGTGGCAGCA |
| | | | GGGCAACGTGTTCTCCTGCTCCGTGATGC |
| | | | ACGAGGCCCTGCACAACCACTACACCCAG |
| | | | AAGTCCCTGTCCCTGAGCCCCGGCAAGTG |
| | | | ATGA |
| 384 | STIM002- | Amino acid | QSLLHSDGYNY |
| | CDRL1 | sequence of CDRL1 | |
| | | of STIM002 using | |
| | | IMGT | |
| 385 | STIM002- | Amino acid | LGS |
| | CDRL2 | sequence of CDRL2 | |
| | | of STIM002 using | |
| | | IMGT | |
| 386 | STIM002- | Amino acid | MQALQTPLS |
| | CDRL3 | sequence of CDRL3 | |
| | | of STIM002 using | |
| | | IMGT | |
| 387 | STIM002- | Amino acid | DIVMTQSPLSLPVTPGEPASISCRSSQSLLHS |
| | Light | sequence of V_{L} of | DGYNYLDWYLQKPGQSPQLLIYLGSTRASG |
| | chain | STIM002 | FPDRFSGSGSGTDFTLKISRVEAEDVGVYYC |
| | variable | | MQALQTPLSFGQGTKLEIK |
| | region | | |
| 388 | STIM002- | Nucleic acid | GATATTGTGATGACTCAGTCTCCACTCTCC |
| | Light | sequence of V_{L} of | CTGCCCGTCACCCCTGGAGAGCCGGCCTC |
| | chain | STIM002 | CATCTCCTGCAGGTCTAGTCAGAGCCTCCT |
| | variable | | GCATAGTGATGGATACAACTGTTTGGATT |
| | region | | GGTACCTGCAGAAGCCAGGGCAGTCTCCA |
| | | | CAGCTCCTGATCTATTTGGGTTCTACTCGG |
| | | | GCCTCCGGGTTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCGTGCAGTTTTGGCCAGGGGACCA |
| | | | AGCTGGAGATCAAA |
| 389 | STIM002- | Amino acid | DIVMTQSPLSLPVTPGEPASISCRSSQSLLHS |
| | full | sequence of | DGYNYLDWYLQKPGQSPQLLIYLGSTRASG |
| | light | STIM002 light chain | FPDRFSGSGSGTDFTLKISRVEAEDVGVYYC |
| | chain | | MQALQTPLSFGQGTKLEIK |
| | sequence | | RTVAAPSVFIFPPSDEQLKSGTASVVCLLNN |
| | | | FYPREAKVQWKVDNALQSGNSQESVTEQD |
| | | | SKDSTYSLSSTLTLSKADYEKHKVYACEVT |
| | | | HQGLSSPVTKSFNRGEC |
| 390 | STIM002- | Nucleic acid | GATATTGTGATGACTCAGTCTCCACTCTCC |
| | full | sequence of | CTGCCCGTCACCCCTGGAGAGCCGGCCTC |
| | light | STIM002 light chain | CATCTCCTGCAGGTCTAGTCAGAGCCTCCT |
| | chain | | GCATAGTGATGGATACAACTGTTTGGATT |
| | sequence | | GGTACCTGCAGAAGCCAGGGCAGTCTCCA |
| | | | CAGCTCCTGATCTATTTGGGTTCTACTCGG |
| | | | GCCTCCGGGTTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCGTGCAGTTTTGGCCAGGGGACCA |
| | | | AGCTGGAGATCAAAcgtacggtggccgctcc |
| | | | ctccgtgttcatcttcccaccttccgacgag |
| | | | cagctgaagtccggcaccgcttctgtcgtgt |
| | | | gcctgctgaacaacttctacccccgcgaggcc |
| | | | aaggtgcagtggaaggtggacaacgccctgca |
| | | | gtccggcaactcccaggaatccgtgaccgagc |
| | | | aggactccaaggacagcacctactccctgtcc |
| | | | tccaccctgaccctgtccaaggccgactacga |
| | | | gaagcacaaggtgtacgcctgcgaagtgaccc |
| | | | accagggcctgtctagccccgtgaccaagtct |
| | | | ttcaaccggggcgagtgt |
| 391 | STIM002- | Amino acid | GYTFTSYG |
| | B- | sequence of CDRH1 | |
| | CDRH1 | of STIM002-B using | |
| | | IMGT | |
| 392 | STIM002- | Amino acid | ISAYNGNT |
| | B- | sequence of CDRH2 | |
| | CDRH2 | of STIM002-B using | |
| | | IMGT | |
| 393 | STIM002- | Amino acid | ARSTYFYGSGTLYGMDV |
| | B- | sequence of CDRH3 | |
| | CDRH3 | of STIM002-B using | |
| | | IMGT | |
| 394 | STIM002- | Amino acid | QVQLVQSGGEVKKPGASVKVSCKASGYTFT |
| | B- | sequence of V_{H} of | SYGFSWVRQAPGQGLEWMGWISAYNGNTN |
| | Heavy | STIM002-B | YAQKLQGRVTMTTDTSTSTAYMELRSLRSD |
| | chain | | DTAVYYCARSTYFYGSGTLYGMDVWGQGT |
| | variable | | TVTVSS |
| | region | | |
| 395 | STIM002- | Nucleic acid | CAGGTTCAACTGGTGCAGTCTGGAGGTGA |
| | B- | sequence of V_{H} of | GGTGAAGAAGCCTGGGGCCTCAGTGAAGG |
| | Heavy | STIM002-B | TCTCCTGCAAGGCTTCTGGTTACACCTTTA |
| | chain | | CCAGCTATGGTTTCAGCTGGGTGCGACAG |
| | variable | | GCCCCTGGACAAGGACTAGAGTGGATGGG |
| | region | | ATGGATCAGCGCTTACAATGGTAACACAA |
| | | | ACTATGCACAGAAGCTCCAGGGCAGAGTC |
| | | | ACCATGACCACAGACACATCCACGAGCAC |
| | | | AGCCTACATGGAGCTGAGGAGCTTGAGAT |
| | | | CTGACGACACGGCCGTGTATTACTGTGCG |
| | | | AGATCTACGTATTTCTATGGTTCGGGGACC |
| | | | CTCTACGGTATGGACGTCTGGGGCCAAGG |
| | | | GACCACGGTCACCGTCTCCTCA |
| 396 | STIM002- | Amino acid | QVQLVQSGGEVKKPGASVKVSCKASGYTFT |
| | B- | sequence of | SYGFSWVRQAPGQGLEWMGWISAYNGNTN |
| | full | STIM002-B heavy | YAQKLQGRVTMTTDTSTSTAYMELRSLRSD |
| | heavy | chain | DTAVYYCARSTYFYGSGTLYGMDVWGQGT |
| | chain | | TVTVSSASTKGPSVFPLAPSSKSTSGGTAAL |
| | sequence | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 397 | STIM002- | Nucleic acid | CAGGTTCAACTGGTGCAGTCTGGAGGTGA |
| | B- | sequence of | GGTGAAGAAGCCTGGGGCCTCAGTGAAGG |
| | full | STIM002-B heavy | TCTCCTGCAAGGCTTCTGGTTACACCTTTA |
| | heavy | chain | CCAGCTATGGTTTCAGCTGGGTGCGACAG |
| | chain | | GCCCCTGGACAAGGACTAGAGTGGATGGG |
| | sequence | | ATGGATCAGCGCTTACAATGGTAACACAA |
| | | | ACTATGCACAGAAGCTCCAGGGCAGAGTC |
| | | | ACCATGACCACAGACACATCCACGAGCAC |
| | | | AGCCTACATGGAGCTGAGGAGCTTGAGAT |
| | | | CTGACGACACGGCCGTGTATTACTGTGCG |
| | | | AGATCTACGTATTTCTATGGTTCGGGGACC |
| | | | CTCTACGGTATGGACGTCTGGGGCCAAGG |
| | | | GACCACGGTCACCGTCTCCTCAGCCAGCA |
| | | | CCAAGGGCCCCTCTGTGTTCCCTCTGGCCC |
| | | | CTTCCAGCAAGTCCACCTCTGGCGGAACA |
| | | | GCCGCTCTGGGCTGCCTCGTGAAGGACTA |
| | | | CTTCCCCGAGCCTGTGACCGTGTCCTGGA |
| | | | ACTCTGGCGCTCTGACCAGCGGAGTGCAC |
| | | | ACCTTCCCTGCTGTGCTGCAGTCCTCCGGC |
| | | | CTGTACTCCCTGTCCTCCGTCGTGACCGTG |
| | | | CCTTCCAGCTCTCTGGGCACCCAGACCTAC |
| | | | ATCTGCAACGTGAACCACAAGCCCTCCAA |
| | | | CACCAAGGTGGACAAGAAGGTGGAACCC |
| | | | AAGTCCTGCGACAAGACCCACACCTGTCC |
| | | | CCCTTGTCCTGCCCCTGAACTGCTGGGCGG |
| | | | ACCTTCCGTGTTCCTGTTCCCCCCAAAGCC |
| | | | CAAGGACACCCTGATGATCTCCCGGACCC |
| | | | CCGAAGTGACCTGCGTGGTGGTGGATGTG |
| | | | TCCCACGAGGACCCTGAAGTGAAGTTCAA |
| | | | TTGGTACGTGGACGGCGTGGAAGTGCACA |
| | | | ACGCCAAGACCAAGCCTAGAGAGGAACA |
| | | | GTACAACTCCACCTACCGGGTGGTGTCCG |
| | | | TGCTGACCGTGCTGCACCAGGATTGGCTG |
| | | | AACGGCAAAGAGTACAAGTGCAAGGTGTC |
| | | | CAACAAGGCCCTGCCTGCCCCCATCGAAA |
| | | | AGACCATCTCCAAGGCCAAGGGCCAGCCC |
| | | | CGGGAACCCCAGGTGTACACACTGCCCCC |
| | | | TAGCAGGGACGAGCTGACCAAGAACCAG |
| | | | GTGTCCCTGACCTGTCTCGTGAAAGGCTTC |
| | | | TACCCCTCCGATATCGCCGTGGAATGGGA |
| | | | GTCCAACGGCCAGCCTGAGAACAACTACA |
| | | | AGACCACCCCCCCTGTGCTGGACTCCGAC |
| | | | GGCTCATTCTTCCTGTACAGCAAGCTGAC |
| | | | AGTGGACAAGTCCCGGTGGCAGCAGGGCA |
| | | | ACGTGTTCTCCTGCTCCGTGATGCACGAG |
| | | | GCCCTGCACAACCACTACACCCAGAAGTC |
| | | | CCTGTCCCTGAGCCCCGGCAAGTGATGA |
| 398 | STIM002- | Amino acid | QSLLHSDGYNC |
| | B- | sequence of CDRL1 | |
| | CDRL1 | of STIM002-B using | |
| | | IMGT | |
| 399 | STIM002- | Amino acid | LGS |
| | B- | sequence of CDRL2 | |
| | CDRL2 | of STIM002-B using | |
| | | IMGT | |
| 400 | STIM002- | Amino acid | MQALQTPCS |
| | B- | sequence of CDRL3 | |
| | CDRL3 | of STIM002-B using | |
| | | IMGT | |
| 401 | STIM002- | Amino acid | DIVMTQSPLSLPVTPGEPASISCRSSQSLLHS |
| | B- | sequence of V_{L} of | DGYNCLDWYLQKPGQSPQLLIYLGSTRASG |
| | Light | STIM002-B | FPDRFSGSGSGTDFTLKISRVEAEDVGVYYC |
| | chain | | MQALQTPCSFGQGTKLEIK |
| | variable | | |
| | region | | |
| 402 | STIM002- | Nucleic acid | GATATTGTGATGACTCAGTCTCCACTCTCC |
| | B- | sequence of V_{L} of | CTGCCCGTCACCCCTGGAGAGCCGGCCTC |
| | Light | STIM002-B | CATCTCCTGCAGGTCTAGTCAGAGCCTCCT |
| | chain | | GCATAGTGATGGATACAACTGTTTGGATT |
| | variable | | GGTACCTGCAGAAGCCAGGGCAGTCTCCA |
| | region | | CAGCTCCTGATCTATTTGGGTTCTACTCGG |
| | | | GCCTCCGGGTTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCGTGCAGTTTTGGCCAGGGGACCA |
| | | | AGCTGGAGATCAAA |
| 403 | STIM002- | Amino acid | DIVMTQSPLSLPVTPGEPASISCRSSQSLLHS |
| | B- | sequence of | DGYNCLDWYLQKPGQSPQLLIYLGSTRASG |
| | full light | STIM002-B light | FPDRFSGSGSGTDFTLKISRVEAEDVGVYYC |
| | chain | chain | MQALQTPCSFGQGTKLEIK |
| | sequence | | RTVAAPSVFIFPPSDEQLKSGTASVVCLLNN |
| | | | FYPREAKVQWKVDNALQSGNSQESVTEQD |
| | | | SKDSTYSLSSTLTLSKADYEKHKVYACEVT |
| | | | HQGLSSPVTKSFNRGEC |
| 404 | STIM002- | Nucleic acid | GATATTGTGATGACTCAGTCTCCACTCTCC |
| | B- | sequence of | CTGCCCGTCACCCCTGGAGAGCCGGCCTC |
| | full light | STIM002-B light | CATCTCCTGCAGGTCTAGTCAGAGCCTCCT |
| | chain | chain | GCATAGTGATGGATACAACTGTTTGGATT |
| | sequence | | GGTACCTGCAGAAGCCAGGGCAGTCTCCA |
| | | | CAGCTCCTGATCTATTTGGGTTCTACTCGG |
| | | | GCCTCCGGGTTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCGTGCAGTTTTGGCCAGGGGACCA |
| | | | AGCTGGAGATCAAAcgtacggtggccgctc |
| | | | cctccgtgttcatcttcccaccttccgacg |
| | | | agcagctgaagtccggcaccgcttctgtcg |
| | | | tgtgcctgctgaacaacttctacccccgcg |
| | | | aggccaaggtgcagtggaaggtggacaacg |
| | | | ccctgcagtccggcaactcccaggaatccg |
| | | | tgaccgagcaggactccaaggacagcacct |
| | | | actccctgtcctccaccctgaccctgtcca |
| | | | aggccgactacgagaagcacaaggtgtacg |
| | | | cctgcgaagtgacccaccagggcctgtcta |
| | | | gccccgtgaccaagtctttcaaccggggcg |
| | | | agtgt |
| 405 | STIM003 | Amino acid | GVTFDDYG |
| | (KY1044)- | sequence of CDRH1 | |
| | CDRH1 | of STIM003 using | |
| | | IMGT | |
| 406 | STIM003 | Amino acid | INWNGGDT |
| | (KY1044)- | sequence of CDRH2 | |
| | CDRH2 | of STIM003 using | |
| | | IMGT | |
| 407 | STIM003 | Amino acid | ARDFYGSGSYYHVPFDY |
| | (KY1044)- | sequence of CDRH3 | |
| | CDRH3 | of STIM003 using | |
| | | IMGT | |
| 408 | STIM003 | Amino acid | EVQLVESGGGVVRPGGSLRLSCVASGVTFD |
| | (KY1044)- | sequence of V_{H} of | DYGMSWVRQAPGKGLEWVSGINWNGGDT |
| | Heavy | STIM003 | DYSDSVKGRFTISRDNAKNSLYLQMNSLRA |
| | chain | | EDTALYYCARDFYGSGSYYHVPFDYWGQGI |
| | variable | | LVTVSS |
| | region | | |
| 409 | STIM003- | Nucleic acid | GAGGTGCAGCTGGTGGAGTCTGGGGGAGG |
| | Heavy | sequence of V_{H} of | TGTGGTACGGCCTGGGGGGTCCCTGAGAC |
| | chain | STIM003 | TCTCCTGTGTAGCCTCTGGAGTCACCTTTG |
| | variable | | ATGATTATGGCATGAGCTGGGTCCGCCAA |
| | region | | GCTCCAGGGAAGGGGCTGGARTGGGTCTC |
| | | | TGGTATTAATTGGAATGGTGGCGACACAG |
| | | | ATTATTCAGACTCTGTGAAGGGCCGATTC |
| | | | ACCATCTCCAGAGACAACGCCAAGAACTC |
| | | | CCTGTATCTACAAATGAATAGTCTGAGAG |
| | | | CCGAGGACACGGCCTTGTATTACTGTGCG |
| | | | AGGGATTTCTATGGTTCGGGGAGTTATTAT |
| | | | CACGTTCCTTTTGACTACTGGGGCCAGGG |
| | | | AATCCTGGTCACCGTCTCCTCA |
| 410 | STIM003 | Amino acid | EVQLVESGGGVVRPGGSLRLSCVASGVTFD |
| | (KY1044)- | sequence of | DYGMSWVRQAPGKGLEWVSGINWNGGDT |
| | full | STIM003 heavy | DYSDSVKGRFTISRDNAKNSLYLQMNSLRA |
| | heavy | chain | EDTALYYCARDFYGSGSYYHVPFDYWGQGI |
| | chain | | LVTVSSASTKGPSVFPLAPSSKSTSGGTAAL |
| | sequence | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 411 | STIM003- | Nucleic acid | GAGGTGCAGCTGGTGGAGTCTGGGGGAGG |
| | full | sequence of | TGTGGTACGGCCTGGGGGGTCCCTGAGAC |
| | heavy | STIM003 heavy | TCTCCTGTGTAGCCTCTGGAGTCACCTTTG |
| | chain | chain | ATGATTATGGCATGAGCTGGGTCCGCCAA |
| | sequence | | GCTCCAGGGAAGGGGCTGGARTGGGTCTC |
| | | | TGGTATTAATTGGAATGGTGGCGACACAG |
| | | | ATTATTCAGACTCTGTGAAGGGCCGATTC |
| | | | ACCATCTCCAGAGACAACGCCAAGAACTC |
| | | | CCTGTATCTACAAATGAATAGTCTGAGAG |
| | | | CCGAGGACACGGCCTTGTATTACTGTGCG |
| | | | AGGGATTTCTATGGTTCGGGGAGTTATTAT |
| | | | CACGTTCCTTTTGACTACTGGGGCCAGGG |
| | | | AATCCTGGTCACCGTCTCCTCAGCCAGCA |
| | | | CCAAGGGCCCCTCTGTGTTCCCTCTGGCCC |
| | | | CTTCCAGCAAGTCCACCTCTGGCGGAACA |
| | | | GCCGCTCTGGGCTGCCTCGTGAAGGACTA |
| | | | CTTCCCCGAGCCTGTGACCGTGTCCTGGA |
| | | | ACTCTGGCGCTCTGACCAGCGGAGTGCAC |
| | | | ACCTTCCCTGCTGTGCTGCAGTCCTCCGGC |
| | | | CTGTACTCCCTGTCCTCCGTCGTGACCGTG |
| | | | CCTTCCAGCTCTCTGGGCACCCAGACCTAC |
| | | | ATCTGCAACGTGAACCACAAGCCCTCCAA |
| | | | CACCAAGGTGGACAAGAAGGTGGAACCC |
| | | | AAGTCCTGCGACAAGACCCACACCTGTCC |
| | | | CCCTTGTCCTGCCCCTGAACTGCTGGGCGG |
| | | | ACCTTCCGTGTTCCTGTTCCCCCCAAAGCC |
| | | | CAAGGACACCCTGATGATCTCCCGGACCC |
| | | | CCGAAGTGACCTGCGTGGTGGTGGATGTG |
| | | | TCCCACGAGGACCCTGAAGTGAAGTTCAA |
| | | | TTGGTACGTGGACGGCGTGGAAGTGCACA |
| | | | ACGCCAAGACCAAGCCTAGAGAGGAACA |
| | | | GTACAACTCCACCTACCGGGTGGTGTCCG |
| | | | TGCTGACCGTGCTGCACCAGGATTGGCTG |
| | | | AACGGCAAAGAGTACAAGTGCAAGGTGTC |
| | | | CAACAAGGCCCTGCCTGCCCCCATCGAAA |
| | | | AGACCATCTCCAAGGCCAAGGGCCAGCCC |
| | | | CGGGAACCCCAGGTGTACACACTGCCCCC |
| | | | TAGCAGGGACGAGCTGACCAAGAACCAG |
| | | | GTGTCCCTGACCTGTCTCGTGAAAGGCTTC |
| | | | TACCCCTCCGATATCGCCGTGGAATGGGA |
| | | | GTCCAACGGCCAGCCTGAGAACAACTACA |
| | | | AGACCACCCCCCCTGTGCTGGACTCCGAC |
| | | | GGCTCATTCTTCCTGTACAGCAAGCTGAC |
| | | | AGTGGACAAGTCCCGGTGGCAGCAGGGCA |
| | | | ACGTGTTCTCCTGCTCCGTGATGCACGAG |
| | | | GCCCTGCACAACCACTACACCCAGAAGTC |
| | | | CCTGTCCCTGAGCCCCGGCAAGTGATGA |
| 412 | STIM003 | Amino acid | QSVSRSY |
| | (KY1044)- | sequence of CDRL1 | |
| | CDRL1 | of STIM003 using | |
| | | IMGT | |
| 413 | STIM003 | Amino acid | GAS |
| | (KY1044)- | sequence of CDRL2 | |
| | CDRL2 | of STIM003 using | |
| | | IMGT | |
| 414 | STIM003 | Amino acid | HQYDMSPFT |
| | (KY1044)- | sequence of CDRL3 | |
| | CDRL3 | of STIM003 using | |
| | | IMGT | |
| 415 | STIM003 | Amino acid | EIVLTQSPGTLSLSPGERATLSCRASQSVSRS |
| | (KY1044)- | sequence of V_{L} of | YLAWYQQKRGQAPRLLIYGASSRATGIPDR |
| | Light | STIM003 | FSGDGSGTDFTLSISRLEPEDFAVYYCHQYD |
| | chain | | MSPFTFGPGTKVDIK |
| | variable | | |
| | region | | |
| 416 | STIM003- | Nucleic acid | GAAATTGTGTTGACGCAGTCTCCAGGGAC |
| | Light | sequence of V_{L} of | CCTGTCTTTGTCTCCAGGGGAAAGAGCCA |
| | chain | STIM003 | CCCTCTCCTGCAGGGCCAGTCAGAGTGTT |
| | variable | | AGCAGAAGCTACTTAGCCTGGTACCAGCA |
| | region | | GAAACGTGGCCAGGCTCCCAGGCTCCTCA |
| | | | TCTATGGTGCATCCAGCAGGGCCACTGGC |
| | | | ATCCCAGACAGGTTCAGTGGCGATGGGTC |
| | | | TGGGACAGACTTCACTCTCTCCATCAGCA |
| | | | GACTGGAGCCTGAAGATTTTGCAGTGTAT |
| | | | TACTGTCACCAGTATGATATGTCACCATTC |
| | | | ACTTTCGGCCCTGGGACCAAAGTGGATAT |
| | | | CAAA |
| 417 | STIM003 | Amino acid | EIVLTQSPGTLSLSPGERATLSCRASQSVSRS |
| | (KY1044)- | sequence of | YLAWYQQKRGQAPRLLIYGASSRATGIPDR |
| | full | STIM003 light chain | FSGDGSGTDFTLSISRLEPEDFAVYYCHQYD |
| | light | | MSPFTFGPGTKVDIKRTVAAPSVFIFPPSDEQ |
| | chain | | LKSGTASVVCLLNNFYPREAKVQWKVDNA |
| | sequence | | LQSGNSQESVTEQDSKDSTYSLSSTLTLSKA |
| | | | DYEKHKVYACEVTHQGLSSPVTKSFNRGEC |
| 418 | STIM003- | Nucleic acid | GAAATTGTGTTGACGCAGTCTCCAGGGAC |
| | full | sequence of | CCTGTCTTTGTCTCCAGGGGAAAGAGCCA |
| | light | STIM003 light chain | CCCTCTCCTGCAGGGCCAGTCAGAGTGTT |
| | chain | | AGCAGAAGCTACTTAGCCTGGTACCAGCA |
| | sequence | | GAAACGTGGCCAGGCTCCCAGGCTCCTCA |
| | | | TCTATGGTGCATCCAGCAGGGCCACTGGC |
| | | | ATCCCAGACAGGTTCAGTGGCGATGGGTC |
| | | | TGGGACAGACTTCACTCTCTCCATCAGCA |
| | | | GACTGGAGCCTGAAGATTTTGCAGTGTAT |
| | | | TACTGTCACCAGTATGATATGTCACCATTC |
| | | | ACTTTCGGCCCTGGGACCAAAGTGGATAT |
| | | | CAAAcgtacggtggccgctccctccgtgt |
| | | | tcatcttcccaccttccgacgagcagctg |
| | | | aagtccggcaccgcttctgtcgtgtgcct |
| | | | gctgaacaacttctacccccgcgaggcca |
| | | | caggtgcagtggaaggtggacaacgccct |
| | | | ggcagtccggcaactcccaggaatccgtg |
| | | | accgagcaggactcaaggacagcacctac |
| | | | tccctgtcctccaccctgaccctgtccaa |
| | | | gccgactacgagaagcacaaggtgtacgc |
| | | | ctgcgaagtgacccaccagggcctgtcta |
| | | | gccccgtgaccaagtctttcaaccggggc |
| | | | gagtgt |
| 419 | STIM004- | Amino acid | GLTFDDYG |
| | CDRH1 | sequence of CDRH1 | |
| | | of STIM004 using | |
| | | IMGT | |
| 420 | STIM004- | Amino acid | INWNGDNT |
| | CDRH2 | sequence of CDRH2 | |
| | | of STIM004 using | |
| | | IMGT | |
| 421 | STIM004- | Amino acid | ARDYYGSGSYYNVPFDY |
| | CDRH3 | sequence of CDRH3 | |
| | | of STIM004 using | |
| | | IMGT | |
| 422 | STIM004- | Amino acid | EVQLVESGGGVVRPGGSLRLSCAASGLTFD |
| | Heavy | sequence of V_{H} of | DYGMSWVRQVPGKGLEWVSGINWNGDNT |
| | chain | STIM004 | DYADSVKGRFTISRDNAKNSLYLQMNSLRA |
| | variable | | EDTALYYCARDYYGSGSYYNVPFDYWGQG |
| | region | | TLVTVSS |
| 423 | STIM004- | Nucleic acid | GAGGTGCAGCTGGTGGAGTCTGGGGGAGG |
| | Heavy | sequence of V_{H} of | TGTGGTACGGCCTGGGGGGTCCCTGAGAC |
| | chain | STIM004 | TCTCCTGTGCAGCCTCTGGACTCACCTTTG |
| | variable | | ATGATTATGGCATGAGCTGGGTCCGCCAA |
| | region | | GTTCCAGGGAAGGGGCTGGAGTGGGTCTC |
| | | | TGGTATTAATTGGAATGGTGATAACACAG |
| | | | ATTATGCAGACTCTGTGAAGGGCCGATTC |
| | | | ACCATCTCCAGAGACAACGCCAAGAACTC |
| | | | CCTGTATCTGCAAATGAACAGTCTGAGAG |
| | | | CCGAGGACACGGCCTTGTATTACTGTGCG |
| | | | AGGGATTACTATGGTTCGGGGAGTTATTA |
| | | | TAACGTTCCTTTTGACTACTGGGGCCAGG |
| | | | GAACCCTGGTCACCGTCTCCTCA |
| 424 | STIM004- | Amino acid | EVQLVESGGGVVRPGGSLRLSCAASGLTFD |
| | full | sequence of | DYGMSWVRQVPGKGLEWVSGINWNGDNT |
| | heavy | STIM004 heavy | DYADSVKGRFTISRDNAKNSLYLQMNSLRA |
| | chain | chain | EDTALYYCARDYYGSGSYYNVPFDYWGQG |
| | sequence | | TLVTVSSASTKGPSVFPLAPSSKSTSGGTAAL |
| | | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 425 | STIM004- | Nucleic acid | GAGGTGCAGCTGGTGGAGTCTGGGGGAGG |
| | full | sequence of | TGTGGTACGGCCTGGGGGGTCCCTGAGAC |
| | heavy | STIM004 heavy | TCTCCTGTGCAGCCTCTGGACTCACCTTTG |
| | chain | chain | ATGATTATGGCATGAGCTGGGTCCGCCAA |
| | sequence | | GTTCCAGGGAAGGGGCTGGAGTGGGTCTC |
| | | | TGGTATTAATTGGAATGGTGATAACACAG |
| | | | ATTATGCAGACTCTGTGAAGGGCCGATTC |
| | | | ACCATCTCCAGAGACAACGCCAAGAACTC |
| | | | CCTGTATCTGCAAATGAACAGTCTGAGAG |
| | | | CCGAGGACACGGCCTTGTATTACTGTGCG |
| | | | AGGGATTACTATGGTTCGGGGAGTTATTA |
| | | | TAACGTTCCTTTTGACTACTGGGGCCAGG |
| | | | GAACCCTGGTCACCGTCTCCTCAGCCAGC |
| | | | ACCAAGGGCCCCTCTGTGTTCCCTCTGGCC |
| | | | CCTTCCAGCAAGTCCACCTCTGGCGGAAC |
| | | | AGCCGCTCTGGGCTGCCTCGTGAAGGACT |
| | | | ACTTCCCCGAGCCTGTGACCGTGTCCTGG |
| | | | AACTCTGGCGCTCTGACCAGCGGAGTGCA |
| | | | CACCTTCCCTGCTGTGCTGCAGTCCTCCGG |
| | | | CCTGTACTCCCTGTCCTCCGTCGTGACCGT |
| | | | GCCTTCCAGCTCTCTGGGCACCCAGACCT |
| | | | ACATCTGCAACGTGAACCACAAGCCCTCC |
| | | | AACACCAAGGTGGACAAGAAGGTGGAAC |
| | | | CCAAGTCCTGCGACAAGACCCACACCTGT |
| | | | CCCCCTTGTCCTGCCCCTGAACTGCTGGGC |
| | | | GGACCTTCCGTGTTCCTGTTCCCCCCAAAG |
| | | | CCCAAGGACACCCTGATGATCTCCCGGAC |
| | | | CCCCGAAGTGACCTGCGTGGTGGTGGATG |
| | | | TGTCCCACGAGGACCCTGAAGTGAAGTTC |
| | | | AATTGGTACGTGGACGGCGTGGAAGTGCA |
| | | | CAACGCCAAGACCAAGCCTAGAGAGGAA |
| | | | CAGTACAACTCCACCTACCGGGTGGTGTC |
| | | | CGTGCTGACCGTGCTGCACCAGGATTGGC |
| | | | TGAACGGCAAAGAGTACAAGTGCAAGGT |
| | | | GTCCAACAAGGCCCTGCCTGCCCCCATCG |
| | | | AAAAGACCATCTCCAAGGCCAAGGGCCAG |
| | | | CCCCGGGAACCCCAGGTGTACACACTGCC |
| | | | CCCTAGCAGGGACGAGCTGACCAAGAACC |
| | | | AGGTGTCCCTGACCTGTCTCGTGAAAGGC |
| | | | TTCTACCCCTCCGATATCGCCGTGGAATGG |
| | | | GAGTCCAACGGCCAGCCTGAGAACAACTA |
| | | | CAAGACCACCCCCCCTGTGCTGGACTCCG |
| | | | ACGGCTCATTCTTCCTGTACAGCAAGCTG |
| | | | ACAGTGGACAAGTCCCGGTGGCAGCAGGG |
| | | | CAACGTGTTCTCCTGCTCCGTGATGCACGA |
| | | | GGCCCTGCACAACCACTACACCCAGAAGT |
| | | | CCCTGTCCCTGAGCCCCGGCAAGTGATGA |
| 426 | STIM004- | Amino acid | QSVSSSY |
| | CDRL1 | sequence of CDRL1 | |
| | | of STIM004 using | |
| | | IMGT | |
| 427 | STIM004- | Amino acid | GAS |
| | CDRL2 | sequence of CDRL2 | |
| | | of STIM004 using | |
| | | IMGT | |
| 428 | STIM004- | Amino acid | QQYGSSPF |
| | CDRL3 | sequence of CDRL3 | |
| | | of STIM004 using | |
| | | IMGT | |
| 429 | STIM004- | Amino acid | EIVLTQSPGTLSLSPGERATLSCRASQSVSSS |
| | Corrected | sequence of | YLAWYQQKPGQAPRLLIYGASSRATGIPDRF |
| | light | corrected V_{L} of | SGSGSGTDFTLTIRRLEPEDFAVYYCQQYGS |
| | chain | STIM004 | SPFFGPGTKVDIK |
| | variable | | |
| | region | | |
| 430 | STIM004- | Nucleic acid | GAAATTGTGTTGACGCAGTCTCCAGGCAC |
| | Corrected | sequence of | CCTGTCTTTGTCTCCAGGGGAAAGAGCCA |
| | light | corrected V_{L} of | CCCTCTCCTGCAGGGCCAGTCAGAGTGTT |
| | chain | STIM004 | AGCAGCAGCTACTTAGCCTGGTACCAGCA |
| | variable | | GAAACCTGGCCAGGCTCCCAGGCTCCTCA |
| | region | | TATATGGTGCATCCAGCAGGGCCACTGGC |
| | | | ATCCCAGACAGGTTCAGTGGCAGTGGGTC |
| | | | TGGGACAGACTTCACTCTCACCATCAGAA |
| | | | GACTGGAGCCTGAAGATTTTGCAGTGTAT |
| | | | TACTGTCAGCAGTATGGTAGTTCACCATTC |
| | | | TTCGGCCCTGGGACCAAAGTGGATATCAA |
| | | | A |
| 431 | STIM004- | Nucleic acid | GAAATTGTGTTGACGCAGTCTCCAGGCAC |
| | Light | sequence of V_{L} of | CCTGTCTTTGTCTCCAGGGGAAAGAGCCA |
| | chain | STIM004 | CCCTCTCCTGCAGGGCCAGTCAGAGTGTT |
| | variable | | AGCAGCAGCTACTTAGCCTGGTACCAGCA |
| | region | | GAAACCTGGCCAGGCTCCCAGGCTCCTCA |
| | | | TATATGGTGCATCCAGCAGGGCCACTGGC |
| | | | ATCCCAGACAGGTTCAGTGGCAGTGGGTC |
| | | | TGGGACAGACTTCACTCTCACCATCAGAA |
| | | | GACTGGAGCCTGAAGATTTTGCAGTGTAT |
| | | | TACTGTCAGCAGTATGGTAGTTCACCATTC |
| | | | ACTTCGGCCCTGGGACCAAAGTGGATATC |
| | | | AAA |
| 432 | STIM004- | Amino acid | EIVLTQSPGTLSLSPGERATLSCRASQSVSSS |
| | full | sequence of | YLAWYQQKPGQAPRLLIYGASSRATGIPDRF |
| | corrected | STIM004 light chain | SGSGSGTDFTLTIRRLEPEDFAVYYCQQYGS |
| | light | | SPFFGPGTKVDIKRTVAAPSVFIFPPSDEQLK |
| | chain | | SGTASVVCLLNNFYPREAKVQWKVDNALQ |
| | sequence | | SGNSQESVTEQDSKDSTYSLSSTLTLSKADY |
| | | | EKHKVYACEVTHQGLSSPVTKSFNRGEC |
| 433 | STIM004- | Nucleic acid | GAAATTGTGTTGACGCAGTCTCCAGGCAC |
| | full | sequence of | CCTGTCTTTGTCTCCAGGGGAAAGAGCCA |
| | corrected | corrected STIM004 | CCCTCTCCTGCAGGGCCAGTCAGAGTGTT |
| | light | light chain | AGCAGCAGCTACTTAGCCTGGTACCAGCA |
| | chain | | GAAACCTGGCCAGGCTCCCAGGCTCCTCA |
| | sequence | | TATATGGTGCATCCAGCAGGGCCACTGGC |
| | | | ATCCCAGACAGGTTCAGTGGCAGTGGGTC |
| | | | TGGGACAGACTTCACTCTCACCATCAGAA |
| | | | GACTGGAGCCTGAAGATTTTGCAGTGTAT |
| | | | TACTGTCAGCAGTATGGTAGTTCACCATTC |
| | | | TTCGGCCCTGGGACCAAAGTGGATATCAA |
| | | | Acgtacggtggccgctccctccgtgttcatc |
| | | | ttcccaccttccgacgagcagctgaagtcc |
| | | | ggcaccgcttctgtcgtgtgcctgctgaaca |
| | | | acttctacccccgcgaggccaaggtgcagtg |
| | | | gaaggtggacaacgccctgcagtccggcaac |
| | | | tcccaggaatccgtgaccgagcaggactcca |
| | | | aggacagcacctactccctgtcctccaccct |
| | | | gaccctgtccaaggccgactacgagaagcac |
| | | | aaggtgtacgcctgcgaagtgacccaccagg |
| | | | gcctgtctagccccgtgaccaagtctttcaa |
| | | | ccggggcgagtgt |
| 434 | STIM004- | Nucleic acid | GAAATTGTGTTGACGCAGTCTCCAGGCAC |
| | full | sequence of | CCTGTCTTTGTCTCCAGGGGAAAGAGCCA |
| | light | STIM004 light chain | CCCTCTCCTGCAGGGCCAGTCAGAGTGTT |
| | chain | | AGCAGCAGCTACTTAGCCTGGTACCAGCA |
| | sequence | | GAAACCTGGCCAGGCTCCCAGGCTCCTCA |
| | | | TATATGGTGCATCCAGCAGGGCCACTGGC |
| | | | ATCCCAGACAGGTTCAGTGGCAGTGGGTC |
| | | | TGGGACAGACTTCACTCTCACCATCAGAA |
| | | | GACTGGAGCCTGAAGATTTTGCAGTGTAT |
| | | | TACTGTCAGCAGTATGGTAGTTCACCATTC |
| | | | ACTTCGGCCCTGGGACCAAAGTGGATATC |
| | | | AAAcgtacggtggccgctccctccgtgttca |
| | | | tcttcccaccttccgacgagcagctgaagtc |
| | | | cggcaccgcttctgtcgtgtgcctgctgaac |
| | | | aacttctacccccgcgaggccaaggtgcagt |
| | | | ggaaggtggacaacgccctgcagtccggcaa |
| | | | ctcccaggaatccgtgaccgagcaggactcc |
| | | | aaggacagcacctactccctgtcctccaccc |
| | | | tgaccctgtccaaggccgactacgagaagca |
| | | | caaggtgtacgcctgcgaagtgacccaccag |
| | | | ggcctgtctagccccgtgaccaagtctttca |
| | | | accggggcgagtgt |
| 435 | STIM005- | Amino acid | GYTFNSYG |
| | CDRH1 | sequence of CDRH1 | |
| | | of STIM005 using | |
| | | IMGT | |
| 436 | STIM005- | Amino acid | ISVHNGNT |
| | CDRH2 | sequence of CDRH2 | |
| | | of STIM005 using | |
| | | IMGT | |
| 437 | STIM005- | Amino acid | ARAGYDILTDFSDAFDI |
| | CDRH3 | sequence of CDRH3 | |
| | | of STIM005 using | |
| | | IMGT | |
| 438 | STIM005- | Amino acid | QVQLVQSGAEVKKPGASVKVSCKASGYTF |
| | Heavy | sequence of V_{H} of | NSYGIIWVRQAPGQGLEWMGWISVHNGNT |
| | chain | STIM005 | NCAQKLQGRVTMTTDTSTSTAYMELRSLRT |
| | variable | | DDTAVYYCARAGYDILTDFSDAFDIWGHGT |
| | region | | MVTVSS |
| 439 | STIM005- | Nucleic acid | CAGGTTCAGTTGGTGCAGTCTGGAGCTGA |
| | Heavy | sequence of V_{H} of | GGTGAAGAAGCCTGGGGCCTCAGTGAAGG |
| | chain | STIM005 | TCTCCTGCAAGGCTTCTGGTTACACCTTTA |
| | variable | | ATAGTTATGGTATCATCTGGGTGCGACAG |
| | region | | GCCCCTGGACAAGGGCTTGAGTGGATGGG |
| | | | ATGGATCAGCGTTCACAATGGTAACACAA |
| | | | ACTGTGCACAGAAGCTCCAGGGTAGAGTC |
| | | | ACCATGACCACAGACACATCCACGAGCAC |
| | | | AGCCTACATGGAGCTGAGGAGCCTGAGAA |
| | | | CTGACGACACGGCCGTGTATTACTGTGCG |
| | | | AGAGCGGGTTACGATATTTTGACTGATTTT |
| | | | TCCGATGCTTTTGATATCTGGGGCCACGG |
| | | | GACAATGGTCACCGTCTCTTCA |
| 440 | STIM005- | Amino acid | QVQLVQSGAEVKKPGASVKVSCKASGYTF |
| | full | sequence of | NSYGIIWVRQAPGQGLEWMGWISVHNGNT |
| | heavy | STIM005 heavy | NCAQKLQGRVTMTTDTSTSTAYMELRSLRT |
| | chain | chain | DDTAVYYCARAGYDILTDFSDAFDIWGHGT |
| | sequence | | MVTVSSASTKGPSVFPLAPSSKSTSGGTAAL |
| | | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 441 | STIM005- | Nucleic acid | CAGGTTCAGTTGGTGCAGTCTGGAGCTGA |
| | full | sequence of | GGTGAAGAAGCCTGGGGCCTCAGTGAAGG |
| | heavy | STIM005 heavy | TCTCCTGCAAGGCTTCTGGTTACACCTTTA |
| | chain | chain | ATAGTTATGGTATCATCTGGGTGCGACAG |
| | sequence | | GCCCCTGGACAAGGGCTTGAGTGGATGGG |
| | | | ATGGATCAGCGTTCACAATGGTAACACAA |
| | | | ACTGTGCACAGAAGCTCCAGGGTAGAGTC |
| | | | ACCATGACCACAGACACATCCACGAGCAC |
| | | | AGCCTACATGGAGCTGAGGAGCCTGAGAA |
| | | | CTGACGACACGGCCGTGTATTACTGTGCG |
| | | | AGAGCGGGTTACGATATTTTGACTGATTTT |
| | | | TCCGATGCTTTTGATATCTGGGGCCACGG |
| | | | GACAATGGTCACCGTCTCTTCA |
| | | | GCCAGCACCAAGGGCCCCTCTGTGTTCCC |
| | | | TCTGGCCCCTTCCAGCAAGTCCACCTCTGG |
| | | | CGGAACAGCCGCTCTGGGCTGCCTCGTGA |
| | | | AGGACTACTTCCCCGAGCCTGTGACCGTG |
| | | | TCCTGGAACTCTGGCGCTCTGACCAGCGG |
| | | | AGTGCACACCTTCCCTGCTGTGCTGCAGTC |
| | | | CTCCGGCCTGTACTCCCTGTCCTCCGTCGT |
| | | | GACCGTGCCTTCCAGCTCTCTGGGCACCC |
| | | | AGACCTACATCTGCAACGTGAACCACAAG |
| | | | CCCTCCAACACCAAGGTGGACAAGAAGGT |
| | | | GGAACCCAAGTCCTGCGACAAGACCCACA |
| | | | CCTGTCCCCCTTGTCCTGCCCCTGAACTGC |
| | | | TGGGCGGACCTTCCGTGTTCCTGTTCCCCC |
| | | | CAAAGCCCAAGGACACCCTGATGATCTCC |
| | | | CGGACCCCCGAAGTGACCTGCGTGGTGGT |
| | | | GGATGTGTCCCACGAGGACCCTGAAGTGA |
| | | | AGTTCAATTGGTACGTGGACGGCGTGGAA |
| | | | GTGCACAACGCCAAGACCAAGCCTAGAGA |
| | | | GGAACAGTACAACTCCACCTACCGGGTGG |
| | | | TGTCCGTGCTGACCGTGCTGCACCAGGAT |
| | | | TGGCTGAACGGCAAAGAGTACAAGTGCAA |
| | | | GGTGTCCAACAAGGCCCTGCCTGCCCCCA |
| | | | TCGAAAAGACCATCTCCAAGGCCAAGGGC |
| | | | CAGCCCCGGGAACCCCAGGTGTACACACT |
| | | | GCCCCCTAGCAGGGACGAGCTGACCAAGA |
| | | | ACCAGGTGTCCCTGACCTGTCTCGTGAAA |
| | | | GGCTTCTACCCCTCCGATATCGCCGTGGA |
| | | | ATGGGAGTCCAACGGCCAGCCTGAGAACA |
| | | | ACTACAAGACCACCCCCCCTGTGCTGGAC |
| | | | TCCGACGGCTCATTCTTCCTGTACAGCAAG |
| | | | CTGACAGTGGACAAGTCCCGGTGGCAGCA |
| | | | GGGCAACGTGTTCTCCTGCTCCGTGATGC |
| | | | ACGAGGCCCTGCACAACCACTACACCCAG |
| | | | AAGTCCCTGTCCCTGAGCCCCGGCAAGTG |
| | | | ATGA |
| 442 | STIM005- | Amino acid | QNINNF |
| | CDRL1 | sequence of CDRL1 | |
| | | of STIM005 using | |
| | | IMGT | |
| 443 | STIM005- | Amino acid | AAS |
| | CDRL2 | sequence of CDRL2 | |
| | | of STIM005 using | |
| | | IMGT | |
| 444 | STIM005- | Amino acid | QQSYGIPW |
| | CDRL3 | sequence of CDRL3 | |
| | | of STIM005 using | |
| | | IMGT | |
| 445 | STIM005- | Amino acid | DIQMTQSPSSLSASVGDRVTITCRASQNINNF |
| | Light | sequence of V_{L} of | LNWYQQKEGKGPKLLIYAASSLQRGIPSTFS |
| | chain | STIM005 | GSGSGTDFTLTISSLQPEDFATYICQQSYGIP |
| | variable | | WVGQGTKVEIK |
| | region | | |
| 446 | STIM005- | Nucleic acid | GACATCCAGATGACCCAGTCTCCATCCTC |
| | Light | sequence of V_{L} of | CCTGTCTGCATCTGTAGGAGACAGAGTCA |
| | chain | STIM005 | CCATCACTTGCCGGGCAAGTCAGAACATT |
| | variable | | AATAACTTTTTAAATTGGTATCAGCAGAA |
| | region | | AGAAGGGAAAGGCCCTAAGCTCCTGATCT |
| | | | ATGCAGCATCCAGTTTGCAAAGAGGGATA |
| | | | CCATCAACGTTCAGTGGCAGTGGATCTGG |
| | | | GACAGACTTCACTCTCACCATCAGCAGTC |
| | | | TGCAACCTGAAGATTTTGCAACTTACATCT |
| | | | GTCAACAGAGCTACGGTATCCCGTGGGTC |
| | | | GGCCAAGGGACCAAGGTGGAAATCAAA |
| 447 | STIM005- | Amino acid | DIQMTQSPSSLSASVGDRVTITCRASQNINNF |
| | full | sequence of | LNWYQQKEGKGPKLLIYAASSLQRGIPSTFS |
| | light | STIM005 light chain | GSGSGTDFTLTISSLQPEDFATYICQQSYGIP |
| | chain | | WVGQGTKVEIK |
| | sequence | | RTVAAPSVFIFPPSDEQLKSGTASVVCLLNN |
| | | | FYPREAKVQWKVDNALQSGNSQESVTEQD |
| | | | SKDSTYSLSSTLTLSKADYEKHKVYACEVT |
| | | | HQGLSSPVTKSFNRGEC |
| 448 | STIM005- | Nucleic acid | GACATCCAGATGACCCAGTCTCCATCCTC |
| | full | sequence of | CCTGTCTGCATCTGTAGGAGACAGAGTCA |
| | light | STIM005 light chain | CCATCACTTGCCGGGCAAGTCAGAACATT |
| | chain | | AATAACTTTTTAAATTGGTATCAGCAGAA |
| | sequence | | AGAAGGGAAAGGCCCTAAGCTCCTGATCT |
| | | | ATGCAGCATCCAGTTTGCAAAGAGGGATA |
| | | | CCATCAACGTTCAGTGGCAGTGGATCTGG |
| | | | GACAGACTTCACTCTCACCATCAGCAGTC |
| | | | TGCAACCTGAAGATTTTGCAACTTACATCT |
| | | | GTCAACAGAGCTACGGTATCCCGTGGGTC |
| | | | GGCCAAGGGACCAAGGTGGAAATCAAAcgt |
| | | | acggtggccgctccctccgtgttcatcttc |
| | | | ccaccttccgacgagcagctgaagtccggc |
| | | | accgcttctgtcgtgtgcctgctgaacaac |
| | | | ttctacccccgcgaggccaaggtgcagtgg |
| | | | aaggtggacaacgccctgcagtccggcaac |
| | | | tcccaggaatccgtgaccgagcaggactcc |
| | | | aaggacagcacctactccctgtcctccacc |
| | | | ctgaccctgtccaaggccgactacgagaag |
| | | | cacaaggtgtacgcctgcgaagtgacccac |
| | | | cagggcctgtctagccccgtgaccaagtct |
| | | | ttcaaccggggcgagtgt |
| 449 | STIM006- | Amino acid | GFTFSDYF |
| | CDRH1 | sequence of CDRH1 | |
| | | of STIM006 using | |
| | | IMGT | |
| 450 | STIM006- | Amino acid | ISSSGSTI |
| | CDRH2 | sequence of CDRH2 | |
| | | of STIM006 using | |
| | | IMGT | |
| 451 | STIM006- | Amino acid | ARDHYDGSGIYPLYYYYGLDV |
| | CDRH3 | sequence of CDRH3 | |
| | | of STIM006 using | |
| | | IMGT | |
| 452 | STIM006- | Amino acid | QVQLVESGGGLVKPGGSLRLSCAASGFTFS |
| | Heavy | sequence of V_{H} of | DYFMSWIRQAPGKGLEWISYISSSGSTIYYA |
| | chain | STIM006 | DSVRGRFTISRDNAKYSLYLQMNSLRSEDT |
| | variable | | AVYYCARDHYDGSGIYPLYYYYGLDVWGQ |
| | region | | GTTVTVSS |
| 453 | STIM006- | Nucleic acid | CAGGTGCAGCTGGTGGAGTCTGGGGGAGG |
| | Heavy | sequence of V_{H} of | CTTGGTCAAGCCTGGAGGGTCCCTGAGAC |
| | chain | STIM006 | TCTCCTGTGCAGCCTCTGGATTCACCTTCA |
| | variable | | GTGACTACTTCATGAGCTGGATCCGCCAG |
| | region | | GCGCCAGGGAAGGGGCTGGAGTGGATTTC |
| | | | ATACATTAGTTCTAGTGGTAGTACCATATA |
| | | | CTACGCAGACTCTGTGAGGGGCCGATTCA |
| | | | CCATCTCCAGGGACAACGCCAAGTACTCA |
| | | | CTGTATCTGCAAATGAACAGCCTGAGATC |
| | | | CGAGGACACGGCCGTGTATTACTGTGCGA |
| | | | GAGATCACTACGATGGTTCGGGGATTTAT |
| | | | CCCCTCTACTACTATTACGGTTTGGACGTC |
| | | | TGGGGCCAGGGGACCACGGTCACCGTCTC |
| | | | CTCA |
| 454 | STIM006- | Amino acid | QVQLVESGGGLVKPGGSLRLSCAASGFTFS |
| | full | sequence of | DYFMSWIRQAPGKGLEWISYISSSGSTIYYA |
| | heavy | STIM006 heavy | DSVRGRFTISRDNAKYSLYLQMNSLRSEDT |
| | chain | chain | AVYYCARDHYDGSGIYPLYYYYGLDVWGQ |
| | sequence | | GTTVTVSSASTKGPSVFPLAPSSKSTSGGTA |
| | | | ALGCLVKDYFPEPVTVSWNSGALTSGVHTF |
| | | | PAVLQSSGLYSLSSVVTVPSSSLGTQTYICNV |
| | | | NHKPSNTKVDKKVEPKSCDKTHTCPPCPAP |
| | | | ELLGGPSVFLFPPKPKDTLMISRTPEVTCVV |
| | | | VDVSHEDPEVKFNWYVDGVEVHNAKTKPR |
| | | | EEQYNSTYRVVSVLTVLHQDWLNGKEYKC |
| | | | KVSNKALPAPIEKTISKAKGQPREPQVYTLP |
| | | | PSRDELTKNQVSLTCLVKGFYPSDIAVEWES |
| | | | NGQPENNYKTTPPVLDSDGSFFLYSKLTVD |
| | | | KSRWQQGNVFSCSVMHEALHNHYTQKSLS |
| | | | LSPGK |
| 455 | STIM006- | Nucleic acid | CAGGTGCAGCTGGTGGAGTCTGGGGGAGG |
| | full | sequence of | CTTGGTCAAGCCTGGAGGGTCCCTGAGAC |
| | heavy | STIM006 heavy | TCTCCTGTGCAGCCTCTGGATTCACCTTCA |
| | chain | chain | GTGACTACTTCATGAGCTGGATCCGCCAG |
| | sequence | | GCGCCAGGGAAGGGGCTGGAGTGGATTTC |
| | | | ATACATTAGTTCTAGTGGTAGTACCATATA |
| | | | CTACGCAGACTCTGTGAGGGGCCGATTCA |
| | | | CCATCTCCAGGGACAACGCCAAGTACTCA |
| | | | CTGTATCTGCAAATGAACAGCCTGAGATC |
| | | | CGAGGACACGGCCGTGTATTACTGTGCGA |
| | | | GAGATCACTACGATGGTTCGGGGATTTAT |
| | | | CCCCTCTACTACTATTACGGTTTGGACGTC |
| | | | TGGGGCCAGGGGACCACGGTCACCGTCTC |
| | | | CTCAGCCAGCACCAAGGGCCCCTCTGTGT |
| | | | TCCCTCTGGCCCCTTCCAGCAAGTCCACCT |
| | | | CTGGCGGAACAGCCGCTCTGGGCTGCCTC |
| | | | GTGAAGGACTACTTCCCCGAGCCTGTGAC |
| | | | CGTGTCCTGGAACTCTGGCGCTCTGACCA |
| | | | GCGGAGTGCACACCTTCCCTGCTGTGCTG |
| | | | CAGTCCTCCGGCCTGTACTCCCTGTCCTCC |
| | | | GTCGTGACCGTGCCTTCCAGCTCTCTGGGC |
| | | | ACCCAGACCTACATCTGCAACGTGAACCA |
| | | | CAAGCCCTCCAACACCAAGGTGGACAAGA |
| | | | AGGTGGAACCCAAGTCCTGCGACAAGACC |
| | | | CACACCTGTCCCCCTTGTCCTGCCCCTGAA |
| | | | CTGCTGGGCGGACCTTCCGTGTTCCTGTTC |
| | | | CCCCCAAAGCCCAAGGACACCCTGATGAT |
| | | | CTCCCGGACCCCCGAAGTGACCTGCGTGG |
| | | | TGGTGGATGTGTCCCACGAGGACCCTGAA |
| | | | GTGAAGTTCAATTGGTACGTGGACGGCGT |
| | | | GGAAGTGCACAACGCCAAGACCAAGCCTA |
| | | | GAGAGGAACAGTACAACTCCACCTACCGG |
| | | | GTGGTGTCCGTGCTGACCGTGCTGCACCA |
| | | | GGATTGGCTGAACGGCAAAGAGTACAAGT |
| | | | GCAAGGTGTCCAACAAGGCCCTGCCTGCC |
| | | | CCCATCGAAAAGACCATCTCCAAGGCCAA |
| | | | GGGCCAGCCCCGGGAACCCCAGGTGTACA |
| | | | CACTGCCCCCTAGCAGGGACGAGCTGACC |
| | | | AAGAACCAGGTGTCCCTGACCTGTCTCGT |
| | | | GAAAGGCTTCTACCCCTCCGATATCGCCG |
| | | | TGGAATGGGAGTCCAACGGCCAGCCTGAG |
| | | | AACAACTACAAGACCACCCCCCCTGTGCT |
| | | | GGACTCCGACGGCTCATTCTTCCTGTACAG |
| | | | CAAGCTGACAGTGGACAAGTCCCGGTGGC |
| | | | AGCAGGGCAACGTGTTCTCCTGCTCCGTG |
| | | | ATGCACGAGGCCCTGCACAACCACTACAC |
| | | | CCAGAAGTCCCTGTCCCTGAGCCCCGGCA |
| | | | AGTGATGA |
| 456 | STIM006- | Amino acid | QSLLHSNGYNY |
| | CDRL1 | sequence of CDRL1 | |
| | | of STIM006 using | |
| | | IMGT | |
| 457 | STIM006- | Amino acid | LGS |
| | CDRL2 | sequence of CDRL2 | |
| | | of STIM006 using | |
| | | IMGT | |
| 458 | STIM006- | Amino acid | MQALQTPRS |
| | CDRL3 | sequence of CDRL3 | |
| | | of STIM006 using | |
| | | IMGT | |
| 459 | STIM006- | Amino acid | IVMTQSPLSLPVTPGEPASISCRSSQSLLHSN |
| | Light | sequence of V_{L} of | GYNYLDYYLQKPGQSPQLLIYLGSYRASGV |
| | chain | STIM006 | PDRFSGSGSGTDFTLKISRVEAEDVGVYYC |
| | variable | | MQALQTPRSFGQGTTLEIK |
| | region | | |
| 460 | STIM006- | Nucleic acid | ATTGTGATGACTCAGTCTCCACTCTCCCTA |
| | Light | sequence of V_{L} of | CCCGTCACCCCTGGAGAGCCGGCCTCCAT |
| | chain | STIM006 | CTCCTGCAGGTCTAGTCAGAGCCTCCTGC |
| | variable | | ATAGTAATGGATACAACTATTTGGATTATT |
| | region | | ACCTGCAGAAGCCAGGGCAGTCTCCACAG |
| | | | CTCCTGATCTATTTGGGTTCTTATCGGGCC |
| | | | TCCGGGGTCCCTGACAGGTTCAGTGGCAG |
| | | | TGGATCAGGCACAGATTTTACACTGAAAA |
| | | | TCAGCAGAGTGGAGGCTGAGGATGTTGGG |
| | | | GTTTATTACTGCATGCAAGCTCTACAAACT |
| | | | CCTCGCAGTTTTGGCCAGGGGACCACGCT |
| | | | GGAGATCAAA |
| 461 | STIM006- | Amino acid | IVMTQSPLSLPVTPGEPASISCRSSQSLLHSN |
| | full | sequence of | GYNYLDYYLQKPGQSPQLLIYLGSYRASGV |
| | light | STIM006 light chain | PDRFSGSGSGTDFTLKISRVEAEDVGVYYC |
| | chain | | MQALQTPRSFGQGTTLEIKRTVAAPSVFIFPP |
| | sequence | | SDEQLKSGTASVVCLLNNFYPREAKVQWK |
| | | | VDNALQSGNSQESVTEQDSKDSTYSLSSTLT |
| | | | LSKADYEKHKVYACEVTHQGLSSPVTKSFN |
| | | | RGEC |
| 462 | STIM006- | Nucleic acid | ATTGTGATGACTCAGTCTCCACTCTCCCTA |
| | full | sequence of | CCCGTCACCCCTGGAGAGCCGGCCTCCAT |
| | light | STIM006 light chain | CTCCTGCAGGTCTAGTCAGAGCCTCCTGC |
| | chain | | ATAGTAATGGATACAACTATTTGGATTATT |
| | sequence | | ACCTGCAGAAGCCAGGGCAGTCTCCACAG |
| | | | CTCCTGATCTATTTGGGTTCTTATCGGGCC |
| | | | TCCGGGGTCCCTGACAGGTTCAGTGGCAG |
| | | | TGGATCAGGCACAGATTTTACACTGAAAA |
| | | | TCAGCAGAGTGGAGGCTGAGGATGTTGGG |
| | | | GTTTATTACTGCATGCAAGCTCTACAAACT |
| | | | CCTCGCAGTTTTGGCCAGGGGACCACGCT |
| | | | GGAGATCAAAcgtacggtggccgctccctcc |
| | | | gtgttcatcttcccaccttccgacgagcagct |
| | | | gaagtccggcaccgcttctgtcgtgtgcctgc |
| | | | tgaacaacttctacccccgcgaggccaaggtg |
| | | | gcagtggaaggtggacaacgccctgcagtccg |
| | | | gcaactcccaggaatccgtgaccagcaggact |
| | | | ccaaggacagcacctactccctgtcctccacc |
| | | | ctgaccctgtccaaggccgactacgagaagca |
| | | | caaggtgtacgcctgcgaagtgacccaccagg |
| | | | gcctgtctagccccgtgaccaagtctttcaac |
| | | | cggggcgagtgt |
| 463 | STIM007- | Amino acid | GFSLSTTGVG |
| | CDRH1 | sequence of CDRH1 | |
| | | of STIM007 using | |
| | | IMGT | |
| 464 | STIM007- | Amino acid | IYWDDDK |
| | CDRH2 | sequence of CDRH2 | |
| | | of STIM007 using | |
| | | IMGT | |
| 465 | STIM007- | Amino acid | THGYGSASYYHYGMDV |
| | CDRH3 | sequence of CDRH3 | |
| | | of STIM007 using | |
| | | IMGT | |
| 466 | STIM007- | Amino acid | QITLKESGPTLVKPTQTLTLTCTFSGFSLSTT |
| | Heavy | sequence of V_{H} of | GVGVGWIRQPPGKALEWLAVIYWDDDKRY |
| | chain | STIM007 | SPSLKSRLTITKDTSKNQVVLTMTNMDPVD |
| | variable | | TATYFCTHGYGSASYYHYGMDVWGQGTTV |
| | region | | TVSS |
| 467 | STIM007- | Nucleic acid | CAGATCACCTTGAAGGAGTCTGGTCCTAC |
| | Heavy | sequence of V_{H} of | GCTGGTGAAACCCACACAGACCCTCACGC |
| | chain | STIM007 | TGACCTGCACCTTCTCTGGGTTCTCACTCA |
| | variable | | GCACTACTGGAGTGGGTGTGGGCTGGATC |
| | region | | CGTCAGCCCCCAGGAAAGGCCCTGGAGTG |
| | | | GCTTGCAGTCATTTATTGGGATGATGATA |
| | | | AGCGCTACAGCCCATCTCTGAAGAGCAGA |
| | | | CTCACCATCACCAAGGACACCTCCAAAAA |
| | | | CCAGGTGGTCCTTACAATGACCAACATGG |
| | | | ACCCTGTGGACACAGCCACATATTTCTGT |
| | | | ACACACGGATATGGTTCGGCGAGTTATTA |
| | | | CCACTACGGTATGGACGTCTGGGGCCAAG |
| | | | GGACCACGGTCACCGTCTCCTCA |
| 468 | STIM007- | Amino acid | QITLKESGPTLVKPTQTLTLTCTFSGFSLSTT |
| | full | sequence of | GVGVGWIRQPPGKALEWLAVIYWDDDKRY |
| | heavy | STIM007 heavy | SPSLKSRLTITKDTSKNQVVLTMTNMDPVD |
| | chain | chain | TATYFCTHGYGSASYYHYGMDVWGQGTTV |
| | sequence | | TVSSASTKGPSVFPLAPSSKSTSGGTAALGC |
| | | | LVKDYFPEPVTVSWNSGALTSGVHTFPAVL |
| | | | QSSGLYSLSSVVTVPSSSLGTQTYICNVNHK |
| | | | PSNTKVDKKVEPKSCDKTHTCPPCPAPELLG |
| | | | GPSVFLFPPKPKDTLMISRTPEVTCVVVDVS |
| | | | HEDPEVKFNWYVDGVEVHNAKTKPREEQY |
| | | | NSTYRVVSVLTVLHQDWLNGKEYKCKVSN |
| | | | KALPAPIEKTISKAKGQPREPQVYTLPPSRDE |
| | | | LTKNQVSLTCLVKGFYPSDIAVEWESNGQP |
| | | | ENNYKTTPPVLDSDGSFFLYSKLTVDKSRW |
| | | | QQGNVFSCSVMHEALHNHYTQKSLSLSPGK |
| 469 | STIM007- | Nucleic acid | CAGATCACCTTGAAGGAGTCTGGTCCTAC |
| | full | sequence of | GCTGGTGAAACCCACACAGACCCTCACGC |
| | heavy | STIM007 heavy | TGACCTGCACCTTCTCTGGGTTCTCACTCA |
| | chain | chain | GCACTACTGGAGTGGGTGTGGGCTGGATC |
| | sequence | | CGTCAGCCCCCAGGAAAGGCCCTGGAGTG |
| | | | GCTTGCAGTCATTTATTGGGATGATGATA |
| | | | AGCGCTACAGCCCATCTCTGAAGAGCAGA |
| | | | CTCACCATCACCAAGGACACCTCCAAAAA |
| | | | CCAGGTGGTCCTTACAATGACCAACATGG |
| | | | ACCCTGTGGACACAGCCACATATTTCTGT |
| | | | ACACACGGATATGGTTCGGCGAGTTATTA |
| | | | CCACTACGGTATGGACGTCTGGGGCCAAG |
| | | | GGACCACGGTCACCGTCTCCTCA |
| | | | GCCAGCACCAAGGGCCCCTCTGTGTTCCC |
| | | | TCTGGCCCCTTCCAGCAAGTCCACCTCTGG |
| | | | CGGAACAGCCGCTCTGGGCTGCCTCGTGA |
| | | | AGGACTACTTCCCCGAGCCTGTGACCGTG |
| | | | TCCTGGAACTCTGGCGCTCTGACCAGCGG |
| | | | AGTGCACACCTTCCCTGCTGTGCTGCAGTC |
| | | | CTCCGGCCTGTACTCCCTGTCCTCCGTCGT |
| | | | GACCGTGCCTTCCAGCTCTCTGGGCACCC |
| | | | AGACCTACATCTGCAACGTGAACCACAAG |
| | | | CCCTCCAACACCAAGGTGGACAAGAAGGT |
| | | | GGAACCCAAGTCCTGCGACAAGACCCACA |
| | | | CCTGTCCCCCTTGTCCTGCCCCTGAACTGC |
| | | | TGGGCGGACCTTCCGTGTTCCTGTTCCCCC |
| | | | CAAAGCCCAAGGACACCCTGATGATCTCC |
| | | | CGGACCCCCGAAGTGACCTGCGTGGTGGT |
| | | | GGATGTGTCCCACGAGGACCCTGAAGTGA |
| | | | AGTTCAATTGGTACGTGGACGGCGTGGAA |
| | | | GTGCACAACGCCAAGACCAAGCCTAGAGA |
| | | | GGAACAGTACAACTCCACCTACCGGGTGG |
| | | | TGTCCGTGCTGACCGTGCTGCACCAGGAT |
| | | | TGGCTGAACGGCAAAGAGTACAAGTGCAA |
| | | | GGTGTCCAACAAGGCCCTGCCTGCCCCCA |
| | | | TCGAAAAGACCATCTCCAAGGCCAAGGGC |
| | | | CAGCCCCGGGAACCCCAGGTGTACACACT |
| | | | GCCCCCTAGCAGGGACGAGCTGACCAAGA |
| | | | ACCAGGTGTCCCTGACCTGTCTCGTGAAA |
| | | | GGCTTCTACCCCTCCGATATCGCCGTGGA |
| | | | ATGGGAGTCCAACGGCCAGCCTGAGAACA |
| | | | ACTACAAGACCACCCCCCCTGTGCTGGAC |
| | | | TCCGACGGCTCATTCTTCCTGTACAGCAAG |
| | | | CTGACAGTGGACAAGTCCCGGTGGCAGCA |
| | | | GGGCAACGTGTTCTCCTGCTCCGTGATGC |
| | | | ACGAGGCCCTGCACAACCACTACACCCAG |
| | | | AAGTCCCTGTCCCTGAGCCCCGGCAAGTG |
| | | | ATGA |
| 470 | STIM007- | Amino acid | QSVTNY |
| | CDRL1 | sequence of CDRL1 | |
| | | of STIM007 using | |
| | | IMGT | |
| 471 | STIM007- | Amino acid | DAS |
| | CDRL2 | sequence of CDRL2 | |
| | | of STIM007 using | |
| | | IMGT | |
| 472 | STIM007- | Amino acid | QHRSNWPLT |
| | CDRL3 | sequence of CDRL3 | |
| | | of STIM007 using | |
| | | IMGT | |
| 473 | STIM007- | Amino acid | EIVLTQSPATLSLSPGERATLSCRASQSVTNY |
| | Light | sequence of V_{L} of | LAWHQQKPGQAPRLLIYDASNRATGIPARFS |
| | chain | STIM007 | GSGSGTDFTLTISSLEPEDFAVYYCQHRSNW |
| | variable | | PLTFGGGTKVEIK |
| | region | | |
| 474 | STIM007- | Nucleic acid | GAAATTGTATTGACACAGTCTCCAGCCAC |
| | Light | sequence of V_{L} of | CCTGTCTTTGTCTCCAGGGGAAAGAGCCA |
| | chain | STIM007 | CCCTCTCCTGCAGGGCCAGTCAGAGTGTT |
| | variable | | ACCAACTACTTAGCCTGGCACCAACAGAA |
| | region | | ACCTGGCCAGGCTCCCAGGCTCCTCATCT |
| | | | ATGATGCATCCAACAGGGCCACTGGCATC |
| | | | CCAGCCAGGTTCAGTGGCAGTGGGTCTGG |
| | | | GACAGACTTCACTCTCACCATCAGCAGCC |
| | | | TAGAGCCTGAAGATTTTGCAGTTTATTACT |
| | | | GTCAGCACCGTAGCAACTGGCCTCTCACT |
| | | | TTCGGCGGAGGGACCAAGGTGGAGATCAA |
| | | | AC |
| 475 | STIM007- | Amino acid | EIVLTQSPATLSLSPGERATLSCRASQSVTNY |
| | full | sequence of | LAWHQQKPGQAPRLLIYDASNRATGIPARFS |
| | light | STIM007 light chain | GSGSGTDFTLTISSLEPEDFAVYYCQHRSNW |
| | chain | | PLTFGGGTKVEIKRTVAAPSVFIFPPSDEQLK |
| | sequence | | SGTASVVCLLNNFYPREAKVQWKVDNALQ |
| | | | SGNSQESVTEQDSKDSTYSLSSTLTLSKADY |
| | | | EKHKVYACEVTHQGLSSPVTKSFNRGEC |
| 476 | STIM007- | Nucleic acid | GAAATTGTATTGACACAGTCTCCAGCCAC |
| | full | sequence of | CCTGTCTTTGTCTCCAGGGGAAAGAGCCA |
| | light | STIM007 light chain | CCCTCTCCTGCAGGGCCAGTCAGAGTGTT |
| | chain | | ACCAACTACTTAGCCTGGCACCAACAGAA |
| | sequence | | ACCTGGCCAGGCTCCCAGGCTCCTCATCT |
| | | | ATGATGCATCCAACAGGGCCACTGGCATC |
| | | | CCAGCCAGGTTCAGTGGCAGTGGGTCTGG |
| | | | GACAGACTTCACTCTCACCATCAGCAGCC |
| | | | TAGAGCCTGAAGATTTTGCAGTTTATTACT |
| | | | GTCAGCACCGTAGCAACTGGCCTCTCACT |
| | | | TTCGGCGGAGGGACCAAGGTGGAGATCAA |
| | | | ACcgtacggtggccgctccctccgtgttcatct |
| | | | tcccaccttccgacgagcagctgaagtccggca |
| | | | ccgcttctgtcgtgtgcctgctgaacaacttct |
| | | | acccccgcgaggccaaggtgcagtggaaggtgg |
| | | | acaacgccctgcagtccggcaactcccaggaat |
| | | | ccgtgaccgagcaggactccaaggacagcacct |
| | | | actccctgtcctccaccctgaccctgtccaagg |
| | | | ccgactacgagaagcacaaggtgtacgcctgcg |
| | | | aagtgacccaccagggcctgtctagccccgtga |
| | | | ccaagtctttcaaccggggcgagtgt |
| 477 | STIM008- | Amino acid | GFSLSTSGVG |
| | CDRH1 | sequence of CDRH1 | |
| | | of STIM008 using | |
| | | IMGT | |
| 478 | STIM008- | Amino acid | IYWDDDK |
| | CDRH2 | sequence of CDRH2 | |
| | | of STIM008 using | |
| | | IMGT | |
| 479 | STIM008- | Amino acid | THGYGSASYYHYGMDV |
| | CDRH3 | sequence of CDRH3 | |
| | | of STIM008 using | |
| | | IMGT | |
| 480 | STIM008- | Amino acid | QITLKESGPTLVKPTQTLTLTCTFSGFSLSTS |
| | Heavy | sequence of V_{H} of | GVGVGWIRQPPGKALEWLAVIYWDDDKRY |
| | chain | STIM008 | SPSLKSRLTITKDTSKNQVVLTMTNMDPVD |
| | variable | | TATYFCTHGYGSASYYHYGMDVWGQGTTV |
| | region | | TVSS |
| 481 | STIM008- | Nucleic acid | CAGATCACCTTGAAGGAGTCTGGTCCTAC |
| | Heavy | sequence of V_{H} of | GCTGGTGAAACCCACACAGACCCTCACGC |
| | chain | STIM008 | TGACCTGCACCTTCTCTGGGTTCTCACTCA |
| | variable | | GCACTAGTGGAGTGGGTGTGGGCTGGATC |
| | region | | CGTCAGCCCCCAGGAAAGGCCCTGGAGTG |
| | | | GCTTGCAGTCATTTATTGGGATGATGATA |
| | | | AGCGCTACAGCCCATCTCTGAAGAGCAGG |
| | | | CTCACCATCACCAAGGACACCTCCAAAAA |
| | | | CCAGGTGGTCCTTACAATGACCAACATGG |
| | | | ACCCTGTGGACACAGCCACATATTTCTGT |
| | | | ACACACGGATATGGTTCGGCGAGTTATTA |
| | | | CCACTACGGTATGGACGTCTGGGGCCAAG |
| | | | GGACCACGGTCACCGTCTCCTCA |
| 482 | STIM008- | Amino acid | QITLKESGPTLVKPTQTLTLTCTFSGFSLSTS |
| | full | sequence of | GVGVGWIRQPPGKALEWLAVIYWDDDKRY |
| | heavy | STIM008 heavy | SPSLKSRLTITKDTSKNQVVLTMTNMDPVD |
| | chain | chain | TATYFCTHGYGSASYYHYGMDVWGQGTTV |
| | sequence | | TVSSASTKGPSVFPLAPSSKSTSGGTAALGC |
| | | | LVKDYFPEPVTVSWNSGALTSGVHTFPAVL |
| | | | QSSGLYSLSSVVTVPSSSLGTQTYICNVNHK |
| | | | PSNTKVDKKVEPKSCDKTHTCPPCPAPELLG |
| | | | GPSVFLFPPKPKDTLMISRTPEVTCVVVDVS |
| | | | HEDPEVKFNWYVDGVEVHNAKTKPREEQY |
| | | | NSTYRVVSVLTVLHQDWLNGKEYKCKVSN |
| | | | KALPAPIEKTISKAKGQPREPQVYTLPPSRDE |
| | | | LTKNQVSLTCLVKGFYPSDIAVEWESNGQP |
| | | | ENNYKTTPPVLDSDGSFFLYSKLTVDKSRW |
| | | | QQGNVFSCSVMHEALHNHYTQKSLSLSPGK |
| 483 | STIM008- | Nucleic acid | CAGATCACCTTGAAGGAGTCTGGTCCTAC |
| | full | sequence of | GCTGGTGAAACCCACACAGACCCTCACGC |
| | heavy | STIM008 heavy | TGACCTGCACCTTCTCTGGGTTCTCACTCA |
| | chain | chain | GCACTAGTGGAGTGGGTGTGGGCTGGATC |
| | sequence | | CGTCAGCCCCCAGGAAAGGCCCTGGAGTG |
| | | | GCTTGCAGTCATTTATTGGGATGATGATA |
| | | | AGCGCTACAGCCCATCTCTGAAGAGCAGG |
| | | | CTCACCATCACCAAGGACACCTCCAAAAA |
| | | | CCAGGTGGTCCTTACAATGACCAACATGG |
| | | | ACCCTGTGGACACAGCCACATATTTCTGT |
| | | | ACACACGGATATGGTTCGGCGAGTTATTA |
| | | | CCACTACGGTATGGACGTCTGGGGCCAAG |
| | | | GGACCACGGTCACCGTCTCCTCAGCCAGC |
| | | | ACCAAGGGCCCCTCTGTGTTCCCTCTGGCC |
| | | | CCTTCCAGCAAGTCCACCTCTGGCGGAAC |
| | | | AGCCGCTCTGGGCTGCCTCGTGAAGGACT |
| | | | ACTTCCCCGAGCCTGTGACCGTGTCCTGG |
| | | | AACTCTGGCGCTCTGACCAGCGGAGTGCA |
| | | | CACCTTCCCTGCTGTGCTGCAGTCCTCCGG |
| | | | CCTGTACTCCCTGTCCTCCGTCGTGACCGT |
| | | | GCCTTCCAGCTCTCTGGGCACCCAGACCT |
| | | | ACATCTGCAACGTGAACCACAAGCCCTCC |
| | | | AACACCAAGGTGGACAAGAAGGTGGAAC |
| | | | CCAAGTCCTGCGACAAGACCCACACCTGT |
| | | | CCCCCTTGTCCTGCCCCTGAACTGCTGGGC |
| | | | GGACCTTCCGTGTTCCTGTTCCCCCCAAAG |
| | | | CCCAAGGACACCCTGATGATCTCCCGGAC |
| | | | CCCCGAAGTGACCTGCGTGGTGGTGGATG |
| | | | TGTCCCACGAGGACCCTGAAGTGAAGTTC |
| | | | AATTGGTACGTGGACGGCGTGGAAGTGCA |
| | | | CAACGCCAAGACCAAGCCTAGAGAGGAA |
| | | | CAGTACAACTCCACCTACCGGGTGGTGTC |
| | | | CGTGCTGACCGTGCTGCACCAGGATTGGC |
| | | | TGAACGGCAAAGAGTACAAGTGCAAGGT |
| | | | GTCCAACAAGGCCCTGCCTGCCCCCATCG |
| | | | AAAAGACCATCTCCAAGGCCAAGGGCCAG |
| | | | CCCCGGGAACCCCAGGTGTACACACTGCC |
| | | | CCCTAGCAGGGACGAGCTGACCAAGAACC |
| | | | AGGTGTCCCTGACCTGTCTCGTGAAAGGC |
| | | | TTCTACCCCTCCGATATCGCCGTGGAATGG |
| | | | GAGTCCAACGGCCAGCCTGAGAACAACTA |
| | | | CAAGACCACCCCCCCTGTGCTGGACTCCG |
| | | | ACGGCTCATTCTTCCTGTACAGCAAGCTG |
| | | | ACAGTGGACAAGTCCCGGTGGCAGCAGGG |
| | | | CAACGTGTTCTCCTGCTCCGTGATGCACGA |
| | | | GGCCCTGCACAACCACTACACCCAGAAGT |
| | | | CCCTGTCCCTGAGCCCCGGCAAGTGATGA |
| 484 | STIM008- | Amino acid | QSVTNY |
| | CDRL1 | sequence of CDRL1 | |
| | | of STIM008 using | |
| | | IMGT | |
| 485 | STIM008- | Amino acid | DAS |
| | CDRL2 | sequence of CDRL2 | |
| | | of STIM008 using | |
| | | IMGT | |
| 486 | STIM008- | Amino acid | QQRSNWPLT |
| | CDRL3 | sequence of CDRL3 | |
| | | of STIM008 using | |
| | | IMGT | |
| 487 | STIM008- | Amino acid | EIVLTQSPATLSLSPGERATLSCRASQSVTNY |
| | Light | sequence of V_{L} of | LAWHQQKPGQAPRLLIYDASNRATGIPARFS |
| | chain | STIM008 | GSGSGTDFTLTISSLEPEDFAVYYCQQRSNW |
| | variable | | PLTFGGGTKVEIK |
| | region | | |
| 488 | STIM008- | Nucleic acid | GAAATTGTGTTGACACAGTCTCCAGCCAC |
| | Light | sequence of V_{L} of | CCTGTCTTTGTCTCCAGGGGAAAGAGCCA |
| | chain | STIM008 | CCCTCTCCTGCAGGGCCAGTCAGAGTGTT |
| | variable | | ACCAACTACTTAGCCTGGCACCAACAGAA |
| | region | | ACCTGGCCAGGCTCCCAGGCTCCTCATCT |
| | | | ATGATGCATCCAACAGGGCCACTGGCATC |
| | | | CCAGCCAGGTTCAGTGGCAGTGGGTCTGG |
| | | | GACAGACTTCACTCTCACCATCAGCAGCC |
| | | | TAGAGCCTGAAGATTTTGCAGTTTATTACT |
| | | | GTCAGCAGCGTAGCAACTGGCCTCTCACT |
| | | | TTCGGCGGAGGGACCAAGGTGGAGATCAA |
| | | | A |
| 489 | STIM008- | Amino acid | EIVLTQSPATLSLSPGERATLSCRASQSVTNY |
| | full | sequence of | LAWHQQKPGQAPRLLIYDASNRATGIPARFS |
| | light | STIM008 light chain | GSGSGTDFTLTISSLEPEDFAVYYCQQRSNW |
| | chain | | PLTFGGGTKVEIKRTVAAPSVFIFPPSDEQLK |
| | sequence | | SGTASVVCLLNNFYPREAKVQWKVDNALQ |
| | | | SGNSQESVTEQDSKDSTYSLSSTLTLSKADY |
| | | | EKHKVYACEVTHQGLSSPVTKSFNRGEC |
| 490 | STIM008- | Nucleic acid | GAAATTGTGTTGACACAGTCTCCAGCCAC |
| | full | sequence of | CCTGTCTTTGTCTCCAGGGGAAAGAGCCA |
| | light | STIM008 light chain | CCCTCTCCTGCAGGGCCAGTCAGAGTGTT |
| | chain | | ACCAACTACTTAGCCTGGCACCAACAGAA |
| | sequence | | ACCTGGCCAGGCTCCCAGGCTCCTCATCT |
| | | | ATGATGCATCCAACAGGGCCACTGGCATC |
| | | | CCAGCCAGGTTCAGTGGCAGTGGGTCTGG |
| | | | GACAGACTTCACTCTCACCATCAGCAGCC |
| | | | TAGAGCCTGAAGATTTTGCAGTTTATTACT |
| | | | GTCAGCAGCGTAGCAACTGGCCTCTCACT |
| | | | TTCGGCGGAGGGACCAAGGTGGAGATCAA |
| | | | Acgtacggtggccgctccctccgtgttcatcttcc |
| | | | caccttccgacgagcagctgaagtccggcaccgct |
| | | | tctgtcgtgtgcctgctgaacaacttctacccccg |
| | | | cgaggccaaggtgcagtggaaggtggacaacgccct |
| | | | gcagtccggcaactcccaggaatccgtgaccgagca |
| | | | ggactccaaggacagcacctactccctgtcctccac |
| | | | cctgaccctgtccaaggccgactacgagaagcacaa |
| | | | ggtgtacgcctgcgaagtgacccaccagggcctgtc |
| | | | tagccccgtgaccaagtctttcaaccggggcgagtgt |
| 491 | STIM009- | Amino acid | GFTFSDYY |
| | CDRH1 | sequence of CDRH1 | |
| | | of STIM009 using | |
| | | IMGT | |
| 492 | STIM009- | Amino acid | ISSSGSTI |
| | CDRH2 | sequence of CDRH2 | |
| | | of STIM009 using | |
| | | IMGT | |
| 493 | STIM009- | Amino acid | ARDFYDILTDSPYFYYGVDV |
| | CDRH3 | sequence of CDRH3 | |
| | | of STIM009 using | |
| | | IMGT | |
| 494 | STIM009- | Amino acid | QVQLVESGGGLVKPGGSLRLSCAASGFTFS |
| | Heavy | sequence of V_{H} of | DYYMSWIRQAPGKGLEWVSYISSSGSTIYY |
| | chain | STIM009 | ADSVKGRFTISRDNAKNSLYLQINSLRAEDT |
| | variable | | AVYYCARDFYDILTDSPYFYYGVDVWGQG |
| | region | | TTVTVSS |
| 495 | STIM009- | Nucleic acid | CAGGTGCAGCTGGTGGAGTCTGGGGGAGG |
| | Heavy | sequence of V_{H} of | CTTGGTCAAGCCTGGAGGGTCCCTGAGAC |
| | chain | STIM009 | TCTCCTGTGCAGCCTCTGGATTCACCTTCA |
| | variable | | GTGACTACTACATGAGCTGGATCCGCCAG |
| | region | | GCTCCAGGGAAGGGGCTGGAGTGGGTTTC |
| | | | ATACATTAGTAGTAGTGGTAGTACCATAT |
| | | | ACTACGCAGACTCTGTGAAGGGCCGATTC |
| | | | ACCATCTCCAGGGACAACGCCAAGAACTC |
| | | | ACTGTATCTGCAAATTAACAGCCTGAGAG |
| | | | CCGAGGACACGGCCGTGTATTACTGTGCG |
| | | | AGAGATTTTTACGATATTTTGACTGATAGT |
| | | | CCGTACTTCTACTACGGTGTGGACGTCTGG |
| | | | GGCCAAGGGACCACGGTCACCGTCTCCTC |
| | | | A |
| 496 | STIM009- | Amino acid | QVQLVESGGGLVKPGGSLRLSCAASGFTFS |
| | full | sequence of | DYYMSWIRQAPGKGLEWVSYISSSGSTIYY |
| | heavy | STIM009 heavy | ADSVKGRFTISRDNAKNSLYLQINSLRAEDT |
| | chain | chain | AVYYCARDFYDILTDSPYFYYGVDVWGQG |
| | sequence | | TTVTVSSASTKGPSVFPLAPSSKSTSGGTAAL |
| | | | GCLVKDYFPEPVTVSWNSGALTSGVHTFPA |
| | | | VLQSSGLYSLSSVVTVPSSSLGTQTYICNVN |
| | | | HKPSNTKVDKKVEPKSCDKTHTCPPCPAPEL |
| | | | LGGPSVFLFPPKPKDTLMISRTPEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLPPSR |
| | | | DELTKNQVSLTCLVKGFYPSDIAVEWESNG |
| | | | QPENNYKTTPPVLDSDGSFFLYSKLTVDKSR |
| | | | WQQGNVFSCSVMHEALHNHYTQKSLSLSP |
| | | | GK |
| 497 | STIM009- | Nucleic acid | CAGGTGCAGCTGGTGGAGTCTGGGGGAGG |
| | full | sequence of | CTTGGTCAAGCCTGGAGGGTCCCTGAGAC |
| | heavy | STIM009 heavy | TCTCCTGTGCAGCCTCTGGATTCACCTTCA |
| | chain | chain | GTGACTACTACATGAGCTGGATCCGCCAG |
| | sequence | | GCTCCAGGGAAGGGGCTGGAGTGGGTTTC |
| | | | ATACATTAGTAGTAGTGGTAGTACCATAT |
| | | | ACTACGCAGACTCTGTGAAGGGCCGATTC |
| | | | ACCATCTCCAGGGACAACGCCAAGAACTC |
| | | | ACTGTATCTGCAAATTAACAGCCTGAGAG |
| | | | CCGAGGACACGGCCGTGTATTACTGTGCG |
| | | | AGAGATTTTTACGATATTTTGACTGATAGT |
| | | | CCGTACTTCTACTACGGTGTGGACGTCTGG |
| | | | GGCCAAGGGACCACGGTCACCGTCTCCTC |
| | | | AGCCAGCACCAAGGGCCCCTCTGTGTTCC |
| | | | CTCTGGCCCCTTCCAGCAAGTCCACCTCTG |
| | | | GCGGAACAGCCGCTCTGGGCTGCCTCGTG |
| | | | AAGGACTACTTCCCCGAGCCTGTGACCGT |
| | | | GTCCTGGAACTCTGGCGCTCTGACCAGCG |
| | | | GAGTGCACACCTTCCCTGCTGTGCTGCAGT |
| | | | CCTCCGGCCTGTACTCCCTGTCCTCCGTCG |
| | | | TGACCGTGCCTTCCAGCTCTCTGGGCACCC |
| | | | AGACCTACATCTGCAACGTGAACCACAAG |
| | | | CCCTCCAACACCAAGGTGGACAAGAAGGT |
| | | | GGAACCCAAGTCCTGCGACAAGACCCACA |
| | | | CCTGTCCCCCTTGTCCTGCCCCTGAACTGC |
| | | | TGGGCGGACCTTCCGTGTTCCTGTTCCCCC |
| | | | CAAAGCCCAAGGACACCCTGATGATCTCC |
| | | | CGGACCCCCGAAGTGACCTGCGTGGTGGT |
| | | | GGATGTGTCCCACGAGGACCCTGAAGTGA |
| | | | AGTTCAATTGGTACGTGGACGGCGTGGAA |
| | | | GTGCACAACGCCAAGACCAAGCCTAGAGA |
| | | | GGAACAGTACAACTCCACCTACCGGGTGG |
| | | | TGTCCGTGCTGACCGTGCTGCACCAGGAT |
| | | | TGGCTGAACGGCAAAGAGTACAAGTGCAA |
| | | | GGTGTCCAACAAGGCCCTGCCTGCCCCCA |
| | | | TCGAAAAGACCATCTCCAAGGCCAAGGGC |
| | | | CAGCCCCGGGAACCCCAGGTGTACACACT |
| | | | GCCCCCTAGCAGGGACGAGCTGACCAAGA |
| | | | ACCAGGTGTCCCTGACCTGTCTCGTGAAA |
| | | | GGCTTCTACCCCTCCGATATCGCCGTGGA |
| | | | ATGGGAGTCCAACGGCCAGCCTGAGAACA |
| | | | ACTACAAGACCACCCCCCCTGTGCTGGAC |
| | | | TCCGACGGCTCATTCTTCCTGTACAGCAAG |
| | | | CTGACAGTGGACAAGTCCCGGTGGCAGCA |
| | | | GGGCAACGTGTTCTCCTGCTCCGTGATGC |
| | | | ACGAGGCCCTGCACAACCACTACACCCAG |
| | | | AAGTCCCTGTCCCTGAGCCCCGGCAAGTG |
| | | | ATGA |
| 498 | STIM009- | Amino acid | QSLLHSNGYNY |
| | CDRL1 | sequence of CDRL1 | |
| | | of STIM009 using | |
| | | IMGT | |
| 499 | STIM009- | Amino acid | LGS |
| | CDRL2 | sequence of CDRL2 | |
| | | of STIM009 using | |
| | | IMGT | |
| 500 | STIM009- | Amino acid | MQALQTPRT |
| | CDRL3 | sequence of CDRL3 | |
| | | of STIM009 using | |
| | | IMGT | |
| 501 | STIM009- | Amino acid | DIVMTQSPLSLPVTPGEPASISCRSSQSLLHS |
| | Light | sequence of V_{L} of | NGYNYLDWYLQKPGQSPQLLIYLGSNRASG |
| | chain | STIM009 | VPDRFSGSGSGTDFTLKISRVEAEDVGVYYC |
| | variable | | MQALQTPRTFGQGTKVEIK |
| | region | | |
| 502 | STIM009- | Nucleic acid | GATATTGTGATGACTCAGTCTCCACTCTCC |
| | Light | sequence of V_{L} of | CTGCCCGTCACCCCTGGAGAGCCGGCCTC |
| | chain | STIM009 | CATCTCCTGCAGGTCTAGTCAGAGCCTCCT |
| | variable | | GCATAGTAATGGATACAACTATTTGGATT |
| | region | | GGTACCTGCAGAAGCCAGGGCAGTCTCCA |
| | | | CAGCTCCTGATCTATTTGGGTTCTAATCGG |
| | | | GCCTCCGGGGTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCTCGGACGTTCGGCCAAGGGACCA |
| | | | AGGTGGAAATCAAA |
| 503 | STIM009- | Amino acid | DIVMTQSPLSLPVTPGEPASISCRSSQSLLHS |
| | full | sequence of | NGYNYLDWYLQKPGQSPQLLIYLGSNRASG |
| | light | STIM009 light chain | VPDRFSGSGSGTDFTLKISRVEAEDVGVYYC |
| | chain | | MQALQTPRTFGQGTKVEIKRTVAAPSVFIFP |
| | sequence | | PSDEQLKSGTASVVCLLNNFYPREAKVQWK |
| | | | VDNALQSGNSQESVTEQDSKDSTYSLSSTLT |
| | | | LSKADYEKHKVYACEVTHQGLSSPVTKSFN |
| | | | RGEC |
| 504 | STIM009- | Nucleic acid | GATATTGTGATGACTCAGTCTCCACTCTCC |
| | full | sequence of | CTGCCCGTCACCCCTGGAGAGCCGGCCTC |
| | light | STIM009 light chain | CATCTCCTGCAGGTCTAGTCAGAGCCTCCT |
| | chain | | GCATAGTAATGGATACAACTATTTGGATT |
| | sequence | | GGTACCTGCAGAAGCCAGGGCAGTCTCCA |
| | | | CAGCTCCTGATCTATTTGGGTTCTAATCGG |
| | | | GCCTCCGGGGTCCCTGACAGGTTCAGTGG |
| | | | CAGTGGATCAGGCACAGATTTTACACTGA |
| | | | AAATCAGCAGAGTGGAGGCTGAGGATGTT |
| | | | GGGGTTTATTACTGCATGCAAGCTCTACA |
| | | | AACTCCTCGGACGTTCGGCCAAGGGACCA |
| | | | AGGTGGAAATCAAAcgtacggtggccgctccctccgtg |
| | | | ttcatcttcccaccttccgacgagcagctgaagtccgg |
| | | | caccgcttctgtcgtgtgcctgctgaacaacttctacc |
| | | | cccgcgaggccaaggtgcagtggaaggtggacaacgcc |
| | | | ctgcagtccggcaactcccaggaatccgtgaccgagca |
| | | | ggactccaaggacagcacctactccctgtcctccaccc |
| | | | tgaccctgtccaaggccgactacgagaagcacaaggtg |
| | | | tacgcctgcgaagtgacccaccagggcctgtctagccc |
| | | | cgtgaccaagtctttcaaccggggcgagtgt |
| 505 | Human | Amino acid | FTVTVPKDLYVVEYGSNMTIECKFPVEKQL |
| | PD-L1 | sequence of | DLAALIVYWEMEDKNIIQFVHGEEDLKVQH |
| | Flag His | KYPROT286 with | SSYRQRARLLKDQLSLGNAALQITDVKLQD |
| | (KYPROT286) | FLAG tag in bold | AGVYRCMISYGGADYKRITVKVNAPYNKIN |
| | | and underlined and | QRILVVDPVTSEHELTCQAEGYPKAEVIWTS |
| | | histidine tag in bold | SDHQVLSGKTTTTNSKREEKLFNVTSTLRIN |
| | | | TTTNEIFYCTFRRLDPEENHTAELVIPELPLA |
| | | | HPPNERTIEGRDYKDDDDKHHHHHH |
| 506 | Mature | Mature amino acid | EINGSANYEMFIFHNGGVQILCKYPDIVQQF |
| | human | sequence of human | KMQLLKGGQILCDLTKTKGSGNTVSIKSLKF |
| | ICOS | ICOS | CHSQLSNNSVSFFLYNLDHSHANYYFCNLSI |
| | | | FDPPPFKVTLTGGYLHIYESQLCCQLKFWLPI |
| | | | GCAAFVVVCILGCILICWLTKKKYSSSVHDP |
| | | | NGEYMFMRAVNTAKKSRLTDVTL |
| 507 | Human | Amino acid | EINGSANYEMFIFHNGGVQILCKYPDIVQQF |
| | ICOS | sequence of human | KMQLLKGGQILCDLTKTKGSGNTVSIKSLKF |
| | extra- | ICOS extracellular | CHSQLSNNSVSFFLYNLDHSHANYYFCNLSI |
| | cellular | domain | FDPPPFKVTLTGGYLHIYESQLCCQLKF |
| | domain | | |
| 508 | Human | Amino acid | MKSGLWYFFLFCLRIKVLTGEINGSANYEM |
| | ICOS | sequence of human | FIFHNGGVQILCKYPDIVQQFKMQLLKGGQI |
| | with | ICOS (signal peptide | LCDLTKTKGSGNTVSIKSLKFCHSQLSNNSV |
| | signal | is underlined) | SFFLYNLDHSHANYYFCNLSIFDPPPFKVTLT |
| | peptide | | GGYLHIYESQLCCQLKFWLPIGCAAFVVVCI |
| | | | LGCILICWLTKKKYSSSVHDPNGEYMFMRA |
| | | | VNTAKKSRLTDVTL |
| 509 | Isoform | Amino acid | The sequence of this isoform |
| | of human | sequence of a human | differs from the canonical |
| | ICOS | ICOS isoform | sequence in its cytoplasmic |
| | (Q9Y6W8-2) | | domain as follows: 168-199: |
| | | | KYSSSVHDPNGEYMFMRAVNTAKKSRLTD |
| | | | VTLM |
| 510 | Mature | Mature amino acid | EINGSADHRMFSFHNGGVQISCKYPETVQQL |
| | mouse | sequence of mouse | KMRLFREREVLCELTKTKGSGNAVSIKNPM |
| | ICOS | ICOS | LCLYHLSNNSVSFFLNNPDSSQGSYYFCSLSI |
| | | | FDPPPFQERNLSGGYLHIYESQLCCQLKIVV |
| | | | QVTE |
| 511 | Mouse | Amino acid | EINGSADHRMFSFHNGGVQISCKYPETVQQL |
| | ICOS | sequence of the | KMRLFREREVLCELTKTKGSGNAVSIKNPM |
| | extra- | extracellular domain | LCLYHLSNNSVSFFLNNPDSSQGSYYFCSLSI |
| | cellular | of mouse ICOS | FDPPPFQERNLSGGYLHIYESQLCCQLK |
| | domain | | |
| 512 | Mouse | Amino acid | MGWSCIILFLVATATGVHSEINGSADHRMFS |
| | with | sequence of mouse | FHNGGVQISCKYPETVQQLKMRLFREREVL |
| | signal | ICOS (signal peptide | CELTKTKGSGNAVSIKNPMLCLYHLSNNSV |
| | peptide | is underlined) | SFFLNNPDSSQGSYYFCSLSIFDPPPFQERNLS |
| | ICOS | | GGYLHIYESQLCCQLKIVVQVTE |
| 513 | Cynomolgus | Amino acid | MKSGLWYFFLFCLHMKVLTG |
| | ICOS with | sequence of | EINGSANYEM FIFHNGGVQI LCKYPDIVQQ |
| | signal | cynomolgus ICOS | FKMQLLKGGQILCDLTKTKGSGNKVSIKSL |
| | peptide | (signal peptide is | KFCHSQLSNNSVSFFLYNLD |
| | | underlined) | RSHANYYFCNLSIFDPPPFKVTLTGGYLHIYE |
| | | | SQLCCQLKFWLPIGCATF |
| | | | VVVCIFGCILICWLTKKKYSSTVHDPNGEYM |
| | | | FMRAVNTAKKSRLTGTTP |
| 514 | Cynomolgus | Amino acid | EINGSANYEMFIFHNGGVQILCKYPDIVQQF |
| | ICOS | sequence of | KMQLLKGGQILCDLTKTKG |
| | extra- | cynomolgus ICOS | SGNKVSIKSLKFCHSQLSNNSVSFFLYNLDR |
| | cellular | extracellular domain | SHANYYFCNLSIFDPPPFK |
| | domain | | VTLTGGYLHIYESQLCCQLK |
| 515 | Human | Amino acid | DTQEKEVRAMVGSDVELSCACPEGSRFDLN |
| | ICOS | sequence of human | DVYVYWQTSESKTVVTYHIPQNSSLENVDS |
| | ligand | ICOS ligand | RYRNRALMSPAGMLRGDFSLRLFNVTPQDE |
| | | comprising | QKFHCLVLSQSLGFQEVLSVEVTLHVAANF |
| | | extracellular domain | SVPVVSAPHSPSQDELTFTCTSINGYPRPNV |
| | | | YWINKTDNSLLDQALQNDTVFLNMRGLYDV |
| | | | VSVLRIARTPSVNIGCCIENVLLQQNLTVGS |
| | | | QTGNDIGERDKITENPVSTGEKNAATWS |
| 516 | Human | Amino acid | MRLGSPGLLFLLFSSLRADTQEKEVRAMVG |
| | ICOS | sequence of human | SDVELSCACPEGSRFDLNDVYVYWQTSESK |
| | ligand | ICOS ligand | TVVTYHIPQNSSLENVDSRYRNRALMSPAG |
| | | including signal | MLRGDFSLRLFNVTPQDEQKFHCLVLSQSL |
| | | peptide | GFQEVLSVEVTLHVAANFSVPVVSAPHSPSQ |
| | | | DELTFTCTSINGYPRPNVYWINKTDNSLLDQ |
| | | | ALQNDTVFLNMRGLYDVVSVLRIARTPSVN |
| | | | IGCCIENVLLQQNLTVGSQTGNDIGERDKITE |
| | | | NPVSTGEKNAATWSILAVLCLLVVVAVAIG |
| | | | WVCRDRCLQHSYAGAWAVSPETELTGHV |

1.21.1.12.1 SEQ ID NO: 610 ICOSL-Fc
DTQEKEVRAMVGSDVELSCACPEGSRFDLNDVYVYWQTSESKTVVTYHIPQNSSLE
NVDSRYRNRALMSPAGMLRGDFSLRLFNVTPQDEQKFHCLVLSQSLGFQEVLSVEV
TLHVAANFSVPVVSAPHSPSQDELTFTCTSINGYPRPNVYWINKTDNSLLDQALQND
TVFLNMRGLYDVVSVLRIARTPSVNIGCCIENVLLQQNLTVGSQTGNDIGERDKITEN
PVSTGEKNAATWSDIEGRMDPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNV
FSCSVMHEALHNHYTQKSLSLSPGK
Linker is underlined and in bold. Sequence preceding linker is human ICOSL (B7-H2).
Sequence following linker is human IgG1 Fc.

| 517 | C-terminal amino | | Amino acids 21 to | LQMILNGINNYKNPKLTAMLTFKFY |
|---|---|---|---|---|
| | acid sequence of | | 133 of hIL-2 with | MPKKATELKHLQCLEEELKPLEEVLN |
| | hIL-2 | | R38W mutation | LAQSKNFHLRPRDLISNINVIVLELK |
| | | | (bold & | GSETTFMCEYADETATIVEFLNRWIT |
| | | | underlined) | FCQSIISTLT |
| 518 | C-terminal amino | | Amino acids 21 to | LQMILNGINNYKNPKLTQMLTFKFY |
| | acid sequence of | | 133 of hIL-2 with | MPKKATELKHLQCLEEELKPLEEVLN |
| | hIL-2 | | R38Q mutation | LAQSKNFHLRPRDLISNINVIVLELKG |
| | | | (bold & | SETTFMCEYADETATIVEFLNRWITFC |
| | | | underlined) | QSIISTLT |
| 519 | STIM002- | | Nucleic acid | GATATTGTGATGACTCAGTCTCCAC |
| | Corrected Light | | sequence of | TCTCCCTGCCCGTCACCCCTGGAGA |
| | chain variable | | corrected V_{L} of | GCCGGCCTCCATCTCCTGCAGGTCT |
| | region | | STIM002 | AGTCAGAGCCTCCTGCATAGTGATG |
| | | | | GATACAACTATTTGGATTGGTACCT |
| | | | | GCAGAAGCCAGGGCAGTCTCCACAG |
| | | | | CTCCTGATCTATTTGGGTTCTACTC |
| | | | | GGGCCTCCGGGTTCCCTGACAGGTT |
| | | | | CAGTGGCAGTGGATCAGGCACAGA |
| | | | | TTTTACACTGAAAATCAGCAGAGTG |
| | | | | GAGGCTGAGGATGTTGGGGTTTATT |
| | | | | ACTGCATGCAAGCTCTACAAACTCC |
| | | | | GCTCAGTTTTGGCCAGGGGACCAAG |
| | | | | CTGGAGATCAAA |
| 520 | STIM002- | | Nucleic acid | GATATTGTGATGACTCAGTCTCCAC |
| | Corrected full | | sequence of | TCTCCCTGCCCGTCACCCCTGGAGA |
| | light chain | | corrected STIM002 | GCCGGCCTCCATCTCCTGCAGGTCT |
| | sequence | | light chain | AGTCAGAGCCTCCTGCATAGTGATG |
| | | | | GATACAACTATTTGGATTGGTACCT |
| | | | | GCAGAAGCCAGGGCAGTCTCCACAG |
| | | | | CTCCTGATCTATTTGGGTTCTACTC |
| | | | | GGGCCTCCGGGTTCCCTGACAGGTT |
| | | | | CAGTGGCAGTGGATCAGGCACAGA |
| | | | | TTTTACACTGAAAATCAGCAGAGTG |
| | | | | GAGGCTGAGGATGTTGGGGTTTATT |
| | | | | ACTGCATGCAAGCTCTACAAACTCC |
| | | | | GCTCAGTTTTGGCCAGGGGACCAAG |
| | | | | CTGGAGATCAAAcgtacggtggccgct |
| | | | | ccctccgtgttcatcttcccaccttcc |
| | | | | gacgagcagctgaagtccggcaccgct |
| | | | | tctgtcgtgtgcctgctgaacaacttc |
| | | | | tacccccgcgaggccaaggtgcagtgg |
| | | | | aaggtggacaacgccctgcagtccggc |
| | | | | aactcccaggaatccgtgaccgagcag |
| | | | | gactccaaggacagcacctactccctg |
| | | | | tcctccaccctgaccctgtccaaggcc |
| | | | | gactacgagaagcacaaggtgtacgcc |
| | | | | tgcgaagtgacccaccagggcctgtct |
| | | | | agccccgtgaccaagtctttcaaccgg |
| | | | | ggcgagtgt |
| 521 | STIM003- | | Nucleic acid | GAGGTGCAGCTGGTGGAGTCTGGG |
| | Corrected heavy | | sequence of | GGAGGTGTGGTACGGCCTGGGGGG |
| | chain variable | | corrected V_{H} of | TCCCTGAGACTCTCCTGTGTAGCCT |
| | region | | STIM003 | CTGGAGTCACCTTTGATGATTATGG |
| | | | | CATGAGCTGGGTCCGCCAAGCTCCA |
| | | | | GGGAAGGGGCTGGAGTGGGTCTCT |
| | | | | GGTATTAATTGGAATGGTGGCGACA |
| | | | | CAGATTATTCAGACTCTGTGAAGGG |
| | | | | CCGATTCACCATCTCCAGAGACAAC |
| | | | | GCCAAGAACTCCCTGTATCTACAAA |
| | | | | TGAATAGTCTGAGAGCCGAGGACA |
| | | | | CGGCCTTGTATTACTGTGCGAGGGA |
| | | | | TTTCTATGGTTCGGGGAGTTATTATC |
| | | | | ACGTTCCTTTTGACTACTGGGGCCA |
| | | | | GGGAATCCTGGTCACCGTCTCCTCA |
| 522 | STIM003- | | Nucleic acid | GAGGTGCAGCTGGTGGAGTCTGGG |
| | Corrected full | | sequence of | GGAGGTGTGGTACGGCCTGGGGGG |
| | heavy chain | | corrected STIM003 | TCCCTGAGACTCTCCTGTGTAGCCT |
| | sequence | | heavy chain | CTGGAGTCACCTTTGATGATTATGG |
| | | | | CATGAGCTGGGTCCGCCAAGCTCCA |
| | | | | GGGAAGGGGCTGGAGTGGGTCTCT |
| | | | | GGTATTAATTGGAATGGTGGCGACA |
| | | | | CAGATTATTCAGACTCTGTGAAGGG |
| | | | | CCGATTCACCATCTCCAGAGACAAC |
| | | | | GCCAAGAACTCCCTGTATCTACAAA |
| | | | | TGAATAGTCTGAGAGCCGAGGACA |
| | | | | CGGCCTTGTATTACTGTGCGAGGGA |
| | | | | TTTCTATGGTTCGGGGAGTTATTATC |
| | | | | ACGTTCCTTTTGACTACTGGGGCCA |
| | | | | GGGAATCCTGGTCACCGTCTCCTCA |
| | | | | GCCAGCACCAAGGGCCCCTCTGTGT |
| | | | | TCCCTCTGGCCCCTTCCAGCAAGTC |
| | | | | CACCTCTGGCGGAACAGCCGCTCTG |
| | | | | GGCTGCCTCGTGAAGGACTACTTCC |
| | | | | CCGAGCCTGTGACCGTGTCCTGGAA |
| | | | | CTCTGGCGCTCTGACCAGCGGAGTG |
| | | | | CACACCTTCCCTGCTGTGCTGCAGT |
| | | | | CCTCCGGCCTGTACTCCCTGTCCTCC |
| | | | | GTCGTGACCGTGCCTTCCAGCTCTC |
| | | | | TGGGCACCCAGACCTACATCTGCAA |
| | | | | CGTGAACCACAAGCCCTCCAACACC |
| | | | | AAGGTGGACAAGAAGGTGGAACCC |
| | | | | AAGTCCTGCGACAAGACCCACACCT |
| | | | | GTCCCCCTTGTCCTGCCCCTGAACT |
| | | | | GCTGGGCGGACCTTCCGTGTTCCTG |
| | | | | TTCCCCCCAAAGCCCAAGGACACCC |
| | | | | TGATGATCTCCCGGACCCCCGAAGT |
| | | | | GACCTGCGTGGTGGTGGATGTGTCC |
| | | | | CACGAGGACCCTGAAGTGAAGTTCA |
| | | | | ATTGGTACGTGGACGGCGTGGAAGT |
| | | | | GCACAACGCCAAGACCAAGCCTAG |
| | | | | AGAGGAACAGTACAACTCCACCTAC |
| | | | | CGGGTGGTGTCCGTGCTGACCGTGC |
| | | | | TGCACCAGGATTGGCTGAACGGCAA |
| | | | | AGAGTACAAGTGCAAGGTGTCCAA |
| | | | | CAAGGCCCTGCCTGCCCCCATCGAA |
| | | | | AAGACCATCTCCAAGGCCAAGGGC |
| | | | | CAGCCCCGGGAACCCCAGGTGTACA |
| | | | | CACTGCCCCCTAGCAGGGACGAGCT |
| | | | | GACCAAGAACCAGGTGTCCCTGACC |
| | | | | TGTCTCGTGAAAGGCTTCTACCCCT |
| | | | | CCGATATCGCCGTGGAATGGGAGTC |
| | | | | CAACGGCCAGCCTGAGAACAACTA |
| | | | | CAAGACCACCCCCCCTGTGCTGGAC |
| | | | | TCCGACGGCTCATTCTTCCTGTACA |
| | | | | GCAAGCTGACAGTGGACAAGTCCC |
| | | | | GGTGGCAGCAGGGCAACGTGTTCTC |
| | | | | CTGCTCCGTGATGCACGAGGCCCTG |
| | | | | CACAACCACTACACCCAGAAGTCCC |
| | | | | TGTCCCTGAGCCCCGGCAAGTGATG |
| | | | | A |
| 523 | Human | IGHG1* | Human Heavy | gcctccaccaagggcccatcggtcttccccctggcaccct |
| | IgG1 | 03 | Chain Constant | cctccaagagcacctctgggggcacagcggccctgggct |
| | constant | | Region | gcctggtcaaggactacttccccgaaccggtgacggtgtc |
| | region | | (IGHG1*03) | gtggaactcaggcgccctgaccagcggcgtgcacacctt |
| | | | Nucleotide | cccggctgtcctacagtcctcaggactctactccctcagca |
| | | | Sequence | gcgtggtgaccgtgccctccagcagcttgggcacccaga |
| | | | | cctacatctgcaacgtgaatcacaagcccagcaacaccaa |
| | | | | ggtggacaagagagttgagcccaaatcttgtgacaaaact |
| | | | | cacacatgcccaccgtgcccagcacctgaactcctgggg |
| | | | | ggaccgtcagtcttcctcttccccccaaaacccaaggaca |
| | | | | ccctcatgatctcccggacccctgaggtcacatgcgtggt |
| | | | | ggtggacgtgagccacgaagaccctgaggtcaagttcaa |
| | | | | ctggtacgtggacggcgtggaggtgcataatgccaagac |
| | | | | aaagccgcgggaggagcagtacaacagcacgtaccgtg |
| | | | | tggtcagcgtcctcaccgtcctgcaccaggactggctgaa |
| | | | | tggcaaggagtacaagtgcaaggtctccaacaaagccct |
| | | | | cccagcccccatcgagaaaaccatctccaaagccaaagg |
| | | | | gcagccccgagaaccacaggtgtacaccctgcccccatc |
| | | | | ccgggaggagatgaccaagaaccaggtcagcctgacct |
| | | | | gcctggtcaaaggcttctatcccagcgacatcgccgtgga |
| | | | | gtgggagagcaatgggcagccggagaacaactacaaga |
| | | | | ccacgcctcccgtgctggactccgacggctccttcttcctct |
| | | | | atagcaagctcaccgtggacaagagcaggtggcagcag |
| | | | | gggaacgtcttctcatgctccgtgatgcatgaggctctgca |
| | | | | caaccactacacgcagaagagcctctccctgtccccgggt |
| | | | | aaa |
| 524 | | | Human Heavy | A S T K G P S V F P L A P S S K S T S G |
| | | | Chain Constant | G T A A L G C L V K D Y F P E P V T V |
| | | | Region | S W N S G A L T S G V H T F P A V L Q |
| | | | (IGHG1*03) | S S G L Y S L S S V V T V P S S S L G T |
| | | | Protein | Q T Y I C N V N H K P S N T K V D K R |
| | | | Sequence | V E P K S C D K T H T C P P C P A P E |
| | | | | L L G G P S V F L F P P K P K D T L M I |
| | | | | S R T P E V T C V V V D V S H E D P E |
| | | | | V K F N W Y V D G V E V H N A K T K |
| | | | | P R E E Q Y N S T Y R V V S V L T V L |
| | | | | H Q D W L N G K E Y K C K V S N K A |
| | | | | L P A P I E K T I S K A K G Q P R E P Q |
| | | | | V Y T L P P S R E E M T K N Q V S L T |
| | | | | C L V K G F Y P S D I A V E W E S N G |
| | | | | Q P E N N Y K T T P P V L D S D G S F |
| | | | | F L Y S K L T V D K S R W Q Q G N V |
| | | | | F S C S V M H E A L H N H Y T Q K S L |
| | | | | S L S P G K |
| 525 | Human | IGHG1* | Human Heavy | gcctccaccaagggcccatcggtcttccccctggcaccct |
| | IgG1 | 04 | Chain Constant | cctccaagagcacctctgggggcacagcggccctgggct |
| | constant | | Region | gcctggtcaaggactacttccccgaaccggtgacggtgtc |
| | region | | (IGHG1*04) | gtggaactcaggcgccctgaccagcggcgtgcacacctt |
| | | | Nucleotide | cccggctgtcctacagtcctcaggactctactccctcagca |
| | | | Sequence | gcgtggtgaccgtgccctccagcagcttgggcacccaga |
| | | | | cctacatctgcaacgtgaatcacaagcccagcaacaccaa |
| | | | | ggtggacaagaaagttgagcccaaatcttgtgacaaaact |
| | | | | cacacatgcccaccgtgcccagcacctgaactcctgggg |
| | | | | ggaccgtcagtcttcctcttccccccaaaacccaaggaca |
| | | | | ccctcatgatctcccggacccctgaggtcacatgcgtggt |
| | | | | ggtggacgtgagccacgaagaccctgaggtcaagttcaa |
| | | | | ctggtacgtggacggcgtggaggtgcataatgccaagac |
| | | | | aaagccgcgggaggagcagtacaacagcacgtaccgtg |
| | | | | tggtcagcgtcctcaccgtcctgcaccaggactggctgaa |
| | | | | tggcaaggagtacaagtgcaaggtctccaacaaagccct |
| | | | | cccagcccccatcgagaaaaccatctccaaagccaaagg |
| | | | | gcagccccgagaaccacaggtgtacaccctgcccccatc |
| | | | | ccgggatgagctgaccaagaaccaggtcagcctgacctg |
| | | | | cctggtcaaaggcttctatcccagcgacatcgccgtggag |
| | | | | tgggagagcaatgggcagccggagaacaactacaagacca |
| | | | | cgcctcccgtgctggactccgacggctccttcttcctcta |
| | | | | cagcaagctcaccgtggacaagagcaggtggcagcagggg |
| | | | | aacatcttctcatgctccgtgatgcatgaggctctgcac |
| | | | | aaccactacacgcagaagagcctctccctgtctccgggta |
| | | | | aa |
| 526 | | | Human Heavy | ASTKGPSVFPLAPSSKSTSGGTAALGC |
| | | | Chain Constant | LVKDYFPEPVTVSWNSGALTSGVHTF |
| | | | Region | PAVLQSSGLYSLSSVVTVPSSSLGTQT |
| | | | (IGHG1*04) | YICNVNHKPSNTKVDKKVEPKSCDKT |
| | | | Protein Sequence | HTCPPCPAPELLGGPSVFLFPPKPKDT |
| | | | | LMISRTPEVTCVVVDVSHEDPEVKFN |
| | | | | WYVDGVEVHNAKTKPREEQYNSTYR |
| | | | | VVSVLTVLHQDWLNGKEYKCKVSN |
| | | | | KALPAPIEKTISKAKGQPREPQVYTLP |
| | | | | PSRDELTKNQVSLTCLVKGFYPSDIA |
| | | | | VEWESNGQPENNYKTTPPVLDSDGSF |
| | | | | FLYSKLTVDKSRWQQGNIFSCSVMHE |
| | | | | ALHNHYTQKSLSLSPGK |
| 527 | Human | IGHG2* | Human Heavy | gcctccaccaagggcccatcggtcttccccctggcgccct |
| | IgG2 | 01 | Chain Constant | gctccaggagcacctccgagagcacagccgccctgggc |
| | constant | & | Region | tgcctggtcaaggactacttccccgaaccggtgacggtgt |
| | region | IGHG2* | (IGHG2*01) | cgtggaactcaggcgctctgaccagcggcgtgcacacctt |
| | | 03 | Nucleotide | cccagctgtcctacagtcctcaggactctactccctcagca |
| | | & | Sequence | gcgtggtgaccgtgccctccagcaacttcggcacccaga |
| | | IGHG2* | | cctacacctgcaacgtagatcacaagcccagcaacacca |
| | | 05 | | aggtggacaagacagttgagcgcaaatgttgtgtcgagtg |
| | | | | cccaccgtgcccagcaccacctgtggcaggaccgtcagtc |
| | | | | ttcctcttccccccaaaacccaaggacaccctcatgatct |
| | | | | cccggacccctgaggtcacgtgcgtggtggtggacgtgag |
| | | | | ccacgaagaccccgaggtccagttcaactggtacgtgga |
| | | | | cggcgtggaggtgcataatgccaagacaaagccacggg |
| | | | | aggagcagttcaacagcacgttccgtgtggtcagcgtcct |
| | | | | caccgttgtgcaccaggactggctgaacggcaaggagta |
| | | | | caagtgcaaggtctccaacaaaggcctcccagcccccat |
| | | | | cgagaaaaccatctccaaaaccaaagggcagccccgag |
| | | | | aaccacaggtgtacaccctgcccccatcccgggaggaga |
| | | | | tgaccaagaaccaggtcagcctgacctgcctggtcaaag |
| | | | | gcttctaccccagcgacatcgccgtggagtgggagagca |
| | | | | atgggcagccggagaacaactacaagaccacacctccca |
| | | | | tgctggactccgacggctccttcttcctctacagcaagct |
| | | | | caccgtggacaagagcaggtggcagcaggggaacgtcttc |
| | | | | tcatgctccgtgatgcatgaggctctgcacaaccactaca |
| | | | | cgcagaagagcctctccctgtctccgggtaaa |
| 528 | | | Human Heavy | ASTKGPSVFPLAPCSRSTSESTAALGC |
| | | | Chain Constant | LVKDYFPEPVTVSWNSGALTSGVHTF |
| | | | Region | PAVLQSSGLYSLSSVVTVPSSNFGTQT |
| | | | (IGHG2*01) | YTCNVDHKPSNTKVDKTVERKCCVE |
| | | | Protein Sequence | CPPCPAPPVAGPSVFLFPPKPKDTLMI |
| | | | | SRTPEVTCVVVDVSHEDPEVQFNWY |
| | | | | VDGVEVHNAKTKPREEQFNSTFRVVS |
| | | | | VLTVVHQDWLNGKEYKCKVSNKGL |
| | | | | PAPIEKTISKTKGQPREPQVYTLPPSRE |
| | | | | EMTKNQVSLTCLVKGFYPSDIAVEWE |
| | | | | SNGQPENNYKTTPPMLDSDGSFFLYS |
| | | | | KLTVDKSRWQQGNVFSCSVMHEALH |
| | | | | NHYTQKSLSLSPGK |
| 529 | Human | IGHG2* | Human Heavy | GCCTCCACCAAGGGCCCATCGGTCT |
| | IgG2 | 02 | Chain Constant | TCCCCCTGGCGCCCTGCTCCAGGAG |
| | constant | | Region | CACCTCCGAGAGCACAGCGGCCCTG |
| | region | | (IGHG2*02) | GGCTGCCTGGTCAAGGACTACTTCC |
| | | | Nucleotide | CCGAACCGGTGACGGTGTCGTGGAA |
| | | | Sequence | CTCAGGCGCTCTGACCAGCGGCGTG |
| | | | | CACACCTTCCCGGCTGTCCTACAGT |
| | | | | CCTCAGGACTCTACTCCCTCAGCAG |
| | | | | CGTGGTGACCGTGACCTCCAGCAAC |
| | | | | TTCGGCACCCAGACCTACACCTGCA |
| | | | | ACGTAGATCACAAGCCCAGCAACA |
| | | | | CCAAGGTGGACAAGACAGTTGAGC |
| | | | | GCAAATGTTGTGTCGAGTGCCCACC |
| | | | | GTGCCCAGCACCACCTGTGGCAGGA |
| | | | | CCGTCAGTCTTCCTCTTCCCCCCAAA |
| | | | | ACCCAAGGACACCCTCATGATCTCC |
| | | | | CGGACCCCTGAGGTCACGTGCGTGG |
| | | | | TGGTGGACGTGAGCCACGAAGACC |
| | | | | CCGAGGTCCAGTTCAACTGGTACGT |
| | | | | GGACGGCATGGAGGTGCATAATGC |
| | | | | CAAGACAAAGCCACGGGAGGAGCA |
| | | | | GTTCAACAGCACGTTCCGTGTGGTC |
| | | | | AGCGTCCTCACCGTCGTGCACCAGG |
| | | | | ACTGGCTGAACGGCAAGGAGTACA |
| | | | | AGTGCAAGGTCTCCAACAAAGGCCT |
| | | | | CCCAGCCCCCATCGAGAAAACCATC |
| | | | | TCCAAAACCAAAGGGCAGCCCCGA |
| | | | | GAACCACAGGTGTACACCCTGCCCC |
| | | | | CATCCCGGGAGGAGATGACCAAGA |
| | | | | ACCAGGTCAGCCTGACCTGCCTGGT |
| | | | | CAAAGGCTTCTACCCCAGCGACATC |
| | | | | GCCGTGGAGTGGGAGAGCAATGGG |
| | | | | CAGCCGGAGAACAACTACAAGACC |
| | | | | ACACCTCCCATGCTGGACTCCGACG |
| | | | | GCTCCTTCTTCCTCTACAGCAAGCTC |
| | | | | ACCGTGGACAAGAGCAGGTGGCAG |
| | | | | CAGGGGAACGTCTTCTCATGCTCCG |
| | | | | TGATGCATGAGGCTCTGCACAACCA |
| | | | | CTACACACAGAAGAGCCTCTCCCTG |
| | | | | TCTCCGGGTAAA |
| 530 | | | Human Heavy | ASTKGPSVFPLAPCSRSTSESTAALGC |
| | | | Chain Constant | LVKDYFPEPVTVSWNSGALTSGVHTF |
| | | | Region | PAVLQSSGLYSLSSVVTVTSSNFGTQT |
| | | | (IGHG2*02) | YTCNVDHKPSNTKVDKTVERKCCVE |
| | | | Protein Sequence | CPPCPAPPVAGPSVFLFPPKPKDTLMI |
| | | | | SRTPEVTCVVVDVSHEDPEVQFNWY |
| | | | | VDGMEVHNAKTKPREEQFNSTFRVV |
| | | | | SVLTVVHQDWLNGKEYKCKVSNKG |
| | | | | LPAPIEKTISKTKGQPREPQVYTLPPSR |
| | | | | EEMTKNQVSLTCLVKGFYPSDIAVEW |
| | | | | ESNGQPENNYKTTPPMLDSDGSFFLY |
| | | | | SKLTVDKSRWQQGNVFSCSVMHEAL |
| | | | | HNHYTQKSLSLSPGK |
| 531 | Human | IGHG2* | Human Heavy | gcctccaccaagggcccatcggtcttccccctggcgccct |
| | IgG2 | 04 | Chain Constant | gctccaggagcacctccgagagcacagcggccctgggc |
| | constant | | Region | tgcctggtcaaggactacttccccgaaccggtgacggtgt |
| | region | | (IGHG2*04) | cgtggaactcaggcgctctgaccagcggcgtgcacacctt |
| | | | Nucleotide | cccagctgtcctacagtcctcaggactctactccctcagca |
| | | | Sequence | gcgtggtgaccgtgccctccagcagcttgggcacccaga |
| | | | | cctacacctgcaacgtagatcacaagcccagcaacacca |
| | | | | aggtggacaagacagttgagcgcaaatgttgtgtcgagtg |
| | | | | cccaccgtgcccagcaccacctgtggcaggaccgtcagt |
| | | | | cttcctcttccccccaaaacccaaggacaccctcatgatctc |
| | | | | ccggacccctgaggtcacgtgcgtggtggtggacgtgag |
| | | | | ccacgaagaccccgaggtccagttcaactggtacgtgga |
| | | | | cggcgtggaggtgcataatgccaagacaaagccacggg |
| | | | | aggagcagttcaacagcacgttccgtgtggtcagcgtcct |
| | | | | caccgttgtgcaccaggactggctgaacggcaaggagta |
| | | | | caagtgcaaggtctccaacaaaggcctcccagcccccat |
| | | | | cgagaaaaccatctccaaaaccaaagggcagccccgag |
| | | | | aaccacaggtgtacaccctgcccccatcccgggaggaga |
| | | | | tgaccaagaaccaggtcagcctgacctgcctggtcaaag |
| | | | | gcttctaccccagcgacatcgccgtggagtgggagagca |
| | | | | atgggcagccggagaacaactacaagaccacacctccca |
| | | | | tgctggactccgacggctccttcttcctctacagcaagctca |
| | | | | ccgtggacaagagcaggtggcagcaggggaacgtcttct |
| | | | | catgctccgtgatgcatgaggctctgcacaaccactacac |
| | | | | gcagaagagcctctccctgtctccgggtaaa |
| 532 | | | Human Heavy | ASTKGPSVFPLAPCSRSTSESTAALGC |
| | | | Chain Constant | LVKDYFPEPVTVSWNSGALTSGVHTF |
| | | | Region | PAVLQSSGLYSLSSVVTVPSSSLGTQT |
| | | | (IGHG2*04) | YTCNVDHKPSNTKVDKTVERKCCVE |
| | | | Protein Sequence | CPPCPAPPVAGPSVFLFPPKPKDTLMI |
| | | | | SRTPEVTCVVVDVSHEDPEVQFNWY |
| | | | | VDGVEVHNAKTKPREEQFNSTFRVVS |
| | | | | VLTVVHQDWLNGKEYKCKVSNKGL |
| | | | | PAPIEKTISKTKGQPREPQVYTLPPSRE |
| | | | | EMTKNQVSLTCLVKGFYPSDIAVEWE |
| | | | | SNGQPENNYKTTPPMLDSDGSFFLYS |
| | | | | KLTVDKSRWQQGNVFSCSVMHEALH |
| | | | | NHYTQKSLSLSPGK |
| 533 | Human | IGHG2* | Human Heavy | GCCTCCACCAAGGGCCCATCGGTCT |
| | IgG2 | 06 | Chain Constant | TCCCCCTGGCGCCCTGCTCCAGGAG |
| | constant | | Region | CACCTCCGAGAGCACAGCGGCCCTG |
| | region | | (IGHG2*06) | GGCTGCCTGGTCAAGGACTACTTCC |
| | | | Nucleotide | CCGAACCGGTGACGGTGTCGTGGAA |
| | | | Sequence | CTCAGGCGCTCTGACCAGCGGCGTG |
| | | | | CACACCTTCCCGGCTGTCCTACAGT |
| | | | | CCTCAGGACTCTACTCCCTCAGCAG |
| | | | | CGTGGTGACCGTGCCCTCCAGCAAC |
| | | | | TTCGGCACCCAGACCTACACCTGCA |
| | | | | ACGTAGATCACAAGCCCAGCAACA |
| | | | | CCAAGGTGGACAAGACAGTTGAGC |
| | | | | GCAAATGTTGTGTCGAGTGCCCACC |
| | | | | GTGCCCAGCACCACCTGTGGCAGGA |
| | | | | CCGTCAGTCTTCCTCTTCCCCCCAAA |
| | | | | ACCCAAGGACACCCTCATGATCTCC |
| | | | | CGGACCCCTGAGGTCACGTGCGTGG |
| | | | | TGGTGGACGTGAGCCACGAAGACC |
| | | | | CCGAGGTCCAGTTCAACTGGTACGT |
| | | | | GGACGGCGTGGAGGTGCATAATGC |
| | | | | CAAGACAAAGCCACGGGAGGAGCA |
| | | | | GTTCAACAGCACGTTCCGTGTGGTC |
| | | | | AGCGTCCTCACCGTCGTGCACCAGG |
| | | | | ACTGGCTGAACGGCAAGGAGTACA |
| | | | | AGTGCAAGGTCTCCAACAAAGGCCT |
| | | | | CCCAGCCCCCATCGAGAAAACCATC |
| | | | | TCCAAAACCAAAGGGCAGCCCCGA |
| | | | | GAACCACAGGTGTACACCCTGCCCC |
| | | | | CATCCCGGGAGGAGATGACCAAGA |
| | | | | ACCAGGTCAGCCTGACCTGCCTGGT |
| | | | | CAAAGGCTTCTACCCCAGCGACATC |
| | | | | TCCGTGGAGTGGGAGAGCAATGGG |
| | | | | CAGCCGGAGAACAACTACAAGACC |
| | | | | ACACCTCCCATGCTGGACTCCGACG |
| | | | | GCTCCTTCTTCCTCTACAGCAAGCTC |
| | | | | ACCGTGGACAAGAGCAGGTGGCAG |
| | | | | CAGGGGAACGTCTTCTCATGCTCCG |
| | | | | TGATGCATGAGGCTCTGCACAACCA |
| | | | | CTACACACAGAAGAGCCTCTCCCTG |
| | | | | TCTCCGGGTAAA |
| 534 | | | Human Heavy | ASTKGPSVFPLAPCSRSTSESTAALGC |
| | | | Chain Constant | LVKDYFPEPVTVSWNSGALTSGVHTFP |
| | | | Region | AVLQSSGLYSLSSVVTVPSSNFGTQT |
| | | | (IGHG2*06) | YTCNVDHKPSNTKVDKTVERKCCVEC |
| | | | Protein Sequence | PPCPAPPVAGPSVFLFPPKPKDTLMI |
| | | | | SRTPEVTCVVVDVSHEDPEVQFNWY |
| | | | | VDGVEVHNAKTKPREEQFNSTFRVVS |
| | | | | VLTVVHQDWLNGKEYKCKVSNKGLPA |
| | | | | PIEKTISKTKGQPREPQVYTLPPSRE |
| | | | | EMTKNQVSLTCLVKGFYPSDISVEWE |
| | | | | SNGQPENNYKTTPPMLDSDGSFFLYS |
| | | | | KLTVDKSRWQQGNVFSCSVMHEALH |
| | | | | NHYTQKSLSLSPGK |
| 535 | Human Cλ | IGLC7* | Cλ Light Chain | GGTCAGCCCAAGGCTGCCCCCTCGG |
| | constant | 03 | Constant Region | TCACTCTGTTCCCACCCTCCTCTGAG |
| | region | | (IGLC7*03) | GAGCTTCAAGCCAACAAGGCCACA |
| | | | Nucleotide | CTGGTGTGTCTCGTAAGTGACTTCA |
| | | | Sequence | ACCCGGGAGCCGTGACAGTGGCCTG |
| | | | | GAAGGCAGATGGCAGCCCCGTCAA |
| | | | | GGTGGGAGTGGAGACCACCAAACC |
| | | | | CTCCAAACAAAGCAACAACAAGTA |
| | | | | TGCGGCCAGCAGCTACCTGAGCCTG |
| | | | | ACGCCCGAGCAGTGGAAGTCCCAC |
| | | | | AGAAGCTACAGCTGCCGGGTCACGC |
| | | | | ATGAAGGGAGCACCGTGGAGAAGA |
| | | | | CAGTGGCCCCTGCAGAATGCTCT |
| 536 | | | Cλ Light Chain | GQPKAAPSVTLFPPSSEELQANKATL |
| | | | Constant Region | VCLVSDFNPGAVTVAWKADGSPVKV |
| | | | (IGLC7*03) | GVETTKPSKQSNNKYAASSYLSLTPE |
| | | | Amino Acid | QWKSHRSYSCRVTHEGSTVEKTVAP |
| | | | Sequence | AECS |
| 537 | Human | IGHG1* | WT human IgG1 | gcctccaccaagggcccatcggtcttccccctggcaccct |
| | WT IgG1 | 01 | nucleotide | cctccaagagcacctctgggggcacagcggccctgggct |
| | constant | & | sequence #2 | gcctggtcaaggactacttccccgaaccggtgacggtgtc |
| | region | IGHG1* | | gtggaactcaggcgccctgaccagcggcgtgcacacctt |
| | | 05 | | cccggctgtcctacagtcctcaggactctactccctcagca |
| | | (IgG1) | | gcgtggtgaccgtgccctccagcagcttgggcacccaga |
| | | | | cctacatctgcaacgtgaatcacaagcccagcaacaccaa |
| | | | | ggtggacaagaaagttgagcccaaatcttgtgacaaaact |
| | | | | cacacatgcccaccgtgcccagcacctgaactcctgggg |
| | | | | ggaccgtcagtcttcctcttccccccaaaacccaaggaca |
| | | | | ccctcatgatctcccggacccctgaggtcacatgcgtggt |
| | | | | ggtggacgtgagccacgaagaccctgaggtcaagttcaa |
| | | | | ctggtacgtggacggcgtggaggtgcataatgccaagac |
| | | | | aaagccgcgggaggagcagtacaacagcacgtaccgg |
| | | | | gtggtcagcgtcctcaccgtcctgcaccaggactggctga |
| | | | | atggcaaggagtacaagtgcaaggtctccaacaaagccc |
| | | | | tcccagcccccatcgagaaaaccatctccaaagccaaag |
| | | | | ggcagccccgagaaccacaggtgtacaccctgcccccat |
| | | | | cccgggatgagctgaccaagaaccaggtcagcctgacct |
| | | | | gcctggtcaaaggcttctatcccagcgacatcgccgtgga |
| | | | | gtgggagagcaatgggcagccggagaacaactacaaga |
| | | | | ccacgcctcccgtgctggactccgacggctccttcttcctct |
| | | | | acagcaagctcaccgtggacaagagcaggtggcagcag |
| | | | | gggaacgtcttctcatgctccgtgatgcatgaggctctgca |
| | | | | caaccactacacgcagaagagcctctccctgtctccgggt |
| | | | | aaa |
| 538 | Human Cλ | IGLC2* | Cλ Light Chain | GQPKAAPSVTLFPPSSEELQANKATL |
| | constant | 01 | Constant Region | VCLISDFYPGAVTVAWKADSSPVKA |
| | region | | Amino Acid | GVETTTPSKQSNNKYAASSYLSLTPE |
| | | | Sequence #2- | QWKSHRSYSCQVTHEGSTVEKTVAP |
| | | | Encoded by | TECS |
| | | | nucleotide | |
| | | | sequence version | |
| | | | A & B | |

TABLE S3
1.21.1.13. SEQ ID NOS: 539-562

Sequence

hIgG1 FIT-Ig bispecific 1a

| | | |
|---|---|---|
| Antibody A | anti-ICOS | |
| | STIM003 | |
| Antibody B | anti-PD-L1 | |
| | 84G09 | |
| FIT-Ig | SEQ ID NO: 539 | DIQMTQSPASLSASLGETVTIQCRASEDIYSGLAWFQQKPGKSPQLLIYGA |
| Construct #1 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGECEVQLVESGGGLTQPGKSLKLSCEASGFTFSSFTMHWVRQSP |
| | | GKGLEWVAFIRSGSGIVFYADAVRGRFTISRDNAKNLLFLQMNDLKSEDTA |
| | | MYYCARRPLGHNTFDSWGQGTLVTVSSASTKGPSVFPLAPSSKSTSGGTAA |
| | | LGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| FIT-Ig | SEQ ID NO: 540 | EVQLVESGGGLVQPGRSLKLSCAASGFTFSDFYMAWVRQAPKKGLEWVASI |
| Construct #2 | | SYEGSSTYYGDSVMGRFTISRDNAKSTLYLQMNSLRSEDTATYYCARQREA |
| | | NWEDWGQGVMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKV |
| FIT-Ig | SEQ ID NO: 541 | DIVMTQSPSSLAVSPGEKVTMTCKSSQSLYYSGVKENLLAWYQQKPGQSPK |
| Construct #3 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGEC |

hIgG1 FIT-Ig bispecific 1b

| | | |
|---|---|---|
| Antibody A | anti-PD-L1 | |
| | 84G09 | |
| Antibody B | anti-ICOS | |
| | STIM003 | |
| FIT-Ig | SEQ ID NO: 542 | DIVMTQSPSSLAVSPGEKVTMTCKSSQSLYYSGVKENLLAWYQQKPGQSPK |
| Construct #1 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGECEVQLVESGGGLVQPGRSLKLSCAASGFTFSDFYMA |
| | | WVRQAPKKGLEWVASISYEGSSTYYGDSVMGRFTISRDNAKSTLYLQMNSL |
| | | RSEDTATYYCARQREANWEDWGQGVMVTVSSASTKGPSVFPLAPSSKSTSG |
| | | GTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLSL |
| | | SPGK |
| FIT-Ig | SEQ ID NO: 543 | EVQLVESGGGLTQPGKSLKLSCEASGFTFSSFTMHWVRQSPGKGLEWVAFI |
| Construct #2 | | RSGSGIVFYADAVRGRFTISRDNAKNLLFLQMNDLKSEDTAMYYCARRPLG |
| | | HNTFDSWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNV |
| | | NHKPSNTKVDKKV |
| FIT-Ig | SEQ ID NO: 544 | DIQMTQSPASLSASLGETVTIQCRASEDIYSGLAWFQQKPGKSPQLLIYGA |
| Construct #3 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQKVDNAW |
| | | LQSGNSQESVTEQDSKDSTYSLSSTLTLSKADYEKHKVYACEVTHQGLSSP |
| | | VTKSFNRGEC |

hIgG1 FIT-Ig bispecific 2a

| | | |
|---|---|---|
| Antibody A | anti-ICOS | |
| | STIM001 | |
| Antibody B | anti-PD-L1 | |
| | 1D05 | |
| FIT-Ig | SEQ ID NO: 545 | DIQMTQSPASLSASLGETVTIQCRASEDIYSGLAWFQQKPGKSPQLLIYGA |
| Construct #1 | | SSLQDGVPSRFSGSGSGTQYSLKISSMQTEDEGVYFCQQGLKYPPTFGSGT |
| | | KLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSP |
| | | IVKSFNRNECEVQLVESGGGLTQPGKSLKLSCEASGFTFSSFTMHWVRQSP |
| | | GKGLEWVAFIRSGSGIVFYADAVRGRFTISRDNAKNLLFLQMNDLKSEDTA |
| | | MYYCARRPLGHNTFDSWGQGTLVTVSSAKTTAPSVYPLAPVCGDTTGSSVT |
| | | LGCLVKGYFPEPVTLTWNSGSLSSGVHTFPAVLQSDLYTLSSSVTVTSSTW |
| | | PSQSITCNVAHPASSTKVDKKIEPRGPTIKPCPPCKCPAPNLLGGPSVFIF |
| | | PPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRA |
| | | PQVYVLPPPEEEMTKKQVTLTCMVTDFMPEDIYVEWTNNGKTELNYKNTEP |
| | | VLDSDGSYFMYSKLRVEKKNWVERNSYSCSVVHEGLHNHHTTKSFSRTPGK |
| FIT-Ig | SEQ ID NO: 546 | EVQLVESGGGLVQPGRSLKLSCAASGFTFSDFYMAWVRQAPKKGLEWVASI |
| Construct #2 | | SYEGSSTYYGDSVMGRFTISRDNAKSTLYLQMNSLRSEDTATYYCARQREA |
| | | NWEDWGQGVMVTVSSAKTTAPSVYPLAPVCGDTTGSSVTLGCLVKGYFPEP |
| | | VTLTWNSGSLSSGVHTFPAVLQSDLYTLSSSVTVTSSTWPSQSITCNVAHP |
| | | ASSTKVDKKI |
| FIT-Ig | SEQ ID NO: 547 | DIVMTQSPSSLAVSPGEKVTMTCKSSQSLYYSGVKENLLAWYQQKPGQSPK |
| Construct #3 | | LLIYYASIRFTGVPDRFTGSGSGTDYTLTITSVQAEDMGQYFCQQGINNPL |
| | | TFGDGTKLEIKRTDAAPTVSIFPPSSEQLTSGGASVVCFLNNFYPKDINVK |
| | | WKIDGSERQNGVLNSWTDQDSKDSTYSMSSTLTLTKDEYERHNSYTCEATH |
| | | KTSTSPIVKSFNRNEC |

hIgG1 FIT-Ig bispecific 2b

| | | |
|---|---|---|
| Antibody A | anti-PD-L1 | |
| | 1D05 | |
| Antibody B | anti-ICOS | |
| | STIM001 | |
| FIT-Ig | SEQ ID NO: 548 | DIVMTQSPSSLAVSPGEKVTMTCKSSQSLYYSGVKENLLAWYQQKPGQSPK |
| Construct #1 | | LLIYYASIRFTGVPDRFTGSGSGTDYTLTITSVQAEDMGQYFCQQGINNPL |
| | | TFGDGTKLEIKRTDAAPTVSIFPPSSEQLTSGGASVVCFLNNFYPKDINVK |
| | | WKIDGSERQNGVLNSWTDQDSKDSTYSMSSTLTLTKDEYERHNSYTCEATH |
| | | KTSTSPIVKSFNRNECEVQLVESGGGLVQPGRSLKLSCAASGFTFSDFYMA |
| | | WVRQAPKKGLEWVASISYEGSSTYYGDSVMGRFTISRDNAKSTLYLQMNSL |
| | | RSEDTATYYCARQREANWEDWGQGVMVTVSSAKTTAPSVYPLAPVCGDTTG |
| | | SSVTLGCLVKGYFPEPVTLTWNSGSLSSGVHTFPAVLQSDLYTLSSSVTVT |
| | | SSTWPSQSITCNVAHPASSTKVDKKIEPRGPTIKPCPPCKCPAPNLLGGPS |
| | | VFIFPPKIKDVLMISLSPIVTCVVVDVSEDDPDVQISWFVNNVEVHTAQTQ |
| | | THREDYNSTLRVVSALPIQHQDWMSGKEFKCKVNNKDLPAPIERTISKPKG |
| | | SVRAPQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTKSFSR |
| | | TPGK |
| FIT-Ig | SEQ ID NO: 549 | EVQLVESGGGLTQPGKSLKLSCEASGFTFSSFTMHWVRQSPGKGLEWVAFI |
| Construct #2 | | RSGSGIVFYADAVRGRFTISRDNAKNLLFLQMNDLKSEDTAMYYCARRPLG |
| | | HNTFDSWGQGTLVTVSSAKTTAPSVYPLAPVCGDTTGSSVTLGCLVKGYFP |
| | | EPVTLTWNSGSLSSGVHTFPAVLQSDLYTLSSSVTVTSSTWPSQSITCNVA |
| | | HPASSTKVDKKI |
| FIT-Ig | SEQ ID NO: 550 | DIQMTQSPASLSASLGETVTIQCRASEDIYSGLAWFQQKPGKSPQLLIYGA |
| Construct #3 | | SSLQDGVPSRFSGSGSGTQYSLKISSMQTEDEGVYFCQQGLKYPPTFGSGT |
| | | KLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSP |
| | | IVKSFNRNEC |

hIgG1 FIT-Ig bispecific 3a

| | | |
|---|---|---|
| Antibody A | anti-ICOS | |
| | STIM003 | |
| Antibody B | anti-PD-L1 | |
| | 1D05 | |
| FIT-Ig | SEQ ID NO: 551 | DIQMTQSPASLSASLGETVTIQCRASEDIYSGLAWFQQKPGKSPQLLIYGA |
| Construct #1 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGECEVQLVESGGGLTQPGKSLKLSCEASGFTFSSFTMHWVRQSP |
| | | GKGLEWVAFIRSGSGIVFYADAVRGRFTISRDNAKNLLFLQMNDLKSEDTA |
| | | MYYCARRPLGHNTFDSWGQGTLVTVSSASTKGPSVFPLAPSSKSTSGGTAA |
| | | LGCLVKDYFPEPVTVSWNSGALTSGVHTFPAVLQSSGLYSLSSVVTVPSSS |
| | | LGTQTYICNVNHKPSNTKVDKKVEPKSCDKTHTCPPNLLGGPSVFIFPPKI |
| | | KDVLMISLSPIVTCVVVDVSEDDPDVQISWFVNNVEVHTAQTQTHREDYNS |
| | | TLRVVSALPIQHQDWMSGKEFKCKVNNKDLPAPIERTISKPKGSVRAPQVY |
| | | VLPPPEEEMTKKQVTLTCMVTDFMPEDIYVEWTNNGKTELNYKNTEPVLDS |
| | | DGSYFMYSKLRVEKKNWVERNSYSCSVVHEGLHNHHTTKSFSRTPGK |
| FIT-Ig | SEQ ID NO: 552 | EVQLVESGGGLVQPGRSLKLSCAASGFTFSDFYMAWVRQAPKKGLEWVASI |
| Construct #2 | | SYEGSSTYYGDSVMGRFTISRDNAKSTLYLQMNSLRSEDTATYYCARQREA |
| | | NWEDWGQGVMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKV |
| FIT-Ig | SEQ ID NO: 553 | DIVMTQSPSSLAVSPGEKVTMTCKSSQSLYYSGVKENLLAWYQQKPGQSPK |
| Construct #3 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGEC |

hIgG1 FIT-Ig bispecific 3b

| | | |
|---|---|---|
| Antibody A | anti-PD-L1 | |
| | 1D05 | |
| Antibody B | anti-ICOS | |
| | STIM003 | |
| FIT-Ig | SEQ ID NO: 554 | DIVMTQSPSSLAVSPGEKVTMTCKSSQSLYYSGVKENLLAWYQQKPGQSPK |
| Construct #1 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGECEVQLVESGGGLVQPGRSLKLSCAASGFTFSDFYMA |
| | | WVRQAPKKGLEWVASISYEGSSTYYGDSVMGRFTISRDNAKSTLYLQMNSL |
| | | RSEDTATYYCARQREANWEDWGQGVMVTVSSASTKGPSVFPLAPSSKSTSG |
| | | GTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPNLLGGPSVFIF |
| | | PPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRA |
| | | PQVYVLPPPEEEMTKKQVTLTCMVTDFMPEDIYVEWTNNGKTELNYKNTEP |
| | | VLDSDGSYFMYSKLRVEKKNWVERNSYSCSVVHEGLHNHHTTKSFSRTPGK |
| FIT-Ig | SEQ ID NO: 555 | EVQLVESGGGLTQPGKSLKLSCEASGFTFSSFTMHWVRQSPGKGLEWVAFI |
| Construct #2 | | RSGSGIVFYADAVRGRFTISRDNAKNLLFLQMNDLKSEDTAMYYCARRPLG |
| | | HNTFDSWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNV |
| | | NHKPSNTKVDKKV |
| FIT-Ig | SEQ ID NO: 556 | DIQMTQSPASLSASLGETVTIQCRASEDIYSGLAWFQQKPGKSPQLLIYGA |
| Construct #3 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |

hIgG1 FIT-Ig bispecific 4a

| | | |
|---|---|---|
| Antibody A | anti-ICOS | |
| | STIM001 | |
| Antibody B | anti-PD-L1 | |
| | 84G09 | |
| FIT-Ig | SEQ ID NO: 557 | DIQMTQSPASLSASLGETVTIQCRASEDIYSGLAWFQQKPGKSPQLLIYGA |
| Construct #1 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGECEVQLVESGGGLTQPGKSLKLSCEASGFTFSSFTMHWVRQSP |
| | | GKGLEWVAFIRSGSGIVFYADAVRGRFTISRDNAKNLLFLQMNDLKSEDTA |
| | | MYYCARRPLGHNTFDSWGQGTLVTVSSASTKGPSVFPLAPSSKSTSGGTAA |
| | | LGCLVKDYFPEPVTVSWNSGALTSGVHTFPAVLQSSGLYSLSSVVTVPSSS |
| | | LGTQTYICNVNHKPSNTKVDKKVEPKSCDKTHTCPPNLLGGPSVFIFPPKI |
| | | KDVLMISLSPIVTCVVVDVSEDDPDVQISWFVNNVEVHTAQTQTHREDYNS |
| | | TLRVVSALPIQHQDWMSGKEFKCKVNNKDLPAPIERTISKPKGSVRAPQVY |
| | | VLPPPEEEMTKKQVTLTCMVTDFMPEDIYVEWTNNGKTELNYKNTEPVLDS |
| | | DGSYFMYSKLRVEKKNWVERNSYSCSVVHEGLHNHHTTKSFSRTPGK |
| FIT-Ig | SEQ ID NO: 558 | EVQLVESGGGLVQPGRSLKLSCAASGFTFSDFYMAWVRQAPKKGLEWVASI |
| Construct #2 | | SYEGSSTYYGDSVMGRFTISRDNAKSTLYLQMNSLRSEDTATYYCARQREA |
| | | NWEDWGQGVMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNH |
| | | KPSNTKVDKKV |
| FIT-Ig | SEQ ID NO: 559 | DIVMTQSPSSLAVSPGEKVTMTCKSSQSLYYSGVKENLLAWYQQKPGQSPK |
| Construct #3 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGEC |

hIgG1 FIT-Ig bispecific 4b

| | | |
|---|---|---|
| Antibody A | anti-PD-L1 | |
| | 84G09 | |
| Antibody B | anti-ICOS | |
| | STIM001 | |
| FIT-Ig | SEQ ID NO: 560 | DIVMTQSPSSLAVSPGEKVTMTCKSSQSLYYSGVKENLLAWYQQKPGQSPK |
| Construct #1 | | LLIYYASIRFTGVPDRFTGSGSGTDYTLTITSVQAEDMGQYFCQQGINNPL |
| | | TFGDGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTH |
| | | QGLSSPVTKSFNRGECEVQLVESGGGLVQPGRSLKLSCAASGFTFSDFYMA |
| | | WVRQAPKKGLEWVASISYEGSSTYYGDSVMGRFTISRDNAKSTLYLQMNSL |
| | | RSEDTATYYCARQREANWEDWGQGVMVTVSSASTKGPSVFPLAPSSKSTSG |
| | | GTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPNLLGGPSVFIF |
| | | PPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRA |
| | | PQVYVLPPPEEEMTKKQVTLTCMVTDFMPEDIYVEWTNNGKTELNYKNTEP |
| | | VLDSDGSYFMYSKLRVEKKNWVERNSYSCSVVHEGLHNHHTTKSFSRTPGK |
| FIT-Ig | SEQ ID NO: 561 | EVQLVESGGGLTQPGKSLKLSCEASGFTFSSFTMHWVRQSPGKGLEWVAFI |
| Construct #2 | | RSGSGIVFYADAVRGRFTISRDNAKNLLFLQMNDLKSEDTAMYYCARRPLG |
| | | HNTFDSWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNV |
| | | NHKPSNTKVDKKV |
| FIT-Ig | SEQ ID NO: 562 | DIQMTQSPASLSASLGETVTIQCRASEDIYSGLAWFQQKPGKSPQLLIYGA |
| Construct #3 | | SSLQDGVPSRFSGSGSGTQYSLKISSMQTEDEGVYFCQQGLKYPPTFGSGT |
| | | KLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |

TABLE S4
1.21.1.14. Sequences of antibody heavy chain variable regions obtained from additional clones
CDRs are defined according to IMGT.

| CLONE_ID | VH_NUCLEOTIDE_SEQUENCE | VH_AMINO_ACID_SEQ | HCDR1 | HCDR2 | HCDR3 |
|---|---|---|---|---|---|
| CL-61091 | CAGGTTCAACTGATGCAGTCTGGAACTGAGGTGAAGAAG | QVQLMQSGTEVKKPGAS | GYTFTTY | ISAYSGD | ARSSGWP |
| | CCTGGGGCCTCAGTGAAGGTCTCCTGCAAGACTTCTGGT | VKVSCKTSGYTFTTYGI | G | T | HHYGMDV |
| | TACACCTTTACCACCTATGGTATCACTTGGGTGCGACAG | TWVRQAPGQGLEWMGWI | SEQ ID | SEQ ID | SEQ ID |
| | GCCCCTGGACAAGGGCTTGAGTGGATGGGATGGATCAGC | SAYSGDTDYAQKFQGRV | NO: 565 | NO: 566 | NO: 567 |
| | GCTTACAGTGGTGACACAGACTATGCACAGAAGTTCCAG | TVTTDTSTNTAYMELRS | | | |
| | GGCAGAGTCACCGTGACAACAGACACATCCACGAACACA | LKSDDTAVYYCARSSGW | | | |
| | GCCTACATGGAGTTGAGGAGCCTGAAATCTGACGACACG | PHHYGMDVWGQGTTVTV | | | |
| | GCCGTGTATTATTGTGCGAGAAGTAGTGGCTGGCCCCAC | SS | | | |
| | CACTACGGTATGGACGTCTGGGGCCAAGGGACCACGGTC | SEQ ID NO: 564 | | | |
| | ACCGTCTCCTCAG | | | | |
| | SEQ ID NO: 563 | | | | |
| CL-64536 | CAGGTTCAACTGGTGCAGTCTGGAGGTGAGGTGAAAAAG | QVQLVQSGGEVKKPGAS | GYTFTSY | ISAYNGN | ARSTSYY |
| | CCTGGGGCCTCAGTGAAGGTCTCCTGCAAGGCTTCTGGT | VKVSCKASGYTFTSYGF | G | T | GSGTLYG |
| | TACACCTTTACCAGCTATGGTTTCAGCTGGGTGCGACAG | SWVRQAPGQGLEWMGWI | SEQ ID | SEQ ID | MDV |
| | GCCCCTGGACAAGGACTAGAGTGGATGGGATGGATCAGC | SAYNGNTNYAQKLQGRV | NO: 377 | NO: 378 | SEQ ID |
| | GCTTACAATGGTAACACAAACTATGCACAGAAGCTCCAG | SMTTDTSTSTAYMELRS | | | NO: 570 |
| | GGCAGAGTCTCCATGACCACAGACACATCCACGAGCACA | LRSDDTAVYFCARSTSY | | | |
| | GCCTACATGGAGCTGAGGAGCTTGAGATCTGACGACACG | YGSGTLYGMDVWGQGTT | | | |
| | GCCGTGTATTTCTGTGCGCGATCTACGTCTTACTATGGT | VTVSS | | | |
| | TCGGGGACCCTATACGGTATGGACGTCTGGGGCCAAGGG | SEQ ID NO: 569 | | | |
| | ACCACGGTCACCGTCTCCTCAG | | | | |
| | SEQ ID NO: 568 | | | | |
| CL-64837 | CAGGTTCAACTGGTGCAGTCTGGAGGTGAGGTGAAGAAG | QVQLVQSGGEVKKPGAS | GYTFTSY | ISAYNGN | ARSTSYY |
| | CCTGGGGCCTCAGTGAAGGTCTCCTGCAAGGCTTCTGGT | VKVSCKASGYTFTSYGF | G | T | GSGTLYG |
| | TACACCTTTACCAGCTATGGTTTCAGCTGGGTGCGACAG | SWVRQAPGQGLEWMGWI | SEQ ID | SEQ ID | MDV |
| | GCCCCTGGACAAGGACTAGAGTGGATGGGATGGATCAGC | SAYNGNTNYAQKLQGRV | NO: 377 | NO: 378 | SEQ ID |
| | GCTTACAATGGTAACACAAACTATGCACAGAAGCTCCAG | SMTTDTSTSTAYMELRS | | | NO: 570 |
| | GGCAGAGTCTCCATGACCACAGACACATCCACGAGCACA | LRSDDTAVYYCARSTSY | | | |
| | GCCTACATGGAGCTGAGGAGCTTGAGATCTGACGACACG | YGSGTLYGMDVWGQGTT | | | |
| | GCCGTGTATTACTGTGCGCGATCTACGTCTTACTATGGT | VTVSS | | | |
| | TCGGGGACCCTCTACGGTATGGACGTCTGGGGCCAAGGG | SEQ ID NO: 572 | | | |
| | ACCACGGTCACCGTCTCCTCAG | | | | |
| | SEQ ID NO: 571 | | | | |
| CL-64841 | CAGGTTCAACTGGTGCAGTCTGGAGGTGAGGTGAAAAAG | QVQLVQSGGEVKKPGAS | GYTFTSY | ISAYNGN | ARSTSYY |
| | CCTGGGGCCTCAGTGAAGGTCTCCTGCAAGGCTTCTGGT | VKVSCKASGYTFTSYGF | G | T | GSGTLYG |
| | TACACCTTTACCAGCTATGGTTTCAGCTGGGTGCGACAG | SWVRQAPGQGLEWMGWI | SEQ ID | SEQ ID | MDV |
| | GCCCCTGGACAAGGACTAGAGTGGATGGGATGGATCAGC | SAYNGNTNYAQKLQGRV | NO: 377 | NO: 378 | SEQ ID |
| | GCTTACAATGGTAACACAAACTATGCACAGAAGCTCCAG | SMTTDTSTSTAYMELRS | | | NO: 570 |
| | GGCAGAGTCTCCATGACCACAGACACATCCACGAGCACA | LRSDDTAVYFCARSTSY | | | |
| | GCCTACATGGAGCTGAGGAGCTTGAGATCTGACGACACG | YGSGTLYGMDVWGQGTT | | | |
| | GCCGTGTATTTCTGTGCGCGATCTACGTCTTACTATGGT | VTVSS | | | |
| | TCGGGGACCCTATACGGTATGGACGTCTGGGGCCAAGGG | SEQ ID NO: 574 | | | |
| | ACCACGGTCACCGTCTCCTCAG | | | | |
| | SEQ ID NO: 573 | | | | |
| CL-64912 | CAGGTTCAACTGGTGCAGTCTGGAGGTGAGGTGAAAAAG | QVQLVQSGGEVKKPRAS | GYTFTSY | ISGYNGN | ARSTSYY |
| | CCTCGGGCCTCAGTGAAGGTCTCCTGCAAGGCTTCTGGT | VKVSCKASGYTFTSYVF | V | T | GAGTLYG |
| | TACACCTTTACCAGCTATGTGTTCAGCTGGGTGCGACAT | SWVRHAAGQGLEWMGWI | SEQ ID | SEQ ID | MDV |
| | GCCGCTGGACAAGGACTAGAGTGGATGGGATGGATCAGC | SGYNGNTNYAQKLQCGV | NO: 577 | NO: 578 | SEQ ID |
| | GGTTACAATGGTAACACAAACTATGCACAGAAGCTCCAG | SMTADTSTSTAYMELRS | | | NO: 579 |
| | TGCGGAGTCTCGATGACCGCAGACACATCCACGAGCACA | LRSDDTAVYFCARSTSY | | | |
| | GCCTACATGGAGCTGAGGAGCTTGAGATCTGACGACACG | YGAGTLYGMDVWGQGTT | | | |
| | GCCGTGTATTTCTGTGCGCGATCTACGTCTTACTATGGT | VTVSS | | | |
| | GCGGGGACCCTATACGGTATGGACGTCTGGGGCCAAGGG | SEQ ID NO: 576 | | | |
| | ACCACGGTCACCGTCTCCTCAG | | | | |
| | SEQ ID NO: 575 | | | | |
| CL-71642 | GAGGTGCAGCTGGTGGAGTCTGGGGGAGGTGTGGTACGG | EVQLVESGGGVVRPGGS | GFTFDDY | INWNGGS | AADYYGS |
| | CCTGGGGGGTCCCTGAGACTCTCCTGTGCAGCCTCTGGA | LRLSCAASGFTFDDYGM | G | T | GSYYNVP |
| | TTCACCTTTGATGATTATGGCATGAGCTGGGTCCGCCAA | SWVRQAPGKGLEWVSGI | SEQ ID | SEQ ID | FDY |
| | GCTCCAGGGAAGGGGCTGGAGTGGGTCTCTGGTATTAAT | NWNGGSTGYADSVKGRF | NO: 582 | NO: 583 | SEQ ID |
| | TGGAATGGTGGTAGCACAGGTTATGCAGACTCTGTGAAG | TISRDNAKNSLYLQMNS | | | NO: 584 |
| | GGCCGATTCACCATCTCCAGAGACAACGCCAAGAACTCC | LRAEDTALYYCAADYYG | | | |
| | CTGTATCTGCAAATGAACAGTCTGAGAGCCGAGGACACG | SGSYYNVPFDYWGQGTL | | | |
| | GCCTTGTATTACTGTGCGGCCGATTACTATGGTTCGGGG | VTVSS | | | |
| | AGTTATTATAACGTCCCCTTTGACTACTGGGGCCAGGGA | SEQ ID NO: 581 | | | |
| | ACCCTGGTCACCGTCTCCTCAG | | | | |
| | SEQ ID NO: 580 | | | | |
| CL-74570 | GAGGTGCAGCTGGTGGAGTCTGGGGGAGGTGTGATACGG | EVQLVESGGGVIRPGGS | GFTFDDY | INWIGDN | ARDYFGS |
| | CCTGGGGGGTCCCTGAGACTCTCCTGTGCAGCCTCTGGA | LRLSCAASGFTFDDYGM | G | T | GSYYNVP |
| | TTCACCTTTGATGATTATGGCATGAGCTGGGTCCGCCAA | SWVRQAPGKGLEWVSGI | SEQ ID | SEQ ID | FDY |
| | GCTCCAGGGAAGGGGCTGGAGTGGGTCTCTGGTATTAAT | NWIGDNTDYADSVKGRF | NO: 582 | NO: 587 | SEQ ID |
| | TGGATTGGTGATAACACAGATTATGCAGACTCTGTGAAG | TISRDNAKNSLYLQMNS | | | NO: 588 |
| | GGCCGATTCACCATCTCCAGAGACAACGCCAAGAACTCC | LRAEDTALYYCARDYFG | | | |
| | CTATATCTGCAAATGAACAGTCTGAGAGCCGAGGACACG | SGSYYNVPFDYWGQGTL | | | |
| | GCCTTGTATTACTGTGCGAGAGATTACTTTGGTTCGGGG | VTVSS | | | |
| | AGTTATTATAACGTTCCCTTTGACTACTGGGGCCAGGGA | SEQ ID NO: 586 | | | |
| | ACCCTGGTCACCGTCTCCTCAG | | | | |
| | SEQ ID NO: 585 | | | | |

TABLE S5
1.21.1.15. Sequences of antibody light chain variable regions obtained from additional
clones N terminal E and 5′ nucleotide additions in CL-71642 are shown in bold. These
were not recovered in sequencing but were determined to be present in the sequence by
comparison against the related clones as shown in FIG. 6. CDRs are defined according
to IMGT.

| CLONE_ID | VL_NUCLEOTIDE_SEQUENCE | VL_AMINO_ACID_SEQ | LCDR1 | LCDR2 | LCDR3 |
|---|---|---|---|---|---|
| CL-61091 | GATATTGTGATGACTCAGTCTCCACTCTCCCT | DIVMTQSPLSLPVTPGE | QSLLHSN | LVS | MQALQTP |
| | GCCCGTCACCCCTGGAGAGCCGGCCTCCATCT | PASISCRSSQSLLHSNG | GFNY | SEQ ID | LT |
| | CCTGCAGGTCTAGTCAGAGCCTCCTGCATAGT | FNYFDWYLQKPGQSPQL | SEQ ID | NO: 592 | SEQ ID |
| | AATGGATTCAACTATTTCGATTGGTACCTGCA | LIFLVSNRASGVPDRFS | NO: 591 | | NO: 593 |
| | GAAGCCAGGACAGTCTCCACAGCTCCTGATCT | GSGSGTDFTLKISRVEA | | | |
| | TTTTGGTTTCTAATCGGGCCTCCGGGGTCCCT | EDVGIYYCMQALQTPLT | | | |
| | GACAGGTTCAGTGGCAGTGGATCAGGCACAGA | FGGGTKVEIK | | | |
| | TTTTACACTGAAAATCAGCAGAGTGGAGGCTG | SEQ ID NO: 590 | | | |
| | AGGATGTTGGGATTTATTACTGCATGCAAGCT | | | | |
| | CTACAAACTCCGCTCACTTTCGGCGGAGGGAC | | | | |
| | CAAGGTGGAGATCAAAC | | | | |
| | SEQ ID NO: 589 | | | | |
| CL-64536 | GATATTGTGATGACTCAGTCTCCACTCTCCCT | DIVMTQSPLSLPVTPGE | QSLLHSN | LGS | MQALQTP |
| | GCCCGTCACCCCTGGAGAGCCGGCCTCCATCT | PASISCRSSQSLLHSNG | GYNC | SEQ ID | CS |
| | CCTGCAGGTCTAGTCAGAGCCTCCTGCATAGT | YNCLDWYLQKPGQSPQL | SEQ ID | NO: 371 | SEQ ID |
| | AATGGATACAACTGTTTGGATTGGTACCTGCA | LIYLGSTRASGFPDRFS | NO: 596 | | NO: 400 |
| | GAAGCCAGGGCAGTCTCCACAGCTCCTGATCT | GSGSGTDFTLKISRVEA | | | |
| | ATTTGGGTTCTACTCGGGCCTCCGGGTTCCCT | EDVGVYYCMQALQTPCS | | | |
| | GACAGGTTCAGTGGCAGTGGATCAGGCACAGA | FGQGTKLEIK | | | |
| | TTTTACACTGAAAATCAGCAGAGTGGAGGCTG | SEQ ID NO: 595 | | | |
| | AGGATGTTGGGGTTTATTACTGCATGCAAGCT | | | | |
| | CTACAAACTCCGTGCAGTTTTGGCCAGGGGAC | | | | |
| | CAAGCTGGAGATCAAAC | | | | |
| | SEQ ID NO: 594 | | | | |
| CL-64837 | GATATTGTGATGACTCAGTCTCCACTCTCCCT | DIVMTQSPLSLPVTPGE | QSLLHSN | LGS | MQALQTP |
| | GCCCGTCACCCCTGGAGAGCCGGCCTCCATCT | PASISCRSSQSLLHSNG | GYNC | SEQ ID | CS |
| | CCTGCAGGTCTAGTCAGAGCCTCCTGCATAGT | YNCLDWYLQKPGQSPQL | SEQ ID | NO: 371 | SEQ ID |
| | AATGGATACAACTGTTTGGATTGGTACCTGCA | LIYLGSTRASGFPDRFS | NO: 596 | | NO: 400 |
| | GAAGCCAGGGCAGTCTCCACAGCTCCTGATCT | GSGSGTDFTLKISRVEA | | | |
| | ATTTGGGTTCTACTCGGGCCTCCGGGTTCCCT | EDVGVYYCMQALQTPCS | | | |
| | GACAGGTTCAGTGGCAGTGGATCAGGCACAGA | FGQGTKLEIK | | | |
| | TTTTACACTGAAAATCAGCAGAGTGGAGGCTG | SEQ ID NO: 598 | | | |
| | AGGATGTTGGGGTTTATTACTGCATGCAAGCT | | | | |
| | CTACAAACTCCGTGCAGTTTTGGCCAGGGGAC | | | | |
| | CAAGCTGGAGATCAAAC | | | | |
| | SEQ ID NO: 597 | | | | |
| CL-64841 | GATATTGTGATGACTCAGTCTCCACTCTCCCT | DIVMTQSPLSLPVTPGE | QSLLHSN | LGS | MQALQTP |
| | GCCCGTCACCCCTGGAGAGCCGGCCTCCATCT | PASISCRSSQSLLHSNG | GYNC | SEQ ID | CS |
| | CCTGCAGGTCTAGTCAGAGCCTCCTGCATAGT | YNCLDWYLQKPGQSPQL | SEQ ID | NO: 371 | SEQ ID |
| | AATGGATACAACTGTTTGGATTGGTACCTGCA | LIYLGSTRASGFPDRFS | NO: 596 | | NO: 400 |
| | GAAGCCAGGGCAGTCTCCACAGCTCCTGATCT | GSGSGTDSTLKISRVEA | | | |
| | ATTTGGGTTCTACTCGGGCCTCCGGGTTCCCT | EDVGVYYCMQALQTPCS | | | |
| | GACAGGTTCAGTGGCAGTGGATCAGGCACAGA | FGQGTKLEIK | | | |
| | TTCTACACTGAAAATCAGCAGAGTGGAGGCTG | SEQ ID NO: 600 | | | |
| | AGGATGTTGGGGTTTATTACTGCATGCAAGCT | | | | |
| | CTACAAACTCCGTGCAGTTTTGGCCAGGGGAC | | | | |
| | CAAGCTGGAGATCAAAC | | | | |
| | SEQ ID NO: 599 | | | | |
| CL-64912 | GATATTGTGATGACTCAGTCTCCACTCTCCCT | DIVMTQSPLSLPVTPGE | QSLLHSN | LGS | MQALQTP |
| | GCCCGTCACCCCTGGAGAGCCGGCCTCCATCT | PASISCRSSQSLLHSNG | GYNC | SEQ ID | CS |
| | CCTGCAGGTCTAGTCAGAGCCTCCTGCATAGT | YNCLDWYLQKPGQSPQL | SEQ ID | NO: 371 | SEQ ID |
| | AATGGATACAACTGTTTGGATTGGTACCTGCA | LIYLGSTRASGFPDRFS | NO: 596 | | NO: 400 |
| | GAAGCCAGGGCAGTCTCCACAGCTCCTGATCT | GSGSGTDFTLKISRVEA | | | |
| | ATTTGGGTTCTACTCGGGCCTCCGGGTTCCCT | EDVGVYYCMQALQTPCS | | | |
| | GACAGGTTCAGTGGCAGTGGATCAGGCACAGA | FGQGTKLEIK | | | |
| | TTTTACACTGAAAATCAGCAGAGTGGAGGCTG | SEQ ID NO: 602 | | | |
| | AGGATGTTGGGGTTTATTACTGCATGCAAGCT | | | | |
| | CTACAAACTCCGTGCAGTTTTGGCCAGGGGAC | | | | |
| | CAAGCTGGAGATCAAAC | | | | |
| | SEQ ID NO: 601 | | | | |
| CL-71642 | GAAATTGTGTTGACGCAGTCTCCAGGCACCCT | EIVLTQSPGTLSLSPGE | QSVSSSY | GAS | QQYGSSP |
| | GTCTTTGTCTCCAGGGGAAAGAGCCACCCTCT | RATLSCRASQSVSSSYL | SEQ ID | SEQ ID | FT |
| | CCTGCAGGGCCAGTCAGAGTGTTAGCAGCAGC | AWYQQKPGQAPRLLIYG | NO: 426 | NO: 413 | SEQ ID |
| | TACTTAGCCTGGTACCAGCAGAAACCTGGCCA | ASSRATGIPDRFSGSGS | | | NO: 605 |
| | GGCTCCCAGGCTCCTCATCTATGGTGCATCCA | GTDFTLTISRLEPEDFA | | | |
| | GCAGGGCCACTGGCATCCCAGACAGGTTCAGT | VYYCQQYGSSPFTFGPG | | | |
| | GGCAGTGGGTCTGGGACAGACTTCACTCTCAC | TKVDIK | | | |
| | CATCAGCAGACTGGAGCCTGAAGATTTTGCAG | SEQ ID NO: 604 | | | |
| | TGTATTACTGTCAGCAGTATGGTAGCTCACCT | | | | |
| | TTCACTTTCGGCCCTGGGACCAAAGTGGATAT | | | | |
| | CAAAC | | | | |
| | SEQ ID NO: 603 | | | | |
| CL-74570 | GAAATTGTGTTGACGCAGTCTCCAGGCACCCT | EIVLTQSPGTLSLSPGE | QSVSSSY | GAS | HQYGNSP |
| | GTCTTTGTCTCCAGGGGAAAGAGCCACCCTCT | RATLSCRASQSVSSSYL | SEQ ID | SEQ ID | FT |
| | CCTGCAGGGCCAGTCAGAGTGTTAGCAGCAGC | AWYQQKPGQAPRLLIYG | NO: 426 | NO: 413 | SEQ ID |
| | TACTTAGCCTGGTACCAGCAGAAACCTGGCCA | ASSRATGIPDRFSGSGS | | | NO: 608 |
| | GGCTCCCAGGCTCCTCATCTATGGTGCATCCA | GTDFTLTISRLEPEDFA | | | |
| | GCAGGGCCACTGGCATCCCAGACAGGTTCAGT | VYYCHQYGNSPFTFGPG | | | |
| | GGCAGTGGGTCTGGGACAGACTTCACTCTCAC | TKVDIK | | | |
| | CATCAGCAGACTGGAACCTGAAGATTTTGCAG | SEQ ID NO: 607 | | | |
| | TATATTACTGTCACCAGTATGGTAATTCACCA | | | | |
| | TTCACTTTCGGCCCTGGGACCAAAGTGGATAT | | | | |
| | CAAAC | | | | |
| | SEQ ID NO: 606 | | | | |

1. A method of treating a disease or condition amenable to therapy by depleting regulatory T cells (Tregs) and/or increasing effector T cell (Teff) response in a subject in need thereof, the method comprising administering to the subject an anti-ICOS antibody or antigen-binding fragment thereof that binds the extracellular domain of human and/or mouse ICOS, wherein the anti-ICOS antibody or antigen-binding fragment thereof is administered to the subject at a dose of about 0.8 mg to 10 mg.

2. The method of claim 1, wherein the anti-ICOS antibody or antigen-binding fragment thereof comprises heavy chain complimentary determining regions (HCDRs) HCDR1, HCDR2, and HCDR3, and light chain complimentary determining regions (LCDRs) LCDR1, LCDR2, and LCDR3, wherein:
(a) HCDR1, HCDR2, and HCDR3 comprise sequences having at least 85%, 90%, or 95% sequence identity to the amino acid sequences SEQ ID NO: 363, SEQ ID NO: 364, and SEQ ID NO: 365 and LCDR1, LCDR2, and LCDR3 comprise sequences having at least 85%, 90%, or 95% sequence identity to the amino acid sequences SEQ ID NO: 370, SEQ ID NO: 371, SEQ ID NO: 372;
(b) HCDR1, HCDR2, and HCDR3 comprise the sequences having at least 85%, 90%, or 95% sequence identity to the amino acid sequences SEQ ID NO: 377, SEQ ID NO: 378, and SEQ ID NO: 379 and LCDR1, LCDR2, and LCDR3 comprise the sequences having at least 85%, 90%, or 95% sequence identity to the amino acid sequences SEQ ID NO: 384, SEQ ID NO: 385, SEQ ID NO: 386;
(c) HCDR1, HCDR2, and HCDR3 comprise the sequences having at least 85%, 90%, or 95% sequence identity to the amino acid sequences SEQ ID NO: 391, SEQ ID NO: 392, and SEQ ID NO: 393 and LCDR1, LCDR2, and LCDR3 comprise the sequences having at least 85%, 90%, or 95% sequence identity to the amino acid sequences SEQ ID NO: 398, SEQ ID NO: 399, SEQ ID NO: 400;
(d) HCDR1, HCDR2, and HCDR3 comprise the sequences having at least 85%, 90%, or 95% sequence identity to the amino acid sequences SEQ ID NO: 405, SEQ ID NO: 406, and SEQ ID NO: 407 and LCDR1, LCDR2, and LCDR3 comprise the sequences having at least 85%, 90%, or 95% sequence identity to the amino acid sequences SEQ ID NO: 412, SEQ ID NO: 413, SEQ ID NO: 414;
(e) HCDR1, HCDR2, and HCDR3 comprise the sequences having at least 85%, 90%, or 95% sequence identity to the amino acid sequences SEQ ID NO: 419, SEQ ID NO: 420, and SEQ ID NO: 421 and LCDR1, LCDR2, and LCDR3 comprise the sequences having at least 85%, 90%, or 95% sequence identity to the amino acid sequences SEQ ID NO: 426, SEQ ID NO: 427, SEQ ID NO: 428;
(f) HCDR1, HCDR2, and HCDR3 comprise the sequences having at least 85%, 90%, or 95% sequence identity to the amino acid sequences SEQ ID NO: 435, SEQ ID NO: 436, and SEQ ID NO: 437 and LCDR1, LCDR2, and LCDR3 comprise the sequences having at least 85%, 90%, or 95% sequence identity to the amino acid sequences SEQ ID NO: 442, SEQ ID NO: 443, SEQ ID NO: 444;
(g) HCDR1, HCDR2, and HCDR3 comprise the sequences having at least 85%, 90%, or 95% sequence identity to the amino acid sequences SEQ ID NO: 449, SEQ ID NO: 450, and SEQ ID NO: 451 and LCDR1, LCDR2, and LCDR3 comprise the sequences having at least 85%, 90%, or 95% sequence identity to the amino acid sequences SEQ ID NO: 456, SEQ ID NO: 457, SEQ ID NO: 458;
(h) HCDR1, HCDR2, and HCDR3 comprise the sequences having at least 85%, 90%, or 95% sequence identity to the amino acid sequences SEQ ID NO: 463, SEQ ID NO: 464, and SEQ ID NO: 465 and LCDR1, LCDR2, and LCDR3 comprise the sequences having at least 85%, 90%, or 95% sequence identity to the amino acid sequences SEQ ID NO: 470, SEQ ID NO: 471, SEQ ID NO: 472;
(i) HCDR1, HCDR2, and HCDR3 comprise the sequences having at least 85%, 90%, or 95% sequence identity to the amino acid sequences SEQ ID NO: 477, SEQ ID NO: 478, and SEQ ID NO: 479 and LCDR1, LCDR2, and LCDR3 comprise the sequences having at least 85%, 90%, or 95% sequence identity to the amino acid sequences SEQ ID NO: 484, SEQ ID NO: 485, SEQ ID NO: 486, or
(j) HCDR1, HCDR2, and HCDR3 comprise the sequences having at least 85%, 90%, or 95% sequence identity to the amino acid sequences SEQ ID NO: 491, SEQ ID NO: 492, and SEQ ID NO: 493 and LCDR1, LCDR2, and LCDR3 comprise the sequences having at least 85%, 90%, or 95% sequence identity to the amino acid sequences SEQ ID NO: 498, SEQ ID NO: 499, SEQ ID NO: 500.

3. The method of claim 1, wherein the anti-ICOS antibody or antigen-binding fragment thereof comprises heavy chain complimentary determining regions (HCDRs) HCDR1, HCDR2, and HCDR3, and light chain complimentary determining regions (LCDRs) LCDR1, LCDR2, and LCDR3, wherein:
(a) HCDR1 comprises the amino acid sequence SEQ ID NO: 363, HCDR2 comprises the amino acid sequence SEQ ID NO: 364, HCDR3 comprises the amino acid sequence SEQ ID NO: 365, LCDR1 comprises the amino acid sequence SEQ ID NO: 370, LCDR2 comprises the amino acid sequence SEQ ID NO: 371, and LCDR3 comprises the amino acid sequence SEQ ID NO: 372;
(b) HCDR1 comprises the amino acid sequence SEQ ID NO: 377, HCDR2 comprises the amino acid sequence SEQ ID NO: 378, HCDR3 comprises the amino acid sequence SEQ ID NO: 379, LCDR1 comprises the amino acid sequence SEQ ID NO: 384, LCDR2 comprises the amino acid sequence SEQ ID NO: 385, and LCDR3 comprises the amino acid sequence SEQ ID NO: 386;
(c) HCDR1 comprises the amino acid sequence SEQ ID NO: 391, HCDR2 comprises the amino acid sequence SEQ ID NO: 392, HCDR3 comprises the amino acid sequence SEQ ID NO: 393, LCDR1 comprises the amino acid sequence SEQ ID NO: 398, LCDR2 comprises the amino acid sequence SEQ ID NO: 399, and LCDR3 comprises the amino acid sequence SEQ ID NO: 400;
(d) HCDR1 comprises the amino acid sequence SEQ ID NO: 405, HCDR2 comprises the amino acid sequence SEQ ID NO: 406, HCDR3 comprises the amino acid sequence SEQ ID NO: 407, LCDR1 comprises the amino acid sequence SEQ ID NO: 412, LCDR2 comprises the amino acid sequence SEQ ID NO: 413, and LCDR3 comprises the amino acid sequence SEQ ID NO: 414;
(e) HCDR1 comprises the amino acid sequence SEQ ID NO: 419, HCDR2 comprises the amino acid sequence SEQ ID NO: 420, HCDR3 comprises the amino acid sequence SEQ ID NO: 421, LCDR1 comprises the amino acid sequence SEQ ID NO: 426, LCDR2 comprises the amino acid sequence SEQ ID NO: 427, and LCDR3 comprises the amino acid sequence SEQ ID NO: 428;
(f) HCDR1 comprises the amino acid sequence SEQ ID NO: 435, HCDR2 comprises the amino acid sequence SEQ ID NO: 436, HCDR3 comprises the amino acid sequence SEQ ID NO: 437, LCDR1 comprises the amino acid sequence SEQ ID NO: 442, LCDR2 comprises the amino acid sequence SEQ ID NO: 443, and LCDR3 comprises the amino acid sequence SEQ ID NO: 444;
(g) HCDR1 comprises the amino acid sequence SEQ ID NO: 449, HCDR2 comprises the amino acid sequence SEQ ID NO: 450, HCDR3 comprises the amino acid sequence SEQ ID NO: 451, LCDR1 comprises the amino acid sequence SEQ ID NO: 456, LCDR2 comprises the amino acid sequence SEQ ID NO: 457, and LCDR3 comprises the amino acid sequence SEQ ID NO: 458;
(h) HCDR1 comprises the amino acid sequence SEQ ID NO: 463, HCDR2 comprises the amino acid sequence SEQ ID NO: 464, HCDR3 comprises the amino acid sequence SEQ ID NO: 465, LCDR1 comprises the amino acid sequence SEQ ID NO: 470, LCDR2 comprises the amino acid sequence SEQ ID NO: 471, and LCDR3 comprises the amino acid sequence SEQ ID NO: 472;
(i) HCDR1 comprises the amino acid sequence SEQ ID NO: 477, HCDR2 comprises the amino acid sequence SEQ ID NO: 478, HCDR3 comprises the amino acid sequence SEQ ID NO: 479, LCDR1 comprises the amino acid sequence SEQ ID NO: 484, LCDR2 comprises the amino acid sequence SEQ ID NO: 485, and LCDR3 comprises the amino acid sequence SEQ ID NO: 486; or
(j) HCDR1 comprises the amino acid sequence SEQ ID NO: 491, HCDR2 comprises the amino acid sequence SEQ ID NO: 492, HCDR3 comprises the amino acid sequence SEQ ID NO: 493, LCDR1 comprises the amino acid sequence SEQ ID NO: 498, LCDR2 comprises the amino acid sequence SEQ ID NO: 499, and LCDR3 comprises the amino acid sequence SEQ ID NO: 500.

4. The method of claim 3, wherein:
HCDR1 comprises the amino acid sequence SEQ ID NO: 405,
HCDR2 comprises the amino acid sequence SEQ ID NO: 406,
HCDR3 comprises the amino acid sequence SEQ ID NO: 407,
LCDR1 comprises the amino acid sequence SEQ ID NO: 412,
LCDR2 comprises the amino acid sequence SEQ ID NO: 413, and
LCDR3 comprises the amino acid sequence SEQ ID NO: 414.

5. The method of claim 1, wherein the anti-ICOS antibody or antigen-binding fragment thereof comprises a heavy chain variable (VH) domain and a light chain variable (VL) domain, wherein:
(a) the VH domain comprises a sequence having at least 85%, 90%, or 95% sequence identity to the amino acid sequence SEQ ID NO: 366 and the VL domain comprises a sequence having at least 85%, 90%, or 95% sequence identity to the amino acid sequence SEQ ID NO: 373;
(b) the VH domain comprises a sequence having at least 85%, 90%, or 95% sequence identity to the amino acid sequence SEQ ID NO: 380 and the VL domain comprises a sequence having at least 85%, 90%, or 95% sequence identity to the amino acid sequence SEQ ID NO: 387;
(c) the VH domain comprises a sequence having at least 85%, 90%, or 95% sequence identity to the amino acid sequence SEQ ID NO: 394 and the VL domain comprises a sequence having at least 85%, 90%, or 95% sequence identity to the amino acid sequence SEQ ID NO: 401;
(d) the VH domain comprises a sequence having at least 85%, 90%, or 95% sequence identity to the amino acid sequence SEQ ID NO: 408 and VL domain comprises a sequence having at least 85%, 90%, or 95% sequence identity to the amino acid sequence SEQ ID NO: 415;
(e) the VH domain comprises a sequence having at least 85%, 90%, or 95% sequence identity to the amino acid sequence SEQ ID NO: 422 and the VL domain comprises a sequence having at least 85%, 90%, or 95% sequence identity to the amino acid sequence SEQ ID NO: 429;
(f) the VH domain comprises a sequence having at least 85%, 90%, or 95% sequence identity to the amino acid sequence SEQ ID NO: 438 and the VL domain comprises a sequence having at least 85%, 90%, or 95% sequence identity to the amino acid sequence SEQ ID NO: 445;
(g) the VH domain comprises a sequence having at least 85%, 90%, or 95% sequence identity to the amino acid sequence SEQ ID NO: 452 and VL domain comprises a sequence having at least 85%, 90%, or 95% sequence identity to the amino acid sequence SEQ ID NO: 459;
(h) the VH domain comprises a sequence having at least 85%, 90%, or 95% sequence identity to the amino acid sequence SEQ ID NO: 467 and the VL domain comprises a sequence having at least 85%, 90%, or 95% sequence identity to the amino acid sequence SEQ ID NO: 473;
(i) the VH domain comprises a sequence having at least 85%, 90%, or 95% sequence identity to the amino acid sequence SEQ ID NO 481; and the VL domain comprises a sequence having at least 85%, 90%, or 95% sequence identity to the amino acid sequence SEQ ID NO: 488; or
(j) the VH domain comprises a sequence having at least 85%, 90%, or 95% sequence identity to the amino acid sequence SEQ ID NO: 494 and the VL domain comprises a sequence having at least 85%, 90%, or 95% sequence identity to the amino acid sequence SEQ ID NO: 501.

6. The method of claim 5, wherein the VH domain comprises a sequence having at least 95% sequence identity to SEQ ID NO: 408 and the VL domain comprises a sequence having at least 95% sequence identity to SEQ ID NO: 415.

7. The method of claim 5, wherein:
(a) the VH domain comprises the amino acid sequence SEQ ID NO: 366 and the VL domain comprises the amino acid sequence SEQ ID NO: 373;
(b) the VH domain comprises the amino acid sequence SEQ ID NO: 380 and the VL domain comprises the amino acid sequence SEQ ID NO: 387;
(c) the VH domain comprises the amino acid sequence SEQ ID NO: 394 and the VL domain comprises the amino acid sequence SEQ ID NO: 401;
(d) the VH domain comprises the amino acid sequence SEQ ID NO: 408 and the VL domain comprises the amino acid sequence SEQ ID NO: 415;
(e) the VH domain comprises the amino acid sequence SEQ ID NO: 422 and the VL domain comprises the amino acid sequence SEQ ID NO: 429;
(f) the VH domain comprises the amino acid sequence SEQ ID NO: 438 and the VL domain comprises the amino acid sequence SEQ ID NO: 445;
(g) the VH domain comprises the amino acid sequence SEQ ID NO: 452 and the VL domain comprises the amino acid sequence SEQ ID NO: 459;
(h) the VH domain comprises the amino acid sequence SEQ ID NO: 467 and the VL domain comprises the amino acid sequence SEQ ID NO: 473;
(i) the VH domain comprises the amino acid sequence SEQ ID NO: 480 and the VL domain comprises the amino acid sequence SEQ ID NO: 487; or
(j) the VH domain comprises the amino acid sequence SEQ ID NO: 494 and the VL domain comprises the amino acid sequence SEQ ID NO: 501.

8. The method claim 5, wherein the VH domain comprises the amino acid sequence SEQ ID NO: 408 and the VL domain comprises the amino acid sequence SEQ ID NO: 415.

9-13. (canceled)

14. The method claim 1, wherein the anti-ICOS antibody is KY1044.

15-17. (canceled)

18. The method of claim 1, wherein the anti-ICOS antibody or antigen-binding fragment thereof is administered to the subject at a dose of about 0.8 mg to about 2.4 mg.

19. The method of claim 1, wherein the anti-ICOS antibody or antigen-binding fragment thereof is administered to the subject at a dose of about 2.4 mg to about 8 mg.

20-22. (canceled)

23. The method of claim 1, wherein the anti-ICOS antibody or antigen-binding fragment thereof is administered to the subject every 2-6 weeks, e.g., every 2 weeks, every 3 weeks, every 4 weeks, every 5 weeks, or every 6 weeks.

24. (canceled)

25. (canceled)

26. The method of claim 1, wherein the anti-ICOS antibody or antigen-binding fragment thereof is administered to the subject monthly.

27. (canceled)

28. The method of claim 1, further comprising administering to the subject a second therapeutic agent.

29. The method of claim 28, wherein the second therapeutic comprises an anti-PD-L1 antibody or antigen-binding fragment thereof.

30. (canceled)

31. The method of claim 29, wherein the anti-PD-L1 antibody or antigen-binding fragment thereof is administered to the subject at a dose of about 1200 mg.

32. The method of claim 29, wherein the anti-PD-L1 antibody or antigen-binding fragment thereof is administered to the subject every 2-6 weeks, e.g., every 2 weeks, every 3 weeks, every 4 weeks, every 5 weeks, or every 6 weeks.

33-36. (canceled)

37. The method of claim 29, wherein the anti-PD-L1 antibody or antigen-binding fragment thereof is co-administered to the subject with the anti-ICOS antibody or antigen-binding fragment thereof every 3 weeks.

38. The method of claim 29, wherein the anti-PD-L1 antibody or antigen-binding fragment thereof is administered to the subject in alternating doses with the anti-ICOS antibody or antigen-binding fragment thereof, e.g., wherein the anti-PD-L1 antibody or antigen-binding fragment thereof is administered every 3 weeks and the anti-ICOS antibody or antigen-binding fragment thereof is administered every 6 weeks.

39. The method of claim 1, wherein the disease or condition amenable to therapy by depleting regulatory T cells (Tregs) and/or increasing effector T cell (Teff) response comprises a tumour.

40-42. (canceled)
